(19) United States
(12) Patent Application Publication
(10) Pub. No.: US 2022/0144854 A1
(43) Pub. Date: May 12, 2022

(54) MACROCYCLIC COMPOUNDS

(71) Applicant: ZENO MANAGEMENT, INC., San Diego, CA (US)

(72) Inventors: Junhu Zhang, San Diego, CA (US); Peter Qinhua Huang, San Diego, CA (US); Kevin Duane Bunker, Escondido, CA (US); Sobhana Babu Boga, San Diego, CA (US); Sunny Abraham, San Diego, CA (US); Brant Clayton Boren, San Diego, CA (US); Wanlong Jiang, San Diego, CA (US); Sunil Paliwal, Monroe Township, NJ (US)

(21) Appl. No.: 17/310,824

(22) PCT Filed: Mar. 6, 2020
(86) PCT No.: PCT/US2020/021516

§ 371 Date: Aug. 25, 2021

Related U.S. Application Data
(60) Provisional application No. 62/815,508, filed on Mar. 8, 2019.

(51) Int. Cl. C07D 515/22

(52) U.S. Cl. CPC C07D 515/22

(57) ABSTRACT

Disclosed are macrocyclic compounds of formula (I) comprising a 2-carboxy indole ring. Such compounds, and their pharmaceutically acceptable salts, are useful as Mcl-1 (myeloid cell leukemia-1) inhibitors. The compounds may be used in treating a disease or condition, such as cancer.

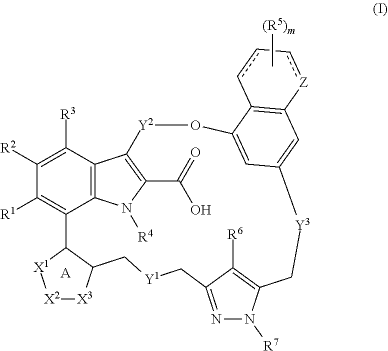

### INCORPORATION BY REFERENCE TO ANY PRIORITY APPLICATIONS

Any and all applications for which a foreign or domestic priority claim is identified, for example, in the Application Data Sheet or Request as filed with the present application, are hereby incorporated by reference under 37 CFR 1.57, and Rules 4.18 and 20.6, including U.S. Provisional Application No. 62/815,508, filed Mar. 8, 2019.

### FIELD

The present application relates to compounds that are Mcl-1 inhibitors and methods of using them to treat conditions characterized by excessive cellular proliferation, such as cancer.

### DESCRIPTION

Mcl-1 (myeloid cell leukemia-1) is a member of the Bcl-2 family of proteins. MCL-1 is widely expressed in human tissues and is primarily located in the mitochondria in cells. Upregulation of Mcl-1 occurs in different cancer types. Additionally, overexpression of Mcl-1 has been linked to drug resistance to several cancer therapies.

### SUMMARY

Some embodiments provide a compound of Formula (I), or a pharmaceutically acceptable salt thereof.

Some embodiments disclosed herein relate to a pharmaceutical composition that can include an effective amount of one or more of compounds of Formula (I), or a pharmaceutically acceptable salt thereof, and a pharmaceutically acceptable carrier, diluent, excipient or combination thereof.

Some embodiments described herein relate to a method for ameliorating and/or treating a cancer described herein that can include administering an effective amount of a compound described herein (for example, a compound of Formula (I), or a pharmaceutically acceptable salt thereof) or a pharmaceutical composition that includes an effective amount of a compound described herein (for example, a compound of Formula (I), or a pharmaceutically acceptable salt thereof) to a subject having a cancer described herein. Other embodiments described herein relate to the use of an effective amount of a compound described herein (for example, a compound of Formula (I), or a pharmaceutically acceptable salt thereof) or a pharmaceutical composition that includes an effective amount of a compound described herein (for example, a compound of Formula (I), or a pharmaceutically acceptable salt thereof) in the manufacture of a medicament for ameliorating and/or treating a cancer described herein. Still other embodiments described herein relate to an effective amount of a compound described herein (for example, a compound of Formula (I), or a pharmaceutically acceptable salt thereof) or a pharmaceutical composition that includes an effective amount of a compound described herein (for example, a compound of Formula (I), or a pharmaceutically acceptable salt thereof) for ameliorating and/or treating a cancer described herein.

Some embodiments described herein relate to a method for inhibiting replication of a malignant growth or a tumor that can include contacting the growth or the tumor with an effective amount of a compound described herein (for example, a compound of Formula (I), or a pharmaceutically acceptable salt thereof) or a pharmaceutical composition that includes an effective amount of a compound described herein (for example, a compound of Formula (I), or a pharmaceutically acceptable salt thereof), wherein the malignant growth or tumor is due to a cancer described herein. Other embodiments described herein relate to the use of an effective amount of a compound described herein (for example, a compound of Formula (I), or a pharmaceutically acceptable salt thereof) or a pharmaceutical composition that includes an effective amount of a compound described herein (for example, a compound of Formula (I), or a pharmaceutically acceptable salt thereof) in the manufacture of a medicament for inhibiting replication of a malignant growth or a tumor, wherein the malignant growth or tumor is due to a cancer described herein. Still other embodiments described herein relate to an effective amount of a compound described herein (for example, a compound of Formula (I), or a pharmaceutically acceptable salt thereof) or a pharmaceutical composition that includes an effective amount of a compound described herein (for example, a compound of Formula (I), or a pharmaceutically acceptable salt thereof) for inhibiting replication of a malignant growth or a tumor, wherein the malignant growth or tumor is due to a cancer described herein.

Some embodiments described herein relate to a method for ameliorating or treating a cancer described herein that can include contacting a malignant growth or a tumor with an effective amount of a compound described herein (for example, a compound of Formula (I), or a pharmaceutically acceptable salt thereof) or a pharmaceutical composition that includes an effective amount of a compound described herein (for example, a compound of Formula (I), or a pharmaceutically acceptable salt thereof) to a subject having a cancer described herein. Other embodiments described herein relate to the use of an effective amount of a compound described herein (for example, a compound of Formula (I), or a pharmaceutically acceptable salt thereof) or a pharmaceutical composition that includes an effective amount of a compound described herein (for example, a compound of Formula (I), or a pharmaceutically acceptable salt thereof) in the manufacture of a medicament for ameliorating or treating a cancer described herein that can include contacting a malignant growth or a tumor, wherein the malignant growth or tumor is due to a cancer described herein. Still other embodiments described herein relate to an effective amount of a compound described herein (for example, a compound of Formula (I), or a pharmaceutically acceptable salt thereof) or a pharmaceutical composition that includes an effective amount of a compound described herein (for example, a compound of Formula (I), or a pharmaceutically acceptable salt thereof) for ameliorating or treating a cancer described herein that can include contacting a malignant growth or a tumor, wherein the malignant growth or tumor is due to a cancer described herein.

Some embodiments described herein relate to a method for inhibiting the activity of Mcl-1 in a cell that can include providing an effective amount of a compound described herein (for example, a compound of Formula (I), or a pharmaceutically acceptable salt thereof) or a pharmaceutical composition that includes an effective amount of a compound described herein (for example, a compound of Formula (I), or a pharmaceutically acceptable salt thereof) to a cancer cell from a cancer described herein. Other embodiments described herein relate to the use of an effective amount of a compound described herein (for example, a compound of Formula (I), or a pharmaceutically acceptable salt thereof) or a pharmaceutical composition that includes an effective amount of a compound described herein (for example, a compound of Formula (I), or a pharmaceutically acceptable salt thereof) in the manufacture of a medicament for inhibiting the activity of Mcl-1. Still other embodiments described herein relate to an effective amount of a compound described herein (for example, a compound of Formula (I), or a pharmaceutically acceptable salt thereof) or a pharmaceutical composition that includes an effective amount of a compound described herein (for example, a compound of Formula (I), or a pharmaceutically acceptable salt thereof) for inhibiting the activity of Mcl-1.

Some embodiments described herein relate to a method for ameliorating or treating a cancer described herein that can include inhibiting the activity of Mcl-1 using an effective amount of a compound described herein (for example, a compound of Formula (I), or a pharmaceutically acceptable salt thereof) or a pharmaceutical composition that includes an effective amount of a compound described herein (for example, a compound of Formula (I), or a pharmaceutically acceptable salt thereof). Other embodiments described herein relate to the use of an effective amount of a compound described herein (for example, a compound of Formula (I), or a pharmaceutically acceptable salt thereof) or a pharmaceutical composition that includes an effective amount of a compound described herein (for example, a compound of Formula (I), or a pharmaceutically acceptable salt thereof) in the manufacture of a medicament for ameliorating or treating a cancer described herein by inhibiting the activity of Mcl-1. Still other embodiments described herein relate to an effective amount of a compound described herein (for example, a compound of Formula (I), or a pharmaceutically acceptable salt thereof) or a pharmaceutical composition that includes an effective amount of a compound described herein (for example, a compound of Formula (I), or a pharmaceutically acceptable salt thereof) for ameliorating or treating a cancer described herein by inhibiting the activity of Mcl-1.

### DETAILED DESCRIPTION

Myeloid Cell Leukemia 1 (Mcl-1) is an important anti-apoptotic member of the BCL-2 family of proteins and a master regulator of cell survival. Amplification of the MCL1 gene and/or overexpression of the Mcl-1 protein has been observed in multiple cancer types and is commonly implicated in tumor development. MCL1 is one of the most frequently amplified genes in human cancers. In many malignancies, Mcl-1 is a critical survival factor and it has been shown to mediate drug resistance to a variety of anti-cancer agents. Mcl-1 promotes cell survival by binding to pro-apoptotic proteins like Bim, Noxa, Bak, and Bax and neutralizing their death-inducing activities. Inhibition of Mcl-1 thereby releases these pro-apoptotic proteins, often leading to the induction of apoptosis in tumor cells dependent on Mcl-1 for survival. Therapeutically targeting Mcl-1 alone or in combination with other therapies, therefore, is a promising strategy to treat a multitude of malignancies and to overcome drug resistance in several human cancers.

### Definitions

Unless defined otherwise, all technical and scientific terms used herein have the same meaning as is commonly understood by one of ordinary skill in the art. All patents, applications, published applications and other publications referenced herein are incorporated by reference in their entirety unless stated otherwise. In the event that there are a plurality of definitions for a term herein, those in this section prevail unless stated otherwise.

Whenever a group is described as being “optionally substituted” that group may be unsubstituted or substituted with one or more of the indicated substituents. Likewise, when a group is described as being “unsubstituted or substituted” if substituted, the substituent(s) may be selected from one or more the indicated substituents. If no substituents are indicated, it is meant that the indicated “optionally substituted” or “substituted” group may be substituted with one or more group(s) individually and independently selected from alkyl, alkenyl, alkynyl, cycloalkyl, cycloalkenyl, aryl, heteroaryl, heterocyclyl, aryl(alkyl), cycloalkyl(alkyl), heteroaryl(alkyl), heterocyclyl(alkyl), hydroxy, alkoxy, acyl, cyano, halogen, thiocarbonyl, O-carbamyl, N-carbamyl, O-thiocarbamyl, N-thiocarbamyl, C-amido, N-amido, S-sulfonamido, N-sulfonamido, C-carboxy, O-carboxy, nitro, sulfenyl, sulfinyl, sulfonyl, haloalkyl, hydroxyalkyl, haloalkoxy, an amino, a mono-substituted amine group, a di-substituted amine group and an amine(C₁-C₆ alkyl).

As used herein, “Cₐ to C_{b}” in which “a” and “b” are integers refer to the number of carbon atoms in a group. The indicated group can contain from “a” to “b”, inclusive, carbon atoms. Thus, for example, a “C₁ to C₄ alkyl” group refers to all alkyl groups having from 1 to 4 carbons, that is, CH₃—, CH₃CH₂—, CH₃CH₂CH₂—, (CH₃)₂CH—, CH₃CH₂CH₂CH₂—, CH₃CH₂CH(CH₃)— and (CH₃)₃C—. If no “a” and “b” are designated, the broadest range described in these definitions is to be assumed.

If two “R” groups are described as being “taken together” the R groups and the atoms they are attached to can form a cycloalkyl, cycloalkenyl, aryl, heteroaryl or heterocycle. For example, without limitation, if R^{a} and R^{b} of an NR^{a}R^{b} group are indicated to be “taken together,” it means that they are covalently bonded to one another to form a ring:

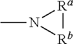

As used herein, the term “alkyl” refers to a fully saturated aliphatic hydrocarbon group. The alkyl moiety may be branched or straight chain. Examples of branched alkyl groups include, but are not limited to, iso-propyl, sec-butyl, t-butyl and the like. Examples of straight chain alkyl groups include, but are not limited to, methyl, ethyl, n-propyl, n-butyl, n-pentyl, n-hexyl, n-heptyl and the like. The alkyl group may have 1 to 30 carbon atoms (whenever it appears herein, a numerical range such as “1 to 30” refers to each integer in the given range; e.g., “1 to 30 carbon atoms” means that the alkyl group may consist of 1 carbon atom, 2 carbon atoms, 3 carbon atoms, etc., up to and including 30 carbon atoms, although the present definition also covers the occurrence of the term “alkyl” where no numerical range is designated). The alkyl group may also be a medium size alkyl having 1 to 12 carbon atoms. The alkyl group could also be a lower alkyl having 1 to 6 carbon atoms. An alkyl group may be substituted or unsubstituted.

The term “alkenyl” used herein refers to a monovalent straight or branched chain radical of from two to twenty carbon atoms containing a carbon double bond(s) including, but not limited to, 1-propenyl, 2-propenyl, 2-methyl-1-propenyl, 1-butenyl, 2-butenyl and the like. An alkenyl group may be unsubstituted or substituted.

The term “alkynyl” used herein refers to a monovalent straight or branched chain radical of from two to twenty carbon atoms containing a carbon triple bond(s) including, but not limited to, 1-propynyl, 1-butynyl, 2-butynyl and the like. An alkynyl group may be unsubstituted or substituted.

As used herein, “cycloalkyl” refers to a completely saturated (no double or triple bonds) mono- or multi-cyclic hydrocarbon ring system. When composed of two or more rings, the rings may be joined together in a fused, bridged or spiro fashion. As used herein, the term “fused” refers to two rings which have two atoms and one bond in common. As used herein, the term “bridged cycloalkyl” refers to compounds wherein the cycloalkyl contains a linkage of one or more atoms connecting non-adjacent atoms. As used herein, the term “spiro” refers to two rings which have one atom in common and the two rings are not linked by a bridge. Cycloalkyl groups can contain 3 to 30 atoms in the ring(s), 3 to 20 atoms in the ring(s), 3 to 10 atoms in the ring(s), 3 to 8 atoms in the ring(s) or 3 to 6 atoms in the ring(s). A cycloalkyl group may be unsubstituted or substituted. Examples of mono-cycloalkyl groups include, but are in no way limited to, cyclopropyl, cyclobutyl, cyclopentyl, cyclohexyl, cycloheptyl and cyclooctyl. Examples of fused cycloalkyl groups are decahydronaphthalenyl, dodecahydro-1H-phenalenyl and tetradecahydroanthracenyl; examples of bridged cycloalkyl groups are bicyclo[1.1.1]pentyl, adamantanyl and norbornanyl; and examples of spiro cycloalkyl groups include spiro[3.3]heptane and spiro[4.5]decane.

As used herein, “cycloalkenyl” refers to a mono- or multi-cyclic hydrocarbon ring system that contains one or more double bonds in at least one ring; although, if there is more than one, the double bonds cannot form a fully delocalized pi-electron system throughout all the rings (otherwise the group would be “aryl,” as defined herein). Cycloalkenyl groups can contain 3 to 10 atoms in the ring(s), 3 to 8 atoms in the ring(s) or 3 to 6 atoms in the ring(s). When composed of two or more rings, the rings may be connected together in a fused, bridged or spiro fashion. A cycloalkenyl group may be unsubstituted or substituted.

As used herein, “aryl” refers to a carbocyclic (all carbon) monocyclic or multicyclic aromatic ring system (including fused ring systems where two carbocyclic rings share a chemical bond) that has a fully delocalized pi-electron system throughout all the rings. The number of carbon atoms in an aryl group can vary. For example, the aryl group can be a C₆-C₁₄ aryl group, a C₆-C₁₀ aryl group or a C₆ aryl group. Examples of aryl groups include, but are not limited to, benzene, naphthalene and azulene. An aryl group may be substituted or unsubstituted.

As used herein, “heteroaryl” refers to a monocyclic or multicyclic aromatic ring system (a ring system with fully delocalized pi-electron system) that contain(s) one or more heteroatoms (for example, 1, 2 or 3 heteroatoms), that is, an element other than carbon, including but not limited to, nitrogen, oxygen and sulfur. The number of atoms in the ring(s) of a heteroaryl group can vary. For example, the heteroaryl group can contain 4 to 14 atoms in the ring(s), 5 to 10 atoms in the ring(s) or 5 to 6 atoms in the ring(s), such as nine carbon atoms and one heteroatom; eight carbon atoms and two heteroatoms; seven carbon atoms and three heteroatoms; eight carbon atoms and one heteroatom; seven carbon atoms and two heteroatoms; six carbon atoms and three heteroatoms; five carbon atoms and four heteroatoms; five carbon atoms and one heteroatom; four carbon atoms and two heteroatoms; three carbon atoms and three heteroatoms; four carbon atoms and one heteroatom; three carbon atoms and two heteroatoms; or two carbon atoms and three heteroatoms. Furthermore, the term “heteroaryl” includes fused ring systems where two rings, such as at least one aryl ring and at least one heteroaryl ring or at least two heteroaryl rings, share at least one chemical bond. Examples of heteroaryl rings include, but are not limited to, furan, furazan, thiophene, benzothiophene, phthalazine, pyrrole, oxazole, benzoxazole, 1,2,3-oxadiazole, 1,2,4-oxadiazole, thiazole, 1,2,3-thiadiazole, 1,2,4-thiadiazole, benzothiazole, imidazole, benzimidazole, indole, indazole, pyrazole, benzopyrazole, isoxazole, benzoisoxazole, isothiazole, triazole, benzotriazole, thiadiazole, tetrazole, pyridine, pyridazine, pyrimidine, pyrazine, purine, pteridine, quinoline, isoquinoline, quinazoline, quinoxaline, cinnoline and triazine. A heteroaryl group may be substituted or unsubstituted.

As used herein, “heterocyclyl” refers to three-, four-, five-, six-, seven-, eight-, nine-, ten-, up to 18-membered monocyclic, bicyclic and tricyclic ring system wherein carbon atoms together with from 1 to 5 heteroatoms constitute said ring system. A heterocycle may optionally contain one or more unsaturated bonds situated in such a way, however, that a fully delocalized pi-electron system does not occur throughout all the rings. The heteroatom(s) is an element other than carbon including, but not limited to, oxygen, sulfur and nitrogen. A heterocycle may further contain one or more carbonyl or thiocarbonyl functionalities, so as to make the definition include oxo-systems and thio-systems such as lactams, lactones, cyclic imides, cyclic thioimides and cyclic carbamates. When composed of two or more rings, the rings may be joined together in a fused, bridged or spiro fashion. As used herein, the term “fused” refers to two rings which have two atoms and one bond in common. As used herein, the term “bridged heterocyclyl” refers to compounds wherein the heterocyclyl contains a linkage of one or more atoms connecting non-adjacent atoms. As used herein, the term “spiro” refers to two rings which have one atom in common and the two rings are not linked by a bridge. Heterocyclyl group can contain 3 to 30 atoms in the ring(s), 3 to 20 atoms in the ring(s), 3 to 10 atoms in the ring(s), 3 to 8 atoms in the ring(s) or 3 to 6 atoms in the ring(s). For example, five carbon atoms and one heteroatom; four carbon atoms and two heteroatoms; three carbon atoms and three heteroatoms; four carbon atoms and one heteroatom; three carbon atoms and two heteroatoms; two carbon atoms and three heteroatoms; one carbon atom and four heteroatoms; three carbon atoms and one heteroatom; or two carbon atoms and one heteroatom. Additionally, any nitrogens in a heteroalicyclic may be quaternized. Heterocyclyl groups may be unsubstituted or substituted. Examples of such “heterocyclyl” groups include but are not limited to, 1,3-dioxin, 1,3-dioxane, 1,4-dioxane, 1,2-dioxolane, 1,3-dioxolane, 1,4-dioxolane, 1,3-oxathiane, 1,4-oxathiin, 1,3-oxathiolane, 1,3-dithiole, 1,3-dithiolane, 1,4-oxathiane, tetrahydro-1,4-thiazine, 2H-1,2-oxazine, maleimide, succinimide, barbituric acid, thiobarbituric acid, dioxopiperazine, hydantoin, dihydrouracil, trioxane, hexahydro-1,3,5-triazine, imidazoline, imidazolidine, isoxazoline, isoxazolidine, oxazoline, oxazolidine, oxazolidinone, thiazoline, thiazolidine, morpholine, oxirane, piperidine N-Oxide, piperidine, piperazine, pyrrolidine, azepane, pyrrolidone, pyrrolidione, 4-piperidone, pyrazoline, pyrazolidine, 2-oxopyrrolidine, tetrahydropyran, 4H-pyran, tetrahydrothiopyran, thiomorpholine, thiomorpholine sulfoxide, thiomorpholine sulfone and their benzo-fused analogs (e.g., benzimidazolidinone, tetrahydroquinoline and/or 3,4-methylenedioxyphenyl). Examples of spiro heterocyclyl groups include 2-azaspiro[3.3]heptane, 2-oxaspiro[3.3]heptane, 2-oxa-6-azaspiro[3.3]heptane, 2,6-diazaspiro[3.3]heptane, 2-oxaspiro[3.4]octane and 2-azaspiro[3.4]octane.

As used herein, “cycloalkyl(alkyl)” refer to an cycloalkyl group connected, as a substituent, via a lower alkylene group. The lower alkylene and cycloalkyl group of an cycloalkyl(alkyl) may be substituted or unsubstituted. Examples include but are not limited to cyclopropyl(alkyl), cyclobutyl(alkyl), cyclopentyl(alkyl) and cyclohexyl(alkyl).

As used herein, “aryl(alkyl)” refer to an aryl group connected, as a substituent, via a lower alkylene group. The lower alkylene and aryl group of an aryl(alkyl) may be substituted or unsubstituted. Examples include but are not limited to benzyl, 2-phenylalkyl, 3-phenylalkyl and naphthylalkyl.

As used herein, “heteroaryl(alkyl)” refer to a heteroaryl group connected, as a substituent, via a lower alkylene group. The lower alkylene and heteroaryl group of heteroaryl(alkyl) may be substituted or unsubstituted. Examples include but are not limited to 2-thienylalkyl, 3-thienylalkyl, furylalkyl, thienylalkyl, pyrrolylalkyl, pyridylalkyl, isoxazolylalkyl and imidazolylalkyl and their benzo-fused analogs.

A “heterocyclyl(alkyl)” refer to a heterocyclic group connected, as a substituent, via a lower alkylene group. The lower alkylene and heterocyclyl of a heterocyclyl(alkyl) may be substituted or unsubstituted. Examples include but are not limited tetrahydro-2H-pyran-4-yl(methyl), piperidin-4-yl(ethyl), piperidin-4-yl(propyl), tetrahydro-2H-thiopyran-4-yl(methyl) and 1,3-thiazinan-4-yl(methyl).

As used herein, “lower alkylene groups” are straight-chained —CH₂-tethering groups, forming bonds to connect molecular fragments via their terminal carbon atoms. Examples include but are not limited to methylene (—CH₂—), ethylene (—CH₂CH₂—), propylene (—CH₂CH₂CH₂—) and butylene (—CH₂CH₂CH₂CH₂—). A lower alkylene group can be substituted by replacing one or more hydrogen of the lower alkylene group and/or by substituting both hydrogens on the same carbon with a cycloalkyl group

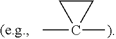

As used herein, the term “hydroxy” refers to a —OH group.

As used herein, “alkoxy” refers to the Formula —OR wherein R is an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl) is defined herein. A non-limiting list of alkoxys are methoxy, ethoxy, n-propoxy, 1-methylethoxy (iso-propoxy), n-butoxy, iso-butoxy, sec-butoxy, tert-butoxy, phenoxy and benzoxy. An alkoxy may be substituted or unsubstituted.

As used herein, “acyl” refers to a hydrogen, alkyl, alkenyl, alkynyl, aryl, heteroaryl, heterocyclyl, aryl(alkyl), heteroaryl(alkyl) and heterocyclyl(alkyl) connected, as substituents, via a carbonyl group. Examples include formyl, acetyl, propanoyl, benzoyl and acryl. An acyl may be substituted or unsubstituted.

A “cyano” group refers to a “—CN” group.

The term “halogen atom” or “halogen” as used herein, means any one of the radio-stable atoms of column 7 of the Periodic Table of the Elements, such as, fluorine, chlorine, bromine and iodine.

A “thiocarbonyl” group refers to a “—C(═S)R” group in which R can be the same as defined with respect to O-carboxy. A thiocarbonyl may be substituted or unsubstituted.

An “O-carbamyl” group refers to a “—OC(═O)N(R_{A}R_{B})” group in which R_{A} and R_{B} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). An O-carbamyl may be substituted or unsubstituted.

An “N-carbamyl” group refers to an “ROC(═O)N(R_{A})—” group in which R and R_{A} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). An N-carbamyl may be substituted or unsubstituted.

An “O-thiocarbamyl” group refers to a “—OC(═S)—N(R_{A}R_{B})” group in which R_{A} and R_{B} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). An O-thiocarbamyl may be substituted or unsubstituted.

An “N-thiocarbamyl” group refers to an “ROC(═S)N(R_{A})—” group in which R and R_{A} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). An N-thiocarbamyl may be substituted or unsubstituted.

A “C-amido” group refers to a “—C(═O)N(R_{A}R_{B})” group in which R_{A} and R_{B} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). A C-amido may be substituted or unsubstituted.

An “N-amido” group refers to a “RC(═O)N(R_{A})—” group in which R and R_{A} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). An N-amido may be substituted or unsubstituted.

An “S-sulfonamido” group refers to a “—SO₂N(R_{A}R_{B})” group in which R_{A} and R_{B} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). An S-sulfonamido may be substituted or unsubstituted.

An “N-sulfonamido” group refers to a “RSO₂N(R_{A})—” group in which R and R_{A} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). An N-sulfonamido may be substituted or unsubstituted.

An “O-carboxy” group refers to a “RC(═O)O—” group in which R can be hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl), as defined herein. An O-carboxy may be substituted or unsubstituted.

The term “C-carboxy” refer to a “—C(═O)OR” group in which R can be the same as defined with respect to O-carboxy. A C-carboxy may be substituted or unsubstituted.

A “nitro” group refers to an “—NO₂” group.

A “sulfenyl” group refers to an “—SR” group in which R can be hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). A sulfenyl may be substituted or unsubstituted.

A “sulfinyl” group refers to an “—S(═O)—R” group in which R can be the same as defined with respect to sulfenyl. A sulfinyl may be substituted or unsubstituted.

A “sulfonyl” group refers to an “SO₂R” group in which R can be the same as defined with respect to sulfenyl. A sulfonyl may be substituted or unsubstituted.

As used herein, “haloalkyl” refers to an alkyl group in which one or more of the hydrogen atoms are replaced by a halogen (e.g., mono-haloalkyl, di-haloalkyl, tri-haloalkyl and polyhaloalkyl). Such groups include but are not limited to, chloromethyl, fluoromethyl, difluoromethyl, trifluoromethyl, 1-chloro-2-fluoromethyl, 2-fluoroisobutyl and pentafluoroethyl. A haloalkyl may be substituted or unsubstituted.

As used herein, “haloalkoxy” refers to an alkoxy group in which one or more of the hydrogen atoms are replaced by a halogen (e.g., mono-haloalkoxy, di-haloalkoxy and tri-haloalkoxy). Such groups include but are not limited to, chloromethoxy, fluoromethoxy, difluoromethoxy, trifluoromethoxy, 1-chloro-2-fluoromethoxy and 2-fluoroisobutoxy. A haloalkoxy may be substituted or unsubstituted.

The term “amino” as used herein refers to a —NH₂ group.

A “mono-substituted amine” group refers to a “—NHR_{A}” group in which R_{A} can be an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl), as defined herein. The R_{A} may be substituted or unsubstituted. Examples of mono-substituted amino groups include, but are not limited to, —NH(methyl), —NH(phenyl) and the like.

A “di-substituted amine” group refers to a “—NR_{A}R_{B}” group in which R_{A} and R_{B} can be independently an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl), as defined herein. R_{A} and R_{B} can independently be substituted or unsubstituted. Examples of di-substituted amino groups include, but are not limited to, —N(methyl)₂, —N(phenyl)(methyl), —N(ethyl)(methyl) and the like.

As used herein, “amine(alkyl)” group refers to an -(alkylene)-NR′R″ radical where R′ and R″ are independently hydrogen or alkyl as defined herein. An amine(alkyl) may be substituted or unsubstituted. Examples of amine(alkyl) groups include, but are not limited to, —CH₂NH(methyl), —CH₂NH(phenyl), —CH₂CH₂NH(methyl), —CH₂CH₂NH(phenyl), —CH₂N(methyl)₂, —CH₂N(phenyl)(methyl), —NCH₂(ethyl)(methyl), —CH₂CH₂N(methyl)₂, —CH₂CH₂N(phenyl)(methyl), —NCH₂CH₂(ethyl)(methyl) and the like.

Where the number of substituents is not specified (e.g. haloalkyl), there may be one or more substituents present. For example, “haloalkyl” may include one or more of the same or different halogens. As another example, “C₁-C₃ alkoxyphenyl” may include one or more of the same or different alkoxy groups containing one, two or three atoms.

As used herein, a radical indicates species with a single, unpaired electron such that the species containing the radical can be covalently bonded to another species. Hence, in this context, a radical is not necessarily a free radical. Rather, a radical indicates a specific portion of a larger molecule. The term “radical” can be used interchangeably with the term “group.”

The term “pharmaceutically acceptable salt” refers to a salt of a compound that does not cause significant irritation to an organism to which it is administered and does not abrogate the biological activity and properties of the compound. In some embodiments, the salt is an acid addition salt of the compound. Pharmaceutical salts can be obtained by reacting a compound with inorganic acids such as hydrohalic acid (e.g., hydrochloric acid or hydrobromic acid), a sulfuric acid, a nitric acid and a phosphoric acid (such as 2,3-dihydroxypropyl dihydrogen phosphate). Pharmaceutical salts can also be obtained by reacting a compound with an organic acid such as aliphatic or aromatic carboxylic or sulfonic acids, for example formic, acetic, succinic, lactic, malic, tartaric, citric, ascorbic, nicotinic, methanesulfonic, ethanesulfonic, p-toluenesulfonic, trifluoroacetic, benzoic, salicylic, 2-oxopentanedioic or naphthalenesulfonic acid. Pharmaceutical salts can also be obtained by reacting a compound with a base to form a salt such as an ammonium salt, an alkali metal salt, such as a sodium, a potassium or a lithium salt, an alkaline earth metal salt, such as a calcium or a magnesium salt, a salt of a carbonate, a salt of a bicarbonate, a salt of organic bases such as dicyclohexylamine, N-methyl-D-glucamine, tris(hydroxymethyl)methylamine, C₁-C₇ alkylamine, cyclohexylamine, triethanolamine, ethylenediamine and salts with amino acids such as arginine and lysine. For compounds of Formula (I), those skilled in the art understand that when a salt is formed by protonation of a nitrogen-based group (for example, NH₂), the nitrogen-based group can be associated with a positive charge (for example, NH₂ can become NH₃⁺) and the positive charge can be balanced by a negatively charged counterion (such as Cl⁻).

It is understood that, in any compound described herein having one or more chiral centers, if an absolute stereochemistry is not expressly indicated, then each center may independently be of R-configuration or S-configuration or a mixture thereof. Thus, the compounds provided herein may be enantiomerically pure, enantiomerically enriched, racemic mixture, diastereomerically pure, diastereomerically enriched or a stereoisomeric mixture. In addition, it is understood that, in any compound described herein having one or more double bond(s) generating geometrical isomers that can be defined as E or Z, each double bond may independently be E or Z a mixture thereof. Likewise, it is understood that, in any compound described, all tautomeric forms are also intended to be included.

It is to be understood that where compounds disclosed herein have unfilled valencies, then the valencies are to be filled with hydrogens or isotopes thereof, e.g., hydrogen-1 (protium) and hydrogen-2 (deuterium).

It is understood that the compounds described herein can be labeled isotopically. Substitution with isotopes such as deuterium may afford certain therapeutic advantages resulting from greater metabolic stability, such as, for example, increased in vivo half-life or reduced dosage requirements. Each chemical element as represented in a compound structure may include any isotope of said element. For example, in a compound structure a hydrogen atom may be explicitly disclosed or understood to be present in the compound. At any position of the compound that a hydrogen atom may be present, the hydrogen atom can be any isotope of hydrogen, including but not limited to hydrogen-1 (protium) and hydrogen-2 (deuterium). Thus, reference herein to a compound encompasses all potential isotopic forms unless the context clearly dictates otherwise.

It is understood that the methods and combinations described herein include crystalline forms (also known as polymorphs, which include the different crystal packing arrangements of the same elemental composition of a compound), amorphous phases, salts, solvates and hydrates. In some embodiments, the compounds described herein exist in solvated forms with pharmaceutically acceptable solvents such as water, ethanol or the like. In other embodiments, the compounds described herein exist in unsolvated form. Solvates contain either stoichiometric or non-stoichiometric amounts of a solvent, and may be formed during the process of crystallization with pharmaceutically acceptable solvents such as water, ethanol or the like. Hydrates are formed when the solvent is water or alcoholates are formed when the solvent is alcohol. In addition, the compounds provided herein can exist in unsolvated as well as solvated forms. In general, the solvated forms are considered equivalent to the unsolvated forms for the purposes of the compounds and methods provided herein.

Where a range of values is provided, it is understood that the upper and lower limit, and each intervening value between the upper and lower limit of the range is encompassed within the embodiments.

Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide exemplary instances of the item in discussion, not an exhaustive or limiting list thereof; and use of terms like ‘preferably,’ ‘preferred,’ ‘desired,’ or ‘desirable,’ and words of similar meaning should not be understood as implying that certain features are critical, essential, or even important to the structure or function, but instead as merely intended to highlight alternative or additional features that may or may not be utilized in a particular embodiment. In addition, the term “comprising” is to be interpreted synonymously with the phrases “having at least” or “including at least”. When used in the context of a compound, composition or device, the term “comprising” means that the compound, composition or device includes at least the recited features or components, but may also include additional features or components.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

### Compounds

Some embodiments disclosed herein relate to a compound of Formula (I), or a pharmaceutically acceptable salt thereof, having the structure:

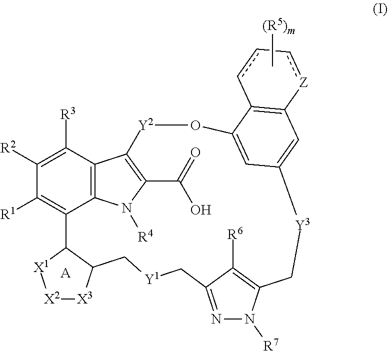

wherein: R¹, R², R³ and R⁶ can be each independently hydrogen, halogen, an unsubstituted C₁₋₄ alkyl or an unsubstituted C₁₋₄ haloalkyl; R⁴ and R⁷ can be each independently hydrogen, an optionally substituted C₁₋₄ alkyl, an optionally substituted C₃₋₆ monocyclic cycloalkyl or an unsubstituted C₁₋₄ haloalkyl; X¹, X² and X³ can be each independently NR⁸ or CR⁹; and wherein Ring A can be an aromatic ring; R⁸ and R⁹ can be each independently absent, hydrogen, halogen, cyano, an optionally substituted C₁₋₄ alkyl, an optionally substituted C₁₋₄ alkoxy, an optionally substituted C₃₋₆ monocyclic cycloalkyl, an optionally substituted C₃₋₆ bicyclic cycloalkyl, a monosubstituted amine, a disubstituted amine or a nitrogen protecting group; or the substituent attached to X¹ and the substituted attached to X² can be taken together to form Ring B fused to Ring A; and X³ can be NR⁸ or CR⁹, wherein R⁸ and R⁹ are each independently absent, hydrogen, halogen, cyano, an optionally substituted C₁₋₄ alkyl, an optionally substituted C₁₋₄ alkoxy, an optionally substituted C₃₋₆ monocyclic cycloalkyl, an optionally substituted C₃₋₆ bicyclic cycloalkyl, a monosubstituted amine, a disubstituted amine or a nitrogen protecting group; and wherein Ring A and Ring B together form an optionally substituted bicyclic heteroaryl or an optionally substituted bicyclic heterocyclyl; or the substituent attached to X² and the substituted attached to X³ can be taken together to form Ring C fused to Ring A; and X¹ can be NR⁸ or CR⁹, wherein R⁸ and R⁹ are each independently absent, hydrogen, halogen, cyano, an optionally substituted C₁₋₄ alkyl, an optionally substituted C₁₋₄ alkoxy, an optionally substituted C₃₋₆ monocyclic cycloalkyl, an optionally substituted C₃₋₆ bicyclic cycloalkyl, a monosubstituted amine, a disubstituted amine or a nitrogen protecting group; and wherein Ring A and Ring C together form an optionally substituted bicyclic heteroaryl or an optionally substituted bicyclic heterocyclyl; Y¹ can be O (oxygen), S (sulfur), SO, SO₂, CH₂, CF₂ or NR^{10A}; Y² can be an optionally substituted C₁₋₄ alkylene, and when Y² can be substituted, each substituent can be independently halogen or an unsubstituted C₁₋₄ alkyl; Y³ can be O (oxygen), S (sulfur), SO, SO₂, CH₂, CF₂ or NR^{10B}; R^{10A} and R^{10B} can be independently hydrogen or an optionally substituted C₁₋₄ alkyl; Z can be CH₂, CH or NH, wherein when Z is CH₂, then each [symbol] can be single bond; wherein when Z can be CH, then each [symbol] can be double bond; and wherein when Z can be NH, then each [symbol] can be single bond; m can be 0, 1 or 2; and each R⁵ can be independently halogen or an optionally substituted C₁₋₄ alkyl.

The phenyl ring of the indole of Formula (I) can be unsubstituted or substituted. In some embodiments, R¹, R² and R³ can each be hydrogen. When the phenyl ring of the indole ring is substituted, the phenyl ring can be mono-, di- or tri-substituted. In some embodiments, R¹ can be halogen (such as fluoro or chloro). In other embodiments, R¹ can be an unsubstituted C₁₋₄ alkyl. Examples of unsubstituted C₁₋₄ alkyls include methyl, ethyl, n-propyl, isopropyl, n-butyl, isobutyl and tert-butyl. In still other embodiments, R¹ can be an unsubstituted C₁₋₄ haloalkyl, such as CF₃ and CHF₂. In some embodiments, R² can be hydrogen. In other embodiments, R² can be halogen, including those described herein. In still other embodiments, R² can be an unsubstituted C₁₋₄ alkyl, such as those described herein. In yet still other embodiments, R² can be an unsubstituted C₁₋₄ haloalkyl. In some embodiments, R³ can be hydrogen. In other embodiments, R³ can be halogen, such as F or Cl. In still other embodiments, R³ can be an unsubstituted C₁₋₄ alkyl (for example, methyl, ethyl, n-propyl, isopropyl, n-butyl, isobutyl and tert-butyl). In yet still other embodiments, R³ can be an unsubstituted C₁₋₄ haloalkyl. In some embodiments, R¹ can be halogen, an unsubstituted C₁₋₄ alkyl or an unsubstituted C₁₋₄ haloalkyl; and R² and R³ can be each hydrogen. In other embodiments, R¹ and R³ can be independently halogen, an unsubstituted C₁₋₄ alkyl or an unsubstituted C₁₋₄ haloalkyl; and R² can be hydrogen.

The 5-membered ring of the indole can be unsubstituted or substituted. In some embodiments, R⁴ can be hydrogen. In other embodiments, R⁴ can be an unsubstituted C₁₋₄ alkyl. In still other embodiments, R⁴ can be a substituted C₁₋₄ alkyl. Suitable C₁₋₄ alkyls are described herein and include methyl, ethyl, n-propyl, isopropyl, n-butyl, isobutyl and tert-butyl. In some embodiments, R⁴ can be an unsubstituted C₃₋₆ monocyclic cycloalkyl. In other embodiments, R⁴ can be a substituted C₃₋₆ monocyclic cycloalkyl. Examples of C₃₋₆ monocyclic cycloalkyls include cyclopropyl, cyclobutyl, cyclopentyl and cyclohexyl. In still other embodiments, R⁴ can be an unsubstituted C₁₋₄ haloalkyl, such as CHF₂ and CF₃.

The pyrazole of Formula (I),

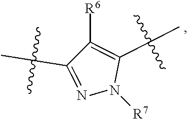

can be unsubstituted or substituted. When the pyrazole is unsubstituted, R⁶ and R⁷ can be each hydrogen. In some embodiments, the pyrazole can be substituted, wherein at least one of R⁶ and R⁷ is a non-hydrogen substituent. In some embodiments, R⁶ can be hydrogen. In other embodiments, R⁶ can be halogen. In still other embodiments, R⁶ can be an unsubstituted C₁₋₄ alkyl. In yet still other embodiments, R⁶ can be an unsubstituted C₁₋₄ haloalkyl. In some embodiments, R⁷ can be hydrogen. In other embodiments, R⁷ can be an unsubstituted C₁₋₄ alkyl. In still other embodiments, R⁷ can be a substituted C₁₋₄ alkyl. In yet still other embodiments, R⁷ can be an unsubstituted C₃₋₆ monocyclic cycloalkyl. In some embodiments, R⁷ can be a substituted C₃₋₆ monocyclic cycloalkyl. In other embodiments, R⁷ can be an unsubstituted C₁₋₄ haloalkyl. Examples of C₁₋₄ alkyl, C₃₋₆ monocyclic cycloalkyl and C₁₋₄ haloalkyls are described herein. Several examples of

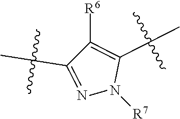

include the following:

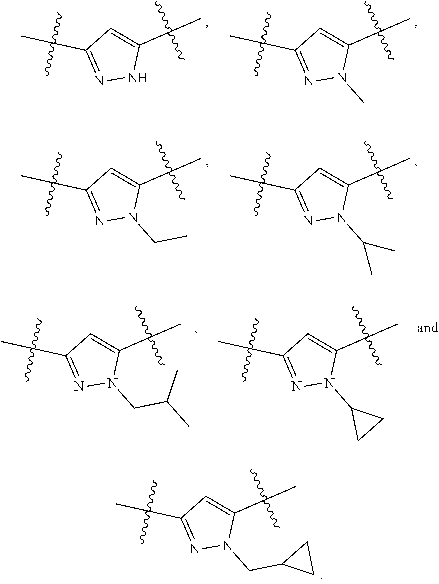

As described herein, Ring A can be a monocyclic aromatic ring, or when taken together with a second ring (such as Ring B or Ring C), Ring A together with the second ring can be an optionally substituted heteroaryl or an optionally substituted heterocyclyl. In some embodiments, X¹, X² and X³ can be each independently NR⁸ or CR⁹; and Ring A can be an aromatic ring, wherein R⁸ and R⁹ can be each independently absent, hydrogen, halogen, cyano, an optionally substituted C₁₋₄ alkyl, an optionally substituted C₁₋₄ alkoxy, an optionally substituted C₃₋₆ monocyclic cycloalkyl, an optionally substituted C₃₋₆ bicyclic cycloalkyl, a monosubstituted amine, a disubstituted amine or a nitrogen protecting group. In some embodiments, at least one of X¹, X² and X³ is NR⁸. In some embodiments, X¹ can be CR⁹; and X² and X³ can be each NR⁸. In other embodiments, X¹ and X³ can be each CR⁹; and X² can be NR⁸. In still other embodiments, X¹ and X³ can be each NR⁸; and X² can be CR⁹. In yet still other embodiments, X¹ and X² can be each NR⁸; and X³ can be CR⁹.

As provided herein, R⁸ and R⁹ can be a non-hydrogen group. For example, R⁸ and R⁹ can be independently halogen, cyano, an unsubstituted C₁₋₄ alkyl, an unsubstituted C₁₋₄ alkoxy, an unsubstituted C₃₋₆ monocyclic cycloalkyl, an unsubstituted C₃₋₆ bicyclic cycloalkyl, a monosubstituted amine or a disubstituted amine. In some embodiments, each R⁹ can be methyl; and R⁸ can be an unsubstituted C₁₋₄ alkyl, an unsubstituted C₃₋₆ monocyclic cycloalkyl or an unsubstituted C₃₋₆ bicyclic cycloalkyl (such as bicyclo[1.1.1]pentyl). In some embodiments, one R⁹ can be methyl; one R⁹ can be cyano; and R⁸ can be an unsubstituted C₁₋₄ alkyl, an unsubstituted C₃₋₆ monocyclic cycloalkyl or an unsubstituted C₃₋₆ bicyclic cycloalkyl (such as bicyclo[1.1.1]pentyl).

Various examples of Ring A being a monocyclic aromatic ring include the following:

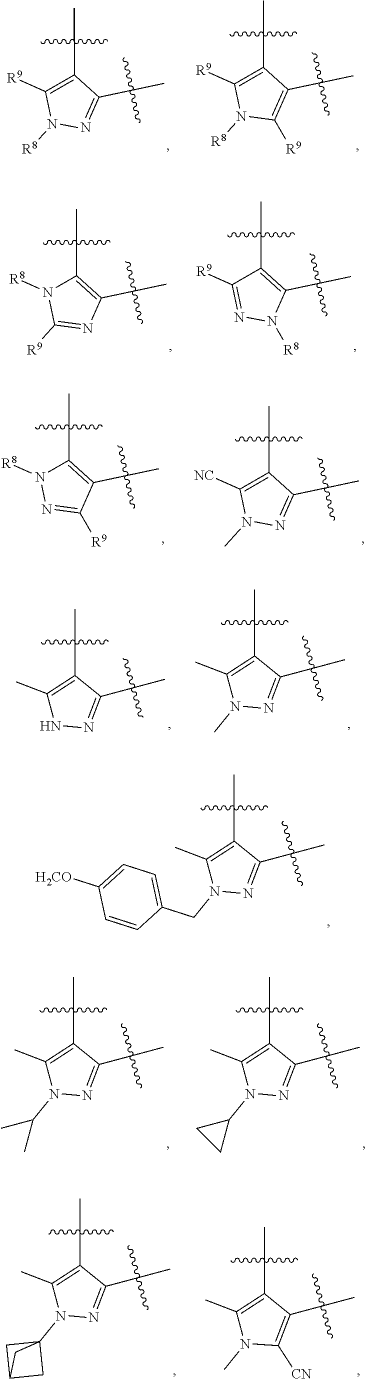

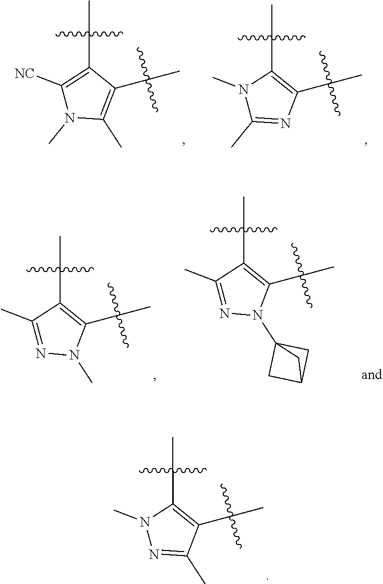

In other embodiments, X¹ and X² can be each independently NR⁸ or CR⁹; the substituent attached to X¹ and the substituted attached to X² can be taken together to form Ring B fused to Ring A; X³ can be NR⁸ or CR⁹; Ring A and Ring B can form an optionally substituted heteroaryl or an optionally substituted heterocyclyl; and R⁸ and R⁹ can be each independently absent, hydrogen, halogen, cyano, an optionally substituted C₁₋₄ alkyl, an optionally substituted C₁₋₄ alkoxy, an optionally substituted C₃₋₆ monocyclic cycloalkyl, an optionally substituted C₃₋₆ bicyclic cycloalkyl, a monosubstituted amine, a disubstituted amine or a nitrogen protecting group. In some embodiments, X¹ and X² can be each independently NR⁸ or CR⁹; X³ can be NR⁸; and Ring A and Ring B can form an optionally substituted heteroaryl. In other embodiments, X¹ and X² can be each independently NR⁸ or CR⁹; X³ can be NR⁸; and Ring A and Ring B can form an optionally substituted heterocyclyl. In still other embodiments, X¹ and X² can be each independently NR⁸ or CR⁹; X³ can be CR⁹; and Ring A and Ring B can form an optionally substituted heteroaryl. In yet still other embodiments, X¹ and X² can be each independently NR⁸ or CR⁹; X³ can be CR⁹; and Ring A and Ring B can form an optionally substituted heterocyclyl. In some embodiments, X¹ can be CR⁹; X² can be NR⁸; X³ can be NR⁸; and Ring A and Ring B can form an optionally substituted heteroaryl. In other embodiments, X¹ can be CR⁹; X² can be NR⁸; X³ can be NR⁸; and Ring A and Ring B can form an optionally substituted heterocyclyl. Ring B can be a 5- to 6-membered ring. Examples of the rings of this paragraph are:

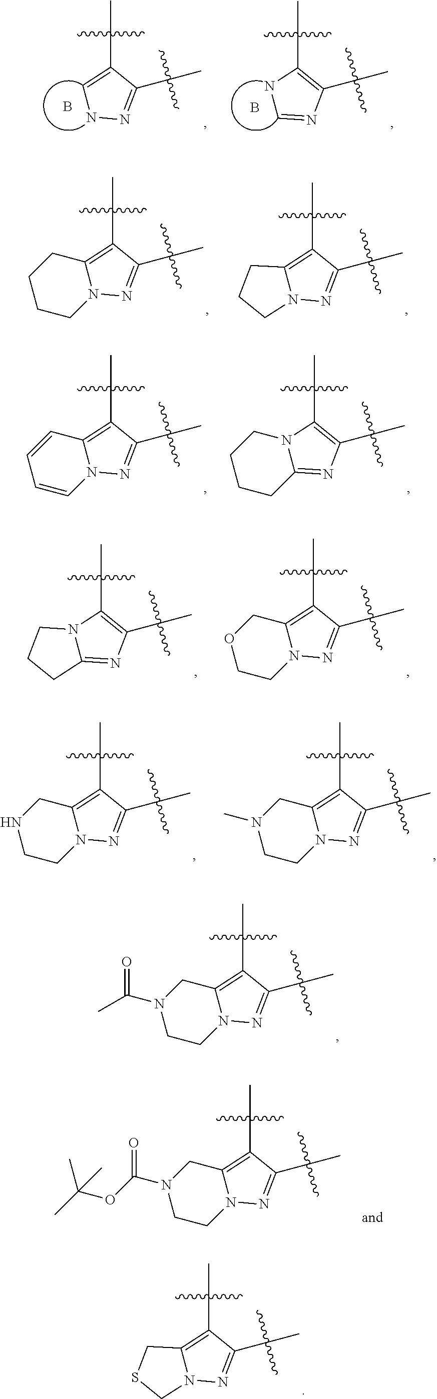

The aforementioned rings can be further substituted with substituents such as those described for “optionally substituted.”

In other embodiments, X² and X³ can be each independently NR⁸ or CR⁹; the substituent attached to X² and the substituted attached to X³ can be taken together to form Ring C fused to Ring A; X¹ can be NR⁸ or CR⁹; Ring A and Ring C can form an optionally substituted heteroaryl or an optionally substituted heterocyclyl; and R⁸ and R⁹ can be each independently absent, hydrogen, halogen, cyano, an optionally substituted C₁₋₄ alkyl, an optionally substituted C₁₋₄ alkoxy, an optionally substituted C₃₋₆ monocyclic cycloalkyl, an optionally substituted C₃₋₆ bicyclic cycloalkyl, a monosubstituted amine, a disubstituted amine or a nitrogen protecting group. In some embodiments, X² and X³ can be each independently NR⁸ or CR⁹; X¹ can be NR⁸; and Ring A and Ring C can form an optionally substituted heteroaryl. In other embodiments, X² and X³ can be each independently NR⁸ or CR⁹; X¹ can be NR⁸; and Ring A and Ring C can form an optionally substituted heterocyclyl. In still other embodiments, X² and X³ can be each independently NR⁸ or CR⁹; X¹ can be CR⁹; and Ring A and Ring C can form an optionally substituted heteroaryl. In yet still other embodiments, X² and X³ can be each independently NR⁸ or CR⁹; X¹ can be CR⁹; and Ring A and Ring C can form an optionally substituted heterocyclyl. In some embodiments, X¹ can be CR⁹; X² can be NR⁸; X³ can be NR⁸; and Ring A and Ring C can form an optionally substituted heteroaryl. In other embodiments, X¹ can be CR⁹; X² can be NR⁸; X³ can be NR⁸; and Ring A and Ring C can form an optionally substituted heterocyclyl. Examples of the rings of this paragraph are:

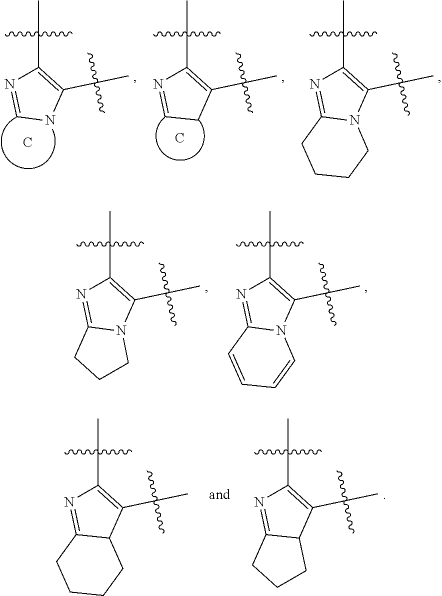

These examples of rings can be further substituted with substituents such as those described for “optionally substituted.”

In some embodiments, Z can be CH₂; and each [symbol] can be a single bond. In other embodiments, Z can be CH; and each [symbol] can be a double bond. In still other embodiments, Z can be NH; and each [symbol] can be a single bond. Examples of

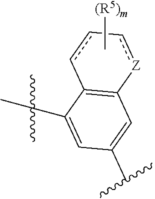

can be

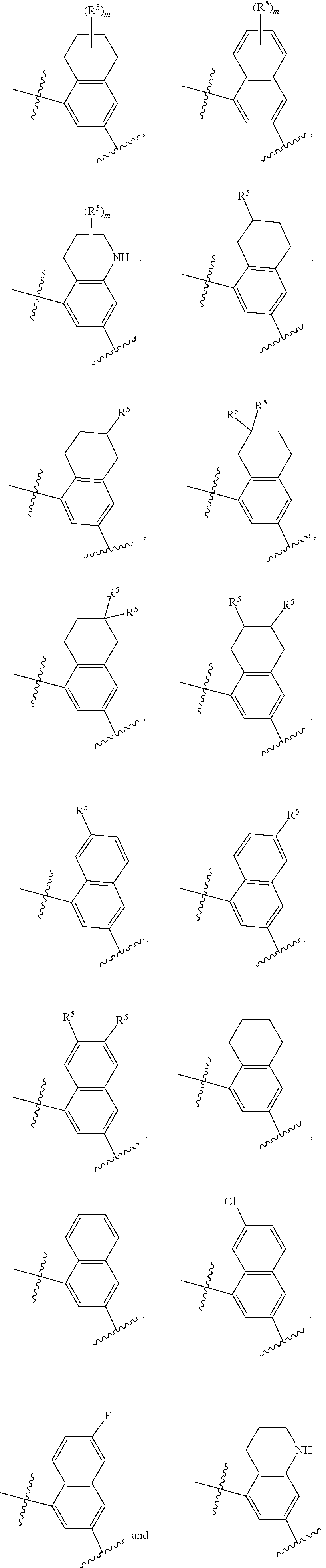

These examples of rings can be further substituted with substituents such as those described for “optionally substituted.”

In some embodiments, m can be 0, such that upper ring is unsubstituted. In other embodiments, m can be 1, wherein R⁵ can be halogen or an optionally substituted C₁₋₄ alkyl. In still other embodiments, m can be 2, wherein each R⁵ can be independently halogen or an optionally substituted C₁₋₄ alkyl. Suitable halogens (including fluoro and chloro) and an optionally substituted C₁₋₄ alkyls (optionally substituted versions of methyl, ethyl, n-propyl, isopropyl, n-butyl, isobutyl and tert-butyl). In some embodiments, each R⁵ can be independently an unsubstituted C₁₋₄ alkyl. In other embodiments, each R⁵ can be independently a substituted C₁₋₄ alkyl.

In some embodiments, Y¹ can be O (oxygen). In other embodiments, Y¹ can be S (sulfur). In still other embodiments, Y¹ can be SO. In yet still other embodiments, Y¹ can be SO₂. In some embodiments, Y¹ can be CH₂. In other embodiments, Y¹ can be CF₂. In other embodiments, Y¹ can be NR^{10A}, wherein R^{10A} can be hydrogen. In still other embodiments, Y¹ can be NR^{10A}, wherein R^{10A} can be an unsubstituted C₁₋₄ alkyl. In yet still other embodiments, Y¹ can be NR^{10A}, wherein R^{10A} can be a substituted C₁₋₄ alkyl. Examples of optionally substituted C₁₋₄ alkyls include substituted versions of methyl, ethyl, n-propyl, isopropyl, n-butyl, isobutyl or tert-butyl.

In some embodiments, Y² can be an unsubstituted C₁₋₄ alkylene. In other embodiments, Y² can be a substituted C₁₋₄ alkylene, wherein when Y² can be substituted, each substituent can be independently halogen or an unsubstituted C₁₋₄ alkyl. Exemplary optionally substituted C₁₋₄ alkylenes for Y² include: —CH₂—, —CH₂CH₂—, —CH₂CH₂CH₂—, —CH₂CH₂CH₂CH₂—, —CH(CH₃)CH₂CH₂—, —CHFCH₂CH₂— and —CH₂CF₂CH₂—.

In some embodiments, Y³ can be O (oxygen). In other embodiments, Y³ can be S (sulfur). In still other embodiments, Y³ can be SO. In yet still other embodiments, Y³ can be SO₂. In some embodiments, Y³ can be CH₂. In other embodiments, Y³ can be CF₂. In other embodiments, Y¹ can be NH. In still other embodiments, Y³ can be NR^{10B}, wherein R^{10B} can be an unsubstituted C₁₋₄ alkyl. In yet still other embodiments, Y³ can be NR^{10B}, wherein R^{10B} can be a substituted C₁₋₄ alkyl. Suitable optionally substituted C₁₋₄ alkyls include substituted versions of methyl, ethyl, n-propyl, isopropyl, n-butyl, isobutyl or tert-butyl.

In some embodiments, when Y¹, Y² and Y³ are: (1) Y¹ and Y³ are each S and Y² is —(CH₂)₃—; (2) Y¹ is S, Y² is —(CH₂)₃— and Y³ is —(CH₂)—; (3) Y¹ is NR^{10A}, Y² is —(CH₂)₃— and Y³ is S; or (4) Y¹ is NR^{10A}, Y² is —(CH₂)₃— and Y³ is —(CH₂)—; R¹ is chloro; R², R³ and R⁶ are each hydrogen; R⁴ and R⁷ are each methyl; Z is CH and each [symbol] is a double bond; or Z is NH and each [symbol] is a single bond; and m is 0; then X¹, X² and X³ are not (1) X¹ is CR⁸, wherein R⁸ is an optionally substituted C₁₋₄ alkyl, X² is N and X³ is N(CH₃); and (2) X¹ is CR⁸, wherein R⁸ is an optionally substituted C₁₋₄ alkyl, X² is N(CH₃) and X³ is N.

In some embodiments, when Y¹ and Y³ are each S and Y² is —(CH₂)₃—; R¹ is chloro; R², R³ and R⁶ are each hydrogen; R⁴ and R⁷ are each methyl; Z is CH and each [symbol] is a double bond; and m is 0; then X¹, X² and X³ are not the following: X¹ is CR⁸, wherein R⁸ is an optionally substituted C₁₋₄ alkyl, X² is N and X³ is N(CH₃).

In other embodiments, when Y¹ and Y³ are each S and Y² is —(CH₂)₃—; R¹ is chloro; R², R³ and R⁶ are each hydrogen; R⁴ and R⁷ are each methyl; Z is NH and each [symbol] is a single bond; and m is 0; then X¹, X² and X³ are not the following: X¹ is CR⁸, wherein R⁸ is an optionally substituted C₁₋₄ alkyl, X² is N and X³ is N(CH₃).

In still other embodiments, when Y¹ and Y³ are each S and Y² is —(CH₂)₃—; R¹ is chloro; R², R³ and R⁶ are each hydrogen; R⁴ and R⁷ are each methyl; Z is CH and each [symbol] is a double bond; and m is 0; then X¹, X² and X³ are not the following: X¹ is CR⁸, wherein R⁸ is an optionally substituted C₁₋₄ alkyl, X² is N(CH₃) and X³ is N.

In yet still other embodiments, when Y¹ and Y³ are each S and Y² is (CH₂)₃—; R¹ is chloro; R², R³ and R⁶ are each hydrogen; R⁴ and R⁷ are each methyl; Z is NH and each [symbol] is a single bond; and m is 0; then X¹, X² and X³ are not the following: X¹ is CR⁸, wherein R⁸ is an optionally substituted C₁₋₄ alkyl, X² is N(CH₃) and X³ is N.

In some embodiments, when Y¹ is S, Y² is —(CH₂)₃— and Y³ is —(CH₂)—; R¹ is chloro; R², R³ and R⁶ are each hydrogen; R⁴ and R⁷ are each methyl; Z is CH and each [symbol] is a double bond; and m is 0; then X¹, X² and X³ are not the following: X¹ is CR⁸, wherein R⁸ is an optionally substituted C₁₋₄ alkyl, X² is N and X³ is N(CH₃). In other embodiments, when Y¹ is S, Y² is —(CH₂)₃— and Y³ is —(CH₂)—; R¹ is chloro; R², R³ and R⁶ are each hydrogen; R⁴ and R⁷ are each methyl; Z is CH and each [symbol] is a double bond; and m is 0; then X¹, X² and X³ are not the following: X¹ is C—CH₃, X² is N and X³ is N(CH₃). In still other embodiments, when Y¹ is S, Y² is —(CH₂)₃— and Y³ is —(CH₂)—; R¹ is chloro; R², R³ and R⁶ are each hydrogen; R⁴ and R⁷ are each methyl; Z is NH and each [symbol] is a single bond; and m is 0; then X¹, X² and X³ are not the following: X¹ is CR⁸, wherein R⁸ is an optionally substituted C₁₋₄ alkyl, X² is N and X³ is N(CH₃). In yet still other embodiments, when Y¹ is S, Y² is —(CH₂)₃— and Y³ is —(CH₂)—; R¹ is chloro; R², R³ and R⁶ are each hydrogen; R⁴ and R⁷ are each methyl; Z is NH and each [symbol] is a single bond; and m is 0; then X¹, X² and X³ are not the following: X¹ is CR⁸, wherein R⁸ is an unsubstituted C₁₋₄ alkyl (for example, —CH₃), X² is N and X³ is N(CH₃).

In some embodiments, when Y¹ is S, Y² is —(CH₂)₃— and Y³ is —(CH₂)—; R¹ is chloro; R², R³ and R⁶ are each hydrogen; R⁴ and R⁷ are each methyl; Z is CH and each [symbol] is a double bond; and m is 0; then X¹, X² and X³ are not the following: X¹ is CR⁸, wherein R⁸ is an optionally substituted C₁₋₄ alkyl, X² is N(CH₃) and X³ is N. In other embodiments, when Y¹ is S, Y² is —(CH₂)₃— and Y³ is —(CH₂)—; R¹ is chloro; R², R³ and R⁶ are each hydrogen; R⁴ and R⁷ are each methyl; Z is CH and each [symbol] is a double bond; and m is 0; then X¹, X² and X³ are not the following: X¹ is CR⁸, wherein R⁸ is an unsubstituted C₁₋₄ alkyl (such as methyl), X² is N(CH₃) and X³ is N. In still other embodiments, when Y¹ is S, Y² is —(CH₂)₃— and Y³ is —(CH₂)—; R¹ is chloro; R², R³ and R⁶ are each hydrogen; R⁴ and R⁷ are each methyl; Z is NH and each [symbol] is a single bond; and m is 0; then X¹, X² and X³ are not the following: X¹ is CR⁸, wherein R⁸ is an optionally substituted C₁₋₄ alkyl, X² is N(CH₃) and X³ is N. In yet still other embodiments, when Y¹ is S, Y² is —(CH₂)₃— and Y³ is —(CH₂)—; R¹ is chloro; R², R³ and R⁶ are each hydrogen; R⁴ and R⁷ are each methyl; Z is NH and each [symbol] is a single bond; and m is 0; then X¹, X² and X³ are not the following: X¹ is CR⁸, wherein R⁸ is an unsubstituted C₁₋₄ alkyl (such as methyl), X² is N(CH₃) and X³ is N.

In some embodiments, when Y¹ is NR^{10A}, Y² is —(CH₂)₃— and Y³ is S; R¹ is chloro; R², R³ and R⁶ are each hydrogen; R⁴ and R⁷ are each methyl; Z is CH and each [symbol] is a double bond; and m is 0; then X¹, X² and X³ are not the following: X¹ is CR⁸, wherein R⁸ is an optionally substituted C₁₋₄ alkyl, X² is N and X³ is N(CH₃). In other embodiments, when Y¹ is NH, NCH₃ or NCH₂CH₃, Y² is —(CH₂)₃— and Y³ is S; R¹ is chloro; R², R³ and R⁶ are each hydrogen; R⁴ and R⁷ are each methyl; Z is CH and each [symbol] is a double bond; and m is 0; then X¹, X² and X³ are not the following: X¹ is CR⁸, wherein R⁸ is an unsubstituted C₁₋₄ alkyl (for example, —CH₃), X² is N and X³ is N(CH₃). In still other embodiments, when Y¹ is NR^{10A}, Y² is —(CH₂)₃— and Y³ is S; R¹ is chloro; R², R³ and R⁶ are each hydrogen; R⁴ and R⁷ are each methyl; Z is NH and each [symbol] is a single bond; and m is 0; then X¹, X² and X³ are not the following: X¹ is CR⁸, wherein R⁸ is an optionally substituted C₁₋₄ alkyl, X² is N and X³ is N(CH₃). In yet still other embodiments, when Y¹ is NH, NCH₃ or NCH₂CH₃, Y² is —(CH₂)₃— and Y³ is S; R¹ is chloro; R², R³ and R⁶ are each hydrogen; R⁴ and R⁷ are each methyl; Z is NH and each [symbol] is a single bond; and m is 0; then X¹, X² and X³ are not the following: X¹ is CR⁸, wherein R⁸ is an unsubstituted C₁₋₄ alkyl (such as methyl), X² is N and X³ is N(CH₃).

In some embodiments, when Y¹ is NR^{1A}, Y² is —(CH₂)₃— and Y³ is S; R¹ is chloro; R², R³ and R⁶ are each hydrogen; R⁴ and R⁷ are each methyl; Z is CH and each [symbol] is a double bond; and m is 0; then X¹, X² and X³ are not the following: X¹ is CR⁸, wherein R⁸ is an optionally substituted C₁₋₄ alkyl, X² is N(CH₃) and X³ is N. In other embodiments, when Y¹ is NH, NCH₃ or NCH₂CH₃, Y² is —(CH₂)₃— and Y³ is S; R¹ is chloro; R², R³ and R⁶ are each hydrogen; R⁴ and R⁷ are each methyl; Z is CH and each [symbol] is a double bond; and m is 0; then X¹, X² and X³ are not the following: X¹ is CR⁸, wherein R⁸ is an unsubstituted C₁₋₄ alkyl (including methyl), X² is N(CH₃) and X³ is N. In still other embodiments, when Y¹ is NR^{10A}, Y² is —(CH₂)₃— and Y³ is S; R¹ is chloro; R², R³ and R⁶ are each hydrogen; R⁴ and R⁷ are each methyl; Z is NH and each [symbol] is a single bond; and m is 0; then X¹, X² and X³ are not the following: X¹ is CR⁸, wherein R⁸ is an optionally substituted C₁₋₄ alkyl, X² is N(CH₃) and X³ is N. In yet still other embodiments, when Y¹ is NH, NCH₃ or NCH₂CH₃, Y² is —(CH₂)₃— and Y³ is S; R¹ is chloro; R², R³ and R⁶ are each hydrogen; R⁴ and R⁷ are each methyl; Z is NH and each [symbol] is a single bond; and m is 0; then X¹, X² and X³ are not the following: X¹ is CR⁸, wherein R⁸ is an unsubstituted C₁₋₄ alkyl (such as —CH₃), X² is N(CH₃) and X³ is N.

In some embodiments, when Y¹ is NR^{10A}, Y² is —(CH₂)₃— and Y³ is —(CH₂)—; R¹ is chloro; R², R³ and R⁶ are each hydrogen; R⁴ and R⁷ are each methyl; Z is CH and each [symbol] is a double bond; and m is 0; then X¹, X² and X³ are not the following: X¹ is CR⁸, wherein R⁸ is an optionally substituted C₁₋₄ alkyl (such as an unsubstituted C₁₋₄ alkyl), X² is N and X³ is N(CH₃). In other embodiments, when Y¹ is NH, NCH₃ or NCH₂CH₃, Y² is —(CH₂)₃— and Y³ is —(CH₂)—; R¹ is chloro; R², R³ and R⁶ are each hydrogen; R⁴ and R⁷ are each methyl; Z is CH and each [symbol] is a double bond; and m is 0; then X¹, X² and X³ are not the following: X¹ is C—CH₃, X² is N and X³ is N(CH₃). In some embodiments, when Y¹ is NR^{10A}, Y² is —(CH₂)₃— and Y³ is —(CH₂)—; R¹ is chloro; R², R³ and R⁶ are each hydrogen; R⁴ and R⁷ are each methyl; Z is NH and each [symbol] is a single bond; and m is 0; then X¹, X² and X³ are not the following: X¹ is CR⁸, wherein R⁸ is an optionally substituted C₁₋₄ alkyl (for example, an unsubstituted C₁₋₄ alkyl), X² is N and X³ is N(CH₃). In other embodiments, when Y¹ is NH, NCH₃ or NCH₂CH₃, Y² is —(CH₂)₃— and Y³ is —(CH₂)—; R¹ is chloro; R², R³ and R⁶ are each hydrogen; R⁴ and R⁷ are each methyl; Z is NH and each [symbol] is a single bond; and m is 0; then X¹, X² and X³ are not the following: X¹ is C—CH₃, X² is N and X³ is N(CH₃).

In some embodiments, when Y¹ is NR^{10A}, Y² is —(CH₂)₃— and Y³ is —(CH₂)—; R¹ is chloro; R², R³ and R⁶ are each hydrogen; R⁴ and R⁷ are each methyl; Z is CH and each [symbol] is a double bond; and m is 0; then X¹, X² and X³ are not the following: X¹ is CR⁸, wherein R⁸ is an optionally substituted C₁₋₄ alkyl (such as an unsubstituted C₁₋₄ alkyl), X² is N(CH₃) and X³ is N. In other embodiments, when Y¹ is NH, NCH₃ or NCH₂CH₃, Y² is —(CH₂)₃— and Y³ is —(CH₂)—; R¹ is chloro; R², R³ and R⁶ are each hydrogen; R⁴ and R⁷ are each methyl; Z is CH and each [symbol] is a double bond; and m is 0; then X¹, X² and X³ are not the following: X¹ is C—CH₃, wherein R⁸ is an optionally substituted C₁₋₄ alkyl, X² is N(CH₃) and X³ is N. In some embodiments, when Y¹ is NR^{10A}, Y² is —(CH₂)₃— and Y³ is —(CH₂)—; R¹ is chloro; R², R³ and R⁶ are each hydrogen; R⁴ and R⁷ are each methyl; Z is NH and each [symbol] is a single bond; and m is 0; then X¹, X² and X³ are not the following: X¹ is CR⁸, wherein R⁸ is an optionally substituted C₁₋₄ alkyl (for example, an unsubstituted C₁₋₄ alkyl), X² is N(CH₃) and X³ is N. In other embodiments, when Y¹ is NH, NCH₃ or NCH₂CH₃, Y² is —(CH₂)₃— and Y³ is —(CH₂)—; R¹ is chloro; R², R³ and R⁶ are each hydrogen; R⁴ and R⁷ are each methyl; Z is NH and each [symbol] is a single bond; and m is 0; then X¹, X² and X³ are not the following: X¹ is C—CH₃, wherein R⁸ is an optionally substituted C₁₋₄ alkyl, X² is N(CH₃) and X³ is N.

In some embodiments, the indole of a compound of Formula (I), or a pharmaceutically acceptable salt thereof, cannot be

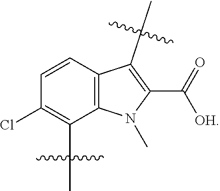 [SMILES: *c1c(C(=O)O)n(C)c2c(*)c(Cl)ccc12]

In some embodiments, Y² cannot be —(CH₂)₃—. In some embodiments, when Y¹ and Y³ are each S, then Y² cannot be —(CH₂)₃—. In other embodiments, when Y¹ is S and Y³ is —(CH₂)—, then Y² cannot be —(CH₂)₃—. In still other embodiments, when Y¹ is Y¹ is NR^{10A} (such as NH, NCH₃ and/or NCH₂CH₃) and Y³ is —(CH₂)—, then Y² cannot be —(CH₂)₃—. In some embodiments, m cannot be 0. In some embodiments, when X¹ is CR⁸, wherein R⁸ is an optionally substituted C₁₋₄ alkyl, X² is N, then X³ cannot be N(CH₃). In some embodiments, when X¹ is CR⁸, wherein R⁸ is an unsubstituted C₁₋₄ alkyl (for example, methyl), X² is N, then X³ cannot be N(CH₃). In some embodiments, when X¹ is CR⁸, wherein R⁸ is an optionally substituted C₁₋₄ alkyl, X² is N(CH₃), then X³ cannot be N (nitrogen). In some embodiments, when X¹ is CR⁸, wherein R⁸ is an unsubstituted C₁₋₄ alkyl (such as —CH₃), X² is N(CH₃), then X³ cannot be N (nitrogen). In some embodiments, the pyrazole of a compound of Formula (I), or a pharmaceutically acceptable salt thereof, cannot be

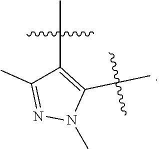 [SMILES: *c1c(C)nn(C)c1*]

In some embodiments, the pyrazole of a compound of Formula (I), or a pharmaceutically acceptable salt thereof, cannot be

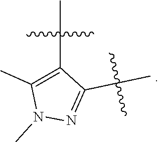 [SMILES: *c1nn(C)c(C)c1*]

In some embodiments,

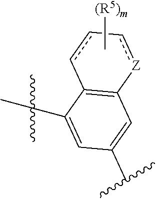

cannot be

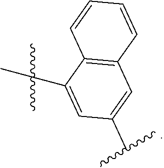 [SMILES: *c1cc(*)c2ccccc2c1]

In other embodiments,

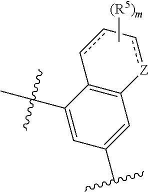

cannot be

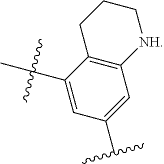 [SMILES: *c1cc(*)c2c(c1)NCCC2]

In some embodiments,

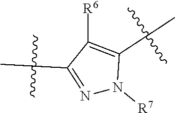

cannot be

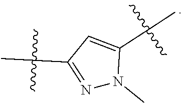 [SMILES: *c1cc(*)n(C)n1]

In some embodiments, a compound of Formula (I), or a pharmaceutically salt thereof, cannot be 17-chloro-5,13,14,22-tetramethyl-28-oxa-2,9-dithia-5,6,12,13,22-pentaazaheptocyclo[27.7.1.^{14,7}.0^{11,15}.0^{16,21}.0^{20,24}.0^{30,35}]octatriaconta-1(37),4(38),6,11,14,16,18,20,23,29,31,33,35-tridecaene-23-carboxylic acid (including (Rₐ)-17-chloro-5,13,14,22-tetramethyl-28-oxa-2,9-dithia-5,6,12,13,22-pentaazaheptocyclo[27.7.1.^{14,7}.0^{11,15}.0^{16,21}.0^{20,24}.0^{30,35}]octatriaconta-1(37),4(38),6,11,14,16,18,20,23,29,31,33,35-tridecaene-23-carboxylic acid and (Sₐ)-17-chloro-5,13,14,22-tetramethyl-28-oxa-2,9-dithia-5,6,12,13,22-pentaazaheptocyclo[27.7.1.^{14,7}.0^{11,15}.0^{16,21}.0^{20,24}.0^{30,35}]octatriaconta-1(37),4(38),6,11,14,16,18,20,23,29,31,33,35-tridecaene-23-carboxylic acid), or a pharmaceutically acceptable salt thereof (such as a sodium salt and a meglumine salt). 17-chloro-5,13,14,22-tetramethyl-28-oxa-2,9-dithia-5,6,12,13,22-pentaazaheptocyclo[27.7.1.^{14,7}.0^{11,15}.0^{16,21}.0^{20,24}.0^{30,35}]octatriaconta-1(37),4(38),6,11,14,16,18,20,23,29,31,33,35-tridecaene-23-carboxylic acid has the following structure

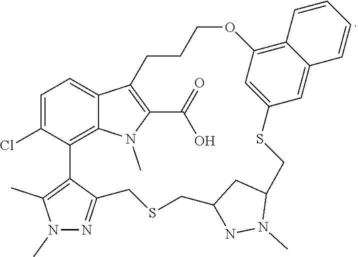 [SMILES: Cc1c2c(nn1C)CSCC1CC(CSc3cc(c4ccccc4c3)OCCCc3c(C(=O)O)n(C)c4c-2c(Cl)ccc34)N(C)N1]

In some embodiments, a compound of Formula (I), or a pharmaceutically acceptable salt thereof, cannot a compound disclosed in WO 2018/178226 that would be encompassed by a compound of Formula (I), or a pharmaceutically acceptable salt thereof. In some embodiments, a compound of Formula (I), or a pharmaceutically acceptable salt thereof, cannot a compound disclosed in WO 2017/181625 that would be encompassed by a compound of Formula (I), or a pharmaceutically acceptable salt thereof.

A compound of Formula (I), or a pharmaceutically acceptable salt thereof, can have one of the following structures:

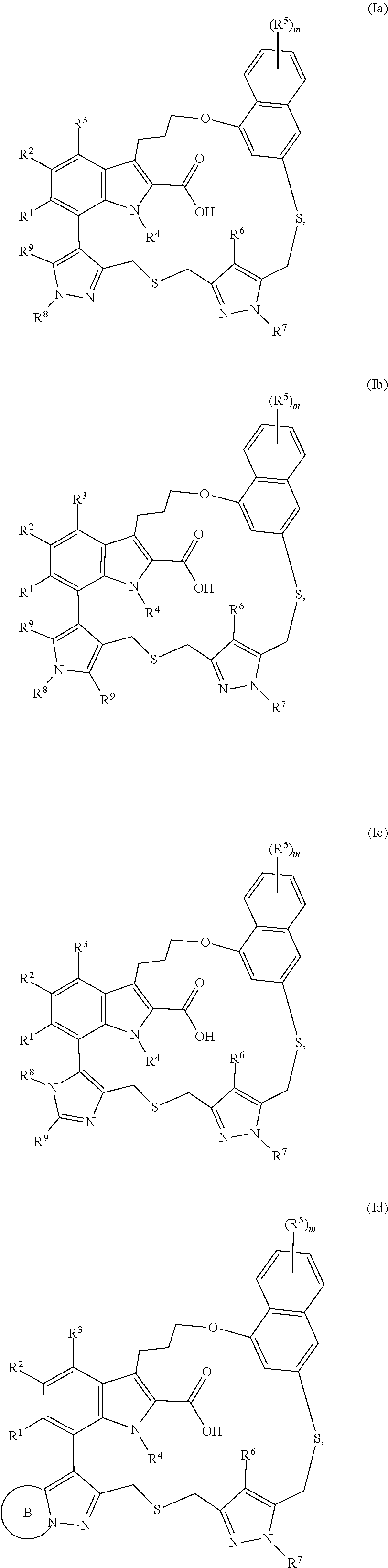

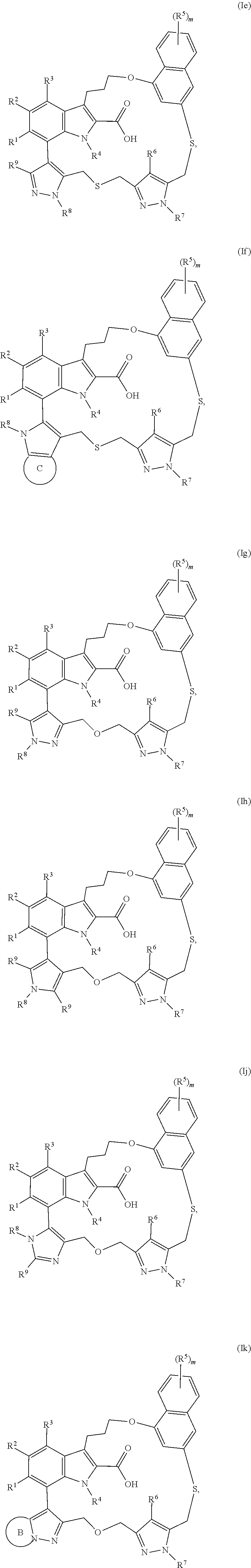

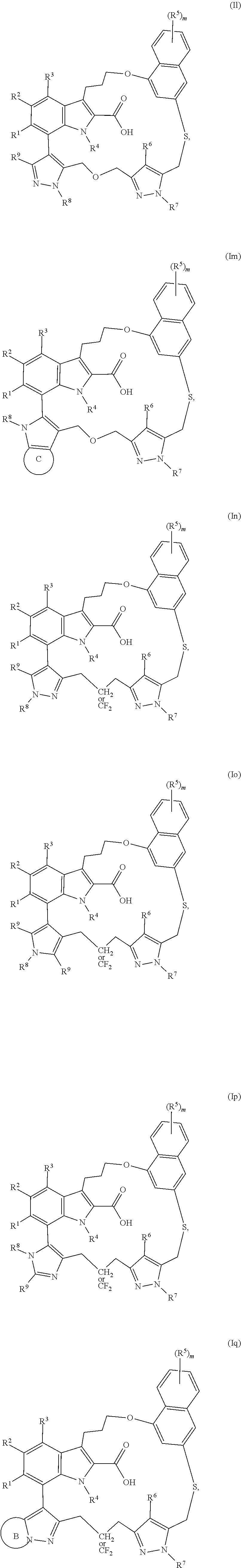

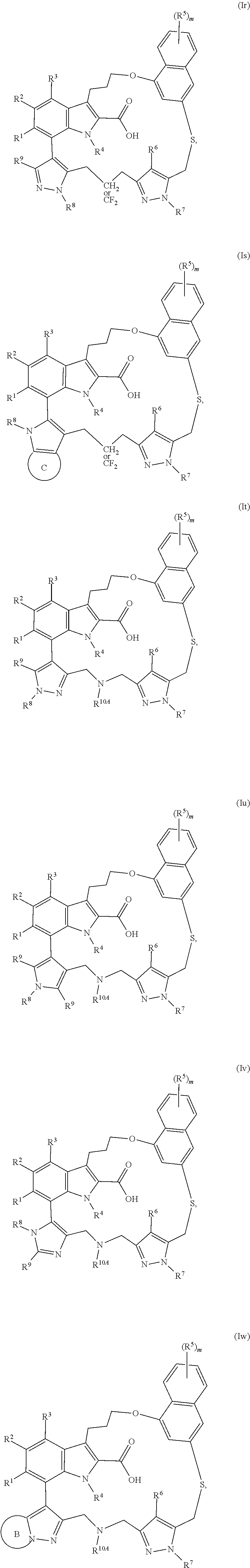

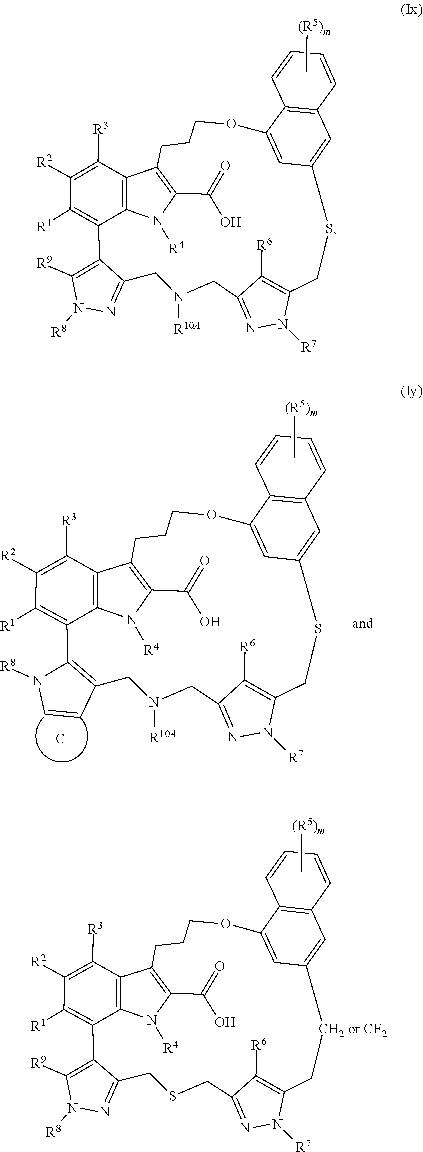

(Iz), including pharmaceutically acceptable salts of any of the foregoing.

Examples of compounds of Formula (I), and pharmaceutically acceptable salts thereof, include the following:

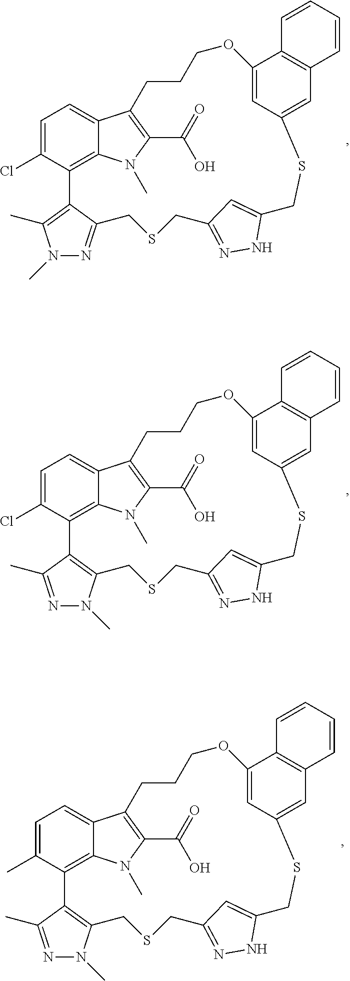 [SMILES: Cc1c2c(nn1C)CSCc1cc([nH]n1)CSc1cc(c3ccccc3c1)OCCCc1c(C(=O)O)n(C)c3c-2c(Cl)ccc13 ; Cc1nn(C)c2c1-c1c(Cl)ccc3c(c(C(=O)O)n(C)c13)CCCOc1cc(cc3ccccc13)SCc1cc(n[nH]1)CSC2 ; Cc1ccc2c3c(C(=O)O)n(C)c2c1-c1c(C)nn(C)c1CSCc1cc([nH]n1)CSc1cc(c2ccccc2c1)OCCC3]

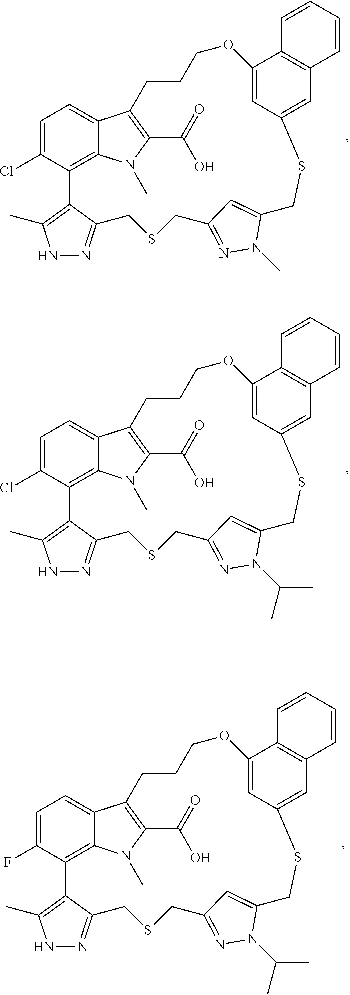 [SMILES: Cc1[nH]nc2c1-c1c(Cl)ccc3c(c(C(=O)O)n(C)c13)CCCOc1cc(cc3ccccc13)SCc1cc(nn1C)CSC2 ; Cc1[nH]nc2c1-c1c(Cl)ccc3c(c(C(=O)O)n(C)c13)CCCOc1cc(cc3ccccc13)SCc1cc(nn1C(C)C)CSC2 ; Cc1[nH]nc2c1-c1c(F)ccc3c(c(C(=O)O)n(C)c13)CCCOc1cc(cc3ccccc13)SCc1cc(nn1C(C)C)CSC2]

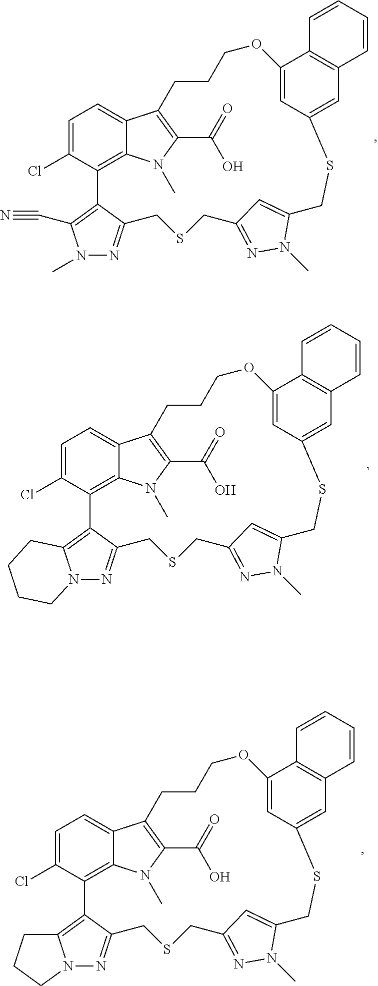 [SMILES: Cn1nc2cc1CSc1cc(c3ccccc3c1)OCCCc1c(C(=O)O)n(C)c3c(c(Cl)ccc13)-c1c(nn(C)c1C#N)CSC2 ; Cn1nc2cc1CSc1cc(c3ccccc3c1)OCCCc1c(C(=O)O)n(C)c3c(c(Cl)ccc13)-c1c(nn3c1CCCC3)CSC2 ; Cn1nc2cc1CSc1cc(c3ccccc3c1)OCCCc1c(C(=O)O)n(C)c3c(c(Cl)ccc13)-c1c(nn3c1CCC3)CSC2]

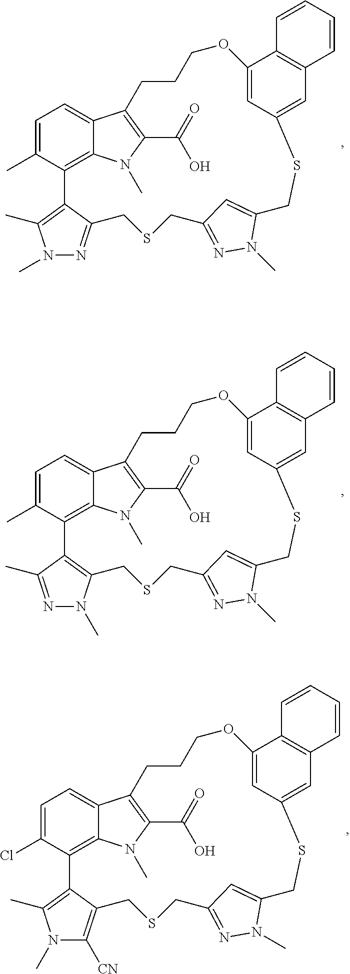 [SMILES: Cc1ccc2c3c(C(=O)O)n(C)c2c1-c1c(nn(C)c1C)CSCc1cc(n(C)n1)CSc1cc(c2ccccc2c1)OCCC3 ; Cc1ccc2c3c(C(=O)O)n(C)c2c1-c1c(C)nn(C)c1CSCc1cc(n(C)n1)CSc1cc(c2ccccc2c1)OCCC3 ; Cc1c2c(c(C#N)n1C)CSCc1cc(n(C)n1)CSc1cc(c3ccccc3c1)OCCCc1c(C(=O)O)n(C)c3c-2c(Cl)ccc13]

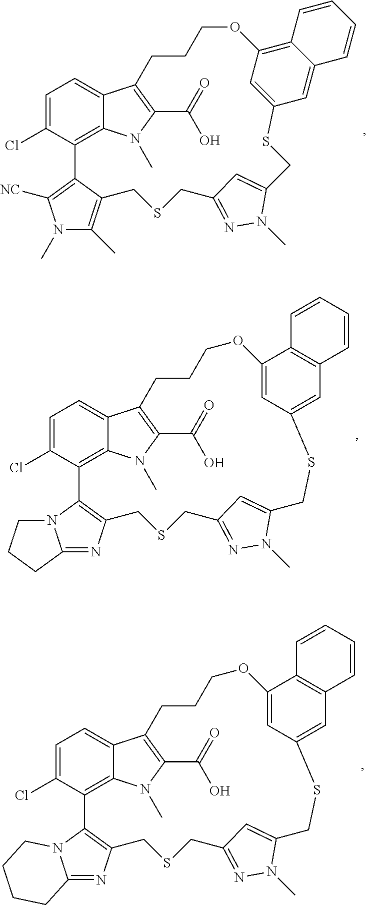 [SMILES: Cc1c2c(c(C#N)n1C)-c1c(Cl)ccc3c(c(C(=O)O)n(C)c13)CCCOc1cc(cc3ccccc13)SCc1cc(nn1C)CSC2 ; Cn1nc2cc1CSc1cc(c3ccccc3c1)OCCCc1c(C(=O)O)n(C)c3c(c(Cl)ccc13)-c1c(nc3n1CCC3)CSC2 ; Cn1nc2cc1CSc1cc(c3ccccc3c1)OCCCc1c(C(=O)O)n(C)c3c(c(Cl)ccc13)-c1c(nc3n1CCCC3)CSC2]

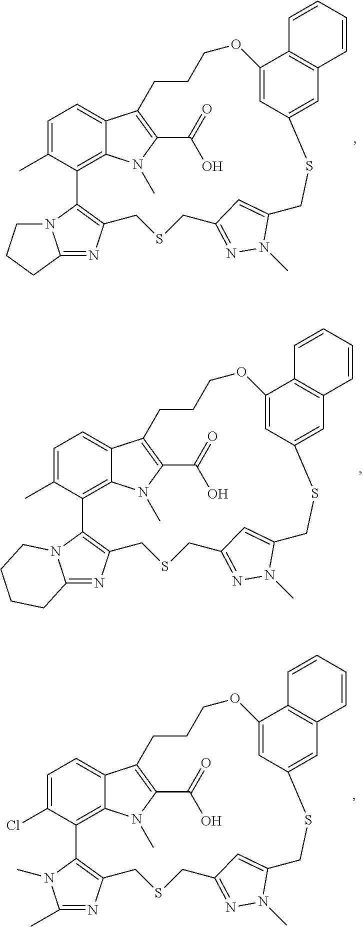 [SMILES: Cc1ccc2c3c(C(=O)O)n(C)c2c1-c1c(nc2n1CCC2)CSCc1cc(n(C)n1)CSc1cc(c2ccccc2c1)OCCC3 ; Cc1ccc2c3c(C(=O)O)n(C)c2c1-c1c(nc2n1CCCC2)CSCc1cc(n(C)n1)CSc1cc(c2ccccc2c1)OCCC3 ; Cc1nc2c(n1C)-c1c(Cl)ccc3c(c(C(=O)O)n(C)c13)CCCOc1cc(cc3ccccc13)SCc1cc(nn1C)CSC2]

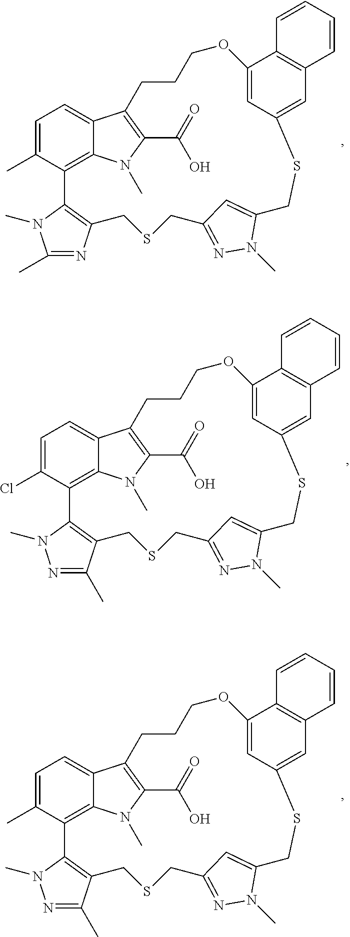 [SMILES: Cc1ccc2c3c(C(=O)O)n(C)c2c1-c1c(nc(C)n1C)CSCc1cc(n(C)n1)CSc1cc(c2ccccc2c1)OCCC3 ; Cc1nn(C)c2c1CSCc1cc(n(C)n1)CSc1cc(c3ccccc3c1)OCCCc1c(C(=O)O)n(C)c3c-2c(Cl)ccc13 ; Cc1ccc2c3c(C(=O)O)n(C)c2c1-c1c(c(C)nn1C)CSCc1cc(n(C)n1)CSc1cc(c2ccccc2c1)OCCC3]

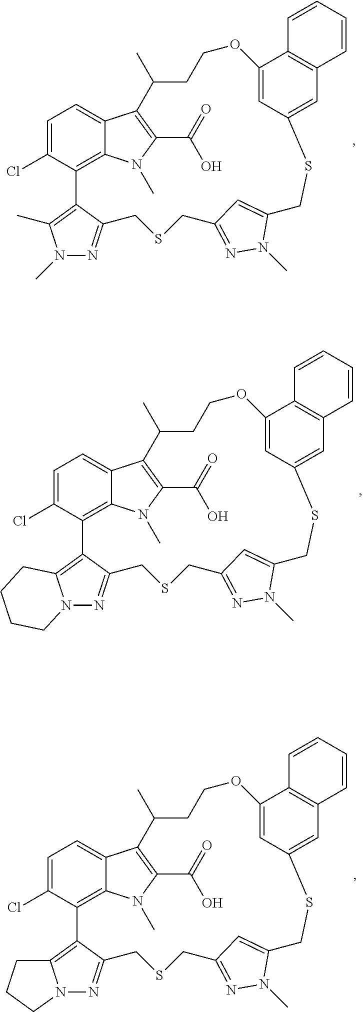 [SMILES: Cc1c2c(nn1C)CSCc1cc(n(C)n1)CSc1cc(c3ccccc3c1)OCCC(C)c1c(C(=O)O)n(C)c3c-2c(Cl)ccc13 ; CC1CCOc2cc(cc3ccccc23)SCc2cc(nn2C)CSCc2nn3c(c2-c2c(Cl)ccc4c1c(C(=O)O)n(C)c24)CCCC3 ; CC1CCOc2cc(cc3ccccc23)SCc2cc(nn2C)CSCc2nn3c(c2-c2c(Cl)ccc4c1c(C(=O)O)n(C)c24)CCC3]

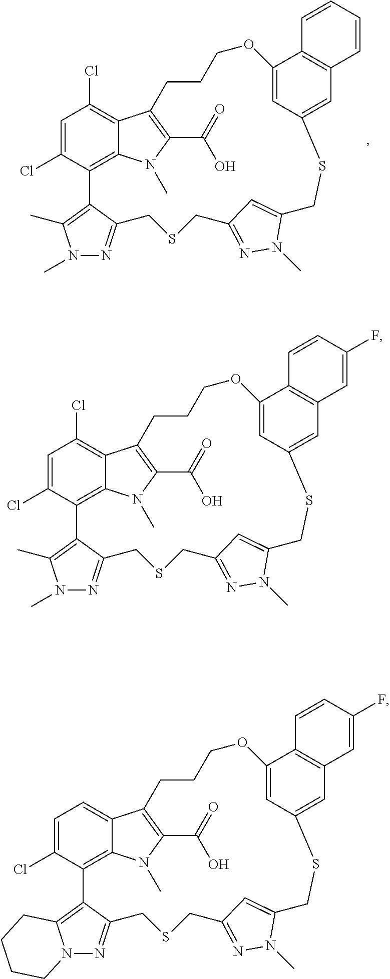 [SMILES: Cc1c2c(nn1C)CSCc1cc(n(C)n1)CSc1cc(c3ccccc3c1)OCCCc1c(C(=O)O)n(C)c3c-2c(Cl)cc(Cl)c13 ; Cc1c2c(nn1C)CSCc1cc(n(C)n1)CSc1cc(c3ccc(F)cc3c1)OCCCc1c(C(=O)O)n(C)c3c-2c(Cl)cc(Cl)c13 ; Cn1nc2cc1CSc1cc(c3ccc(F)cc3c1)OCCCc1c(C(=O)O)n(C)c3c(c(Cl)ccc13)-c1c(nn3c1CCCC3)CSC2]

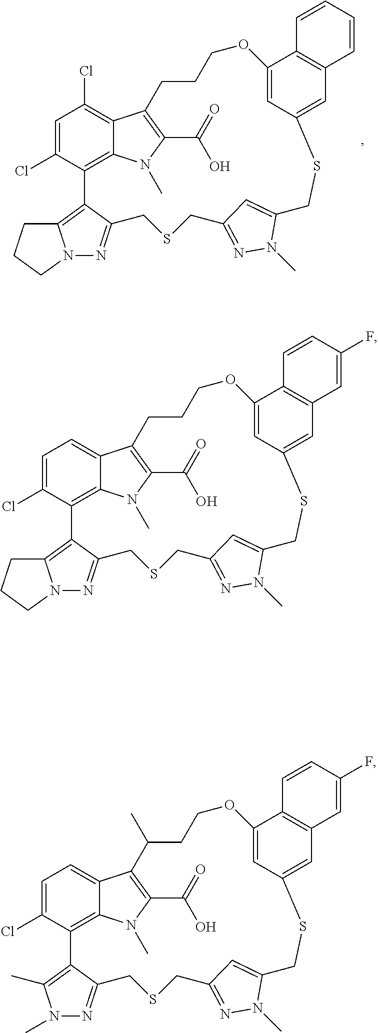 [SMILES: Cn1nc2cc1CSc1cc(c3ccccc3c1)OCCCc1c(C(=O)O)n(C)c3c(c(Cl)cc(Cl)c13)-c1c(nn3c1CCC3)CSC2 ; Cn1nc2cc1CSc1cc(c3ccc(F)cc3c1)OCCCc1c(C(=O)O)n(C)c3c(c(Cl)ccc13)-c1c(nn3c1CCC3)CSC2 ; Cc1c2c(nn1C)CSCc1cc(n(C)n1)CSc1cc(c3ccc(F)cc3c1)OCCC(C)c1c(C(=O)O)n(C)c3c-2c(Cl)ccc13]

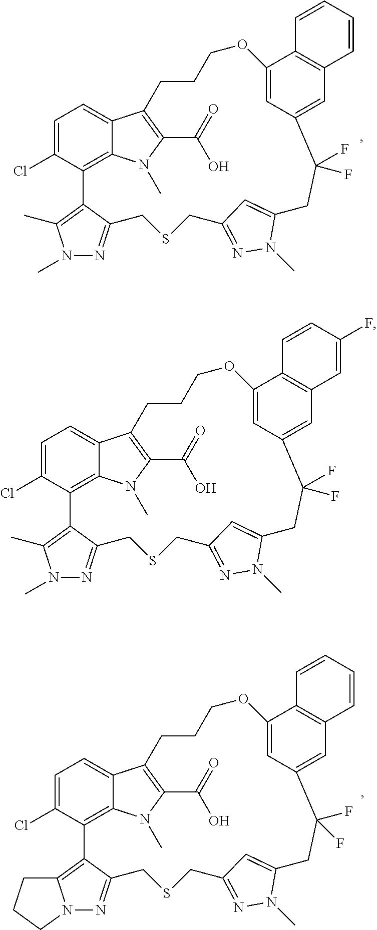 [SMILES: Cc1c2c(nn1C)CSCc1cc(n(C)n1)CC(F)(F)c1cc(c3ccccc3c1)OCCCc1c(C(=O)O)n(C)c3c-2c(Cl)ccc13 ; Cc1c2c(nn1C)CSCc1cc(n(C)n1)CC(F)(F)c1cc(c3ccc(F)cc3c1)OCCCc1c(C(=O)O)n(C)c3c-2c(Cl)ccc13 ; Cn1nc2cc1CC(F)(F)c1cc(c3ccccc3c1)OCCCc1c(C(=O)O)n(C)c3c(c(Cl)ccc13)-c1c(nn3c1CCC3)CSC2]

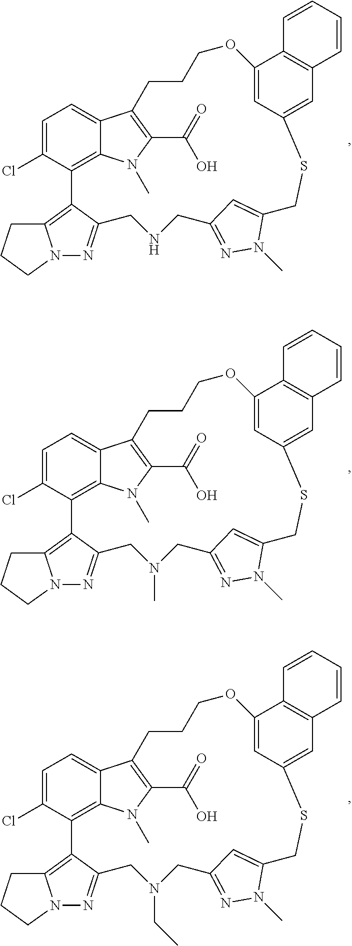 [SMILES: Cn1nc2cc1CSc1cc(c3ccccc3c1)OCCCc1c(C(=O)O)n(C)c3c(c(Cl)ccc13)-c1c(nn3c1CCC3)CNC2 ; CN1Cc2cc(n(C)n2)CSc2cc(c3ccccc3c2)OCCCc2c(C(=O)O)n(C)c3c(c(Cl)ccc23)-c2c(nn3c2CCC3)C1 ; CCN1Cc2cc(n(C)n2)CSc2cc(c3ccccc3c2)OCCCc2c(C(=O)O)n(C)c3c(c(Cl)ccc23)-c2c(nn3c2CCC3)C1]

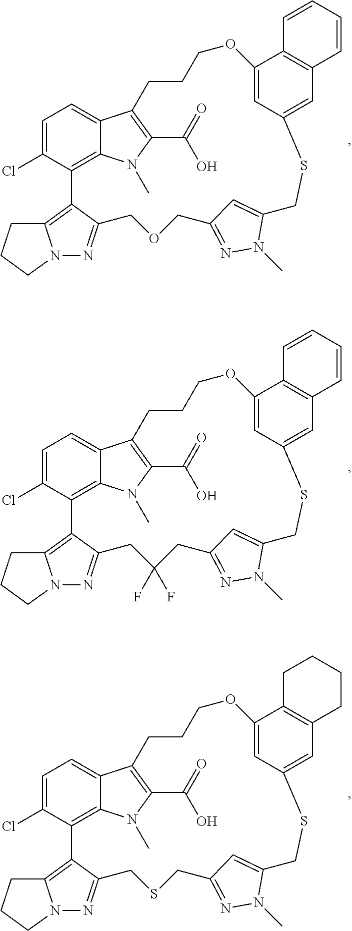 [SMILES: Cn1nc2cc1CSc1cc(c3ccccc3c1)OCCCc1c(C(=O)O)n(C)c3c(c(Cl)ccc13)-c1c(nn3c1CCC3)COC2 ; Cn1nc2cc1CSc1cc(c3ccccc3c1)OCCCc1c(C(=O)O)n(C)c3c(c(Cl)ccc13)-c1c(nn3c1CCC3)CC(F)(F)C2 ; Cn1nc2cc1CSc1cc3c(c(c1)OCCCc1c(C(=O)O)n(C)c4c(c(Cl)ccc14)-c1c(nn4c1CCC4)CSC2)CCCC3]

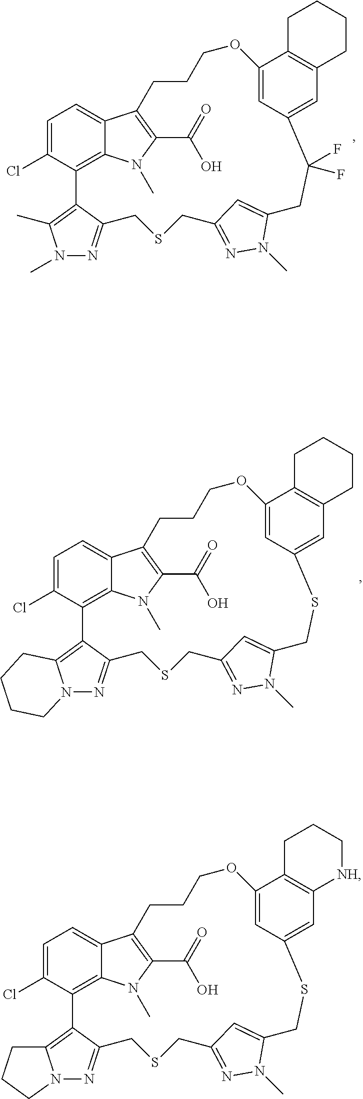 [SMILES: Cc1c2c(nn1C)CSCc1cc(n(C)n1)CC(F)(F)c1cc3c(c(c1)OCCCc1c(C(=O)O)n(C)c4c-2c(Cl)ccc14)CCCC3 ; Cn1nc2cc1CSc1cc3c(c(c1)OCCCc1c(C(=O)O)n(C)c4c(c(Cl)ccc14)-c1c(nn4c1CCCC4)CSC2)CCCC3 ; Cn1nc2cc1CSc1cc3c(c(c1)OCCCc1c(C(=O)O)n(C)c4c(c(Cl)ccc14)-c1c(nn4c1CCC4)CSC2)CCCN3]

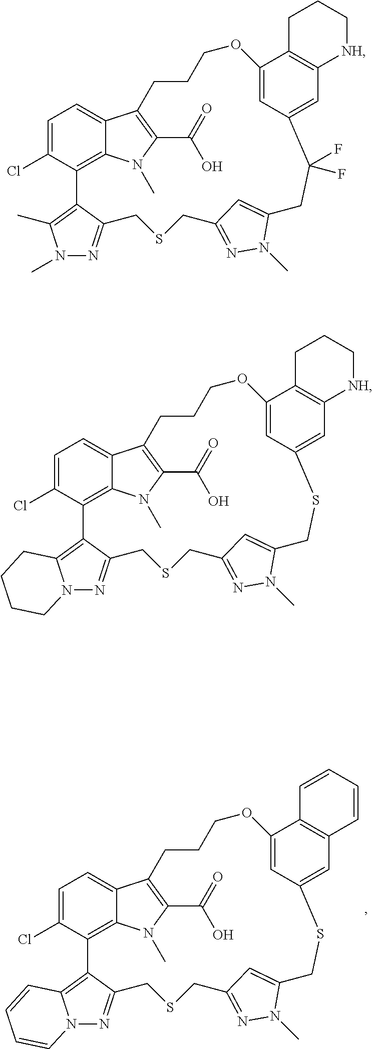 [SMILES: Cc1c2c(nn1C)CSCc1cc(n(C)n1)CC(F)(F)c1cc3c(c(c1)OCCCc1c(C(=O)O)n(C)c4c-2c(Cl)ccc14)CCCN3 ; Cn1nc2cc1CSc1cc3c(c(c1)OCCCc1c(C(=O)O)n(C)c4c(c(Cl)ccc14)-c1c(nn4c1CCCC4)CSC2)CCCN3 ; Cn1nc2cc1CSc1cc(c3ccccc3c1)OCCCc1c(C(=O)O)n(C)c3c(c(Cl)ccc13)-c1c(nn3ccccc13)CSC2]

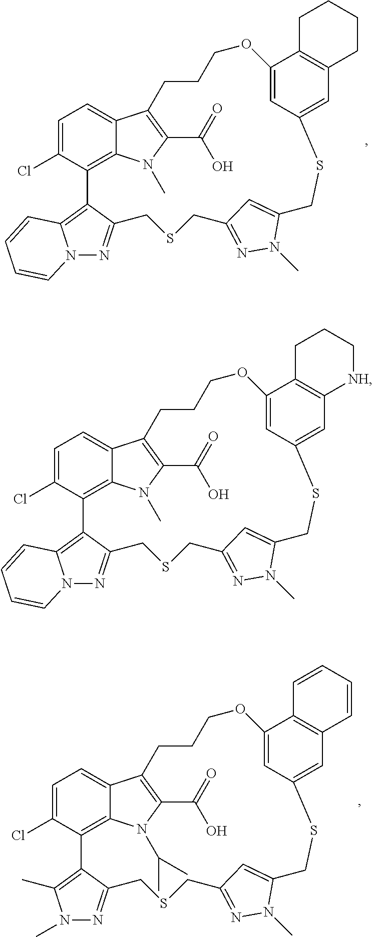 [SMILES: Cn1nc2cc1CSc1cc3c(c(c1)OCCCc1c(C(=O)O)n(C)c4c(c(Cl)ccc14)-c1c(nn4ccccc14)CSC2)CCCC3 ; Cn1nc2cc1CSc1cc3c(c(c1)OCCCc1c(C(=O)O)n(C)c4c(c(Cl)ccc14)-c1c(nn4ccccc14)CSC2)CCCN3 ; Cc1c2c(nn1C)CSCc1cc(n(C)n1)CSc1cc(c3ccccc3c1)OCCCc1c(C(=O)O)n(C(C)C)c3c-2c(Cl)ccc13]

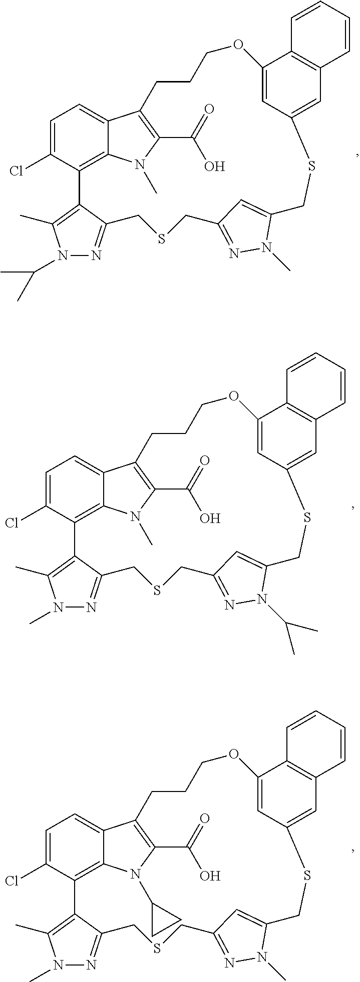 [SMILES: Cc1c2c(nn1C(C)C)CSCc1cc(n(C)n1)CSc1cc(c3ccccc3c1)OCCCc1c(C(=O)O)n(C)c3c-2c(Cl)ccc13 ; Cc1c2c(nn1C)CSCc1cc(n(C(C)C)n1)CSc1cc(c3ccccc3c1)OCCCc1c(C(=O)O)n(C)c3c-2c(Cl)ccc13 ; Cc1c2c(nn1C)CSCc1cc(n(C)n1)CSc1cc(c3ccccc3c1)OCCCc1c(C(=O)O)n(C3CC3)c3c-2c(Cl)ccc13]

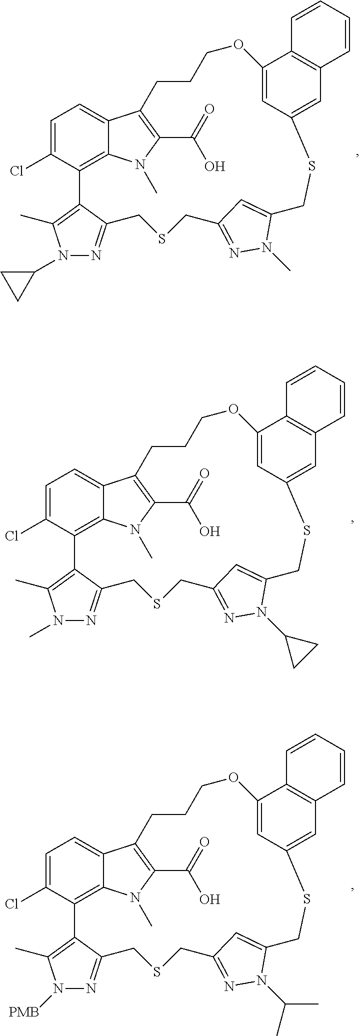

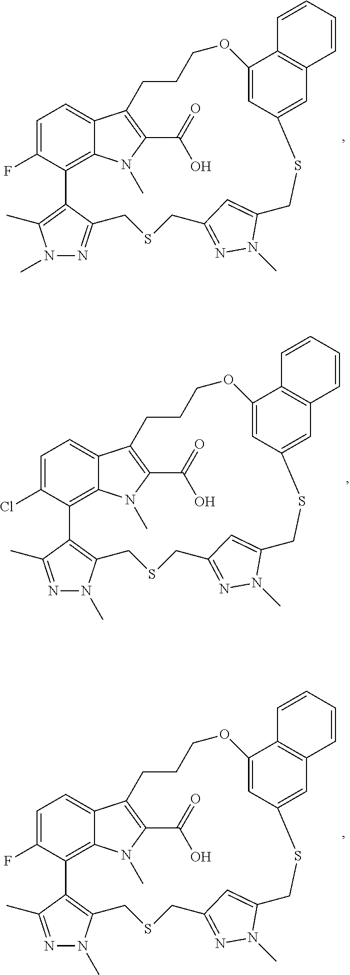 [SMILES: Cc1c2c(nn1C)CSCc1cc(n(C)n1)CSc1cc(c3ccccc3c1)OCCCc1c(C(=O)O)n(C)c3c-2c(F)ccc13 ; Cc1nn(C)c2c1-c1c(Cl)ccc3c(c(C(=O)O)n(C)c13)CCCOc1cc(cc3ccccc13)SCc1cc(nn1C)CSC2 ; Cc1nn(C)c2c1-c1c(F)ccc3c(c(C(=O)O)n(C)c13)CCCOc1cc(cc3ccccc13)SCc1cc(nn1C)CSC2]

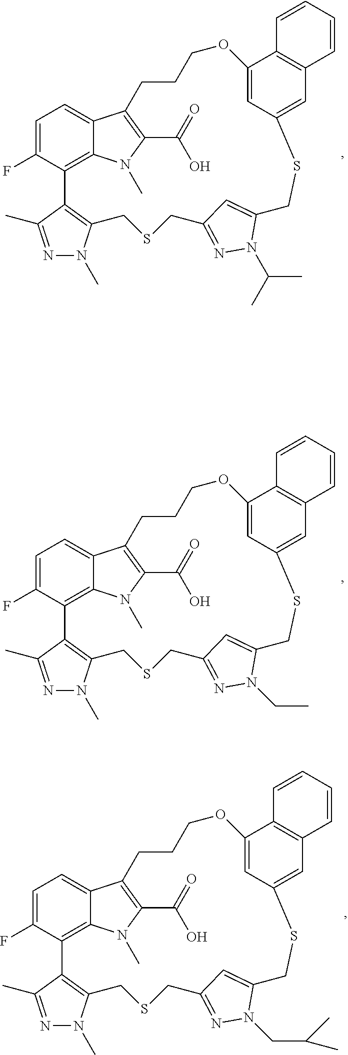 [SMILES: Cc1nn(C)c2c1-c1c(F)ccc3c(c(C(=O)O)n(C)c13)CCCOc1cc(cc3ccccc13)SCc1cc(nn1C(C)C)CSC2 ; CCn1nc2cc1CSc1cc(c3ccccc3c1)OCCCc1c(C(=O)O)n(C)c3c(c(F)ccc13)-c1c(C)nn(C)c1CSC2 ; Cc1nn(C)c2c1-c1c(F)ccc3c(c(C(=O)O)n(C)c13)CCCOc1cc(cc3ccccc13)SCc1cc(nn1CC(C)C)CSC2]

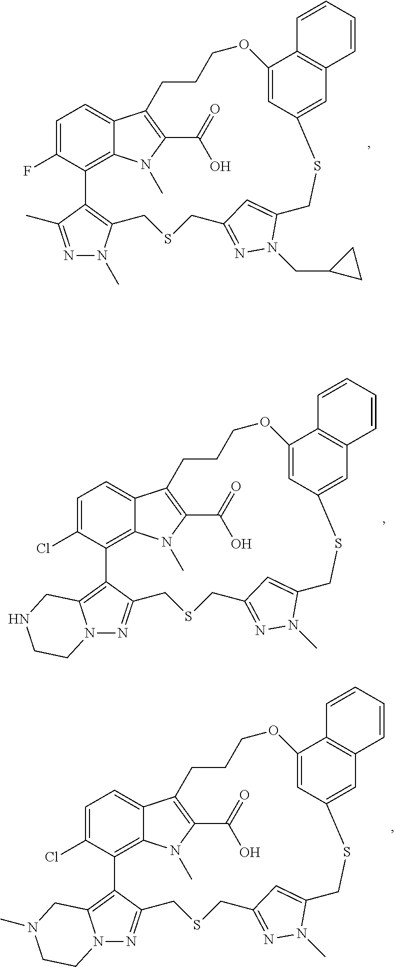 [SMILES: Cc1nn(C)c2c1-c1c(F)ccc3c(c(C(=O)O)n(C)c13)CCCOc1cc(cc3ccccc13)SCc1cc(nn1CC1CC1)CSC2 ; Cn1nc2cc1CSc1cc(c3ccccc3c1)OCCCc1c(C(=O)O)n(C)c3c(c(Cl)ccc13)-c1c(nn3c1CNCC3)CSC2 ; CN1CCn2nc3c(c2C1)-c1c(Cl)ccc2c(c(C(=O)O)n(C)c12)CCCOc1cc(cc2ccccc12)SCc1cc(nn1C)CSC3]

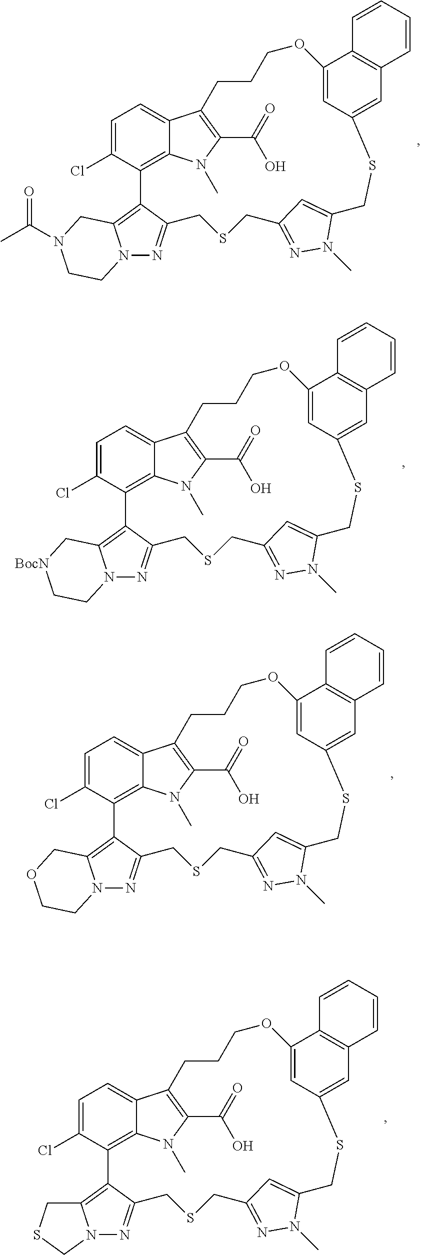

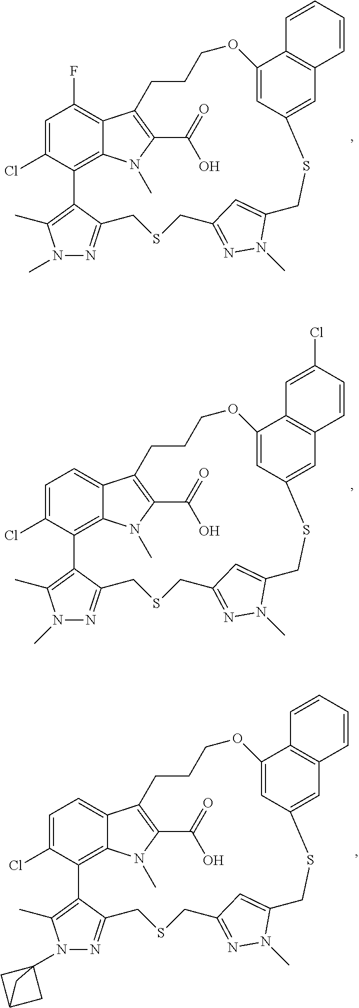 [SMILES: Cc1c2c(nn1C)CSCc1cc(n(C)n1)CSc1cc(c3ccccc3c1)OCCCc1c(C(=O)O)n(C)c3c-2c(Cl)cc(F)c13 ; Cc1c2c(nn1C)CSCc1cc(n(C)n1)CSc1cc(c3cc(Cl)ccc3c1)OCCCc1c(C(=O)O)n(C)c3c-2c(Cl)ccc13 ; Cc1c2c(nn1C13CC(C1)C3)CSCc1cc(n(C)n1)CSc1cc(c3ccccc3c1)OCCCc1c(C(=O)O)n(C)c3c-2c(Cl)ccc13]

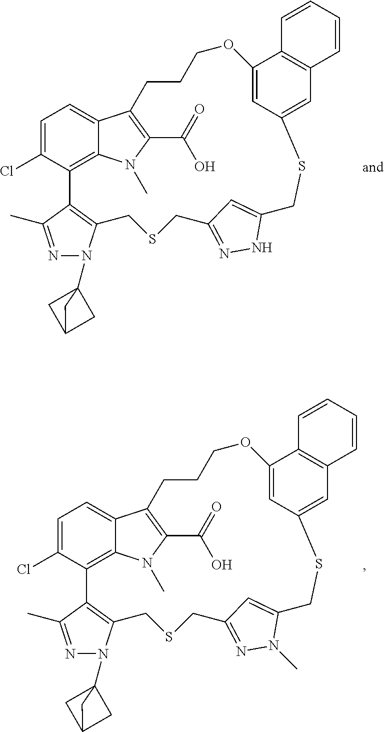 [SMILES: Cc1nn(C23CC(C2)C3)c2c1-c1c(Cl)ccc3c(c(C(=O)O)n(C)c13)CCCOc1cc(cc3ccccc13)SCc1cc(n[nH]1)CSC2 ; Cc1nn(C23CC(C2)C3)c2c1-c1c(Cl)ccc3c(c(C(=O)O)n(C)c13)CCCOc1cc(cc3ccccc13)SCc1cc(nn1C)CSC2]

or a pharmaceutically acceptable salt of any of the foregoing.

Additional examples of compounds of Formula (I), and pharmaceutically acceptable salts thereof, include the following:

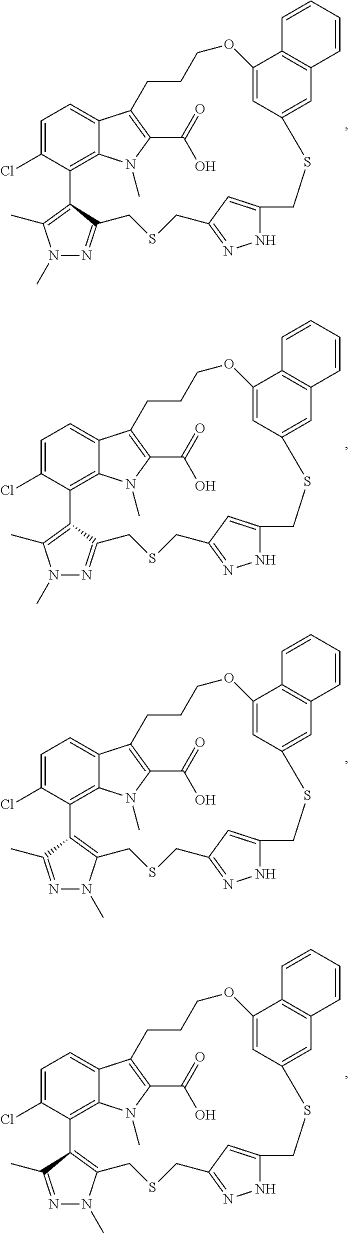

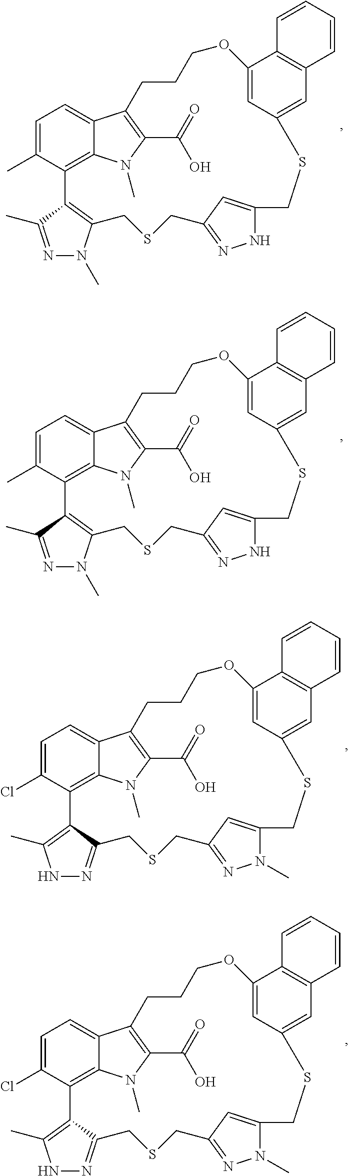

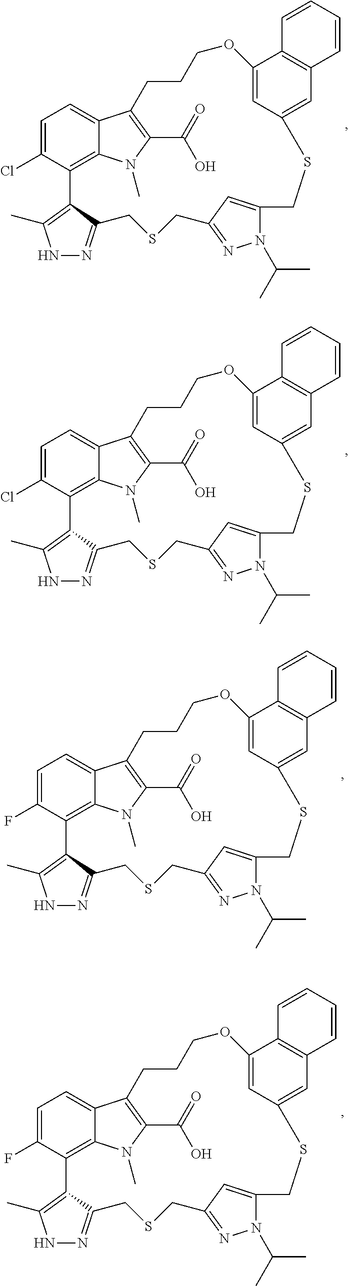

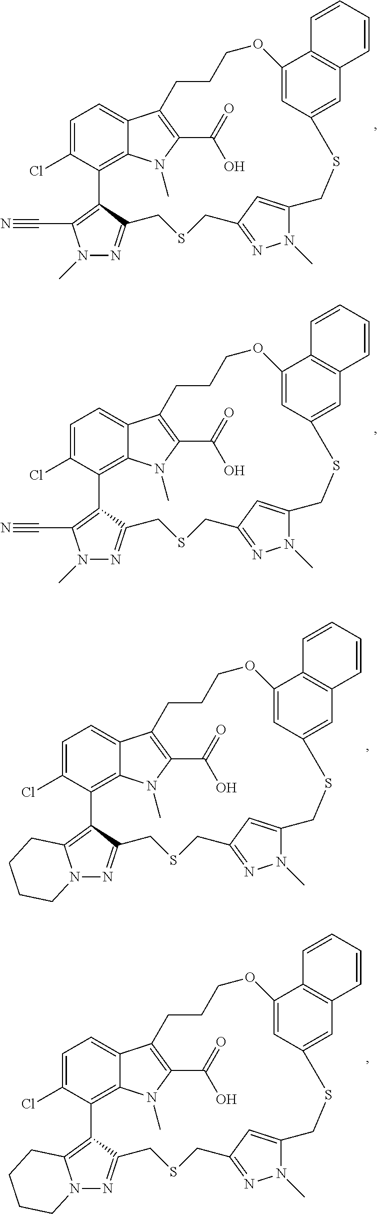

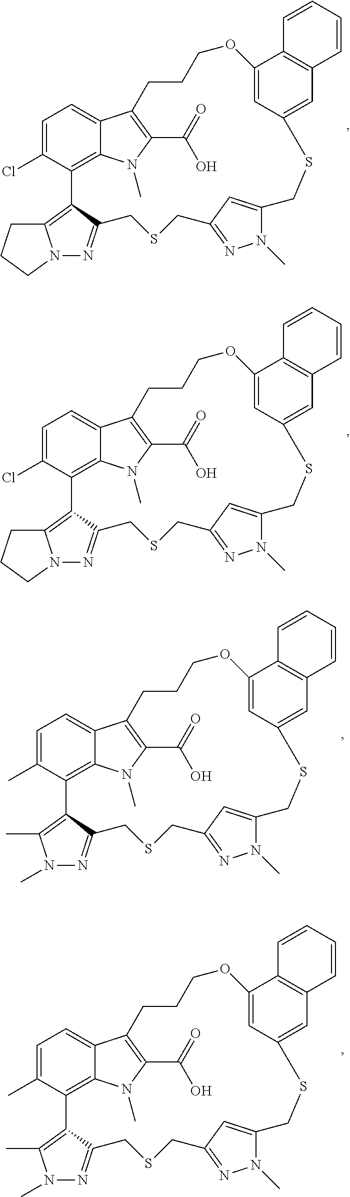

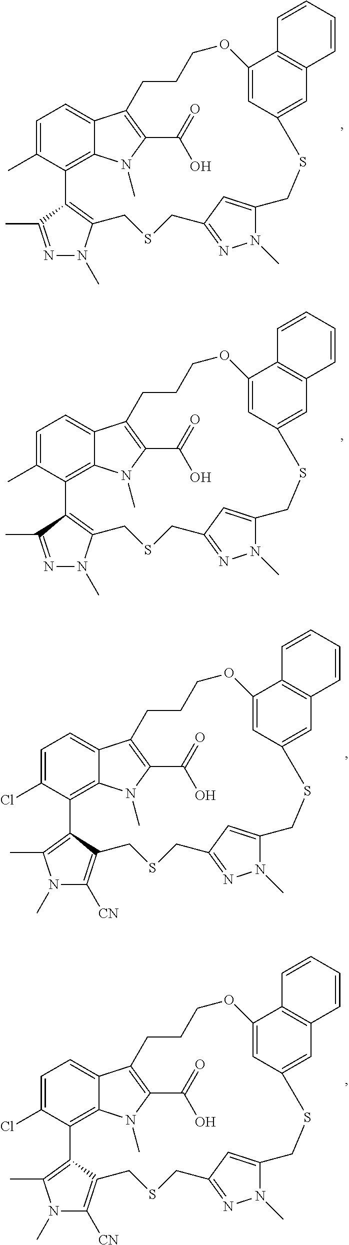

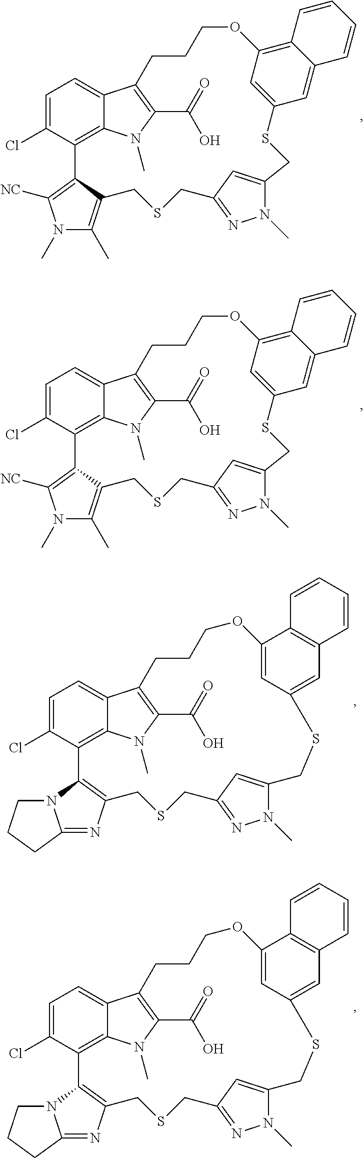

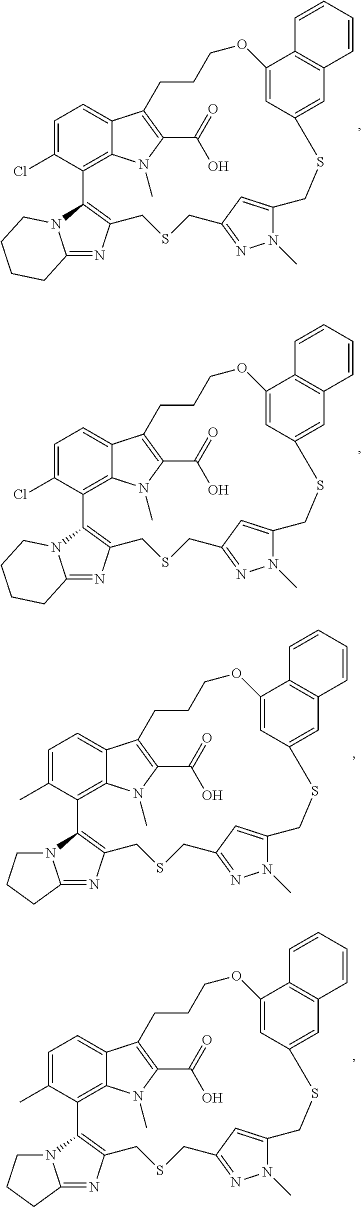

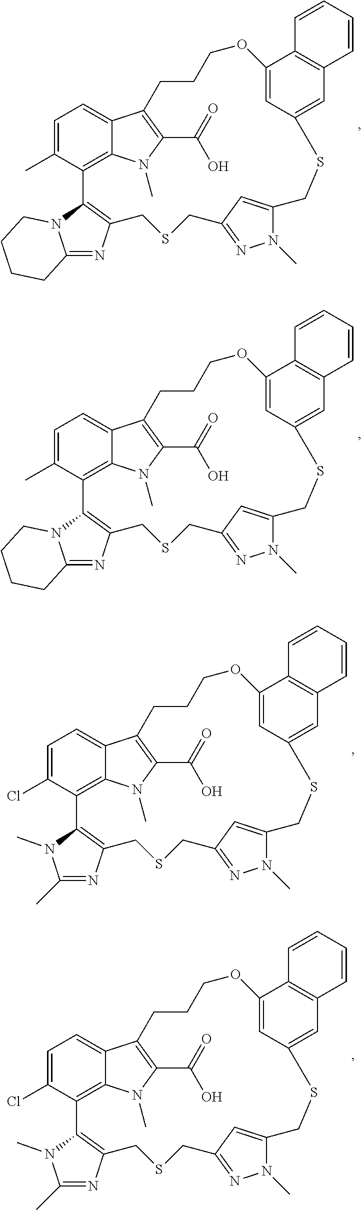

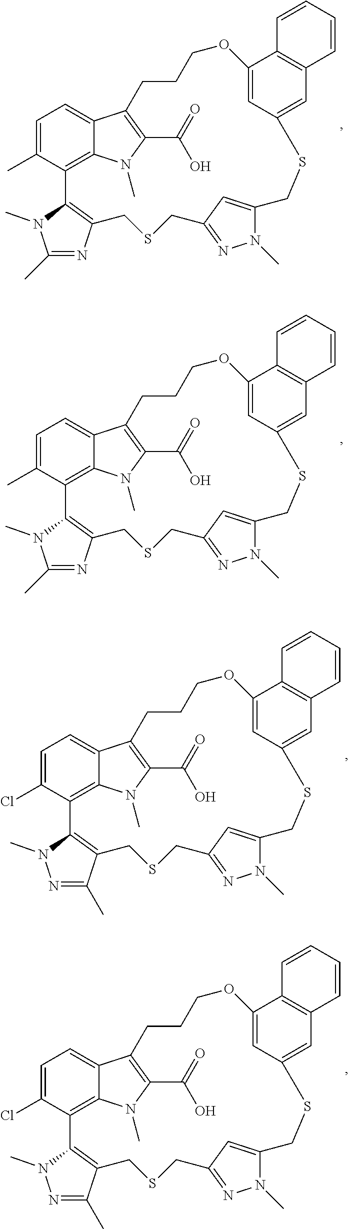

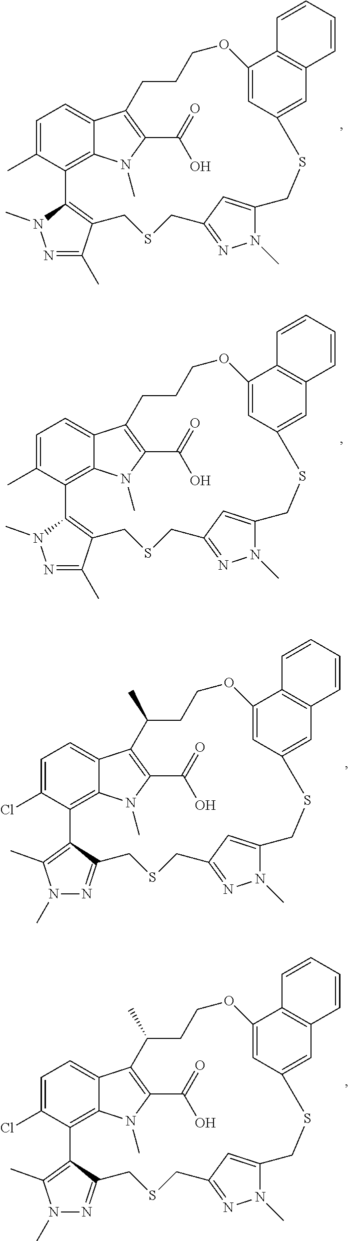

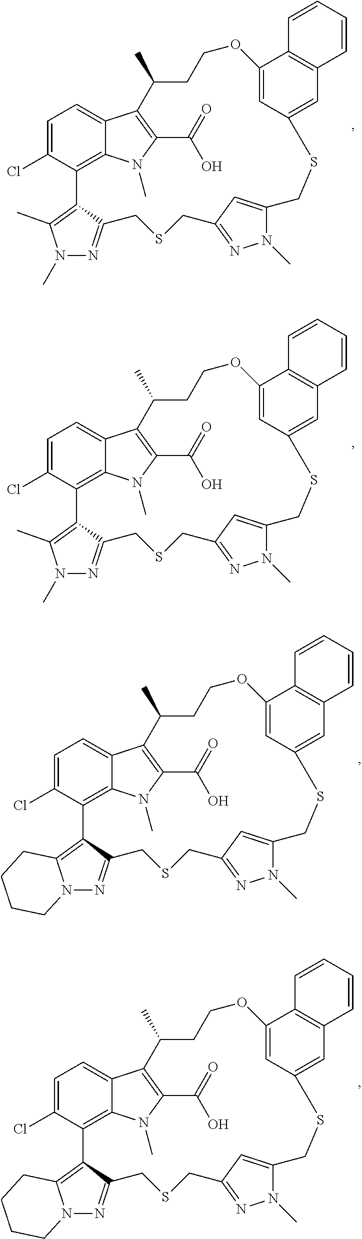

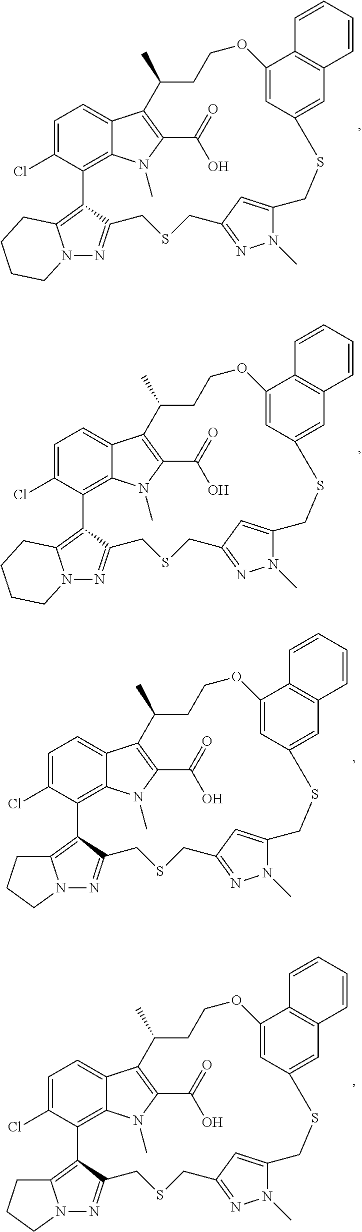

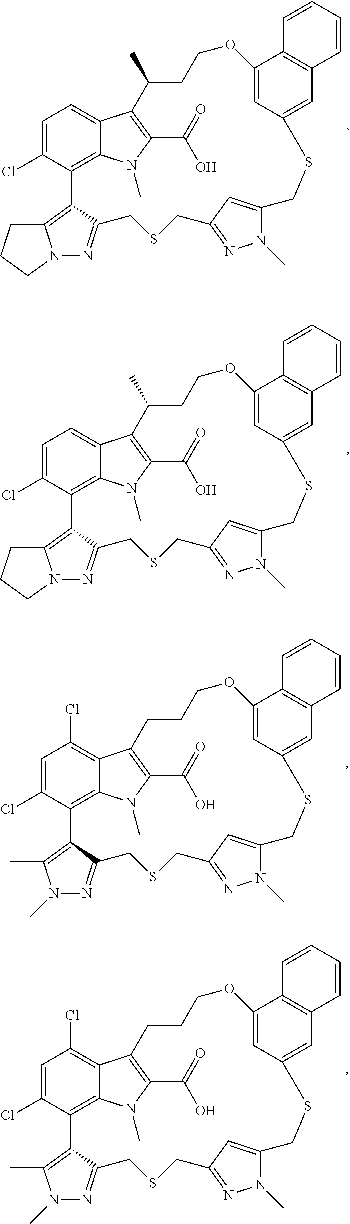

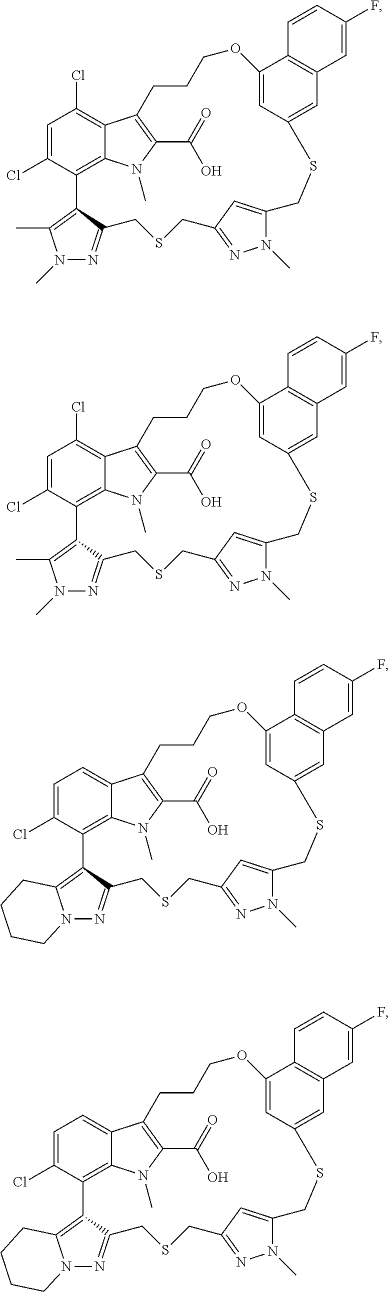

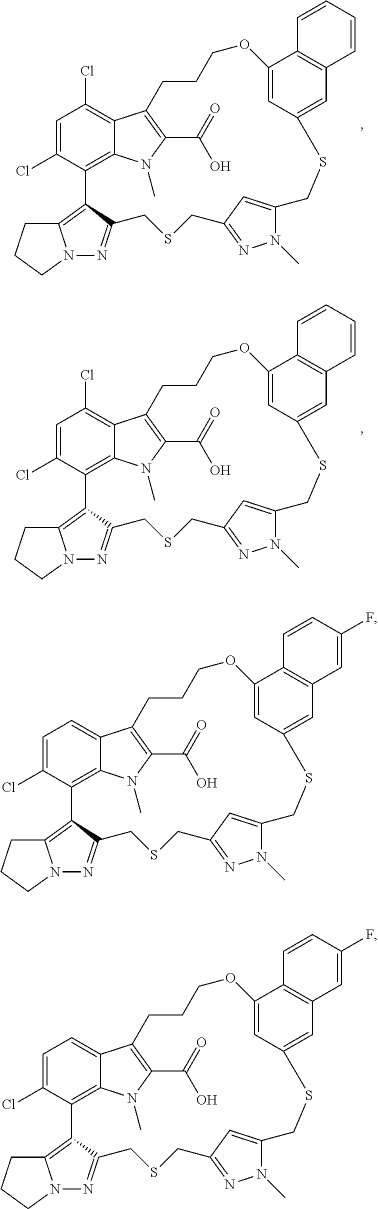

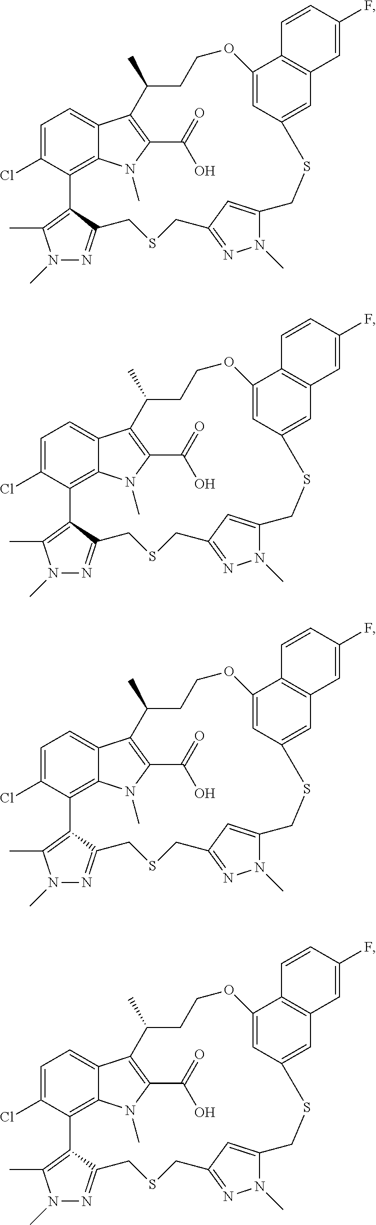

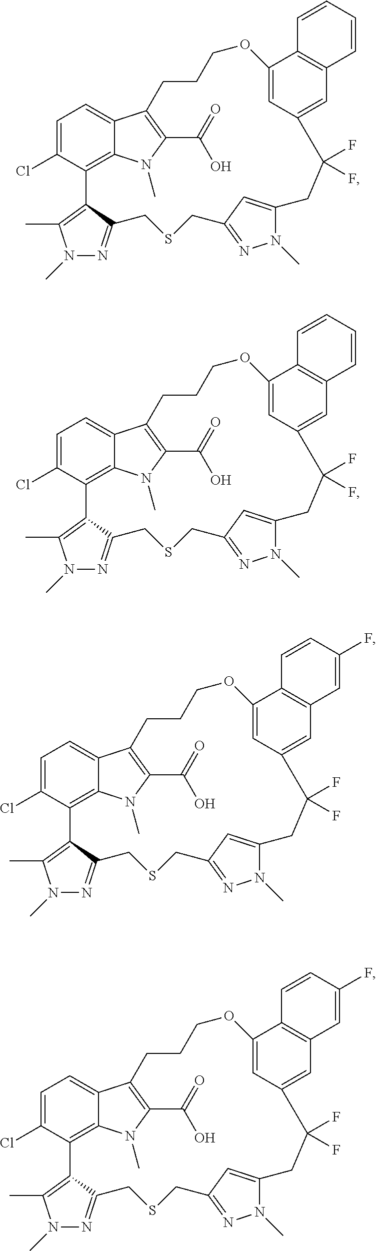

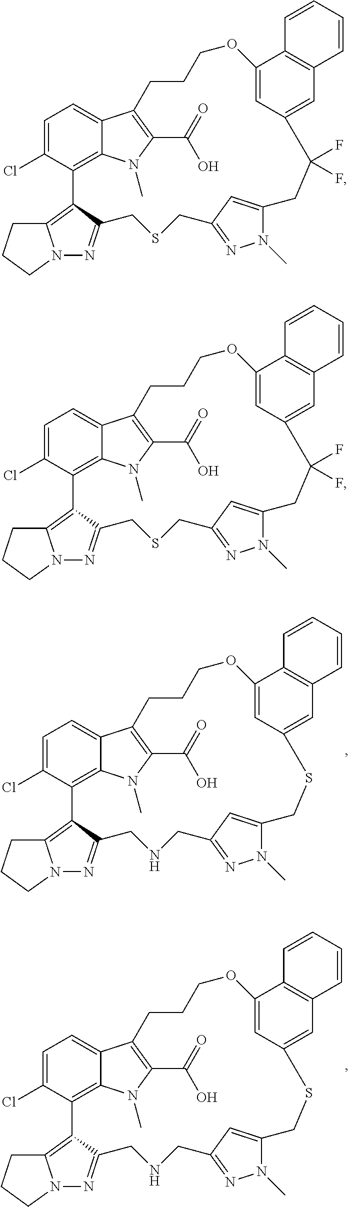

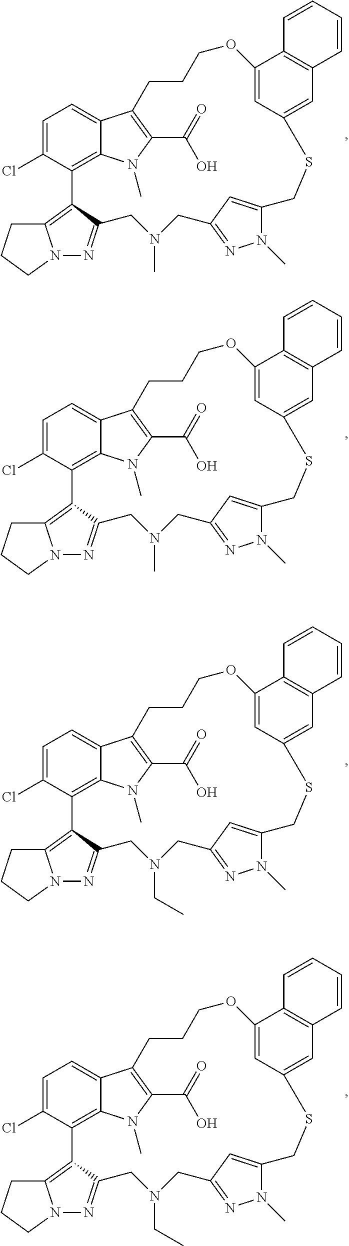

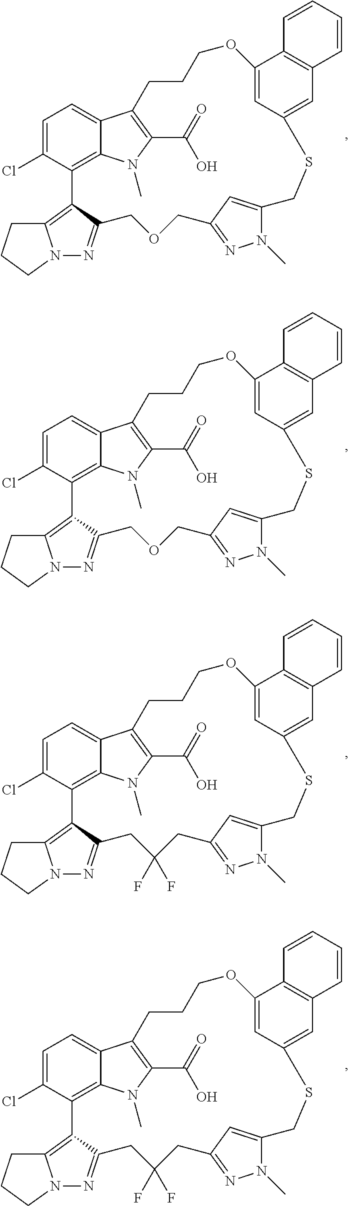

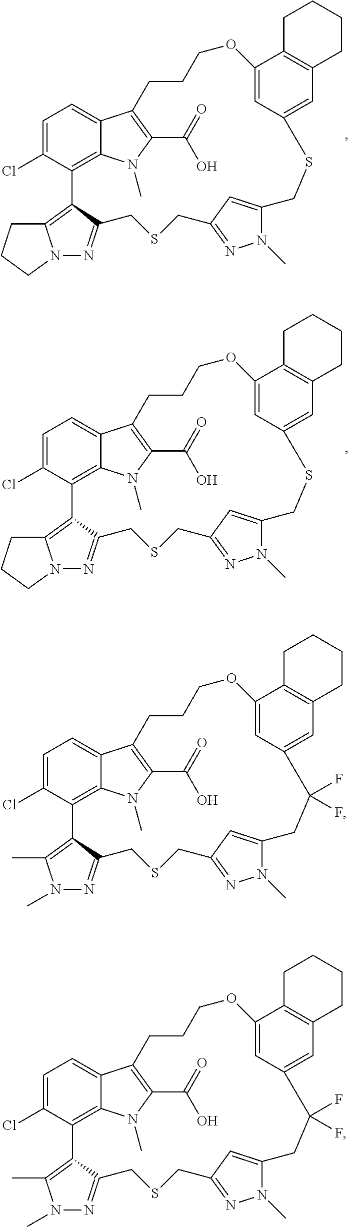

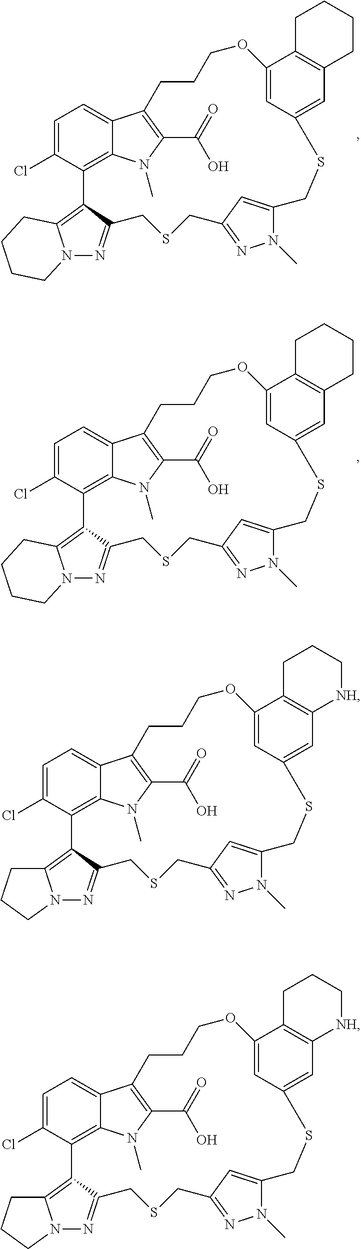

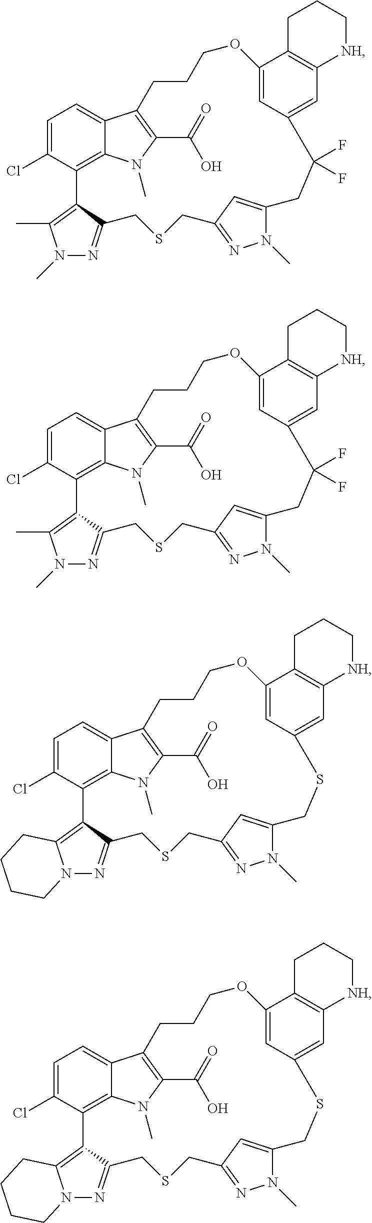

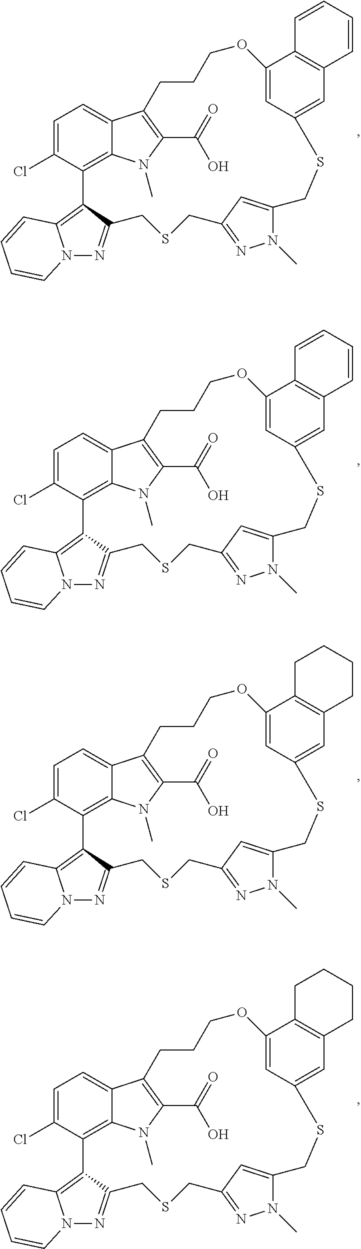

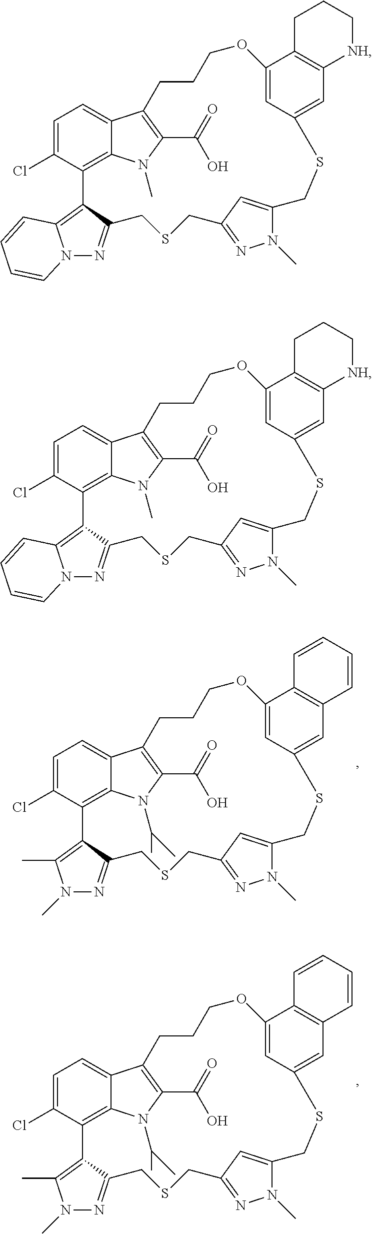

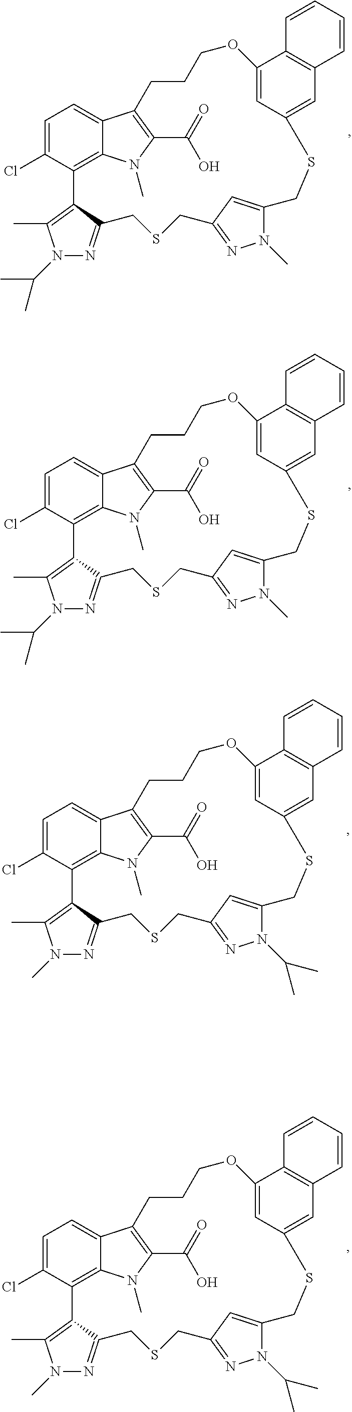

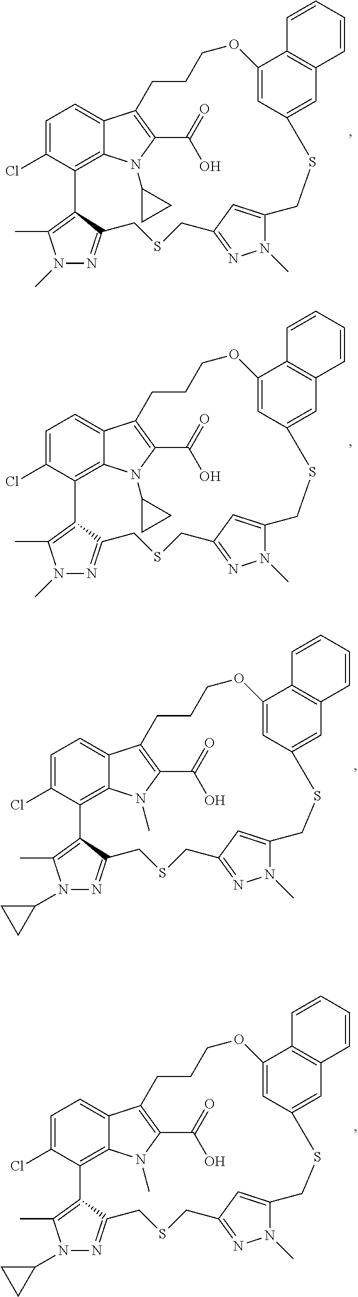

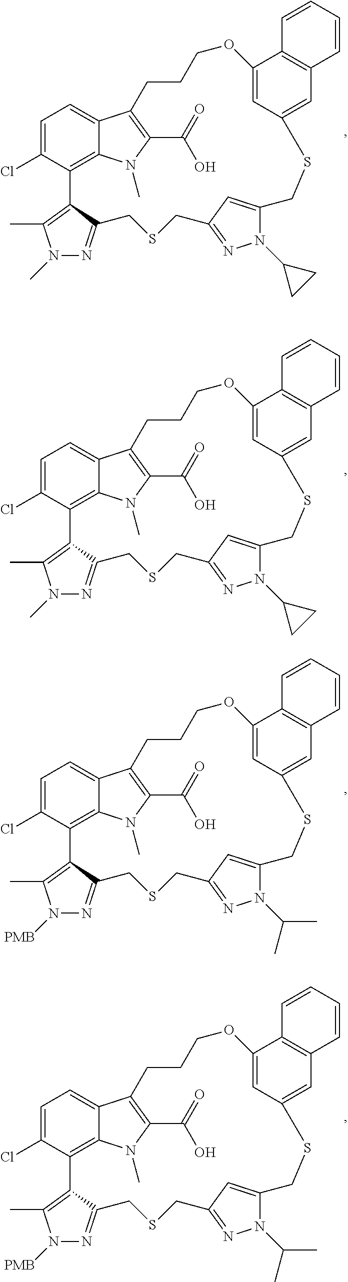

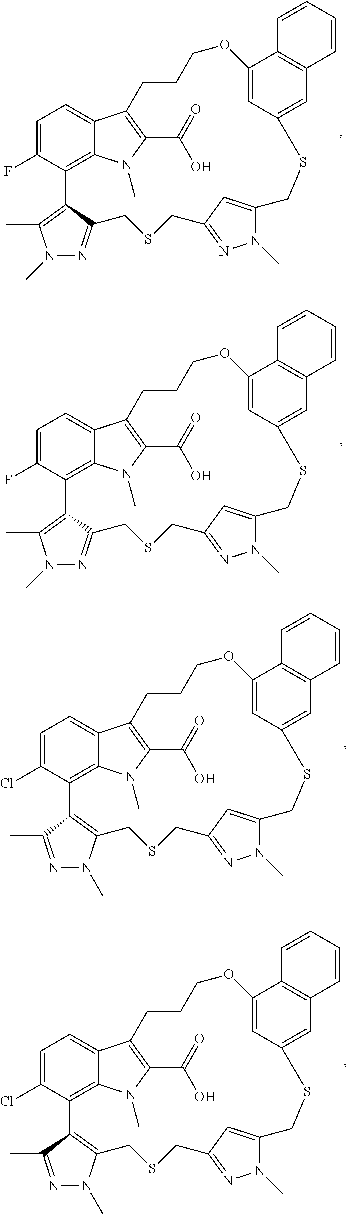

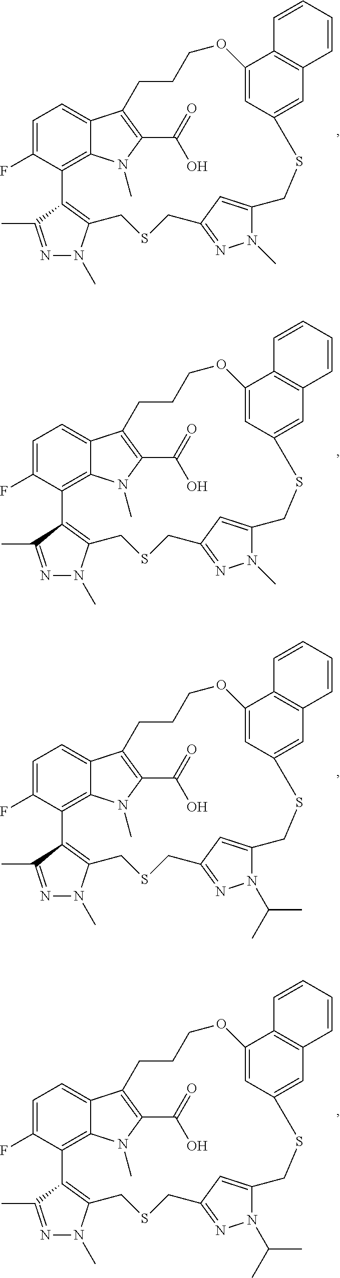

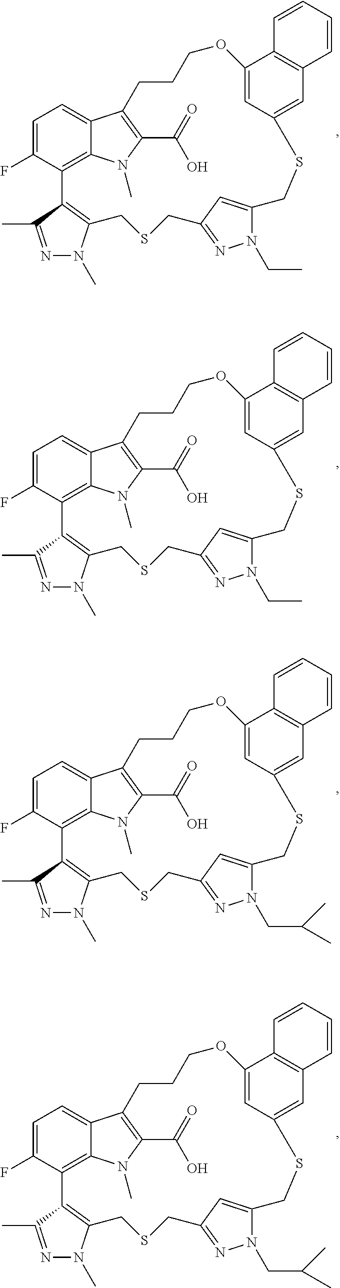

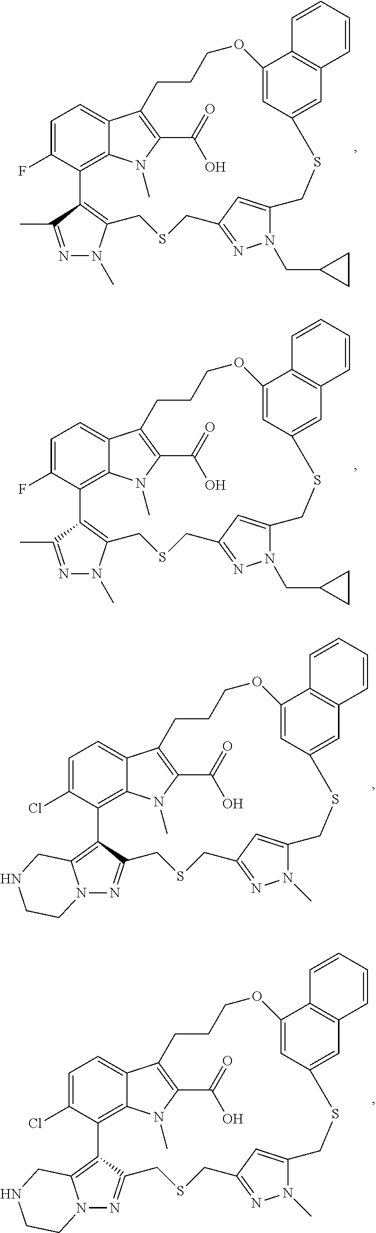

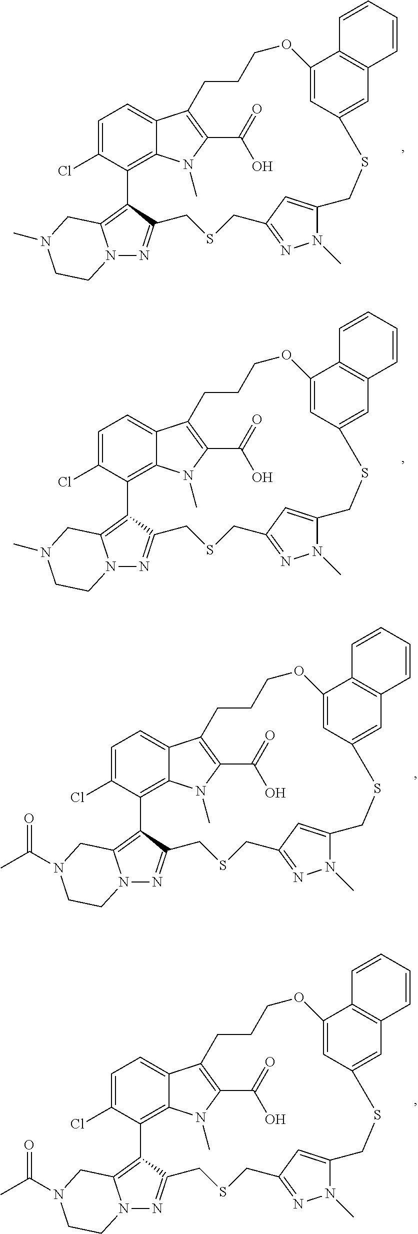

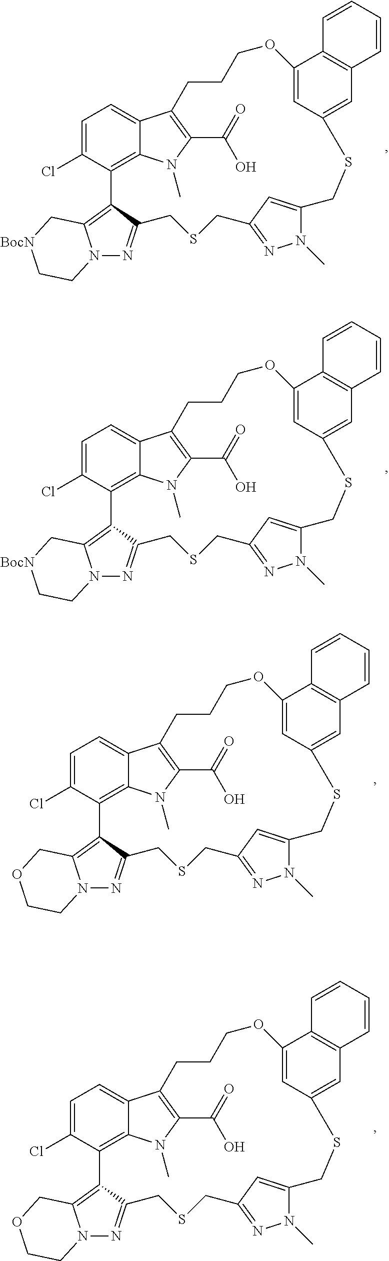

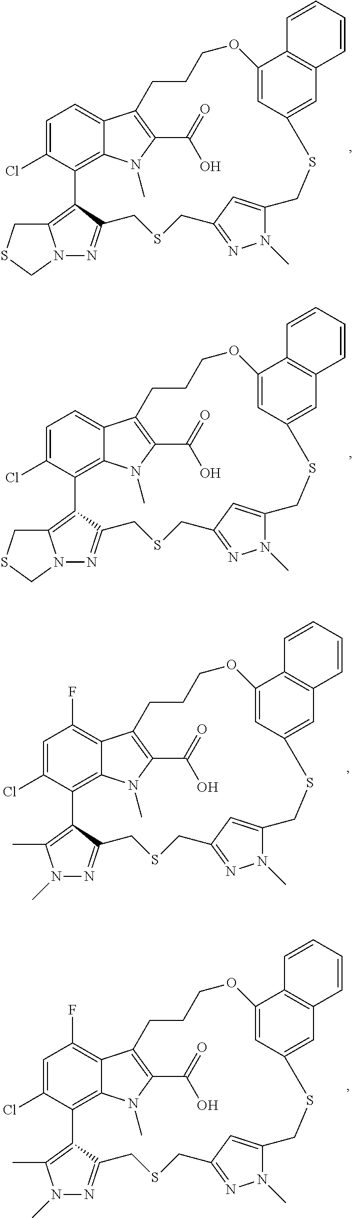

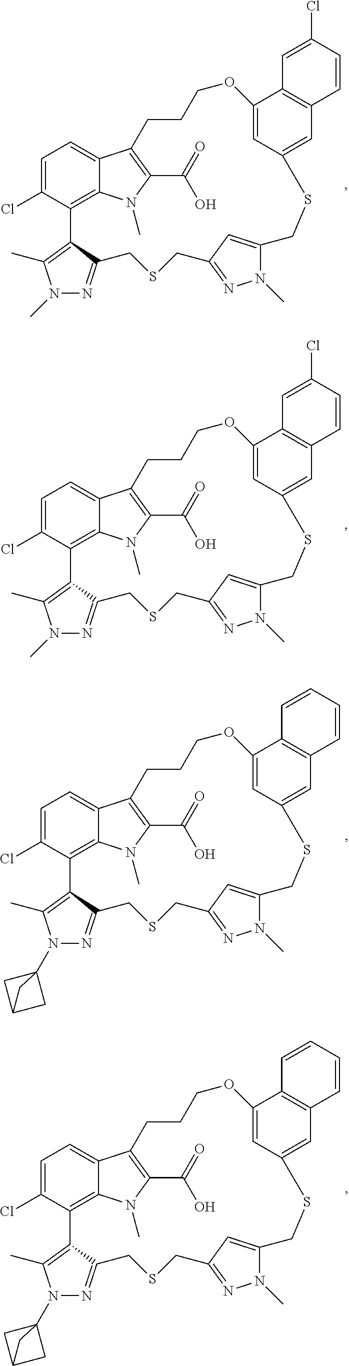

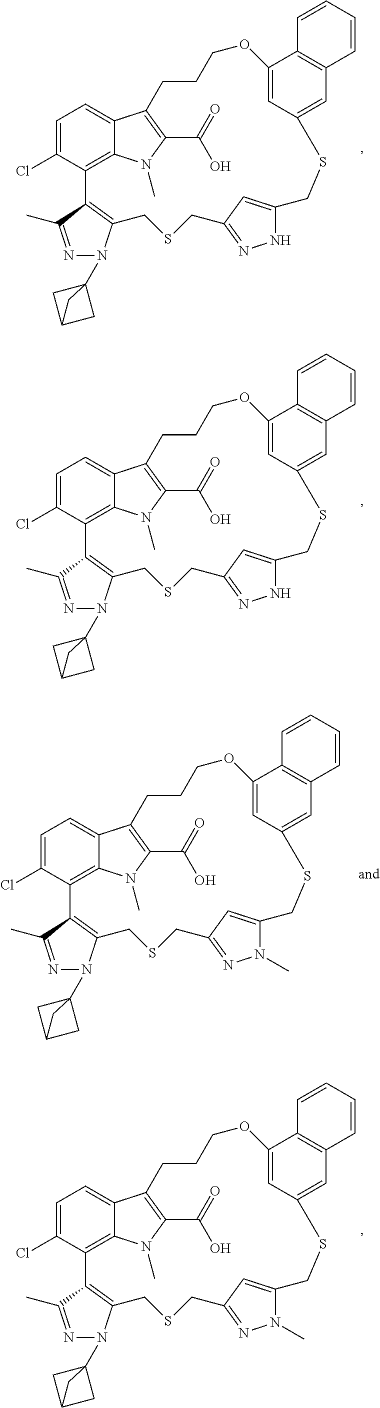

or a pharmaceutically acceptable salt of any of the foregoing.

### Synthesis

Compounds of the Formula (I), or pharmaceutically acceptable salts thereof, can be made in various ways by those skilled using known techniques as guided by the detailed teachings provided herein. For example, in an embodiment, compounds of the Formula (I) are prepared in accordance with General Scheme 1 as shown herein.

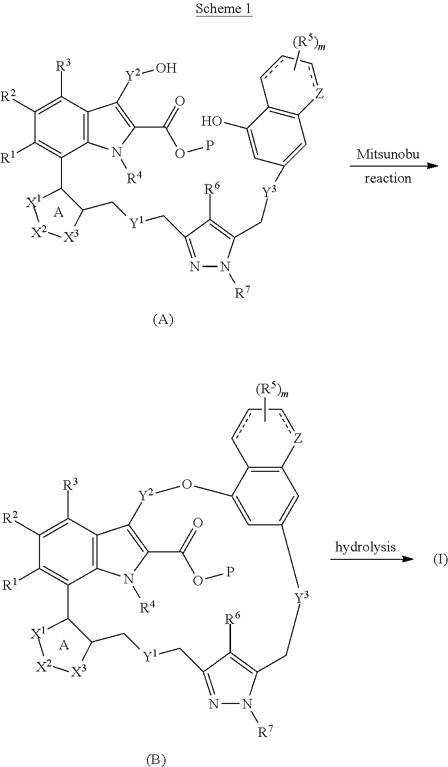

Compounds of Formula (I), and pharmaceutically acceptable salts thereof, can be prepared according to the preparation shown in Scheme 1. Compound A can undergo a Mitsunobu reaction and close the ring to form the macrocyclic Compound B. In Scheme 1, P represents a suitable protecting group. Removal of the protecting group via a hydrolysis reaction provides a compound of Formula (I), or a pharmaceutically acceptable salt thereof.

### Pharmaceutical Compositions

Some embodiments described herein relate to a pharmaceutical composition, that can include an effective amount of one or more compounds described herein (for example, a compound of Formula (I), or a pharmaceutically acceptable salt thereof) and a pharmaceutically acceptable carrier, diluent, excipient or combination thereof.

The term “pharmaceutical composition” refers to a mixture of one or more compounds and/or salts disclosed herein with other chemical components, such as diluents or carriers. The pharmaceutical composition facilitates administration of the compound to an organism. Pharmaceutical compositions can also be obtained by reacting compounds with inorganic or organic acids such as hydrochloric acid, hydrobromic acid, sulfuric acid, nitric acid, phosphoric acid, methanesulfonic acid, ethanesulfonic acid, p-toluenesulfonic acid, and salicylic acid. Pharmaceutical compositions will generally be tailored to the specific intended route of administration.

The term “physiologically acceptable” defines a carrier, diluent or excipient that does not abrogate the biological activity and properties of the compound nor cause appreciable damage or injury to an animal to which delivery of the composition is intended.

As used herein, a “carrier” refers to a compound that facilitates the incorporation of a compound into cells or tissues. For example, without limitation, dimethyl sulfoxide (DMSO) is a commonly utilized carrier that facilitates the uptake of many organic compounds into cells or tissues of a subject.

As used herein, a “diluent” refers to an ingredient in a pharmaceutical composition that lacks appreciable pharmacological activity but may be pharmaceutically necessary or desirable. For example, a diluent may be used to increase the bulk of a potent drug whose mass is too small for manufacture and/or administration. It may also be a liquid for the dissolution of a drug to be administered by injection, ingestion or inhalation. A common form of diluent in the art is a buffered aqueous solution such as, without limitation, phosphate buffered saline that mimics the pH and isotonicity of human blood.

As used herein, an “excipient” refers to an essentially inert substance that is added to a pharmaceutical composition to provide, without limitation, bulk, consistency, stability, binding ability, lubrication, disintegrating ability etc., to the composition. For example, stabilizers such as anti-oxidants and metal-chelating agents are excipients. In an embodiment, the pharmaceutical composition comprises an anti-oxidant and/or a metal-chelating agent. A “diluent” is a type of excipient.

The pharmaceutical compositions described herein can be administered to a human patient per se, or in pharmaceutical compositions where they are mixed with other active ingredients, as in combination therapy, or carriers, diluents, excipients or combinations thereof. Proper formulation is dependent upon the route of administration chosen. Techniques for formulation and administration of the compounds described herein are known to those skilled in the art.

The pharmaceutical compositions disclosed herein may be manufactured in a manner that is itself known, e.g., by means of conventional mixing, dissolving, granulating, dragee-making, levigating, emulsifying, encapsulating, entrapping or tableting processes. Additionally, the active ingredients are contained in an amount effective to achieve its intended purpose. Many of the compounds used in the pharmaceutical combinations disclosed herein may be provided as salts with pharmaceutically compatible counterions.

Multiple techniques of administering a compound, salt and/or composition exist in the art including, but not limited to, oral, rectal, pulmonary, topical, aerosol, injection, infusion and parenteral delivery, including intramuscular, subcutaneous, intravenous, intramedullary injections, intrathecal, direct intraventricular, intraperitoneal, intranasal and intraocular injections. In some embodiments, a compound of Formula (I), or a pharmaceutically acceptable salt thereof, can be administered orally.

One may also administer the compound, salt and/or composition in a local rather than systemic manner, for example, via injection or implantation of the compound directly into the affected area, often in a depot or sustained release formulation. Furthermore, one may administer the compound in a targeted drug delivery system, for example, in a liposome coated with a tissue-specific antibody. The liposomes will be targeted to and taken up selectively by the organ. For example, intranasal or pulmonary delivery to target a respiratory disease or condition may be desirable.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Compositions that can include a compound and/or salt described herein formulated in a compatible pharmaceutical carrier may also be prepared, placed in an appropriate container, and labeled for treatment of an indicated condition.

### Uses and Methods of Treatment

Some embodiments described herein relate to a method for ameliorating and/or treating a cancer described herein that can include administering an effective amount of a compound described herein (for example, a compound of Formula (I), or a pharmaceutically acceptable salt thereof) or a pharmaceutical composition that includes an effective amount of a compound described herein (for example, a compound of Formula (I), or a pharmaceutically acceptable salt thereof) to a subject having a cancer described herein. Other embodiments described herein relate to the use of an effective amount of a compound described herein (for example, a compound of Formula (I), or a pharmaceutically acceptable salt thereof) or a pharmaceutical composition that includes an effective amount of a compound described herein (for example, a compound of Formula (I), or a pharmaceutically acceptable salt thereof) in the manufacture of a medicament for ameliorating and/or treating a cancer described herein. Still other embodiments described herein relate to an effective amount of a compound described herein (for example, a compound of Formula (I), or a pharmaceutically acceptable salt thereof) or a pharmaceutical composition that includes an effective amount of a compound described herein (for example, a compound of Formula (I), or a pharmaceutically acceptable salt thereof) for ameliorating and/or treating a cancer described herein.

Some embodiments described herein relate to a method for inhibiting replication of a malignant growth or a tumor that can include contacting the growth or the tumor with an effective amount of a compound described herein (for example, a compound of Formula (I), or a pharmaceutically acceptable salt thereof) or a pharmaceutical composition that includes an effective amount of a compound described herein (for example, a compound of Formula (I), or a pharmaceutically acceptable salt thereof), wherein the malignant growth or tumor is due to a cancer described herein. Other embodiments described herein relate to the use of an effective amount of a compound described herein (for example, a compound of Formula (I), or a pharmaceutically acceptable salt thereof) or a pharmaceutical composition that includes an effective amount of a compound described herein (for example, a compound of Formula (I), or a pharmaceutically acceptable salt thereof) in the manufacture of a medicament for inhibiting replication of a malignant growth or a tumor, wherein the malignant growth or tumor is due to a cancer described herein. Still other embodiments described herein relate to an effective amount of a compound described herein (for example, a compound of Formula (I), or a pharmaceutically acceptable salt thereof) or a pharmaceutical composition that includes an effective amount of a compound described herein (for example, a compound of Formula (I), or a pharmaceutically acceptable salt thereof) for inhibiting replication of a malignant growth or a tumor, wherein the malignant growth or tumor is due to a cancer described herein.

Some embodiments described herein relate to a method for ameliorating or treating a cancer described herein that can include contacting a malignant growth or a tumor with an effective amount of a compound described herein (for example, a compound of Formula (I), or a pharmaceutically acceptable salt thereof) or a pharmaceutical composition that includes an effective amount of a compound described herein (for example, a compound of Formula (I), or a pharmaceutically acceptable salt thereof) to a subject having a cancer described herein. Other embodiments described herein relate to the use of an effective amount of a compound described herein (for example, a compound of Formula (I), or a pharmaceutically acceptable salt thereof) or a pharmaceutical composition that includes an effective amount of a compound described herein (for example, a compound of Formula (I), or a pharmaceutically acceptable salt thereof) in the manufacture of a medicament for ameliorating or treating a cancer that can include contacting a malignant growth or a tumor, wherein the malignant growth or tumor is due to a cancer described herein. Still other embodiments described herein relate to an effective amount of a compound described herein (for example, a compound of Formula (I), or a pharmaceutically acceptable salt thereof) or a pharmaceutical composition that includes an effective amount of a compound described herein (for example, a compound of Formula (I), or a pharmaceutically acceptable salt thereof) for ameliorating or treating a cancer that can include contacting a malignant growth or a tumor, wherein the malignant growth or tumor is due to a cancer described herein.

Some embodiments described herein relate to a method for inhibiting the activity of Mcl-1 that can include providing an effective amount of a compound described herein (for example, a compound of Formula (I), or a pharmaceutically acceptable salt thereof) or a pharmaceutical composition that includes an effective amount of a compound described herein (for example, a compound of Formula (I), or a pharmaceutically acceptable salt thereof) to a cancer cell from a cancer described herein. Other embodiments described herein relate to the use of an effective amount of a compound described herein (for example, a compound of Formula (I), or a pharmaceutically acceptable salt thereof) or a pharmaceutical composition that includes an effective amount of a compound described herein (for example, a compound of Formula (I), or a pharmaceutically acceptable salt thereof) in the manufacture of a medicament for inhibiting the activity of Mcl-1. Still other embodiments described herein relate to an effective amount of a compound described herein (for example, a compound of Formula (I), or a pharmaceutically acceptable salt thereof) or a pharmaceutical composition that includes an effective amount of a compound described herein (for example, a compound of Formula (I), or a pharmaceutically acceptable salt thereof) for inhibiting the activity of Mcl-1. Some embodiments described herein relate to a method for inhibiting the activity of Mcl-1 that can include providing an effective amount of a compound described herein (for example, a compound of Formula (I), or a pharmaceutically acceptable salt thereof) or a pharmaceutical composition that includes an effective amount of a compound described herein (for example, a compound of Formula (I), or a pharmaceutically acceptable salt thereof) to a cancer cell from a cancer described herein. Other embodiments described herein relate to a method for inhibiting the activity of Mcl-1 that can include contacting a cancer cell from a cancer described herein with an effective amount of a compound described herein (for example, a compound of Formula (I), or a pharmaceutically acceptable salt thereof) or a pharmaceutical composition that includes an effective amount of a compound described herein (for example, a compound of Formula (I), or a pharmaceutically acceptable salt thereof), and thereby inhibiting the activity of Mcl-1.

Some embodiments described herein relate to a method for ameliorating or treating a cancer described herein that can include inhibiting the activity of Mcl-1 using an effective amount of a compound described herein (for example, a compound of Formula (I), or a pharmaceutically acceptable salt thereof) or a pharmaceutical composition that includes an effective amount of a compound described herein (for example, a compound of Formula (I), or a pharmaceutically acceptable salt thereof). Other embodiments described herein relate to the use of an effective amount of a compound described herein (for example, a compound of Formula (I), or a pharmaceutically acceptable salt thereof) or a pharmaceutical composition that includes an effective amount of a compound described herein (for example, a compound of Formula (I), or a pharmaceutically acceptable salt thereof) in the manufacture of a medicament for ameliorating or treating a cancer described herein by inhibiting the activity of Mcl-1. Still other embodiments described herein relate to an effective amount of a compound described herein (for example, a compound of Formula (I), or a pharmaceutically acceptable salt thereof) or a pharmaceutical composition that includes an effective amount of a compound described herein (for example, a compound of Formula (I), or a pharmaceutically acceptable salt thereof) for ameliorating or treating a cancer described herein by inhibiting the activity of Mcl-1. Some embodiments described herein relate to a method for ameliorating or treating a cancer described herein that can include contacting a cancer cell with an effective amount of a compound described herein (for example, a compound of Formula (I), or a pharmaceutically acceptable salt thereof) or a pharmaceutical composition that includes an effective amount of a compound described herein (for example, a compound of Formula (I), or a pharmaceutically acceptable salt thereof), wherein the compound inhibits the activity of Mcl-1.

Some embodiments disclosed herein relate to a method for inhibiting the activity of Mcl-1 that can include providing an effective amount of a compound described herein (for example, a compound of Formula (I), or a pharmaceutically acceptable salt thereof) or a pharmaceutical composition that includes an effective amount of a compound described herein (for example, a compound of Formula (I), or a pharmaceutically acceptable salt thereof) to a subject having a cancer described herein or a cancer cell from a cancer described herein. Other embodiments disclosed herein relate to the use of an effective amount of a compound described herein (for example, a compound of Formula (I), or a pharmaceutically acceptable salt thereof) or a pharmaceutical composition that includes an effective amount of a compound described herein (for example, a compound of Formula (I), or a pharmaceutically acceptable salt thereof) in the manufacture of a medicament for inhibiting the activity of Mcl-1. Still other embodiments disclosed herein relate to a compound described herein (for example, a compound of Formula (I), or a pharmaceutically acceptable salt thereof) or a pharmaceutical composition that includes an effective amount of a compound described herein (for example, a compound of Formula (I), or a pharmaceutically acceptable salt thereof) for inhibiting the activity of Mcl-1.

Examples of suitable cancers include, but are not limited to: hematological malignancies (such as acute myeloid leukemia, multiple myeloma, mantle cell lymphoma, chronic lymphocytic leukemia, diffuse large B cell lymphoma, Burkitt's lymphoma, follicular lymphoma) and solid tumors, for example, non-small cell lung cancer (NSCLC), small cell lung cancer (SCLC), breast cancer, neuroblastoma, prostate cancer, melanoma, pancreatic cancer, uterine, endometrial, colon, oesophagus and liver cancers, osteosarcoma, Hodgkin lymphoma, mesothelioma, meningioma, glioma and tumors of upper aerodigestive, ovarian, thyroid, stomach and urinary tract.

As described herein, a cancer can become resistant to one or more anti-cancer agents. In some embodiments, a compound described herein (for example, a compound of Formula (I), or a pharmaceutically acceptable salt thereof) or a pharmaceutical composition that includes an effective amount of a compound described herein (for example, a compound of Formula (I), or a pharmaceutically acceptable salt thereof) can be used to treat and/or ameliorate a cancer that has become resistant to one or more anti-cancer agents (such as one or more Mcl-1 inhibitors). Examples of anti-cancer agents that a subject may have developed resistance to include, but are not limited to, Mcl-1 inhibitors (such as AT101, gambogic acid, TW-37, AZD5991, Sabutoclax (BI-97C1), Maritoclax, UMI-77, A-1210477, 563845, MIK665/S64315, (−)BI97D6 and/or AMG176). In some embodiments, the cancer that has become resistant to one or more anti-cancer agents can be a cancer described herein.

Several known Mcl-1 inhibitors can cause one or more undesirable side effects in the subject being treated. Examples of undesirable side effects include, but are not limited to, thrombocytopenia, neutropenia, anemia, diarrhea, vomiting, nausea, abdominal pain, and constipation. In some embodiments, a compound described herein (for example, a compound of Formula (I), or a pharmaceutically acceptable salt thereof) can decrease the number and/or severity of one or more side effects associated with a known Mcl-1 inhibitor. In some embodiments, a compound of Formula (I), or a pharmaceutically acceptable salt thereof, can result in a severity of a side effect (such as one of those described herein) that is 25% less than compared to the severity of the same side effect experienced by a subject receiving a known Mcl-1 inhibitor (such as AT101, gambogic acid, TW-37, AZD5991, Sabutoclax (BI-97C1), Maritoclax, UMI-77, A-1210477, S63845, MIK665/S64315, (−)BI97D6 and/or AMG176). In some embodiments, a compound of Formula (I), or a pharmaceutically acceptable salt thereof, results in a number of side effects that is 25% less than compared to the number of side effects experienced by a subject receiving a known Mcl-1. In some embodiments, a compound of Formula (I), or a pharmaceutically acceptable salt thereof, results in a severity of a side effect (such as one of those described herein) that is less in the range of about 10% to about 30% compared to the severity of the same side effect experienced by a subject receiving a known Mcl-1. In some embodiments, a compound of Formula (I), or a pharmaceutically acceptable salt thereof, results in a number of side effects that is in the range of about 10% to about 30% less than compared to the number of side effects experienced by a subject receiving a known Mcl-1.

The one or more compounds of Formula (I), or a pharmaceutically acceptable salt thereof, that can be used to treat, ameliorate and/or inhibit the growth of a cancer wherein inhibiting the activity of Mcl-1 is beneficial is provided in any of the embodiments described in paragraphs [0064]-[0084], under the heading titled “Compounds.”

As used herein, a “subject” refers to an animal that is the object of treatment, observation or experiment. “Animal” includes cold- and warm-blooded vertebrates and invertebrates such as fish, shellfish, reptiles and, in particular, mammals. “Mammal” includes, without limitation, mice, rats, rabbits, guinea pigs, dogs, cats, sheep, goats, cows, horses, primates, such as monkeys, chimpanzees, and apes, and, in particular, humans. In some embodiments, the subject can be human. In some embodiments, the subject can be a child and/or an infant, for example, a child or infant with a fever. In other embodiments, the subject can be an adult.

As used herein, the terms “treat,” “treating,” “treatment,” “therapeutic,” and “therapy” do not necessarily mean total cure or abolition of the disease or condition. Any alleviation of any undesired signs or symptoms of the disease or condition, to any extent can be considered treatment and/or therapy. Furthermore, treatment may include acts that may worsen the subject's overall feeling of well-being or appearance.

The terms “therapeutically effective amount” and “effective amount” are used to indicate an amount of an active compound, or pharmaceutical agent, that elicits the biological or medicinal response indicated. For example, a therapeutically effective amount of compound, salt or composition can be the amount needed to prevent, alleviate or ameliorate symptoms of the disease or condition, or prolong the survival of the subject being treated. This response may occur in a tissue, system, animal or human and includes alleviation of the signs or symptoms of the disease or condition being treated. Determination of an effective amount is well within the capability of those skilled in the art, in view of the disclosure provided herein. The therapeutically effective amount of the compounds disclosed herein required as a dose will depend on the route of administration, the type of animal, including human, being treated and the physical characteristics of the specific animal under consideration. The dose can be tailored to achieve a desired effect, but will depend on such factors as weight, diet, concurrent medication and other factors which those skilled in the medical arts will recognize.

For example, an effective amount of a compound, or radiation, is the amount that results in: (a) the reduction, alleviation or disappearance of one or more symptoms caused by the cancer, (b) the reduction of tumor size, (c) the elimination of the tumor, and/or (d) long-term disease stabilization (growth arrest) of the tumor. In the treatment of lung cancer (such as non-small cell lung cancer) a therapeutically effective amount is that amount that alleviates or eliminates cough, shortness of breath and/or pain. As another example, an effective amount, or a therapeutically effective amount of a Mcl-1 inhibitor is the amount which results in the reduction in Mcl-1 activity and/or phosphorylation (such as phosphorylation of CDCl₂). The reduction in Mcl-1 activity is known to those skilled in the art and can be determined by the analysis of Mcl-1 intrinsic kinase activity and downstream substrate phosphorylation.

The amount of the compound of Formula (I), or a pharmaceutically acceptable salt thereof, required for use in treatment will vary not only with the particular compound or salt selected but also with the route of administration, the nature and/or symptoms of the disease or condition being treated and the age and condition of the patient and will be ultimately at the discretion of the attendant physician or clinician. In cases of administration of a pharmaceutically acceptable salt, dosages may be calculated as the free base. As will be understood by those of skill in the art, in certain situations it may be necessary to administer the compounds disclosed herein in amounts that exceed, or even far exceed, the dosage ranges described herein in order to effectively and aggressively treat particularly aggressive diseases or conditions.

In general, however, a suitable dose will often be in the range of from about 0.05 mg/kg to about 10 mg/kg. For example, a suitable dose may be in the range from about 0.10 mg/kg to about 7.5 mg/kg of body weight per day, such as about 0.15 mg/kg to about 5.0 mg/kg of body weight of the recipient per day, about 0.2 mg/kg to 4.0 mg/kg of body weight of the recipient per day, or any amount in between. The compound may be administered in unit dosage form; for example, containing 1 to 500 mg, 10 to 100 mg, 5 to 50 mg or any amount in between, of active ingredient per unit dosage form.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations.

As will be readily apparent to one skilled in the art, the useful in vivo dosage to be administered and the particular mode of administration will vary depending upon the age, weight, the severity of the affliction, the mammalian species treated, the particular compounds employed and the specific use for which these compounds are employed. The determination of effective dosage levels, that is the dosage levels necessary to achieve the desired result, can be accomplished by one skilled in the art using routine methods, for example, human clinical trials, in vivo studies and in vitro studies. For example, useful dosages of a compound of Formula (I), or pharmaceutically acceptable salts thereof, can be determined by comparing their in vitro activity, and in vivo activity in animal models. Such comparison can be done by comparison against an established drug, such as cisplatin and/or gemcitabine)

Dosage amount and interval may be adjusted individually to provide plasma levels of the active moiety which are sufficient to maintain the modulating effects, or minimal effective concentration (MEC). The MEC will vary for each compound but can be estimated from in vivo and/or in vitro data. Dosages necessary to achieve the MEC will depend on individual characteristics and route of administration. However, HPLC assays or bioassays can be used to determine plasma concentrations. Dosage intervals can also be determined using MEC value. Compositions should be administered using a regimen which maintains plasma levels above the MEC for 10-90% of the time, preferably between 30-90% and most preferably between 50-90%. In cases of local administration or selective uptake, the effective local concentration of the drug may not be related to plasma concentration.

It should be noted that the attending physician would know how to and when to terminate, interrupt or adjust administration due to toxicity or organ dysfunctions. Conversely, the attending physician would also know to adjust treatment to higher levels if the clinical response were not adequate (precluding toxicity). The magnitude of an administrated dose in the management of the disorder of interest will vary with the severity of the disease or condition to be treated and to the route of administration. The severity of the disease or condition may, for example, be evaluated, in part, by standard prognostic evaluation methods. Further, the dose and perhaps dose frequency, will also vary according to the age, body weight and response of the individual patient. A program comparable to that discussed above may be used in veterinary medicine.

Compounds, salts and compositions disclosed herein can be evaluated for efficacy and toxicity using known methods. For example, the toxicology of a particular compound, or of a subset of the compounds, sharing certain chemical moieties, may be established by determining in vitro toxicity towards a cell line, such as a mammalian, and preferably human, cell line. The results of such studies are often predictive of toxicity in animals, such as mammals, or more specifically, humans. Alternatively, the toxicity of particular compounds in an animal model, such as mice, rats, rabbits, dogs or monkeys, may be determined using known methods. The efficacy of a particular compound may be established using several recognized methods, such as in vitro methods, animal models, or human clinical trials. When selecting a model to determine efficacy, the skilled artisan can be guided by the state of the art to choose an appropriate model, dose, route of administration and/or regime.

### EXAMPLES

Additional embodiments are disclosed in further detail in the following examples, which are not in any way intended to limit the scope of the claims.

### Intermediate 1

### 3-(((4-Methoxybenzyl)oxy)methyl)-1,5-dimethyl-4-(4,4,5,5-tetramethyl-1,3,2-dioxaborolan-2-yl)-1H-pyrazol (1)

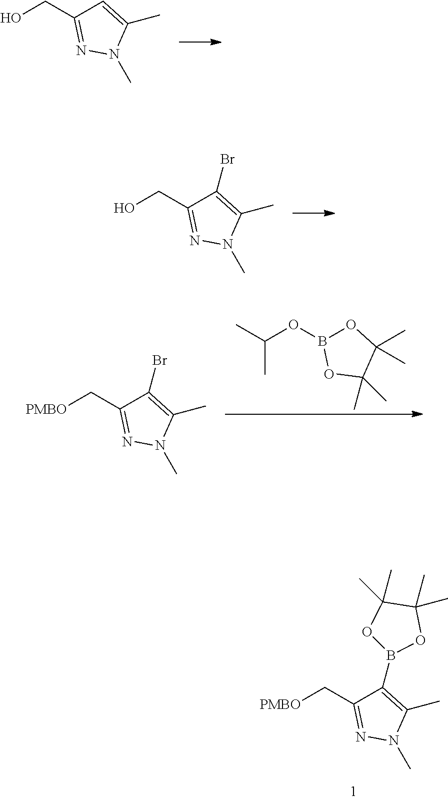

### (4-Bromo-1,5-dimethyl-1H-pyrazol-3-yl)methanol

To a stirred solution of (1,5-dimethyl-1H-pyrazol-3-yl)methanol (7 g, 55.48 mmol) in DCM (110 mL) was added NBS (10.37 g, 58.25 mmol) portion-wise over 30 min at 0° C. and stirred at room temperature (rt) for 1 h. The reaction was quenched with water (60 mL). The mixture was diluted with DCM (50 mL), and the layers were separated. The organic layer was washed with brine (60 mL). The organic layer was separated, dried over Na₂SO₄, filtered and evaporated to afford the semi pure compound. This was triturated with 20% EtOAc in petroleum ether (PE) to afford (4-bromo-1,5-dimethyl-1H-pyrazol-3-yl)methanol (8 g, 39.01 mmol, 70%) as a light yellow solid. MS (LCMS) m/z 205.0 [M+H]⁺.

### 4-Bromo-3-(((4-methoxybenzyl)oxy)methyl)-1,5-dimethyl-1H-pyrazole

To a stirred solution of (4-bromo-1,5-dimethyl-1H-pyrazol-3-yl)methanol (2×4 g, 19.51 mmol) in DMF (40 mL) was added NaH (60% in oil, 538 mg, 22.39 mmol) at 0° C. The mixture was stirred at rt for 20 min. 1-(Chloromethyl)-4-methoxybenzene (3.36 g, 21.46 mmol) and KI (324 mg, 1.95 mmol) were added, and the mixture was stirred at rt for 16 h. The reaction was quenched with sat. aq. NH₄Cl solution (25 mL). The mixture was extracted with EtOAc (3×40 mL). The combined organic layer was washed with water (2×25 mL) and brine (25 mL), dried over Na₂SO₄ and concentrated under reduced pressure to afford the semi pure compound. The compound was purified by silica gel column chromatography eluting with 10-20% EtOAc:PE to afford 4-bromo-3-(((4-methoxybenzyl)oxy)methyl)-1,5-dimethyl-1H-pyrazole (8 g, 24.60 mmol, 63% for two batches) as an off white solid. MS (LCMS) m/z 325.0 [M+H]⁺.

### 3-(((4-Methoxybenzyl)oxy)methyl)-1,5-dimethyl-4-(4,4,5,5-tetramethyl-1,3,2-dioxaborolan-2-yl)-1H-pyrazole (1)

To a stirred solution of 4-bromo-3-(((4-methoxybenzyl)oxy)methyl)-1,5-dimethyl-1H-pyrazole (2×4 g, 12.30 mmol) in THF (110 mL) was added n-BuLi (1.6 M in hexanes, 15.4 mL, 24.6 mmol) at −78° C., and the mixture was stirred at −78° C. for 50 min. 2-isopropoxy-4,4,5,5-tetramethyl-1,3,2-dioxaborolane (3.2 g, 17.22 mmol) was added at −78° C. The mixture temperature was slowly raised to rt and stirred for 4 h. The solvents were evaporated. The mixture was diluted with EtOAc (75 mL), filtered through a Celite pad, and filtrate was evaporated to afford semi pure compound. The compound was purified by silica gel column chromatography eluting with 30% EtOAc/PE to afford 3-(((4-methoxybenzyl)oxy)methyl)-1,5-dimethyl-4-(4,4,5,5-tetramethyl-1,3,2-dioxaborolan-2-yl)-1H-pyrazole (1) (5 g, 13.43 mmol, 54% for two batches) as a colourless liquid. MS (LCMS) m/z 373.17 [M+H]⁺.

### Intermediate 2

### Methyl 3-(3-acetoxypropyl)-7-bromo-6-chloro-1-methyl-1H-indole-2-carboxylate (2)

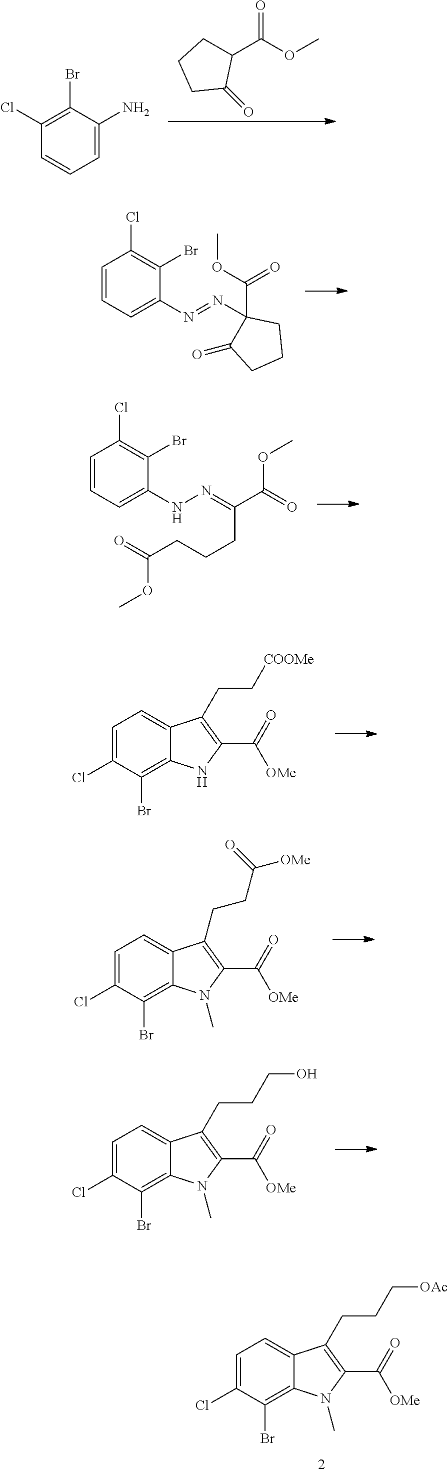

### Methyl 1-((2-bromo-3-chlorophenyl)diazenyl)-2-oxocyclopentane-1-carboxylate

To a stirred solution of 2-bromo-3-chloroaniline (25 g, 121.3 mmol) in conc. HCl (62.5 mL) was added water (62.5 mL), and the mixture was stirred at rt for 16 h. Then a solution of NaNO₂ (8.79 g, 127.4 mmol) in water (30 mL) was added at 0° C., and the mixture stirred at rt for 1.5 h. A solution of KOAc (166.5 g, 1699 mmol) in water (250 mL) and methyl 2-oxocyclopentane-1-carboxylate was added dropwise. The mixture stirred at 0-5° C. for 0.5 h and then at rt for 2 h. The solution was extracted with DCM (3×400 mL). The combined organic layer was washed with brine (200 mL), dried over Na₂SO₄, filtered and evaporated to afford methyl 1-((2-bromo-3-chlorophenyl)diazenyl)-2-oxocyclopentane-1-carboxylate (42 g, 116.8 mmol, 96%) as a red solid. This compound was used without further purification in the next step. MS (LCMS) m/z 359.0 [M+H]⁺.

### Dimethyl (E/Z)-2-(2-(2-bromo-3-chlorophenyl)hydrazineylidene)hexanedioate

To a stirred solution of methyl 1-((2-bromo-3-chlorophenyl)diazenyl)-2-oxocyclopentane-1-carboxylate (42 g, 117.3 mmol) in MeOH (420 mL) was added conc. H₂SO₄ (30 mL, 566.6 mmol) at 0° C. The mixture was stirred at 80° C. for 2 h, and then cooled to rt. The precipitated solids were filtered and washed with MeOH to afford dimethyl (E/Z)-2-(2-(2-bromo-3-chlorophenyl)hydrazineylidene)hexanedioate (28 g, 71.49 mmol, 61%) as a pale yellow solid. This compound was used without further purification in the next step. MS (LCMS) m/z 391.18 [M+H]⁺.

### Methyl 7-bromo-6-chloro-3-(3-methoxy-3-oxopropyl)-1H-indole-2-carboxylate

To a stirred solution of dimethyl (E/Z)-2-(2-(2-bromo-3 chlorophenyl)hydrazineylidene)hexanedioate (29 g, 74.05 mmol) in MeOH (290 mL) was added conc. H₂SO₄ (50 mL, 938 mmol) at 0° C. The mixture was stirred at 80° C. for 4 days, and then cooled to rt. The precipitated solids were filtered, washed with MeOH and dried to afford methyl 7-bromo-6-chloro-3-(3-methoxy-3-oxopropyl)-1H-indole-2-carboxylate (14 g, 37.37 mmol, 50%) as an off white solid. MS (LCMS) m/z 373.93 [M+H]⁺.

### Methyl 7-bromo-6-chloro-3-(3-methoxy-3-oxopropyl)-1-methyl-1H-indole-2-carboxylate

To a stirred solution of methyl 7-bromo-6-chloro-3-(3-methoxy-3-oxopropyl)-1H-indole-2-carboxylate (20 g, 53.619 mmol) in dry DMF (200 mL) were added Cs₂CO₃ (26.2 g, 80.43 mmol) and Mel (6.68 mL, 107.2 mmol). The mixture stirred at rt for 3 h. The reaction was quenched with water (500 mL) and extracted with EtOAc (3×200 mL). The organic layer was separated, dried over Na₂SO₄, filtered and evaporated to afford semi-pure methyl 7-bromo-6-chloro-3-(3-methoxy-3-oxopropyl)-1-methyl-1H-indole-2-carboxylate (18 g, 46.31 mmol, 86%) as a brown liquid. This compound was used without further purification in the next step. MS (LCMS) m/z 388.12 [M+H]⁺.

### Methyl 7-bromo-6-chloro-3-(3-hydroxypropyl)-1-methyl-1H-indole-2-carboxylate

To a suspension of methyl 7-bromo-6-chloro-3-(3-methoxy-3-oxopropyl)-1-methyl-1H-indole-2-carboxylate (18 g, 46.511 mmol) in dry THF (180 mL) was added BH₃.THF (1.0 M in THF, 255.8 mL, 255.8 mmol) dropwise at 0° C. The temperature was raised to rt, and the mixture stirred for 6 h. The reaction was quenched with MeOH (255 mL) and 6N HCl (255 mL) at 0° C. The mixture was stirred for 10 min and then at rt for 20 min. The mixture was further diluted with water (500 mL) and extracted with 10% MeOH in DCM (3×500 mL). The organic layer was separated, dried over Na₂SO₄, filtered and evaporated to afford the semi-pure compound. The compound was purified by silica gel column chromatography using 25% EtOAc in PE to afford methyl 7-bromo-6-chloro-3-(3-hydroxypropyl)-1-methyl-1H-indole-2-carboxylate (14 g, 3.899 mmol, 72%) as an off white solid. MS (LCMS) m/z 360.11 [M+H]⁺.

### Methyl 3-(3-acetoxypropyl)-7-bromo-6-chloro-1-methyl-1H-indole-2-carboxylate

To a stirred solution of methyl 7-bromo-6-chloro-3-(3-hydroxypropyl)-1-methyl-1H-indole-2-carboxylate (8 g, 22.284 mmol) in DCM (80 mL) were added Et₃N (6.2 mL, 44.568 mmol), DMAP (cat.), and Ac₂O (2.53 mL, 26.74 mmol, 1.2 eq.) at 0° C. The mixture was stirred at rt for 2 h. The mixture was diluted with DCM (500 mL), washed with water (2×200 mL) and brine (200 mL). The organic layer was separated, dried over Na₂SO₄, filtered and evaporated to afford the semi-pure compound. The compound was purified by silica gel column chromatography using 10% EtOAc/PE to afford methyl 3-(3-acetoxypropyl)-7-bromo-6-chloro-1-methyl-1H-indole-2-carboxylate (2) (8 g, 19.95 mmol, 89%) as an off white solid. MS (LCMS) m/z 402.00 [M+H]⁺.

### Intermediate 3

### S-((5-(((tert-Butyldiphenylsilyl)oxy)methyl)-1-methyl-1H-pyrazol-3-yl)methyl) ethanethioate (3)

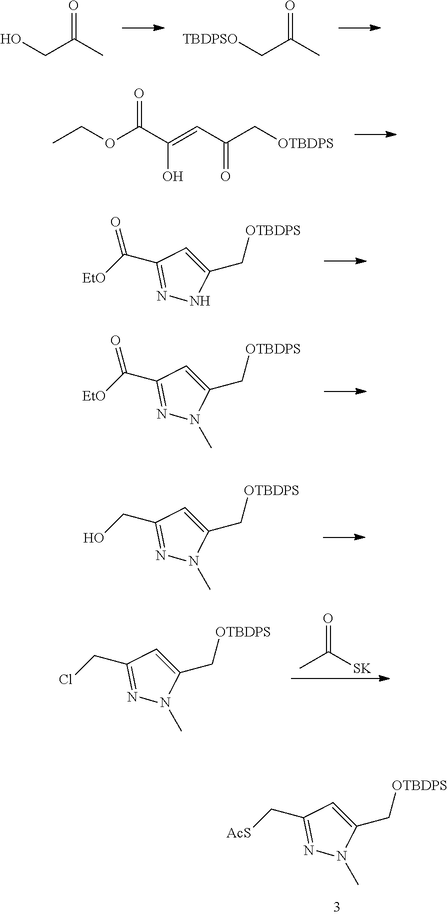

### 1-((tert-Butyldiphenylsilyl)oxy)propan-2-one

To a stirred solution of 1-hydroxypropan-2-one (50 g, 675.67 mmol) in DMF (500 mL) was added imidazole (50.54 g, 743.23 mmol) and DMAP (4.1 g, 33.78 mmol) followed by TBDPSCl (140 mL, 540.54 mmol) at 0° C. The mixture stirred at rt for 16 h. The mixture was poured in to ice water and then extracted with PE (2×400 mL). The combined organic layer was washed with brine (200 mL), dried over Na₂SO₄, filtered and evaporated to afford semi-pure 1-((tert-butyldiphenylsilyl)oxy)propan-2-one (105 g, 336.53 mmol) as a red coloured liquid. This compound was used without further purification in the next step. ¹H NMR (400 MHz, CDCl₃) δ 7.73-7.63 (m, 4H), 7.47-7.35 (m, 6H), 4.16 (s, 2H), 2.19 (s, 3H), 1.07 (s, 9H).

### Ethyl (Z)-5-((tert-butyldiphenylsilyl)oxy)-2-hydroxy-4-oxopent-2-enoate

To a stirred solution of diethyl oxalate (26.23 mL, 192.30 mmol) in THF (500 mL) was added potassium tert-butoxide (21.53 g, 192.30 mmol) at 0° C., and the mixture stirred at 0° C. for 1 h. 1-((tert-butyldiphenylsilyl)oxy)propan-2-one (50 g, 160.25 mmol) was added and stirring was continued at 0° C. for 1 h. After completion of the reaction, the mixture was diluted with EtOAc, and the reaction was quenched with 2N HCl (400 mL). The mixture was extracted with EtOAc (2×300 mL). The combined organic layer was washed with brine (200 mL), dried over Na₂SO₄, filtered and evaporated to afford semi-pure ethyl (Z)-5-((tert-butyldiphenylsilyl)oxy)-2-hydroxy-4-oxopent-2-enoate (75 g, 182.03 mmol) as a red solid. This compound was used without further purification. ¹H NMR (400 MHz, CDCl₃) δ 7.72-7.60 (m, 4H), 7.48-7.35 (m, 6H), 6.87 (s, 1H), 4.39-4.34 (m, 2H), 4.24 (s, 2H), 1.40-1.32 (m, 3H), 1.10 (s, 9H).

### Ethyl 5-(((tert-butyldiphenylsilyl)oxy)methyl)-1H-pyrazole-3-carboxylate

To a stirred solution of ethyl (Z)-5-((tert-butyldiphenylsilyl)oxy)-2-hydroxy-4-oxopent-2-enoate (150 g, 364.67 mmol) in EtOH (400 mL) was added N₂H₂.H₂O (17 mL, 364.67 mmol), and the mixture stirred at 80° C. for 2 h. The mixture was concentrated, diluted with EtOAc (300 mL) and washed with water (200 mL) and brine (150 mL). The organic layer was separated, dried over Na₂SO₄, filtered and evaporated to afford a semi-pure compound. The compound was purified by silica gel column chromatography using 15% EtOAc in PE to afford ethyl 5-(((tert-butyldiphenylsilyl)oxy)methyl)-1H-pyrazole-3-carboxylate (60 g, 146.85 mmol, 20% over three steps) as a white solid. ¹H NMR (400 MHz, CDCl₃) δ 7.72-7.62 (m, 4H), 7.45-7.35 (m, 7H), 6.67 (s, 1H), 4.79 (s, 2H), 4.41-4.33 (m, 2H), 4.24 (s, 2H), 1.41-1.36 (m, 3H), 1.08 (s, 9H).

### Ethyl 5-(((tert-butyldiphenylsilyl)oxy)methyl)-1-methyl-1H-pyrazole-3-carboxylate

To a stirred solution of ethyl 5-(((tert-butyldiphenylsilyl)oxy)methyl)-1H-pyrazole-3-carboxylate (60 g, 147.42 mmol) in THF (600 mL) was added NaHMDS (176.9 mL, 176.90 mmol) at 0° C., and the mixture was stirred at rt for 30 min. Methyl iodide (13.7 mL, 221.11 mmol) was added at 0° C. The mixture was slowly allowed to warm to rt and then the mixture was stirred for 2 h. The mixture was concentrated and diluted with EtOAc (200 mL) washed with water (200 mL) and brine (150 mL). The organic layer was separated, dried over Na₂SO₄, filtered and evaporated to afford semi-pure ethyl 5-(((tert-butyldiphenylsilyl)oxy)methyl)-1-methyl-1H-pyrazole-3-carboxylate (65 g, 153.66 mmol) as a white solid. This compound was used without further purification. MS (LCMS) m/z 423.31 [M+H]⁺.

### (5-(((tert-Butyldiphenylsilyl)oxy)methyl)-1-methyl-1H-pyrazol-3-yl)methanol

To a stirred solution of ethyl 5-(((tert-butyldiphenylsilyl)oxy)methyl)-1-methyl-1H-pyrazole-3-carboxylate (65 g, 153.66 mmol) in THF (320 mL) was added LiAlH₄ (2.4 M in THF, 8.5 mL, 19.97 mmol) at 0° C., and the mixture was stirred at 0° C. for 1 h. After completion of the reaction, the mixture was cooled to 0° C., and the reaction was quenched with a sat. NH₄Cl solution. The mixture was filtered through a Celite pad and extracted with EtOAc (4×200 mL). The organic layer was dried over Na₂SO₄, filtered and evaporated to afford a semi-pure compound. The compound was purified by silica gel column chromatography using 50% EtOAc in PE to afford (5-(((tert-butyldiphenylsilyl)oxy)methyl)-1-methyl-1H-pyrazol-3-yl)methanol (16 g, 42.04 mmol, 29% over two steps) as a pale yellow liquid. MS (LCMS) m/z 381.28 [M+H]⁺.

### 5-(((tert-Butyldiphenylsilyl)oxy)methyl)-3-(chloromethyl)-1-methyl-1H-pyrazole

To a stirred solution of (5-(((tert-butyldiphenylsilyl)oxy)methyl)-1-methyl-1H-pyrazol-3-yl)methanol (16 g, 42.10 mmol) in DCM (70 mL) under Ar was added SOCl₂ (3.66 mL, 50.52 mmol) at 0° C. The mixture was stirred at rt for 1 h. The mixture was diluted with DCM (200 mL) and washed with a sat. NaHCO₃ solution (2×100 mL). The organic layer was separated, dried over Na₂SO₄, filtered and evaporated to afford semi-pure 5-(((tert-butyldiphenylsilyl)oxy)methyl)-3-(chloromethyl)-1-methyl-1H-pyrazole (17 g, 42.6 mmol) as a red colour liquid. This compound was used without further purification. MS (LCMS) m/z 399.32 [M+H]⁺.

### S-((5-(((tert-Butyldiphenylsilyl)oxy)methyl)-1-methyl-1H-pyrazol-3-yl)methyl) ethanethioate (3)

To a stirred solution of semi-pure 5-(((tert-butyldiphenylsilyl)oxy)methyl)-3-(chloromethyl)-1-methyl-1H-pyrazole (27.7 g, 69.59 mmol) in MeCN (280 mL) under Ar was added NaI (10.43 g, 69.59 mmol) and potassium thio acetate (15.8 g, 139.19 mmol) at 0° C., and the mixture was stirred at rt for 16 h. The mixture was filter through a Celite pad washed with DCM, filtered and evaporated to afford a semi-pure compound. The compound was purified by silica gel column chromatography using 20% EtOAc in PE to afford S-((5-(((tert-butyldiphenylsilyl)oxy)methyl)-1-methyl-1H-pyrazol-3-yl)methyl) ethanethioate (3) (9.6 g, 21.88 mmol, 52% over two steps) as a red colour liquid. ¹H NMR (400 MHz, CDCl₃) δ 7.66-7.62 (m, 4H), 7.47-7.37 (m, 6H), 5.92 (s, 1H), 4.60 (s, 2H), 4.08 (s, 2H), 3.80 (s, 3H), 2.35 (s, 3H), 1.04 (s, 9H).

### Intermediate 4

### 3-(Acetylthio)naphthalen-1-yl acetate (4)

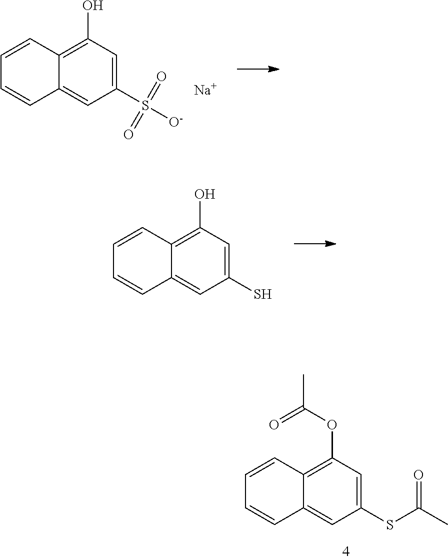 [SMILES: O=S(=O)([O-])c1cc(O)c2ccccc2c1 ; [Na+] ; Oc1cc(S)cc2ccccc12 ; CC(=O)Oc1cc(SC(C)=O)cc2ccccc12]

### 3-Mercaptonaphthalen-1-ol

To a stirred solution of sodium 4-hydroxynaphthalene-2-sulfonate (2×7 g, 28.43 mmol), TPP (29.83 g, 113.72 mmol) and 18-crown-6 (2.25 g, 8.53 mmol) in toluene (70 mL) was added I₂ (3.61 g, 14.22 mmol) at rt, and the mixture was stirred at 100° C. for 17 h. 1,4-dioxane (20 mL) and water (10 mL) were added, and the mixture was stirred at 100° C. for 1 h. Na₂SO₄ was added, and the solids were filtered and evaporated partially to afford 3-mercaptonaphthalen-1-ol (40 g in toluene) as a yellow semi solid. This compound was used without further purification.

### 3-(Acetylthio)naphthalen-1-yl acetate (4)

To a stirred solution of 3-mercaptonaphthalen-1-ol (40 g in toluene, 56.86 mmol) in DCM (400 mL) were added Et₃N (15.85 mL, 113.72 mmol), DMAP (695 mg, 5.69 mmol), and Ac₂O (32.3 mL, 341.2 mmol) at 0° C., and the mixture was stirred at rt for 3 h. The mixture was diluted with DCM (100 mL) and washed with water (3×300 mL) and brine (250 mL). The organic layer was separated, dried over Na₂SO₄, filtered and evaporated to afford the semi-pure compound. The compound was purified by silica gel column chromatography using 1-10% EtOAc:PE to afford 3-(acetylthio)naphthalen-1-yl acetate (4) (7 g, 26.89 mmol, 47% for two steps) as a light brown solid. MS (LCMS) m/z 261.01 [M+H]⁺. ¹H NMR (400 MHz, CDCl₃) δ 7.89-7.82 (m, 3H), 7.59-7.52 (m, 2H), 7.31 (d, J=1.6 Hz, 1H), 2.46 (s, 3H), 2.45 (s, 3H).

### Intermediate 5

### 1-(4-Methoxybenzyl)-3-(((4-methoxybenzyl)oxy)methyl)-5-methyl-4-(4,4,5,5-tetramethyl-1,3,2-dioxaborolan-2-yl)-1H-pyrazole (5)

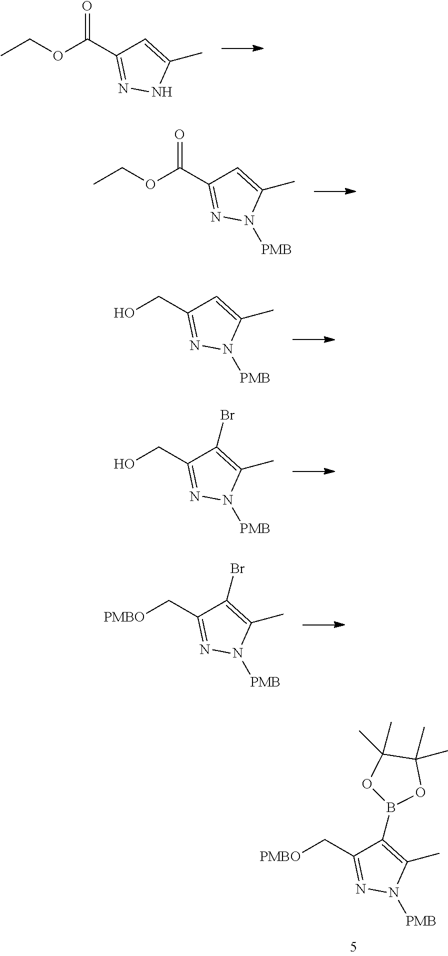

### Ethyl 1-(4-methoxybenzyl)-5-methyl-1H-pyrazole-3-carboxylate

To a stirred solution of ethyl 5-methyl-1H-pyrazole-3-carboxylate (20 g, 129.72 mmol) in THF (300 mL) were added 2M NaHMDS (77.83 mL, 155.66 mmol), PMB-Cl (21 mL, 155.66 mmol) and NaI (9.721 g, 64.86 mmol) at 0° C., and the mixture was heated at 50° C. for 2 h. After consumption of starting material, the mixture was cooled to −10° C. The reaction was quenched with sat. aq. NH₄Cl solution and extracted with EtOAc (2×200 mL). The combined organic layer was dried over anhydrous Na₂SO₄, filtered and concentrated under reduced pressure to afford ethyl 1-(4-methoxybenzyl)-5-methyl-1H-pyrazole-3-carboxylate (40 g, 145.98 mmol, LCMS: 83%) of as a light yellow gummy liquid. The material was used to next step as such without purification. MS (ESI) m/z 275.21 [M+1]⁺. ¹H NMR (400 MHz, CDCl₃) δ 7.08-7.05 (m, 2H), 6.85-6.80 (m, 2H), 6.59 (s, 1H), 4.12 (q, J=7.2 Hz, 2H), 3.81-3.75 (m, 5H), 2.18 (s, 3H), 1.40 (t, J=7.2 Hz, 3H).

### (1-(4-methoxybenzyl)-5-methyl-1H-pyrazol-3-yl)methanol

To a stirred solution of ethyl 1-(4-methoxybenzyl)-5-methyl-1H-pyrazole-3-carboxylate (40 g, 145.98 mmol) in THF (400 mL) was added LiAlH₄ (2.4 M in THF, 66.9 mL, 160.57 mmol) at 0° C., and the mixture was stirred at rt for 3 h. After consumption of starting material, the reaction was quenched with ice cooled sat. Na₂SO₄ solution at 0° C. The resulting slurry was filtered on a Celite bed. The Celite bed was washed with ethyl acetate (2000 mL). The filtrate was dried over Na₂SO₄, filtered and evaporated to give a semi pure (1-(4-methoxybenzyl)-5-methyl-1H-pyrazol-3-yl)methanol (36 g) as a pale yellow gummy liquid. The material was used to next step as such without purification. MS (ESI) m/z 233.16 [M+1]⁺. ¹H NMR (400 MHz, CDCl₃) δ 7.08-7.03 (m, 2H), 6.85-6.81 (m, 2H), 6.04 (s, 1H), 5.18 (s, 2H), 4.65 (s, 2H), 3.81-3.75 (m, 4H), 2.19 (s, 3H).

### (4-bromo-1-(4-methoxybenzyl)-5-methyl-1H-pyrazol-3-yl)methanol

To a stirred solution of (1-(4-methoxybenzyl)-5-methyl-1H-pyrazol-3-yl)methanol (36 g, 155.17 mmol) in DCM (540 mL) was added NBS (30.38 g, 170.68 mmol) portionwise over 30 min at 0° C., and the mixture was stirred at rt for 3 h. After completion of reaction, the mixture was diluted with DCM (500 mL) and washed with water (300 mL). The layers were separated and washed with brine (250 mL). The organic layer was dried over anhydrous Na₂SO₄, filtered and evaporated under reduced pressure. The obtained crude compound was purified by silica gel column and eluted at 40% EtOAc in petroleum ether (PE) to afford (4-bromo-1-(4-methoxybenzyl)-5-methyl-1H-pyrazol-3-yl)methanol (24.5 g, 79.03 mmol, 60% over 3 steps) as a pale yellow gummy liquid. MS (ESI) m/z 313.12 [M+2]⁺. ¹H NMR (400 MHz, CDCl₃) δ 7.07 (d, J=8.8 Hz, 2H), 6.85 (d, J=8.8 Hz, 2H), 5.21 (s, 2H), 4.67 (s, 2H), 3.79 (s, 3H), 2.18 (s, 3H).

### 4-bromo-1-(4-methoxybenzyl)-3-(((4-methoxybenzyl)oxy)methyl)-5-methyl-1H-pyrazole

To a stirred solution of (4-bromo-1-(4-methoxybenzyl)-5-methyl-1H-pyrazol-3-yl)methanol (24.5 g, 79.03 mmol) in DMF (250 mL) was added NaH (60% in oil, 3.793 g, 94.83 mmol) at 0° C., and the mixture was stirred at rt for 30 min. 1-(Chloromethyl)-4-methoxybenzene (14.85 g, 94.83 mmol) followed by KI (1.312 g, 7.903 mmol) were then added, and the mixture was stirred at rt for 4 h. After completion of reaction, the reaction was quenched with ice and diluted with water (500 mL). The mixture was extracted with EtOAc (3×400 mL). The combined organic layer was washed with cold water (2×500 mL) and brine (2×500 mL). The organic layer was dried over Na₂SO₄, filtered and concentrated under reduced pressure to give a semi pure compound that was purified by silica gel column chromatography eluting with 13% EtOAc in PE to afford 4-bromo-1-(4-methoxybenzyl)-3-(((4-methoxybenzyl)oxy)methyl)-5-methyl-1H-pyrazole (22 g, 51.16 mmol, 64%) as a pale yellow solid. MS (ESI) m/z 433.06 [M+2H]⁺. ¹H NMR (400 MHz, CDCl₃) δ 7.33-7.29 (m, 2H), 7.08-7.05 (dd, J=2.0 Hz and 6.8 Hz, 2H), 6.90-6.83 (m, 4H), 5.22 (s, 2H), 4.51 (d, J=7.6 Hz, 4H), 3.80 (s, 3H), 3.78 (s, 3H), 2.16 (s, 3H)

### 1-(4-methoxybenzyl)-3-(((4-methoxybenzyl)oxy)methyl)-5-methyl-4-(4,4,5,5-tetramethyl-1,3,2-dioxaborolan-2-yl)-1H-pyrazole (5)

A suspension of 4-bromo-1-(4-methoxybenzyl)-3-(((4-methoxybenzyl)oxy)methyl)-5-methyl-1H-pyrazole (5 g, 11.62 mmol) in DMA (150 mL) were added Bispinacolatodiboron (11.75 g, 46.48 mmol) and KOAc (3.985 g, 40.67 mmol). The resulting solution was degassed for 20 min followed by the addition of Pd[P(Cy)₃]₂Cl₂ (857 mg, 1.162 mmol) and again degassed for 10 min. The mixture was heated at 110° C. for 16 h, and then diluted with water (200 mL) and extracted with EtOAc (3×200 mL). The combined organic layer was washed with water (2×200 mL) and brine (200 mL), dried over anhydrous Na₂SO₄, filtered and concentrated under reduced pressure. The mixture was purified by silica gel column chromatography and eluted using 10% EtOAc in PE to afford 1-(4-methoxybenzyl)-3-(((4-methoxybenzyl)oxy)methyl)-5-methyl-4-(4,4,5,5-tetramethyl-1,3,2-dioxaborolan-2-yl)-1H-pyrazole (5, 5.1 g, 10.66 mmol, LCMS: 60%) as a pale yellow oil. MS (ESI) m/z 479.28 [M+1]⁺.

### Intermediate 6

### 5-(((4-methoxybenzyl)oxy)methyl)-1,3-dimethyl-4-(4,4,5,5-tetramethyl-1,3,2-dioxaborolan-2-yl)-1H-pyrazole (6)

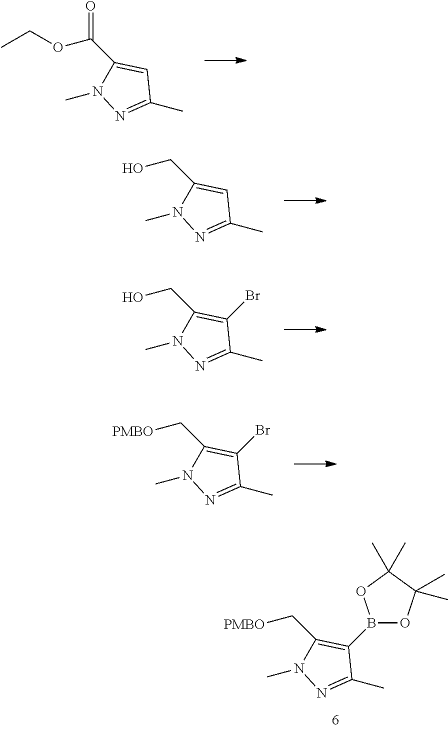

### (1,3-dimethyl-1H-pyrazol-5-yl)methanol

To a stirred solution of ethyl 1,3-dimethyl-1H-pyrazole-5-carboxylate (20 g, 118.98 mmol) in THF (200 mL) was added LiAlH₄ (2.4 M in THF, 148.7 mL, 356.95 mmol) at 0° C., and the mixture was stirred at RT for 1 h. After consumption of starting material, the reaction was quenched with ice cooled sat. Na₂SO₄ solution at 0° C. The resulting slurry was filtered through a Celite bed, which was then washed with ethyl acetate (2000 mL). The filtrate was dried over Na₂SO₄, filtered and evaporated to give a semi-pure compound that was washed with n-pentane (2×200 mL) and dried to afford (1,3-dimethyl-1H-pyrazol-5-yl)methanol (13 g, 103.04 mmol, 70%) as an off white solid. ¹H NMR (400 MHz, CDCl₃) δ 5.92 (s, 1H), 4.57 (s, 2H), 3.74 (s, 3H), 7.30 (t, J=7.6 Hz, 1H), 2.18 (s, 3H), 2.25 (s, 3H).

### (4-bromo-1,3-dimethyl-1H-pyrazol-5-yl)methanol

To a stirred solution of (1,3-dimethyl-1H-pyrazol-5-yl)methanol (13 g, 103.109 mmol) in DCM (190 mL) was added NBS (28 g, 154.66 mmol) portionwise over 30 mins at 0° C., and the mixture was stirred at rt for 1 h. After completion of reaction, the reaction was quenched with water (300 mL) and extracted with DCM (2×500 mL). The layers were separated and washed with brine (250 mL). The organic layer was dried over Na₂SO₄, filtered and evaporated to give a semi pure compound that was triturated with 20% EtOAc in PE to afford (4-bromo-1,3-dimethyl-1H-pyrazol-5-yl)methanol (16 g, 78.02 mmol, 70%) as a light yellow solid. ¹H NMR (400 MHz, CDCl₃) δ 4.65 (d, J=6.0 Hz, 2H), 3.85 (s, 3H), 2.21 (s, 3H), 2.08 (t, J=6.0 Hz, 1H).

### 4-bromo-5-(((4-methoxybenzyl)oxy)methyl)-1,3-dimethyl-1H-pyrazole

To a stirred solution of (4-bromo-1,3-dimethyl-1H-pyrazol-5-yl)methanol (15 g, 73.52 mmol) in DMF (150 mL) was added NaH (60% in oil, 4.5 g, 110.29 mmol) at 0° C., and the mixture was stirred at rt for 30 min. 1-(Chloromethyl)-4-methoxybenzene (10.91 mL, 80.872 mmol) followed by KI (2.4 g, 14.704 mmol) were added, and the mixture was stirred at rt for 2 h. After completion of reaction, the reaction was quenched with ice and diluted with water (500 mL), and then extracted with EtOAc (3×400 mL). The combined organic layer was washed with cold water (2×500 mL) and brine (2×500 mL). The organic layer was dried over Na₂SO₄, filtered and concentrated under reduced pressure to give a semi pure compound that was purified by silica gel column chromatography eluting with 10-20% EtOAc in PE to afford 4-bromo-5-(((4-methoxybenzyl)oxy)methyl)-1,3-dimethyl-1H-pyrazole (16 g, 49.198 mmol, 64%) as an off white solid. MS (LCMS) m/z 325.21 [M]⁺. ¹H NMR (400 MHz, CDCl₃) δ 7.25 (d, J=8.8 Hz, 2H), 6.88 (d, J=8.8 Hz, 2H), 4.50 (s, 2H), 4.42 (s, 2H), 3.81 (s, 3H), 3.80 (s, 3H), 2.22 (s, 3H).

### 5-(((4-methoxybenzyl)oxy)methyl)-1,3-dimethyl-4-(4,4,5,5-tetramethyl-1,3,2-dioxaborolan-2-yl)-1H-pyrazole (6)

To a stirred solution of 4-bromo-5-(((4-methoxybenzyl)oxy)methyl)-1,3-dimethyl-1H-pyrazole (4×5 g, 15.432 mmol) in THF (100 mL) was added n-BuLi (1.6 M in hexanes, 11.5 mL, 18.518 mmol) at −78° C., and the mixture was stirred at −78° C. for 50 min. 2-isopropoxy-4,4,5,5-tetramethyl-1,3,2-dioxaborolane (4.72 g, 23.148 mmol) was added at −78° C., and the mixture temperature was slowly raised to rt. The mixture was stirred for 4 h, and the solvents were evaporated. The residue was diluted with EtOAc (75 mL) and filtered through a Celite pad. The filtrate was evaporated to give a semi pure compound that was purified by silica gel column chromatography eluting with 30% EtOAc in PE to afford 5-(((4-methoxybenzyl)oxy)methyl)-1,3-dimethyl-4-(4,4,5,5-tetramethyl-1,3,2-dioxaborolan-2-yl)-1H-pyrazole (6, 6 g, 16.117 mmol, 26%) as a pale yellow solid. MS (LCMS) m/z 373.44 [M+H]⁺.

### Intermediate 7

### 3-(((3-((acetylthio)methyl)-1-methyl-1H-pyrazol-5-yl)methyl)thio)naphthalen-1-yl acetate (7)

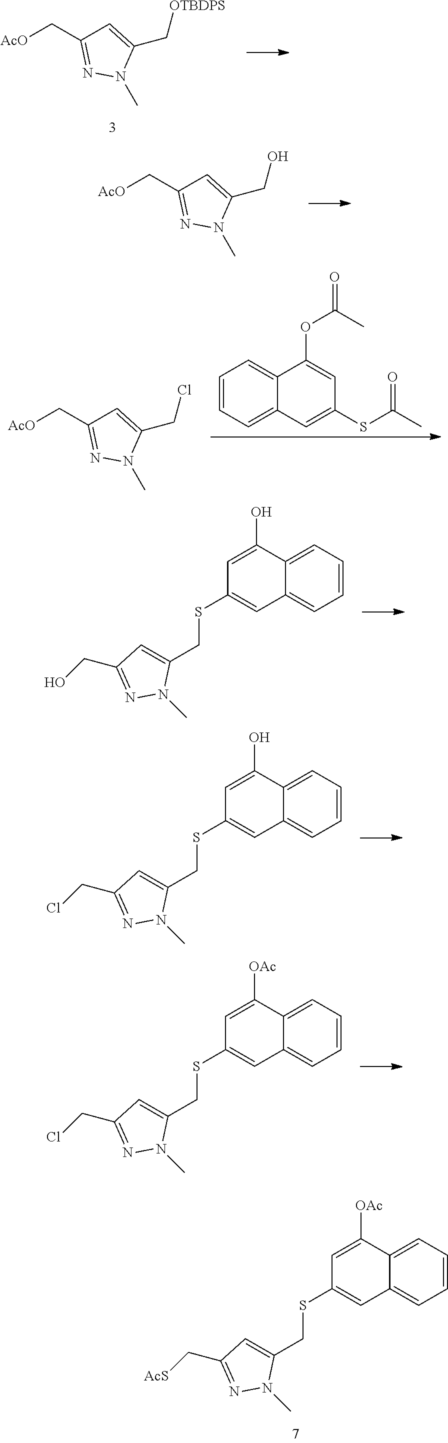

### (5-(hydroxymethyl)-1-methyl-1H-pyrazol-3-yl)methyl acetate

To a stirred solution of (5-(((tert-butyldiphenylsilyl)oxy)methyl)-1-methyl-1H-pyrazol-3-yl)methyl acetate (3) (2×110 g, 260.66 mmol) in THF (1.1 L) were added CsF (78 g, 521.32 mmol), followed by 1M TBAF in THF (104 mL, 104.26 mmol) at 0° C., and the mixture was stirred at rt for 4 h. After completion, the mixture was filtered to remove unwanted solid material, and the filtrate was concentrated to give a crude reside that was purified by silica gel column chromatography eluting with EtOAc afforded to give (5-(hydroxymethyl)-1-methyl-1H-pyrazol-3-yl)methyl acetate (62 g) as a pale yellow solid. MS (LCMS) m/z 185.07 [M+H]⁺. ¹H NMR (400 MHz, CDCl₃) δ 6.23 (s, 1H), 5.05 (s, 2H), 4.65 (s, 2H), 3.87 (s, 3H), 2.08 (s, 3H), 1.78 (bs, 1H).

### (5-(chloromethyl)-1-methyl-1H-pyrazol-3-yl)methyl acetate

To a stirred solution of (5-(chloromethyl)-1-methyl-1H-pyrazol-3-yl)methyl acetate (16 g, 86.9 mmol) in DCM (160 mL) under Ar was added SOCl₂ (12 mL, 173.9 mmol) at 0° C., and the mixture was stirred at rt for 30 min. The volatiles were evaporated and a crude residue was obtained. The reaction was quenched with a sat. NaHCO₃ solution (150 mL) and then extracted with DCM (2×150 mL). The combined organic layer was separated, dried over Na₂SO₄, filtered and concentrated to give semi pure (5-(chloromethyl)-1-methyl-1H-pyrazol-3-yl)methyl acetate (18 g) as a brown sticky solid that was used without any further purification. MS (LCMS) m/z 203.11 [M+H]⁺. ¹H NMR (400 MHz, CDCl₃) δ 6.45 (s, 1H), 5.17 (s, 2H), 4.59 (s, 2H), 4.15 (s, 3H), 2.13 (s, 3H).

### 3-(((3-(hydroxymethyl)-1-methyl-1H-pyrazol-5-yl)methyl)thio)naphthalen-1-ol

To a stirred degassed solution of semi pure (5-(chloromethyl)-1-methyl-1H-pyrazol-3-yl)methyl acetate (18 g, 89.1 mmol) in MeOH (90 mL) and THF (36 mL), were added K₂CO₃ (61.5 g, 445.5 mmol) followed by 3-(((3-(hydroxymethyl)-1-methyl-1H-pyrazol-5-yl)methyl)thio)naphthalen-1-ol (4, 23.16 g, 89.1 mmol), and the mixture was stirred at rt for 4 h. The mixture was filtered. The filtrate was concentrated to give a crude residue that was triturated with DCM (50 mL) and n-pentane (50 mL) to afford 3-(((3-(hydroxymethyl)-1-methyl-1H-pyrazol-5-yl)methyl)thio)naphthalen-1-ol (12 g, 40.0 mmol) as a pale brown solid. MS (LCMS) m/z 301.07 [M+H]⁺. ¹H NMR (400 MHz, DMSO) δ 8.05-8.03, (d, J=8.0 Hz, 1H), 7.31-7.29, (d, J=8.0 Hz, 1H), 7.11-7.07, (t, J=8.0 Hz, 1H), 6.92-6.88, (t, J=8.0 Hz, 1H), 6.30 (s, 1H), 6.12 (s, 1H), 5.95 (s, 1H), 4.28 (s, 2H), 3.74 (s, 3H), 3.19 (s, 2H).

### 3-(((3-(Chloromethyl)-1-methyl-1H-pyrazol-5-yl)methyl)thio)naphthalen-1-ol

To a stirred solution of 3-(((3-(hydroxymethyl)-1-methyl-1H-pyrazol-5-yl)methyl) thio)naphthalen-1-ol (40.0 g, 66.6 mmol) in DMF (200 mL) under Ar was added MeSO₂Cl (7.7 mL, 99.9 mmol) at 0° C. followed by LiCl (4.19 g, 99.9 mmol). The mixture was stirred at rt for 3 h. The reaction was quenched with ice cold water (300 mL) and then extracted with EtOAc (2×500 mL). The organic layer was washed with brine (500 mL), dried over Na₂SO₄, filtered and evaporated to afford semi pure 3-(((3-(chloromethyl)-1-methyl-1H-pyrazol-5-yl)methyl)thio)naphthalen-1-ol (35.0 g, 110.06 mmol) as a brown liquid that was used in the next step. MS (LCMS) m/z 319.17 [M+H]⁺. ¹H NMR (400 MHz, CDCl₃) δ 10.28 (bs, 1H), 8.05 (d, J=12 Hz, 1H), 7.73 (d, J=10 Hz, 1H), 7.50-7.34, (m, 3H), 6.81, (s, 1H), 6.25 (s, 1H), 6.18-6.10 (m, 1H), 4.57 (s, 2H), 4.37 (s, 2H), 3.80 (s, 3H).

### 3-(((3-(Chloromethyl)-1-methyl-1H-pyrazol-5-yl)methyl)thio)naphthalen-1-yl acetate

To a stirred solution of 3-(((3-(chloromethyl)-1-methyl-1H-pyrazol-5-yl)methyl)thio)naphthalen-1-ol crude (35.0 g) in acetonitrile (350 mL) were added DMAP (1.34 g, 10.95 mmol) and Ac₂O (16.75 mL, 164.3 mmol) at 0° C. The mixture was stirred at 30° C. for 3 h. After completion, the mixture was concentrated to give a pale brown crude reside, which was diluted with water (500 mL) and extracted with EtOAc (2×500 mL). The combined organic layers were washed with a brine solution (500 mL). The organic layer was separated, dried over Na₂SO₄, filtered and evaporated to get semi pure 3-(((3-(chloromethyl)-1-methyl-1H-pyrazol-5-yl)methyl)thio)naphthalen-1-yl acetate (32.2 g, 89.4 mmol) as a brown semi solid that was used in the next step. MS (LCMS) m/z 362.31 [M+H]⁺. ¹H NMR (400 MHz, CDCl3) δ 7.81 (m, 1H), 7.76 (m, 1H), 7.65 (s, 1H), 7.52 (m, 2H), 7.18 (s, 1H), 6.09, (s, 1H), 4.48 (s, 1H), 4.12 (s, 1H), 3.81 (s, 3H), 2.43 (s, 3H), 2.07 (s, 1H), 2.04 (s, 1H).

### 3-(((3-((Acetylthio)methyl)-1-methyl-1H-pyrazol-5-yl)methyl)thio)naphthalen-1-yl acetate (7)

To a stirred solution of 3-(((3-(chloromethyl)-1-methyl-1H-pyrazol-5-yl)methyl)thio)naphthalen-1-yl acetate (32.0 g, 88.64 mmol) dissolved in acetonitrile (320 mL) was added KSAc (15.15 g, 132.96 mmol) at 0° C. The mixture was allowed to stir at rt for 3 h. After completion, the mixture was concentrated to give a pale brown crude reside, which was diluted with water (300 mL) and extracted with EtOAc (2×500 mL). The organic layer was separated and dried over Na₂SO₄, filtered and evaporated to give semi pure compound 7 that was purified by Grace normal phase eluting with 30% EtOAc in PE to afforded 3-(((3-((acetylthio)methyl)-1-methyl-1H-pyrazol-5-yl)methyl)thio)naphthalen-1-yl acetate (7, 28.2 g, 70.5 mmol, 53% over 3 steps) as an off white solid. MS (LCMS) m/z 401.20 [M+H]⁺. ¹H NMR (400 MHz, CDCl₃) δ 7.79-7.75 (m, 1H), 7.64 (s, 1H), 7.20 (s, 1H), 7.54-7.51 (s, 1H), 7.20 (s, 1H), 5.99 (s, 2H), 4.15 (s, 2H), 4.09 (s, 2H), 3.78 (s, 3H), 2.46 (s, 3H), 2.29 (s, 3H).

### Intermediate 8

### 3-(((3-((acetylthio)methyl)-1-ethyl-1H-pyrazol-5-yl)methyl)thio)naphthalen-1-yl acetate (8)

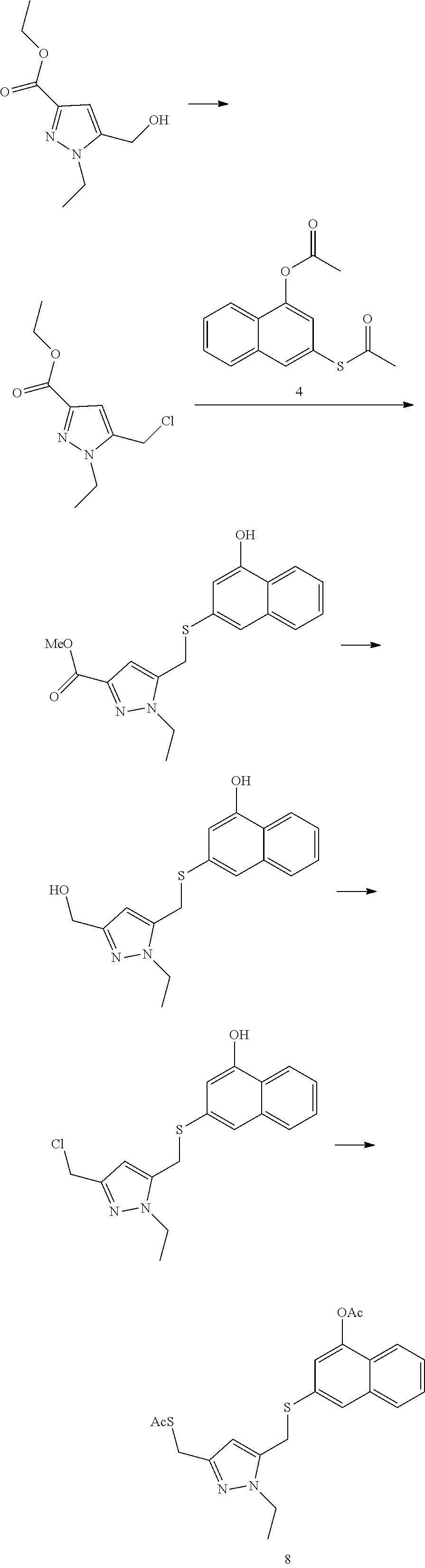

### Ethyl 5-(chloromethyl)-1-ethyl-1H-pyrazole-3-carboxylate

To a stirred solution of ethyl 1-ethyl-5-(hydroxymethyl)-1H-pyrazole-3-carboxylate (5 g, 25.2 mmol) in DCM (50 mL) under Ar was added SOCl₂ (2.2 mL, 30.3 mmol) at 0° C. The mixture was stirred at rt for 30 min. The volatiles were evaporated, and a crude residue was obtained. The reaction was quenched with a sat. NaHCO₃ solution (50 mL) and extracted with DCM (2×50 mL). The combined organic layer was separated, dried over Na₂SO₄, filtered and concentrated to give semi pure Ethyl 5-(chloromethyl)-1-ethyl-1H-pyrazole-3-carboxylate (5.0 g, 23.14 mmol, 91% yield for two batches) as a brown sticky solid that was used without further purification. MS (LCMS) m/z 217.12 [M+H]⁺. ¹H NMR (400 MHz, CDCl₃) δ 6.81 (s, 1H), 4.60 (s, 2H), 4.40 (q, J=6.8, 2H), 4.29 (q, J=7.2 Hz, 2H), 1.53 (t, 3H), 1.38 (t, 3H).

### Methyl 1-ethyl-5-(((4-hydroxynaphthalen-2-yl)thio)methyl)-1H-pyrazole-3-carboxylate

To a stirred degassed solution of semi pure Ethyl 5-(chloromethyl)-1-ethyl-1H-pyrazole-3-carboxylate (5 g, 23.1 mmol) in MeOH (50 mL) was added K₂CO₃ (7.66 g, 55.5 mmol) followed by 3-(acetylthio)naphthalen-1-yl acetate (4) (6.018 g, 23.1 mmol). The mixture was stirred at rt for 16 h. The volatiles were evaporated. A crude residue was obtained. The mixture was diluted with water (20 mL) and extracted with EtOAc (2×50 mL). The combined organic layer was separated, dried over Na₂SO₄, filtered and concentrated. The crude material was purified by silica gel (100-200) using 30% EtOAc in PE to afford methyl 1-ethyl-5-(((4-hydroxynaphthalen-2-yl)thio)methyl)-1H-pyrazole-3-carboxylate (6 g, 17.54 mmol, 75% for two batches) as a pale brown solid. MS (LCMS) m/z 343.26 [M+H]⁺.

### 3-(((1-ethyl-3-(hydroxymethyl)-1H-pyrazol-5-yl)methyl)thio)naphthalen-1-ol

To a stirred solution methyl 1-ethyl-5-(((4-hydroxynaphthalen-2-yl)thio)methyl)-1H-pyrazole-3-carboxylate (2×3.5 g, 20.4 mmol) in THF (35 mL) was added LIAlH₄ (2.4 M in THF, 4.2 mL, 20.4 mmol) at 0° C., and the mixture was stirred at rt for 1 h. After completion of the reaction, the mixture was cooled to 0° C. The reaction was quenched with a sat. Na₂SO₄ solution. The mixture was filtered through a Celite bed that was then washed with 10% MeOH in DCM. The filtrate was evaporated to give a semi pure compound that was purified by silica gel (100-200) using 60% EtOAc in PE to afford 3-(((1-ethyl-3-(hydroxymethyl)-1H-pyrazol-5-yl)methyl)thio)naphthalen-1-ol (4 g, 12.7 mmol, 70% for two batches) as a brown sticky solid. MS (LCMS) m/z 313.25 [M−H]⁺.

### 3-(((3-(chloromethyl)-1-ethyl-1H-pyrazol-5-yl)methyl)thio)naphthalen-1-ol

To a stirred solution of 3-(((1-ethyl-3-(hydroxymethyl)-1H-pyrazol-5-yl)methyl)thio)naphthalen-1-ol (2×2 g, 6.3 mmol) in DCM:DMF (7:1, 16 mL) under Ar was added SOCl₂ (0.46 mL, 6.3 mmol) at 0° C. The mixture was stirred at rt for 30 min. The volatiles were evaporated, and then added MeOH (20 mL). The mixture was stirred for 1 h at rt. Water (20 mL) was added to the mixture, and then methanol was evaporated. The mixture was extracted with DCM (2×30 mL). The combined organic layer was separated, dried over Na₂SO₄, filtered and concentrated to give semi pure 3-(((3-(chloromethyl)-1-ethyl-1H-pyrazol-5-yl)methyl)thio)naphthalen-1-ol (2 g, 6.02 mmol, 95% for two batches) as a brown sticky solid that was used in the next step without further purification. MS (LCMS) m/z 333.59 [M+H]⁺.

### 3-(((3-((acetylthio)methyl)-1-ethyl-1H-pyrazol-5-yl)methyl)thio)naphthalen-1-yl acetate (8)

To a stirred solution of 3-(((3-(chloromethyl)-1-ethyl-1H-pyrazol-5-yl)methyl)thio)naphthalen-1-ol (10 g, 30.1 mmol) in acetonitrile (100 mL) were added DMAP (368 mg, 3.01 mmol) and Ac₂O (3.41 mL, 36.1 mmol) at 0° C. The mixture was stirred at rt for 1 h. The solvent was evaporated, diluted with ice cold water (40 mL) and extracted with EtOAc (2×50 mL). The organic layer was separated, dried over Na₂SO₄, filtered and evaporated to give a brown sticky solid. The mixture was dissolved in acetonitrile (100 mL) and KSAc (4.58 g, 40.01 mmol) was added at rt. The mixture was stirred at rt for 16 h. The solvent was evaporated, diluted with ice cold water (80 mL) and extracted with EtOAc (2×100 mL). The organic layer was separated, dried over Na₂SO₄, filtered and evaporated. The crude was purified by silica gel (100-200) using 50% EtOAc in PE to afford 3-(((3-((acetylthio)methyl)-1-ethyl-1H-pyrazol-5-yl)methyl)thio)naphthalen-1-yl acetate (8, 7.5 g, 18.11 mmol, 68% for two batches for two steps) as a brown gummy liquid. MS (LCMS) m/z 415.37 [M+H]⁺. ¹H NMR (400 MHz, CDCl3) δ 7.82-7.75 (m, 2H), 7.63 (s, 1H), 7.52-7.49 (m, 2H), 7.20 (d, J=1.6 Hz, 1H), 5.99 (s, 1H), 4.11-4.04 (m, 6H), 2.46 (s, 3H), 2.29 (s, 3H), 1.43 (t, 3H).

### Intermediate 9

### 3-(((3-((acetylthio)methyl)-1-isopropyl-1H-pyrazol-5-yl)methyl)thio)naphthalen-1-yl acetate (9)

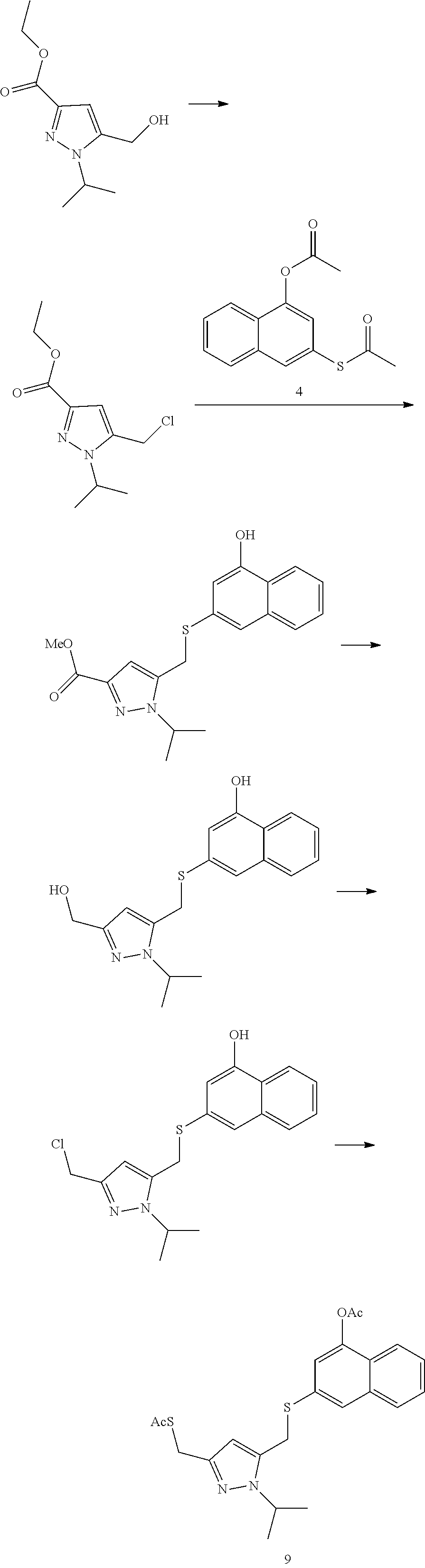

### Ethyl 5-(chloromethyl)-1-isopropyl-1H-pyrazole-3-carboxylate

To a stirred solution of ethyl 5-(hydroxymethyl)-1-isopropyl-1H-pyrazole-3-carboxylate (2×7 g, 32.979 mmol) in DCM (90 mL) under Ar was added SOCl₂ (2.6 mL, 36.277 mmol) at 0° C., and the mixture was stirred at rt for 1 h. The mixture was diluted with DCM (500 mL) and washed with a sat. NaHCO₃ solution (3×200 mL). The organic layer was separated, dried over Na₂SO₄, filtered and evaporated to afford semi pure methyl ethyl 5-(chloromethyl)-1-isopropyl-1H-pyrazole-3-carboxylate (14.5 g, 62.854 mmol) as an off-white solid that was used in next step without further purification. MS (LCMS) m/z 231.05 [M+H]⁺. ¹H NMR (400 MHz, CDCl₃) δ 6.77 (s, 1H), 4.67-4.60 (m, 3H), 4.41-4.35 (m, 2H), 1.60-1.55 (d, J=6.4 Hz, 6H), 1.38 (t, J=7.2 Hz, 3H).

### Methyl 5-(((4-hydroxynaphthalen-2-yl)thio)methyl)-1-isopropyl-1H-pyrazole-3-carboxylate

To a stirred solution of ethyl 5-(chloromethyl)-1-isopropyl-1H-pyrazole-3-carboxylate (2×7 g, 30.343 mmol) in MeOH (60 mL), were added K₂CO₃ (10 g, 72.824 mmol), and the mixture was degassed with Ar for 10 min. In another round bottom flask, 3-(acetylthio)naphthalen-1-yl acetate (4) (8.68 g, 33.878 mmol) in methanol (15 mL) was degassed with Ar for 10 min, and this solution was added to previous mixture dropwise. The mixture was stirred at rt for 16 h, and the solvent was evaporated. The mixture was diluted with water (200 mL) and extracted with EtOAc (2×500 mL). The organic layer was separated, dried over Na₂SO₄, filtered and evaporated to give semi pure compound that was purified by silica gel column chromatography using 20% EtOAC in PE to afford methyl 5-(((4-hydroxynaphthalen-2-yl)thio)methyl)-1-isopropyl-1H-pyrazole-3-carboxylate (18 g, 50.499 mmol, 77% over two steps) as a light brown solid. MS (LCMS) m/z 357.07 [M+H]⁺. ¹H NMR (400 MHz, CDCl₃) δ 8.13 (d, J=8.0 Hz, 1H), 7.72-7.68 (m, 1H), 7.52-7.48 (m, 2H), 7.39 (s, 1H), 6.73 (s, 1H), 6.61 (s, 1H), 5.91 (br s, 1H), 4.65-4.60 (m, 1H), 4.15 (s, 2H), 3.87 (s, 3H), 1.54 (d, J=6.8 Hz, 6H).

### 3-(((3-(hydroxymethyl)-1-isopropyl-1H-pyrazol-5-yl)methyl)thio)naphthalen-1-ol

To a stirred solution of methyl 5-(((4-hydroxynaphthalen-2-yl)thio)methyl)-1-isopropyl-1H-pyrazole-3-carboxylate (2×9 g, 25.249 mmol) in THF (100 mL) was added LiAlH₄ (1 M in THF, 37.8 mL, 37.8 mmol) at 0° C., and the mixture was stirred at rt for 1 h. After consumption of starting material, the reaction was quenched with an ice cooled sat. Na₂SO₄ solution at 0° C. The resulting slurry was filtered on a Celite bed that was then washed with ethyl acetate (1000 mL). The filtrate was dried over Na₂SO₄, filtered and evaporated to give semi pure compound that was washed with n-pentane (200 mL×2) and dried to afford 3-(((3-(hydroxymethyl)-1-isopropyl-1H-pyrazol-5-yl)methyl)thio)naphthalen-1-ol (12 g, 36.537 mmol, 91%) as a brown solid. MS (LCMS) m/z 329.25 [M+H]⁺.

### 3-(((3-(chloromethyl)-1-isopropyl-1H-pyrazol-5-yl)methyl)thio)naphthalen-1-ol

To a stirred solution of 3-(((3-(hydroxymethyl)-1-isopropyl-1H-pyrazol-5-yl)methyl)thio)naphthalen-1-ol (2×6 g, 18.269 mmol) in DCM (70 mL) under Ar was added SOCl₂ (1.5 mL, 21.923 mmol) at 0° C., and the mixture was stirred at rt for 1 h. The mixture was diluted with DCM (250 mL) and washed with a sat. NaHCO₃ solution (3×100 mL). The organic layer was separated, dried over Na₂SO₄, filtered and evaporated to afford semi pure 3-(((3-(chloromethyl)-1-isopropyl-1H-pyrazol-5-yl)methyl)thio)naphthalen-1-ol (13 g, 37.478 mmol) as a brown semi solid that was used in next step without further purification. MS (LCMS) m/z 347.22 [M+H]⁺.

### 3-(((3-((acetylthio)methyl)-1-isopropyl-1H-pyrazol-5-yl)methyl)thio)naphthalen-1-yl acetate(9)

To a stirred solution of 3-(((3-(chloromethyl)-1-isopropyl-1H-pyrazol-5-yl)methyl)thio)naphthalen-1-ol (2×6.5 g, 18.739 mmol) in acetonitrile (90 mL), were added Ac₂O (2.2 g, 22.486 mmol) and DMAP (230 mg, 1.8739 mmol) at 15° C.-20° C. The mixture was stirred at the same temp for 3 h. After consumption of starting material, the solvent was evaporated. The residue was diluted with water (200 mL) and extracted with EtOAc (2×500 mL). The organic layer was separated, dried over Na₂SO₄, filtered and evaporated to give a semi pure compound which was dissolve in CH₃CN were added KSAc (6.8 g, 60.3.92 mmol) at 15° C.-20° C. and reaction mixture was stirred at same temp for 3 h. After consumption of starting material, the solvent was evaporated. The residue was diluted with water (200 mL) and extracted with EtOAc (2×500 mL). The organic layer was separated, dried over Na₂SO₄, filtered and evaporated to give a semi pure compound that was purified by silica gel column chromatography using 20% EtOAc in PE to afford 3-(((3-((acetylthio)methyl)-1-isopropyl-1H-pyrazol-5-yl)methyl)thio)naphthalen-1-yl acetate (9, 6 g, 14.00 mmol, 40% over two steps) as a light brown solid. MS (LCMS) m/z 429.13.

### Intermediate 10

### 3-(((3-((acetylthio)methyl)-1-(cyclopropylmethyl)-1H-pyrazol-5-yl)methyl)thio)naphthalen-1-yl acetate (10)

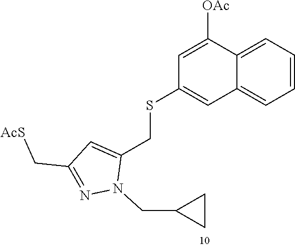

3-(((3-((acetylthio)methyl)-1-(cyclopropylmethyl)-1H-pyrazol-5-yl)methyl)thio)naphthalen-1-yl acetate (10) was prepared by the same procedure of intermediate 9 starting from ethyl 1-(cyclopropylmethyl)-5-(hydroxymethyl)-1H-pyrazole-3-carboxylate. MS (LCMS) m/z 441.47 [M+H]⁺. ¹H NMR (400 MHz, CDCl₃) δ 7.85-7.80 (m, 1H), 7.80-7.75 (m, 1H), 7.63 (s, 1H), 7.55-7.45 (m, 2H), 7.20 (s, 1H), 6.02 (s, 1H), 4.14 (s, 2H), 4.05 (s, 2H), 3.95 (d, J=9.2 Hz, 2H), 2.47 (s, 3H), 2.29 (s, 3H), 1.30-1.20 (m, 1H), 0.60-0.50 (m, 2H), 0.40-0.35 (m, 2H).

### Intermediate 11

### 3-(((3-((acetylthio)methyl)-1-isobutyl-1H-pyrazol-5-yl)methyl)thio)naphthalen-1-yl acetate (11)

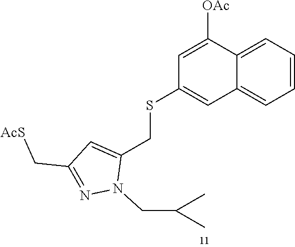

3-(((3-((acetylthio)methyl)-1-isobutyl-1H-pyrazol-5-yl)methyl)thio)naphthalen-1-yl acetate (11) was prepared by the same procedure of intermediate 9 starting from ethyl 5-(hydroxymethyl)-1-isobutyl-1H-pyrazole-3-carboxylate. MS (LCMS) m/z 443.47 [M+H]⁺. ¹H NMR (400 MHz, CDCl3) δ 7.82-7.75 (m, 2H), 7.62 (s, 1H), 7.52-7.49 (m, 2H), 7.20 (d, J=1.6 Hz, 1H), 6.00 (s, 1H), 4.10 (s, 2H), 4.04 (s, 2H), 3.84 (d, J=7.2 Hz, 2H), 2.47 (s, 3H), 2.28 (s, 3H), 2.27-2.20 (m, 1H), 0.90 (d, J=6.4 Hz, 6H).

### Intermediate 12

### 3-(((3-((acetylthio)methyl)-1-(4-methoxybenzyl)-1H-pyrazol-5-yl)methyl)thio)naphthalen-1-yl acetate (12)

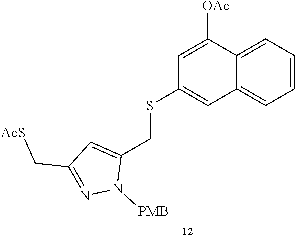

3-(((3-((acetylthio)methyl)-1-(4-methoxybenzyl)-1H-pyrazol-5-yl)methyl)thio)naphthalen-1-yl acetate (12) was prepared by the same procedure of intermediate 9 starting from ethyl 5-(hydroxymethyl)-1-(4-methoxybenzyl)-1H-pyrazole-3-carboxylate. MS (LCMS) m/z 507.51. ¹H NMR (400 MHz, CDCl₃) δ 7.81-7.79 (m, 1H), 7.73-7.70 (m, 1H), 7.53-7.48 (m, 3H), 7.14 (d, J=1.6 Hz, 1H), 7.14 (dd, J=6.8 Hz, 2 Hz, 2H), 6.81 (dd, J=6.8 Hz, 2 Hz, 2H), 6.05 (s, 1H), 5.29 (d, J=4 Hz, 2H), 4.06 (s, 2H), 3.96 (s, 2H), 3.76 (s, 3H), 2.46 (s, 3H), 2.29 (s, 3H).

### Intermediate 13

### 1-(Bicyclo[1.1.1]pentan-1-yl)-5-(((4-methoxybenzyl)oxy)methyl)-3-methyl-4-(4,4,5,5-tetramethyl-1,3,2-dioxaborolan-2-yl)-1H-pyrazole (13)

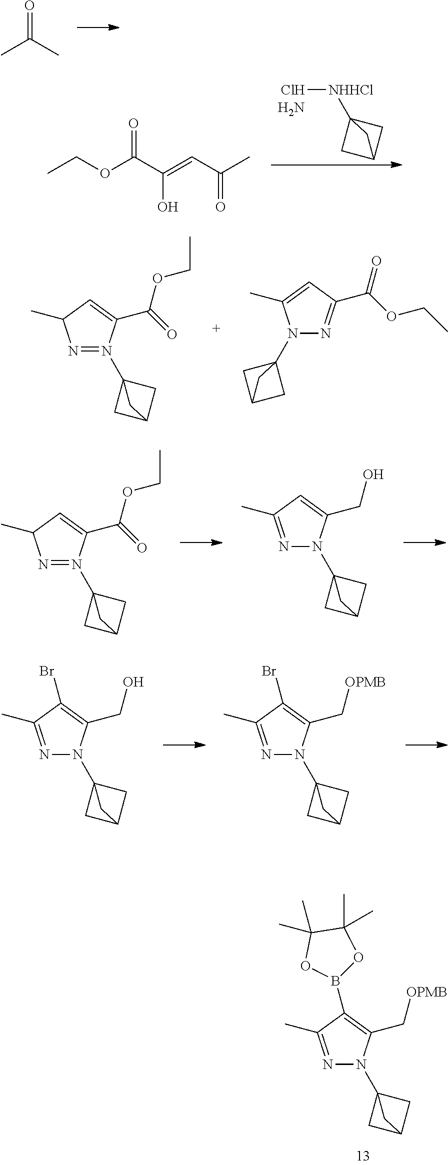

### Ethyl (Z)-2-hydroxy-4-oxopent-2-enoate

To a stirred solution of acetone (2×25 g, 430 mmol) in THF (250 mL) was added diethyl oxalate (62.9 g, 430 mmol) and KtOBu (48 g, 430 mmol) at 0° C., and the mixture was allowed to stir at rt for 16 h. The mixture was diluted with EtOAc (1000 mL), and the reaction quenched with 2N HCl (1000 mL). The mixture was extracted with EtOAc (2×2000 mL). The combined organic layer was washed with brine (1000 mL), dried over Na₂SO₄, filtered and evaporated. The crude was purified by silica gel column chromatography using 2% EtOAc in PE to afford ethyl (Z)-2-hydroxy-4-oxopent-2-enoate (65 g, 411 mmol, 47%) as a brown liquid. MS (LCMS) m/z 159.00 [M+H]⁺. ¹H NMR (400 MHz, CDCl₃) δ 14.45 (bs, 1H), 6.37 (s, 1H), 4.38-4.32 (m, 2H), 2.26 (s, 3H), 1.40-1.36 (m, 2H).

### Ethyl 1-(bicyclo[1.1.1]pentan-1-yl)-3-methyl-1H-pyrazole-5-carboxylate

To a stirred solution of ethyl (Z)-2-hydroxy-4-oxopent-2-enoate (40 g, 258 mmol) in ethanol (2200 mL) was added N-(bicyclo[1.1.1]pentan-1-yl)-λ³-chloranediamine dihydrochloride (22 g, 129 mmol) at 0° C., and the mixture was stirred at 80° C. for 2 h. After completion of the reaction, the solvent was evaporated. The mixture was diluted with water (500 mL) and extracted with EtOAc (3×750 mL). The organic layer was separated, dried over Na₂SO₄, filtered and evaporated to give a semi pure compound and another stereoisomer that was purified by silica gel (100-200 mesh) column chromatography using 5-20% EtOAc in PE to afford ethyl 1-(bicyclo[1.1.1]pentan-1-yl)-3-methyl-1H-pyrazole-5-carboxylate (10 g, 45.45 mmol, 35%) and another product (ethyl 1-(bicyclo[1.1.1]pentan-1-yl)-5-methyl-1H-pyrazole-3-carboxylate (20 g, LCMS: 48%)). Ethyl 1-(bicyclo[1.1.1]pentan-1-yl)-3-methyl-1H-pyrazole-5-carboxylate: MS (LCMS) m/z 221.18 [M+H]⁺. ¹H NMR (400 MHz, CDCl₃) δ 6.62 (s, 1H), 4.37-4.28 (m, 2H), 2.55 (s, 1H), 2.42 (s, 6H), 2.26 (s, 3H), 1.38-1.34 (m, 3H).

### (1-(Bicyclo[1.1.1]pentan-1-yl)-3-methyl-1H-pyrazol-5-yl)methanol

To a stirred solution of ethyl 1-(bicyclo[1.1.1]pentan-1-yl)-3-methyl-1H-pyrazole-5-carboxylate (2×4.5 g, 20.44 mmol) in THF (45 mL) was added LiAlH₄ (2.4 M in THF, 2.6 mL, 20.44 mmol) at 0° C., and the mixture was stirred at rt for 2 h. The reaction was quenched with an ice cooled sat. Na₂SO₄ solution. The resulting slurry was filtered on a Celite bed that was washed with EtOAc (4×200 mL). Both filtrates were mixed, dried over Na₂SO₄, filtered and evaporated to give (1-(bicyclo[1.1.1]pentan-1-yl)-3-methyl-1H-pyrazol-5-yl)methanol (7.3 g, 40.78 mmol, 100%) as a clear viscous liquid that was used for the next step without further purification. MS (LCMS) m/z 179.37 [M+H]⁺. ¹H NMR (400 MHz, CDCl₃) δ 5.99 (s, 1H), 4.67 (d, J=6.0 Hz, 2H), 2.57 (s, 1H), 2.38 (s, 6H), 2.24 (s, 3H).

### (1-(Bicyclo[1.1.1]pentan-1-yl)-4-bromo-3-methyl-1H-pyrazol-5-yl)methanol

To a stirred solution of get (1-(bicyclo[1.1.1]pentan-1-yl)-3-methyl-1H-pyrazol-5-yl)methanol (7.3 g, 41.01 mmol) in DCM (100 mL) was added NBS (7.3 g, 41.01 mmol) at 0° C., and the mixture was stirred at rt for 2 h. The mixture was diluted with water (100 mL) and extracted with DCM (3×100 mL). The organic layer was separated, dried over Na₂SO₄, filtered and evaporated to give a semi pure compound that was triturated with pentane:ether (1:1) (3×50 mL) to give (1-(bicyclo[1.1.1]pentan-1-yl)-4-bromo-3-methyl-1H-pyrazol-5-yl)methanol (8 g, 30.88 mmol, 75%) as a pale yellow solid that was used for the next step. ¹H NMR (400 MHz, CDCl₃) δ 4.44 (s, 2H), 2.56 (s, 1H), 2.28 (s, 6H), 2.09 (s, 3H).

### 1-(Bicyclo[1.1.1]pentan-1-yl)-4-bromo-5-(((4-methoxybenzyl)oxy)methyl)-3-methyl-1H-pyrazole

To a stirred solution of (1-(bicyclo[1.1.1]pentan-1-yl)-4-bromo-3-methyl-1H-pyrazol-5-yl)methanol (2×4 g, 15.56 mmol) in DMF (80 mL) was added NaH (60% in oil, 0.933 g, 38.91 mmol) at 0° C., and the mixture was stirred at rt for 20 min. 1-(Chloromethyl)-4-methoxybenzene (3.64 g, 23.33 mmol) and NaI (0.46 g, 3.11 mmol) were added, and the mixture was stirred at rt for 2 h. After completion of reaction, the reaction was quenched ice water and diluted with water (200 mL), and then extracted with EtOAc (3×200 mL). The combined organic layers were washed with water (2×200 mL) and brine (200 mL), dried over Na₂SO₄ and concentrated under reduced pressure to give a semi pure compound that was purified by silica gel column chromatography eluting with 7% EtOAc/PE to afford 1-(bicyclo[1.1.1]pentan-1-yl)-4-bromo-5-(((4-methoxybenzyl)oxy)methyl)-3-methyl-1H-pyrazole (10 g, 26.65 mmol, 85%) as a pale yellow oil. MS (LCMS) m/z 377.12 [M+H]⁺. ¹H NMR (400 MHz, CDCl₃) δ 7.27-7.23 (m, 2H), 6.90-6.85 (m, 2H), 4.54 (s, 2H), 4.42 (s, 2H), 3.81 (s, 3H), 2.53 (s, 1H), 2.34 (s, 6H), 2.24 (s, 3H).

### 1-(Bicyclo[1.1.1]pentan-1-yl)-5-(((4-methoxybenzyl)oxy)methyl)-3-methyl-4-(4,4,5,5-tetramethyl-1,3,2-dioxaborolan-2-yl)-1H-pyrazole (13)

A suspension of 1-(bicyclo[1.1.1]pentan-1-yl)-4-bromo-5-(((4-methoxybenzyl)oxy)methyl)-3-methyl-1H-pyrazole (2×5 g, 13.26 mmol), bis pinacolato diboron (13.42 g, 53.05 mmol) and KOAc (4.54 g, 46.41 mmol) was degassed for 20 min followed by the addition of Pd[P(Cy)₃]₂Cl₂ (489 mg, 0.66 mmol). The mixture was degassed again for 10 min in DMA (100 mL). The mixture was heated at 110° C. for 4 h. The mixture was diluted with water (200 mL) and extracted with EtOAc (3×200 mL). The combined organic layers were washed with water (2×200 mL) and brine (200 mL), dried over Na₂SO₄ and concentrated under reduced pressure to give semi pure 13 that was purified by silica gel column chromatography eluting using 10% EtOAc in PE to afford 1-(bicyclo[1.1.1]pentan-1-yl)-5-(((4-methoxybenzyl)oxy)methyl)-3-methyl-4-(4,4,5,5-tetramethyl-1,3,2-dioxaborolan-2-yl)-1H-pyrazole (13, 10 g, 23.35 mmol, 86%) as a pale yellow oil. MS (LCMS) m/z 425.28 [M+H]⁺. ¹H NMR (400 MHz, CDCl₃) δ 7.25-7.22 (m, 2H), 6.90-6.84 (m, 2H), 4.76 (s, 2H), 4.42 (s, 2H), 3.81 (s, 3H), 2.52 (s, 1H), 2.36 (s, 9H), 1.26 (s, 12H).

### Intermediate 14

### Methyl 7-bromo-6-fluoro-3-(3-methoxy-3-oxopropyl)-1H-indole-2-carboxylate (14)

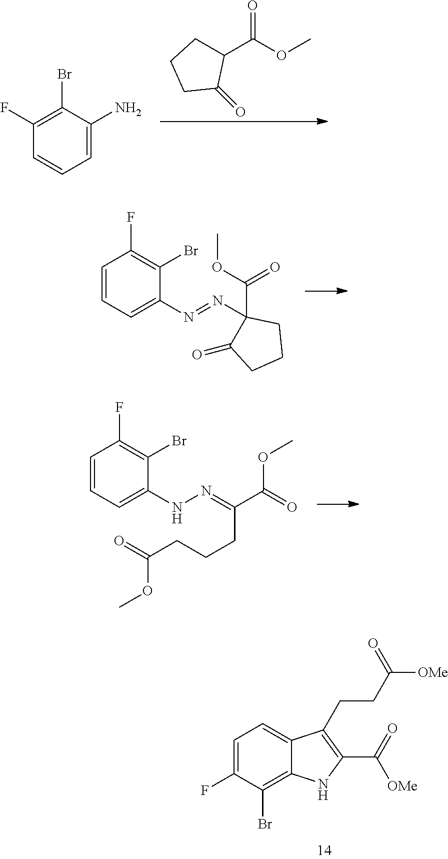

### Methyl (E)-1-((2-bromo-3-fluorophenyl)diazenyl)-2-oxocyclopentane-1-carboxylate

To a stirred solution of 2-bromo-3-fluoroaniline (50 g, 265 mmol, 1.0 eq.) in con. HCl (124 mL) was added water (684 mL), and the mixture was stirred at rt for 16 h. A solution of NaNO₂ (19 g, 278 mmol, 1.05 eq.) in water (30 mL) was added at 0° C., and the mixture stirred at rt for 1.5 h. A solution of KOAc (363 g, 3710 mmol, 14 eq.) in water (514 mL) and methyl 2-oxocyclopentane-1-carboxylate (38 g, 268 mmol, 1.01 eq.) was added dropwise. The mixture was stirred at 0-5° C. for 0.5 h and then at rt for 2 h. After completion, the mixture was diluted with water and extracted with DCM (3×500 mL). The combined organic layer was washed with brine (200 mL), dried over Na₂SO₄, filtered and evaporated to afford methyl (E)-1-((2-bromo-3-fluorophenyl)diazenyl)-2-oxocyclopentane-1-carboxylate (70 g) as a brown solid that was used as such for the next step. MS (LCMS) m/z 365.05 [M+Na]⁺. ¹H NMR (400 MHz, CDCl₃) δ 7.35-7.30 (m, 1H), 7.24-7.19 (m, 2H) 3.88 (s, 3H), 2.83-2.78 (m, 1H), 2.76-2.72 (m, 1H), 2.50-2.48 (m, 2H), 2.16-2.10 (m, 2H).

### Dimethyl (E/Z)-2-(2-(2-bromo-3-fluorophenyl)hydrazono)hexanedioate

To a stirred solution of methyl (E)-1-((2-bromo-3-fluorophenyl)diazenyl)-2-oxocyclopentane-1-carboxylate (70 g, 204 mmol) in MeOH (700 mL) was added con. H₂SO₄ (104 mL, 985 mmol) at 0° C., and the mixture was stirred at 80° C. for 2 h. The mixture was cooled to rt. The precipitated solids were filtered and washed with MeOH to afford dimethyl (E/Z)-2-(2-(2-bromo-3-fluorophenyl)hydrazono)hexanedioate (68 g, 181.3 mmol) as an off white solid that was used without further purification. MS (LCMS) m/z 375.10 [M+H]⁺. ¹H NMR (400 MHz, CDCl₃) δ 12.44 (s, 1H), 7.37 (d, J=10.4 Hz, 1H), 7.25-7.19 (m, 1H), 7.55 (dt, J=8.4, 1.6 Hz, 1H), 3.87 (s, 3H), 3.67 (s, 3H), 2.60 (t, J=7.6 Hz, 2H), 2.41 (t, J=7.2 Hz, 2H), 2.00-1.97 (m, 2H).

### Methyl 7-bromo-6-fluoro-3-(3-methoxy-3-oxopropyl)-1H-indole-2-carboxylate (14)

To a stirred solution of dimethyl (E/Z)-2-(2-(2-bromo-3-fluorophenyl)hydrazono)hexanedioate (68 g, 181 mmol, 1.0 eq.) in MeOH (680 mL) was added con. H₂SO₄ (121 mL, 2290 mmol, 12.65 eq.) at 0° C., and the mixture was stirred at 80° C. for 4 days. The mixture was cooled to rt. The precipitated solids were filtered, washed with MeOH and dried to afford methyl 7-bromo-6-fluoro-3-(3-methoxy-3-oxopropyl)-1H-indole-2-carboxylate (14, 56 g, 156.8 mmol, 59% over 3 steps) as an off white solid. MS (LCMS) m/z 358.07, 360.08 [M+H, M+3H]⁺. ¹H NMR (400 MHz, CDCl₃) δ 8.83 (br s, 1H), 8.22 (br s, 1H), 7.64-7.60 (m, 1H), 6.99 (t, J=8.8 Hz, 1H), 3.98 (s, 3H), 3.77 (s, 3H), 3.49 (s, 1H), 3.37 (t, J=2.0 Hz, 1H), 2.68 (t, J=5.2 Hz, 2H).

### Intermediate 15

### S-((5-(((4-(allyloxy)naphthalen-2-yl)thio)methyl)-1-methyl-1H-pyrazol-3-yl)methyl) ethanethioate (15)

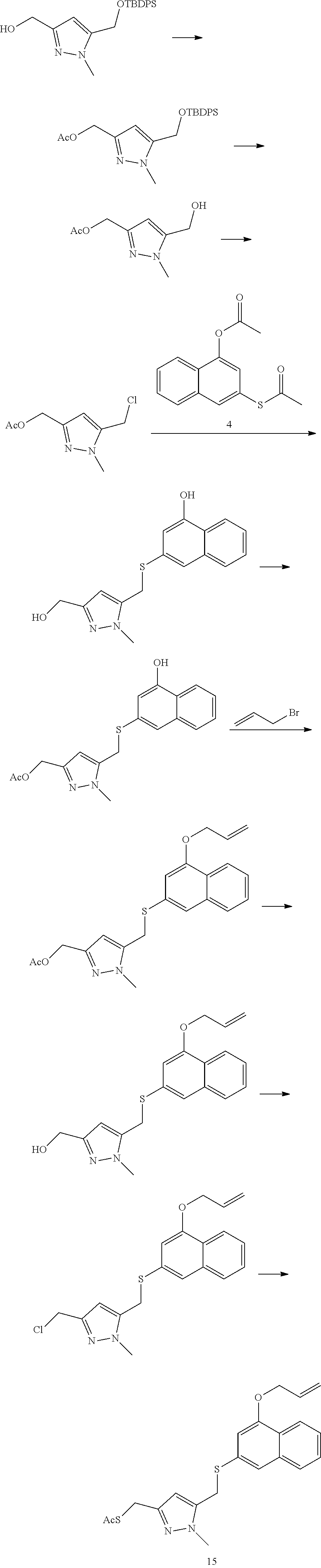

### (5-(((tert-butyldiphenylsilyl)oxy)methyl)-1-methyl-1H-pyrazol-3-yl)methyl acetate

To a stirred solution of (5-(((tert-butyldiphenylsilyl)oxy)methyl)-1-methyl-1H-pyrazol-3-yl)methyl acetate (230 g, 605.26) in DCM (2.3 L) were added Et₃N (170 mL, 1210.52 mmol), Cat. DMAP (5 g), and Ac₂O (68 mL, 726.31 mmol) at 0° C., and the mixture was stirred at rt for 6 h. After completion, the mixture was diluted with water (2 L) and extracted with DCM (2×1 L). The combined organic layer was washed with brine (1 L). The organic layer was separated, dried over Na₂SO₄, filtered and evaporated to a semi pure compound (220 g) as a pale yellow liquid that was used without any further purification. MS (LCMS) m/z 423.16 [M+H]⁺. ¹H NMR (400 MHz, CDCl₃) δ 7.66-7.64 (m, 4H), 7.47-7.26 (m, 6H), 5.29 (s, 1H), 5.04 (s, 2H), 4.64 (s, 2H), 3.85 (s, 3H), 2.08 (s, 3H), 1.05 (s, 9H).

### (5-(hydroxymethyl)-1-methyl-1H-pyrazol-3-yl)methyl acetate

To a stirred solution of (5-(chloromethyl)-1-methyl-1H-pyrazol-3-yl)methyl acetate (2×110 g, 260.66) in THF (1.1 L) were added CsF (78 g, 521.32 mmol), followed by 1M TBAF in THF (104 mL, 104.26 mmol at 0° C., and the mixture was stirred at rt for 4 h. After completion, the mixture was filtered to remove unwanted solid material. The filtrate was concentrated to give a crude reside that was purified by silica gel column chromatography eluting with EtOAc afforded to (5-(hydroxymethyl)-1-methyl-1H-pyrazol-3-yl)methyl acetate (62 g) as a pale yellow solid. MS (LCMS) m/z 185.07 [M+H]⁺. ¹H NMR (400 MHz, CDCl₃) δ 6.23 (s, 1H), 5.05 (s, 2H), 4.65 (s, 2H), 3.87 (s, 3H), 2.08 (s, 3H), 1.78 (bs, 1H)

### (5-(chloromethyl)-1-methyl-1H-pyrazol-3-yl)methyl acetate

To a stirred solution of (5-(hydroxymethyl)-1-methyl-1H-pyrazol-3-yl)methyl acetate (16 g, 86.9 mmol) in DCM (160 mL) under Ar was added SOCl₂ (12 mL, 173.9 mmol) at 0° C., and the mixture was stirred at rt for 30 min. The volatiles were evaporated and a crude residue was obtained. The reaction quenched with a sat.NaHCO₃ solution (150 mL) and extracted with DCM (2×150 mL). The combined organic layer was separated, dried over Na₂SO₄, filtered and concentrated to semi pure (5-(chloromethyl)-1-methyl-1H-pyrazol-3-yl)methyl acetate (18 g) as a brown sticky solid that was used without further purification. MS (LCMS) m/z 203.11 [M+H]⁺. ¹H NMR (400 MHz, CDCl₃) δ 6.45 (s, 1H), 5.17 (s, 2H), 4.59 (s, 2H), 4.15 (s, 3H), 2.13 (s, 3H).

### 3-(((3-(hydroxymethyl)-1-methyl-1H-pyrazol-5-yl)methyl)thio)naphthalen-1-ol

To a stirred degassed solution of semi pure (5-(chloromethyl)-1-methyl-1H-pyrazol-3-yl)methyl acetate (18 g, 89.1 mmol) in MeOH (90 mL) and THF (36 mL), were added K₂CO₃ (61.5 g, 445.5 mmol) and intermediate 4 (23.16 g, 89.1 mmol). The mixture was stirred at rt for 4 h. The mixture was filtered, and the filtrate was concentrated to give a crude residue, which was triturated with DCM (50 mL) and n-pentane (50 mL) afford 3-(((3-(hydroxymethyl)-1-methyl-1H-pyrazol-5-yl)methyl)thio)naphthalen-1-ol (12 g, 40.0 mmol) as a pale brown solid. MS (LCMS) m/z 301.07 [M+H]⁺. ¹H NMR (400 MHz, DMSO) δ 8.05-8.03, (d, J=8.0 Hz, 1H), 7.31-7.29, (d, J=8.0 Hz, 1H), 7.11-7.07, (t, J=8.0 Hz, 1H), 6.92-6.88, (t, J=8.0 Hz, 1H), 6.30 (s, 1H), 6.12 (s, 1H), 5.95 (s, 1H), 4.28 (s, 2H), 3.74 (s, 3H), 3.19 (s, 2H).

### (5-(((4-hydroxynaphthalen-2-yl)thio)methyl)-1-methyl-1H-pyrazol-3-yl)methyl acetate

To a stirred solution of 3-(((3-(hydroxymethyl)-1-methyl-1H-pyrazol-5-yl)methyl)thio)naphthalen-1-ol (12 g, 40.13 mmol) in AcOH (120 mL) was added KF (4.6 g, 80.26 mmol), and the mixture was stirred at 100° C. for 48 h. The mixture was then concentrated to give a crude residue, which was diluted with EtOAc (500 mL) and washed with a sat. NaHCO₃ solution (500 mL). The organic layer was separated, dried over Na₂SO₄, filtered and evaporated to give a semi pure compound that was purified by silica gel column chromatography using 50% EtOAc in PE to afford (5-(((4-(allyloxy)naphthalen-2-yl)thio)methyl)-1-methyl-1H-pyrazol-3-yl)methyl acetate (6.6 g, 9.47 mmol, 40% over 3 steps) as a pale yellow liquid. MS (LCMS) m/z 301.07 [M+H]⁺. ¹H NMR (400 MHz, CDCl₃) δ 8.19-8.17, (d, J=8.0 Hz, 1H), 7.72-7.70, (d, J=8.0 Hz, 1H), 7.51-7.44, (m, 3H), 7.17, (bs, 1H), 6.50 (s, 1H), 6.09 (s, 1H), 5.03 (s, 2H), 4.12 (s, 2H), 3.82 (s, 3H), 2.09 (s, 3H).

### (5-(((4-(allyloxy)naphthalen-2-yl)thio)methyl)-1-methyl-1H-pyrazol-3-yl)methyl acetate

To a stirred solution of (5-(((4-(allyloxy)naphthalen-2-yl)thio)methyl)-1-methyl-1H-pyrazol-3-yl)methyl acetate (20.0 g, 58.47 mmol) in MeCN (200 mL) under N₂ atmosphere was added K₂CO₃ (20.17 g, 146.17 mmol) followed by allyl bromide (7.6 mL, 87.71) at 0° C., and the mixture was stirred rt for 16 h. After completion, the mixture was filtered. The filtrate was concentrated to give a crude residue which was diluted with water (100 mL) and extracted with EtOAc (2×100 mL). The organic layer was separated, dried over Na₂SO₄, filtered and evaporated to give a semi pure compound that was purified by silica gel column chromatography (230-400 mesh) using 30 to 50% EtOAc in PE to afford (5-(((4-(allyloxy)naphthalen-2-yl)thio)methyl)-1-methyl-1H-pyrazol-3-yl)methyl acetate (9.2 g) as a yellow color liquid. MS (LCMS) m/z 383.10 [M+H]⁺. ¹H NMR (400 MHz, CDCl3) δ 8.25-8.23, (d, J=8.0 Hz, 1H), 7.70-7.68, (d, J=8.0 Hz, 1H), 7.51-7.44, (m, 2H), 7.39, (s, 1H), 6.65 (s, 1H), 6.19-12 (m, 1H), 6.08 (s, 1H), 5.54-5.49 (m, 1H), 5.37-5.34 (m, 1H), 4.99 (s, 2H), 4.66 (m, 2H), 4.13 (s, 2H), 3.84 (m, 3H), 2.04 (m, 3H).

### (5-(((4-(allyloxy)naphthalen-2-yl)thio)methyl)-1-methyl-1H-pyrazol-3-yl)methanol

To a stirred solution of (5-(((4-(allyloxy)naphthalen-2-yl)thio)methyl)-1-methyl-1H-pyrazol-3-yl)methyl acetate (12.3 g, 31.41 mmol) in MeOH (120 mL), was added K₂CO₃ (8.8 g, 62.82 mmol) at 0° C. The mixture was allowed to stir at 0° C. for 1 h. After completion of starting material, the solvent was evaporated. The mixture was diluted with water (100 mL) and extracted with EtOAc (3×100 mL). The organic layer was separated, dried over Na₂SO₄, filtered and evaporated to give semi pure (5-(((4-(allyloxy)naphthalen-2-yl)thio)methyl)-1-methyl-1H-pyrazol-3-yl)methanol (10.5 g) as a pale yellow liquid. MS (LCMS) m/z 341.29 [M+H]⁺. ¹H NMR (400 MHz, CDCl₃) δ 8.25-8.23, (d, J=8.0 Hz, 1H), 7.69-7.67, (d, J=8.0 Hz, 1H), 7.51-7.44 (m, 2H), 7.38, (s, 1H), 6.67, (s, 1H), 6.19-6.14 (m, 1H), 6.10 (s, 1H), 5.54-5.49 (m, 1H), 5.48-5.37 (m, 1H), 4.66-4.65 (m, 2H), 4.57-4.56 (m, 2H), 4.12 (s, 2H), 3.81 (s, 3H), 2.04 (m, 1H).

### 5-(((4-(allyloxy)naphthalen-2-yl)thio)methyl)-3-(chloromethyl)-1-methyl-1H-pyrazole

To a stirred solution of (5-(((4-(allyloxy)naphthalen-2-yl)thio)methyl)-1-methyl-1H-pyrazol-3-yl)methanol (10.5 g, 30.88 mmol) in DCM (100 mL) under Ar was added SOCl₂ (2.46 mL, 33.97 mmol) at 0° C., and the mixture was stirred at same temperature for 1 h. The volatiles were evaporated to give semi pure 5-(((4-(allyloxy)naphthalen-2-yl)thio)methyl)-3-(chloromethyl)-1-methyl-1H-pyrazole (10.5 g) as a brown sticky solid that was used without further purification. MS (LCMS) m/z 359.06 [M+H]⁺. ¹H NMR (400 MHz, CDCl3) δ 8.27-8.25, (m, 1H), 7.71-7.69, (m, 1H), 7.55-7.48 (m, 2H), 7.39, (s, 1H), 6.69, (s, 1H), 6.19 (s, 1H), 6.18-6.10 (m, 1H), 5.54-5.49 (m, 1H), 5.39-5.36 (m, 1H), 4.69-4.67 (m, 2H), 4.58 (s, 2H), 4.10 (s, 2H), 3.97 (s, 3H).

### S-((5-(((4-(allyloxy)naphthalen-2-yl)thio)methyl)-1-methyl-1H-pyrazol-3-yl)methyl) ethanethioate (15)

To a stirred solution of 5-(((4-(allyloxy)naphthalen-2-yl)thio)methyl)-3-(chloromethyl)-1-methyl-1H-pyrazole (10.5 g, 29.32 mmol) dissolved in DMF (140 mL) was added KSAc (5.7 g, 49.86 mmol) at 0° C., and the mixture was allowed to stir at rt for 16 h. The mixture was diluted with ice cold water (150 mL) and extracted with EtOAc (2×150 mL). The organic layer was separated, dried over Na₂SO₄, filtered and evaporated to give a semi pure compound that was purified by Grace normal phase eluting with 35% EtOAc in PE afford S-((5-(((4-(allyloxy)naphthalen-2-yl)thio)methyl)-1-methyl-1H-pyrazol-3-yl)methyl) ethanethioate (15, 5.6 g) as a pale yellow gummy liquid. MS (LCMS) m/z 399.53 [M+H]⁺. ¹H NMR (400 MHz, CDCl3) δ 8.25-8.23, (m, 1H), 7.70-7.68, (m, 1H), 7.51-7.46 (m, 2H), 7.38, (s, 1H), 6.52, (s, 1H), 6.21-6.11 (m, 1H), 5.97 (m, 1H), 5.55-5.50 (m, 1H), 5.37-5.34 (m, 1H), 4.66-4.64 (m, 2H), 4.13-4.11 (s, 2H), 4.09-4.07 (s, 2H), 3.78 (s, 3H), 2.29 (s, 3H).

### Intermediate 16

### ((3-(ethoxycarbonyl)-1-methyl-1H-pyrazol-5-yl)methyl)triphenylphosphonium chloride (16)

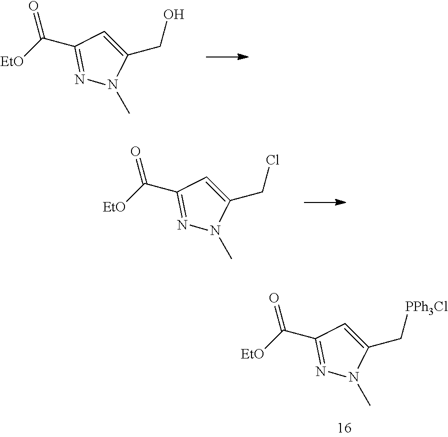

### Ethyl 5-(chloromethyl)-1-methyl-1H-pyrazole-3-carboxylate

To a stirred solution of ethyl 5-(hydroxymethyl)-1-methyl-1H-pyrazole-3-carboxylate (2×15 g, 81.52 mmol) in DCM (150 mL) under Ar was added SOCl₂ (10.67 g, 89.67 mmol) at 0° C., and the mixture was stirred rt for 60 min. The volatiles were evaporated and a crude residue was obtained. The mixture was basified with a sat. NaHCO₃ solution (150 mL) and extracted with DCM (2×150 mL). The combined organic layers were dried over Na₂SO₄, filtered and concentrated to afford ethyl 5-(chloromethyl)-1-methyl-1H-pyrazole-3-carboxylate as a brown sticky oil that was used without further purification. MS (LCMS) m/z 203.02 [M+H]⁺.

### ((3-(ethoxycarbonyl)-1-methyl-1H-pyrazol-5-yl) methyl)triphenylphosphonium chloride (16)

To the solution of ethyl 5-(chloromethyl)-1-methyl-1H-pyrazole-3-carboxylate (2) (2×15 g, 74.25 mmol) in acetonitrile (150 mL) was added triphenylphosphine (21.4 g, 81.68 mmol). The mixture was stirred at 85° C. for 16 h. The solvent was removed under reduced pressure. The residue was triturated with EtOAc (500 mL), and the resulting solids were collected by filtration and washed with EtOAc (200 mL) to afford ((3-(ethoxycarbonyl)-1-methyl-1H-pyrazol-5-yl)methyl)triphenylphosphonium chloride (16, 60 g, 80% over 2-steps) as a white solid. ¹HNMR (300 MHz, CD₃OD) δ 1.29 (t, J=6.9 Hz, 3H), 3.32 (s, 2H), 4.28 (q, J=7.2 Hz, 2H), 4.84 (s, 3H), 6.39 (s, 1H), 7.70-7.82 (m, 12H), 7.95-7.99 (m, 3H).

### Intermediate 17

### 3-(2-(3-(aminomethyl)-1-methyl-1H-pyrazol-5-yl)ethyl)naphthalen-1-ol (17)

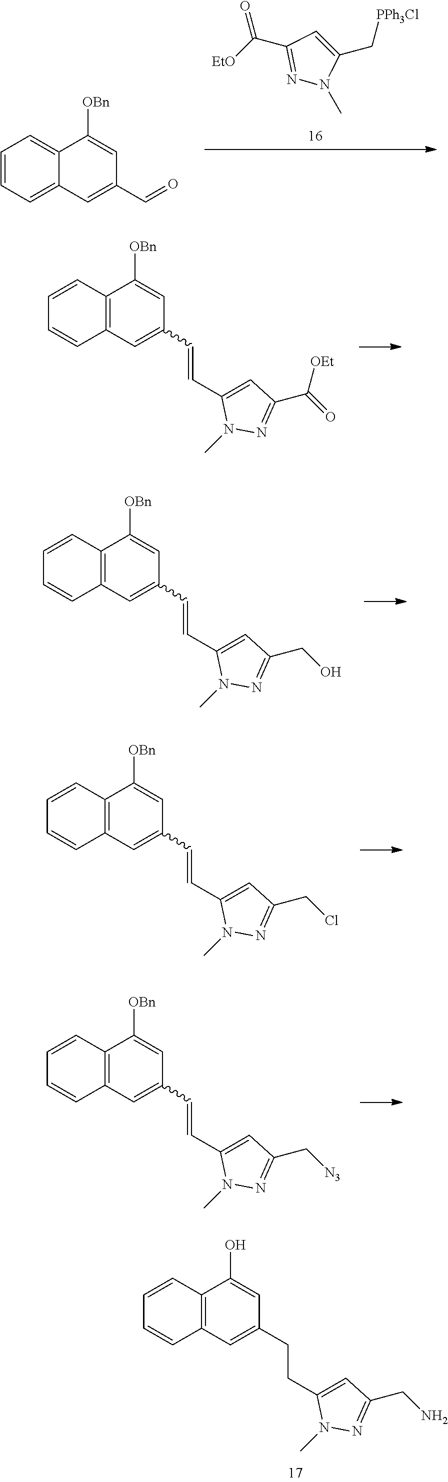

### (E/Z) Ethyl 5-(2-(4-(benzyloxy)naphthalen-2-yl)vinyl)-1-methyl-1H-pyrazole-3-carboxylate

To the suspension of ((3-(ethoxycarbonyl)-1-methyl-1H-pyrazol-5-yl) methyl) triphenylphosphonium chloride (16) (2×21.2 g, 45.80 mmol) in anhydrous THF (100 mL) was added NaH (2×2.2 g, 91.60 mmol) (60% in oil) at 0° C. under N₂ atmosphere. After stirring at 0° C. for 40 min, the mixture was cooled to −20° C. 4-(benzyloxy)-2-naphthaldehyde (2×8 g, 30.53 mmol) in THF (50 mL) was added at −20° C. slowly. The mixture was stirred at −10° C. for 2 h. The reaction was quenched with a sat. NH₄Cl solution (500 mL) at −10° C. The aq. phase was extracted with EtOAc (2×500 mL). The combined organic layers mixed, dried over Na₂SO₄, filtered and concentrated to dryness. The crude material was purified by silica gel column chromatography. The compound was eluted using 20% ethyl acetate in PE to afford (E/Z) ethyl 5-(2-(4-(benzyloxy)naphthalen-2-yl)vinyl)-1-methyl-1H-pyrazole-3-carboxylate (20 g, 72%, 48.54 mmol) as an off white semi solid. MS (LCMS) m/z 413.26 [M+H]⁺.

### (E/Z) (5-(2-(4-(benzyloxy)naphthalen-2-yl)vinyl)-1-methyl-1H-pyrazol-3-yl)methanol

A solution of (E/Z) ethyl 5-(2-(4-(benzyloxy)naphthalen-2-yl)vinyl)-1-methyl-1H-pyrazole-3-carboxylate (2×10 g, 24.21 mmol) in anhydrous THF (150 mL) was cooled to 0° C. LAH (10 mL, 24.21 mmol) (2.4 M in THF) was added at 0° C. After stirring at 0° C. for 30 min and then rt for 90 min, the reaction was quenched with sat. Na₂SO₄ (5 mL) and 15% NaOH (5 mL) at 0° C. The mixture was stirred at 0° C. for 15 min and RT for 30 min. The suspension was filtered, and the solid cake was washed with EtOAc (2×700 mL). The combined organic layers were mixed and concentrated to dryness. The crude was purified by silica gel column chromatography. The compound was eluted using 5% methanol in DCM to afford (E/Z) (5-(2-(4-(benzyloxy)naphthalen-2-yl)vinyl)-1-methyl-1H-pyrazol-3-yl)methanol (16 g, 88%, 43 mmol) as an off white solid. MS (LCMS) m/z 371.45 [M+H]⁺.

### (E/Z) 5-(2-(4-(benzyloxy)naphthalen-2-yl)vinyl)-3-(chloromethyl)-1-methyl-1H-pyrazole

A mixture of (E/Z) (5-(2-(4-(benzyloxy)naphthalen-2-yl)vinyl)-1-methyl-1H-pyrazol-3-yl)methanol (2×12.5 g, 33.78 mmol) was dissolved in DCM (125 mL) and cooled to 0° C. Thionyl chloride (4.42 g, 37.16 mmol) was added at 0° C. After addition, the mixture was stirred at rt for 2 h. The mixture was cooled to 0° C., diluted with DCM (500 mL) and quenched with sat. NaHCO₃ (300 mL). The layers were separated. The organic phase was washed with sat. NaHCO₃ (2×30 mL) and brine (1×300 mL). The organic layers were dried over Na₂SO₄, filtered and concentrated to dryness to afford crude of (E/Z) 5-(2-(4-(benzyloxy)naphthalen-2-yl)vinyl)-3-(chloromethyl)-1-methyl-1H-pyrazole (24 g) as a brown oil. MS (LCMS) m/z 389.21 [M+H]⁺.

### (E/Z) 3-(azidomethyl)-5-(2-(4-(benzyloxy)naphthalen-2-yl)vinyl)-1-methyl-1H-pyrazole

To the solution of (E/Z) 5-(2-(4-(benzyloxy)naphthalen-2-yl)vinyl)-3-(chloromethyl)-1-methyl-1H-pyrazole (2×12 g, 30.92 mmol) in acetonitrile (120 mL) were added NaI (4.6 g, 30.92 mmol) and sodium azide (10 g, 154 mmol). After stirring at 85° C. for 3 h, the mixture was cooled to rt and then diluted with water (500 mL). The aq. phase was extracted with EtOAc (2×500 mL). The combined organic layers were mixed, dried over Na₂SO₄, filtered and concentrated to dryness. The crude was purified by silica gel column chromatography. The compound was eluted using 20% ethyl acetate in PE to afford (E/Z) 3-(azidomethyl)-5-(2-(4-(benzyloxy)naphthalen-2-yl)vinyl)-1-methyl-1H-pyrazole (16 g, 61%) as an off white solid. MS (LCMS) m/z 396.51 [M+H]⁺.

### 3-(2-(3-(aminomethyl)-1-methyl-1H-pyrazol-5-yl)ethyl)naphthalen-1-ol (17)

To the degassed solution of (E/Z) 3-(azidomethyl)-5-(2-(4-(benzyloxy)naphthalen-2-yl)vinyl)-1-methyl-1H-pyrazole (2×5 g, 12.62 mmol) in EtOAc (100 mL) and MeOH (100 mL) was added Pd/C (2 g) (10 wt % on C). The mixture was stirred at rt in parr shaker under H₂ pressure (70 psi) for 24 h. The mixture was filtered through a pad of Celite that was washed with MeOH (3×200 mL). The combined filtrate were mixed and concentrated to dryness. The resulting solid was triturated with diethyl ether (2×100 mL) to afford 3-(2-(3-(aminomethyl)-1-methyl-1H-pyrazol-5-yl)ethyl)naphthalen-1-ol (17, 1. 73 g, 48%, 6.12 mmol) as an off white solid. MS (ES+) m/z 282.42 [M+H]⁺. ¹H NMR (300 MHz, DMSO-d₆) δ 2.93 (br s, 4H), 3.55-3.63 (m, 5H), 6.02 (s, 1H), 6.78 (s, 1H), 7.21 (s, 1H), 7.33-7.46 (m, 2H), 7.73 (d, J=7.8 Hz, 1H), 8.03 (d, J=7.8 Hz, 1H).

### Example 1

### (Z)-1⁶-Chloro-2⁵-cyano-1¹,2¹,6¹-trimethyl-1¹H,2¹H,6¹H-10-oxa-4,8-dithia-1(7,3)-indola-2(4,3),6(3,5)-dipyrazola-9(3,1)-naphthalenacyclotridecaphane-1²-carboxylic acid

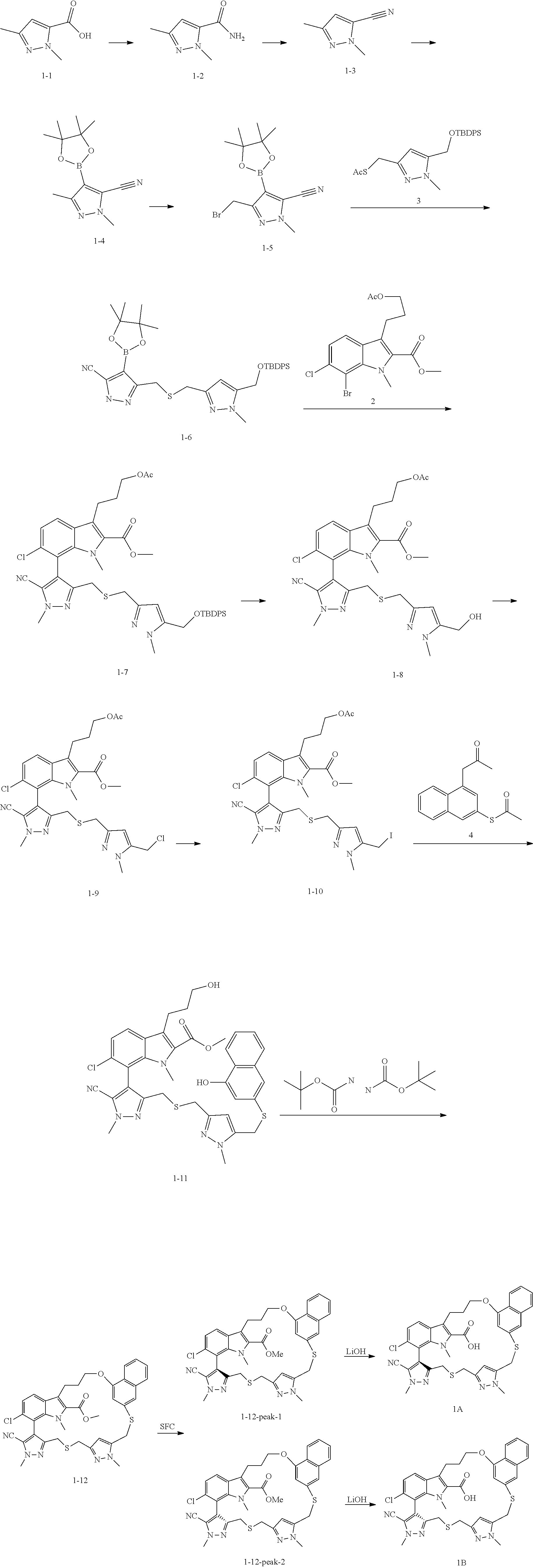

To a stirred solution of 1,3-dimethyl-1H-pyrazole-5-carboxylic acid (1-1) (100 g, 714.28 mmol) in DCM (1 L) was added EDC-HCl (250 g, 191.70 mmol), DMAP (174 g, 1.428 mmol, 2 eq.) and NH₄Cl (75 g, 1.428 mmol, 2 eq.), at 0° C., and the mixture was stirred at rt for 16 h. The mixture was diluted with water (1 L) and extracted with 10% MeOH in DCM (6×500 mL). The organic layer was separated, dried over Na₂SO₄, filtered and evaporated to afford semi-pure compound (1-2). The solid compound was stirred in (500 mL) of DCM, and the mixture filtered to afford 1,3-dimethyl-1H-pyrazole-5-carboxamide (1-2) (68 g, 489.20 mmol, 69%) as a white colour solid. This compound was used without further purification. MS (LCMS) m/z 140.32 [M+H]⁺.

To a stirred solution of compound (1-2) (77 g, 553.95 mmol) in pyridine (1 L) was added POCl₃ (71 mL) at 0° C., and the mixture was stirred at rt for 4 h. The reaction was quenched with 6N HCl (500 mL) and extracted with EtOAc (3×500 mL). The combined organic layer was washed with a sat. NaHCO₃ solution (2×300 mL) and brine (200 mL), dried over Na₂SO₄, filtered and evaporated to afford 1,3-dimethyl-1H-pyrazole-5-carbonitrile (1-3) (26 g, 214.87 mmol, 63%) as an off-white solid. This compound was used without further purification. MS (LCMS) m/z 122.25 [M+H]⁺.

To a stirred solution of compound (1-3) (2.5 g, 20.66 mmol) in hexane (50 mL) were added bis(pinacolato dibaron) (6.29 g, 24.79 mmol) 4,4′-bis(1,1-dimethylethyl)-2,2′-bipyridine (330 mg, 1.239 mmol) and [Ir(OMe)COD]₂ (410 mg, 0.619 mmol). The solution was degassed with Ar for 5 min. The mixture was heated at 80° C. for 12 h. The hexane was evaporated to afford semi-pure compound (1-4). The compound was purified by reverse phase GRACE purification using 50% ACN in water to afford 1,3-dimethyl-4-(4,4,5,5-tetramethyl-1,3,2-dioxaborolan-2-yl)-1H-pyrazole-5-carbonitrile (1-4) (2.58 g, 10.44 mmol, 40%) as an off white solid. MS (LCMS) m/z 248.19 [M+H]⁺.

To a stirred solution of compound (1-4) (9 g, 36.43 mmol) in CCl₄ (90 mL) were added NBS (7.7 g, 43.72 mmol) and AIBN (597 mg, 3.643 mmol), and the mixture stirred at 80° C. for 16 h. The mixture was diluted with water (300 mL) and extracted with DCM (3×200 mL). The organic layer was separated, dried over Na₂SO₄, filtered and evaporated to afford semi-pure compound (1-5). The compound was purified by silica gel column chromatography to afford 3-(bromomethyl)-1-methyl-4-(4,4,5,5-tetramethyl-1,3,2-dioxaborolan-2-yl)-1H-pyrazole-5-carbonitrile (1-5) (7.7 g, 23.69 mmol, 27%) as a light yellow liquid. MS (LCMS) m/z 328.09 [M+H]⁺.

To a stirred solution of compound (1-5) (7.7 g, 23.69 mmol) in MeOH (77 mL), THF (15 mL) were added K₂CO₃ (3.59 g, 26.061 mmol), and the mixture was degassed with Ar for 10 min. In another round-bottom flask, S-((5-(((tert-butyldiphenylsilyl)oxy)methyl)-1-methyl-1H-pyrazol-3-yl)methyl) ethanethioate (3) (10.38 g, 23.692 mmol) in methanol (15 mL) was degassed with Ar for 10 min, and this solution was added to previous mixture dropwise. The mixture was stirred at rt for 16 h. The solvent was evaporated, and the mixture was diluted with water (200 mL) and extracted with EtOAc (3×200 mL). The organic layer was separated, dried over Na₂SO₄, filtered and evaporated to afford semi-pure 1-6. The compound was purified by silica gel column chromatography using 40% EtOAc in PE to afford 3-((((5-(((tert-butyldiphenylsilyl)oxy)methyl)-1-methyl-1H-pyrazol-3-yl)methyl)thio)methyl)-1-methyl-4-(4,4,5,5-tetramethyl-1,3,2-dioxaborolan-2-yl)-1H-pyrazole-5-carbonitrile (1-6) (1.75 g, 2.73 mmol, 11%) as a dark brown semi-solid. MS (LCMS) m/z 642.48 [M+H]⁺.

To a stirred solution of compound (1-6) (750 mg, 1.17 mmol) in 1,4-dioxane (10 mL) were added methyl 3-(3-acetoxypropyl)-7-bromo-6-chloro-1-methyl-1H-indole-2-carboxylate (2) (469 mg, 1.17 mmol) and Cs₂CO₃ (1.14 g, 3.51 mmol). The solution was degassed with Ar and dichloro[1,1′-bis(di-tert-butylphosphino)ferrocene]palladium(II) (152 mg, 0.234 mmol) was added. The solution was degassed for 5 min. The mixture was heated at 100° C. for 3 h. 1,4-dioxane was evaporated. The mixture was passed through a Celite pad and washed with EtOAc (200 mL). The organic layer was washed with water (100 mL), separated, dried over Na₂SO₄, filtered and evaporated to afford semi-pure compound (1-7). The compound was purified by silica gel column chromatography using 60% EtOAc in PE to afford methyl 3-(3-acetoxypropyl)-7-(3-((((5-(((tert-butyldiphenylsilyl)oxy)methyl)-1-methyl-1H-pyrazol-3-yl)methyl)thio)methyl)-5-cyano-1-methyl-1H-pyrazol-4-yl)-6-chloro-1-methyl-1H-indole-2-carboxylate (1-7) (425 mg, 0.508 mmol, 43%) as a brown semi solid. MS (LCMS) m/z 837.50 [M+H]⁺.

To a stirred solution of compound (1-7) (1.7 g, 2.033 mmol) in THF (17 mL) was added TBAF (1 M in THF, 4.06 mL, 4.06 mmol) at 0° C., and the mixture stirred at rt for 4 h. The reaction was quenched with water (50 mL) and extracted with EtOAc (2×100 mL). The organic layer was washed with water (50 mL) and brine (50 mL). The organic layer was separated, dried over Na₂SO₄, filtered and evaporated to afford semi-pure compound (1-8). The compound was purified by silica gel column chromatography using 70% EtOAc in PE to afford methyl 3-(3-acetoxypropyl)-6-chloro-7-(5-cyano-3-((((5-(hydroxymethyl)-1-methyl-1H-pyrazol-3-yl)methyl)thio)methyl)-1-methyl-1H-pyrazol-4-yl)-1-methyl-1H-indole-2-carboxylate (1-8) (900 mg, 1.50 mmol, 74%) as a pale yellow semi solid. MS (LCMS) m/z 599.37 [M+H]⁺.

To a stirred solution of compound (1-8) (400 mg, 0.668 mmol) in DCM (4 mL) under Ar was added SOCl₂ (0.06 mL, 0.802 mmol) at 0° C., and the mixture was stirred at rt for 2 h. The mixture was diluted with DCM (15 mL) and washed with a sat. NaHCO₃ solution (3×10 mL). The organic layer was separated, dried over Na₂SO₄, filtered and evaporated to afford semi-pure methyl 3-(3-acetoxypropyl)-6-chloro-7-(3-((((5-(chloromethyl)-1-methyl-1H-pyrazol-3-yl)methyl)thio)methyl)-5-cyano-1-methyl-1H-pyrazol-4-yl)-1-methyl-1H-indole-2-carboxylate (1-9) (400 mg, 0.649 mmol) as a brown semi solid. This compound was used without further purification. MS (LCMS) m/z 617.31 [M+H]⁺.

To a stirred solution of semi-pure compound (1-9) (400 mg, 0.649 mmol) in dry MeCN (4 mL) were added NaI (175 mg, 1.168 mmol) at rt, and the mixture was heated at 80° C. for 2 h. The solvent was evaporated, and the mixture was diluted with water (15 mL) and extracted with EtOAc (3×20 mL). The organic layer was separated, dried over Na₂SO₄, filtered and evaporated to afford semi-pure methyl 3-(3-acetoxypropyl)-6-chloro-7-(5-cyano-3-((((5-(iodomethyl)-1-methyl-1H-pyrazol-3-yl)methyl)thio)methyl)-1-methyl-1H-pyrazol-4-yl)-1-methyl-1H-indole-2-carboxylate (1-10) (400 mg, 0.564 mmol) as a brown semi solid. This compound was used without further purification. MS (LCMS) m/z 709.29 [M+H]⁺.

To a stirred solution of semi-pure compound (1-10) (400 mg, 0.564 mmol) in MeOH (2 mL), THF (0.1 mL) were added K₂CO₃ (390 mg, 2.824 mmol) and degassed with Ar for 10 min. In another round bottom flask, 3-(acetylthio)naphthalen-1-yl acetate (4) (161 mg, 0.621 mmol) in MeOH (2 mL) was degassed with Ar for 10 min, and this solution was added to previous mixture dropwise. The mixture was stirred at rt for 1 h. The solvent was evaporated, and the mixture was diluted with water (10 mL) and extracted with EtOAc (3×10 mL). The organic layer was separated, dried over Na₂SO₄, filtered and evaporated to afford semi-pure compound (1-11). The compound was purified by silica gel column chromatography using 70% EtOAc in PE to afford methyl 6-chloro-7-(5-cyano-3-((((5-(((4-hydroxynaphthalen-2-yl)thio)methyl)-1-methyl-1H-pyrazol-3-yl)methyl)thio)methyl)-1-methyl-1H-pyrazol-4-yl)-3-(3-hydroxypropyl)-1-methyl-1H-indole-2-carboxylate (1-11) (160 mg, 0.224 mmol, 33% for three steps) as a light brown solid. MS (LCMS) m/z 715.37 [M+H]⁺.

To a stirred solution of TPP (59 mg, 0.224 mmol) in toluene (1 mL) was added a solution of compound (1-11) (2×80 mg, 0.112 mmol) in toluene (1 mL) and THF (0.1 mL), and the mixture was stirred at rt for 16 h. The reaction was quenched with water (5 mL) and extracted with EtOAc (2×10 mL). The organic layer was dried over Na₂SO₄, filtered and concentrated to give semi-pure compound (1-12). The compound was purified by silica gel column chromatography using 50% EtOAc in PE to afford methyl (Z)-1⁶-chloro-2⁵-cyano-1¹,2¹,6¹-trimethyl-1¹H,2¹H,6¹H-10-oxa-4,8-dithia-1(7,3)-indola-2 (4,3),6(3,5)-dipyrazola-9(3,1)-naphthalenacyclotridecaphane-12-carboxylate (1-12) (70 mg, 0.100 mmol, 44%) as an off-white sticky solid. The compound was purified by SFC to get pure isomers as peak-1 and peak-2 shown on the next step. MS (LCMS) m/z 697.37 [M+H]⁺.

Altogether, compound (70 mg, 1-12) was purified by SFC purification to afford (1-13-Peak-1) (21 mg) as an off-white solid with 99% of LCMS purity (chiral HPLC:99%) and (1-14-Peak-2) (6 mg) as an off-white solid with 99% of LCMS purity (chiral HPLC: 99%). The two peaks were separately used for the next steps to afford the respective final compounds. (1-13-peak-1): MS (LCMS) m/z 697.44 [M+H]⁺. ¹H NMR (400 MHz, CDCl₃) δ 8.33-8.30 (m, 1H), 7.74-7.72 (m, 1H), 7.62 (d, J=8.8 Hz, 1H), 7.57-7.50 (m, 3H), 6.97 (d, J=8.4 Hz, 1H), 6.19 (d, J=1.2 Hz, 1H), 4.94 (s, 1H), 4.11 (s, 3H), 3.90-3.82 (m, 5H), 3.80-3.71 (m, 1H), 3.70-3.65 (m, 6H), 3.62-3.49 (m, 3H), 3.31-3.22 (m, 2H), 3.04 (d, J=14.0 Hz, 1H), 2.63 (d, J=14.0 Hz, 1H), 2.49-2.11 (m, 2H). (1-14-peak-2): MS (LCMS) m/z 697.41 [M+H]⁺. ¹H NMR (400 MHz, CDCl₃) δ 8.33-8.30 (m, 1H), 7.74-7.72 (m, 1H), 7.62 (d, J=8.8 Hz, 1H), 7.57-7.50 (m, 3H), 6.97 (d, J=8.4 Hz, 1H), 6.20 (s, 1H), 4.94 (s, 1H), 4.11 (s, 3H), 3.90-3.82 (m, 5H), 3.80-3.71 (m, 1H), 3.70-3.65 (m, 6H), 3.62-3.49 (m, 3H), 3.31-3.22 (m, 2H), 3.04 (d, J=13.6 Hz, 1H), 2.63 (d, J=14.0 Hz, 1H), 2.49-2.11 (m, 2H). The absolute stereochemistry of compound (1-13-peak-1) and compound (1-14-peak-2) is arbitrarily assigned.

To a stirred solution of compound (1-13-peak-1) (21 mg, 0.0301 mmol) in MeOH/THF (1:1, 1 mL) was added a solution of LiOH.H₂O (6.3 mg, 0.150 mmol) and water (0.5 mL) at 0° C., and the mixture was stirred at rt for 4 h. The solvent was evaporated. The aqueous layer was acidified to pH 2 using 6 N aq. HCl and extracted with 10% MeOH in DCM. The organic layer was separated, dried over Na₂SO₄, filtered and evaporated to afford semi-pure (1A). The compound was purified by silica gel Prep-TLC using 5% MeOH in DCM to afford (Z)-1⁶-chloro-2⁵-cyano-1¹,2¹,6¹-trimethyl-1¹H,2¹H,6¹H-10-oxa-4,8-dithia-1 (7,3)-indola-2 (4,3),6(3,5)-dipyrazola-9 (3,1)-naphthalenacyclotridecaphane-12-carboxylic acid (1A) (8 mg, 0.0117 mmol, 38%) as an off-white solid. MS (LCMS) m/z 683.24 [M+H]⁺. ¹H NMR (400 MHz, DMSO-d₆) δ 8.08 (d, J=8.0 Hz, 1H), 7.89 (d, J=8.4 Hz, 1H), 7.71 (d, J=8.0 Hz, 1H), 7.50-7.42 (m, 2H), 7.36 (s, 1H), 7.15 (d, J=8.8 Hz, 1H), 6.71 (s, 1H), 4.89 (s, 1H), 4.45-4.21 (m, 2H), 4.12-4.05 (m, 4H), 3.95-3.85 (m, 1H), 3.70 (s, 3H), 3.60-3.45 (m, 4H), 3.39-3.23 (m, 2H), 3.15-3.92 (m, 3H), 2.32-2.15 (m, 2H).

To a stirred solution of compound (1-14-peak-2) (30 mg, 0.043 mmol) in MeOH/THF (1:1, 1 mL) was added a solution of LiOH.H₂O (9 mg, 0.215 mmol) and water (0.5 mL) at 0° C., and the mixture was stirred at rt for 4 h. The solvent was evaporated. The aqueous layer was acidified to pH 2 using 6 N aq. HCl and extracted with 10% MeOH in DCM. The organic layer was separated, dried over Na₂SO₄, filtered and evaporated to afford semi-pure (1B). The compound was purified by silica gel Prep-TLC to afford (Z)-1⁶-chloro-2⁵-cyano-1¹,2¹,6¹-trimethyl-1¹H,2¹H,6¹H-10-oxa-4,8-dithia-1 (7,3)-indola-2 (4,3),6(3,5)-dipyrazola-9 (3,1)-naphthalenacyclotridecaphane-12-carboxylic acid (1B) (8.2 mg, 0.0117 mmol, 27%) as an off white solid. MS (LCMS) m/z 683.21 [M+H]⁺. ¹H NMR (400 MHz, DMSO-d₆) δ 8.07 (d, J=8.0 Hz, 1H), 7.89 (d, J=8.4 Hz, 1H), 7.71 (d, J=8.0 Hz, 1H), 7.50-7.42 (m, 2H), 7.35 (s, 1H), 7.13 (d, J=8.4 Hz, 1H), 6.73 (s, 1H), 4.92 (s, 1H), 4.45-4.20 (m, 2H), 4.12-4.05 (m, 4H), 3.95-3.85 (m, 1H), 3.71 (s, 3H), 3.60-3.23 (m, 6H), 3.15-3.93 (m, 3H), 2.32-2.20 (m, 2H).

The absolute stereochemistry of compounds (1A) and (1B) is arbitrarily assigned.

### Example 2

### (Z)-1⁶-Chloro-1¹,6¹-dimethyl-2⁴,2⁵,2⁶,2⁷-tetrahydro-1¹H,6¹H-10-oxa-4,8-dithia-2(3,2)-pyrazolo[1,5-a]pyridina-1(7,3)-indola-6(3,5)-pyrazola-9(3,1)-naphthalenacyclotridecaphane-1²-carboxylic acid

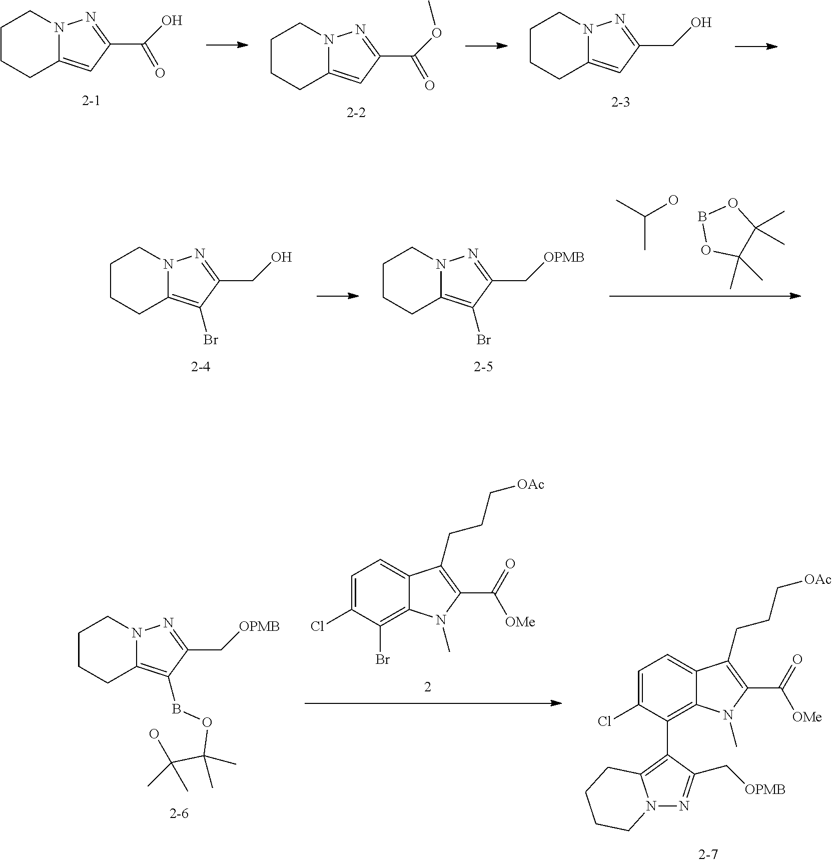

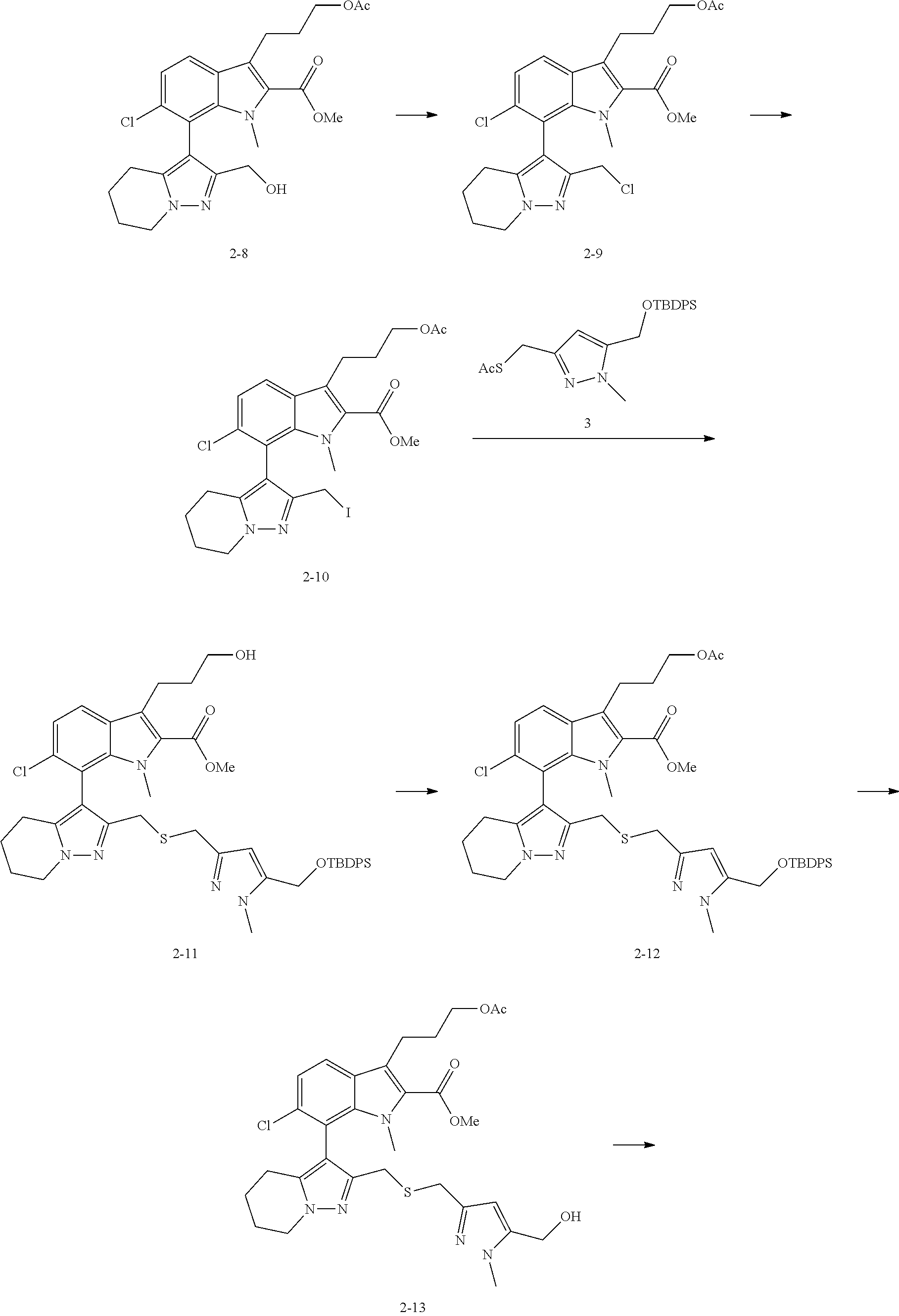

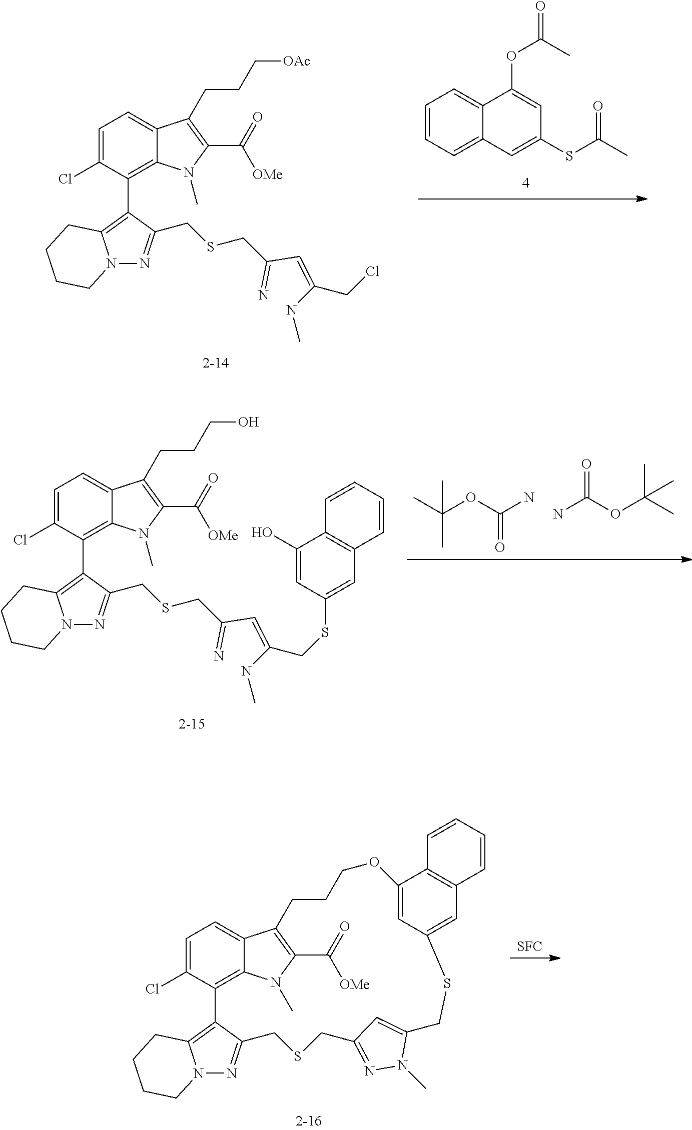

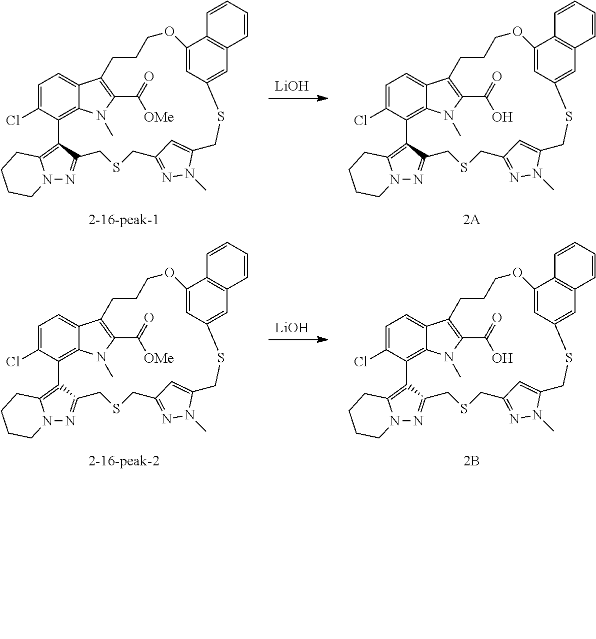

To a stirred solution of 4,5,6,7-tetrahydropyrazolo[1,5-a]pyridine-2-carboxylic acid (2-1) (5 g, 30.08 mmol) in MeOH (50 mL) was added SOCl₂ (7.16 g, 60.18 mmol) at 0° C., and the mixture was stirred at reflux temperature for 6 h. The solvent was evaporated and co-distilled with MeOH and dried under high vacuum to afford semi-pure methyl 4,5,6,7-tetrahydropyrazolo[1,5-a]pyridine-2-carboxylate (2-2) (5.5 g, 30.52 mmol) as an off-white solid. This compound was used without further purification. MS (LCMS) m/z 181.13 [M+H]⁺.

To a stirred solution of semi-pure compound (2-2) (5.5 g, 30.52 mmol) in THF (100 mL) was added LiAlH₄ (2M in THF, 30.5 mL, 61.04 mmol) at 0° C., and the mixture was stirred at rt for 2 h. The reaction was quenched with ice cooled a sat. NH₄Cl solution (50 mL) and extracted EtOAc (4×100 mL). The organic layer was separated, dried over Na₂SO₄, filtered and evaporated to afford semi-pure (4,5,6,7-tetrahydropyrazolo[1,5-a]pyridin-2-yl)methanol (2-3) (3.5 g, 23 mmol, 76%) as a brown viscous liquid. This compound was used without further purification. MS (LCMS) m/z 153.13 [M+H]⁺.

To a stirred solution of compound (2-3) (3.9 g, 25.62 mmol) in DCM (40 mL) was added NBS (4.56 g, 25.62 mmol) at 0° C., and the mixture was stirred at rt for 2 h. The mixture was diluted with a sat. NaHCO₃ solution (50 mL) and extracted with DCM (3×50 mL). The organic layer was separated, dried over Na₂SO₄, filtered and evaporated to afford semi-pure compound (2-4). The compound was triturated with pentane:ether (1:1) (3×20 mL) to afford semi-pure (3-bromo-4,5,6,7-tetrahydropyrazolo[1,5-a]pyridin-2-yl)methanol (2-4) (5.5 g, 23.80 mmol, 92%) as a brown viscous liquid. MS (LCMS) m/z 231.01 [M+H]⁺.

To a stirred solution of semi-pure compound (2-4) (5.5 g, 23.80 mmol) in DMF (55 mL) was added NaH (60% in oil, 857 mg, 35.7 mmol) at 0° C., and the mixture was stirred at rt for 20 min. 1-(chloromethyl)-4-methoxybenzene (5.22 g, 33.32 mmol) and KI (395 mg, 2.38 mmol) were then added, and the mixture was stirred at rt for 16 h. The reaction was quenched with a sat. aq. NH₄Cl solution (60 mL). The mixture was extracted with EtOAc (4×75 mL). The combined organic layer was washed with water (2×75 mL) and brine (75 mL), dried over Na₂SO₄ and concentrated under reduced pressure to afford semi-pure compound (2-5). The compound was purified by silica gel column chromatography eluting with 10-20% EtOAc/PE to afford 3-bromo-2-(((4-methoxybenzyl)oxy)methyl)-5,6-dihydro-4H-pyrrolo[1,2-b]pyrazole (2-5) (5.1 g, 14.52 mmol, 61%) as an off-white solid. MS (LCMS) m/z 351 [M+H]⁺.

To a stirred solution of compound (2-5) (5 g, 14.23 mmol) in THF (100 mL) was added n-BuLi (1.6 M in hexanes, 17.8 mL, 28.47 mmol) at −78° C., and the mixture was stirred at −78° C. for 50 min. 2-isopropoxy-4,4,5,5-tetramethyl-1,3,2-dioxaborolane (5.30 g, 28.47 mmol) was added at −78° C. The mixture temperature was slowly raised to rt and stirred for 3 h. The solvents were evaporated. The mixture was diluted with EtOAc (75 mL), filtered through a Celite pad, and the filtrate was evaporated to afford semi-pure 2-(((4-methoxybenzyl)oxy)methyl)-3-(4,4,5,5-tetramethyl-1,3,2-dioxaborolan-2-yl)-4,5,6,7-tetrahydropyrazolo[1,5-a]pyridine (2-6) (7 g crude) as a colourless liquid. This compound was used without further purification. MS (LCMS) m/z 399.39 [M+H]⁺.

To a stirred solution of semi-pure compound (2-6) (16×1 g, 2.51 mmol) in DMF (10 mL) were added methyl 3-(3-acetoxypropyl)-7-bromo-6-chloro-1-methyl-1H-indole-2-carboxylate (2) (1.01 g, 1.76 mmol) and Cs₂CO₃ (1.23 g, 3.77 mmol). The solution was degassed with Ar and Pd(PPh₃)₄(145 mg, 0.126 mmol) was added. The mixture was degassed for 5 min, and then heated at 115° C. for 48 h. The mixture was diluted with EtOAc (100 mL), passed through a Celite pad and washed with EtOAc (200 mL). The organic layer was washed with water (3×100 mL), separated, dried over Na₂SO₄, filtered and evaporated to afford semi-pure compound (2-7). The compound was purified by neutral alumina column chromatography using 10% MeOH in DCM to afford methyl 3-(3-acetoxypropyl)-6-chloro-7-(2-(((4-methoxybenzyl)oxy)methyl)-4,5,6,7-tetrahydropyrazolo[1,5-a]pyridin-3-yl)-1-methyl-1H-indole-2-carboxylate (2-7) (1.7 g, 2.86 mmol, 15% yield for two steps) as a brown semi-solid. MS (LCMS) m/z 594.44 [M+H]⁺.

To a stirred solution of compound (2-7) (2×800 mg, 1.35 mmol) in DCM (10 mL) was added TFA (1.54 g, 13.46 mmol) at 0° C., and the mixture was stirred at rt for 1.5 h. The reaction was quenched with a sat. aq. NaHCO₃ solution (25 mL) and extracted with DCM (3×25 mL). The organic layer was washed with water (50 mL) and brine (50 mL), separated, dried over Na₂SO₄, filtered and evaporated to afford semi-pure compound (2-8). The compound was purified by silica gel column chromatography using 10% MeOH in DCM to afford methyl 3-(3-acetoxypropyl)-6-chloro-7-(2-(hydroxymethyl)-4,5,6,7-tetrahydropyrazolo[1,5-a]pyridin-3-yl)-1-methyl-1H-indole-2-carboxylate (2-8) (742 mg, 2.74 mmol, 58%) as a brown semi-solid. MS (LCMS) m/z 474.37 [M+H]⁺.

To a stirred solution of compound (2-8) (2×750 mg, 1.58 mmol) in DCM (10 mL) under Ar was added SOCl₂ (226 mg, 1.90 mmol) at 0° C., and the mixture was stirred at rt for 1 h. The mixture was diluted with DCM (20 mL) and washed with a sat. NaHCO₃ solution (3×10 mL). The organic layer was separated, dried over Na₂SO₄, filtered and evaporated to afford semi-pure methyl 3-(3-acetoxypropyl)-6-chloro-7-(2-(chloromethyl)-4,5,6,7-tetrahydropyrazolo[1,5-a]pyridin-3-yl)-1-methyl-1H-indole-2-carboxylate (2-9) (1.5 g, 3.05 mmol) as a brown semi-solid. This compound was used without further purification. MS (LCMS) m/z 492.38 [M+H]⁺.

To a stirred solution of semi-pure compound (2-9) (2×750 mg, 0.95 mmol) in dry MeCN (10 mL) were added NaI (256 mg, 1.70 mmol) at rt, and the mixture was heated to 80° C. for 2 h. The solvent was evaporated. The mixture was diluted with water (20 mL) and extracted with EtOAc (3×30 mL). The organic layer was separated, dried over Na₂SO₄, filtered and evaporated to afford semi-pure methyl 3-(3-acetoxypropyl)-6-chloro-7-(2-(iodomethyl)-4,5,6,7-tetrahydropyrazolo[1,5-a]pyridin-3-yl)-1-methyl-1H-indole-2-carboxylate (2-10) (1.6 g, 2.74 mmol) as a brown semi-solid. This compound was used without further purification. MS (LCMS) m/z 584.21 [M+H]⁺.

To a stirred solution of semi-pure compound (2-10) (2×800 mg, 1.37 mmol) in MeOH (10 mL) and THF (5 mL) were added K₂CO₃ (947 mg, 6.85 mmol). The mixture was degassed with Ar for 10 min. In another round bottom flask, S-((5-(((tert-butyldiphenylsilyl)oxy)methyl)-1-methyl-1H-pyrazol-3-yl)methyl) ethanethioate (3) (600 mg, 1.37 mmol) in MeOH (5 mL) was degassed with Ar for 10 min. This solution was added to previous mixture dropwise. The mixture was stirred at rt for 16 h. The solvent was evaporated. The mixture was diluted with water (20 mL) and extracted with EtOAc (3×30 mL). The organic layer was separated, dried over Na₂SO₄, filtered and evaporated to afford semi-pure compound (2-11). The compound was purified by silica gel column chromatography using 50-70% EtOAc in PE to afford methyl 7-(2-((((5-(((tert-butyldiphenylsilyl)oxy)methyl)-1-methyl-1H-pyrazol-3-yl)methyl)thio)methyl)-4,5,6,7-tetrahydropyrazolo[1,5-a]pyridin-3-yl)-6-chloro-3-(3-hydroxypropyl)-1-methyl-1H-indole-2-carboxylate (2-11) (1.5 g, 1.85 mmol, 58% for three steps) as a light brown liquid. MS (LCMS) m/z 810.32 [M+H]⁺.

To a stirred solution of compound (2-11) (1.5 g, 1.85 mmol) in DCM (15 mL) were added Et₃N (374 mg, 3.7 mmol), DMAP (5 mg, cat.) and Ac₂O (227 mg, 2.22 mmol) at 0° C., and the mixture was stirred at rt for 2 h. The mixture was diluted with DCM (20 mL), and washed with water (2×20 mL) and brine (20 mL). The organic layer was separated, dried over Na₂SO₄, filtered and evaporated to afford semi-pure compound (2-12). The compound was purified by silica gel column chromatography using 30-50% EtOAc/PE to afford methyl 3-(3-acetoxypropyl)-7-(2-((((5-(((tert-butyldiphenylsilyl)oxy)methyl)-1-methyl-1H-pyrazol-3-yl)methyl)thio)methyl)-4,5,6,7-tetrahydropyrazolo[1,5-a]pyridin-3-yl)-6-chloro-1-methyl-1H-indole-2-carboxylate (2-12) (1.2 g, 1.12 mmol, 71%) as a brown semi solid. MS (LCMS) m/z 852.68 [M+H]⁺.

To a stirred solution of compound (2-12) (1.1 g, 1.29 mmol) in THF (10 mL) was added TBAF (1 M in THF, 2.6 mL, 2.6 mmol) at 0° C., and the mixture was stirred at rt for 2 h. The reaction was quenched with water (20 mL) and extracted with EtOAc (3×25 mL). The organic layer was washed with water (25 mL) and brine (25 mL), separated, dried over Na₂SO₄, filtered and evaporated to afford semi-pure compound (2-13). The compound was purified by silica gel column chromatography using 70% EtOAc in PE to afford methyl 3-(3-acetoxypropyl)-6-chloro-7-(2-((((5-(hydroxymethyl)-1-methyl-1H-pyrazol-3-yl)methyl)thio)methyl)-4,5,6,7-tetrahydropyrazolo[1,5-a]pyridin-3-yl)-1-methyl-1H-indole-2-carboxylate (2-13) (600 mg, 0.97 mmol, 75%) as a brown semi solid. MS (LCMS) m/z 614.64 [M+H]⁺.

To a stirred solution of compound (2-13) (2×300 mg, 0.49 mmol) in DCM (6 mL) under Ar was added SOCl₂ (70 mg, 0.59 mmol) at 0° C., and the mixture was stirred at rt for 1 h. The mixture was diluted with DCM (15 mL) and washed with a sat. NaHCO₃ solution (3×10 mL). The organic layer was separated, dried over Na₂SO₄, filtered and evaporated to afford semi-pure methyl 3-(3-acetoxypropyl)-6-chloro-7-(2-((((5-(chloromethyl)-1-methyl-1H-pyrazol-3-yl)methyl)thio)methyl)-4,5,6,7-tetrahydropyrazolo[1,5-a]pyridin-3-yl)-1-methyl-1H-indole-2-carboxylate (2-14) (600 mg, 0.95 mmol) as a brown semi solid. The compound was used without further purification. MS (LCMS) m/z 632.53 [M+H]⁺.

To a stirred solution of semi-pure compound (2-14) (600 mg, 0.95 mmol) (2×300 mg, 0.47 mmol) in MeOH (2 mL) and THF (0.1 mL) were added K₂CO₃ (328 mg, 2.37 mmol). The mixture was degassed with Ar for 10 min. In another round bottom flask, 3-(acetylthio)naphthalen-1-yl acetate (4) (123 mg, 0.47 mmol) in MeOH (2 mL) was degassed with Ar for 10 min. This solution was added to previous mixture dropwise. The mixture was stirred at rt for 1 h. The solvent was evaporated. The mixture was diluted with water (10 mL) and extracted with EtOAc (3×10 mL). The organic layer was separated, dried over Na₂SO₄, filtered and evaporated to afford semi-pure compound (2-15). The compound was purified by silica gel column chromatography using 70% EtOAc in pet-ether to afford methyl 6-chloro-7-(2-((((5-(((4-hydroxynaphthalen-2-yl)thio)methyl)-1-methyl-1H-pyrazol-3-yl)methyl)thio)methyl)-4,5,6,7-tetrahydropyrazolo[1,5-a]pyridin-3-yl)-3-(3-hydroxypropyl)-1-methyl-1H-indole-2-carboxylate (2-15) (140 mg, 0.19 mmol, 20% for two steps) as a light brown solid. MS (LCMS) m/z 730.57 [M+H]⁺.

To a stirred solution of TPP (71 mg, 0.272 mmol) in toluene (0.5 mL) was added a solution of compound (2-15) (100 mg, 0.136 mmol) in toluene (3 mL) and THF (1.2 mL). The mixture was stirred at rt for 16 h. The reaction was quenched with water (5 mL) and extracted with EtOAc (2×10 mL). The organic layer was dried over Na₂SO₄, filtered, concentrated to give semi-pure compound (2-16). The compound was purified by silica gel column chromatography using 50% EtOAc in PE to afford methyl (Z)-1⁶-chloro-1¹,6¹-dimethyl-2⁴,2⁵,2⁶,2⁷-tetrahydro-1¹H,6¹H-10-oxa-4,8-dithia-2(3,2)-pyrazolo[1,5-a]pyridina-1(7,3)-indola-6(3,5)-pyrazola-9(3,1)-naphthalenacyclotridecaphane-1²-carboxylate (2-16) (25 mg, 0.035 mmol, 26%) as an off-white sticky solid. MS (LCMS) m/z 712.16 [M+H]⁺.

Compound (70 mg, 2-16) was purified by SFC purification to afford 2-17-peak-1 (18 mg) as an off-white solid with 93% of LCMS purity (chiral HPLC:99%) and (2-18-peak-2) (15 mg) as an-off white solid with 61% of LCMS purity (chiral HPLC:96%). These two peaks were separately used for the next steps to afford the respective final compounds. The absolute stereochemistry of compound (2-17-peak-1) and compound (2-17-peak-2) is arbitrarily assigned.

(2-17-peak-1): MS (LCMS) m/z 712.16 [M+H]⁺. ¹H NMR (400 MHz, CDCl₃) δ 8.34-8.31 (m, 1H), 7.74-7.71 (m, 1H), 7.55-7.49 (m, 4H), 6.92 (d, J=8.4 Hz, 1H), 6.26 (d, J=1.2 Hz, 1H), 4.88 (s, 1H), 4.21-4.15 (m, 2H), 3.92-3.75 (m, 4H), 3.75-3.60 (m, 9H), 3.52-3.44 (m, 2H), 3.30-3.05 (m, 3H), 2.68-2.40 (m, 4H), 2.25-1.95 (m, 3H), 1.90-1.75 (m, 2H). (2-18-peak-2): MS (LCMS) m/z 712.16 [M+H]⁺. ¹H NMR (400 MHz, CDCl₃) δ 8.34-8.31 (m, 1H), 7.74-7.71 (m, 1H), 7.55-7.49 (m, 4H), 6.92 (d, J=8.4 Hz, 1H), 6.26 (d, J=1.2 Hz, 1H), 4.88 (s, 1H), 4.21-4.15 (m, 2H), 3.92-3.75 (m, 4H), 3.75-3.60 (m, 9H), 3.52-3.44 (m, 2H), 3.30-3.05 (m, 3H), 2.68-2.40 (m, 4H), 2.25-1.95 (m, 3H), 1.90-1.75 (m, 2H).

To a stirred solution of compound (2-17-peak-1) (18 mg, 0.025 mmol) in MeOH/THF/H₂O (7:7:3, 1 mL) was added a solution of LiOH.H₂O (5.5 mg, 0.126 mmol) at 0° C. The mixture stirred at rt for 4 h, and the solvent was evaporated. The aqueous layer was acidified to pH 2 using 6 N aqueous HCl, extracted with 10% MeOH in DCM. The organic layer was separated, dried over Na₂SO₄, filtered and evaporated to afford semi-pure compound, which was triturated with pentane and ether to afford (Z)-1⁶-chloro-1¹,6¹-dimethyl-2⁴,2⁵,2⁶,2⁷-tetrahydro-1¹H,6¹H-10-oxa-4,8-dithia-2(3,2)-pyrazolo[1,5-a]pyridina-1(7,3)-indola-6(3,5)-pyrazola-9(3,1)-naphthalenacyclotridecaphane-1²-carboxylic acid (2A) (12 mg, 0.017 mmol, 68%) as an off-white solid. MS (LCMS) m/z 698.16 [M+H]⁺. ¹H NMR (400 MHz, DMSO-d₆) δ 13.30 (br s, 1H), 8.09 (d, J=7.6 Hz, 1H), 7.86 (d, J=8.8 Hz, 1H), 7.72 (d, J=7.2 Hz, 1H), 7.51-7.41 (m, 2H), 7.38 (s, 1H), 7.13 (d, J=8.8 Hz, 1H), 6.67 (s, 1H), 4.74 (s, 1H), 4.26 (s, 2H), 4.16-4.02 (m, 3H), 3.90-3.81 (m, 1H), 3.71 (s, 3H), 3.52 (s, 3H), 3.48-3.40 (m, 2H), 3.22-2.87 (m, 4H), 2.40-2.15 (m, 4H), 2.01-1.90 (m, 2H), 1.80-1.68 (m, 2H).

To a stirred solution of compound (2-18-peak-2) (15 mg, 0.021 mmol) in MeOH/THF/H₂O (7:7:3, 1 mL) was added a solution of LiOH.H₂O (5.5 mg, 0.1 mmol) at 0° C. The mixture stirred at rt for 4 h, and the solvent was evaporated. The aqueous layer was acidified to pH 2 using 6 N aqueous HCl, extracted with 10% MeOH in DCM. The organic layer was separated, dried over Na₂SO₄, filtered and evaporated to afford semi-pure compound, which was further purified by silica gel prep TLC running with 30% EtOAc/PE to afford (Z)-1⁶-chloro-1¹,6¹-dimethyl-2⁴,2⁵,2⁶,2⁷-tetrahydro-1¹H,6¹H-10-oxa-4,8-dithia-2(3,2)-pyrazolo[1,5-a]pyridina-1(7,3)-indola-6(3,5)-pyrazola-9(3,1)-naphthalenacyclotridecaphane-1²-carboxylic acid (2B) (6 mg, 0.008 mmol, 38%) as an off-white solid. MS (LCMS) m/z 698.16 [M+H]⁺. ¹H NMR (400 MHz, DMSO-d₆) δ 8.06 (d, J=7.6 Hz, 1H), 7.68 (d, J=7.6 Hz, 1H), 7.62 (d, J=8.0 Hz, 1H), 7.50-7.40 (m, 2H), 7.32 (s, 1H), 7.02 (d, J=8.4 Hz, 1H), 6.79 (s, 1H), 5.01 (s, 1H), 4.55-4.15 (m, 2H), 4.12-4.01 (m, 3H), 3.99-3.89 (m, 1H), 3.73 (s, 3H), 3.51 (s, 3H), 3.48-3.40 (m, 2H), 3.22-2.80 (m, 4H), 2.40-2.15 (m, 4H), 2.01-1.90 (m, 2H), 1.80-1.69 (m, 2H).

The absolute stereochemistry of compounds (2A) and (2B) is arbitrarily assigned.

### Example 3

### (Z)-1⁶-Chloro-1¹,6¹-dimethyl-2⁵,2⁶-dihydro-1¹H,2⁴H,6¹H-10-oxa-4,8-dithia-1(7,3)-indola-2(3,2)-pyrrolo[1,2-b]pyrazola-6(3,5)-pyrazola-9(3,1)-naphthalenacyclotridecaphane-1²-carboxylic acid

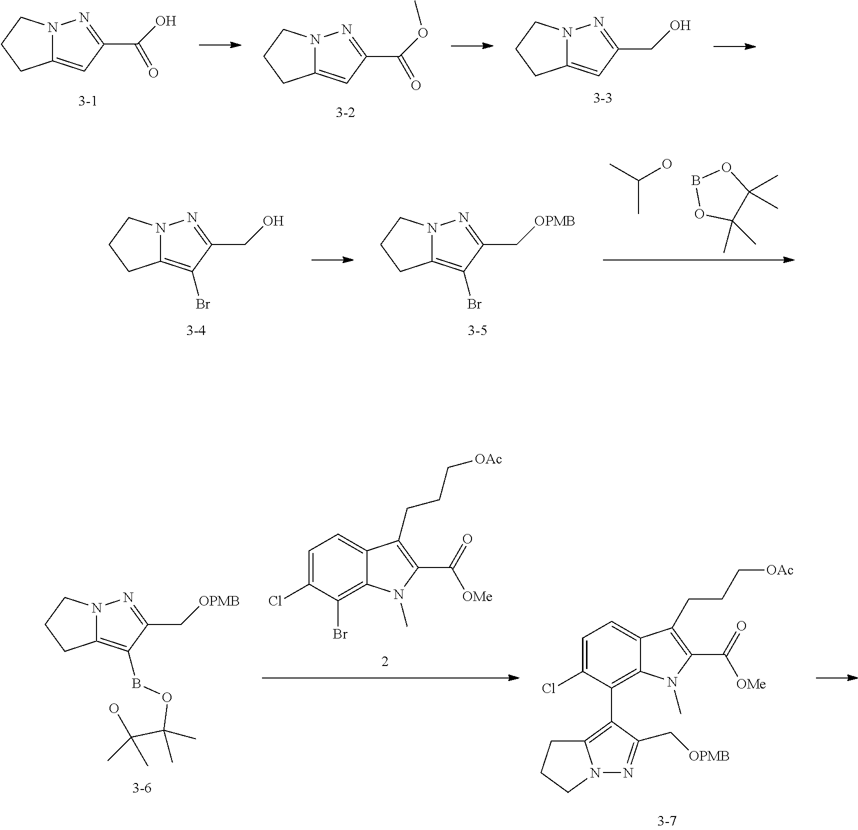

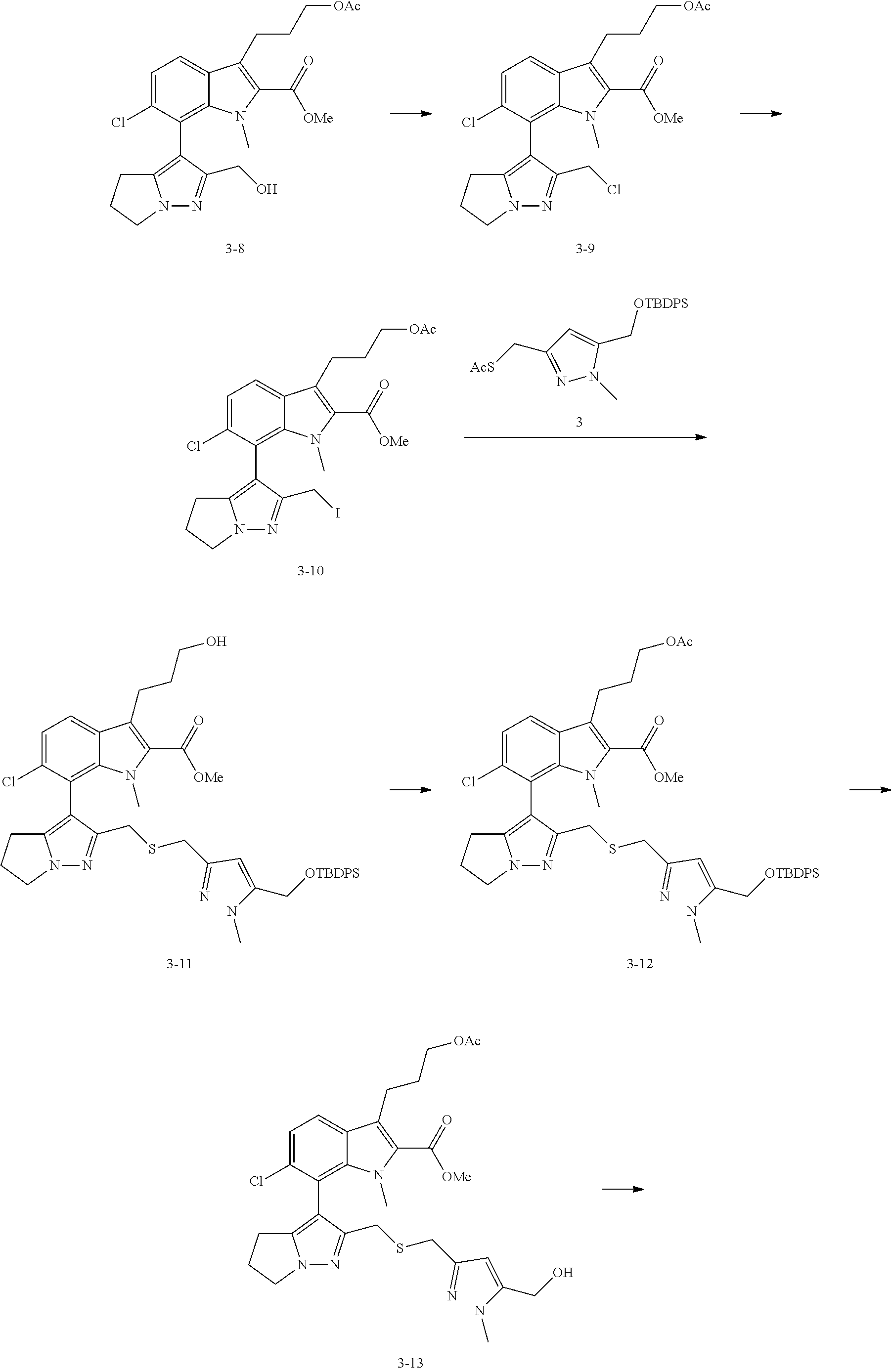

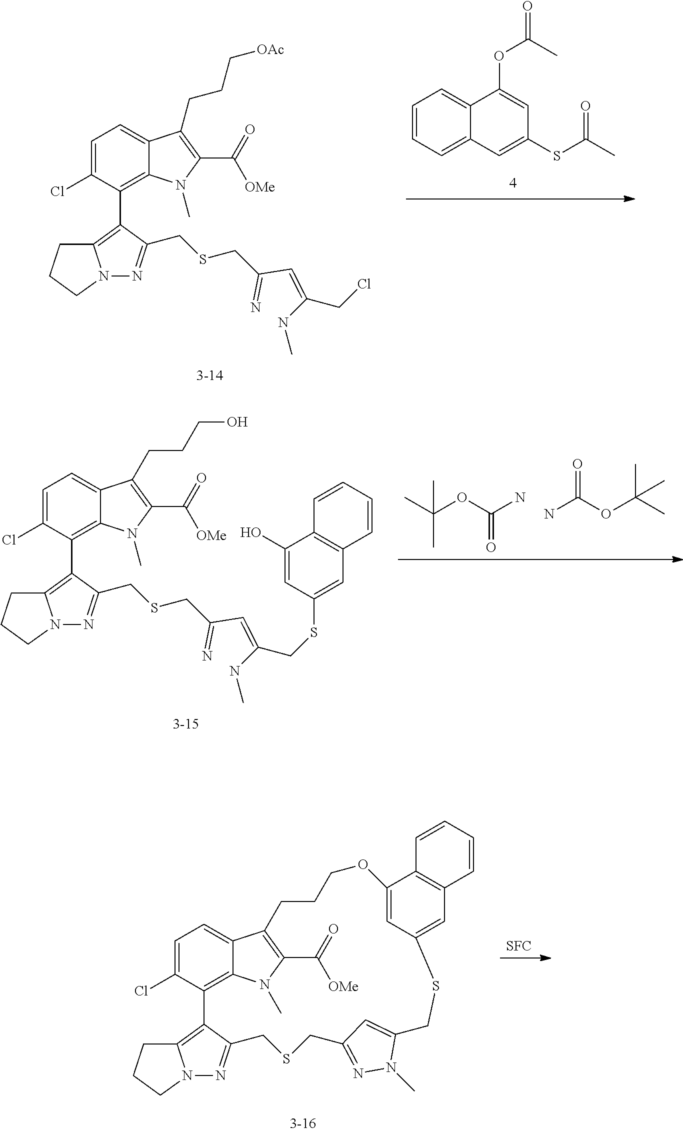

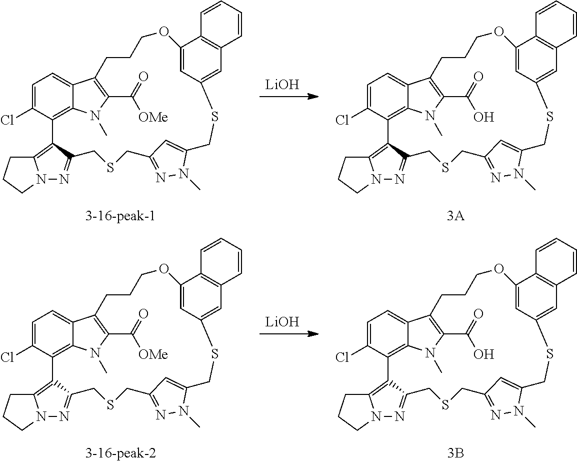

To a stirred solution of 5,6-dihydro-4H-pyrrolo[1,2-b]pyrazole-2-carboxylic acid (3-1) (10 g, 65.72 mmol) in MeOH (100 mL) was added SOCl₂ (15.64 g, 131.45 mmol) at 0° C., and the mixture was stirred at reflux temperature for 6 h. The solvent was evaporated and co-distilled with MeOH and dried under high vac to afford methyl 5,6-dihydro-4H-pyrrolo[1,2-b]pyrazole-2-carboxylate (3-2) (10 g, 60.18 mmol, 92%) as an off-white solid. The compound was used without further purification. MS (LCMS) m/z 167.09 [M+H]⁺.

To a stirred solution of compound (3-2) (10 g, 60.18 mmol) in THF (100 mL) was added LiAlH₄ (2M in THF, 60.2 mL, 120.4 mmol) at 0° C., and the mixture was stirred at rt for 2 h. The reaction was quenched with ice cooled a sat. NH₄Cl solution (100 mL) and extracted EtOAc (4×200 mL). The organic layer was separated, dried over Na₂SO₄, filtered and evaporated to afford semi-pure compound (3-3). The compound was purified by silica gel column chromatography using 20% EtOAc in PE to afford (5,6-dihydro-4H-pyrrolo[1,2-b]pyrazol-2-yl)methanol (3-3) (6.5 g, 47.04 mmol, 78%) as an off-white solid. MS (LCMS) m/z 139.09 [M+H]⁺.

To a stirred solution of compound (3-3) (4 g, 28.95 mmol) in DCM (50 mL) was added NBS (5.18 g, 28.95 mmol) at 0° C., and the mixture was stirred at rt for 2 h. The mixture was diluted with a sat. NaHCO₃ solution (50 mL) and extracted with DCM (3×50 mL). The organic layer was separated, dried over Na₂SO₄, filtered and evaporated to afford semi-pure compound (3-4). The compound was triturated with pentane-ether (1:1) (3×20 mL) to afford (3-bromo-5,6-dihydro-4H-pyrrolo[1,2-b]pyrazol-2-yl)methanol (3-4) (5.0 g, 23.03 mmol, 79%) as a light yellow solid. MS (LCMS) m/z 217.01 [M+H]⁺.

To a stirred solution of compound (4-4) (2×4 g, 18.42 mmol) in DMF (40 mL) was added NaH (60% in oil, 1.1 g, 27.64 mmol) at 0° C., and the mixture was stirred at rt for 20 min. 1-(Chloromethyl)-4-methoxybenzene (4.04 g, 25.79 mmol) and KI (300 mg, 1.81 mmol) were added, and the mixture was stirred at rt for 16 h. After completion of reaction, reaction was quenched with a sat. aq. NH₄Cl solution (50 mL). The mixture was extracted with EtOAc (4×50 mL). The combined organic layer was washed with water (2×50 mL) and brine (50 mL), dried over Na₂SO₄ and concentrated under reduced pressure to afford semi-pure compound (3-5). The compound was purified by silica gel column chromatography eluting with 10-20% EtOAc/PE to afford 3-bromo-2-(((4-methoxybenzyl)oxy)methyl)-5,6-dihydro-4H-pyrrolo[1,2-b]pyrazole (3-5) (6.8 g, 20.16 mmol, 54% yield for two batches) as a light yellow solid. MS (LCMS) m/z 336.96 [M+H]⁺.

To a stirred solution of compound (3-5) (2×4 g, 11.86 mmol) in THF (110 mL) was added n-BuLi (1.6 M in hexanes, 14.8 mL, 23.72 mmol) at −78° C., and the mixture was stirred at −78° C. for 50 min. 2-isopropoxy-4,4,5,5-tetramethyl-1,3,2-dioxaborolane (3.09 g, 16.60 mmol) was added at −78° C. The mixture temperature was slowly raised to rt and stirred for 3 h. The solvents were evaporated. The mixture was diluted with EtOAc (75 mL), filtered through a Celite pad, and the filtrate was evaporated to afford semi-pure 2-(((4-methoxybenzyl)oxy)methyl)-3-(4,4,5,5-tetramethyl-1,3,2-dioxaborolan-2-yl)-5,6-dihydro-4H-pyrrolo[1,2-b]pyrazole (3-6) (10 g for two batches) as a light yellow gummy solid. The compound was used without further purification. MS (LCMS) m/z 385.15 [M+H]⁺.

To a stirred solution of semi-pure compound (3-6) (16×1 g, 2.60 mmol) in 1,4-dioxane (15 mL) were added methyl 3-(3-acetoxypropyl)-7-bromo-6-chloro-1-methyl-1H-indole-2-carboxylate (2) (734 mg, 1.82 mmol) and Cs₂CO₃ (1.69 g, 5.2 mmol). The solution was degassed with Ar. Dichloro[1 1′-bis(di-tert-butylphosphino)ferrocene]palladium(II) (102 mg, 0.156 mmol) was added, and the solution degassed again for 5 min. The mixture was heated at 100° C. for 16 h. 1,4-dioxane was evaporated, and the mixture was passed through a Celite pad and washed with EtOAc (200 mL). The organic layer was washed with water (100 mL). The organic layer was separated, dried over Na₂SO₄, filtered and evaporated to afford semi-pure 3-7. The compound was purified by silica gel column chromatography using 10% MeOH in DCM to afford methyl 3-(3-acetoxypropyl)-7-(3-((((5-(((tert-butyldiphenylsilyl)oxy)methyl)-1-methyl-1H-pyrazol-3-yl)methyl)thio)methyl)-5-cyano-1-methyl-1H-pyrazol-4-yl)-6-chloro-1-methyl-1H-indole-2-carboxylate (3-7) (3.5 g, 6.03 mmol, 16%) as a brown liquid. MS (LCMS) m/z 580.36 [M+H]⁺.

To a stirred solution of compound (3-7) (2×2.0 g, 3.45 mmol) in DCM (20 mL) was added TFA (3.94 g, 34.5 mmol) at 0° C., and the mixture was stirred at rt for 1.5 h. The reaction was quenched with a sat. aq. NaHCO₃ solution (25 mL), extracted with DCM (3×25 mL). The organic layer was washed with water (50 mL) and brine (50 mL). The organic layer was separated, dried over Na₂SO₄, filtered and evaporated to afford semi-pure compound (3-8). The compound was purified by silica gel column chromatography using 10% MeOH in DCM to afford methyl 3-(3-acetoxypropyl)-6-chloro-7-(2-(hydroxymethyl)-5,6-dihydro-4H-pyrrolo[1,2-b]pyrazol-3-yl)-1-methyl-1H-indole-2-carboxylate (3-8) (2.3 g, 5.03 mmol, 73%) as a brown sticky liquid. MS (LCMS) m/z 460.29 [M+H]⁺.

To a stirred solution of compound (3-8) (2×750 mg, 1.63 mmol) in DCM (10 mL) under Ar was added SOCl₂ (233 mg, 1.96 mmol) at 0° C., and the mixture was stirred at rt for 1 h. The mixture was diluted with DCM (20 mL) and washed with a sat. NaHCO₃ solution (3×10 mL). The organic layer was separated, dried over Na₂SO₄, filtered and evaporated to afford semi-pure methyl 3-(3-acetoxypropyl)-6-chloro-7-(2-(chloromethyl)-5,6-dihydro-4H-pyrrolo[1,2-b]pyrazol-3-yl)-1-methyl-1H-indole-2-carboxylate (3-9) (1.6 g, 3.34 mmol) as a brown sticky solid. The compound was used without further purification. MS (LCMS) m/z 478.26 [M+H]⁺.

To a stirred solution of semi-pure compound (3-9) (2×800 mg, 1.67 mmol) in dry MeCN (10 mL) were added NaI (452 mg, 3.01 mmol) at rt, and the mixture was heated to 80° C. for 2 h. The solvent was evaporated. The mixture was diluted with water (20 mL) and extracted with EtOAc (3×30 mL). The organic layer was separated, dried over Na₂SO₄, filtered and evaporated to afford semi-pure methyl 6-chloro-7-(3-(iodomethyl)-1,5-dimethyl-1H-pyrazol-4-yl)-3-(3-methoxy-3-oxopropyl)-1-methyl-1H-indole-2-carboxylate (3-10) (1.8 g, 3.16 mmol) as a brown sticky solid. The compound was used without further purification. MS (LCMS) m/z 570.27 [M+H]⁺.

To a stirred solution of semi-pure compound (3-10) (2×900 mg, 1.58 mmol) in MeOH (10 mL) and THF (5 mL) were added K₂CO₃ (1.09 g, 7.9 mmol). The solution was degassed with Ar for 10 min. In another round bottom flask, S-((5-(((tert-butyldiphenylsilyl)oxy)methyl)-1-methyl-1H-pyrazol-3-yl)methyl) ethanethioate (3) (832 mg, 1.90 mmol) in MeOH (5 mL) was degassed with Ar for 10 min. This solution was added to previous mixture dropwise. The mixture stirred at rt for 16 h. The solvent was evaporated, and the mixture was diluted with water (20 mL) and extracted with EtOAc (3×30 mL). The organic layer was separated, dried over Na₂SO₄, filtered and evaporated to afford semi-pure 3-11. The compound was purified by silica gel column chromatography using 50-70% EtOAc in PE to afford methyl 7-(2-((((5-(((tert-butyldiphenylsilyl)oxy)methyl)-1-methyl-1H-pyrazol-3-yl)methyl)thio)methyl)-5,6-dihydro-4H-pyrrolo[1,2-b]pyrazol-3-yl)-6-chloro-3-(3-hydroxypropyl)-1-methyl-1H-indole-2-carboxylate (3-11) (900 mg, 1.13 mmol, 35% for three steps) as brown sticky liquid. MS (LCMS) m/z 796.55 [M+H]⁺.

To a stirred solution of compound (3-11) (2×750 mg, 0.94 mmol) in DCM (10 mL) were added Et₃N (190 mg, 1.88 mmol), DMAP (5 mg, cat.) and Ac₂O (116 mg, 1.13 mmol) at 0° C. The mixture was stirred at rt for 2 h. The mixture was then diluted with DCM (20 mL), and washed with water (2×20 mL) and brine (20 mL). The organic layer was separated, dried over Na₂SO₄, filtered and evaporated to afford semi-pure compound (3-12). The compound was purified by silica gel column chromatography using 30-50% EtOAc:PE to afford methyl 3-(3-acetoxypropyl)-7-(2-((((5-(((tert-butyldiphenylsilyl)oxy)methyl)-1-methyl-1H-pyrazol-3-yl)methyl)thio)methyl)-5,6-dihydro-4H-pyrrolo[1,2-b]pyrazol-3-yl)-6-chloro-1-methyl-1H-indole-2-carboxylate (3-12) (940 mg, 1.12 mmol, 59%) as a brown sticky solid. MS (LCMS) m/z 838.58 [M+H]⁺.

To a stirred solution of compound (3-12) (840 mg, 1 mmol) in THF (10 mL) was added TBAF (1 M in THF, 2 mL, 2 mmol) at 0° C., and the mixture stirred at rt for 2 h. The reaction was quenched with water (20 mL) and extracted with EtOAc (3×25 mL). The organic layer was washed with water (25 mL) and brine (25 mL). The organic layer was then separated, dried over Na₂SO₄, filtered and evaporated to afford semi-pure 3-13. The compound was purified by silica gel column chromatography using 70% EtOAc in PE to afford methyl 3-(3-acetoxypropyl)-6-chloro-7-(2-((((5-(hydroxymethyl)-1-methyl-1H-pyrazol-3-yl)methyl)thio)methyl)-5,6-dihydro-4H-pyrrolo[1,2-b]pyrazol-3-yl)-1-methyl-1H-indole-2-carboxylate (3-13) (580 mg, 0.96 mmol, 96%) as a brown sticky solid. MS (LCMS) m/z 600.38 [M+H]⁺.

To a stirred solution of compound (3-13) (600 mg, 1 mmol) in DCM (6 mL) under Ar was added SOCl₂ (143 mg, 1.2 mmol) at 0° C., and the mixture was stirred at rt for 1 h. The mixture was diluted with DCM (15 mL) and washed with a sat. NaHCO₃ solution (3×10 mL). The organic layer was separated, dried over Na₂SO₄, filtered and evaporated to afford semi-pure methyl 3-(3-acetoxypropyl)-6-chloro-7-(2-((((5-(chloromethyl)-1-methyl-1H-pyrazol-3-yl)methyl)thio)methyl)-5,6-dihydro-4H-pyrrolo[1,2-b]pyrazol-3-yl)-1-methyl-1H-indole-2-carboxylate (3-14) (600 mg, 0.97 mmol) as a brown sticky solid. The compound was used without further purification. MS (LCMS) m/z 618.56 [M+H]⁺.

To a stirred solution of semi-pure compound (3-14) (600 mg, 0.97 mmol) in MeOH (2 mL), THF (0.1 mL) were added K₂CO₃ (402 mg, 2.91 mmol), and the solution degassed with Ar for 10 min. In another round bottom flask, 3-(acetylthio)naphthalen-1-yl acetate (4) (303 mg, 1.16 mmol) in MeOH (2 mL) was degassed with Ar for 10 min. This solution was added to previous mixture dropwise, and the mixture stirred at rt for 1 h. The solvent was evaporated. The mixture was diluted with water (10 mL) and extracted with EtOAc (3×10 mL). The organic layer was separated, dried over Na₂SO₄, filtered and evaporated to afford semi-pure 3-15. The compound was purified by silica gel column chromatography using 70% EtOAc in PE to afford methyl 6-chloro-7-(2-((((5-(((4-hydroxynaphthalen-2-yl)thio)methyl)-1-methyl-1H-pyrazol-3-yl)methyl)thio)methyl)-5,6-dihydro-4H-pyrrolo[1,2-b]pyrazol-3-yl)-3-(3-hydroxypropyl)-1-methyl-1H-indole-2-carboxylate (3-15) (500 mg, 0.70 mmol, 69% for two steps) as a light brown solid. MS (LCMS) m/z 716.52 [M+H]⁺.

To a stirred solution of TPP (257 mg, 0.98 mmol) in toluene (0.5 mL) was added a solution of compound (3-15) (350 mg, 0.49 mmol) in toluene (3 mL) and THF (1.2 mL). The mixture was stirred at rt for 16 h. The reaction was quenched with water (5 mL) and extracted with EtOAc (2×10 mL). The organic layer was dried over Na₂SO₄, filtered, concentrated to give semi-pure 3-16. The compound was purified by silica gel column chromatography using 50% EtOAc in PE to afford methyl (Z)-1⁶-chloro-1¹,6¹-dimethyl-2⁵,2⁶-dihydro-1¹H,2⁴H,6¹H-10-oxa-4,8-dithia-1(7,3)-indola-2(3,2)-pyrrolo[1,2-b]pyrazola-6(3,5)-pyrazola-9(3,1)-naphthalenacyclotridecaphane-1²-carboxylate (3-16) (190 mg, 0.27 mmol, 55%) as an off-white solid. The compound was purified by SFC to afford pure isomers as peak-1 and peak-2 shown on the next step. MS (LCMS) m/z 698.13 [M+H]⁺.

Compound (190 mg, 3-16) was purified by SFC purification to afford (3-17-peak-1) (40 mg) as an off-white solid with 89% of LCMS purity (chiral HPLC:99%) and (3-18-peak-2) (40 mg) as an off-white solid with 95% of LCMS purity (chiral HPLC:98%). These two peaks were separately used for the next steps to afford the respective final compounds. The absolute stereochemistry of compound (3-17-peak-1) and compound (3-18-peak-2) is arbitrarily assigned.

(3-17-peak-1): MS (LCMS) m/z 698.13 [M+H]⁺. ¹H NMR (400 MHz, CDCl₃) δ 8.33-8.30 (m, 1H), 7.74-7.71 (m, 1H), 7.76-7.48 (m, 4H), 6.94 (d, J=8.4 Hz, 1H), 6.25 (d, J=1.2 Hz, 1H), 4.93 (s, 1H), 4.25-4.16 (m, 2H), 3.95-3.75 (m, 6H), 3.74-3.60 (m, 7H), 3.55-3.45 (m, 2H), 3.34-3.03 (m, 3H), 2.80-2.59 (m, 5H), 2.49-2.14 (m, 2H). (3-18-peak-2): MS (LCMS) m/z 698.13 [M+H]⁺. ¹H NMR (400 MHz, CDCl₃) δ 8.33-8.30 (m, 1H), 7.74-7.71 (m, 1H), 7.76-7.48 (m, 4H), 6.44 (d, J=8.8 Hz, 1H), 6.25 (d, J=1.2 Hz, 1H), 4.93 (s, 1H), 4.25-4.16 (m, 2H), 3.95-3.75 (m, 6H), 3.74-3.60 (m, 7H), 3.55-3.45 (m, 2H), 3.34-3.03 (m, 3H), 2.80-2.59 (m, 5H), 2.49-2.14 (m, 2H).

To a stirred solution of compound (3-17-peak-1) (40 mg, 0.06 mmol) in MeOH/THF/H₂O (7:7:3, 1 mL) was added a solution of LiOH.H₂O (13 mg, 0.3 mmol) at 0° C., and the mixture was stirred at rt for 16 h. The solvent was evaporated. The aqueous layer was acidified to pH 2 using 6 N aq. HCl, and extracted with 10% MeOH in DCM. The organic layer was separated, dried over Na₂SO₄, filtered and evaporated to afford semi-pure (1F). The compound was triturated with pentane and ether to afford (Z)-1⁶-chloro-1¹,6¹-dimethyl-2⁵,2⁶-dihydro-1¹H,2⁴H,6¹H-10-oxa-4,8-dithia-1(7,3)-indola-2(3,2)-pyrrolo[1,2-b]pyrazola-6(3,5)-pyrazola-9(3,1)-naphthalenacyclotridecaphane-1²-carboxylic acid (3A) (33 mg, 0.05 mmol, 83%) as an off-white solid. MS (LCMS) m/z 684.12 [M+H]⁺. ¹H NMR (400 MHz, DMSO-d₆) δ 13.40 (br s, 1H), 8.10 (d, J=7.2 Hz, 1H), 7.84 (d, J=8.4 Hz, 1H), 7.72 (d, J=6.8 Hz, 1H), 7.51-7.43 (m, 2H), 7.38 (s, 1H), 7.11 (d, J=8.4 Hz, 1H), 6.68 (s, 1H), 4.78 (s, 1H), 4.27 (s, 2H), 4.16-4.05 (m, 3H), 3.90-3.81 (m, 1H), 3.71 (s, 3H), 3.54 (s, 3H), 3.48-3.40 (m, 2H), 3.22-2.87 (m, 4H), 2.70-2.55 (m, 4H), 2.40-2.10 (m, 2H).

To a stirred solution of compound (3-18-peak-2) (40 mg, 0.06 mmol) in MeOH/THF/H₂O (7:7:3, 1 mL) was added a solution of LiOH.H₂O (13 mg, 0.3 mmol) at 0° C., and the mixture stirred at rt for 16 h. The solvent was evaporated. The aqueous layer was acidified to pH 2 using 6 N aq. HCl, and extracted with 10% MeOH in DCM. The organic layer was separated, dried over Na₂SO₄, filtered and evaporated to afford semi-pure compound, which was triturated with pentane and ether to afford (Z)-1⁶-chloro-1¹,6¹-dimethyl-2⁵,2⁶-dihydro-1¹H,2⁴H,6¹H-10-oxa-4,8-dithia-1(7,3)-indola-2(3,2)-pyrrolo[1,2-b]pyrazola-6(3,5)-pyrazola-9(3,1)-naphthalenacyclotridecaphane-1²-carboxylic acid (3B) (36 mg, 0.052 mmol, 86%) as an off-white solid. MS (LCMS) m/z 684.12 [M+H]⁺. ¹H NMR (400 MHz, DMSO-d₆) δ 13.40 (br s, 1H), 8.10 (d, J=7.2 Hz, 1H), 7.84 (d, J=8.4 Hz, 1H), 7.72 (d, J=6.8 Hz, 1H), 7.51-7.43 (m, 2H), 7.38 (s, 1H), 7.11 (d, J=8.4 Hz, 1H), 6.67 (s, 1H), 4.77 (s, 1H), 4.26 (s, 2H), 4.18-4.05 (m, 3H), 3.90-3.81 (m, 1H), 3.71 (s, 3H), 3.54 (s, 3H), 3.48-3.40 (m, 2H), 3.22-2.87 (m, 4H), 2.70-2.55 (m, 4H), 2.40-2.12 (m, 2H).

The absolute stereochemistry of compounds (3A) and (3B) is arbitrarily assigned.

### Example 4

### (Z)-1⁶-Chloro-1¹,6¹-dimethyl-2⁶,2⁷-dihydro-1¹H,2⁴H,6¹H-10-oxa-4,8-dithia-2(3,2)-pyrazolo[5,1-c][1,4]oxazina-1(7,3)-indola-6(3,5)-pyrazola-9(3,1)-naphthalenacyclotridecaphane-1²-carboxylic acid

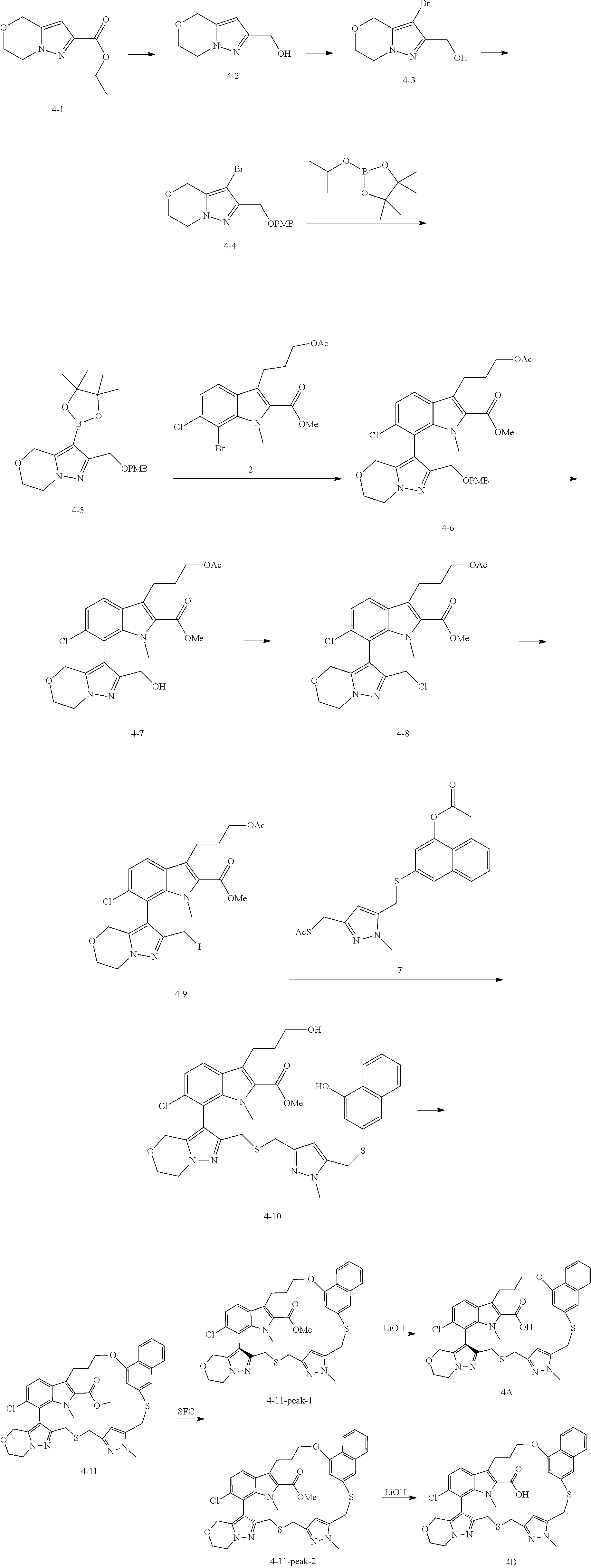

To a stirred solution of ethyl ethyl 6,7-dihydro-4H-pyrazolo[5,1-c][1,4]oxazine-2-carboxylate (4-1) (3.8 g, 19.3 mmol, 1 eq.) in THF (38 mL) was added LiAlH₄ (2.4 M in THF, 8.8 mL, 21.3 mmol, 1.1 eq.) at 0° C. and reaction mixture was stirred at RT for 1 h. After consumption of starting material, reaction mixture was quenched with ice cooled saturated Na₂SO₄ solution at 0° C. Resulting slurry was filtered through a Celite bed. The Celite bed was washed with ethyl acetate (60 mL). The filtrate was dried over Na₂SO₄, filtered and evaporated to get 2.9 g (18.8 mmol, 86%) of semi-pure (6,7-dihydro-4H-pyrazolo[5,1-c][1,4]oxazin-2-yl)methanol (4-2). The crude product was used without purification. MS (LCMS) m/z 155.12 [M+H]⁺. ¹H NMR (400 MHz, CDCl₃) δ 6.00 (s, 1H), 4.82 (s, 2H), 4.66 (s, 2H), 4.16 (t, J=5.2 Hz, 2H), 4.15-4.07 (m, 2H).

To a stirred solution of (6,7-dihydro-4H-pyrazolo[5,1-c][1,4]oxazin-2-yl)methanol (4-2, 2.9 g, 18.9 mmol, 1 eq.) in DCM (29 mL) was added NBS (3.69 g, 20.8 mmol, 1.1 eq.) at 0° C., and the mixture was stirred rt for 1 h. The reaction was quenched by the addition of water (30 mL), diluted with DCM (50 mL). The layers were separated and washed with brine (20 mL). The organic layer was dried over Na₂SO₄, filtered and evaporated to give the semi pure compound that was triturated with PE to afford to afford (3-bromo-6,7-dihydro-4H-pyrazolo[5,1-c][1,4]oxazin-2-yl) methanol (4-3, 3.8 g, 16.3 mmol, 86%) as a white solid. MS (LCMS) m/z=234.99 [M+H]⁺. ¹H NMR (400 MHz, CDCl₃) δ 4.73 (s, 2H), 4.67 (d, J=Hz, 2H), 4.16-4.13 (m, 2H), 4.11-4.08 (m, 2H), 2.03 (t, J=5.8 Hz, 1H).

To a stirred solution of (3-bromo-6,7-dihydro-4H-pyrazolo[5,1-c][1,4]oxazin-2-yl) methanol (4-3, 10 g, 43.1 mmol, 1 eq.) in DMF (100 mL) was added NaH (60% in oil, 2.06 g, 47.4 mmol, 1.1 eq.) at 0° C., and the mixture was stirred at rt for 30 min. 1-(Chloromethyl)-4-methoxybenzene (7.39 mL, 47.4 mmol, 1.1 eq.) followed by NaI (646 mg, 1.64 mmol, 0.1 eq.) were added, and the mixture was stirred at rt for 2 h. The reaction was quenched with ice and diluted with water (200 mL). The mixture was extracted with EtOAc (3×100 mL). The combined organic layer was washed with cold water (2×50 mL) and brine (2×50 mL). The organic layer was dried over Na₂SO₄, filtered and concentrated under reduced pressure. The crude was purified by silica gel column chromatography to give of semi pure 3-bromo-2-(((4-methoxybenzyl)oxy)methyl)-6,7-dihydro-4H-pyrazolo[5,1-c][1,4]oxazine (4-4, 12 g, 33.9 mmol, 79%). MS (LCMS) m/z 355.21 [M+H]⁺. ¹H NMR (400 MHz, CDCl₃) δ 7.33-7.30 (m, 2H), 6.89-6.86 (m, 2H), 4.73 (s, 2H), 4.54 (s, 2H), 4.49 (s, 2H), 4.16-4.14 (m, 2H), 4.10-4.07 (m, 2H), 3.80 (s, 3H).

To a stirred solution 3-bromo-2-(((4-methoxybenzyl)oxy)methyl)-6,7-dihydro-4H-pyrazolo[5,1-c][1,4]oxazine (4-4, 2 g, 5.66 mmol) in THF (20 mL) was added n-BuLi (1.6 M in hexanes, 4.6 mL, 7.36 mmol, 1.3 eq.) at −78° C., and the mixture was stirred at −78° C. for 50 min. 2-isopropoxy-4,4,5,5-tetramethyl-1,3,2-dioxaborolane (2.45 g, 11.33 mmol, 2 eq.) was added at −78° C. The temperature was slowly raised to rt and then stirred for 1 h. The solvents were evaporated. The residue was diluted with EtOAc (20 mL), filtered through a Celite pad. The filtrate was evaporated to afford 2-(((4-methoxybenzyl)oxy)methyl)-3-(4,4,5,5-tetramethyl-1,3,2-dioxaborolan-2-yl)-6,7-dihydro-4H-pyrazolo[5,1-c][1,4]oxazine (4-5, 2.1 g, crude) as a pale yellow solid. MS (LCMS) m/z 801.30 [2M+H]⁺.

To a stirred solution of 2-(((4-methoxybenzyl)oxy)methyl)-3-(4,4,5,5-tetramethyl-1,3,2-dioxaborolan-2-yl)-6,7-dihydro-4H-pyrazolo[5,1-c][1,4]oxazine (4-5, 2.1 g, 5.25 mmol, 1.0 eq.) in a mixture of 1,4-dioxane (22 mL) and water (4.4 mL) were added methyl methyl 3-(3-acetoxypropyl)-7-bromo-6-chloro-1-methyl-1H-indole-2-carboxylate (2, 1.76 g, 4.4 mmol, 0.8 eq.) and Cs₂CO₃ (3.57 g, 11 mmol, 2.1 eq.). The solution was degassed with Ar for 30 min. Dichloro[1 1′-bis(di-tert-butylphosphino)ferrocene]palladium(II) (286 mg, 0.44 mmol, 0.08 eq.) was added, and the solution was degassed again for 10 mins. The mixture was heated at 110° C. for 16 h. The mixture was cooled to rt, and the solvent was evaporated under reduced pressure. The brown residue diluted with EtOAc (150 mL), passed through a Celite pad and washed with EtOAc (50 mL). The filtrate was evaporated under reduced pressure, and the crude was purified by silica gel column chromatography using EtOAc and PE to afford methyl 3-(3-acetoxypropyl)-6-chloro-7-(2-(((4-methoxybenzyl)oxy)methyl)-6,7-dihydro-4H-pyrazolo[5,1-c][1,4]oxazin-3-yl)-1-methyl-1H-indole-2-carboxylate (4-6, 1.4 g, 2.34 mmol, 41% over 2-steps) as a brown liquid. MS (LCMS) m/z 596.34 [M+H]⁺.

To a stirred solution of methyl 3-(3-acetoxypropyl)-6-chloro-7-(2-(((4-methoxybenzyl)oxy)methyl)-6,7-dihydro-4H-pyrazolo[5,1-c][1,4]oxazin-3-yl)-1-methyl-1H-indole-2-carboxylate (4-6, 950 mg, 1.60 mmol, 1.0 eq.) in DCM (10 mL) was added TFA (1.3 mL, 16.0 mmol, 10 eq.) at 0° C., and the mixture was stirred at rt for 2 h. The solvent was evaporated. The residue was dissolved in EtOAc (30 mL) and washed with sat. NaHCO₃ (2×20 mL). The organic layer was collected, dried over Na₂SO₄, filtered and evaporated. The crude was purified by silica gel column chromatography using EtOAc in PE to afford methyl 3-(3-acetoxypropyl)-6-chloro-7-(2-(hydroxymethyl)-6,7-dihydro-4H-pyrazolo[5,1-c][1,4] oxazin-3-yl)-1-methyl-1H-indole-2-carboxylate (4-7, 430 mg, 0.9 mmol, 56%) as a brown colour gummy. MS (LCMS) m/z 476.22 [M+H]⁺. ¹H NMR (400 MHz, CDCl₃) δ 7.55 (d, J=8.8 Hz, 1H), 7.24 (d, J=8.8 Hz, 1H), 4.82 (s, 1H), 4.68 (s, 1H), 4.61-4.50 (m, 3H), 4.31-4.28 (m, 2H), 4.20-4.05 (m, 4H), 3.93 (s, 3H), 3.53 (s, 3H), 3.11-3.07 (m, 2H), 2.08 (s, 3H), 2.01-1.97 (m, 2H).

To a stirred solution of methyl 3-(3-acetoxypropyl)-6-chloro-7-(2-(hydroxymethyl)-6,7-dihydro-4H-pyrazolo[5,1-c][1,4] oxazin-3-yl)-1-methyl-1H-indole-2-carboxylate (4-7, 810 mg, 1.705 mmol, 1.0 eq.) in DCM (10 mL) under Ar was added SOCl₂ (0.13 mL, 1.875 mmol, 1.1 eq.) at 0° C., and the mixture was stirred at rt for 1 h. The solvent was evaporated to dryness to yield methyl 3-(3-acetoxypropyl)-6-chloro-7-(2-(chloromethyl)-5,6-dihydro-4H-pyrrolo[1,2-b]pyrazol-3-yl)-1-methyl-1H-indole-2-carboxylate (4-8, 830 mg), which used without further purification. MS (LCMS) m/z 494.16 [M+H]⁺.

To a stirred solution of methyl 3-(3-acetoxypropyl)-6-chloro-7-(2-(chloromethyl)-5,6-dihydro-4H-pyrrolo[1,2-b]pyrazol-3-yl)-1-methyl-1H-indole-2-carboxylate (4-8, 830 mg, 1.68 mmol, 1.0 eq.) in dry MeCN (50 mL) were added NaI (290 mg, 1.84 mmol, 1.1 eq.) at rt, and the mixture was heated to 70° C. for 2 h. The solvent was evaporated. The mixture was diluted with water (20 mL) and extracted with EtOAc (2×20 mL). The combined organic layer was dried over Na₂SO₄, filtered and evaporated to afford semi pure methyl-3-(3-acetoxypropyl)-6-chloro-7-(2-(iodomethyl)-5,6-dihydro-4H-pyrrolo[1,2-b]pyrazol-3-yl)-1-methyl-1H-indole-2-carboxylate (4-9, 830 mg) as a red colour gummy, which used without further purification. MS (LCMS) m/z 586.26 [M+H]⁺.

To a stirred solution of methyl-3-(3-acetoxypropyl)-6-chloro-7-(2-(iodomethyl)-5,6-dihydro-4H-pyrrolo[1,2-b]pyrazol-3-yl)-1-methyl-1H-indole-2-carboxylate (4-9) (830 mg, 1.41 mmol, 1.0 eq.) in MeOH (4 mL), THF (4 mL) were added K₂CO₃ (778 mg, 5.64 mmol, 4.0 eq.), and the solution was degassed with Ar for 20 min. In another round bottom flask, 3-(((3-((acetylthio)methyl)-1-methyl-1H-pyrazol-5-yl)methyl)thio)naphthalen-1-yl acetate (7, 625 mg, 1.56 mmol, 1.1 eq.) in methanol (4 mL) was degassed with Ar for 20 min, and this solution was added to previous mixture dropwise for 30 min. The mixture was stirred at rt for 16 h. The solvent was evaporated. The mixture was diluted with water (20 mL) and extracted with EtOAc (3×60 mL). The combined organic layer was dried over Na₂SO₄, filtered and evaporated to give semi pure 4-10 that was purified by silica gel column chromatography using 50-70% EtOAc in PE to afford methyl 6-chloro-7-(2-((((5-(((4-hydroxynaphthalen-2-yl)thio)methyl)-1-methyl-1H-pyrazol-3-yl)methyl)thio) methyl)-5,6-dihydro-4H-pyrrolo[1,2-b]pyrazol-3-yl)-3-(3-hydroxypropyl)-1-methyl-1H-indole-2-carboxylate (4-10, 430 mg, 0.59 mmol, 51% over 3-steps) as black color gummy. MS (LCMS) m/z 732.38 [M+H]⁺. ¹H NMR (400 MHz, CDCl₃) δ 8.26-8.24 (m, 1H), 7.77-7.32 (m, 1H), 7.65-7.60 (m, 2H), 7.52-7.45 (m, 3H), 7.26-7.23 (m, 1H), 6.57 9 (s, 1H), 5.97 (s, 1H), 4.62-4.52 (m, 2H), 4.33-4.30 (m, 2H), 3.94-3.92 (m, 6H), 3.71 (s, 1H), 3.70-3.65 (m, 6H), 3.55-2.45 (m, 4H), 3.16 (t, J=7.6 Hz, 2H), 2.00-1.90 (m, 2H), 1.25 (d, J=6.8 Hz, 3H).

To a stirred solution of TPP (673 mg, 2.57 mmol, 4.0 eq.) in toluene (4.7 mL) and THF (2 mL) was added a di-tert-butyl diazene-1,2-dicarboxylate (591 mg, 2.57 mmol, 4.0 eq.) at 0° C. After 10 min, methyl 6-chloro-7-(2-((((5-(((4-hydroxynaphthalen-2-yl)thio)methyl)-1-methyl-1H-pyrazol-3-yl)methyl)thio) methyl)-5,6-dihydro-4H-pyrrolo[1,2-b]pyrazol-3-yl)-3-(3-hydroxypropyl)-1-methyl-1H-indole-2-carboxylate (4-10, 470 mg, 0.642 mmol, 1.0 eq.) in THF (2.7 mL) was added, and the mixture was stirred at rt for 2 h. The mixture was diluted with water (20 mL) and extracted ethyl acetate (3×40 mL). The ethyl acetate layer was then washed with 1N aq. HCl (20 mL). The organic layer was dried over Na₂SO₄, filtered and evaporated to dryness. The crude was suspended in MeOH (10 mL) and sonicated for 10 min. The supernatant was decanted. The gummy solid was collected to yield semi-pure 4-11. The crude was purified by silica gel column to enrich the product. The crude (120 mg) was mixed with this batch and purified by prep. HPLC to yield Methyl (Z)-1⁶-chloro-1¹,6¹-dimethyl-2⁶,2⁷-dihydro-1¹H,2⁴H,6¹H-10-oxa-4,8-dithia-2(3,2)-pyrazolo[5,1-c][1,4]oxazina-1(7,3)-indola-6(3,5)-pyrazola-9(3,1)-naphthalenacyclo tridecaphane-1²-carboxylate (4-11, 260 mg. 0.36 mmol, 45%) as a white solid. MS (LCMS) m/z 714.38 [M+H]⁺. ¹H NMR (400 MHz, CDCl₃) δ 8.32-8.30 (m, 1H), 7.75-7.72 (m, 1H), 7.54-7.50 (m, 4H), 7.00-6.92 (m, 2H), 6.24 (s, 1H), 4.91 (s, 1H), 4.57 (s, 2H), 4.24-4.20 (m, 2H), 4.15-4.10 (m, 2H), 3.90-3.85 (m, 4H), 3.80-3.70 (m, 9H), 3.51-3.45 (m, 2H), 3.35-3.30 (m, 2H), 3.05 (d, J=14.0 Hz, 1H), 2.50-2.40 (m, 1H), 2.30-2.20 (m 1H).

Methyl (Z)-1⁶-chloro-1¹,6¹-dimethyl-2⁶,2⁷-dihydro-1¹H,2⁴H,6¹H-10-oxa-4,8-dithia-2(3,2)-pyrazolo[5,1-c][1,4]oxazina-1(7,3)-indola-6(3,5)-pyrazola-9(3,1)-naphthalen acyclotridecaphane-1²-carboxylate (4-11, 340 mg) was purified by SFC purification to afford 4-11-peak-1 (121 mg) as an off pale yellow solid with 89.5% of LCMS purity (chiral HPLC:99.95%) and 4-11-peak-2 (122 mg) as an off white solid with 86.5% of LCMS purity (chiral HPLC:98.68%). These two peaks were separately used for the next steps to get respective final compound.

4-11-peak-1: MS (LCMS) m/z 714.41 [M+H]⁺. Chiral SFC: 99.95%, RT=18.21 min (Column: Chiralpak IG (4.6*250 mm), 5 μm; Solvent A: “n-hexane; Solvent B: ethanol Isocratic (A:B): 50:50; Flow rate: 1 mL per min).

4-11-peak-2: MS (LCMS) m/z 714.45 [M+H]⁺. Chiral SFC: 99.95%, RT=18.21 min (Column: Chiralpak IG (4.6*250 mm), 5 μm; Solvent A: “n-hexane; Solvent B: ethanol Isocratic (A:B): 50:50; Flow rate: 1 mL per min).

To a stirred solution of methyl (Z)-1⁶-chloro-1¹,6¹-dimethyl-2⁶,2⁷-dihydro-1¹H,2⁴H,6¹H-10-oxa-4,8-dithia-2(3,2)-pyrazolo[5,1-c][1,4]oxazina-1(7,3)-indola-6(3,5)-pyrazola-9(3,1)-naphthalen acyclotridecaphane-1²-carboxylate (4-11-peak-1, 68 mg, 0.09 mmol) in THF:H₂O (1:1; 1.4 mL) was added LiOH.H₂O (80 mg, 1.90 mmol) at 0° C., and the mixture was stirred at rt for 16 h. The solvent was evaporated, and the aqueous layer was acidified to pH 2 using 2 N aq. HCl. The solid was filtered off and washed with water (5 mL). The semi-solid was collected, and purified by prep-TLC in 5% MeOH:DCM to afford (Z)-1⁶-chloro-1¹,6¹-dimethyl-2⁶,2⁷-dihydro-1¹H,2⁴H,6¹H-10-oxa-4,8-dithia-2(3,2)-pyrazolo[5,1-c][1,4]oxazina-1(7,3)-indola-6(3,5)-pyrazola-9(3,1)-naphthalenacyclotridecaphane-1²-carboxylic acid (4A, 21 mg, 31%, 0.051 mmol) as white solid. MS (LCMS) m/z 700.30 [M+H]⁺. ¹H NMR (400 MHz, DMSO-d₆) δ 8.07 (d, J=7.6 Hz, 1H), 7.78-7.69 (m, 2H), 7.50-7.37 (m, 2H), 7.35 (s, 1H), 7.06 (d, J=8.8 Hz, 1H), 6.73 (s, 1H), 4.92 (br s, 1H), 4.52-4.43 (m, 3H), 4.25-4.06 (m, 6H), 3.95-3.90 (m, 1H), 3.72 (s, 3H), 3.60-3.50 (m, 5H), 3.35-3.30 (m, 2H), 3.11 (d, J=14.0 Hz, 1H), 2.96-2.92 (m, 2H), 2.35-2.20 (m, 2H), LCMS purity: 96.31%; HPLC: 99.09%; Chiral purity: 99.98%.

To a stirred solution of methyl (Z)-1⁶-chloro-1¹,6¹-dimethyl-2⁶,2⁷-dihydro-1¹H,2⁴H,6¹H-10-oxa-4,8-dithia-2(3,2)-pyrazolo[5,1-c][1,4]oxazina-1(7,3)-indola-6(3,5)-pyrazola-9(3,1)-naphthalen acyclotridecaphane-1²-carboxylate (4-11-peak-2, 66 mg, 0.09 mmol) in THF:H₂O (1:1; 1.4 mL) was added LiOH.H₂O (80 mg, 1.90 mmol) at 0° C., and the mixture was stirred at rt for 16 h. The solvent was evaporated, and the aqueous layer was acidified to pH 2 using 2 N aq. HCl. The solid was filtered off and washed with water (5 mL). The semi-solid was collected, and purified by prep-TLC in 5% MeOH:DCM to afford (Z)-1⁶-chloro-1¹,6¹-dimethyl-2⁶,2⁷-dihydro-1¹H,2⁴H,6¹H-10-oxa-4,8-dithia-2(3,2)-pyrazolo[5,1-c][1,4]oxazina-1(7,3)-indola-6(3,5)-pyrazola-9(3,1)-naphthalenacyclotridecaphane-1²-carboxylic acid (4B, 44 mg, 68%, 0.063 mmol) as white solid. MS (LCMS) m/z 700.31 [M+H]⁺. ¹H NMR (400 MHz, DMSO-d₆) δ 8.07 (d, J=7.6 Hz, 1H), 7.72-7.68 (m, 2H), 7.50-7.40 (m, 2H), 7.34 (s, 1H), 7.05 (d, J=7.6 Hz, 1H), 6.75 (s, 1H), 4.96 (br s, 1H), 4.51-4.40 (m, 3H), 4.22-4.06 (m, 6H), 3.95-3.90 (m, 1H), 3.72 (s, 3H), 3.60-3.50 (m, 5H), 3.35-3.30 (m, 2H), 3.15-3.10 (m, 1H), 3.00-2.97 (m, 2H), 2.35-2.20 (m, 2H). LCMS purity: 99.06%; HPLC: 98.23%; Chiral purity: 99.09%.

The absolute stereochemistry of compounds (4A) and (4B) is arbitrarily assigned.

### Example 5

### (Z)-1⁶-Chloro-1¹,6¹-dimethyl-1¹H,2⁴H,2⁶H,6¹H-10-oxa-4,8-dithia-2(3,2)-pyrazolo[1,5-c]thiazola-1(7,3)-indola-6(3,5)-pyrazola-9(3,1)-naphthalenacyclotridecaphane-1²-carboxylic acid

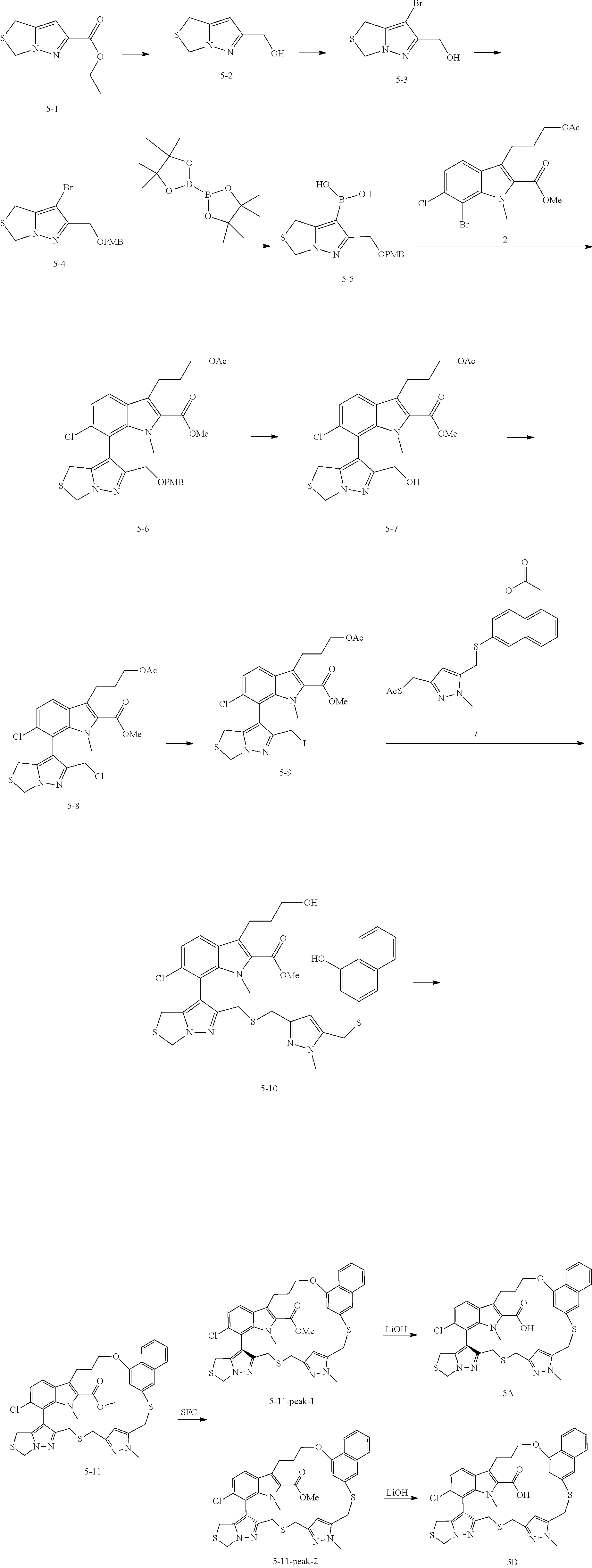

To a stirred solution of ethyl 4H,6H-pyrazolo[1,5-c]thiazole-2-carboxylate (5-1) (12.5 g, 63.1 mmol, 1 eq.) in THF (250 mL) was added LiAlH₄ (2.4 M in THF, 26 mL, 63.1 mmol, 1 eq.) at 0° C., and the mixture was stirred at rt for 1 h. After consumption of starting material, the reaction was quenched with an ice cooled sat. Na₂SO₄ solution at 0° C. The resulting slurry was filtered through Celite bed that was washed with 10% MeOH in DCM (3×100 mL). The filtrate was dried over Na₂SO₄, filtered and evaporated to dryness. The crude product was triturated with PE to give semi-pure (4H,6H-pyrazolo[1,5-c]thiazol-2-yl)methanol (5-2, 9 g, 57.6 mmol, 91%) as an off white solid, which was used without purification. MS (LCMS) m/z 157.01 [M+H]⁺. ¹H NMR (400 MHz, CDCl₃) δ 6.04 (s, 1H), 5.15 (t, J=1.8 Hz, 2H), 4.64 (s, 2H), 4.08 (s, 2H), 2.20 (br s, 1H).

To a stirred solution of (4H,6H-pyrazolo[1,5-c]thiazol-2-yl)methanol (5-2) (24.5 g, 157 mmol, 1 eq.) in DCM (250 mL) was added NBS (30.76 g, 172.8 mmol, 1.1 eq.) at 0° C. and stirred at rt for 1 h. The solvent was evaporated to dryness, and the residue was partitioned between water (500 mL) and ethyl acetate (1.5 L). The layers were separated, and the aqueous layer was further extracted with EtOAc (2×300 mL). The combined organic layer was washed with brine (1×200 mL). The organic layer was dried over Na₂SO₄, filtered and evaporated to give semi pure 5-3 which was triturated with PE to yield (3-bromo-4H,6H-pyrazolo[1,5-c]thiazol-2-yl)methanol (5-3, 27 g, 115.9 mmol, 73%) as a pale yellow solid. MS (LCMS) m/z 234.97 [M+H]⁺. ¹H NMR (400 MHz, CDCl₃) δ 5.20 (s, 2H), 4.64 (s, 2H), 4.03-4.02 (m, 2H), 1.85 (br s, 1H).

To a stirred solution of (3-bromo-4H,6H-pyrazolo[1,5-c]thiazol-2-yl)methanol (17.5 g, 74.46 mmol, 1 eq.) in DMF (175 mL) was added NaH (60% in oil, 3.28 g, 81.91 mmol, 1.1 eq.) at 0° C., and the mixture was stirred rt for 20 min. 1-(Chloromethyl)-4-methoxybenzene (11 mL, 81.91 mmol, 1.1 eq.) followed by KI (1.23 g, 7.45 mmol, 0.1 eq.) were then added. The mixture was stirred at rt for 1 h. The reaction was quenched with ice and diluted with water (200 mL). The precipitate was filtered off and washed with water (3×20 mL). The solid was collected and dried under reduced pressure to yield semi-pure 5-4. The crude product was purified by silica gel column chromatography to give 3-bromo-2-(((4-methoxybenzyl)oxy)methyl)-4H,6H-pyrazolo[1,5-c]thiazole (5-4, 12 g, 33.8 mmol, 45%) as a white solid. MS (LCMS) m/z 357.10 [M+H]⁺. ¹H NMR (400 MHz, CDCl₃) δ 7.33-7.29 (m, 2H), 6.89-6.86 (m, 2H), 5.21 (s, 2H), 4.53 (s, 2H), 4.46 (s, 2H), 4.20 (t, J=1.8 Hz, 2H), 3.80 (s, 3H).

A solution 3-bromo-2-(((4-methoxybenzyl)oxy)methyl)-4H,6H-pyrazolo[1,5-c]thiazole (3.5 g, 9.85 mmol) and bis(neopentylglycolato)diborane (10.05 g, 39.43 mmol, 4.0 eq.) in 1,4-dioxane (70 mL) was degassed for 20 min. To this mixture, KOAc (3.8 g, 39.43 mmol, 4.0 eq.) and Pd(dppf)Cl₂ DCM complex (803 mg, 0.985 mmol, 0.1 mmol) was added. The mixture was again degassed for 10 min, and then heated to 125° C. for 4 h. The mixture was filtered through a bed of Celite and washed with ethyl acetate (50 mL). The filtrate was concentrated to dryness. The crude was partitioned between 2N HCl (50 mL) and ethyl acetate (100 mL). The ethyl acetate layer was collected, dried over sodium sulphate, filtered and evaporated to dryness. The crude was purified by silica gel column chromatography to yield semi-pure product 5-5 (9.5 g) as pale yellow liquid. MS (LCMS) m/z 320.75 [M+H]⁺.

To a stirred solution of (2-(((4-methoxybenzyl)oxy)methyl)-4H,6H-pyrazolo[1,5-c]thiazol-3-yl)boronic acid (9 g, 28.13 mmol, 1.0 eq.) in a mixture of 1,4-dioxane (180 mL) and were added methyl 3-(3-acetoxypropyl)-7-bromo-6-chloro-1-methyl-1H-indole-2-carboxylate (2) (7.89 g, 19.68 mmol, 0.7 eq.) and Cs₂CO₃ (18.27 g, 56.25 mmol, 2.0 eq.). The solution was degassed with Ar for 20 min. To this mixture, dichloro[1 1′-bis(di-tert-butylphosphino)ferrocene]palladium(II). DCM complex (1.57 g, 2.79 mmol, 0.08 eq.) was added, and the mixture was degassed again for 10 min. The mixture was heated at 85° C. for 2 h. The mixture was cooled to rt. The mixture was filtered through a bed of Celite and washed with ethyl acetate (100 mL). The filtrate was collected, evaporated to dryness. The crude product was partitioned between EtOAc (200 mL) and water (100 mL). The layers were separated, and the aqueous layer was extracted with EtOAc (2×100 mL). The combined organic layer was dried over Na₂SO₄, filtered and evaporated to dryness. The crude was purified by silica gel column chromatography using EtOAc and PE to afford methyl 3-(3-acetoxypropyl)-6-chloro-7-(2-(((4-methoxybenzyl)oxy)methyl)-4H,6H-pyrazolo[1,5-c]thiazol-3-yl)-1-methyl-1H-indole-2-carboxylate (5-6, 4 g, 6.7 mmol) as a brown liquid. MS (LCMS) m/z 598.25 [M+H]⁺.

To a stirred solution of methyl 3-(3-acetoxypropyl)-6-chloro-7-(2-(((4-methoxybenzyl)oxy)methyl)-4H,6H-pyrazolo[1,5-c]thiazol-3-yl)-1-methyl-1H-indole-2-carboxylate (4 g, 6.7 mmol, 1.0 eq.) in DCM (10 mL) was added TFA (4 mL, 1 vol) at 0° C., and the mixture was stirred at rt for 2 h. The solvent was evaporated, and the residue was dissolved in EtOAc (100 mL) and washed with saturated NaHCO₃ (2×20 mL). The organic layer was collected, dried over Na₂SO₄, filtered and evaporated. The crude product was purified by silica gel column chromatography using EtOAc in PE to afford methyl 3-(3-acetoxypropyl)-6-chloro-7-(2-(hydroxymethyl)-4H,6H-pyrazolo[1,5-c] thiazol-3-yl)-1-methyl-1H-indole-2-carboxylate (5-7, 1 g, 2.1 mmol, 32% over 3-steps) as a brown color gummy. MS (LCMS) m/z 478.33 [M+H]⁺. ¹H NMR (400 MHz, CDCl₃) δ 7.58 (d, J=8.4 Hz, 2H), 7.24 (d, J=8.4 Hz, 2H), 5.15 (s, 1H), 4.65 (s, 1H), 4.55-4.45 (m, 2H), 4.15-4.10 (m, 3H), 4.08-3.95 (s, 1H), 3.92 (s, 3H), 3.87-3.80 (m, 1H), 3.58 (s, 3H), 3.10-3.05 (m, 2H), 2.08 (s, 3H).

To a stirred solution of Methyl 3-(3-acetoxypropyl)-6-chloro-7-(2-(hydroxymethyl)-4H,6H-pyrazolo[1,5-c] thiazol-3-yl)-1-methyl-1H-indole-2-carboxylate (700 mg, 1.46 mmol, 1.0 eq.) in DCM (7 mL) under Ar was added SOCl₂ (261 mg, 2.20 mmol, 1.5 eq.) at 0° C., and the mixture was stirred at rt for 2 h. The solvent was evaporated to dryness. The residue was partitioned between EtOAc (30 mL) and sat. NaHCO₃ (20 mL). The layers were separated, and the aqueous layer was further extracted with EtOAc (2×30 mL). The combined organic layer was dried over Na₂SO₄, filtered and evaporated to yield crude 5-8 (700 mg), which was used without purification. MS (LCMS) m/z 496.13 [M+H]⁺.

To a stirred solution of Methyl 3-(3-acetoxypropyl)-6-chloro-7-(2-(chloromethyl)-4H,6H-pyrazolo[1,5-c]thiazol-3-yl)-1-methyl-1H-indole-2-carboxylate (5-8) (700 mg, 1.41 mmol, 1.0 eq.) in dry MeCN (7 mL) were added NaI (412 mg, 2.82 mmol, 2.0 eq.) at rt, and the mixture was heated to 85° C. for 3 h. The solvent was evaporated, and the mixture was diluted with ice cold water (10 mL) and extracted with EtOAc (3×30 mL). The combined organic layer was dried over Na₂SO₄, filtered and evaporated to afford semi pure methyl 3-(3-acetoxypropyl)-6-chloro-7-(2-(iodomethyl)-4H,6H-pyrazolo[1,5-c]thiazol-3-yl)-1-methyl-1H-indole-2-carboxylate (5-9, 720 mg) as a dark yellow liquid that was used without further purification. MS (LCMS) m/z 588.20 [M+H]⁺.

To a stirred solution of methyl 3-(3-acetoxypropyl)-6-chloro-7-(2-(iodomethyl)-4H,6H-pyrazolo[1,5-c]thiazol-3-yl)-1-methyl-1H-indole-2-carboxylate (5-9) (720 mg, 1.22 mmol, 1.0 eq.) in MeOH (3.2 mL), THF (7.2 mL) were added K₂CO₃ (673 mg, 4.88 mmol, 4.0 eq.) and degassed with Ar for 20 min. In another round bottom flask, 3-(((3-((acetylthio)methyl)-1-methyl-1H-pyrazol-5-yl)methyl)thio)naphthalen-1-yl acetate (7) (489 mg, 1.22 mmol, 1.1 eq.) in methanol (4 mL) was degassed with Ar for 20 min, and this solution was added to previous mixture dropwise for 30 min. The mixture stirred at rt for 3 h. The solvent was evaporated, and the mixture was diluted with water (20 mL) and extracted with EtOAc (3×60 mL). The combined organic layer was dried over Na₂SO₄, filtered and evaporated to get semi pure 5-10 that was purified by silica gel column chromatography using 50-70% EtOAc in PE to afford methyl 6-chloro-7-(2-((((5-(((4-hydroxynaphthalen-2-yl)thio)methyl)-1-methyl-1H-pyrazol-3-yl)methyl)thio)methyl)-4H,6H-pyrazolo[1,5-c]thiazol-3-yl)-3-(3-hydroxypropyl)-1-methyl-1H-indole-2-carboxylate (5-10, 350 mg, 0.48 mmol, 34% over 3-steps) as pale yellow solid. MS (LCMS) m/z 734.49 [M+H]⁺. ¹H NMR (400 MHz, CDCl₃) δ 9.17 (br s, 1H), 8.26-8.20 (m, 1H), 7.77-7.70 (m, 1H), 7.65-7.60 (m, 2H), 7.52-7.45 (m, 3H), 7.26-7.20 (m, 2H), 6.54 (s, 1H), 5.97 (s, 1H), 5.35-5.30 (m, 1H), 4.15-4.10 (m, 1H), 4.00-3.90 (m, 6H), 3.80-3.45 (m, 12H), 3.20-3.10 (m, 2H), 2.00-1.90 (m, 2H),

To a stirred solution of TPP (482 mg, 1.84 mmol, 3.0 eq.) in toluene (4.5 mL) and THF (2 mL) was added a di-tert-butyl diazene-1,2-dicarboxylate (424 mg, 1.84 mmol, 3.0 eq.) at 0° C. After 10 min, methyl 6-chloro-7-(2-((((5-(((4-hydroxynaphthalen-2-yl)thio)methyl)-1-methyl-1H-pyrazol-3-yl)methyl)thio)methyl)-4H,6H-pyrazolo[1,5-c]thiazol-3-yl)-3-(3-hydroxypropyl)-1-methyl-1H-indole-2-carboxylate (450 mg, 0.614 mmol, 1.0 eq.) in THF (2.5 mL) was added. The mixture was stirred at rt for 16 h. The solvent was evaporated, and the residue suspended in ice cold water (10 mL). The solid was filtered off and washed with water (5 mL). The solid was further purified by silica gel column to yield semi-pure methyl (Z)-1⁶-chloro-1¹,6¹-dimethyl-1¹H,2⁴H,2⁶H,6¹H-10-oxa-4,8-dithia-2(3,2)-pyrazolo[1,5-c]thiazola-1(7,3)-indola-6(3,5)-pyrazola-9(3,1)-naphthalenacyclotridecaphane-1²-carboxylate (5-11, 400 mg) as a white solid that was then purified by prep HPLC to yield methyl (Z)-1⁶-chloro-1¹,6¹-dimethyl-1¹H,2⁴H,2⁶H,6¹H-10-oxa-4,8-dithia-2(3,2)-pyrazolo[1,5-c]thiazola-1(7,3)-indola-6(3,5)-pyrazola-9(3,1)-naphthalenacyclotridecaphane-1²-carboxylate (5-11, 260 mg, 0.36 mmol, 45%) as a white solid. MS (LCMS) m/z 716.28 [M+H]⁺. HPLC 99.8%.

Methyl (Z)-1⁶-chloro-1¹,6¹-dimethyl-1¹H,2⁴H,2⁶H,6¹H-10-oxa-4,8-dithia-2(3,2)-pyrazolo[1,5-c]thiazola-1(7,3)-indola-6(3,5)-pyrazola-9(3,1)-naphthalenacyclotride caphane-1²-carboxylate (340 mg) was purified by SFC purification to afford 5-11-peak-1 (150 mg) as an off pale yellow solid with 99.26% of LCMS purity (chiral HPLC:99.66%) and 5-11-peak-2 (115 mg) as an off white solid with 97.94% of LCMS purity (chiral HPLC:99.89%). These two peaks were separately used for the next steps to get respective final compound.

5-11-peak-1: MS (LCMS) m/z 716.28 [M+H]⁺. Chiral SFC: 99.26%, RT=13.922 min (Column: Chiralpak IG (4.6*250 mm), 5 μm; Solvent A: “n-hexane; Solvent B: ethanol Isocratic (A:B): 50:50; Flow rate: 1 mL per min).

5-11-peak-2: MS (LCMS) m/z 716.36 [M+H]⁺. Chiral SFC: 99.89%, RT=19.805 min (Column: Chiralpak IG (4.6*250 mm), 5 μm; Solvent A: “n-hexane; Solvent B: ethanol Isocratic (A:B): 50:50; Flow rate: 1 mL per min).

To a stirred solution of methyl (Z)-1⁶-chloro-1¹,6¹-dimethyl-1¹H,2⁴H,2⁶H,6¹H-10-oxa-4,8-dithia-2(3,2)-pyrazolo[1,5-c]thiazola-1(7,3)-indola-6(3,5)-pyrazola-9(3,1)-naphthalenacyclotrid ecaphane-1²-carboxylate (5-11-peak-1) (120 mg, 0.17 mmol, 1.0 eq.) in THF:H₂O (1:1; 1.4 mL) was added LiOH.H₂O (78 mg, 3.4 mmol, 20 eq.) at 0° C. and stirred at 80° C. for 1 h. The solvent was evaporated, and the aqueous layer was acidified to pH 2 using 2 N aqueous HCl. The solid was filtered off and washed with water (5 mL). The solid was collected dried under vacuum to afford (Z)-1⁶-chloro-1¹,6¹-dimethyl-1¹H,2⁴H,2⁶H,6¹H-10-oxa-4,8-dithia-2(3,2)-pyrazolo[1,5-c]thiazola-1(7,3)-indola-6(3,5)-pyrazola-9(3,1)-naphthalenacyclotridecaphane-1²-carboxylic acid (5A, 100 mg, 84%, 0.14 mmol) as a white solid. MS (LCMS) m/z 702.22 [M+H]⁺. ¹H NMR (400 MHz, DMSO-d₆) δ 8.10 (d, J=7.6 Hz, 1H), 7.87 (d, J=8.8 Hz, 1H), 7.73 (d, J=7.2 Hz, 1H), 7.51-7.41 (m, 2H), 7.39 (s, 1H), 7.12 (d, J=8.4 Hz, 1H), 6.49 (s, 1H), 5.35-5.25 (m, 2H), 4.80 (s, 1H), 4.26 (s, 2H), 4.10-4.02 (m, 1H), 4.00-3.90 (m, 1H), 3.90-3.80 (2H), 3.70 (s, 3H), 3.57 (s, 3H), 3.50-3.40 (m, 2H), 3.25-3.20 (m, 2H), 3.15-3.05 (m, 2H), 2.90 (d, J=14 Hz, 1H), 2.40-2.30 (m 1H), 2.30-2.15 (m, 1H). LCMS purity: 99.27%; HPLC: 98.77%; Chiral purity: 99.35%.

To a stirred solution of methyl (Z)-1⁶-chloro-1¹,6¹-dimethyl-1¹H,2⁴H,2⁶H,6¹H-10-oxa-4,8-dithia-2(3,2)-pyrazolo[1,5-c]thiazola-1(7,3)-indola-6(3,5)-pyrazola-9(3,1)-naphthalenacyclotrid ecaphane-1²-carboxylate (5-11-Peak-2) (95 mg, 0.13 mmol) in THF:H₂O (1:1; 1.4 mL) was added LiOH.H₂O (63 mg, 2.65 mmol) at 0° C. and stirred at rt for 16 h. The solvent was evaporated, and the aqueous layer was acidified to pH 2 using 2 N aqueous HCl. The solid was filtered off and washed with water (5 mL). The solid was collected to afford ((Z)-1⁶-chloro-1¹,6¹-dimethyl-1¹H,2⁴H,2⁶H,6¹H-10-oxa-4,8-dithia-2(3,2)-pyrazolo[1,5-c]thiazola-1(7,3)-indola-6(3,5)-pyrazola-9(3,1)-naphthalenacyclotridecaphane-1²-carboxylic acid (5B, 80 mg, 86%, 0.11 mmol) as a white solid. MS (LCMS) m/z 702.15 [M+H]⁺. ¹H NMR (400 MHz, DMSO-d₆) δ 8.09 (d, J=7.6 Hz, 1H), 7.80 (d, J=8.4 Hz, 1H), 7.71 (d, J=7.6 Hz, 1H), 7.51-7.41 (m, 2H), 7.37 (s, 1H), 7.09 (d, J=8.8 Hz, 1H), 6.69 (s, 1H), 5.31-5.25 (m, 2H), 4.88 (s, 1H), 4.36-4.26 (s, 1H), 4.25-4.20 (m, 1H), 4.10-4.02 (m, 1H), 4.00-3.80 (m, 3H), 3.71 (s, 3H), 3.55 (s, 3H), 3.50-3.40 (m, 2H), 3.25-3.20 (m, 2H), 3.10-2.90 (m, 3H), 2.40-2.20 (m 2H); LCMS purity: 98.44%. HPLC: 98.82%; Chiral purity: 99.96%.

The absolute stereochemistry of compounds (5A) and (5B) is arbitrarily assigned.

### Example 6

### (Z)-1⁶-Chloro-1¹,2⁵,6¹-trimethyl-1¹H,2¹H,6¹H-10-oxa-4,8-dithia-1(7,3)-indola-2(4,3),6(3,5)-dipyrazola-9(3,1)-naphthalenacyclotridecaphane-1²-carboxylic acid

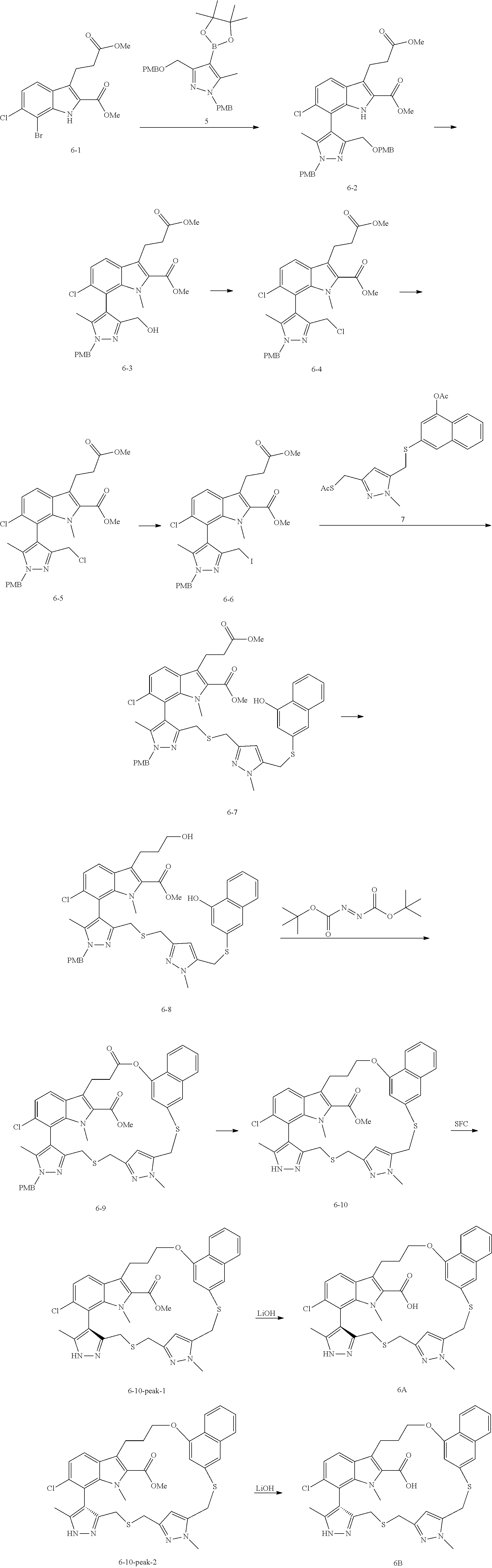

To the degassed solution of 1-(4-methoxybenzyl)-3-(((4-methoxybenzyl)oxy)methyl)-5-methyl-4-(4,4,5,5-tetramethyl-1,3,2-dioxaborolan-2-yl)-1H-pyrazole (5) (7 g, 14.64 mmol) in 1,4-dioxane:water (56 mL, 14 mL) were added methyl 7-bromo-6-chloro-3-(3-methoxy-3-oxopropyl)-1H-indole-2-carboxylate (6-1) (5.460 g, 14.64 mmol) and Cs₂CO₃ (9.516 g, 29.28 mmol). The solution was degassed with Ar for 20 mins. Dichloro[1 1′-bis(di-tert-butylphosphino)ferrocene] palladium(II) (571 mg, 0.878 mmol) was added, and the solution was degassed again for 10 min. The mixture was heated at 100° C. for 2 h. The mixture was diluted with water (200 mL) and extracted with EtOAc (3×200 mL). The combined organic layers were dried over anhydrous Na₂SO₄, filtered and concentrated under reduced pressure. The product was purified by silica gel column chromatography and eluted using 30% EtOAc in PE to afford methyl 6-chloro-3-(3-methoxy-3-oxopropyl)-7-(1-(4-methoxybenzyl)-3-(((4-methoxybenzyl) oxy)methyl)-5-methyl-1H-pyrazol-4-yl)-1H-indole-2-carboxylate (6-2, 7.5 g, 11.62 mmol, 79%) as a brown viscous liquid. MS (ESI) m/z 646.34 [M+1]⁺. ¹H NMR (400 MHz, CDCl₃) δ 7.63 (d, J=8.8 Hz, 1H), 7.26-7.23 (m, 2H), 7.11 (d, J=8.8 Hz, 2H), 7.01-6.98 (m, 2H), 6.90-6.86 (m 2H), 6.76-6.73 (m, 2H), 5.41-5.25 (m, 2H), 4.44-4.36 (m, 3H), 4.18 (d, J=11.2 Hz, 1H), 3.80 (s, 3H), 3.78 (s, 3H), 3.73 (s, 3H), 3.66 (s, 3H), 3.44-3.38 (m, 2H), 2.72 (t, J=7.8 Hz, 2H), 2.02 (s, 3H).

To a stirred solution of methyl 6-chloro-3-(3-methoxy-3-oxopropyl)-7-(1-(4-methoxybenzyl)-3-(((4-methoxybenzyl)oxy)methyl)-5-methyl-1H-pyrazol-4-yl)-1H-indole-2-carboxylate (7.5 g, 11.62 mmol) in dry DMF (75 mL) were added Cs₂CO₃ (4.534 g, 13.953 mmol) and Mel (1.448 mL, 23.255 mmol). The mixture was stirred at rt for 2.5 h. The reaction was quenched with water (250 mL) and extracted with EtOAc (3×500 mL). The organic layer was separated, dried over anhydrous Na₂SO₄, filtered and evaporated to afford methyl 6-chloro-3-(3-methoxy-3-oxopropyl)-7-(1-(4-methoxybenzyl)-3-(((4-methoxybenzyl)oxy)methyl)-5-methyl-1H-pyrazol-4-yl)-1-methyl-1H-indole-2-carboxylate (6-3, 7.5 g, 11.3 mmol, 97%) as a yellow liquid that was used without further purification. MS (ESI) m/z 660.61 [M+1]⁺. ¹H NMR (400 MHz, CDCl₃) δ 7.62 (d, J=8.8 Hz, 1H), 7.26-7.22 (m, 1H), 7.08 (d, J=8.4 Hz, 2H), 6.88-6.80 (m, 4H), 6.69-6.64 (m, 2H), 5.40-5.28 (m, 2H), 4.40-4.30 (m, 3H), 4.21 (d, J=11.6 Hz, 1H), 3.90 (s, 3H), 3.80 (s, 3H), 3.75 (s, 3H), 3.67 (s, 3H), 3.55 (br s, 1H), 3.48 (s, 3H), 3.40-3.30 (m, 2H), 2.66 (t, J=7.8 Hz, 2H), 1.95 (s, 2H).

To a stirred solution of methyl 6-chloro-3-(3-methoxy-3-oxopropyl)-7-(1-(4-methoxybenzyl)-3-(((4-methoxybenzyl)oxy)methyl)-5-methyl-1H-pyrazol-4-yl)-1-methyl-1H-indole-2-carboxylate (8.5 g, 12.898 mmol) in DCM (130 mL) was added TFA (7 mL, 90.28 mmol) at 0° C., and the mixture was stirred at rt for 2 h. The mixture was diluted with DCM (200 mL), washed with a sat. aq. NaHCO₃ solution (2×100 mL) and brine (2×100 mL). The organic layer was separated, dried over anhydrous Na₂SO₄, filtered and evaporated to give a semi pure compound (8.4 g). This semi pure compound was dissolved in MeOH (78 mL) and K₂CO₃ (3.39 g, 24.566 mmol) was added at rt, and the mixture was stirred for 2 h. The mixture was diluted with CH₂Cl₂ (200 mL), washed with water (2×100 mL), dried over anhydrous Na₂SO₄, filtered and concentrated under reduced pressure to afford the crude compound that was purified by silica gel column chromatography and eluted at 30% EtOAc in PE to afford methyl 6-chloro-7-(3-(hydroxymethyl)-1-(4-methoxybenzyl)-5-methyl-1H-pyrazol-4-yl)-3-(3-methoxy-3-oxopropyl)-1-methyl-1H-indole-2-carboxylate (6-4, 4.0 g, 7.42 mmol, 57%) as a yellow liquid. MS (ESI) m/z 540.26 [M+1]⁺. ¹H NMR (400 MHz, CDCl₃) δ 7.63 (d, J=8.8 Hz, 1H), 7.26-7.24 (m, 1H), 7.08 (d, J=8.8 Hz, 2H), 6.88-6.85 (m, 2H), 5.38 (m, 2H), 4.52-4.48 (m, 2H), 3.92 (s, 3H), 3.80 (s, 3H), 3.67 (s, 3H), 3.51 (s, 3H), 3.35-3.30 (m, 2H), 2.65 (t, J=7.8 Hz, 2H), 1.95 (s, 3H), 1.86 (br s, 1H).

To a stirred solution of methyl 6-chloro-7-(3-(hydroxymethyl)-1-(4-methoxybenzyl)-5-methyl-1H-pyrazol-4-yl)-3-(3-methoxy-3-oxopropyl)-1-methyl-1H-indole-2-carboxylate (4 g, 7.42 mmol) in DCM (40 mL) under Ar was added SOCl₂ (0.645 mL, 8.90 mmol) at 0° C., and the mixture was stirred at rt for 30 min. The mixture was diluted with DCM (100 mL) and washed with a sat. NaHCO₃ solution (2×50 mL). The organic layer was separated, dried over anhydrous Na₂SO₄, filtered and evaporated to afford semi pure methyl 6-chloro-7-(3-(chloromethyl)-1-(4-methoxybenzyl)-5-methyl-1H-pyrazol-4-yl)-3-(3-methoxy-3-oxopropyl)-1-methyl-1H-indole-2-carboxylate (6-5, 4.1 g, 7.36 mmol, LCMS; 97%) as a light yellow liquid that was used without further purification. MS (ESI) m/z 558.26 [M+1]⁺.

To a stirred solution of semi pure methyl 6-chloro-7-(3-(chloromethyl)-1-(4-methoxybenzyl)-5-methyl-1H-pyrazol-4-yl)-3-(3-methoxy-3-oxopropyl)-1-methyl-1H-indole-2-carboxylate (4.1 g, 7.36 mmol) in dry MeCN (90 mL) were added NaI (1.985 g, 13.24 mmol) at rt, and the mixture was heated to 80° C. for 2.5 h. The solvent was evaporated, and the mixture was diluted with water (200 mL) and extracted with EtOAc (2×200 mL). The organic layer was separated, dried over Na₂SO₄, filtered and evaporated to afford semi pure methyl 6-chloro-7-(3-(iodomethyl)-1-(4-methoxybenzyl)-5-methyl-1H-pyrazol-4-yl)-3-(3-methoxy-3-oxopropyl)-1-methyl-1H-indole-2-carboxylate (6-6, 4,3 g, 6.625 mmol, 90%; LCMS: 93%) as a light yellow liquid that was used without further purification. MS (ESI) m/z 650.34 [M+1]⁺. ¹H NMR (400 MHz, CDCl₃) δ 7.66 (d, J=8.4 Hz, 1H), 7.28-7.26 (m, 1H), 7.07 (d, J=8.8 Hz, 2H), 6.89-6.85 (m, 2H), 5.36-5.24 (m, 2H), 4.29 (d, J=10.0 Hz, 1H), 4.22 (d, J=10.4 Hz, 1H), 3.92 (s, 3H), 3.80 (s, 3H), 3.67 (s, 3H), 3.49 (s, 3H), 3.37-3.32 (m, 2H), 2.68-2.64 (m, 2H), 1.93 (s, 3H).

To a stirred solution of semi pure methyl 6-chloro-7-(3-(iodomethyl)-1-(4-methoxybenzyl)-5-methyl-1H-pyrazol-4-yl)-3-(3-methoxy-3-oxopropyl)-1-methyl-1H-indole-2-carboxylate (4 g, 6.163 mmol) in degassed MeOH (40 mL) were added K₂CO₃ (2.03 g, 14.718 mmol) and degassed with Ar for 10 min. In another round bottom flask, 3-(((3-((acetylthio)methyl)-1-methyl-1H-pyrazol-5-yl)methyl)thio)naphthalen-1-yl acetate (7) (2.465 g, 6.163 mmol) in methanol (40 mL) was degassed with Ar for 10 min, and this solution was added to previous mixture dropwise. The mixture was stirred at rt for 16 h. The mixture was diluted with CH₂Cl₂ (300 mL) and washed with water (100 mL) and brine (100 mL). The separated organic layer was dried over Na₂SO₄, filtered and evaporated to give a semi pure compound that was purified by silica gel column chromatography using 50% EtOAc in PE to afford methyl 6-chloro-7-(3-((((5-(((4-hydroxynaphthalen-2-yl)thio)methyl)-1-methyl-1H-pyrazol-3-yl)methyl)thio)methyl)-1-(4-methoxybenzyl)-5-methyl-1H-pyrazol-4-yl)-3-(3-methoxy-3-oxopropyl)-1-methyl-1H-indole-2-carboxylate (6-7, 5 g, 5.97 mmol, 80% over three steps) as an off-white solid. MS (ESI) m/z 838.50 [M+1]⁺. ¹H NMR (400 MHz, DMSO-d₆) δ 10.20 (br s, 1H), 8.03 (d, J=10.0 Hz, 1H), 7.71 (t, J=10.8 Hz, 2H), 7.46-7.34 (m, 2H), 7.32-7.24 (m, 2H), 7.12-7.06 (m, 2H), 6.94-6.86 (m, 2H), 6.78 (br s, 1H), 5.90 (s, 1H), 5.29 (s, 2H), 4.27 (s, 2H), 3.84 (s, 3H), 3.80-3.70 (m, 7H), 3.68 (s, 3H), 3.53 (s, 3H), 3.40-3.35 (m, 3H, merged with solvent residual peak) 3.28-3.22 (m, 2H), 2.60-2.50 (m, 2H), 1.90 (s, 3H).

To a stirred solution of methyl 6-chloro-7-(3-((((5-(((4-hydroxynaphthalen-2-yl)thio)methyl)-1-methyl-1H-pyrazol-3-yl)methyl)thio)methyl)-1-(4-methoxybenzyl)-5-methyl-1H-pyrazol-4-yl)-3-(3-methoxy-3-oxopropyl)-1-methyl-1H-indole-2-carboxylate (5.4 g, 6.451 mmol) in THF (40 mL) was added BH₃.THF (1M in THF) (32.2 mL, 32.25 mmol) at 0° C., and the mixture was heated at 75° C. for 16 h. The mixture was cooled to 0° C. The reaction was quenched with MeOH (5 mL) and 4N HCl (10 mL). The mixture was stirred for 30 min and then extracted with CH₂Cl₂ (2×500 mL). The organic layer was washed with aq. NaHCO₃ solution, dried over anhydrous Na₂SO₄, filtered and concentrated to give a semi pure compound that was purified by silica gel column chromatography using 65% EtOAc in PE to afford methyl 6-chloro-7-(3-((((5-(((4-hydroxynaphthalen-2-yl)thio)methyl)-1-methyl-1H-pyrazol-3-yl)methyl)thio)methyl)-1-(4-methoxybenzyl)-5-methyl-1H-pyrazol-4-yl)-3-(3-hydroxypropyl)-1-methyl-1H-indole-2-carboxylate (6-8, 2.4 g, 2.966 mmol, 46%) as an off-white solid. MS (ESI) m/z 810.49 [M+1]⁺. ¹H NMR (400 MHz, CDCl₃) δ 9.70 (br s, 1H), 8.18-8.15 (m, 1H), 7.72 (d, J=7.6 Hz, 1H), 7.65-7.60 (m, 2H), 7.50-7.40 (m, 2H), 7.26-7.24 (m, 2H), 7.09 (d, J=8.8 Hz, 2H), 6.83-6.79 (m, 2H), 6.54 (s, 1H), 5.91 (s, 1H), 5.40-5.27 (m, 2H), 3.95-3.90 (m, 5H), 3.77 (s, 3H), 3.67-3.65 (m, 2H), 3.62 (s, 1H), 3.55-3.40 (m, 9H), 3.20-3.13 (m, 2H), 2.00-1.93 (m, 5H).

To a stirred solution of methyl 6-chloro-7-(3-((((5-(((4-hydroxynaphthalen-2-yl)thio)methyl)-1-methyl-1H-pyrazol-3-yl)methyl)thio)methyl)-1-(4-methoxybenzyl)-5-methyl-1H-pyrazol-4-yl)-3-(3-hydroxypropyl)-1-methyl-1H-indole-2-carboxylate (3×800 mg, 0.988 mmol) in toluene (10 mL) and THF (1 mL) were added TPP (1.294 g, 4.94 mmol) and di-tert-butyl diazene-1,2-dicarboxylate (1.130 g, 4.94 mmol). The mixture was stirred at rt for 2 h. The reaction was quenched with water (50 mL) and extracted with EtOAc (2×50 mL). The organic layer was dried over Na₂SO₄, filtered, concentrated to give semi pure 6-9 that was triturated with MeOH (10 mL) to afford methyl (Z)-1⁶-chloro-2¹-(4-methoxybenzyl)-1¹,2⁵,6¹-trimethyl-1¹H,2¹H,6¹H-10-oxa-4,8-dithia-1(7,3)-indola-2(4,3),6(3,5)-dipyrazola-9(3,1)-naphthalenacyclotridecaphane-1²-carboxylate (6-9, 600 mg, 0.758 mmol, 25%) as an off-white solid. MS (ESI) m/z 792.46 [M+1]⁺.

In a pressure tube, to a stirred solution of afford methyl (Z)-1⁶-chloro-2¹-(4-methoxybenzyl)-1¹,2⁵,6¹-trimethyl-1¹H,2¹H,6¹H-10-oxa-4,8-dithia-1(7,3)-indola-2(4,3),6(3,5)-dipyrazola-9(3,1)-naphthalenacyclotridecaphane-1²-carboxylate (600 mg, 0.758 mmol) in TFA (10 mL) was added anisole (0.49 g, 4.548 mmol) at rt, and the mixture was stirred at 100° C. for 40 h. The mixture was concentrated under reduced pressure, and the obtained crude compound was triturated with MeOH (3 mL) to afford methyl (Z)-1⁶-chloro-1¹,2⁵,6¹-trimethyl-1¹H,2¹H,6¹H-10-oxa-4,8-dithia-1(7,3)-indola-2(4,3),6(3,5)-dipyrazola-9(3,1)-naphthalenacyclotridecaphane-1²-carboxylate (6-10, 360 mg, 0.536 mmol, 70%) as an off-white solid. MS (ESI) m/z 672.30 [M+1]⁺. ¹H NMR (400 MHz, DMSO-d₆) δ 12.63 (br s, 1H), 8.14-8.08 (m, 1H), 7.94-7.84 (m, 1H), 7.76-7.65 (m, 1H), 7.53-7.44 (m, 2H), 7.40 (s, 1H), 7.20-7.10 (m, 1H), 6.64 (br s, 1H), 4.78 (s, 1H) 4.30-4.20 (m, 2H), 4.15-4.05 (m, 2H), 3.84 (s, 3H), 3.83-3.73 (m, 1H), 3.68 (s, 3H), 3.50-3.35 (m, 4H), 3.20-3.00 (m, 4H), 2.40-2.30 (m, 1H), 2.28-2.20 (m, 1H), 1.98 (br s, 3H).

Methyl (Z)-1⁶-chloro-1¹,2⁵,6¹-trimethyl-1¹H,2¹H,6¹H-10-oxa-4,8-dithia-1(7,3)-indola-2(4,3),6(3,5)-dipyrazola-9(3,1)-naphthalenacyclotridecaphane-1²-carboxylate (6-10, 360 mg) was purified by SFC to afford 6-10-peak-1 (120 mg) as an off white solid with 94% of LCMS purity (chiral HPLC: 99.87%) and 6-10-peak-2 (117 mg) as an off white solid with 98% of LCMS purity (chiral HPLC: 99.31%). These two peaks were separately used for the next steps to get respective final compound.

6-10-peak-1: MS (ESI) m/z 672.30 [M+1]⁺. ¹H NMR (400 MHz, DMSO-d₆) δ 12.63 (br s, 1H), 8.14-8.08 (m, 1H), 7.92-7.85 (m, 1H), 7.76-7.65 (m, 1H), 7.52-7.45 (m, 2H), 7.40 (s, 1H), 7.20-7.10 (m, 1H), 6.64 (br s, 1H), 4.78 (s, 1H), 4.30-4.20 (m, 2H), 4.15-4.05 (m, 2H), 3.85-3.73 (m, 4H), 3.68 (s, 3H), 3.50-3.35 (m, 5H), 3.20-3.00 (m, 3H), 2.40-2.30 (m, 1H), 2.28-2.20 (m, 1H), 1.95 (br s, 3H). LCMS purity: 93.97%; Chiral HPLC: 99.87%.

6-10-peak-2: MS (ESI) m/z 672.30 [M+1]⁺. ¹H NMR (400 MHz, DMSO-d₆) δ 12.63 (br s, 1H), 8.14-8.08 (m, 1H), 7.90-7.85 (m, 1H), 7.76-7.65 (m, 1H), 7.52-7.45 (m, 2H), 7.40 (s, 1H), 7.20-7.10 (m, 1H), 6.62 (br s, 1H), 4.78 (s, 1H), 4.30-4.20 (m, 2H), 4.15-4.05 (m, 2H), 3.85-3.73 (m, 4H), 3.68 (s, 3H), 3.50-3.35 (m, 5H), 3.20-3.00 (m, 3H), 2.43-2.30 (m, 1H), 2.28-2.20 (m, 1H), 1.95 (br s, 3H). LCMS purity: 98.16%; Chiral HPLC: 99.31%.

To a stirred solution of methyl (Z)-1⁶-chloro-1¹,2⁵,6¹-trimethyl-1¹H,2¹H,6¹H-10-oxa-4,8-dithia-1(7,3)-indola-2(4,3),6(3,5)-dipyrazola-9(3,1)-naphthalenacyclotridecaphane-1²-carboxylate (6-10-peak-1) (120 mg, 0.178 mmol) in THF:H₂O (1:1, 8 mL) was added LiOH.H₂O (149 mg, 3.56 mmol) at 0° C., and the mixture was stirred at rt for 20 h. The mixture was concentrated under reduced pressure, and the residue was acidified to pH 2 using 2 N aqueous HCl (5 mL). The mixture was extracted with EtOAc (2×25 mL). The combined organic layers were dried over anhydrous Na₂SO₄, filtered and concentrated under reduced pressure to afford crude 5A that was purified by Prep-TLC eluted using 7% MeOH in DCM to afford 5A. Compound 5A was dissolved in ACN (1 mL), water (1 mL), frozen and then lyophilized to afford (Z)-1⁶-chloro-1¹,2⁵,6¹-trimethyl-1¹H,2¹H,6¹H-10-oxa-4,8-dithia-1(7,3)-indola-2(4,3),6(3,5)-dipyrazola-9(3,1)-naphthalenacyclotridecaphane-1²-carboxylic acid (5A, 50 mg, 0.076 mmol, 43%) as a white solid. MS (ESI) m/z 658.26 [M+1]⁺. ¹H NMR (400 MHz, DMSO-d₆) δ 12.63 (br s, 1H), 8.08 (d, J=8.0 Hz, 1H), 7.77-7.70 (m, 2H), 7.50-7.40 (m, 2H), 7.36 (s, 1H), 7.07 (d, J=8.4 Hz, 1H), 6.70 (s, 1H), 4.87 (s, 1H), 4.40-4.30 (m, 1H), 4.25-4.20 (m, 1H), 4.10-4.05 (m, 1H), 3.95-3.85 (m, 1H), 3.71 (s, 3H), 3.52-3.45 (m, 6H), 3.20-3.10 (m, 4H), 3.05-2.95 (m, 1H), 2.92 (d, J=14.0 Hz, 1H), 2.35-2.20 (m, 2H), 1.94 (s, 3H); LCMS purity: 99.48%. HPLC purity: 99.49% and Chiral HPLC: 99.22%.

To a stirred solution of methyl (Z)-1⁶-chloro-1¹,2⁵,6¹-trimethyl-1¹H,2¹H,6¹H-10-oxa-4,8-dithia-1(7,3)-indola-2(4,3),6(3,5)-dipyrazola-9(3,1)-naphthalenacyclotridecaphane-1²-carboxylate (6-10-peak-2) (117 mg, 0.174 mmol) in THF:H₂O (1:1, 8 mL) was added LiOH.H₂O (146 mg, 3.48 mmol) at 0° C., and the mixture was stirred at rt for 20 h. The mixture was concentrated under reduced pressure, and the residue was acidified to pH 2 using 2 N aqueous HCl (5 mL). The mixture was extracted with EtOAc (2×25 mL). The combined organic layers were dried over anhydrous Na₂SO₄, filtered and concentrated under reduced pressure to afford crude 5B that was purified by Prep-TLC eluted at 7% MeOH in DCM to afford 5B. Compound 5B dissolved in ACN (1 mL), water (1 mL), frozen and then lyophilized to afford (Z)-1⁶-chloro-1¹,2⁵,6¹-trimethyl-1¹H,2¹H,6¹H-10-oxa-4,8-dithia-1(7,3)-indola-2(4,3),6(3,5)-dipyrazola-9(3,1)-naphthalenacyclotridecaphane-1²-carboxylic acid (5B, 42 mg, 0.063 mmol, 36%) as a white solid. MS (ESI) m/z 658.26 [M+1]⁺. ¹H NMR (400 MHz, DMSO-d₆) δ 12.63 (br s, 1H), 8.08 (d, J=8.0 Hz, 1H), 7.77-7.70 (m, 2H), 7.52-7.43 (m, 2H), 7.36 (s, 1H), 7.07 (d, J=8.0 Hz, 1H), 6.70 (s, 1H), 4.89 (s, 1H), 4.40-4.30 (m, 1H), 4.25-4.20 (m, 1H), 4.10-4.05 (m, 1H), 3.95-3.85 (m, 1H), 3.71 (s, 3H), 3.52-3.45 (m, 6H), 3.20-3.10 (m, 4H), 3.05-2.95 (m, 1H), 2.92 (d, J=14.0 Hz, 1H), 2.35-2.20 (m, 2H), 1.94 (s, 3H); LCMS purity: 98.31%. HPLC purity: 98.50% and Chiral HPLC: 99.04%.

The absolute stereochemistry of compounds (6A) and (6B) is arbitrarily assigned.

### Example 7

### (Z)-1⁶-Fluoro-6¹-isopropyl-1¹,2⁵-dimethyl-1¹H,2¹H,6¹H-10-oxa-4,8-dithia-1(7,3)-indola-2(4,3),6(3,5)-dipyrazola-9(3,1)-naphthalenacyclotridecaphane-1²-carboxylic acid

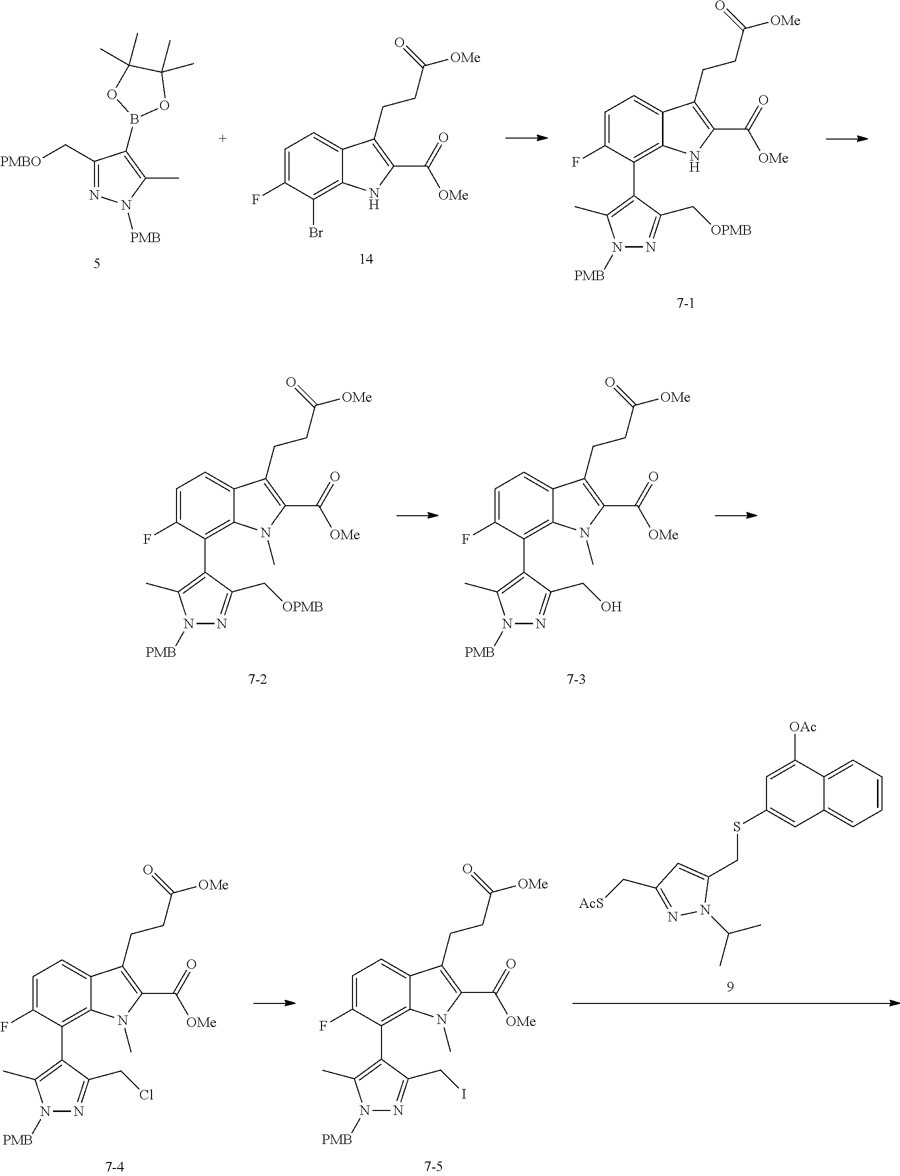

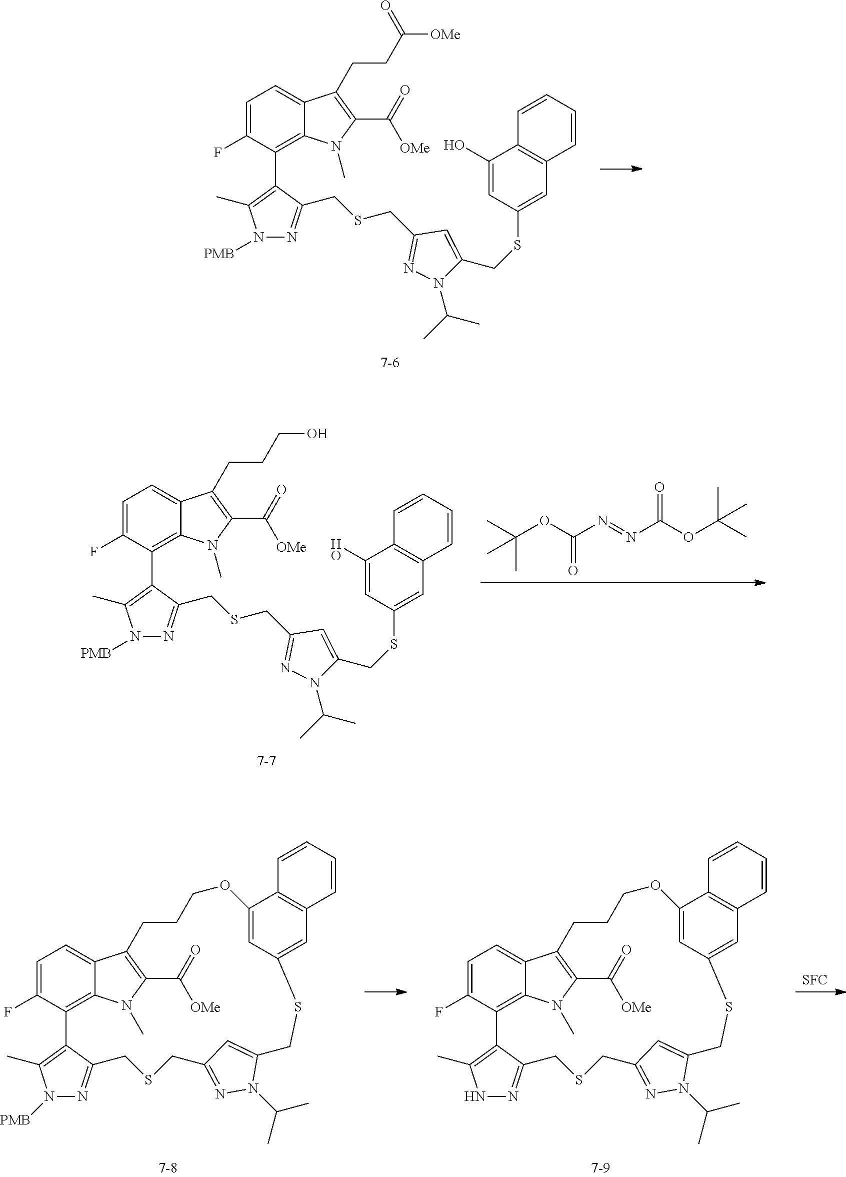

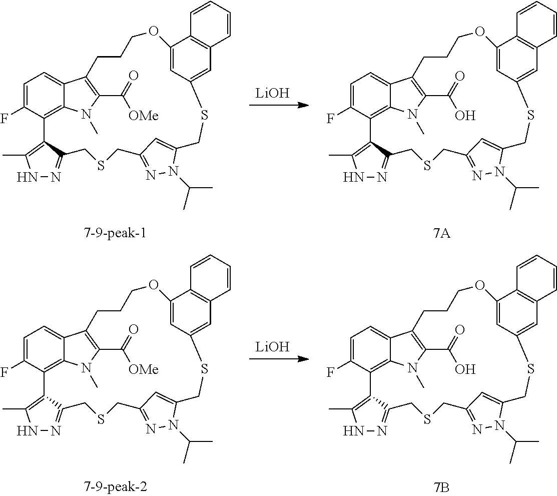

To the degassed solution of 1-(4-methoxybenzyl)-3-(((4-methoxybenzyl)oxy)methyl)-5-methyl-4-(4,4,5,5-tetramethyl-1,3,2-dioxaborolan-2-yl)-1H-pyrazole (5) (7 g, 14.64 mmol) in 1,4-dioxane:water (56 mL, 14 mL) were added methyl 7-bromo-6-chloro-3-(3-methoxy-3-oxopropyl)-1H-indole-2-carboxylate (14) (5.460 g, 14.64 mmol) and Cs₂CO₃ (9.516 g, 29.28 mmol). The solution was degassed with Ar for 20 mins. Dichloro[1 1′-bis(di-tert-butylphosphino)ferrocene] palladium(II) (571 mg, 0.878 mmol) was added, and the mixture was degassed again for 10 min. The mixture was heated at 100° C. for 2 h. The mixture was diluted with water (200 mL) and extracted with EtOAc (3×200 mL). The combined organic layers were dried over anhydrous Na₂SO₄, filtered and concentrated under reduced to obtain a crude that was purified by silica gel column chromatography eluting using 30% EtOAc in PE to afford methyl 6-chloro-3-(3-methoxy-3-oxopropyl)-7-(1-(4-methoxybenzyl)-3-(((4-methoxybenzyl)oxy)methyl)-5-methyl-1H-pyrazol-4-yl)-1H-indole-2-carboxylate (7-1, 7.5 g, 11.62 mmol, 79%) as a brown viscous liquid. MS (ESI) m/z 646.34 [M+1]. ¹H NMR (400 MHz, CDCl₃) δ 9.2 (s, 1H), 7.63 (d, J=8.8 Hz, 1H), 7.25-7.23 (m, 1H), 7.11 (d, J=8.8 Hz, 2H), 6.98 (d, J=3.2 Hz, 2H), 6.86 (d, J=5.2 Hz, 2H), 6.76 (d, J=4.8 Hz, 2H), 5.38 (d, J=15.6 Hz, 1H), 5.31-5.27 (m, 1H), 4.43-4.35 (m, 3H), 4.18 (d, J=11.2 Hz, 1H), 3.80 (s, 3H), 3.78 (s, 3H), 3.73 (s, 3H), 3.66 (s, 3H), 3.44-3.38 (m, 2H), 2.71 (t, J=7.8 Hz, 2H), 2.02 (s, 3H).

To a stirred solution of methyl 6-chloro-3-(3-methoxy-3-oxopropyl)-7-(1-(4-methoxybenzyl)-3-(((4-methoxybenzyl)oxy)methyl)-5-methyl-1H-pyrazol-4-yl)-1H-indole-2-carboxylate (4.5 g, 7.142 mmol) in dry DMF (40 mL) were added Cs₂CO₃ (3.48 g, 10.71 mmol) and Mel (0.88 mL, 14.28 mmol), and the mixture was stirred at rt for 2.5 h. The reaction was quenched with water (150 mL) and extracted with EtOAc (3×500 mL). The organic layer was separated, dried over anhydrous Na₂SO₄, filtered and evaporated to afford methyl 6-fluoro-3-(3-methoxy-3-oxopropyl)-7-(1-(4-methoxybenzyl)-3-(((4-methoxybenzyl)oxy)methyl)-5-methyl-1H-pyrazol-4-yl)-1H-indole-2-carboxylate (7-2, 4.5 g, 6.99 mmol,) as a yellow liquid that was used without further purification. MS (ESI) m/z 644.46 [M+1]⁺.

To a stirred solution of methyl 6-fluoro-3-(3-methoxy-3-oxopropyl)-7-(1-(4-methoxybenzyl)-3-(((4-methoxybenzyl)oxy)methyl)-5-methyl-1H-pyrazol-4-yl)-1-methyl-1H-indole-2-carboxylate (3.6 g, 5.592 mmol) in DCM (40 mL) was added TFA (4.1 mL, 54.372 mmol) at 0° C., and the mixture was stirred at rt for 2 h. The mixture was diluted with DCM (200 mL) and washed with a sat. aq. NaHCO₃ solution (2×100 mL) and brine (2×100 mL). The organic layer was separated, dried over anhydrous Na₂SO₄, filtered and evaporated to give semi pure (4.5 g). The semi pure (4.5 g) was dissolved in MeOH (25 mL) and TEA (5 mL) was added at rt, and the mixture was stirred for 2 h. The mixture was diluted with CH₂Cl₂ (200 mL), washed with water (2×100 mL), dried over anhydrous Na₂SO₄, filtered and concentrated under reduced pressure to afford the crude that was purified by silica gel column chromatography eluting at 30% EtOAc in PE to afford methyl 6-fluoro-7-(3-(hydroxymethyl)-1-(4-methoxybenzyl)-5-methyl-1H-pyrazol-4-yl)-3-(3-methoxy-3-oxopropyl)-1-methyl-1H-indole-2-carboxylate (7-3, 2.5 g, mmol, 57%) as a yellow liquid. MS (ESI) m/z 540.26 [M+1]. ¹H NMR (400 MHz, CDCl₃) δ 7.64 (dd, J=8.8 Hz, 5.2 Hz, 1H), 7.09 (d, J=8.8 Hz, 2H), 6.98 (t, J=8.0 Hz, 1H), 6.85 (d, J=8.8 Hz, 2H), 5.34 (d, J=15.6 Hz, 1H), 5.26 (d, J=15.6 Hz, 1H), 4.53 (d, J=5.2 Hz, 2H), 3.91 (s, 3H), 3.80 (s, 3H), 3.67 (s, 3H), 3.53 (s, 3H), 3.33 (t, J=8 Hz, 2H), 2.65 (t, J=7.8 Hz, 2H), 2.08 (t, J=6 Hz, 1H), 1.95 (s, 3H).

To a stirred solution of methyl 6-fluoro-7-(3-(hydroxymethyl)-1-(4-methoxybenzyl)-5-methyl-1H-pyrazol-4-yl)-3-(3-methoxy-3-oxopropyl)-1-methyl-1H-indole-2-carboxylate (2.5 g, 4.7750 mmol) in DCM (40 mL) under Ar was added SOCl₂ (0.4 mL, 5.730 mmol) at 0° C., and the mixture was stirred at rt for 30 min. The mixture was diluted with DCM (300 mL) and washed with a sat. NaHCO₃ solution (100 mL). The organic layer was separated, dried over anhydrous Na₂SO₄, filtered and evaporated to afford semi pure methyl 7-(3-(chloromethyl)-1-(4-methoxybenzyl)-5-methyl-1H-pyrazol-4-yl)-6-fluoro-3-(3-methoxy-3-oxopropyl)-1-methyl-1H-indole-2-carboxylate (7-4, 2.6 g, 4.797 mmol) as a light yellow liquid that was used without further purification. MS (ESI) m/z 542.1 [M+1]⁺.

To a stirred solution of semi pure methyl 7-(3-(chloromethyl)-1-(4-methoxybenzyl)-5-methyl-1H-pyrazol-4-yl)-6-fluoro-3-(3-methoxy-3-oxopropyl)-1-methyl-1H-indole-2-carboxylate (2.6 g, 4.797 mmol) in dry MeCN (50 mL) were added NaI (1.4 g, 9.594 mmol) rt, and the mixture was heated to 80° C. for 2.5 h. The solvent was evaporated, The mixture was diluted with water (200 mL) and extracted with EtOAc (2×250 mL). The organic layer was separated, dried over Na₂SO₄, filtered and evaporated to afford semi pure methyl 6-fluoro-7-(3-(iodomethyl)-1-(4-methoxybenzyl)-5-methyl-1H-pyrazol-4-yl)-3-(3-methoxy-3-oxopropyl)-1-methyl-1H-indole-2-carboxylate (7-5, 3.0 g, 4.735 mmol,) as a light yellow liquid that was used without further purification. MS (ESI) m/z 634.28 [M+1]⁺.

To a stirred solution of semi pure methyl 6-fluoro-7-(3-(iodomethyl)-1-(4-methoxybenzyl)-5-methyl-1H-pyrazol-4-yl)-3-(3-methoxy-3-oxopropyl)-1-methyl-1H-indole-2-carboxylate (3 g, 4.739 mmol) in degassed MeOH (30 mL) were added K₂CO₃ (719 mg, 5.212 mmol) and degassed with Ar for 10 min. In another round bottom flask, 3-(((3-((acetylthio)methyl)-1-isopropyl-1H-pyrazol-5-yl)methyl)thio)naphthalen-1-yl acetate (9) (2.02 g, 4.739 mmol) in methanol (30 mL) was degassed with Ar for 10 min, and this solution was added to previous mixture dropwise. The mixture was stirred at rt for 16 h. The mixture was diluted with ethyl acetate (300 mL) and washed with water (100 mL) and brine (100 mL). The organic layer was dried over Na₂SO₄, filtered and evaporated to give a semi pure compound that was purified by silica gel column chromatography using 100% EtOAc in PE to afford methyl 6-fluoro-7-(3-((((5-(((4-hydroxynaphthalen-2-yl)thio)methyl)-1-isopropyl-1H-pyrazol-3-yl)methyl)thio)methyl)-1-(4-methoxybenzyl)-5-methyl-1H-pyrazol-4-yl)-3-(3-methoxy-3-oxopropyl)-1-methyl-1H-indole-2-carboxylate (7-6, 2.2 g, 2.588 mmol, 40% over three steps) as an off-white solid. MS (ESI) m/z 850.56 [M+1]⁺.

To a stirred solution of methyl 6-fluoro-7-(3-((((5-(((4-hydroxynaphthalen-2-yl)thio)methyl)-1-isopropyl-1H-pyrazol-3-yl)methyl)thio)methyl)-1-(4-methoxybenzyl)-5-methyl-1H-pyrazol-4-yl)-3-(3-methoxy-3-oxopropyl)-1-methyl-1H-indole-2-carboxylate (2 g, 2.355 mmol) in THF (20 mL) was added BH₃.THF (1M in THF) (12.9 mL, 12.9 mmol) at 0° C., and the mixture was heated at 75° C. for 16 h. The mixture was cooled to 0° C., and the reaction was quenched with MeOH (5 mL) and 4N HCl (10 mL). The mixture was stirred for 30 min and extracted with ethyl acetate (2×500 mL). The organic layer was washed with an aq. NaHCO₃ solution, dried over anhydrous Na₂SO₄, filtered and concentrated to give a semi pure compound that was purified by silica gel column chromatography using 70% EtOAc in PE to afford methyl 6-fluoro-7-(3-((((5-(((4-hydroxynaphthalen-2-yl)thio)methyl)-1-isopropyl-1H-pyrazol-3-yl)methyl)thio)methyl)-1-(4-methoxybenzyl)-5-methyl-1H-pyrazol-4-yl)-3-(3-hydroxypropyl)-1-methyl-1H-indole-2-carboxylate (7-7, 1.2 g, 1.459 mmol, 52%) as an off-white solid. MS (ESI) m/z 822.59 [M+1]⁺.

To a stirred solution of TPP (701 mg, 2.679 mmol) in toluene (10 mL) was added a solution of di-tert-butyl diazene-1,2-dicarboxylate (616 mg, 2.679 mmol), methyl 6-fluoro-7-(3-((((5-(((4-hydroxynaphthalen-2-yl)thio)methyl)-1-isopropyl-1H-pyrazol-3-yl)methyl)thio)methyl)-1-(4-methoxybenzyl)-5-methyl-1H-pyrazol-4-yl)-3-(3-hydroxypropyl)-1-methyl-1H-indole-2-carboxylate (1.1 g, 1.339 mmol) in THF (10 mL). The mixture was stirred at 90° C. for 2 h. The reaction was quenched with water (50 mL), extracted with EtOAc (2×50 mL). The organic layer was dried over Na₂SO₄, filtered, concentrated which was purified by silica gel column (100-200) purification using 100% EtOAc to afford semi pure 7-8 that was purified by normal prep-HPLC to afford methyl (Z)-1⁶-fluoro-6¹-isopropyl-2¹-(4-methoxybenzyl)-1¹,2⁵-dimethyl-1¹H,2¹H,6¹H-10-oxa-4,8-dithia-1(7,3)-indola-2(4,3),6(3,5)-dipyrazola-9(3,1)-naphthalenacyclotridecaphane-1²-carboxylate (7-8, 580 mg, 0.721 mmol, 54%) as an off white solid. MS (LCMS) m/z 804.51 [M+H.

In a pressure tube, to a stirred solution of afford methyl (Z)-1⁶-fluoro-6¹-isopropyl-2¹-(4-methoxybenzyl)-1¹,2⁵-dimethyl-1¹H,2¹H,6¹H-10-oxa-4,8-dithia-1(7,3)-indola-2(4,3),6(3,5)-dipyrazola-9(3,1)-naphthalenacyclotridecaphane-1²-carboxylate (580 mg, 0.7213 mmol) in TFA (10 mL) was added anisole (468 mg, 4.328 mmol) at rt, and the mixture was stirred at 120° C. for 40 h. The mixture was concentrated under reduced pressure and a crude was obtained. The mixture was diluted with ethyl acetate (200 mL), washed with aq. NaHCO₃ solution, dried over anhydrous Na₂SO₄, filtered and concentrated to give a semi pure compound that was purified by silica gel column chromatography using 40% EtOAc in PE to afford methyl (Z)-1⁶-fluoro-6¹-isopropyl-1¹,2⁵-dimethyl-1¹H,2¹H,6¹H-10-oxa-4,8-dithia-1(7,3)-indola-2(4,3),6(3,5)-dipyrazola-9(3,1)-naphthalenacyclotridecaphane-1²-carboxylate (7-9, 280 mg, 0.409 mmol, 56%) as an off-white solid. MS (ESI) m/z 684.45 [M+1]⁺.

Methyl (Z)-1⁶-fluoro-6¹-isopropyl-1¹,2⁵-dimethyl-1¹H,2¹H,6¹H-10-oxa-4,8-dithia-1(7,3)-indola-2(4,3),6(3,5)-dipyrazola-9(3,1)-naphthalenacyclotridecaphane-1²-carboxylate (7-9, 280 mg) was purified by SFC purification to afford 7-9-peak-1 (90 mg) as an off white solid with 97.7% of LCMS purity (chiral HPLC: 99.98%) and 7-9-peak-2 (95 mg) as an off white solid with 94% of LCMS purity (chiral HPLC: 99.78%). These two peaks were separately used for the next steps to get respective final compound.

7-9-peak-1: MS (LCMS) m/z 684.56 [M+H]⁺. ¹H NMR (400 MHz, CDCl₃) δ 9.98 (br s, 1H), 8.15 (brs, 1H), 7.67-7.58 (m, 2H), 7.47-7.45 (m, 2H), 7.29 (brs, 1H), 6.71 (t, J=8.8 Hz, 1H), 6.36 (s, 1H), 4.88 (s, 1H), 4.50-4.45 (m, 1H), 3.91-3.81 (m, 7H), 3.61-3.49 (m, 5H), 3.22-3.14 (m, 4H), 2.45-2.33 (m, 2H), 2.17 (s, 3H), 1.49 (d, J=6.4 Hz, 3H), 1.41 (d, J=6.8 Hz, 3H).

7-9-peak-2: MS (LCMS) m/z 684.56 [M+H]. ¹H NMR (400 MHz, CDCl₃) δ 9.98 (br s, 1H), 8.15 (brs, 1H), 7.67-7.58 (m, 2H), 7.47-7.45 (m, 2H), 7.29 (brs, 1H), 6.71 (t, J=8.8 Hz, 1H), 6.36 (s, 1H), 4.88 (s, 1H), 4.50-4.45 (m, 1H), 3.91-3.81 (m, 7H), 3.61-3.49 (m, 5H), 3.22-3.14 (m, 4H), 2.45-2.33 (m, 2H), 2.17 (s, 3H), 1.49 (d, J=6.4 Hz, 3H), 1.41 (d, J=6.8 Hz, 3H).

To a nitrogen degassed solution of methyl (Z)-1⁶-fluoro-6¹-isopropyl-1¹,2⁵-dimethyl-1¹H,2¹H,6¹H-10-oxa-4,8-dithia-1(7,3)-indola-2(4,3),6(3,5)-dipyrazola-9(3,1)-naphthalenacyclotridecaphane-1²-carboxylate (7-9-peak-1, 90 mg, 0.131 mmol) in MeOH:THF:H₂O (1:1:1, 1 mL) was added LiOH.H₂O (83 mg, 01.976 mmol) at rt, and the mixture was stirred at 70° C. for 3 h. The solvent was evaporated, and the mixture was diluted with water (1 mL), acidified to pH 2 using 1N aqueous HCl. The mixture was filtered, and (Z)-1⁶-fluoro-6¹-isopropyl-1¹,2⁵-dimethyl-1¹H,2¹H,6¹H-10-oxa-4,8-dithia-1(7,3)-indola-2(4,3),6(3,5)-dipyrazola-9(3,1)-naphthalenacyclotridecaphane-1²-carboxylic acid (7-9-peak-1, 81 mg, 0.122 mmol, 81%) was obtained as an off white solid. MS (LCMS) m/z 670.20 [M+H]⁺. ¹H NMR (400 MHz, DMSO-d₆) δ 13.2 (brs, 1H), 8.08 (d, J=7.2 Hz, 1H), 7.92 (dd, J=8.8, 5.6 Hz, 1H), 7.71 (d, J=7.6 Hz, 1H), 7.49-7.43 (m, 2H), 7.33 (s, 1H), 6.89 (t, J=8.8 Hz, 1H), 6.72 (s, 1H), 4.74 (s, 1H), 4.62-4.59 (m, 1H), 4.28 (brs, 2H), 4.18-4.14 (m, 1H), 3.91-3.87 (m, 1H), 3.52 (s, 3H), 3.22-2.99 (m, 6H), 2.49-2.23 (m, 2H), 1.97 (s, 3H), 1.32 (q, J=6.4 Hz, 6H).

To a nitrogen degassed solution of methyl (Z)-1⁶-fluoro-6¹-isopropyl-1¹,2⁵-dimethyl-1¹H,2¹H,6¹H-10-oxa-4,8-dithia-1(7,3)-indola-2(4,3),6(3,5)-dipyrazola-9(3,1)-naphthalenacyclotridecaphane-1²-carboxylate (7-9-peak-2, 95 mg, 0.138 mmol) in MeOH:THF:H₂O (1:1:1, 1 mL) was added LiOH.H₂O (86 mg, 2.07 mmol) at rt, and the mixture was stirred at 70° C. for 3 h. The solvent was evaporated, and the mixture was diluted with water (1 mL), acidified to pH 2 using 1N aqueous HCl. The mixture was filtered, and (Z)-1⁶-fluoro-6¹-isopropyl-1¹,2⁵-dimethyl-1¹H,2¹H,6¹H-10-oxa-4,8-dithia-1(7,3)-indola-2(4,3),6(3,5)-dipyrazola-9(3,1)-naphthalenacyclotridecaphane-1²-carboxylic acid (7-9-peak-2, 81 mg, 0.122 mmol, 92%) was obtained as an off white solid. MS (LCMS) m/z 670.15 [M+H]⁺. ¹H NMR (400 MHz, DMSO-d₆) δ 13.2 (brs, 1H), 8.08 (d, J=7.2 Hz, 1H), 7.92 (dd, J=8.8, 5.6 Hz, 1H), 7.71 (d, J=7.6 Hz, 1H), 7.49-7.43 (m, 2H), 7.33 (s, 1H), 6.89 (t, J=8.8 Hz, 1H), 6.72 (s, 1H), 4.74 (s, 1H), 4.62-4.59 (m, 1H), 4.28 (brs, 2H), 4.18-4.14 (m, 1H), 3.91-3.87 (m, 1H), 3.52 (s, 3H), 3.22-2.99 (m, 6H), 2.49-2.23 (m, 2H), 1.97 (s, 3H), 1.31 (q, J=6.4 Hz, 6H).

The absolute stereochemistry of compounds (7A) and (7B) is arbitrarily assigned.

### Example 8

### (Z)-1⁶-Chloro-6¹-isopropyl-2¹-(4-methoxybenzyl)-1¹,2⁵-dimethyl-1¹H,2¹H,6¹H-10-oxa-4,8-dithia-1(7,3)-indola-2(4,3),6(3,5)-dipyrazola-9(3,1)-naphthalenacyclotridecaphane-1²-carboxylic acid

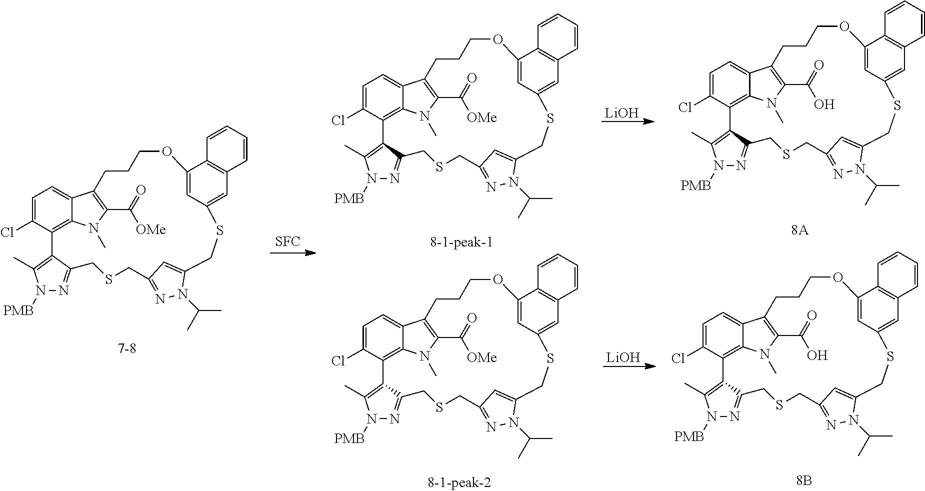

From example 7, racemic methyl (Z)-1⁶-chloro-6¹-isopropyl-2¹-(4-methoxybenzyl)-1¹,2⁵-dimethyl-1¹H,2¹H,6¹H-10-oxa-4,8-dithia-1(7,3)-indola-2(4,3),6(3,5)-dipyrazola-9(3,1)-naphthalenacyclotridecaphane-1²-carboxylate (7-8, 55 mg) was purified by SFC purification to afford 8-1-peak-1 (21 mg) as an off white solid with 92% of LCMS purity (chiral HPLC: 99.9%) and 8-1-peak-2 (21 mg) as an off white solid with 99% of LCMS purity (chiral HPLC: 99.7%). These two peaks were separately used for the next steps to get respective final compound.

8-1-peak-1: MS (ESI) m/z 820.46 [M+1]⁺. LCMS purity: 92.87%; Chiral purity: 99.97%.

8-1-Peak-2: MS (ESI) m/z 820.46 [M+1]⁺. LCMS purity: 99.84%; Chiral purity: 99.72%.

To a stirred solution of methyl (Z)-1⁶-chloro-6¹-isopropyl-2¹-(4-methoxybenzyl)-1¹,2⁵-dimethyl-1¹H,2¹H,6¹H-10-oxa-4,8-dithia-1(7,3)-indola-2(4,3),6(3,5)-dipyrazola-9(3,1)-naphthalenacyclotridecaphane-1²-carboxylate (8-1-peak-1, 21 mg, 0.0256 mmol) in degassed MeOH:THF:H₂O (1:1:1, 3 mL) was added LiOH.H₂O (16 mg, 0.384 mmol) at rt, and the mixture was heated at 60° C. for 1 h. The mixture was concentrated under reduced pressure, and the residue was acidified to pH-3 using 0.5 N aqueous HCl. The solid formed was filtered, and washed with water and pentane to afford (Z)-1⁶-chloro-6¹-isopropyl-2¹-(4-methoxybenzyl)-1¹,2⁵-dimethyl-1¹H,2¹H,6¹H-10-oxa-4,8-dithia-1(7,3)-indola-2(4,3),6(3,5)-dipyrazola-9(3,1)-naphthalenacyclotridecaphane-1²-carboxylic acid (8A, 13 mg, 0.016 mmol, 65%) as an off-white solid. MS (ESI) m/z 806.54 [M+1]⁺. ¹H NMR (400 MHz, DMSO-d₆) δ 13.5 (br s, 1H), 8.07 (d, J=7.6 Hz, 1H), 7.90 (d, J=8.8 Hz, 1H), 7.70 (d, J=7.6 Hz, 1H), 7.50-7.40 (m, 2H), 7.33 (s, 1H), 7.14 (d, J=8.4 Hz, 1H), 7.08 (d, J=8.4 Hz, 2H), 6.91 (d, J=8.8 Hz, 2H), 6.77 (s, 1H), 5.26 (s, 2H), 4.73 (s, 1H), 4.65-4.55 (m, 1H), 4.30 (s, 2H), 4.20-4.15 (m, 1H), 3.95-3.85 (m, 1H), 3.73 (s, 3H), 3.50-3.33 (m, 5H), 3.25-3.18 (m, 2H), 3.10-2.90 (m, 2H), 2.40-2.30 (m, 1H), 2.30-2.15 (m, 2H), 1.89 (s, 3H), 1.40-1.30 (m, 6H). LCMS purity: 99.00% and Chiral purity: 98.78%.

To a stirred solution of methyl (Z)-1⁶-chloro-6¹-isopropyl-2¹-(4-methoxybenzyl)-1¹,2⁵-dimethyl-1¹H,2¹H,6¹H-10-oxa-4,8-dithia-1(7,3)-indola-2(4,3),6(3,5)-dipyrazola-9(3,1)-naphthalenacyclotridecaphane-1²-carboxylate (8-1-peak-2, 21 mg, 0.0256 mmol) in degassed MeOH:THF:H₂O (1:1:1, 3 mL) was added LiOH.H₂O (16 mg, 0.384 mmol) at rt, and the mixture was heated at 60° C. for 1 h. The mixture was concentrated under reduced pressure, and the residue was acidified to pH-3 using 0.5 N aqueous HCl. The solid formed was filtered, and washed with water and pentane to afford (Z)-1⁶-chloro-6¹-isopropyl-2¹-(4-methoxybenzyl)-1¹,2⁵-dimethyl-1¹H,2¹H,6¹H-10-oxa-4,8-dithia-1(7,3)-indola-2(4,3),6(3,5)-dipyrazola-9(3,1)-naphthalenacyclotridecaphane-1²-carboxylic acid (8B, 14 mg, 0.017 mmol, 70%) as an off-white solid. MS (ESI) m/z 806.58 [M+1]⁺. ¹H NMR (400 MHz, DMSO-d₆) δ 13.30 (br s, 1H), 8.07 (d, J=8.4 Hz, 1H), 7.91 (d, J=8.8 Hz, 1H), 7.70 (d, J=7.2 Hz, 1H), 7.50-7.40 (m, 2H), 7.33 (s, 1H), 7.14 (d, J=7.6 Hz, 1H), 7.08 (d, J=8.8 Hz, 2H), 6.91 (d, J=8.8 Hz, 2H), 6.77 (s, 1H), 5.26 (s, 2H), 4.71 (s, 1H), 4.65-4.55 (m, 1H), 4.30 (s, 2H), 4.20-4.15 (m, 1H), 3.95-3.85 (m, 1H), 3.73 (s, 3H), 3.50-3.40 (m, 5H), 3.25-3.18 (m, 2H), 3.10-2.90 (m, 2H), 2.40-2.30 (m, 1H), 2.30-2.15 (m, 2H), 1.89 (s, 3H), 1.40-1.30 (m, 6H). LCMS purity: 98.70% and Chiral purity: 98.24%.

The absolute stereochemistry of compounds (8A) and (8B) is arbitrarily assigned.

### Example 9

### (Z)-1⁶-Chloro-6¹-isopropyl-1¹,2⁵-dimethyl-1¹H,2¹H,6¹H-10-oxa-4,8-dithia-1(7,3)-indola-2(4,3),6(3,5)-dipyrazola-9(3,1)-naphthalenacyclotridecaphane-1²-carboxylic acid

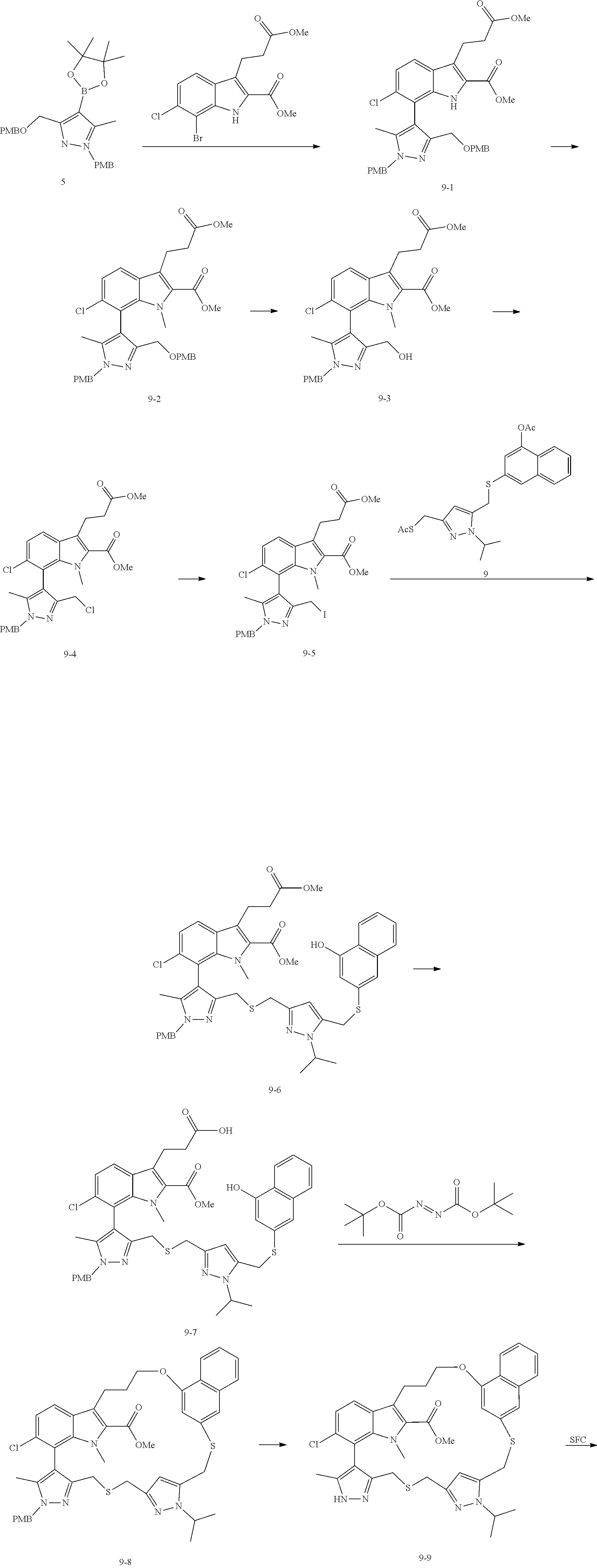

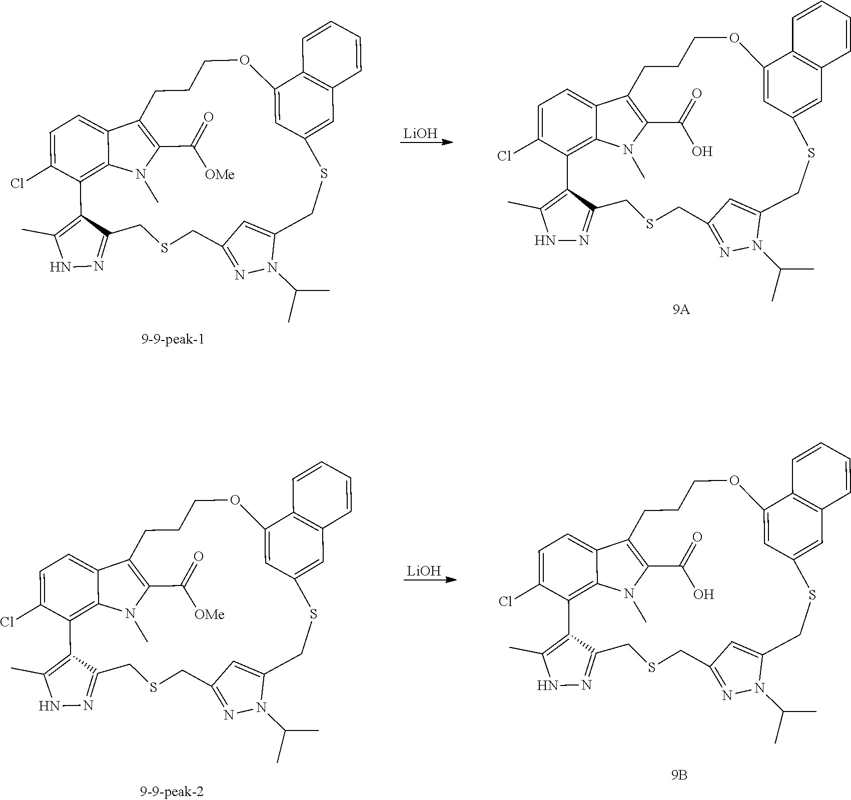

To the degassed solution of 1-(4-methoxybenzyl)-3-(((4-methoxybenzyl)oxy)methyl)-5-methyl-4-(4,4,5,5-tetramethyl-1,3,2-dioxaborolan-2-yl)-1H-pyrazole (7 g, 14.64 mmol) in 1,4-dioxane:water (56 mL: 14 mL) were added methyl 7-bromo-6-chloro-3-(3-methoxy-3-oxopropyl)-1H-indole-2-carboxylate (5.460 g, 14.64 mmol) and Cs₂CO₃ (9.516 g, 29.28 mmol). The solution was degassed with Ar for 20 mins. Dichloro[1 1′-bis(di-tert-butylphosphino)ferrocene] palladium(II) (571 mg, 0.878 mmol) was added, and the mixture was degassed again for 10 min. The mixture was heated at 100° C. for 2 h. The mixture was diluted with water (200 mL) and extracted with EtOAc (3×200 mL). The combined organic layers were dried over anhydrous Na₂SO₄, filtered and concentrated under reduced pressure to obtain a compound that was purified by silica gel column chromatography eluting using 30% EtOAc in PE to afford methyl 6-chloro-3-(3-methoxy-3-oxopropyl)-7-(1-(4-methoxybenzyl)-3-(((4-methoxybenzyl) oxy)methyl)-5-methyl-1H-pyrazol-4-yl)-1H-indole-2-carboxylate (9-1, 7.5 g, 11.62 mmol, 79% over 2 steps) as a brown viscous liquid. MS (ESI) m/z 646.34 [M+1]⁺. ¹H NMR (400 MHz, CDCl₃) δ 9.30 (s, 1H), 7.63 (d, J=8.8 Hz, 1H), 7.26-7.23 (m, 2H), 7.11 (d, J=8.8 Hz, 2H), 7.01-6.98 (m, 2H), 6.90-6.86 (m 2H), 6.76-6.73 (m, 2H), 5.41-5.25 (m, 2H), 4.44-4.36 (m, 3H), 4.18 (d, J=11.2 Hz, 1H), 3.80 (s, 3H), 3.78 (s, 3H), 3.73 (s, 3H), 3.66 (s, 3H), 3.44-3.35 (m, 2H), 2.72 (t, J=7.8 Hz, 2H), 2.02 (s, 3H).

To a stirred solution of methyl 6-chloro-3-(3-methoxy-3-oxopropyl)-7-(1-(4-methoxybenzyl)-3-(((4-methoxybenzyl)oxy)methyl)-5-methyl-1H-pyrazol-4-yl)-1H-indole-2-carboxylate (7.5 g, 11.62 mmol) in dry DMF (75 mL) were added Cs₂CO₃ (4.534 g, 13.953 mmol) and Mel (1.448 mL, 23.255 mmol), and the mixture was stirred at rt for 2.5 h. The reaction was quenched with water (250 mL) and extracted with EtOAc (3×500 mL). The organic layer was separated, dried over anhydrous Na₂SO₄, filtered and evaporated to afford methyl 6-chloro-3-(3-methoxy-3-oxopropyl)-7-(1-(4-methoxybenzyl)-3-(((4-methoxybenzyl)oxy)methyl)-5-methyl-1H-pyrazol-4-yl)-1-methyl-1H-indole-2-carboxylate (9-2, 7.5 g, 11.3 mmol, 97%) as a yellow liquid that was used without further purification. MS (ESI) m/z 660.61 [M+1]⁺. ¹H NMR (400 MHz, CDCl₃) δ 7.62 (d, J=8.8 Hz, 1H), 7.26-7.22 (m, 1H), 7.08 (d, J=8.4 Hz, 2H), 6.84-6.80 (m, 4H), 6.69-6.64 (m, 2H), 5.40-5.28 (m, 2H), 4.40-4.30 (m, 3H), 4.21 (d, J=11.6 Hz, 1H), 3.90 (s, 3H), 3.80 (s, 3H), 3.75 (s, 3H), 3.67 (s, 3H), 3.55 (br s, 1H), 3.48 (s, 3H), 3.40-3.30 (m, 2H), 2.66 (t, J=7.8 Hz, 2H), 1.95 (s, 2H).

To a stirred solution of methyl 6-chloro-3-(3-methoxy-3-oxopropyl)-7-(1-(4-methoxybenzyl)-3-(((4-methoxybenzyl)oxy)methyl)-5-methyl-1H-pyrazol-4-yl)-1-methyl-1H-indole-2-carboxylate (8.5 g, 12.898 mmol) in DCM (130 mL) was added TFA (7 mL, 90.28 mmol) at 0° C., and the mixture was stirred at rt for 2 h. The mixture was diluted with DCM (200 mL), washed with a sat. aq. NaHCO₃ solution (2×100 mL) and brine (2×100 mL). The organic layer was separated, dried over anhydrous Na₂SO₄, filtered and evaporated to give semi pure compound (8.4 g). The semi pure compound (8.4 g) was dissolved in MeOH (78 mL) and K₂CO₃ (3.39 g, 24.566 mmol) was added rt. The mixture was stirred for 2 h. The mixture was diluted with CH₂Cl₂ (200 mL), washed with water (2×100 mL), dried over anhydrous Na₂SO₄, filtered and concentrated under reduced pressure to afford the crude compound that was purified by silica gel column chromatography eluting at 30% EtOAc in PE to afford methyl 6-chloro-7-(3-(hydroxymethyl)-1-(4-methoxybenzyl)-5-methyl-1H-pyrazol-4-yl)-3-(3-methoxy-3-oxopropyl)-1-methyl-1H-indole-2-carboxylate (9-3, 4.0 g, 7.42 mmol, 57%) as a yellow liquid. MS (ESI) m/z 540.26 [M+1]⁺. ¹H NMR (400 MHz, CDCl₃) δ 7.63 (d, J=8.8 Hz, 1H), 7.26-7.24 (m, 1H), 7.08 (d, J=8.8 Hz, 2H), 6.88-6.85 (m, 2H), 5.38-5.26 (m, 2H), 4.52-4.48 (m, 2H), 3.92 (s, 3H), 3.80 (s, 3H), 3.67 (s, 3H), 3.51 (s, 3H), 3.35-3.30 (m, 2H), 2.65 (t, J=7.8 Hz, 2H), 1.95 (s, 3H), 1.86 (br s, 1H).

To a stirred solution of methyl 6-chloro-7-(3-(hydroxymethyl)-1-(4-methoxybenzyl)-5-methyl-1H-pyrazol-4-yl)-3-(3-methoxy-3-oxopropyl)-1-methyl-1H-indole-2-carboxylate (2.4 g, 4.44 mmol) in DCM (24 mL) under Ar was added SOCl₂ (0.48 mL, 6.66 mmol) at 0° C., and the mixture was stirred at rt for 30 min. The mixture was diluted with DCM (100 mL) and washed with a sat. NaHCO₃ solution (2×50 mL). The organic layer was separated, dried over anhydrous Na₂SO₄, filtered and evaporated to afford semi pure methyl 6-chloro-7-(3-(chloromethyl)-1-(4-methoxybenzyl)-5-methyl-1H-pyrazol-4-yl)-3-(3-methoxy-3-oxopropyl)-1-methyl-1H-indole-2-carboxylate (9-4, 2.38 g, 4.272 mmol, LCMS; 95%) as a light yellow liquid that was used without further purification. MS (ESI) m/z 558.54 [M+1]⁺.

To a stirred solution of semi pure methyl 6-chloro-7-(3-(chloromethyl)-1-(4-methoxybenzyl)-5-methyl-1H-pyrazol-4-yl)-3-(3-methoxy-3-oxopropyl)-1-methyl-1H-indole-2-carboxylate (2.38 g, 4.272 mmol) in dry MeCN (25 mL) were added NaI (1.15 g, 7.689 mmol) at rt, and the mixture was heated to 90° C. for 2.5 h. The solvent was evaporated, and the mixture was diluted with water (200 mL) and extracted with EtOAc (2×200 mL). The organic layer was separated, dried over Na₂SO₄, filtered and evaporated to afford semi pure methyl 6-chloro-7-(3-(iodomethyl)-1-(4-methoxybenzyl)-5-methyl-1H-pyrazol-4-yl)-3-(3-methoxy-3-oxopropyl)-1-methyl-1H-indole-2-carboxylate (9-5, 2.7 g, 4.160 mmol, LCMS: 93%) as a light yellow liquid that was used without further purification. MS (ESI) m/z 650.45 [M+1]⁺.

To a stirred solution of semi pure methyl 6-chloro-7-(3-(iodomethyl)-1-(4-methoxybenzyl)-5-methyl-1H-pyrazol-4-yl)-3-(3-methoxy-3-oxopropyl)-1-methyl-1H-indole-2-carboxylate (2.7 g, 4.16 mmol) in degassed MeOH (27 mL) were added K₂CO₃ (1.377 g, 9.96 mmol), and the mixture was degassed with Ar for 10 min. In another round bottom flask, 3-(((3-((acetylthio)methyl)-1-isopropyl-1H-pyrazol-5-yl)methyl)thio)naphthalen-1-yl acetate (1.95 g, 4.56 mmol) in methanol (20 mL) was degassed with Ar for 10 min, and this solution was added to previous mixture dropwise. The mixture was stirred at rt for 16 h. The mixture was diluted with CH₂Cl₂ (300 mL), and washed with water (100 mL) and brine (100 mL). The separated organic layer was dried over Na₂SO₄, filtered and evaporated to give a semi pure compound that was purified by silica gel column chromatography using 33% EtOAc in PE to afford methyl 6-chloro-7-(3-((((5-(((4-hydroxynaphthalen-2-yl)thio)methyl)-1-isopropyl-1H-pyrazol-3-yl)methyl)thio)methyl)-1-(4-methoxybenzyl)-5-methyl-1H-pyrazol-4-yl)-3-(3-methoxy-3-oxopropyl)-1-methyl-1H-indole-2-carboxylate (9-6, 1.9 g, 2.196 mmol, 49% over three steps) as an off-white solid. MS (ESI) m/z 866.52 [M+H]⁺.

To a stirred solution of methyl 6-chloro-7-(3-((((5-(((4-hydroxynaphthalen-2-yl)thio)methyl)-1-isopropyl-1H-pyrazol-3-yl)methyl)thio)methyl)-1-(4-methoxybenzyl)-5-methyl-1H-pyrazol-4-yl)-3-(3-methoxy-3-oxopropyl)-1-methyl-1H-indole-2-carboxylate (1.9 g, 2.196 mmol) in THF (20 mL) was added BH₃.THF (1M in THF) (10.9 mL, 10.98 mmol) at 0° C., and the mixture was stirred at rt for 3 h. The mixture was concentrated under reduced pressure, and the reaction was quenched with MeOH (5 mL) and aqueous 2N HCl (10 mL). The mixture was stirred at 0° C. stirred at rt for 30 min. The solution was extracted with CH₂Cl₂ (2×100 mL). The organic layer was washed with aq. NaHCO₃ solution, dried over anhydrous Na₂SO₄, filtered and concentrated to give a semi pure compound that was purified by silica gel column chromatography using 40% EtOAc in PE to afford methyl 6-chloro-7-(3-((((5-(((4-hydroxynaphthalen-2-yl)thio)methyl)-1-isopropyl-1H-pyrazol-3-yl)methyl)thio)methyl)-1-(4-methoxybenzyl)-5-methyl-1H-pyrazol-4-yl)-3-(3-hydroxypropyl)-1-methyl-1H-indole-2-carboxylate (9-7, 1.5 g, 1.792 mmol, 81%) as an off-white solid. MS (ESI) m/z 838.64 [M+1]⁺. ¹H NMR (400 MHz, DMSO-d₆) δ 8.03 (d, J=8.0 Hz, 1H), 7.72-7.68 (m, 2H), 7.44-7.35 (m, 2H), 7.31 (br s, 1H), 7.25 (d, J=8.8 Hz, 1H), 7.11 (d, J=8.8 Hz, 2H), 6.90 (d, J=8.4 Hz, 2H), 6.79 (s, 1H), 5.90 (s, 1H), 5.30 (s, 2H), 4.60-4.40 (m, 2H), 4.28 (s, 2H), 3.82 (s, 3H), 3.73 (s, 3H), 3.50-3.33 (m, 9H), 3.05-2.95 (m, 1H), 1.99 (s, 1H), 1.89 (s, 3H), 1.75-1.65 (m, 2H), 1.28 (d, J=6.4 Hz, 6H).

A solution of TPP (1.1 g, 4.4 mmol) in pre-degassed toluene (15 mL) was stirred at 90° C. for 10 min. To this a solution of methyl 6-chloro-7-(3-((((5-(((4-hydroxynaphthalen-2-yl)thio)methyl)-1-isopropyl-1H-pyrazol-3-yl)methyl)thio)methyl)-1-(4-methoxybenzyl)-5-methyl-1H-pyrazol-4-yl)-3-(3-hydroxypropyl)-1-methyl-1H-indole-2-carboxylate (1.5 g, 2.0 mmol) and di-tert-butyl (E)-diazene-1,2-dicarboxylate (1.0 g, 4.4 mmol) in pre-degassed THF (10.0 mL) as added dropwise at 90° C. The mixture was stirred under N₂ for 2 h. The reaction was quenched with water (40 mL) and extracted with EtOAc (3×25 mL). The organic layer was collected, dried over Na₂SO₄, filtered and evaporated to give semi pure 9-8 that was purified by silica gel column chromatography using 0% to 40% EtOAc in PE to afford methyl (Z)-1⁶-chloro-6¹-isopropyl-2¹-(4-methoxybenzyl)-1¹,2⁵-dimethyl-1¹H,2¹H,6¹H-10-oxa-4,8-dithia-1(7,3)-indola-2(4,3),6(3,5)-dipyrazola-9(3,1)-naphthalenacyclotridecaphane-1²-carboxylate (9-8, 1 g, 1.2 mmol, 60%) as a light yellow solid. MS (LCMS) m/z 820.52 [M+H]⁺. ¹H NMR (400 MHz, CDCl₃) δ 8.30-8.25 (m, 1H), 7.70-7.65 (m, 2H), 7.55 (d, J=8.8 Hz, 2H), 7.51-7.45 (m, 3H), 7.02 (dd, J=8.6 Hz, and 14.0 Hz, 3H), 6.84 (d, J=8.8 Hz, 2H), 6.34 (s, 1H), 5.30-5.20 (m, 2H), 4.91 (s, 1H), 4.50-4.40 (m, 1H), 4.0-3.90 (m, 2H), 3.88 (s, 3H), 3.35-3.20 (m, 5H), 3.61 (s, 3H), 3.60-3.45 (m, 2H), 3.35-3.30 (m, 1H), 3.25-3.15 (m, 2H), 2.78 (d, J=13.6 Hz, 1H), 2.50-2.40 (m, 1H), 2.30-2.20 (m, 1H), 1.91 (s, 3H), 1.49 (d, J=6.4 Hz, 3H), 1.41 (d, J=6.8 Hz, 3H).

To a stirred solution of methyl (Z)-1⁶-chloro-6¹-isopropyl-2¹-(4-methoxybenzyl)-1¹,2⁵-dimethyl-1¹H,2¹H,6¹H-10-oxa-4,8-dithia-1(7,3)-indola-2(4,3),6(3,5)-dipyrazola-9(3,1)-naphthalenacyclotridecaphane-1²-carboxylate (1.0 g, 1.2 mmol) was added in TFA (10.0 mL) and anisole (1.3 mL, 12.1 mmol) at ambient temperature. The mixture was stirred at 100° C. for 48 h. The mixture was concentrated and diluted with water (30 mL) and extracted with EtOAc (2×30 mL). The organic layer was washed with a sat. NaHCO₃ solution (3×10 mL). The organic layer was separated, dried over Na₂SO₄, filtered and evaporated to afford a crude that was purified by silica gel column chromatography using 0% to 60% EtOAc in PE to afford methyl (Z)-1⁶-chloro-6¹-isopropyl-1¹,2⁵-dimethyl-1¹H,2¹H,6¹H-10-oxa-4,8-dithia-1(7,3)-indola-2(4,3),6(3,5)-dipyrazola-9(3,1)-naphthalenacyclotridecaphane-1²-carboxylate (9-9, 650 mg, 0.92 mmol) as a light yellow solid. MS (LCMS) m/z 700.42 [M+H]⁺. ¹H NMR (400 MHz, CDCl₃) δ 8.20-8.14 (m, 1H), 7.70-7.65 (m, 1H), 7.58 (d, J=8.8 Hz, 1H), 7.50-7.45 (m, 2H), 7.31 (s, 1H), 7.01 (d, J=8.8 Hz, 1H), 6.36 (s, 1H), 4.83 (s, 1H), 4.55-4.45 (m, 1H), 3.95-3.80 (m, 7H), 3.59 (s, 3H), 3.59-3.50 (m, 2H), 3.25-3.15 (m, 3H), 3.02 (d, J=14.0 Hz, 1H), 2.92 (d, J=14.0 Hz, 1H), 2.50-2.40 (m, 1H), 2.35-2.20 (m, 1H), 2.06 (s, 3H), 1.50 (d, J=6.4 Hz, 3H), 1.40 (d, J=6.4 Hz, 3H).

Methyl (Z)-1⁶-chloro-6¹-isopropyl-1¹,2⁵-dimethyl-1¹H,2¹H,6¹H-10-oxa-4,8-dithia-1(7,3)-indola-2(4,3),6(3,5)-dipyrazola-9(3,1)-naphthalenacyclotridecaphane-1²-carboxylate (9-9, 300 mg) was purified by SFC purification to afford 9-9-peak-1 (130 mg) as an off white solid and 9-9-peak-2 (100 mg) as an off white solid. These two peaks were separately used for the next steps to get respective final compound.

9-9-peak-1: MS (LCMS) m/z 700.40 [M+H]⁺. ¹H NMR (400 MHz, CDCl₃) δ 9.90 (s, 1H), 8.13 (br s, 1H), 7.65 (br s, 1H), 7.58 (d, J=8.8 Hz, 2H), 7.50-7.45 (m, 2H), 7.01 (d, J=8.8 Hz, 1H), 6.36 (s, 1H), 4.83 (br s, 1H), 4.55-4.45 (m, 1H), 3.95-3.80 (m, 8H), 3.67-3.50 (m, 4H), 3.25-3.15 (m, 2H), 2.91 (br s, 2H), 2.44 (br s, 1H), 2.30 (bs, 1H), 2.06 (s, 3H), 1.50 (d, J=6.4 Hz, 3H), 1.41 (d, J=6.4 Hz, 3H); LCMS purity: 97.53; Chiral purity: 99.96%.

9-9-peak-2: MS (LCMS) m/z 700.88 [M+H]⁺. ¹H NMR (400 MHz, CDCl₃) δ 9.81 (br s, 1H), 8.13 (br s, 1H), 7.66 (br s, 2H), 7.58 (d, J=8.8 Hz, 2H), 7.47-7.45 (m, 2H), 7.01 (d, J=8.8 Hz 1H), 6.36 (s, 1H), 4.82 (br s, 1H), 4.52-4.45 (m, 1H), 4.00-3.80 (m, 7H), 3.67-3.50 (m, 4H), 3.23-3.16 (m, 2H), 2.91 (bs, 2H), 2.45 (br s, 1H), 2.35 (bs, 1H), 2.06 (s, 3H), 1.49 (m, 3H), 1.41 (d, J=5.6 Hz, 3H); LCMS purity: 95.55%; Chiral purity: 99.84%.

To a stirred solution of (Z)-1⁶-chloro-6¹-isopropyl-1¹,2⁵-dimethyl-1¹H,2¹H,6¹H-10-oxa-4,8-dithia-1(7,3)-indola-2(4,3),6(3,5)-dipyrazola-9(3,1)-naphthalenacyclotridecaphane-1²-carboxylate (9-9-peak-1, 130 mg, 0.034 mmol) in MeOH:THF:H₂O (1:1:1 12 mL) was added a solution of LiOH.H₂O (117 mg, 2.78 mmol) at rt, and the mixture was stirred at 70° C. for 2 h. The volatiles were removed by evaporation. The aqueous layer was acidified to pH 2 using 6 N aqueous HCl, and an off white precipitate was formed. The mixture was filtered, and the solid and washed with ice cold water, dried in vacuum to give (Z)-1⁶-chloro-6¹-isopropyl-1¹,2⁵-dimethyl-1¹H,2¹H,6¹H-10-oxa-4,8-dithia-1(7,3)-indola-2(4,3),6(3,5)-dipyrazola-9(3,1)-naphthalenacyclotridecaphane-1²-carboxylic acid (9A, 110 mg, 0.16 mmol, 86%) as an off white solid. MS (LCMS) m/z 686.52 [M+H]⁺. ¹H NMR (400 MHz, DMSO-d₆) δ 12.8 (br s, 1H), 8.07 (d, J=8.0 Hz, 1H), 7.84 (d, J=8.4 Hz, 1H), 7.70 (d, J=7.4 Hz, 1H), 7.49-7.42 (m, 2H), 7.33 (s, 1H), 7.10 (d, J=8.4 Hz, 1H), 6.75 (s, 1H), 4.76 (s, 1H), 4.64-4.55 (m, 1H), 4.34-4.23 (m, 2H), 4.13 (d, J=8.4 Hz, 1H), 3.95-3.85 (m, 1H), 3.46-3.41 (m, 6H), 3.20-3.13 (m, 2H), 3.10-2.90 (m, 2H), 2.40-2.15 (m, 2H), 1.90 (s, 3H), 1.34-1.29 (m, 6H). LCMS purity: 97.69%; HPLC purity: 97.42%; Chiral purity: 99.83%.

To a stirred solution (Z)-1⁶-chloro-6¹-isopropyl-1¹,2⁵-dimethyl-1¹H,2¹H,6¹H-10-oxa-4,8-dithia-1(7,3)-indola-2(4,3),6(3,5)-dipyrazola-9(3,1)-naphthalenacyclotridecaphane-1²-carboxylate (9-9-peak-2, 100 mg, 0.143 mmol) in MeOH:THF:H₂O (1:1:lv/v/v 12 mL) was added LiOH.H₂O (90 mg, 2.14 mmol) at rt. The mixture was stirred at 70° C. for 2 h. The solvent was evaporated, and the aqueous layer was acidified to pH 2 using 6 N aq. HCl. The white precipitate was filtered off, washed with ice-water, dried in vacuum to afford (Z)-1⁶-chloro-6¹-isopropyl-1¹,2⁵-dimethyl-1¹H,2¹H,6¹H-10-oxa-4,8-dithia-1(7,3)-indola-2(4,3),6(3,5)-dipyrazola-9(3,1)-naphthalenacyclo tridecaphane-1²-carboxylic acid (9B, 90 mg, 0.13 mmol, 92%) as an off white solid. MS (LCMS) m/z 686.52 [M+H]⁺. ¹H NMR (400 MHz, DMSO-d₆) δ 13.2 (bs, 1H), 8.08 (d, J=7.6 Hz, 1H), 7.89 (d, J=8.4 Hz, 1H), 7.70 (d, J=7.2 Hz, 1H), 7.50-7.40 (m, 2H), 7.34 (s, 1H), 7.12 (d, J=8.4 Hz, 1H), 6.73 (s, 1H), 4.69 (s, 1H), 4.65-4.55 (m, 1H), 4.27 (s, 2H), 4.15-4.10 (m, 1H), 3.95-3.85 (m, 1H), 3.44-3.41 (m, 6H), 3.19-3.11 (m, 3H), 2.96-2.93 (m, 1H), 2.42-2.30 (m, 1H), 2.30-2.15 (m, 1H), 1.93 (s, 3H), 1.34-1.29 (m, 6H). LCMS purity: 98.01%; HPLC purity: 97.59%; Chiral purity: 99.89%.

The absolute stereochemistry of compounds (9A) and (9B) is arbitrarily assigned.

### Example 10

### (Z)-1⁶-Chloro-6¹-isopropyl-1¹,2¹,2⁵-trimethyl-1¹H,2¹H,6¹H-10-oxa-4,8-dithia-1(7,3)-indola-2(4,3),6(3,5)-dipyrazola-9(3,1)-naphthalenacyclotridecaphane-1²-carboxylic acid

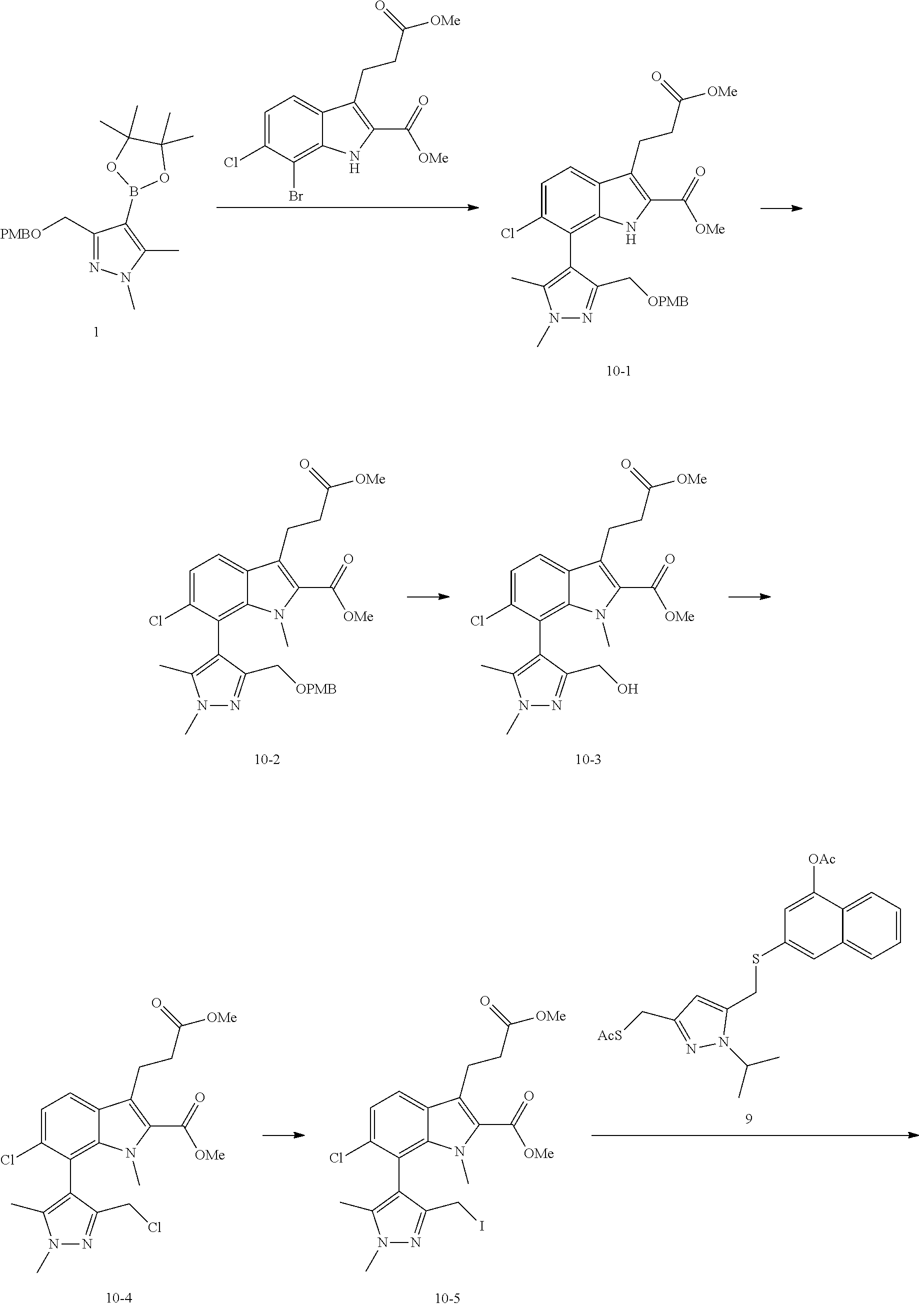

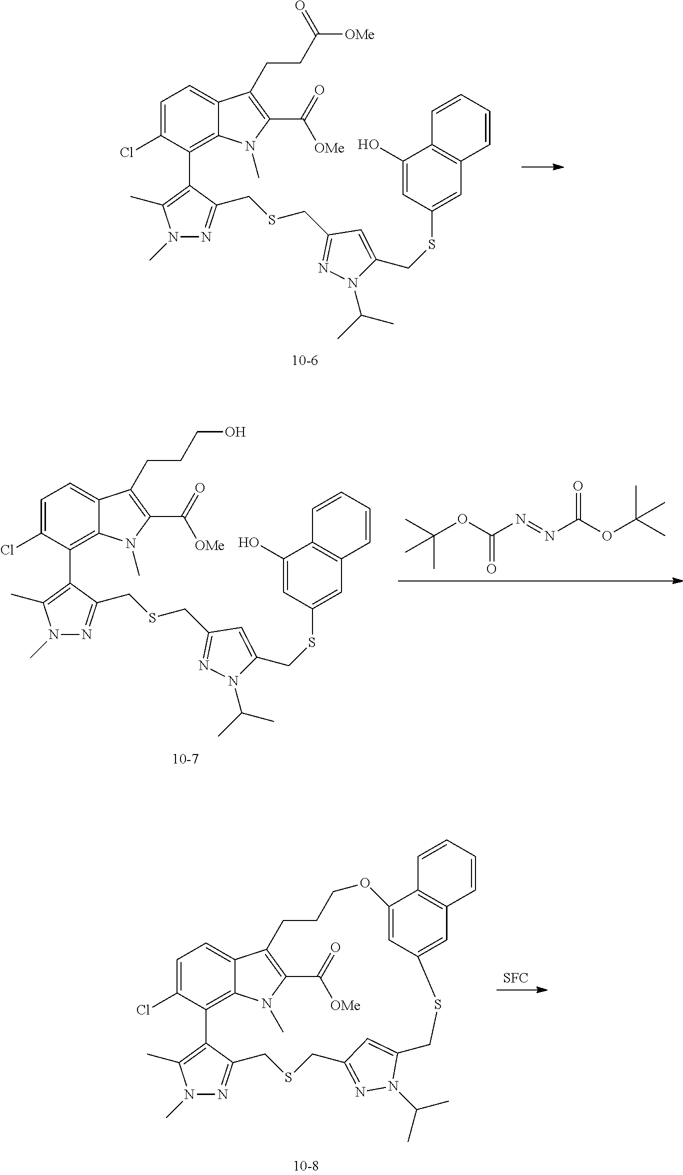

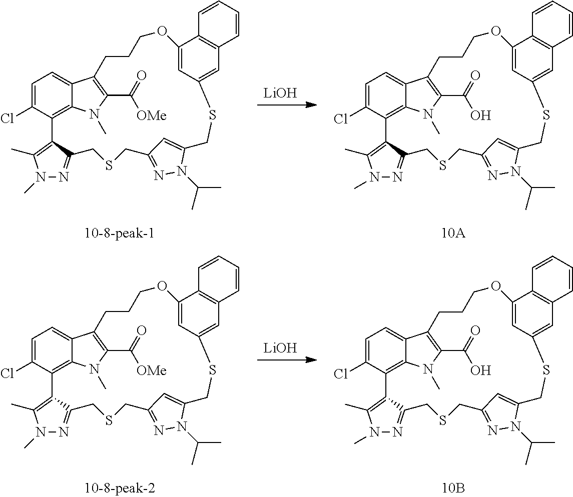

To the degassed solution of 3-(((4-methoxybenzyl)oxy)methyl)-1,5-dimethyl-4-(4,4,5,5-tetramethyl-1,3,2-dioxaborolan-2-yl)-1H-pyrazole (1) (2×6.04 g, 16.20 mmol) in 1,4-dioxane:water (50 mL:12.5 mL) were added methyl 7-bromo-6-chloro-3-(3-methoxy-3-oxopropyl)-1H-indole-2-carboxylate (4.85 g, 12.96 mmol) and Cs₂CO₃ (8.42 g, 25.93 mmol). The solution was degassed with Ar for 15 min. followed by THE addition of dichloro[1 1′-bis(di-tert-butylphosphino)ferrocene] palladium(II) (253 mg, 0.3 mmol). The solution was degassed again for 15 mins. The mixture was heated at 100° C. for 3 h. After consumption of starting material, the reaction was mixed, diluted with water (500 mL) and extracted with EtOAc (3×500 mL). The organic layer was dried over Na₂SO₄, filtered and evaporated to give semi pure 7. The crude compound was purified by silica gel column chromatography using 50% EtOAc in PE to afford methyl 6-chloro-3-(3-methoxy-3-oxopropyl)-7-(3-(((4-methoxybenzyl)oxy)methyl)-1,5-dimethyl-1H-pyrazol-4-yl)-1H-indole-2-carboxylate (10-1, 9 g, 16.66 mmol, 64%) as a brown liquid. MS (LCMS) m/z 540.15 [M+H]⁺.

To a stirred solution of methyl 6-chloro-3-(3-methoxy-3-oxopropyl)-7-(3-(((4-methoxybenzyl)oxy)methyl)-1,5-dimethyl-1H-pyrazol-4-yl)-1H-indole-2-carboxylate (2×6 g, 11.110 mmol) in dry DMF (70 mL) were added Cs₂CO₃ (5.416 g, 16.666 mmol) and Mel (0.8 mL, 12.222 mmol), and the mixture was stirred at rt for 3 h. The reaction was quenched with water (150 mL) and extracted with EtOAc (3×500 mL). The organic layer was separated, dried over Na₂SO₄, filtered and evaporated to afford methyl 6-chloro-3-(3-methoxy-3-oxopropyl)-7-(3-(((4-methoxybenzyl)oxy)methyl)-1,5-dimethyl-1H-pyrazol-4-yl)-1-methyl-1H-indole-2-carboxylate (10-2, 12.5 g, 22.561 mmol) as a brown semi solid that was used without further purification. MS (LCMS) m/z 554.87 [M+H]⁺.

To a stirred solution of methyl 6-chloro-3-(3-methoxy-3-oxopropyl)-7-(3-(((4-methoxybenzyl)oxy)methyl)-1,5-dimethyl-1H-pyrazol-4-yl)-1-methyl-1H-indole-2-carboxylate (2×6 g, 10.829 mmol) in DCM (90 mL) was added TFA (14 mL, 182.29 mmol) at 0° C., and the mixture was stirred at rt for 1.5 h. The mixture was diluted with DCM (500 mL), washed with a sat. aq. NaHCO₃ solution (3×150 mL). The organic layer was separated, dried over Na₂SO₄, filtered and evaporated to give semi pure 10-3 that was purified by silica gel column chromatography using 5% MeOH in DCM to afford to afford methyl 6-chloro-7-(3-(hydroxymethyl)-1,5-dimethyl-1H-pyrazol-4-yl)-3-(3-methoxy-3-oxopropyl)-1-methyl-1H-indole-2-carboxylate (10-3, 5.8 g, 13.367 mmol, 60% for two steps) as a brown semi solid. MS (LCMS) m/z 434.34 [M+H]⁺. ¹H NMR (400 MHz, CDCl₃) δ 7.63 (d, J=8.8 Hz, 2H), 7.25 (d, J=7.6 Hz, 2H), 3.92 (s, 3H), 3.89 (s, 3H), 3.68 (s, 3H), 3.53 (s, 3H), 3.34 (t, J=7.6 Hz, 2H), 2.68-2.64 (m, 2H), 2.05-2.03 (m, 4H).

To a stirred solution of methyl 6-chloro-7-(3-(hydroxymethyl)-1,5-dimethyl-1H-pyrazol-4-yl)-3-(3-methoxy-3-oxopropyl)-1-methyl-1H-indole-2-carboxylate (5.5 g, 12.676 mmol) in DCM (70 mL) under Ar was added SOCl₂ (1.79 mL, 15.211 mmol) at 0° C., and the mixture was stirred at rt for 1 h. The mixture was diluted with DCM (250 mL) and washed with a sat. NaHCO₃ solution (3×100 mL). The organic layer was separated, dried over Na₂SO₄, filtered and evaporated to afford semi pure methyl 6-chloro-7-(3-(chloromethyl)-1,5-dimethyl-1H-pyrazol-4-yl)-3-(3-methoxy-3-oxopropyl)-1-methyl-1H-indole-2-carboxylate (10-4, 5.7 g, 12.601 mmol) as a brown semi solid that was used without further purification. MS (LCMS) m/z 452.06 [M+H]⁺.

To a stirred solution of semi pure methyl 6-chloro-7-(3-(chloromethyl)-1,5-dimethyl-1H-pyrazol-4-yl)-3-(3-methoxy-3-oxopropyl)-1-methyl-1H-indole-2-carboxylate (5.7 g, 12.601 mmol) in dry MeCN (70 mL) was added NaI (3.377 g, 22.682 mmol) at rt, and the mixture was heated to 80° C. for 2 h. The solvent was evaporated, and the mixture was diluted with water (150 mL) and extracted with EtOAc (3×300 mL). The organic layer was separated, dried over Na₂SO₄, filtered and evaporated to afford semi pure methyl 6-chloro-7-(3-(iodomethyl)-1,5-dimethyl-1H-pyrazol-4-yl)-3-(3-methoxy-3-oxopropyl)-1-methyl-1H-indole-2-carboxylate (10-5, 6.2 g, 11.401 mmol) as a brown semi solid that was used without further purification. MS (LCMS) m/z 544.28 [M+H]⁺.

To a stirred solution of semi pure methyl 6-chloro-7-(3-(iodomethyl)-1,5-dimethyl-1H-pyrazol-4-yl)-3-(3-methoxy-3-oxopropyl)-1-methyl-1H-indole-2-carboxylate (2×3 g, 5.516 mmol) in MeOH (30 mL) was added K₂CO₃ (1.8 g, 13.238 mmol), and the solution was degassed with Ar for 10 min. In another round bottom flask, 3-(((3-((acetylthio)methyl)-1-isopropyl-1H-pyrazol-5-yl)methyl)thio)naphthalen-1-yl acetate (C) (2.6 g, 6.068 mmol) in methanol (15 mL) was degassed with Ar for 10 min, and this solution was added to previous mixture dropwise. The mixture was stirred at rt for 1 h. The solvent was evaporated, and the mixture was diluted with water (200 mL) and extracted with EtOAc (2×500 mL). The organic layer was separated, dried over Na₂SO₄, filtered and evaporated to get semi pure 10-5 that was purified by silica gel column chromatography using 3% MeOH in DCM to afford methyl 6-chloro-7-(3-((((5-(((4-hydroxynaphthalen-2-yl)thio)methyl)-1-isopropyl-1H-pyrazol-3-yl)methyl)thio)methyl)-1,5-dimethyl-1H-pyrazol-4-yl)-3-(3-methoxy-3-oxopropyl)-1-methyl-1H-indole-2-carboxylate (10-6, 3.4 g, 4.471 mmol, 32% for two steps) as a light brown solid. MS (LCMS) m/z 760.42 [M+H]⁺. ¹H NMR (400 MHz, CDCl₃) δ 9.91 (s, 1H), 8.27 (d, J=7.6 Hz, 1H), 7.76 (dd, J=6.8 Hz and 2.0 Hz, 1H), 7.66-7.60 (m, 2H), 7.52-7.45 (m, 2H), 7.42-7.21 (m, 2H, merged with solvent peak), 6.59 (s, 1H), 5.84 (s, 1H), 4.30-4.23 (m, 1H), 3.93-3.90 (m, 5H), 3.90-3.85 (m, 3H), 3.70 (s, 3H), 3.60-3.45 (m, 8H), 3.40-3.30 (m, 2H), 2.69 (t, J=8.0 Hz, 2H), 1.45-1.35 (m, 6H).

To a stirred solution of methyl 6-chloro-7-(3-((((5-(((4-hydroxynaphthalen-2-yl)thio)methyl)-1-isopropyl-1H-pyrazol-3-yl)methyl)thio)methyl)-1,5-dimethyl-1H-pyrazol-4-yl)-3-(3-methoxy-3-oxopropyl)-1-methyl-1H-indole-2-carboxylate (3.4 g, 4.471 mmol) in THF (40 mL) was added BH₃.THF (1M in THF) (27 mL, 26.829 mmol) at 0° C., and the mixture was stirred at rt for 6 h. The volatile were removed, and the reaction was quenched with MeOH (5 mL) and 6N HCl (10 mL). The mixture was stirred for 30 min and extracted with EtOAc (2×500 mL). The organic layer was dried over Na₂SO₄, filtered and concentrated to give semi pure 10-5 that was purified by silica gel column chromatography using 6% MeOH in DCM to afford methyl 6-chloro-7-(3-((((5-(((4-hydroxynaphthalen-2-yl)thio)methyl)-1-isopropyl-1H-pyrazol-3-yl)methyl)thio)methyl)-1,5-dimethyl-1H-pyrazol-4-yl)-3-(3-hydroxypropyl)-1-methyl-1H-indole-2-carboxylate (10-7, 1.7 g, 2.324 mmol, 54%) as a brown sticky solid. MS (LCMS) m/z 732. [M+H]⁺. ¹H NMR (400 MHz, CDCl₃) δ 9.90 (s, 1H), 8.28-8.26 (m, 1H), 7.77-7.74 (m, 1H), 7.64-7.61 (m, 2H), 7.51-7.45 (m, 2H), 7.26-7.24 (m, 2H, merged with solvent peak), 6.59 (s, 1H), 5.83 (s, 1H), 4.30-4.20 (m, 1H), 4.00-3.90 (m, 7H), 3.70-3.65 (m, 3H), 3.60-3.40 (m, 7H), 3.17 (t, J=7.2 Hz, 2H), 2.06 (s, 3H), 2.00-1.90 (m, 2H), 1.35-1.25 (m, 6H).

To a stirred solution of TPP (501 mg, 1.911 mmol) in toluene (10 mL) in was added a solution of di-tert-butyl diazene-1,2-dicarboxylate (440 mg, 1.911 mmol) and methyl 6-chloro-7-(3-((((5-(((6-chloro-4-hydroxynaphthalen-2-yl)thio)methyl)-1-methyl-1H-pyrazol-3-yl)methyl) thio)methyl)-1,5-dimethyl-1H-pyrazol-4-yl)-3-(3-hydroxypropyl)-1-methyl-1H-indole-2-carboxylate (2×700 mg, 0.955 mmol) in toluene (5 mL) and THF (2 mL) at 70° C., and the mixture was stirred at 70° C. for 16 h. The reaction was quenched with water (50 mL) and extracted with EtOAc (2×250 mL). The organic layer was dried over Na₂SO₄, filtered, concentrated to give the semi pure desired compound that was purified by silica gel column chromatography using 50% EtOAc to afford 10-8 together with some TPPO, which was triturated with MeOH (5 mL), to afford methyl (Z)-1⁶-chloro-6¹-isopropyl-1¹,2¹,2⁵-trimethyl-1¹H,2¹H,6¹H-10-oxa-4,8-dithia-1(7,3)-indola-2(4,3),6(3,5)-dipyrazola-9(3,1)-naphthalenacyclotridecaphane-1²-carboxylate (10-8, 180 mg, 0.245 mmol, 15%) as an off white sticky solid. MS (LCMS) m/z 714.45 [M+H]⁺. ¹H NMR (400 MHz, CDCl₃) δ 8.29-8.26 (m, 1H), 7.71-7.68 (m, 1H), 7.56-7.44 (m, 4H), 6.99 (d, J=8.4 Hz, 1H), 6.33 (s, 1H), 4.89 (s, 1H), 4.50-4.44 (m, 1H), 4.00-3.70 (m, 10H), 3.64 (s, 3H), 3.53-3.47 (m, 2H), 3.28-3.17 (dd, J=13.6 Hz and 14.0 Hz, 1H), 2.78 (d, J=13.6 Hz, 3H), 2.50-2.40 (m, 1H), 2.32-2.20 (m, 1H), 2.04 (s, 2H), 2.03 (s, 3H), 1.47 (d, J=6.4 Hz, 3H), 1.40 (d, J=6.4 Hz, 3H), 1.26 (t, J=6.8 Hz, 1H).

Methyl (Z)-1⁶-chloro-6¹-isopropyl-1¹,2¹,2⁵-trimethyl-1¹H,2¹H,6¹H-10-oxa-4,8-dithia-1(7,3)-indola-2(4,3),6(3,5)-dipyrazola-9(3,1)-naphthalenacyclotridecaphane-1²-carboxylate (170 mg) was purified by SFC purification to afford 10-8-peak-1 (60 mg) as an off white solid with 97.61% of LCMS purity (chiral HPLC:99.78%) and 10-8-peak-2 (60 mg) as an off white solid with 99.58% of LCMS purity (chiral HPLC:98.51%). These two peaks were separately used for the next steps to get respective final compounds.

10-8-peak-2: MS (LCMS) m/z 714.31 [M+H]⁺. ¹H NMR (400 MHz, CDCl₃) δ 8.29-8.26 (m, 1H), 7.70-7.68 (m, 1H), 7.51-7.46 (m, 3H), 6.99 (d, J=8.4 Hz, 1H), 6.33 (s, 1H), 4.89 (s, 1H), 4.49-4.44 (m, 1H), 4.00-3.70 (m, 11H), 3.64 (s, 2H), 3.53-3.47 (m, 2H), 3.28-3.17 (dd, J=13.6 Hz and 14.0 Hz, 3H), 2.78 (d, J=13.6 Hz, 1H), 2.50-2.40 (m, 1H), 2.32-2.20 (m, 1H), 2.02 (s, 3H), 1.47 (d, J=6.4 Hz, 3H), 1.40 (d, J=6.4 Hz, 3H), 1.26 (t, J=6.8 Hz, 1H).

10-8-peak-2: MS (LCMS) m/z 714.34 [M+H]⁺. ¹H NMR (400 MHz, CDCl₃) δ 8.29-8.26 (m, 1H), 7.71-7.68 (m, 1H), 7.51-7.46 (m, 3H), 6.99 (d, J=8.4 Hz, 1H), 6.33 (s, 1H), 4.89 (s, 1H), 4.50-4.44 (m, 1H), 4.00-3.70 (m, 11H), 3.64 (s, 2H), 3.53-3.47 (m, 2H), 3.28-3.17 (dd, J=13.6 Hz and 14.0 Hz, 3H), 2.78 (d, J=13.6 Hz, 1H), 2.50-2.40 (m, 1H), 2.32-2.20 (m, 1H), 2.02 (s, 3H), 1.47 (d, J=6.4 Hz, 3H), 1.40 (d, J=6.4 Hz, 3H), 1.26 (t, J=6.8 Hz, 1H).

To a stirred solution of methyl (Z)-1⁶-chloro-6¹-isopropyl-1¹,2¹,2⁵-trimethyl-1¹H,2¹H,6¹H-10-oxa-4,8-dithia-1(7,3)-indola-2(4,3),6(3,5)-dipyrazola-9(3,1)-naphthalenacyclotridecaphane-1²-carboxylate (10-8-peak-1, 60 mg, 0.084 mmol) in THF:H₂O (3:1, 3 mL) was added of LiOH.H₂O in 3 equal portions in 3 h intervals (52 mg, 1.26 mmol) at 0° C. The mixture was stirred at rt for 16 h. The solvent was evaporated, and the aqueous layer was acidified to pH 2 using 2 N aq. HCl. The mixture was diluted with water (15 mL) and extracted with EtOAc (2×50 mL). The organic layer was separated, dried over Na₂SO₄, filtered and evaporated to get semi pure 10A that was purified by prep-TLC to afford (Z)-1⁶-chloro-6¹-isopropyl-1¹,2¹,2⁵-trimethyl-1¹H,2¹H,6¹H-10-oxa-4,8-dithia-1(7,3)-indola-2(4,3),6(3,5)-dipyrazola-9(3,1)-naphthalenacyclotridecaphane-1²-carboxylic acid (10A, 14 mg, 0.020. mmol, 24%) as an off white solid. MS (LCMS) m/z 700.34 [M+H]⁺. ¹H NMR (400 MHz, DMSO-d₆) δ 8.05 (d, J=7.6 Hz, 1H), 7.80-7.75 (m, 1H), 7.69 (d, J=7.2 Hz, 1H), 7.50-7.40 (m, 2H), 7.30 (s, 1H), 7.08 (d, J=8.4 Hz, 1H), 6.82 (s, 1H), 4.90 (br s, 1H), 4.70-4.55 (m, 1H), 4.50-4.40 (m, 1H), 4.26-4.22 (m, 1H), 4.15-4.12 (m, 1H), 4.00-3.90 (m, 1H), 3.75 (s, 3H), 3.50-3.40 (m, 5H), 3.20-3.10 (m, 2H), 3.01-2.95 (m, 2H), 2.33-2.25 (m, 2H), 1.95 (s, 3H), 1.34-1.29 (m, 6H), 1.25 (s, 1H). HPLC: 95.14%; Chiral purity: 98.57%.

To a stirred solution of methyl (Z)-1⁶-chloro-6¹-isopropyl-1¹,2¹,2⁵-trimethyl-1¹H,2¹H,6¹H-10-oxa-4,8-dithia-1(7,3)-indola-2(4,3),6(3,5)-dipyrazola-9(3,1)-naphthalenacyclotridecaphane-1²-carboxylate (10-8-peak-2, 60 mg, 0.084 mmol) in THF:H₂O (3:1, 3 mL) was added of LiOH.H₂O in 3 equal portions in 3 h intervals (52 mg, 1.26 mmol) at 0° C., and the mixture was stirred at rt for 16 h. The solvent was evaporated, and the aqueous layer was acidified to pH 2 using 2 N aq. HCl. The mixture was diluted with water (15 mL) and extracted with EtOAc (2×50 mL). The organic layer was separated, dried over Na₂SO₄, filtered and evaporated to get semi pure 10B that was purified by prep-TLC to afford (Z)-1⁶-chloro-6¹-isopropyl-1¹,2¹,2⁵-trimethyl-1¹H,2¹H,6¹H-10-oxa-4,8-dithia-1(7,3)-indola-2(4,3),6(3,5)-dipyrazola-9(3,1)-naphthalenacyclotridecaphane-1²-carboxylic acid (10B, 21 mg, 0.028 mmol, 34%) as an off white solid. MS (LCMS) m/z 700.34 [M+H]⁺. ¹H NMR (400 MHz, DMSO-d₆) δ 8.05 (d, J=7.6 Hz, 1H), 7.90 (d, J=9.2 Hz, 1H), 7.70 (d, J=1.2 Hz, 1H), 7.50-7.40 (m, 2H), 7.33 (s, 1H), 7.14 (d, J=8.8 Hz, 1H), 6.76 (s, 1H), 4.70 (br s, 1H), 4.65-4.55 (m, 1H), 4.29 (s, 2H), 4.20-4.15 (m, 1H), 3.95-3.85 (m, 1H), 3.76 (s, 3H), 3.50-3.40 (m, 5H), 3.20-3.10 (m, 3H), 2.40-2.35 (m, 1H), 2.30-2.15 (m, 1H), 1.95 (s, 3H), 1.34-1.29 (m, 6H), 1.25 (s, 2H). HPLC: 94.59%; Chiral purity: 98.45%.

The absolute stereochemistry of compounds (10A) and (10B) is arbitrarily assigned.

### Example 11

### (Z)-1⁶-Chloro-1¹,2¹,2⁵-trimethyl-1¹H,2¹H,6¹H-10-oxa-4,8-dithia-1(7,3)-indola-2(4,3),6(3,5)-dipyrazola-9(3,1)-naphthalenacyclotridecaphane-1²-carboxylic acid

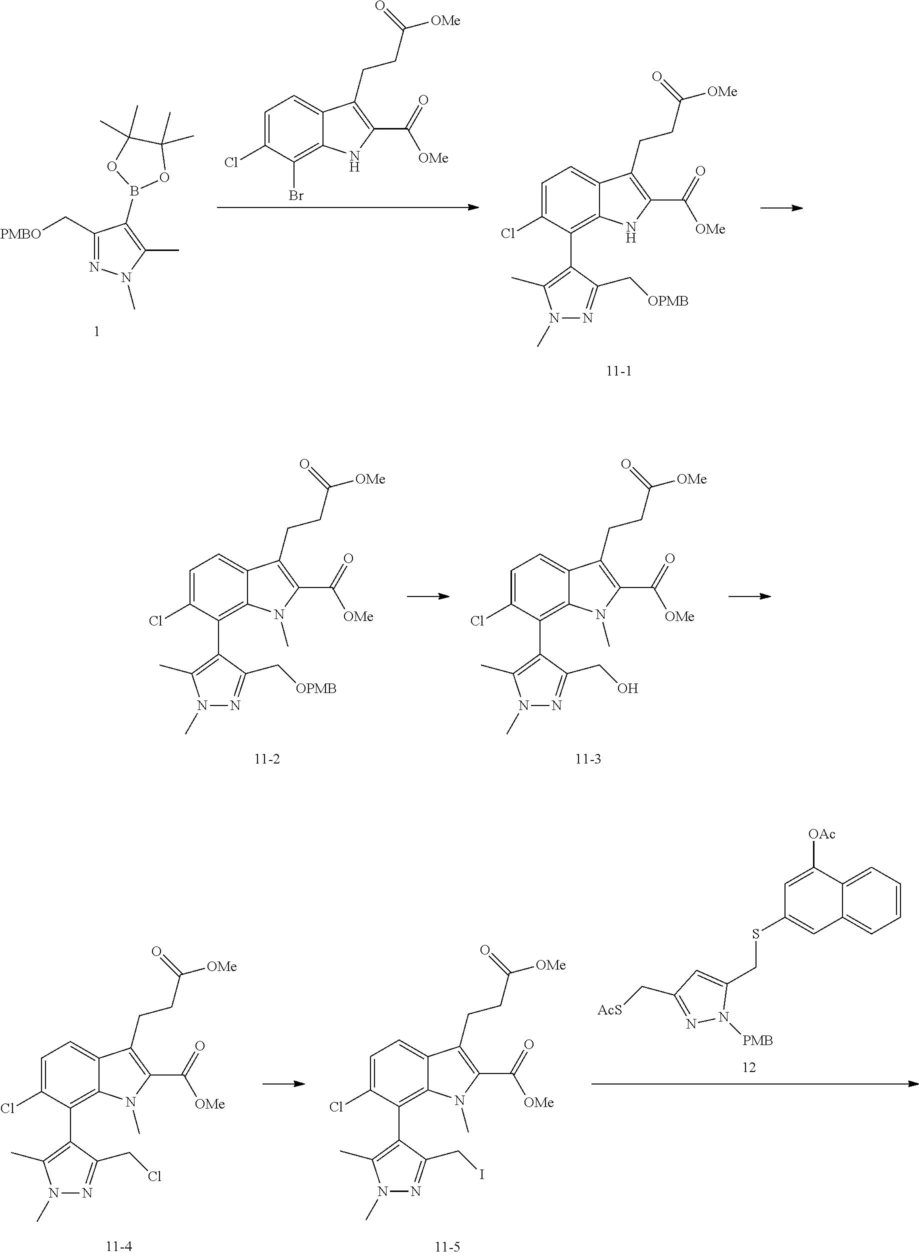

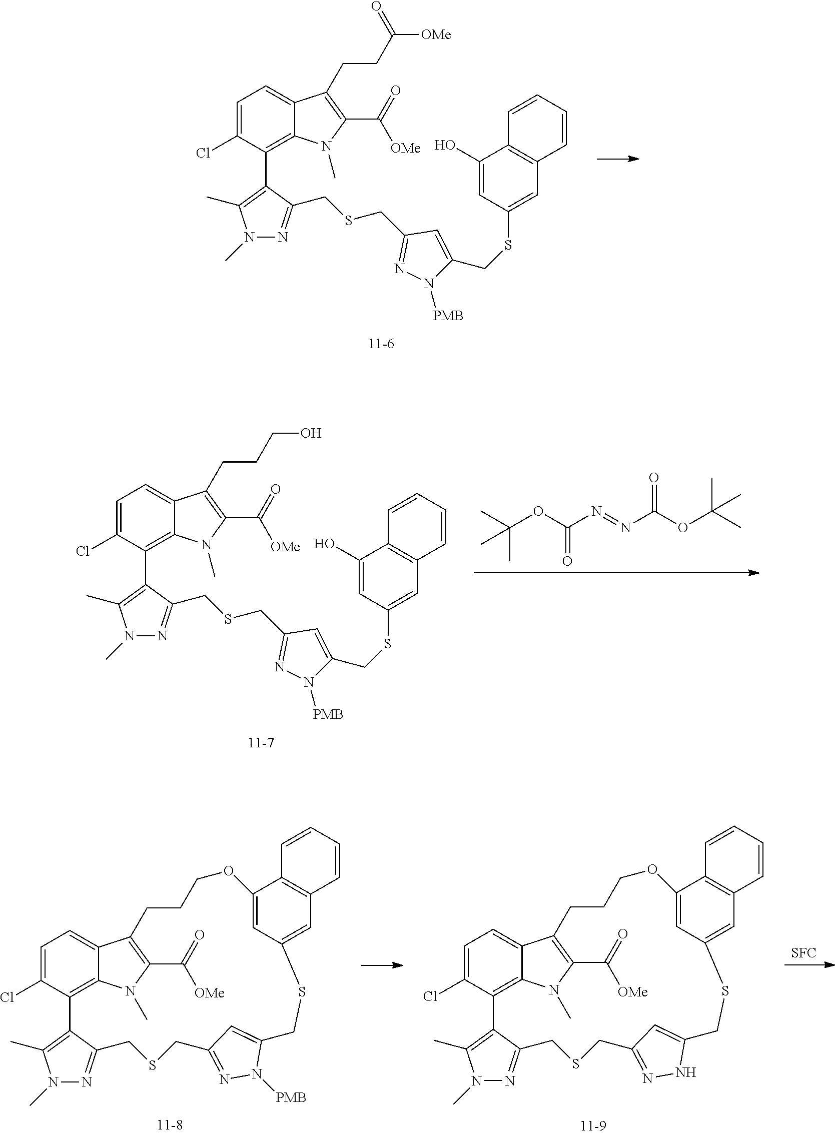

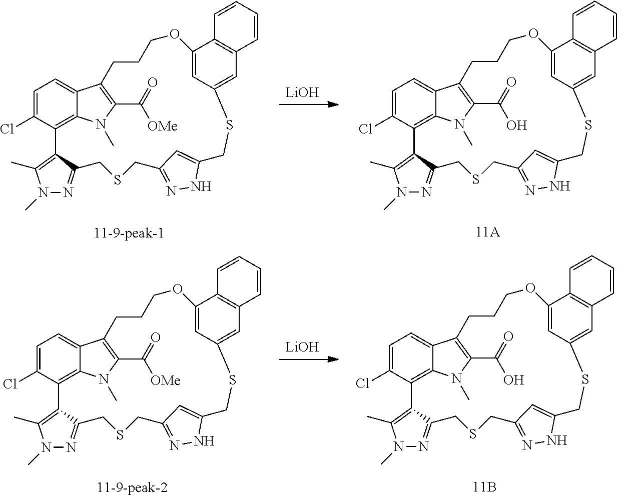

To the degassed solution of methyl 7-bromo-6-chloro-3-(3-methoxy-3-oxopropyl)-1H-indole-2-carboxylate (5 g, 13.40 mmol) in 1,4-dioxane:water (50 mL:12.5 mL) were added 3-(((4-methoxybenzyl)oxy)methyl)-1,5-dimethyl-4-(4,4,5,5-tetramethyl-1,3,2-dioxaborolan-2-yl)-¹H-pyrazole (1, 6.23 g, 16.75 mmol) and Cs₂CO₃ (8.739 g, 26.80 mmol). The solution was degassed with Ar for 15.min, followed by addition of dichloro[1,1′-bis(di-tert-butylphosphino)ferrocene] palladium(II) (0.26 g, 0.40 mmol). The solution was degassed again for 15 mins. The mixture was heated at 100° C. for 4 h. After completion of reaction, the mixture was diluted with water (100 mL) and extracted with EtOAc (3×100 mL). The combined organic layers were dried over Na₂SO₄, filtered and concentrated under reduced pressure to afford crude 11-1 that was purified by silica gel (100-200 mesh) column chromatography eluting with 70% EtOAc in PE to afford methyl 6-chloro-3-(3-methoxy-3-oxopropyl)-7-(3-(((4-methoxybenzyl)oxy)methyl)-1,5-dimethyl-1H-pyrazol-4-yl)-1H-indole-2-carboxylate (11-1, 4 g, 7.95 mmol, 55%) as a brown colored liquid. MS (LCMS) m/z 540.83 [M+H]⁺. ¹H NMR (400 MHz, CDCl₃) δ 9.17 (s, 1H), 7.63 (d, J=8.8 Hz, 1H), 7.30-7.23 (m, 2H), 7.02-6.98 (m, 2H), 6.76-6.72 (m, 2H), 4.50-4.35 (m, 4H), 4.13 (d, J=11.2 Hz, 1H), 3.90 (s, 3H), 3.80-3.70 (m, 7H), 3.67 (s, 3H), 3.45-3.40 (m, 2H), 2.72 (t, J=7.8 Hz, 2H) 2.05 (s, 3H).

To a stirred solution of methyl 6-chloro-3-(3-methoxy-3-oxopropyl)-7-(3-(((4-methoxybenzyl)oxy)methyl)-1,5-dimethyl-1H-pyrazol-4-yl)-1H-indole-2-carboxylate (11-1, 10 g, 18.51 mmol) in dry DMF (100 mL) were added Cs₂CO₃ (18.1 g, 55.55 mmol) followed by Mel (13.05 g, 92.59 mmol), and the mixture was stirred rt for 2 h. The reaction was quenched with water (200 mL) and extracted with EtOAc (3×200 mL). The combined organic layers were dried over Na₂SO₄, filtered and concentrated under reduced pressure to afford crude methyl 6-chloro-3-(3-methoxy-3-oxopropyl)-7-(3-(((4-methoxybenzyl)oxy)methyl)-1,5-dimethyl-1H-pyrazol-4-yl)-1-methyl-1H-indole-2-carboxylate (11-2, 10 g) as a brown oil that was used without further purification. MS (LCMS) m/z 554.83 [M+H]⁺. ¹H NMR (400 MHz, CDCl₃) δ 8.02 (s, 1H), 7.62 (d, J=8.0 Hz, 1H), 7.26-7.22 (m, 2H), 6.88-6.82 (m, 2H), 6.68-6.65 (m, 2H), 4.50-4.45 (m, 1H), 4.35-4.20 (m, 4H), 3.91 (s, 3H), 3.89 (s, 3H), 3.80 (s, 1H), 3.75 (s, 4H), 3.67 (s, 3H), 3.50 (s, 3H), 3.40-3.30 (m, 2H), 2.67 (t, J=7.8 Hz, 2H), 2.06 (s, 3H).

To a stirred solution of methyl 6-chloro-3-(3-methoxy-3-oxopropyl)-7-(3-(((4-methoxybenzyl)oxy)methyl)-1,5-dimethyl-1H-pyrazol-4-yl)-1-methyl-1H-indole-2-carboxylate (11-2, 10 g, 18.05 mmol) in DCM (100 mL) was added TFA (20.5 mL, 180.50 mmol) at 0° C., and the mixture was stirred at rt for 1 h. After completion of reaction, the reaction was quenched with a NaHCO₃ solution (350 mL) and extracted with DCM (2×150 mL). The combined organic layers were dried over Na₂SO₄, filtered and concentrated under reduced pressure to afford crude 11-3 that was purified by silica gel (100-200 mesh) column chromatography eluting with 8% MeOH in DCM to afford methyl 6-chloro-7-(3-(hydroxymethyl)-1,5-dimethyl-1H-pyrazol-4-yl)-3-(3-methoxy-3-oxopropyl)-1-methyl-1H-indole-2-carboxylate (11-3, 5.5 g, 12.67 mmol, 70%) as a brown semi solid. MS (LCMS) m/z 434.10 [M+H]⁺. ¹H NMR (400 MHz, CDCl₃) δ 7.63 (d, J=8.4 Hz, 1H), 7.26-7.24 (m, 1H), 4.50-4.40 (m, 2H), 3.92 (s, 3H), 3.89 (s, 3H), 3.78 (s, 1H), 3.68 (s, 3H), 3.53 (s, 3H), 3.49 (s, 3H), 3.35-3.31 (m 2H), 2.70-2.65 (m, 2H), 2.05 (s, 3H).

To a stirred solution of methyl 6-chloro-7-(3-(hydroxymethyl)-1,5-dimethyl-1H-pyrazol-4-yl)-3-(3-methoxy-3-oxopropyl)-1-methyl-1H-indole-2-carboxylate (11-3, 10 g, 23.04 mmol) in DCM (100 mL) under Ar was added SOCl₂ (2 mL, 27.65 mmol) at 0° C., and the mixture was stirred at rt for 1 h. The reaction was quenched with NaHCO₃ (100 mL) and DCM (2×200 mL). The organic layers were dried over Na₂SO₄, filtered and concentrated under reduced pressure to afford crude methyl 6-chloro-7-(3-(chloromethyl)-1,5-dimethyl-1H-pyrazol-4-yl)-3-(3-methoxy-3-oxopropyl)-1-methyl-1H-indole-2-carboxylate (11-4, 10 g) as a brown oil that was used without further purification. MS (LCMS) m/z 452.34 [M+H]⁺. ¹H NMR (400 MHz, CDCl₃) δ 7.65 (d, J=8.8 Hz, 1H), 7.26 (d, J=8.4 Hz, 1H), 4.50-4.37 (m, 2H), 3.93 (s, 3H), 3.87 (s, 3H), 3.68 (s, 3H), 3.55 (s, 3H), 3.37-3.32 (m, 2H), 2.67 (t, J=7.8 Hz, 2H), 2.05 (s, 3H).

To a stirred solution of methyl 6-chloro-7-(3-(chloromethyl)-1,5-dimethyl-1H-pyrazol-4-yl)-3-(3-methoxy-3-oxopropyl)-1-methyl-1H-indole-2-carboxylate (11-4, 10 g, 22.17 mmol) in dry MeCN (100 mL) was added NaI (5.98 g, 39.91 mmol) at rt, and the mixture was heated to 80° C. for 3 h. After completion of reaction, the mixture was diluted with water (150 mL) and extracted with EtOAc (2×200 mL). The combined organic layers were separated, dried over Na₂SO₄, filtered and concentrated under reduced pressure to afford crude of methyl 6-chloro-7-(3-(iodomethyl)-1,5-dimethyl-1H-pyrazol-4-yl)-3-(3-methoxy-3-oxopropyl)-1-methyl-1H-indole-2-carboxylate (11-5, 10 g) as a brown oil that was used without further purification. MS (LCMS) m/z 544.36 [M+H]⁺. ¹H NMR (400 MHz, CDCl₃) δ 7.66 (d, J=8.4 Hz, 1H), 7.27 (d, J=8.4 Hz, 1H), 4.25-4.09 (m, 2H), 3.93 (s, 3H), 3.87 (s, 3H), 3.67 (s, 3H), 3.58 (s, 3H), 3.38-3.33 (m, 2H), 2.70-2.66 (m, 2H), 2.05 (s, 3H).

To a stirred solution of methyl 6-chloro-7-(3-(iodomethyl)-1,5-dimethyl-1H-pyrazol-4-yl)-3-(3-methoxy-3-oxopropyl)-1-methyl-1H-indole-2-carboxylate (11-5, 5 g, 9.19 mmol) in MeOH (50 mL) was added K₂CO₃ (5.1 g, 36.76 mmol), and the solution was degassed with Ar for 15 min. In another round bottom flask, 3-(((3-((acetylthio)methyl)-1-(4-methoxybenzyl)-1H-pyrazol-5-yl)methyl)thio)naphthalen-1-yl acetate (12, 4.6 g, 9.19 mmol) in methanol (20 mL) was degassed with Ar for 15 min, and this solution was added to above mixture dropwise. The mixture was stirred at rt for 16 h. The mixture was diluted with water (150 mL) and extracted with EtOAc (2×200 mL). The combined organic layers were separated, dried over Na₂SO₄, filtered and concentrated under reduced pressure to afford crude 11-6 that was purified by silica gel (100-200 mesh) column chromatography eluting with 60% EtOAc in PE to afford methyl 6-chloro-7-(3-((((5-(((4-hydroxynaphthalen-2-yl)thio)methyl)-1-(4-methoxybenzyl)-1H-pyrazol-3-yl)methyl)thio)methyl)-1,5-dimethyl-1H-pyrazol-4-yl)-3-(3-methoxy-3-oxopropyl)-1-methyl-1H-indole-2-carboxylate (11-6, 4.3 g, 5.72 mmol, 62%) as a light brown oil. MS (LCMS) m/z 838.53 [M+H]⁺. ¹H NMR (400 MHz, CDCl₃) δ 8.29-8.26 (m, 1H), 7.76-7.65 (m, 1H), 7.63-7.60 (m, 2H), 7.50-7.47 (m, 2H), 7.27-7.25 (m, 2H), 6.92 (d, J=8.8 Hz, 2H), 6.74-6.70 (m, 2H), 6.67 (s, 1H), 6.02 (s, 1H), 5.01 (s, 1H), 3.93 (s, 3H), 3.89 (, 3H), 3.80 (s, 2H), 3.72 (s, 3H), 3.70 (s, 3H), 3.60 (s, 3H), 3.40-3.30 (m, 2H), 2.71 (t, J=7.8 Hz, 2H), 2.05 (s, 3H).

To a stirred solution of methyl 6-chloro-7-(3-((((5-(((4-hydroxynaphthalen-2-yl)thio)methyl)-1-(4-methoxybenzyl)-1H-pyrazol-3-yl)methyl)thio)methyl)-1,5-dimethyl-1H-pyrazol-4-yl)-3-(3-methoxy-3-oxopropyl)-1-methyl-1H-indole-2-carboxylate (11-6, 2.5 g, 2.98 mmol) in THF (25.0 mL) was added BH₃.THF (1M in THF) (15 mL, 14.92 mmol) at 0° C., and the mixture was stirred at 70° C. for 16 h. After completion of starting material, the reaction was quenched with MeOH (20 mL) and reflux for 1 h. The mixture was concentrated, diluted with water (50 mL) and extracted with EtOAc (2×150 mL). The combined organic layers were dried over Na₂SO₄, filtered, concentrated under reduced pressure to afford crude 11-7 that was purified by silica gel (100-200 mesh) column chromatography eluting with 70% EtOAc in PE to afford methyl 6-chloro-7-(3-((((5-(((4-hydroxynaphthalen-2-yl)thio)methyl)-1-(4-methoxybenzyl)-1H-pyrazol-3-yl)methyl)thio)methyl)-1,5-dimethyl-1H-pyrazol-4-yl)-3-(3-hydroxypropyl)-1-methyl-1H-indole-2-carboxylate (11-7, 1.3 g, 1.60 mmol, 53%) as a brown solid. MS (LCMS) m/z 810.67 [M+H]⁺. ¹H NMR (400 MHz, CDCl₃) δ 9.80 (br s, 1H), 8.30-8.25 (m, 1H), 7.76-7.73 (m, 1H), 7.65-7.60 (m, 2H), 7.52-7.47 (m, 2H), 7.26-7.24 (m, 1H), 6.92 (d, J=8.8 Hz, 2H), 6.74-6.70 (m, 2H), 6.66 (s, 1H), 6.02 (s, 1H), 5.01 (s, 2H), 3.93 (s, 3H), 3.90 (s, 3H), 3.81 (s, 2H), 3.68 (s, 3H), 3.66-3.63 (m, 2H), 3.60-3.48 (m, 8H), 3.16 (t, J=7.2 Hz, 2H), 2.10-2.04 (m, 4H), 1.99-1.94 (m, 2H).

To a stirred solution of TPP (0.38 g, 0.74 mmol) in toluene (2 mL) was added a solution of di-tert-butyl (E)-diazene-1,2-dicarboxylate (0.34 g, 1.48 mmol) and methyl 6-chloro-7-(3-((((5-(((4-hydroxynaphthalen-2-yl)thio)methyl)-1-(4-methoxybenzyl)-1H-pyrazol-3-yl)methyl)thio)methyl)-1,5-dimethyl-1H-pyrazol-4-yl)-3-(3-hydroxypropyl)-1-methyl-1H-indole-2-carboxylate (0.6 g, 0.74 mmol) in toluene (8 mL) and THF (2 mL) at 70° C. The mixture was stirred at 70° C. for 16 h. After completion of reaction, the reaction was quenched with water (20 mL) and extracted with EtOAc (2×50 mL). The combined organic layers were dried over Na₂SO₄, filtered and concentrated under reduced pressure to afford crude 11-8 that was purified by flash column chromatography eluting with 60% EtOAc in PE to afford methyl (Z)-1⁶-chloro-6¹-(4-methoxybenzyl)-1¹,2¹,2⁵-trimethyl-1¹H,2¹H,6¹H-10-oxa-4,8-dithia-1(7,3)-indola-2(4,3),6(3,5)-dipyrazola-9(3,1)-naphthalenacyclotridecaphane-1²-carboxylate (11-8, 0.25 g, 0.316 mmol, 43%) as an off white solid. MS (LCMS) m/z 792.46 [M+H]⁺.

To a stirred solution of methyl (Z)-1⁶-chloro-6¹-(4-methoxybenzyl)-1¹,2¹,2⁵-trimethyl-1¹H,2¹H,6¹H-10-oxa-4,8-dithia-1(7,3)-indola-2(4,3),6(3,5)-dipyrazola-9(3,1)-naphthalenacyclotridecaphane-1²-carboxylate (11-8, 0.8 g, 1.01 mmol) in TFA (12 mL) was added anisole (0.65 mL, 6.06 mmol) at rt, and the mixture was stirred at 80° C. for 16 h. After completion of reaction, the mixture was concentrated under reduced pressure to afford crude 11-9 that was purified by flash column chromatography (reverse phase) eluting with 40% ACN in water to afford still crude 11-9 that was purified by Prep-HPLC. The collected fractions (200 mL) were concentrated under reduced pressure to afford methyl (Z)-1⁶-chloro-1¹,2¹,2⁵-trimethyl-1¹H,2¹H,6¹H-10-oxa-4,8-dithia-1(7,3)-indola-2(4,3),6(3,5)-dipyrazola-9(3,1)-naphthalenacyclotridecaphane-1²-carboxylate (11-9, 0.36 g, 0.536 mmol, 33%) as an off white solid. MS (LCMS) m/z 672.37 [M+H]⁺. ¹H NMR (400 MHz, CDCl₃) δ 8.32-8.29 (m, 1H), 7.73-7.70 (m, 2H), 7.53-7.47 (m, 4H), 6.98 (d, J=8.4 Hz, 1H), 6.46 (s, 1H), 5.39 (s, 1H), 4.05-3.95 (m, 2H), 3.90-3.75 (m, 7H), 3.60 (s, 3H), 3.50-3.45 (m, 4H), 3.35-3.20 (m, 3H), 3.03 (d, J=14.8 Hz, 1H), 2.48-2.40 (m, 1H), 2.30-2.20 (m, 1H), 2.05 (s, 3H).

Methyl (Z)-1⁶-chloro-1¹,2¹,2⁵-trimethyl-1¹H,2¹H,6¹H-10-oxa-4,8-dithia-1(7,3)-indola-2(4,3),6(3,5)-dipyrazola-9(3,1)-naphthalenacyclotridecaphane-1²-carboxylate (11-9, 360 mg) was purified by SFC purification to afford 11-9-peak-1 (150 mg) as an off white solid with 99.02% of LCMS purity (chiral HPLC: 99.86%) and 11-9-Peak-2 (150 mg) as an off white solid with 96.54% of LCMS purity (chiral HPLC:99.04%). These two peaks were separately used for the next steps to get respective final compounds.

11-9-peak-1: MS (LCMS) m/z 672.30 [M+H]⁺. ¹H NMR (400 MHz, CDCl₃) δ 8.32-8.29 (m, 1H), 7.73-7.70 (m, 2H), 7.53-7.46 (m, 4H), 6.98 (d, J=8.8 Hz, 1H), 6.46 (s, 1H), 5.39 (s, 1H), 4.05-3.95 (m, 2H), 3.90-3.75 (m, 7H), 3.60 (s, 3H), 3.50-3.45 (m, 4H), 3.35-3.20 (m, 3H), 3.03 (d, J=14.4 Hz, 1H), 2.48-2.40 (m, 1H), 2.30-2.20 (m, 1H), 2.05 (s, 3H).

11-9-peak-2: MS (LCMS) m/z 672.26 [M+H]⁺. ¹H NMR (400 MHz, CDCl₃) δ 8.32-8.29 (m, 1H), 7.73-7.70 (m, 2H), 7.53-7.47 (m, 4H), 6.98 (d, J=8.8 Hz, 1H), 6.46 (s, 1H), 5.41 (s, 1H), 4.05-3.95 (m, 2H), 3.90-3.75 (m, 7H), 3.61 (s, 3H), 3.50-3.45 (m, 4H), 3.35-3.20 (m, 3H), 3.03 (d, J=14.4 Hz, 1H), 2.48-2.40 (m, 1H), 2.30-2.20 (m, 1H), 2.05 (s, 3H).

To a stirred solution of methyl (Z)-1⁶-chloro-1¹,2¹,2⁵-trimethyl-1¹H,2¹H,6¹H-10-oxa-4,8-dithia-1(7,3)-indola-2(4,3),6(3,5)-dipyrazola-9(3,1)-naphthalenacyclotridecaphane-1²-carboxylate (11-9-peak-1, 2×75 mg, 0.116 mmol) in THF:H₂O (1:1, 3 mL) was added LiOH.H₂O in 3 equal portions in 3 h intervals (70.3 mg, 1.674 mmol) at 0° C. The mixture was stirred at rt for 16 h. After completion of reaction, the mixture was acidified to pH 2 using 2 N aqueous HCl (5 mL) and extracted with EtOAc (2×10 mL). The combined organic layers were dried under reduced pressure to afford crude 11A that was purified by Prep-TLC eluting with 7% MeOH in DCM to afford 11A. Compound 11A was dissolved in ACN (1 mL), water (1 mL), frozen and lyophilized to afford (Z)-1⁶-chloro-1¹,2¹,2⁵-trimethyl-1¹H,2¹H,6¹H-10-oxa-4,8-dithia-1(7,3)-indola-2(4,3),6(3,5)-dipyrazola-9(3,1)-naphthalenacyclotridecaphane-1²-carboxylic acid (11A, 70.3 mg, 0.106 mmol, 50%) as a white solid. MS (LCMS) m/z 658.29 [M+H]⁺. ¹H NMR (400 MHz, DMSO-d₆) δ 12.7 (s, 1H), 8.10-8.05 (m, 1H), 7.75-7.65 (m, 1H), 7.64-7.50 (m, 1H), 7.50-7.30 (m, 3H), 7.00 (br s, 1H), 6.80-6.50 (br s, 1H), 5.10 (br s, 1H), 4.40-3.90 (m, 7H), 3.60-3.40 (m, 5H), 3.40-2.80 (m, 5H), 2.30-2.20 (m, 2H), 1.98 (s, 3H); LCMS purity: 98.12%. HPLC purity: 99.11%; Chiral HPLC purity: 99.82%.

To a stirred solution of methyl (Z)-1⁶-chloro-1¹,2¹,2⁵-trimethyl-1¹H,2¹H,6¹H-10-oxa-4,8-dithia-1(7,3)-indola-2(4,3),6(3,5)-dipyrazola-9(3,1)-naphthalenacyclotridecaphane-1²-carboxylate (11-9-peak-2, 2×75 mg, 0.223 mmol) in THF:H₂O (1:1, 3 mL) was added LiOH.H₂O in 3 equal portions in 3 h intervals (70.3 mg, 1.674 mmol) at 0° C. The mixture was stirred at rt for 16 h. After completion of reaction, the mixture was acidified to pH 2 using 2 N aqueous HCl (5 mL) and extracted with EtOAc (2×10 mL). The combined organic layers were dried under reduced pressure to afford crude 11B that was purified by Prep-TLC eluting with 7% MeOH in DCM to afford 11B. Compound 11B was dissolved in ACN (1 mL), water (1 mL) frozen and lyophilized to afford (Z)-1⁶-chloro-1¹,2¹,2⁵-trimethyl-1¹H,2¹H,6¹H-10-oxa-4,8-dithia-1(7,3)-indola-2(4,3),6(3,5)-dipyrazola-9(3,1)-naphthalenacyclotridecaphane-1²-carboxylic acid (11B, 81.8 mg, 0.123 mmol, 58%) as a white solid. MS (LCMS) m/z 658.29 [M+H]⁺. ¹H NMR (400 MHz, DMSO-d₆) δ 12.5 (br s, 1H), 8.10-8.05 (m, 1H), 7.73-7.60 (m, 2H), 7.50-7.40 (m, 2H), 7.35 (s, 1H), 7.02 (d, J=8.4 Hz, 1H), 6.66 (br s, 1H), 4.99 (s, 1H), 4.25-3.85 (m, 4H), 3.75 (s, 3H), 3.60-3.40 (m, 4H), 3.40-3.10 (m, 2H), 3.05-2.90 (m, 2H), 2.35-2.20 (m, 2H), 1.98 (s, 3H); LCMS purity: 98.07%. HPLC purity: 99.40%; Chiral HPLC purity: 98.63%.

The absolute stereochemistry of compounds (11A) and (11B) is arbitrarily assigned.

### Example 12

### (Z)-1⁶,9⁷-Dichloro-1¹,2¹,2⁵,6¹-tetramethyl-1¹H,2¹H,6¹H-10-oxa-4,8-dithia-1(7,3)-indola-2(4,3),6(3,5)-dipyrazola-9(3,1)-naphthalenacyclotridecaphane-1²-carboxylic acid

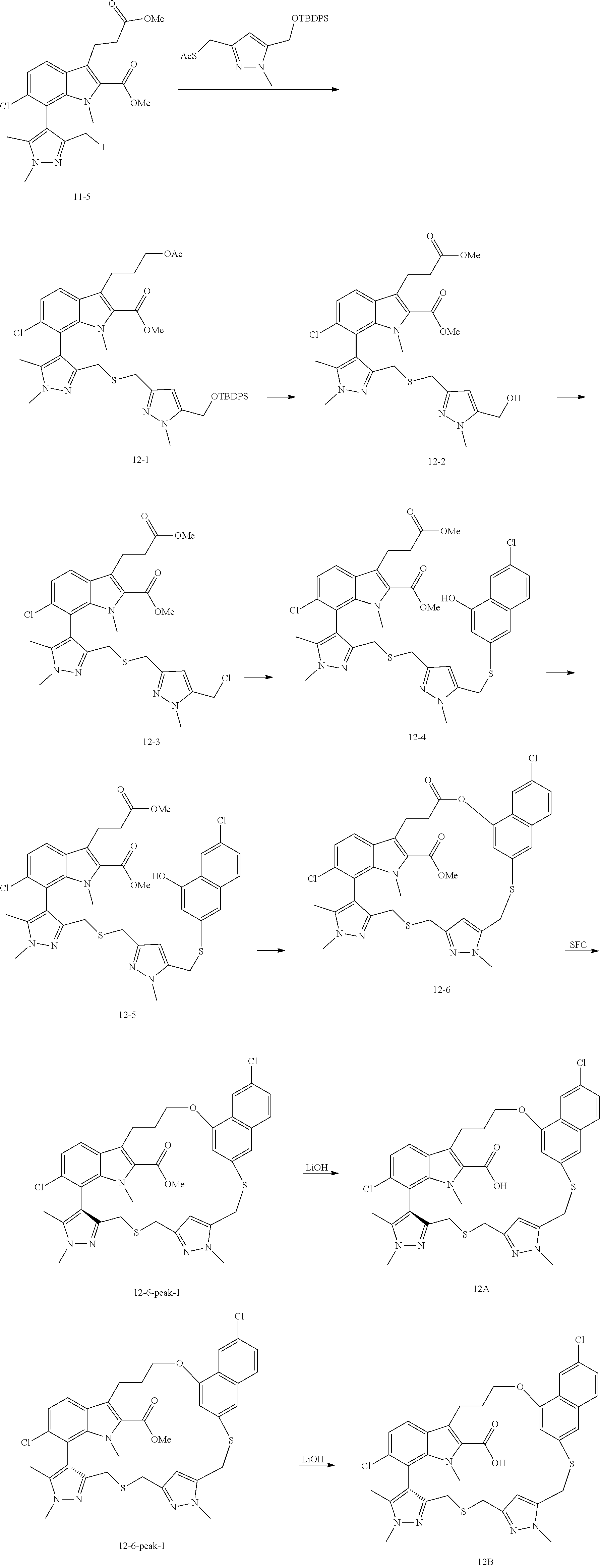

To a stirred solution of semi pure methyl 6-chloro-7-(3-(iodomethyl)-1,5-dimethyl-1H-pyrazol-4-yl)-3-(3-methoxy-3-oxopropyl)-1-methyl-1H-indole-2-carboxylate from example 11 (11-5, 8 g, 14.718 mmol) in MeOH (40 mL), THF (15 mL) and K₂CO₃ (2.03 g, 14.718 mmol) were added. The solution was degassed with Ar for 10 min. In another round bottom flask, S-((5-(((tert-butyldiphenylsilyl)oxy)methyl)-1-methyl-1H-pyrazol-3-yl)methyl) ethanethioate (3, 7.09 g, 16.183 mmol) in methanol (40 mL) was degassed with Ar for 10 min, and this solution was added to previous mixture dropwise. The mixture was stirred at rt for 16 h. The solvent was evaporated, and the mixture was diluted with water (250 mL) and extracted with EtOAc (3×500 mL). The organic layer was separated, dried over Na₂SO₄, filtered and evaporated to get semi pure 12-1 that was purified by silica gel column chromatography using 50-70% EtOAc PE to afford methyl 7-(3-((((5-(((tert-butyldiphenylsilyl)oxy)methyl)-1-methyl-1H-pyrazol-3-yl)methyl)thio)methyl)-1,5-dimethyl-1H-pyrazol-4-yl)-6-chloro-3-(3-methoxy-3-oxopropyl)-1-methyl-1H-indole-2-carboxylate (12-1, 6.5 g, 8.014 mmol, 48% for three steps) as a light brown liquid. MS (LCMS) m/z 812.19 [M+H]⁺. ¹H NMR (400 MHz, CDCl₃) δ 7.68-7.62 (m, 4H), 7.56 (d, J=8.8 Hz, 1H), 7.48-7.35 (m, 6H), 7.20 (d, J=8.4 Hz, 1H), 5.86 (s, 1H), 4.55 (s, 2H), 3.90 (s, 3H), 3.86 (s, 3H), 3.73 (s, 3H), 3.68 (s, 3H), 3.61-3.48 (m, 6H), 3.40-3.25 (m, 2H), 2.64 (t, J=8.0 Hz, 2H), 2.02 (s, 3H), 1.04 (s, 9H).

To a stirred solution of methyl 3-(3-acetoxypropyl)-7-(2-((((5-(((tert-butyldiphenylsilyl)oxy)methyl)-1-methyl-1H-pyrazol-3-yl)methyl)thio)methyl)-4,5,6,7-tetrahydropyrazolo[1,5-a]pyridin-3-yl)-6-chloro-1-methyl-1H-indole-2-carboxylate (12-1, (6.5 g, 8.00 mmol) in THF (90 mL) was added TBAF (1 M in THF, 8.0 mL, 8.00 mmol) at 0° C., and the mixture was stirred at rt for 2 h. The reaction was quenched with water (200 mL) and extracted with EtOAc (3×500 mL). The organic layer was washed with water (200 mL) and brine (250 mL). The organic layer was separated, dried over Na₂SO₄, filtered and evaporated to get semi pure 12-2 that was purified by silica gel column chromatography using 5% MeOH in DCM to afford methyl 6-chloro-7-(3-((((5-(hydroxymethyl)-1-methyl-1H-pyrazol-3-yl)methyl)thio)methyl)-1,5-dimethyl-1H-pyrazol-4-yl)-3-(3-methoxy-3-xopropyl)-1-methyl-1H-indole-2-carboxylate (12-2, 3.1 g, 5.399 mmol, 67%) as a brown semi solid. MS (LCMS) m/z 574.57 [M+H]⁺. ¹H NMR (400 MHz, CDCl₃) δ 7.62 (d, J=8.8 Hz, 1H), 7.24 (d, J=8.4 Hz, 1H), 5.92 (s, 1H), 4.54 (s, 2H), 3.92 (s, 3H), 3.85 (s, 3H), 3.76 (s, 3H), 3.64 (s, 3H), 3.60-3.45 (m, 7H), 3.40-3.30 (m, 2H), 2.67 (t, J=8.0 Hz, 2H), 2.04 (s, 3H).

To a stirred solution of methyl 6-chloro-7-(3-((((5-(hydroxymethyl)-1-methyl-1H-pyrazol-3-yl)methyl)thio)methyl)-1,5-dimethyl-1H-pyrazol-4-yl)-3-(3-methoxy-3-oxopropyl)-1-methyl-1H-indole-2-carboxylate (12-2, 3.1 g, 5.399 mmol) in DCM (50 mL) under Ar was added SOCl₂ (764 mg, 6.478 mmol) at 0° C., and the mixture was stirred at rt for 1 h. The mixture was diluted with DCM (300 mL) and washed with a sat. NaHCO₃ solution (3×100 mL). The organic layer was separated, dried over Na₂SO₄, filtered and evaporated to afford semi pure methyl 6-chloro-7-(3-((((5-(chloromethyl)-1-methyl-1H-pyrazol-3-yl)methyl)thio)methyl)-1,5-dimethyl-1H-pyrazol-4-yl)-3-(3-methoxy-3-oxopropyl)-1-methyl-1H-indole-2-carboxylate (12-3, 3.1 g, 5.231 mmol) as a brown semi solid that was used without further purification. MS (LCMS) m/z 594.44 [M+H]⁺.

To a stirred solution of semi pure methyl 6-chloro-7-(3-((((5-(chloromethyl)-1-methyl-1H-pyrazol-3-yl)methyl)thio)methyl)-1,5-dimethyl-1H-pyrazol-4-yl)-3-(3-methoxy-3-oxopropyl)-1-methyl-1H-indole-2-carboxylate (12-3, 3 g, 5.063 mmol) in MeOH (20 mL) was added K₂CO₃ (1.64 g, 12.150 mmol), and the solution was degassed with Ar for 10 min. In another round bottom flask, 3-(acetylthio)-7-chloronaphthalen-1-yl acetate (made from the same procedure as intermediate 4) (1.64 g, 5.5692 mmol) in methanol (15 mL) was degassed with Ar for 10 min, and this solution was added to previous mixture dropwise. The mixture was stirred at rt for 1 h. The solvent was evaporated, and the mixture was diluted with water (200 mL) and extracted with EtOAc (2×500 mL). The organic layer was separated, dried over Na₂SO₄, filtered and evaporated to get semi pure 12-4 that was purified by silica gel column chromatography using 3% MeOH in DCM to afford methyl 6-chloro-7-(3-((((5-(((6-chloro-4-hydroxynaphthalen-2-yl)thio)methyl)-1-methyl-1H-pyrazol-3-yl)methyl)thio)methyl)-1,5-dimethyl-1H-pyrazol-4-yl)-3-(3-methoxy-3-oxopropyl)-1-methyl-1H-indole-2-carboxylate (12-4, 2.5 g, 3.26 mmol, 60% for two steps) as a light brown solid. MS (LCMS) m/z 766.08 [M+H]⁺. ¹H NMR (400 MHz, CDCl₃) δ 10.18 (s, 1H), 8.26 (d, J=2.0 Hz, 1H), 7.70-7.63 (m, 3H), 7.44-7.42 (dd, J=2.2 Hz and 8.6 Hz, 1H), 7.27 (d, J=9.6 Hz, 1H, merged with solvent peak), 6.60 (d, J=1.6 Hz, 1H), 5.95 (s, 1H), 3.95-3.92 (m, 9H), 3.76 (s, 3H), 3.60-3.55 (m, 4H), 3.52-3.49 (m, 5H), 4.45 (d, J=8.0 Hz, 2H), 3.40-3.35 (m, 3H), 2.07 (s, 3H).

To a stirred solution of methyl 6-chloro-7-(3-((((5-(((6-chloro-4-hydroxynaphthalen-2-yl)thio)methyl)-1-methyl-1H-pyrazol-3-yl)methyl)thio)methyl)-1,5-dimethyl-1H-pyrazol-4-yl)-3-(3-methoxy-3-oxopropyl)-1-methyl-1H-indole-2-carboxylate (12-4, 2.5 g, 3.2604 mmol) in THF (40 mL) was added BH₃.THF (1M in THF) (17.9 mL, 17.93 mmol) at 0° C., and the mixture was stirred at rt for 6 h. The volatiles were removed, and the reaction was quenched with MeOH (5 mL) and 6N HCl (10 mL). The mixture was stirred for 30 min and extracted with EtOAc (2×500 mL). The organic layer was dried over Na₂SO₄, filtered, concentrated to give semi pure 12-5 that was purified by silica gel column chromatography using 6% MeOH in DCM to afford methyl 6-chloro-7-(3-((((5-(((6-chloro-4-hydroxynaphthalen-2-yl)thio)methyl)-1-methyl-1H-pyrazol-3-yl)methyl)thio)methyl)-1,5-dimethyl-1H-pyrazol-4-yl)-3-(3-hydroxypropyl)-1-methyl-1H-indole-2-carboxylate (12-5, 1.6 g, 2.165 mmol, 66%) as an brown sticky solid. MS (LCMS) m/z 738.12 [M+H]⁺. ¹H NMR (400 MHz, CDCl₃) δ 10.20 (brs, 1H), 8.26 (d, J=2.4 Hz, 1H), 7.70-7.62 (m, 3H), 7.44-7.42 (dd, J=2.4 Hz and 8.8 Hz, 1H), 7.30-7.25 (m, 1H, merged with solvent peak), 6.61 (s, 1H), 5.95 (s, 1H), 3.95-3.90 (m, 9H), 3.87 (brs, 2H), 3.60-3.40 (m, 11H), 3.20-3.15 (m, 2H), 2.31 (brs, 1H), 2.08 (s, 3H), 1.99-1.95 (m, 1H).

To a stirred solution of TPP (355 mg, 1.3543 mmol) in toluene (10 mL) was added a solution of di-tert-butyl diazene-1,2-dicarboxylate (311 mg, 1.3543 mmol) and methyl 6-chloro-7-(3-((((5-(((6-chloro-4-hydroxynaphthalen-2-yl)thio)methyl)-1-methyl-1H-pyrazol-3-yl)methyl) thio)methyl)-1,5-dimethyl-1H-pyrazol-4-yl)-3-(3-hydroxypropyl)-1-methyl-1H-indole-2-carboxylate (12-5, 2×500 mg, 0.676 mmol) in toluene (5 mL) and THF (2 mL), and the mixture was stirred at rt for 2 h. The reaction was quenched with water (50 mL) and extracted with EtOAc (2×250 mL). The organic layer was dried over Na₂SO₄, filtered, concentrated to give semi pure 12-6 that was purified by prep-HPLC to afford methyl (Z)-1⁶,9⁷-dichloro-1¹,2¹,2⁵,6¹-tetramethyl-1¹H,2¹H,6¹H-10-oxa-4,8-dithia-1(7,3)-indola-2(4,3),6(3,5)-dipyrazola-9(3,1)-naphthalenacyclotridecaphane-12-carboxylate (12-6, 450 mg, 0.624 mmol, 46%) as an off white sticky solid. MS (LCMS) m/z 720.45 [M+H]⁺. ¹H NMR (400 MHz, CDCl₃) δ 8.24 (s, 1H), 7.65 (d, J=8.8 Hz, 1H), 7.53 (d, J=6.8 Hz, 1H), 7.46-7.42 (m, 2H), 7.00 (d, J=8.4 Hz, 1H), 6.29 (s, 1H), 4.88 (s, 1H), 3.90-3.86 (m, 4H), 3.84 (s, 3H), 3.74 (s, 3H), 3.65 (s, 3H), 3.05-2.96 (m, 3H), 3.43 (d, J=14.0 Hz, 1H), 3.25-3.18 (m, 2H), 3.08 (d, J=13.6 Hz, 1H), 2.70 (d, J=14.0 Hz, 1H), 2.50-2.40 (m, 1H), 2.30-2.15 (m, 1H), 2.04 (s, 3H).

Methyl (Z)-1⁶,9⁷-dichloro-1¹,2¹,2⁵,6¹-tetramethyl-1¹H,2¹H,6¹H-10-oxa-4,8-dithia-1(7,3)-indola-2(4,3),6(3,5)-dipyrazola-9(3,1)-naphthalenacyclotridecaphane-12-carboxylate (12-6, 450 mg) was purified by SFC purification to afford 12-6-peak-1 (180 mg) as an off white solid with 99.22% of LCMS purity (chiral HPLC:98.73%) and 12-6-peak-2 (185 mg) as an off white solid with 99.65% of LCMS purity (chiral HPLC:99.28%). These two peaks were separately used for the next steps to get respective final compound.

12-6-peak-1: MS (LCMS) m/z 720.12 [M+H]⁺. ¹H NMR (400 MHz, CDCl₃) δ 8.24 (s, 1H), 7.65 (d, J=8.8 Hz, 1H), 7.53 (d, J=6.8 Hz, 1H), 7.46-7.42 (m, 2H), 7.00 (d, J=8.8 Hz, 1H), 6.29 (s, 1H), 4.88 (s, 1H), 3.90-3.86 (m, 4H), 3.84 (s, 3H), 3.74 (s, 3H), 3.65 (s, 3H), 3.05-2.96 (m, 3H), 3.43 (d, J=14.0 Hz, 1H), 3.25-3.18 (m, 2H), 3.08 (d, J=13.6 Hz, 1H), 2.70 (d, J=14.0 Hz, 1H), 2.50-2.40 (m, 1H), 2.30-2.15 (m, 1H), 2.04 (s, 3H).

12-6-peak-2: MS (LCMS) m/z 720.48 [M+H]⁺. ¹H NMR (400 MHz, CDCl₃) δ 8.24 (s, 1H), 7.65 (d, J=8.8 Hz, 1H), 7.53 (d, J=6.8 Hz, 1H), 7.46-7.42 (m, 2H), 7.00 (d, J=8.8 Hz, 1H), 6.29 (s, 1H), 4.88 (s, 1H), 3.90-3.86 (m, 4H), 3.84 (s, 3H), 3.74 (s, 3H), 3.65 (s, 3H), 3.05-2.96 (m, 3H), 3.43 (d, J=14.0 Hz, 1H), 3.25-3.18 (m, 2H), 3.08 (d, J=13.6 Hz, 1H), 2.70 (d, J=14.0 Hz, 1H), 2.50-2.40 (m, 1H), 2.30-2.15 (m, 1H), 2.04 (s, 3H).

To a stirred solution of methyl (Z)-1⁶,9⁷-dichloro-1¹,2¹,2⁵,6¹-tetramethyl-1¹H,2¹H,6¹H-10-oxa-4,8-dithia-1(7,3)-indola-2(4,3),6(3,5)-dipyrazola-9(3,1)-naphthalenacyclotridecaphane-12-carboxylate (12-6-peak-1, 180 mg, 0.2497 mmol) in THF:H₂O (3:1, 5 mL) was added of LiOH.H₂O in 3 equal portions in 3 h intervals (157 mg, 3.7462 mmol) at 0° C., and the mixture was stirred at rt for 16 h. The solvent was evaporated, and the aqueous layer was acidified to pH 2 using 2 N aq. HCl. The precipitate was filtered off and washed with water (2×5 mL). The solid was collected and dried under vacuum to afford ((Z)-1⁶,9⁷-dichloro-1¹,2¹,2⁵,6¹-tetramethyl-1¹H,2¹H,6¹H-10-oxa-4,8-dithia-1(7,3)-indola-2(4,3),6(3,5)-dipyrazola-9(3,1)-naphthalenacyclotridecaphane-12-carboxylic acid (12A, 130 mg, 0.183 mmol, 73%) as an off white solid. MS (LCMS) m/z 706.18 [M+H]⁺. HPLC: 95.37%. Chiral HPLC: 98.97%. ¹H NMR (400 MHz, DMSO-d₆) δ 13.3 (brs, 1H), 8.03 (d, J=2.0 Hz, 1H), 7.89 (d, J=8.4 Hz, 1H), 7.78 (d, J=8.8 Hz, 1H), 7.52-7.78 (dd, J=2.0 Hz and 8.8 Hz, 1H), 7.41 (s, 1H), 7.17 (d, J=8.8 Hz, 1H), 6.74 (s, 1H), 4.73 (s, 1H), 4.28 (s, 2H), 4.20-4.10 (m, 1H), 3.90-3.84 (m, 1H), 3.76 (s, 3H), 3.71 (s, 3H), 3.60-3.40 (m, 5H, merged with solvent residual peak), 3.16-3.07 (m, 3H), 2.91 (d, J=14.0 Hz, 1H), 2.40-2.32 (m, 1H), 2.30-2.20 (m, 1H), 1.97 (s, 3H).

To a stirred solution of methyl (Z)-1⁶,9⁷-dichloro-1¹,2¹,2⁵,6¹-tetramethyl-1H,2¹H,6¹H-10-oxa-4,8-dithia-1(7,3)-indola-2(4,3),6(3,5)-dipyrazola-9(3,1)-naphthalenacyclotridecaphane-12-carboxylate (12-6-peak-2, 180 mg, 0.2497 mmol) in THF:H₂O (3:1, 5 mL) was added of LiOH.H₂O in 3 equal portions in 3 h intervals (157 mg, 3.7462 mmol) at 0° C., and the mixture was stirred at rt for 16 h. The solvent was evaporated, and the aqueous layer was acidified to pH 2 using 2 N aq. HCl. The precipitate was filtered off and washed with water (2×5 mL). The solid was collected and dried under vacuum to afford ((Z)-1⁶,9⁷-dichloro-1¹,2¹,2⁵,6¹-tetramethyl-1¹H,2¹H,6¹H-10-oxa-4,8-dithia-1(7,3)-indola-2(4,3),6(3,5)-dipyrazola-9(3,1)-naphthalenacyclotridecaphane-12-carboxylic acid (12B, 135 mg, 0.19 mmol, 76%) as an off white solid. MS (LCMS) m/z 706.21 [M+H]⁺. HPLC: 96.10%. Chiral HPLC: 98.71%. ¹H NMR (400 MHz, DMSO-d₆) δ 13.3 (brs, 1H), 8.02 (d, J=2.0 Hz, 1H), 7.87 (d, J=8.8 Hz, 1H), 7.77 (d, J=8.8 Hz, 1H), 7.52-7.78 (dd, J=2.0 Hz and 8.8 Hz, 1H), 7.41 (s, 1H), 7.17 (d, J=8.8 Hz, 1H), 6.74 (s, 1H), 4.73 (s, 1H), 4.28 (s, 2H), 4.20-4.10 (m, 1H), 3.90-3.84 (m, 1H), 3.76 (s, 3H), 3.71 (s, 3H), 3.49 (s, 3H), 3.60-3.40 (m, 2H), 3.16-3.07 (m, 3H), 2.91 (d, J=14.0 Hz, 1H), 2.40-2.32 (m, 1H), 2.30-2.20 (m, 1H), 1.97 (s, 3H).

The absolute stereochemistry of compounds (12A) and (12B) is arbitrarily assigned.

### Example 13

### (Z)-1⁴,1⁶-Dichloro-1¹,2¹2⁵,6¹-tetramethyl-1¹H,2¹H,6¹H-10-oxa-4,8-dithia-1(7,3)-indola-2(4,3),6(3,5)-dipyrazola-9(3,1)-naphthalenacyclotridecaphane-1²-carboxylic acid

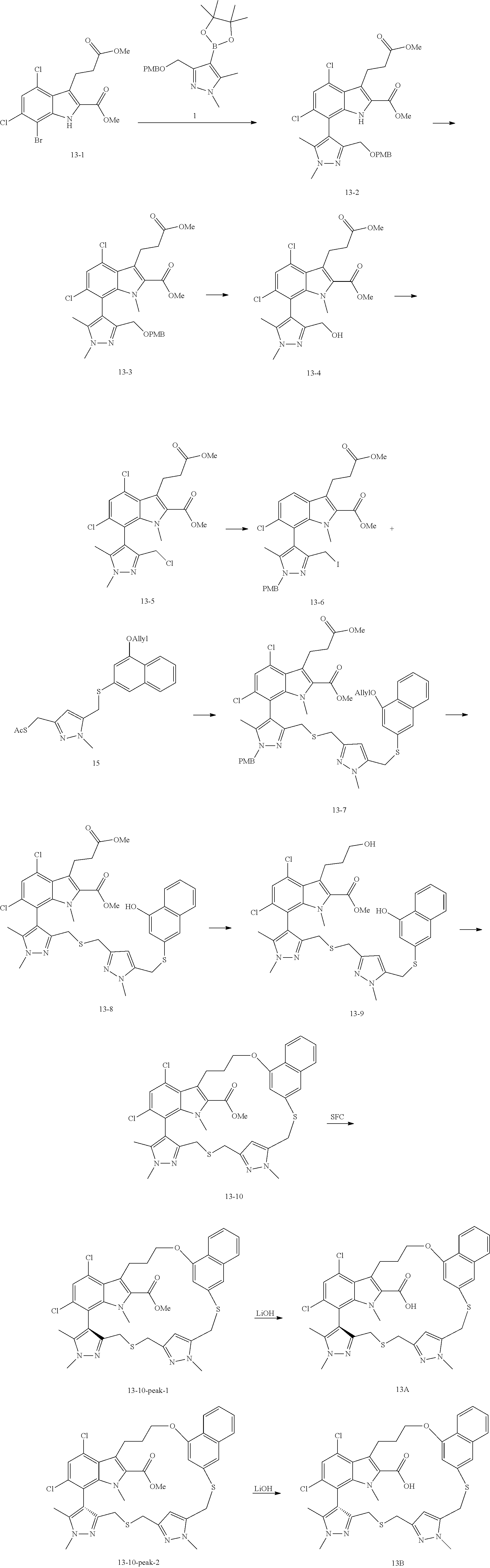

In a seal tube, a solution degassed (Ar) methyl 7-bromo-4,6-dichloro-3-(3-methoxy-3-oxopropyl)-1H-indole-2-carboxylate (13-1) prepared by the similar procedure to intermediate 14 (5×1 g, 2.46 mmol) in DMF (10 mL) were added 3-(((4-methoxybenzyl)oxy)methyl)-1,5-dimethyl-4-(4,4,5,5-tetramethyl-1,3,2-dioxaborolan-2-yl)-1H-pyrazole (1, 1.10 g, 2.95 mmol) and Cs₂CO₃ (1.20 g, 3.69 mmol). The solution was degassed with Ar and Pd(PPh₃)₄(284 mg, 0.246 mmol) was added. The solution was degassed again for 5 mins. The mixture was heated at 110° C. for 16 h. The mixture was diluted with EtOAc (50 mL) and passed through a Celite pad that was washed with EtOAc (2×50 mL). The mixture was washed with water (3×50 mL), dried over Na₂SO₄, filtered and evaporated to afford a semi pure compound that was purified by normal phase Combi purification eluting with 30-50% EtOAc in PE to afford methyl 4,6-dichloro-3-(3-methoxy-3-oxopropyl)-7-(3-(((4-methoxybenzyl)oxy)methyl)-1,5-dimethyl-1H-pyrazol-4-yl)-1H-indole-2-carboxylate (13-2, 2.0 g, 3.49 mmol, 28%) as a brown viscous liquid. MS (LCMS) m/z 574.09 [M+H]⁺. ¹H NMR (400 MHz, CDCl₃) δ 9.21 (br s, 1H), 7.25 (s, 1H), 6.98 (d, J=8.4 Hz, 2H), 6.75 (d, J=8.4 Hz, 2H), 4.37-4.33 (m, 2H), 4.15-4.09 (m, 2H), 3.89 (s, 3H), 3.78 (s, 3H), 3.77-3.67 (m, 8H), 2.75-2.70 (m, 2H), 2.08 (s, 3H).

To a stirred solution of methyl 4,6-dichloro-3-(3-methoxy-3-oxopropyl)-7-(3-(((4-methoxybenzyl)oxy)methyl)-1,5-dimethyl-1H-pyrazol-4-yl)-1H-indole-2-carboxylate (13-2, 2.7 g, 4.71 mmol) in dry DMF (27 mL) were added Cs₂CO₃ (3.84 g, 11.78 mmol) and Mel (2.20 g, 15.54 mmol), and the mixture was stirred at rt for 1 h. The reaction was quenched with water (100 mL) and extracted with EtOAc (3×100 mL). The organic layer was separated, dried over Na₂SO₄, filtered and evaporated to afford the semi pure compound that was purified by normal phase Combi purification eluting with 20-30% EtOAc in PE to afford methyl 4,6-dichloro-3-(3-methoxy-3-oxopropyl)-7-(3-(((4-methoxybenzyl)oxy)methyl)-1,5-dimethyl-1H-pyrazol-4-yl)-1-methyl-1H-indole-2-carboxylate (13-3, 2.7 g, 4.60 mmol, 97%) as a brown viscous liquid. MS (LCMS) m/z 588.31 [M+H]⁺. ¹H NMR (400 MHz, CDCl₃) δ 7.24 (s, 1H), 6.83 (d, J=8.8 Hz, 2H), 6.68 (d, J=8.4 Hz, 2H), 4.35-4.20 (m, 4H), 3.92 (s, 3H), 3.88 (s, 3H), 3.77 (s, 3H), 3.71 (s, 3H), 3.62-3.55 (m, 2H), 2.42 (s, 3H), 2.74 (t, J=7.6 Hz, 2H), 2.05 (s, 3H).

To a stirred solution of methyl 4,6-dichloro-3-(3-methoxy-3-oxopropyl)-7-(3-(((4-methoxybenzyl)oxy)methyl)-1,5-dimethyl-1H-pyrazol-4-yl)-1-methyl-1H-indole-2-carboxylate (13-3, 1.0 g, 1.70 mmol) in DCM (10 mL) was added TFA (1.94 g, 17.03 mmol) at 0° C., and the mixture was stirred at rt for 1.5 h. The reaction was quenched with a sat. aq. NaHCO₃ solution (15 mL), and extracted with DCM (3×20 mL). To the organic layer was added MeOH:TEA (1:1, 0.5 mL), and the mixture was stirred at rt for 10 min and then evaporated. The crude was portioned between water and DCM. The organic layer was separated, dried over Na₂SO₄, filtered and evaporated to give of crude 5 (900 mg). The organic layer was separated, dried over Na₂SO₄, filtered and evaporated to give the semi pure compound that was purified by normal phase GRACE purification using 60-90% EtOAc in PE to afford methyl 4,6-dichloro-7-(3-(hydroxymethyl)-1,5-dimethyl-1H-pyrazol-4-yl)-3-(3-methoxy-3-oxopropyl)-1-methyl-1H-indole-2-carboxylate (13-4, 400 mg, 0.86 mmol, 50%) as a light brown solid. MS (LCMS) m/z 468.01 [M+H]⁺. ¹H NMR (400 MHz, CDCl₃) δ 7.26 (s, 1H), 4.50-4.40 (m, 2H), 3.93 (s, 3H), 3.88 (s, 3H), 3.71 (s, 3H), 3.65-3.51 (m, 2H), 3.44 (s, 3H), 2.73 (t, J=8.0 Hz, 2H), 2.04 (s, 3H), 1.76 (t, J=6.0 Hz, 1H).

To a stirred solution of methyl 4,6-dichloro-7-(3-(hydroxymethyl)-1,5-dimethyl-1H-pyrazol-4-yl)-3-(3-methoxy-3-oxopropyl)-1-methyl-1H-indole-2-carboxylate (13-4, 1.8 g, 3.85 mmol) in DCM (20 mL) under Ar was added SOCl₂ (31 mg, 4.62 mmol) at 0° C., and the mixture was stirred at rt for 30 min. The mixture was evaporated, diluted with DCM (100 mL) and washed with a sat. NaHCO₃ solution (3×75 mL). The organic layer was separated, dried over Na₂SO₄, filtered and evaporated to afford semi pure methyl 4,6-dichloro-7-(3-(chloromethyl)-1,5-dimethyl-1H-pyrazol-4-yl)-3-(3-methoxy-3-oxopropyl)-1-methyl-1H-indole-2-carboxylate (13-5, 1.8 g, 3.71 mmol) as a light brown solid that was used without further purification. MS (LCMS) m/z 488.46 [M+H]⁺. ¹H NMR (400 MHz, CDCl₃) δ 7.28 (s, 1H), 4.47 (d, J=12.0 Hz, 1H), 4.38 (d, J=12.0 Hz, 1H), 3.94 (s, 3H), 3.90 (s, 3H), 3.72 (s, 3H), 3.66-3.51 (m, 2H), 3.47 (s, 3H), 2.74 (t, J=8.0 Hz, 2H), 2.04 (s, 3H).

To a stirred solution of methyl 4,6-dichloro-7-(3-(chloromethyl)-1,5-dimethyl-1H-pyrazol-4-yl)-3-(3-methoxy-3-oxopropyl)-1-methyl-1H-indole-2-carboxylate (13-5, 1.8 g, 3.71 mmol) in dry MeCN (20 mL) was added NaI (1.0 g, 6.68 mmol) at rt, and the mixture was heated to 80° C. for 4 h. The solvent was evaporated, and the mixture was diluted with water (100 mL) and extracted with EtOAc (3×100 mL). The organic layer was separated, dried over Na₂SO₄, filtered and evaporated to afford semi pure methyl 4,6-dichloro-7-(3-(iodomethyl)-1,5-dimethyl-1H-pyrazol-4-yl)-3-(3-methoxy-3-oxopropyl)-1-methyl-1H-indole-2-carboxylate (13-6, 1.7 g, 2.95 mmol) as a light brown solid that was used without further purification. MS (LCMS) m/z 577.97 [M+H]⁺. ¹H NMR (400 MHz, CDCl₃) δ 7.28 (s, 1H), 4.24-4.11 (m, 2H), 3.94 (s, 3H), 3.83 (s, 3H), 3.71 (s, 3H), 3.62-3.56 (m, 2H), 3.49 (s, 3H), 2.76-2.72 (m, 2H), 2.05 (s, 3H).

To a stirred solution of semi pure methyl 4,6-dichloro-7-(3-(iodomethyl)-1,5-dimethyl-1H-pyrazol-4-yl)-3-(3-methoxy-3-oxopropyl)-1-methyl-1H-indole-2-carboxylate (13-6, 1.7 g, 2.95 mmol) in MeOH (10 mL), THF (2 mL) and K₂CO₃ (2.03 g, 14.75 mmol) were added. The solution was degassed with Ar for 10 min. In another round bottom flaks, S-((5-(((4-(allyloxy)naphthalen-2-yl)thio)methyl)-1-methyl-1H-pyrazol-3-yl)methyl) ethanethioate (15, 64 mg, 2.95 mmol) in MeOH (10 mL)/THF (2 mL) was degassed with Ar for 10 min, and this solution was added to previous mixture dropwise. The mixture was stirred at rt for 16 h. The mixture was diluted with MeOH (100 mL) and filtered through Celite, The Celite was washed again with MeOH (2×50 mL), and the solvent was evaporated to give a semi pure compound that was purified by normal phase GRACE purification using 50-70% EtOAc in PE to afford methyl 7-(3-((((5-(((4-(allyloxy)naphthalen-2-yl)thio)methyl)-1-methyl-1H-pyrazol-3-yl)methyl)thio)methyl)-1,5-dimethyl-1H-pyrazol-4-yl)-4,6-dichloro-3-(3-methoxy-3-oxopropyl)-1-methyl-1H-indole-2-carboxylate (13-7, 1 g, 1.24 mmol, 42% for three steps, clean; and 1.1 g with 62% LCMS purity) as a viscous brown liquid. MS (LCMS) m/z 806.15 [M+H]⁺. ¹H NMR (400 MHz, CDCl₃) δ 8.24-8.21 (m, 1H), 7.69-7.66 (m, 1H), 7.52-7.41 (m, 2H), 7.37 (s, 1H), 7.23 (s, 1H), 6.65 (d, J=1.2 Hz, 1H), 6.19-6.09 (m, 1H), 5.93 (s, 1H), 5.52-5.46 (m, 1H), 5.34-5.29 (m, 1H), 4.68-4.60 (m, 2H), 4.06 (s, 2H), 3.91 (s, 3H), 3.83 (s, 3H), 3.70 (s, 3H), 3.69 (s, 3H), 3.61-3.41 (m, 9H), 2.72 (t, J=8.4 Hz, 2H), 2.01 (s, 3H).

To a stirred solution of methyl 7-(3-((((5-(((4-(allyloxy)naphthalen-2-yl)thio)methyl)-1-methyl-1H-pyrazol-3-yl)methyl)thio)methyl)-1,5-dimethyl-1H-pyrazol-4-yl)-4,6-dichloro-3-(3-methoxy-3-oxopropyl)-1-methyl-1H-indole-2-carboxylate (13-7, 1 g, 1.24 mmol) in DCM (10 mL) were added Pd(PPh₃)₄(29 mg, 0.025 mmol) and PhSiH₃ (161 mg, 1.49 mmol) at rt, and the mixture was stirred for 2 h. The volatiles were evaporated to give a semi pure compound that was purified by normal phase GRACE purification using 40-70% EtOAc in PE to afford methyl 4,6-dichloro-7-(3-((((5-(((4-hydroxynaphthalen-2-yl)thio)methyl)-1-methyl-1H-pyrazol-3-yl)methyl)thio)methyl)-1,5-dimethyl-1H-pyrazol-4-yl)-3-(3-methoxy-3-oxopropyl)-1-methyl-1H-indole-2-carboxylate (13-8, 800 mg, 1.046 mmol, 84%) as a viscous light brown liquid. MS (LCMS) m/z 766.12 [M+H]⁺. ¹H NMR (400 MHz, CDCl₃) δ 9.70 (br s, 1H), 8.28-8.23 (m, 1H), 7.79-7.75 (m, 1H), 7.70-7.65 (m, 2H), 7.54-7.45 (m, 1H), 7.29 (s, 1H), 6.59 (d, J=1.2 Hz, 1H), 5.99 (s, 1H), 3.93 (s, 3H), 3.92 (s, 2H), 3.88 (s, 3H), 3.72 (s, 3H), 3.63-3.42 (m, 12H), 2.78-2.74 (m, 2H), 2.05 (s, 3H).

To a suspension of methyl 4,6-dichloro-7-(3-((((5-(((4-hydroxynaphthalen-2-yl)thio)methyl)-1-methyl-1H-pyrazol-3-yl)methyl)thio)methyl)-1,5-dimethyl-1H-pyrazol-4-yl)-3-(3-methoxy-3-oxopropyl)-1-methyl-1H-indole-2-carboxylate (13-8, 2×375 mg, 0.49 mmol) in dry THF (5 mL) was added BH₃.THF (1.0 M in THF, 7.86 mL, 6.86 mmol) dropwise at 0° C. The temperature was raised to rt and stirred for 6 h. The reaction was quenched with MeOH (5 mL) and 6N HCl (5 mL) at 0° C. The mixture was stirred at 0° C. for 10 min and then at rt for 20 min. The mixture was diluted with water (5 mL) and extracted with 10% MeOH in DCM (3×20 mL). The organic layer was separated, washed with sat. NaHCO₃ (3×20 mL), dried over Na₂SO₄, filtered and evaporated to give the semi pure compound that was purified by normal phase Combi purification along with another 250 mg batch using 50-80% EtOAc in PE to afford methyl 4,6-dichloro-7-(3-((((5-(((4-hydroxynaphthalen-2-yl)thio)methyl)-1-methyl-1H-pyrazol-3-yl)methyl)thio)methyl)-1,5-dimethyl-1H-pyrazol-4-yl)-3-(3-hydroxypropyl)-1-methyl-1H-indole-2-carboxylate (13-9, 650 mg, 0.88 mmol, 67%, altogether for 1 g batch) as a light brown solid. MS (LCMS) m/z 738.12 [M+H]⁺. ¹H NMR (400 MHz, CDCl₃) δ 9.60 (br s, 1H), 8.26-8.23 (m, 1H), 7.76-7.74 (m, 1H), 7.65 (s, 1H), 7.52-7.44 (m, 2H), 7.29 (s, 1H), 6.60 (d, J=1.6 Hz, 1H), 5.99 (s, 1H), 3.94 (s, 3H), 3.93 (s, 2H), 3.92-3.90 (m, 2H), 3.89 (s, 3H), 3.78-3.70 (m, 2H), 3.61-3.32 (m, 10H), 2.09 (s, 3H), 1.80-1.50 (m, 2H).

To a stirred solution of TPP (178 mg, 0.68 mmol) in toluene (5 mL) was added a solution of di-tert-butyl diazene-1,2-dicarboxylate (157 mg, 0.68 mmol), methyl 4,6-dichloro-7-(3-((((5-(((4-hydroxynaphthalen-2-yl)thio)methyl)-1-methyl-1H-pyrazol-3-yl)methyl)thio)methyl)-1,5-dimethyl-1H-pyrazol-4-yl)-3-(3-hydroxypropyl)-1-methyl-1H-indole-2-carboxylate (13-9, 2×250 mg, 0.34 mmol) in toluene (5 mL) and THF (1.0 mL), and the mixture was stirred at rt for 1 h. The reaction was quenched with water (10 mL) and extracted with EtOAc (3×15 mL). The organic layer was dried over Na₂SO₄, filtered, concentrated to give the semi pure compound that was purified by RP prep HPLC and fractions were evaporated under reduced pressure to afford methyl (Z)-1⁴,1⁶-dichloro-1¹,2¹,2⁵,6¹-tetramethyl-1¹H,2¹H,6¹H-10-oxa-4,8-dithia-1(7,3)-indola-2(4,3),6(3,5)-dipyrazola-9(3,1)-naphthalenacyclotridecaphane-1²-carboxylate (13-10, 110 mg, 0.15 mmol, 22% for two batches) as an off white solid. MS (LCMS) m/z 720.13 [M+H]⁺. ¹H NMR (400 MHz, CDCl₃) δ 8.20-8.17 (m, 1H), 7.78-7.63 (m, 1H), 7.47-7.42 (m, 3H), 7.27 (s, 1H), 6.43 (d, J=1.2 Hz, 1H), 5.31 (s, 1H), 4.05-3.90 (m, 4H), 3.85 (s, 3H), 3.73 (s, 3H), 3.66 (s, 3H), 3.52 (s, 3H), 3.52-3.48 (m, 1H), 3.20 (d, J=13.6 Hz, 1H), 3.07 (s, 2H), 2.40-2.30 (m, 2H), 2.06 (s, 3H), 1.35-1.20 (m, 2H).

Methyl (Z)-1⁴,1⁶-dichloro-1¹,2¹,2⁵,6¹-tetramethyl-1¹H,2¹H,6¹H-10-oxa-4,8-dithia-1(7,3)-indola-2(4,3),6(3,5)-dipyrazola-9(3,1)-naphthalenacyclotridecaphane-1²-carboxylate (13-10, 110 mg, LCMS 98%) was purified by SFC purification to afford 13-10-peak-1 (21 mg) as an off white solid with 81.8% of LCMS purity (chiral HPLC:99.80%) and 13-10-peak-2 (15 mg) as an off white solid with 95.9% of LCMS purity (chiral HPLC:99.90%). These two peaks were separately used for the next steps to get respective final compound.

13-10-peak-1: MS (LCMS) m/z 720.48 [M+H]⁺.

13-10-peak-2: MS (LCMS) m/z 720.48 [M+H]⁺.

To a stirred solution of methyl (Z)-1⁴,1⁶-dichloro-1¹,2¹,2⁵,6¹-tetramethyl-1H,2¹H,6¹H-10-oxa-4,8-dithia-1(7,3)-indola-2(4,3),6(3,5)-dipyrazola-9(3,1)-naphthalenacyclotridecaphane-1²-carboxylate (13-10-Peak-1, 21 mg, 0.029 mmol) in MeOH:THF:H₂O (10:10:3, 1 mL) was added a solution of LiOH.H₂O (18.5 mg, 0.44 mmol) at 0° C., and the mixture was stirred at rt for 2 h. Another portion of LiOH.H₂O (9.0 mg, 0.22 mmol) was added at 0° C., and the mixture was stirred at rt for 1 h. The solvent was evaporated, and the mixture was diluted with water (2 mL). The aqueous layer was acidified to pH 2 using 1N aq. HCl, and the mixture was stirred for 1 min. A solid precipitation was observed that was filtered, washed with water and dried to get (Z)-1⁴,1⁶-dichloro-1¹,2¹,2⁵,6¹-tetramethyl-1¹H,2¹H,6¹H-10-oxa-4,8-dithia-1(7,3)-indola-2(4,3),6(3,5)-dipyrazola-9(3,1)-naphthalenacyclotridecaphane-1²-carboxylic acid (13A, 18 mg, 0.0255 mmol, 88%) as an off white solid. MS (LCMS) m/z 706.12 [M+H]⁺. ¹H NMR (400 MHz, DMSO-d₆) δ 7.98 (d, J=8.0 Hz, 1H), 7.63 (d, J=8.0 Hz, 1H), 7.44-7.33 (m, 2H), 7.24 (s, 1H), 7.15 (s, 1H), 7.07 (s, 1H), 5.85 (s, 1H), 4.97 (d, J=15.6 Hz, 1H), 4.20-4.04 (m, 3H), 3.75 (s, 3H), 3.74 (s, 3H), 3.46 (d, J=14.0 Hz, 1H), 3.40-3.35 (m, 1H), 3.30 (s, 3H), 3.20-3.10 (m, 4H), 2.32-2.15 (m, 2H), 2.00 (s, 3H).

To a stirred solution of methyl (Z)-1⁴,1⁶-dichloro-1¹,2¹,2⁵,6¹-tetramethyl-1¹H,2¹H,6¹H-10-oxa-4,8-dithia-1(7,3)-indola-2(4,3),6(3,5)-dipyrazola-9(3,1)-naphthalenacyclotridecaphane-1²-carboxylate (13-10-peak-2, 30 mg, 0.042 mmol) in MeOH:THF:H₂O (10:10:3, 1 mL) was added a solution of LiOH.H₂O (26.3 mg, 0.63 mmol) at 0° C., and the mixture was stirred at rt for 2 h. Another portion of LiOH.H₂O (13.0 mg, 0.31 mmol) was added at 0° C., and the mixture was stirred at rt for 1 h. The solvent was evaporated, and the mixture was diluted with water (2 mL). The aqueous layer was acidified to pH 2 using 1N aq. HCl, and the mixture was stirred for 1 min. A solid precipitation was observed that was filtered, washed with water and dried to get (Z)-1⁴,1⁶-dichloro-1¹,2¹,2⁵,6¹-tetramethyl-1¹H,2¹H,6¹H-10-oxa-4,8-dithia-1(7,3)-indola-2(4,3),6(3,5)-dipyrazola-9(3,1)-naphthalenacyclotridecaphane-1²-carboxylic acid (13B, 27 mg, 0.038 mmol, 91%) as an off white solid. MS (LCMS) m/z 706.12 [M+H]⁺. ¹H NMR (400 MHz, DMSO-d₆) δ 7.98 (d, J=8.4 Hz, 1H), 7.64 (d, J=8.0 Hz, 1H), 7.45-7.34 (m, 2H), 7.26 (s, 1H), 7.18 (s, 1H), 7.00 (s, 1H), 5.76 (s, 1H), 4.97 (d, J=15.6 Hz, 1H), 4.20-4.01 (m, 3H), 3.75 (s, 3H), 3.74 (s, 3H), 3.45 (d, J=13.6 Hz, 1H), 3.40-3.35 (m, 1H), 3.31 (s, 3H), 3.20-3.10 (m, 4H), 2.32-2.15 (m, 2H), 2.00 (s, 3H).

The absolute stereochemistry of compounds (13A) and (13B) is arbitrarily assigned.

### Example 14

### (Z)-1⁶-Fluoro-1¹,2¹,2⁵,6¹-tetramethyl-1¹H,2¹H,6¹H-10-oxa-4,8-dithia-1(7,3)-indola-2(4,3),6(3,5)-dipyrazola-9(3,1)-naphthalenacyclotridecaphane-1²-carboxylic acid

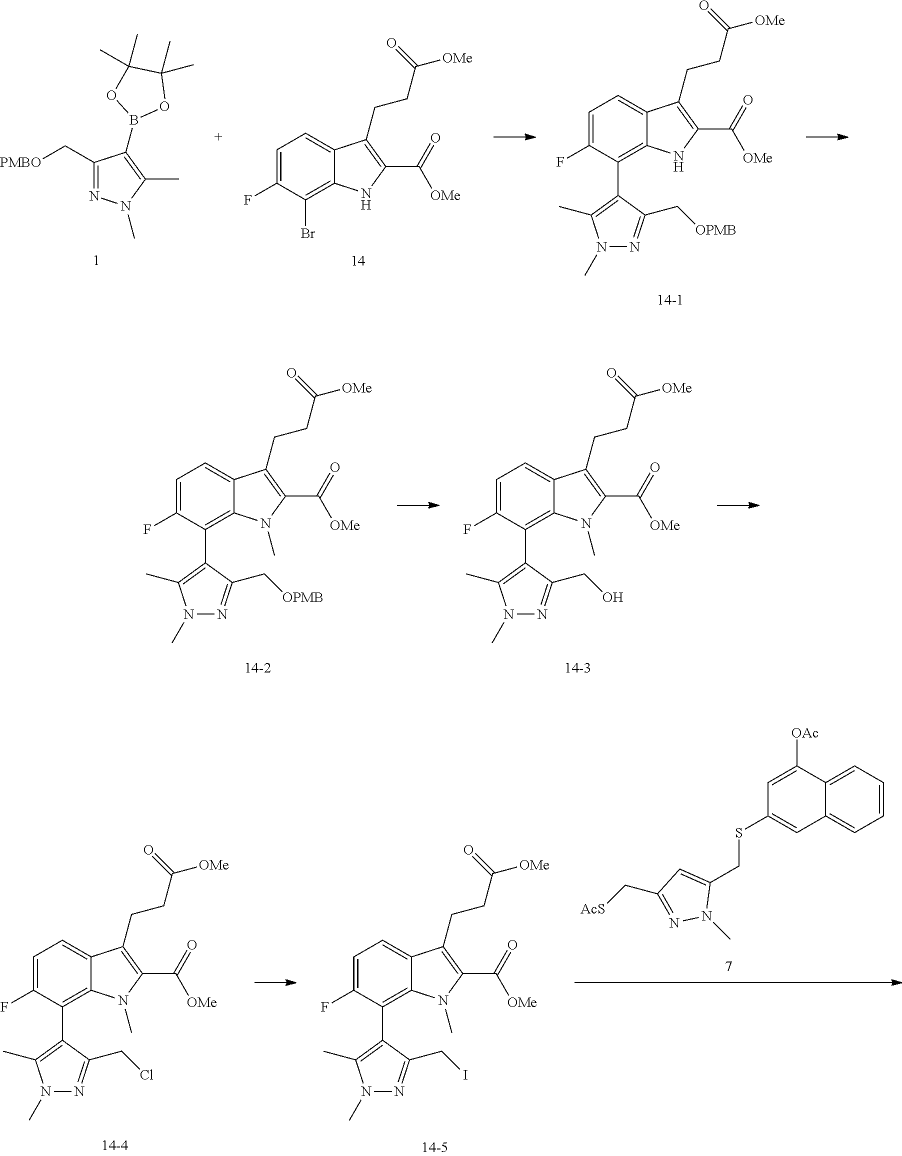

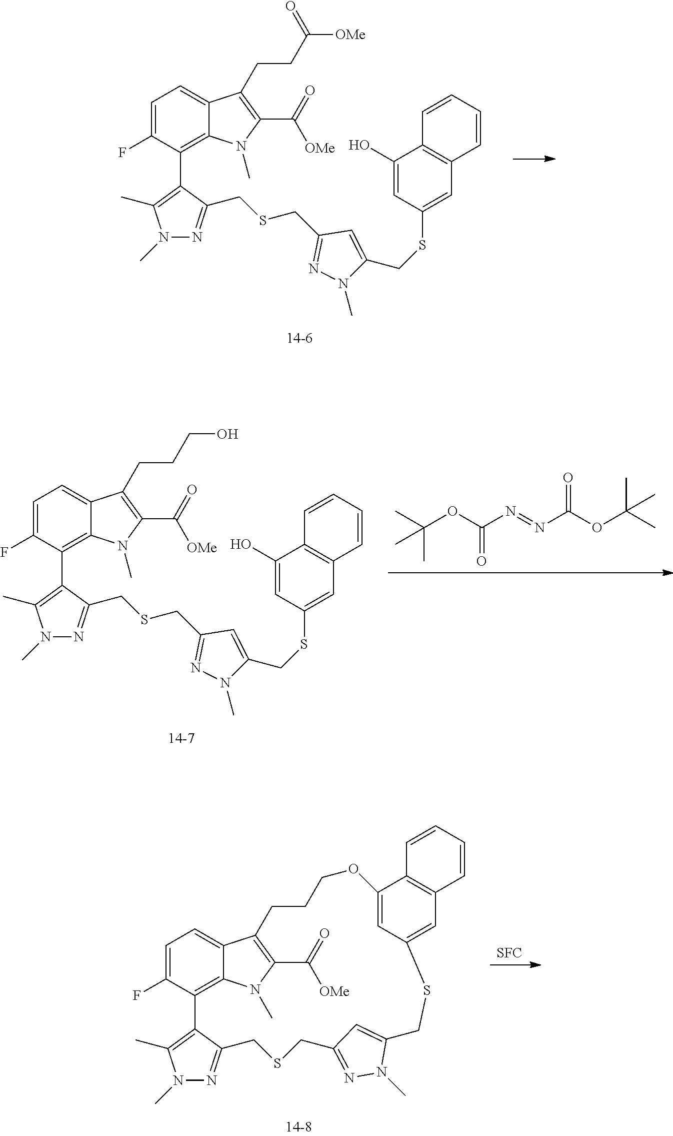

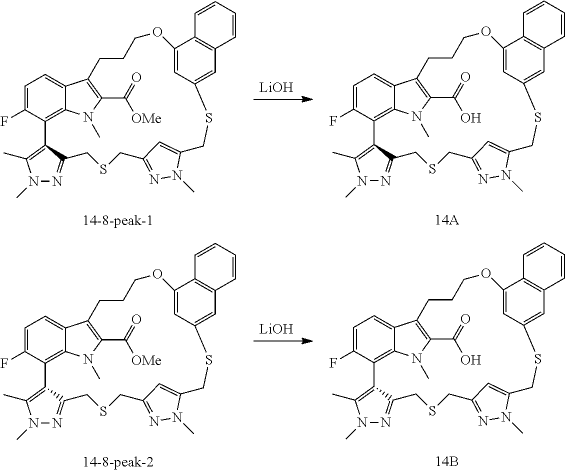

In a round bottom flask, to an Ar degassed solution of methyl 7-bromo-6-fluoro-3-(3-methoxy-3-oxopropyl)-1H-indole-2-carboxylate (14, 6×5 g, 14.005 mmol) in dioxane (100 mL) were added water (25 mL), 3-(((4-methoxybenzyl)oxy)methyl)-1,5-dimethyl-4-(4,4,5,5-tetramethyl-1,3,2-dioxaborolan-2-yl)-1H-pyrazole (1) (6.512 g, 17.507 mmol), Cs₂CO₃ (9.13 g, 28.011 mmol) and PdCl₂(dtbpf) (273 mg, 4.201 mmol). The mixture was heated at 100° C. for 3 h. The mixture was diluted with EtOAc (50 mL) and passed through a Celite pad that was washed with EtOAc (2×50 mL). The mixture was washed with water (3×50 mL), dried over Na₂SO₄, filtered and evaporated to afford the semi pure compound that was purified by silica gel (100-200) eluting with 70% EtOAc in PE to afford methyl 6-fluoro-3-(3-methoxy-3-oxopropyl)-7-(3-(((4-methoxybenzyl)oxy)methyl)-1,5-dimethyl-1H-pyrazol-4-yl)-1H-indole-2-carboxylate (14-1, 28 g, 53.537 mmol, 63%) as a brown sticky liquid. MS (LCMS) m/z 524.53 [M+H]⁺. ¹H NMR (400 MHz, CDCl₃) δ 9.82 (br s, 1H), 7.67-7.63 (m, 1H), 7.13 (d, J=6.4 Hz, 2H), 7.02-6.97 (m, 1H), 6.81-6.78 (m, 2H), 4.47-4.37 (m, 2H), 4.17-4.09 (m, 2H), 3.89 (s, 3H), 3.78 (s, 3H), 3.72 (s, 3H), 3.66 (s, 3H), 3.45-3.40 (m, 2H), 2.74-2.70 (m, 2H) 2.15 (s, 3H).

To a stirred solution of methyl 6-fluoro-3-(3-methoxy-3-oxopropyl)-7-(3-(((4-methoxybenzyl)oxy)methyl)-1,5-dimethyl-1H-pyrazol-4-yl)-1H-indole-2-carboxylate (14-1, 28 g, 53.537 mmol) in dry DMF (280 mL) were added Cs₂CO₃ (26.2 g, 80.305 mmol) and Mel (6.66 mL, 107.07 mmol) at 0° C., and the mixture was stirred at rt for 1 h. The reaction was quenched with water (300 mL) and extracted with EtOAc (2×300 mL). The organic layer was separated, dried over Na₂SO₄, filtered and evaporated to afford methyl 6-fluoro-3-(3-methoxy-3-oxopropyl)-7-(3-(((4-methoxybenzyl)oxy)methyl)-1,5-dimethyl-1H-pyrazol-4-yl)-1-methyl-1H-indole-2-carboxylate (14-2, 26 g, 48.417 mmol, 90%) as a brown viscous liquid. ¹H NMR (400 MHz, CDCl₃) δ 7.66-7.66 (m, 1H), 6.96 (t, J=9.2 Hz, 1H), 6.90-6.85 (m, 2H), 6.69-6.66 (m, 2H), 4.47 (d, J=15.2 Hz, 1H), 4.35-4.32 (m, 2H), 4.24 (d, J=11.6 Hz, 1H), 3.90 (s, 3H), 3.88 (s, 3H), 3.74 (s, 3H), 3.67 (s, 3H), 3.53 (s, 3H), 3.38-3.33 (m, 2H), 2.67 (t, J=8 Hz, 2H), 2.10 (s, 3H).

To a stirred solution of methyl 6-fluoro-3-(3-methoxy-3-oxopropyl)-7-(3-(((4-methoxybenzyl)oxy)methyl)-1,5-dimethyl-1H-pyrazol-4-yl)-1-methyl-1H-indole-2-carboxylate (14-2, 26 g, 49.713 mmol) in DCM (78 mL) was added TFA (38 mL, 497.1 mmol) at 0° C., and the mixture was stirred at rt for 1 h. The solvent was evaporated. The reaction was quenched with a sat. aq. NaHCO₃ solution (200 mL) and extracted with DCM (3×200 mL). To the organic layer was added MeOH:TEA (1:1, 10 mL). The mixture was stirred at rt for 10 min and then evaporated. The mixture was washed with brine. The organic layer was separated, dried over Na₂SO₄, filtered and evaporated. The crude was purified by silica gel (100-200) using 10% MeOH in DCM to afford methyl 6-fluoro-7-(3-(hydroxymethyl)-1,5-dimethyl-1H-pyrazol-4-yl)-3-(3-methoxy-3-oxopropyl)-1-methyl-1H-indole-2-carboxylate (14-3, 14 g, 33.573 mmol, 67%) as an off white solid. ¹H NMR (400 MHz, CDCl₃) δ 7.66 (dd, J=8.8, 5.6 Hz, 1H), 6.98 (t, J=9.6 Hz, 1H), 4.50 (d, J=2.4 Hz, 1H), 3.92 (s, 3H), 3.89 (s, 3H), 3.68 (s, 3H), 3.57 (s, 3H), 3.39-3.32 (m, 2H), 2.67 (t, J=4.8 Hz, 2H), 2.11 (s, 3H).

To a stirred solution of methyl 6-fluoro-7-(3-(hydroxymethyl)-1,5-dimethyl-1H-pyrazol-4-yl)-3-(3-methoxy-3-oxopropyl)-1-methyl-1H-indole-2-carboxylate (14-3, 10 g, 23.98 mmol) in DCM (120 mL) was added SOCl₂ (2.07 mL, 28.77 mmol) at 0° C., and the mixture was stirred at rt for 30 min. The solvent was evaporated. The mixture was diluted with DCM (200 mL) and washed with a sat. NaHCO₃ solution (100 mL). The organic layer was separated, dried over Na₂SO₄, filtered and evaporated to afford semi pure methyl 7-(3-(chloromethyl)-1,5-dimethyl-1H-pyrazol-4-yl)-6-fluoro-3-(3-methoxy-3-oxopropyl)-1-methyl-1H-indole-2-carboxylate (14-4, 10 g, 22.98 mmol) as a brown sticky liquid that was used without further purification. ¹H NMR (400 MHz, CDCl₃) δ 7.68 (dd, J=8.8, 5.6 Hz, 1H), 6.99 (t, J=8.8 Hz, 1H), 4.46 (d, J=3.2 Hz, 2H), 3.92 (s, 3H), 3.89 (s, 3H), 3.67 (s, 3H), 3.58 (s, 3H), 3.39-3.31 (m, 2H), 2.69-2.65 (m, 2H), 2.09 (s, 3H).

To a stirred solution of semi pure methyl 7-(3-(chloromethyl)-1,5-dimethyl-1H-pyrazol-4-yl)-6-fluoro-3-(3-methoxy-3-oxopropyl)-1-methyl-1H-indole-2-carboxylate (14-4, 10 g, 22.988 mmol) in dry MeCN (100 mL) was added NaI (6.2 g, 41.379 mmol) at rt, and the mixture was heated to 80° C. for 2.5 h. The solvent was evaporated. The mixture was diluted with water (100 mL) and extracted with EtOAc (2×100 mL). The organic layer was separated, dried over Na₂SO₄, filtered and evaporated to afford semi pure methyl 6-fluoro-7-(3-(iodomethyl)-1,5-dimethyl-1H-pyrazol-4-yl)-3-(3-methoxy-3-oxopropyl)-1-methyl-1H-indole-2-carboxylate (14-5, 11 g, 20.872 mmol) as a light brown sticky solid that was used without further purification. MS (LCMS) m/z 528.21 [M+H]⁺. ¹H NMR (400 MHz, CDCl₃) δ 7.69 (dd, J=8.8, 5.2 Hz, 1H), 7.00 (t, J=9.2 Hz, 1H), 4.25 (s, 2H), 3.93 (s, 3H), 3.86 (s, 3H), 3.67 (s, 3H), 3.60 (s, 3H), 3.39-3.34 (m, 3H), 2.68 (t, J=8 Hz, 1H), 2.08 (s, 3H).

To a stirred solution of semi pure methyl 6-fluoro-7-(3-(iodomethyl)-1,5-dimethyl-1H-pyrazol-4-yl)-3-(3-methoxy-3-oxopropyl)-1-methyl-1H-indole-2-carboxylate (14-5, 11 g, 20.872 mmol) in MeOH (33 mL), THF (10 mL) and K₂CO₃ (3.74 g, 27.134 mmol) were added. The solution was degassed with Ar for 10 min. In another round bottom flask, 3-(((3-((acetylthio)methyl)-1-methyl-1H-pyrazol-5-yl)methyl)thio)naphthalen-1-yl acetate (7, 9.18 g, 22.96 mmol) in MeOH (33 mL) and THF (10 mL) was degassed with Ar for 10 min, and this solution was added to previous mixture dropwise. The mixture was stirred at rt for 16 h. The solvent was evaporated. The mixture was diluted with water (100 mL) and extracted with EtOAc (2×100 mL). The organic layer was separated, dried over Na₂SO₄, filtered and evaporated. The crude was purified by silica gel (100-200) using 80% EtOAc in PE to afford methyl 6-fluoro-7-(3-((((5-(((4-hydroxynaphthalen-2-yl)thio)methyl)-1-methyl-1H-pyrazol-3-yl)methyl)thio)methyl)-1,5-dimethyl-1H-pyrazol-4-yl)-3-(3-methoxy-3-oxopropyl)-1-methyl-1H-indole-2-carboxylate (14-6, 10 g, 13.986 mmol, 58% for three steps) as an off white foamy solid. MS (LCMS) m/z 716.39 [M+H]⁺. ¹H NMR (400 MHz, CDCl₃) δ 9.9 (s, 1H), 8.29-8.27 (m, 1H), 7.76-7.74 (m, 1H), 7.69-7.65 (m, 2H), 7.52-7.45 (m, 2H), 6.99 (t, J=9.6 Hz, 1H), 6.02 (d, J=1.6 Hz, 1H), 5.96 (s, 1H), 3.93 (s, 3H), 3.91 (s, 2H), 3.86 (s, 3H), 3.69 (s, 3H), 3.62 (s, 3H), 3.57-3.54 (m, 2H), 3.49 (s, 3H), 3.47 (d, J=7.6 Hz, 2H), 3.41-3.35 (m, 2H), 2.69 (t, J=8.4 Hz, 2H), 2.10 (s, 3H).

To a suspension of methyl 6-fluoro-7-(3-((((5-(((4-hydroxynaphthalen-2-yl)thio)methyl)-1-methyl-1H-pyrazol-3-yl)methyl)thio)methyl)-1,5-dimethyl-1H-pyrazol-4-yl)-3-(3-methoxy-3-oxopropyl)-1-methyl-1H-indole-2-carboxylate (14-6, 10 g, 13.986 mmol) in dry THF (100 mL) was added BH₃.THF (1.0 M in THF, 77 mL, 76.92 mmol) dropwise at 0° C., and the mixture was stirred at rt for 16 h. The reaction was quenched with MeOH (77 mL) and 6N HCl (77 mL) at 0° C., and the mixture was stirred at rt for 20 min. The mixture was further diluted with water (50 mL) and extracted with 10% MeOH in DCM (2×50 mL). The organic layer was separated and evaporated. The crude was dissolved in 10% MeOH in DCM (200 mL), washed with sat. NaHCO₃ (2×100 mL), dried over Na₂SO₄, filtered and evaporated to give the semi pure compound that was purified by silica gel column (100-200) purification using 100% EtOAc to afford methyl 6-fluoro-7-(3-((((5-(((4-hydroxynaphthalen-2-yl)thio)methyl)-1-methyl-1H-pyrazol-3-yl)methyl)thio)methyl)-1,5-dimethyl-1H-pyrazol-4-yl)-3-(3-hydroxypropyl)-1-methyl-1H-indole-2-carboxylate (14-7, 4.05 g, 5.89 mmol, 42%) as an off white foamy solid. MS (LCMS) m/z 688.66 [M+H]⁺. ¹H NMR (300 MHz, DMSO) δ 10.25 (br s, 1H), 8.04 (d, J=10.8 Hz, 1H), 7.74-7.69 (m, 1H), 7.47-7.00 (m, 6H), 6.79 (d, J=2 Hz, 1H), 5.91 (s, 1H), 4.47 (br s, 1H), 4.27 (s, 2H), 3.83 (s, 3H), 3.77 (s, 3H), 3.67 (s, 3H), 3.45 (s, 5H), 3.38 (d, J=8 Hz, 4H), 3.02-2.98 (m, 2H), 2.01 (s, 3H), 1.75-1.70 (m, 2H).

To a stirred solution of TPP (763 mg, 2.911 mmol) in toluene (14 mL) was added a solution of di-tert-butyl diazene-1,2-dicarboxylate (669 mg, 2.911 mmol), methyl 6-fluoro-7-(3-((((5-(((4-hydroxynaphthalen-2-yl)thio)methyl)-1-methyl-1H-pyrazol-3-yl)methyl)thio)methyl)-1,5-dimethyl-1H-pyrazol-4-yl)-3-(3-hydroxypropyl)-1-methyl-1H-indole-2-carboxylate (14-7, 2×1 g, 1.455 mmol) in toluene (14 mL) and THF (2 mL). The mixture was stirred at rt for 2 h. The reaction was quenched with water (50 mL) and extracted with EtOAc (2×50 mL). The organic layer was dried over Na₂SO₄, filtered and concentrated. The crude was triturated with MeOH to afford methyl (Z)-1⁶-fluoro-1¹,2¹,2⁵,6¹-tetramethyl-1¹H,2¹H,6¹H-10-oxa-4,8-dithia-1(7,3)-indola-2(4,3),6(3,5)-dipyrazola-9(3,1)-naphthalenacyclotridecaphane-1²-carboxylate (14-8, 650 mg, 0.97 mmol, 33% for two batches) as an off white solid. MS (LCMS) m/z 670.62 [M+H]⁺.

Methyl (Z)-1⁶-fluoro-1¹,2¹,2⁵,6¹-tetramethyl-1¹H,2¹H,6¹H-10-oxa-4,8-dithia-1(7,3)-indola-2(4,3),6(3,5)-dipyrazola-9(3,1)-naphthalenacyclotridecaphane-1²-carboxylate (14-8, 252 mg, LCMS 90%) was purified by SFC purification to afford 14-8-peak-1 (93 mg) as an off white solid and 14-8-peak-2 (79 mg) as an off white solid. These two peaks were separately used for the next steps to get respective final compound.

To a nitrogen degassed solution of methyl (Z)-1⁶-fluoro-1¹,2¹,2⁵,6¹-tetramethyl-1¹H,2¹H,6¹H-10-oxa-4,8-dithia-1(7,3)-indola-2(4,3),6(3,5)-dipyrazola-9(3,1)-naphthalenacyclotridecaphane-1²-carboxylate (14-8-peak-1, 93 mg, 0.139 mmol) in MeOH:THF:H₂O (1:1:1, 1 mL) was added LiOH.H₂O (87.6 mg, 2.085 mmol) at rt, and the mixture was stirred at 80° C. for 3 h. The solvent was evaporated. The mixture was diluted with water (1 mL), acidified to pH 2 using 1N aq. HCl and filtered to afford (Z)-1⁶-fluoro-1¹,2¹,2⁵,6¹-tetramethyl-1¹H,2¹H,6¹H-10-oxa-4,8-dithia-1(7,3)-indola-2(4,3),6(3,5)-dipyrazola-9(3,1)-naphthalenacyclotridecaphane-1²-carboxylic acid (14A, 86 mg, 0.131 mmol, 94%) as an off white solid. MS (LCMS) m/z 656.35 [M+H]⁺. ¹H NMR (400 MHz, DMSO-d₆) δ 13.2 (br s, 1H), 8.09 (d, J=8.0 Hz, 1H), 7.92-7.89 (m, 1H), 7.71 (d, J=7.6 Hz, 1H), 7.50-7.43 (m, 2H), 7.36 (s, 1H), 6.92 (t, J=9.2 Hz, 1H), 6.71 (s, 1H), 4.76 (s, 1H), 4.28-4.23 (m, 2H), 4.20-4.13 (m, 1H), 3.86-3.79 (m, 1H), 3.75 (s, 3H), 3.71 (s, 3H), 3.54 (s, 3H), 3.48-3.40 (m, 2H), 3.21-3.08 (m, 3H), 2.92 (d, J=14.4 Hz, 1H), 2.50-2.32 (m, 2H), 2.01 (s, 3H).

To a nitrogen degassed solution of methyl (Z)-1⁶-fluoro-1¹,2¹,2⁵,6¹-tetramethyl-1¹H,2¹H,6¹H-10-oxa-4,8-dithia-1(7,3)-indola-2(4,3),6(3,5)-dipyrazola-9(3,1)-naphthalenacyclotridecaphane-1²-carboxylate (14-8-peak-2, 79 mg, 0.118 mmol) in MeOH:THF:H₂O (1:1:1, 1 mL) was added LiOH.H₂O (74.4 mg, 1.771 mmol) at rt, and the mixture was stirred at 80° C. for 3 h. The solvent was evaporated. The mixture was diluted with water (1 mL), acidified to pH 2 using 1N aq. HCl and filtered to afford (Z)-1⁶-fluoro-1¹,2¹,2⁵,6¹-tetramethyl-1¹H,2¹H,6¹H-10-oxa-4,8-dithia-1(7,3)-indola-2(4,3),6(3,5)-dipyrazola-9(3,1)-naphthalenacyclotridecaphane-1²-carboxylic acid (14B, 74 mg, 0.113 mmol, 96%) as an off white solid. MS (LCMS) m/z 656.35 [M+H]⁺. ¹H NMR (400 MHz, DMSO-d₆) δ 13.2 (br s, 1H), 8.09 (d, J=8.0 Hz, 1H), 7.92-7.89 (m, 1H), 7.71 (d, J=7.6 Hz, 1H), 7.50-7.43 (m, 2H), 7.36 (s, 1H), 6.92 (t, J=9.2 Hz, 1H), 6.71 (s, 1H), 4.76 (s, 1H), 4.32-4.23 (m, 2H), 4.18-4.13 (m, 1H), 3.86-3.84 (m, 1H), 3.75 (s, 3H), 3.71 (s, 3H), 3.54 (s, 3H), 3.48-3.40 (m, 2H), 3.14-3.08 (m, 3H), 2.92 (d, J=14.4 Hz, 1H), 2.38-2.24 (m, 2H), 2.01 (s, 3H).

The absolute stereochemistry of compounds (14A) and (14B) is arbitrarily assigned.

### Example 15

### (Z)-6¹-Ethyl-1⁶-fluoro-1¹,2¹,2³-trimethyl-1¹H,2¹H,6¹H-10-oxa-4,8-dithia-1(7,3)-indola-2(4,5),6(3,5)-dipyrazola-9(3,1)-naphthalenacyclotridecaphane-1²-carboxylic acid

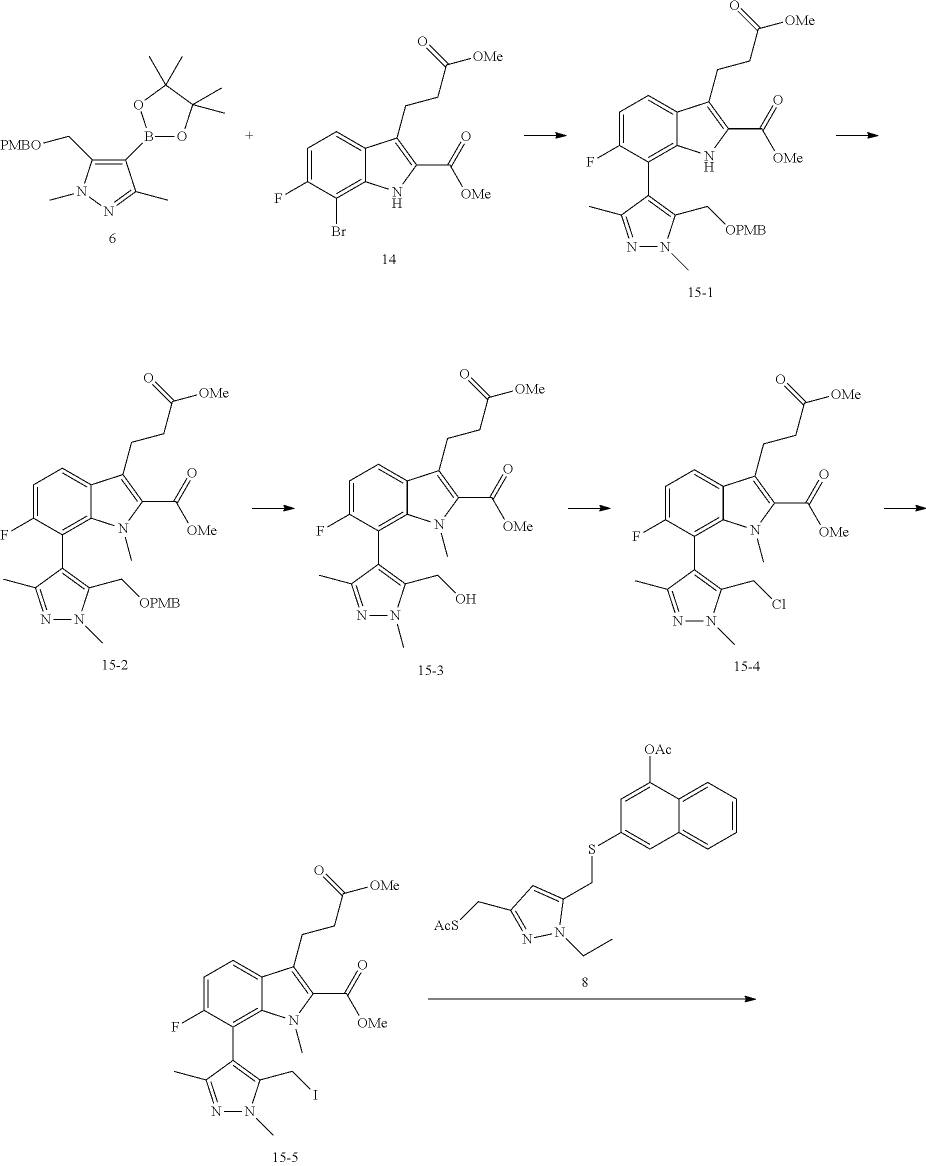

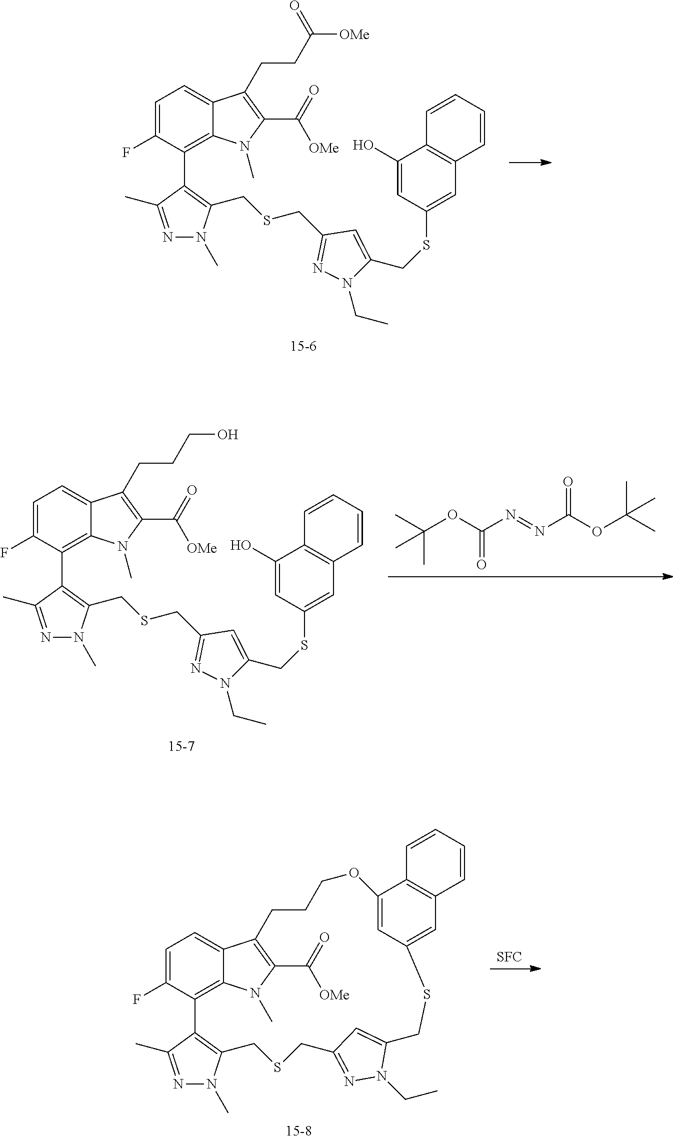

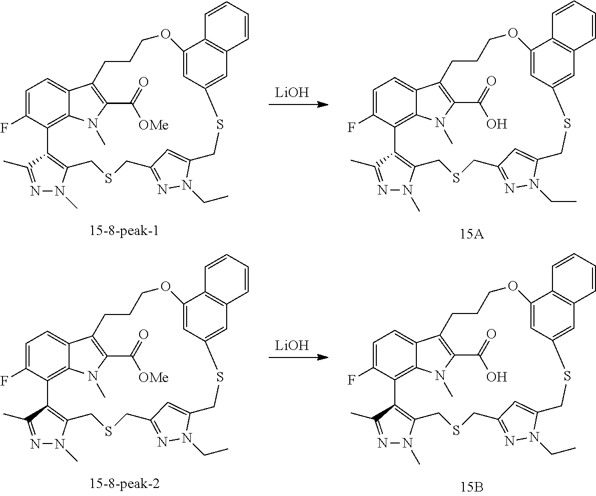

In a round bottom flaks, to an Ar degassed solution of 5-(((4-methoxybenzyl)oxy)methyl)-1,3-dimethyl-4-(4,4,5,5-tetramethyl-1,3,2-dioxaborolan-2-yl)-1H-pyrazole (6, 3×5 g, 13.440 mmol) in dioxane (75 mL) were added methyl 7-bromo-6-fluoro-3-(3-methoxy-3-oxopropyl)-1H-indole-2-carboxylate (14, 3.3 g, 9.408 mmol), Cs₂CO₃ (8.7 g, 26.881 mmol) and Pd(PPh₃)₄(1.55 g, 1.344 mmol). The mixture was heated at 100° C. for 16 h. The mixture was diluted with EtOAc (50 mL) and passed through a Celite pad that was washed with EtOAc (2×50 mL). The filtrate was evaporated to afford semi pure compound 7 that was purified by silica gel (100-200) eluting with 25-30% EtOAc in PE to afford methyl 6-fluoro-3-(3-methoxy-3-oxopropyl)-7-(5-(((4-methoxybenzyl)oxy)methyl)-1,3-dimethyl-1H-pyrazol-4-yl)-1H-indole-2-carboxylate (15-1, 7 g, 13.384 mmol, 33%) as a brown sticky liquid. MS (LCMS) m/z 524.56 [M+H]⁺. ¹H NMR (400 MHz, CDCl₃) δ 9.30 (s, 1H), 7.67-7.64 (m, 1H), 7.15 (dd, J=6.4 Hz, 2 Hz, 2H), 7.03-6.99 (m, 1H), 6.85-6.82 (m, 2H), 4.65-4.53 (m, 1H), 4.31-4.24 (m, 2H), 4.14-4.09 (m, 1H), 3.82 (s, 3H), 3.81 (s, 3H), 3.80 (s, 3H), 3.67 (s, 3H), 3.45-3.40 (m, 2H), 2.73 (t, J=7.6 Hz, 2H), 2.15 (s, 3H).

To a stirred solution of methyl 6-fluoro-3-(3-methoxy-3-oxopropyl)-7-(5-(((4-methoxybenzyl)oxy)methyl)-1,3-dimethyl-1H-pyrazol-4-yl)-1H-indole-2-carboxylate (15-1, 5 g, 9.56 mmol) in dry DMF (50 mL) were added Cs₂CO₃ (4.67 g, 14.34 mmol) and Mel (1.19 mL, 19.12 mmol) at 0° C., and the mixture was stirred at rt for 1 h. The reaction was quenched with water (300 mL) and extracted with EtOAc (2×200 mL). The organic layer was separated, dried over Na₂SO₄, filtered and evaporated to afford methyl 6-fluoro-3-(3-methoxy-3-oxopropyl)-7-(5-(((4-methoxybenzyl)oxy)methyl)-1,3-dimethyl-1H-pyrazol-4-yl)-1-methyl-1H-indole-2-carboxylate (15-2, 5 g, 9.31 mmol, 98%) as a brown viscous liquid. MS (LCMS) m/z 538.96 [M+H]⁺.

To a stirred solution of methyl 6-fluoro-3-(3-methoxy-3-oxopropyl)-7-(5-(((4-methoxybenzyl)oxy)methyl)-1,3-dimethyl-1H-pyrazol-4-yl)-1-methyl-1H-indole-2-carboxylate (15-2, 5 g, 9.310 mmol) in DCM (50 mL) was added TFA (7.12 mL, 93.109 mmol) at 0° C., and the mixture was stirred at rt for 1.5 h. The solvent was evaporated. The reaction was quenched with a sat. aq. NaHCO₃ solution (50 mL) and extracted with DCM (2×80 mL). To the organic layer was added MeOH:TEA (1:1, 10 mL). The mixture was stirred at rt for 10 min and then evaporated. The mixture was washed with brine. The organic layer was separated, dried over Na₂SO₄, filtered and evaporated. The crude was purified by silica gel (100-200) using 70% EtOAc in PE to afford methyl 6-fluoro-7-(5-(hydroxymethyl)-1,3-dimethyl-1H-pyrazol-4-yl)-3-(3-methoxy-3-oxopropyl)-1-methyl-1H-indole-2-carboxylate (15-3, 3.87 g, 9.592 mmol, 99%) as an off white solid. ¹H NMR, crude (400 MHz, CDCl₃) δ 7.66 (dd, J=8.8, 5.6 Hz, 1H), 6.99 (t, J=8.8 Hz, 1H), 4.50 (dd, J=7.2, 5.6 Hz, 2H), 3.99 (s, 3H), 3.92 (s, 3H), 3.67 (s, 3H), 3.57 (s, 3H), 3.53 (t, J=8.0 Hz, 2H), 2.67 (t, J=4.8 Hz, 2H), 2.07 (s, 3H).

To a stirred solution of methyl 6-fluoro-7-(5-(hydroxymethyl)-1,3-dimethyl-1H-pyrazol-4-yl)-3-(3-methoxy-3-oxopropyl)-1-methyl-1H-indole-2-carboxylate (15-3, 1 g, 2.398 mmol) in DCM (12 mL) was added SOCl₂ (2.1 mL, 2.877 mmol) at 0° C., and the mixture was stirred at rt for 30 min. The solvent was evaporated, diluted with DCM (20 mL) and washed with a sat. NaHCO₃ solution (10 mL). The organic layer was separated, dried over Na₂SO₄, filtered and evaporated to afford semi pure methyl 7-(5-(chloromethyl)-1,3-dimethyl-1H-pyrazol-4-yl)-6-fluoro-3-(3-methoxy-3-oxopropyl)-1-methyl-1H-indole-2-carboxylate (15-4, 1 g, 22.98 mmol) as a brown sticky liquid that was used without further purification. MS (LCMS) m/z 436.38 [M+H]⁺.

To a stirred solution of semi pure methyl 7-(5-(chloromethyl)-1,3-dimethyl-1H-pyrazol-4-yl)-6-fluoro-3-(3-methoxy-3-oxopropyl)-1-methyl-1H-indole-2-carboxylate (15-4, 1 g, 2.29 mmol) in dry MeCN (16 mL) was added NaI (0.617 g, 4.123 mmol) at rt, and the mixture was heated to 80° C. for 2.5 h. After completion of the reaction, the solvent was evaporated. The mixture was diluted with water (10 mL) and extracted with EtOAc (2×15 mL). The organic layer was separated, dried over Na₂SO₄, filtered and evaporated to afford semi pure methyl 6-fluoro-7-(5-(iodomethyl)-1,3-dimethyl-1H-pyrazol-4-yl)-3-(3-methoxy-3-oxopropyl)-1-methyl-1H-indole-2-carboxylate (15-5, 1.2 g, 2.27 mmol) as a light brown sticky solid that was used without further purification. MS (LCMS) m/z 528.42 [M+H]⁺.

To a stirred solution of semi pure methyl 6-fluoro-7-(5-(iodomethyl)-1,3-dimethyl-1H-pyrazol-4-yl)-3-(3-methoxy-3-oxopropyl)-1-methyl-1H-indole-2-carboxylate (15-5, 1.2 g, 2.27 mmol) in MeOH (15 mL), was added K₂CO₃ (0.754 g, 5.4 mmol), and the solution was degassed with Ar for 10 min. In another round bottom flask, 3-(((3-((acetylthio)methyl)-1-ethyl-1H-pyrazol-5-yl)methyl)thio)naphthalen-1-yl acetate (8, 1.03 g, 2.5 mmol) in MeOH (5 mL) was degassed with Argon for 10 min, and this solution was added to previous mixture dropwise. The mixture was stirred at rt for 16 h. The solvent was evaporated. The mixture was diluted with water (10 mL) and extracted with EtOAc (2×20 mL). The organic layer was separated, dried over Na₂SO₄, filtered and evaporated. The crude was purified by silica gel (100-200) using 70% EtOAc in PE to afford methyl 7-(5-((((1-ethyl-5-(((4-hydroxynaphthalen-2-yl)thio)methyl)-1H-pyrazol-3-yl)methyl)thio)methyl)-1,3-dimethyl-1H-pyrazol-4-yl)-6-fluoro-3-(3-methoxy-3-oxopropyl)-1-methyl-1H-indole-2-carboxylate (15-6, 0.7 g, 0.95 mmol, 42% for three steps) as an off white foamy solid. MS (LCMS) m/z 730.63 [M+H]⁺.

To a suspension of methyl 7-(5-((((1-ethyl-5-(((4-hydroxynaphthalen-2-yl)thio)methyl)-1H-pyrazol-3-yl)methyl)thio)methyl)-1,3-dimethyl-1H-pyrazol-4-yl)-6-fluoro-3-(3-methoxy-3-oxopropyl)-1-methyl-1H-indole-2-carboxylate (15-6, 700 mg, 0.960 mmol) in dry THF (5 mL) was added BH₃.THF (1.0 M in THF, 5.3 mL, 5.44 mmol) dropwise at 0° C., and the mixture was stirred at rt for 3 h. The reaction was quenched with MeOH (5 mL) and 6N HCl (5 mL) at 0° C., and the mixture was stirred at rt for 20 min. The mixture was further diluted with water (10 mL) and extracted with 10% MeOH in DCM (2×30 mL). The organic layer was washed with sat. NaHCO₃ (2×20 mL), dried over Na₂SO₄, filtered and evaporated to give semi pure 15-7 that was purified by silica gel column (100-200) by using 0.1% MeOH in DCM to afford methyl 7-(5-((((1-ethyl-5-(((4-hydroxynaphthalen-2-yl)thio)methyl)-1H-pyrazol-3-yl)methyl)thio)methyl)-1,3-dimethyl-1H-pyrazol-4-yl)-6-fluoro-3-(3-hydroxypropyl)-1-methyl-1H-indole-2-carboxylate (15-7, 400 mg, 0.57 mmol, 59%) as an off white foamy solid.

To a stirred solution of TPP (448 mg, 1.71 mmol) in toluene (5 mL) was added a solution of di-tert-butyl diazene-1,2-dicarboxylate (15-7, 393 mg, 1.71 mmol), methyl 7-(5-((((1-ethyl-5-(((4-hydroxynaphthalen-2-yl)thio)methyl)-1H-pyrazol-3-yl)methyl)thio)methyl)-1,3-dimethyl-1H-pyrazol-4-yl)-6-fluoro-3-(3-hydroxypropyl)-1-methyl-1H-indole-2-carboxylate (13, 2×300 mg, 0.427 mmol) in THF (5 mL) at 90° C. The mixture was stirred at 90° C. for 6 h. The reaction was quenched with water (10 mL) and extracted with EtOAc (2×20 mL). The organic layer was dried over Na₂SO₄, filtered and concentrated to obtain the crude. The crude was purified by silica gel column (100-200) by using 80% EtOAc in PE to afford methyl (Z)-6¹-ethyl-1⁶-fluoro-1¹,2¹,2³-trimethyl-1¹H,2¹H,6¹H-10-oxa-4,8-dithia-1(7,3)-indola-2(4,5),6(3,5)-dipyrazola-9(3,1)-naphthalenacyclotridecaphane-12-carboxylate (15-8, 90 mg, 0.239 mmol, 16% for two batches) as an off white solid. MS (LCMS) m/z 684.56 [M+H]⁺.

Methyl (Z)-6¹-ethyl-1⁶-fluoro-1¹,2¹,2³-trimethyl-1¹H,2¹H,6¹H-10-oxa-4,8-dithia-1(7,3)-indola-2(4,5),6(3,5)-dipyrazola-9(3,1)-naphthalenacyclotridecaphane-12-carboxylate (15-8, 150 mg, LCMS 99%) was purified by SFC purification to afford 15-8-peak-1 (58 mg) as an off white solid and 15-8-peak-2 (54 mg) as an off white solid. These two peaks were separately used for the next steps to get respective final compound.

To a nitrogen degassed solution of methyl (Z)-1⁶-fluoro-6¹-isobutyl-1¹,2¹,2³-trimethyl-1¹H,2¹H,6¹H-10-oxa-4,8-dithia-1(7,3)-indola-2(4,5),6(3,5)-dipyrazola-9(3,1)-naphthalenacyclotridecaphane-1²-carboxylate (15-8-14-peak-1) (58 mg, 0.084 mmol) in MeOH:THF:H₂O (1:1:1. 2 mL) was added LiOH.H₂O (53 mg, 1.27 mmol) at rt, and the mixture was stirred at 60° C. for 1 h. The solvent was evaporated. The mixture was diluted with water (1 mL), acidified to pH 2 using 1N aq. HCl and filtered to afford ((Z)-6¹-ethyl-1⁶-fluoro-1¹,2¹,2³-trimethyl-1¹H,2¹H,6¹H-10-oxa-4,8-dithia-1(7,3)-indola-2(4,5),6(3,5)-dipyrazola-9(3,1)-naphthalenacyclotridecaphane-1²-carboxylic acid (15A, 45 mg, 0.067 mmol, 80%) as an off white solid. MS (LCMS) m/z 670.60 [M+H]⁺. ¹H NMR (400 MHz, DMSO-d₆) δ 13.3 (br s, 1H), 8.12 (d, J=7.6 Hz, 1H), 7.80-7.77 (m, 1H), 7.74 (d, J=1.6 Hz, 1H), 7.52-7.47 (m, 2H), 7.36 (s, 1H), 6.78 (t, J=9.2 Hz, 1H), 6.67 (s, 1H), 4.70 (s, 1H), 4.29-4.17 (q, J=16 Hz, 2H), 4.07-4.01 (m, 3H), 3.90-3.87 (m, 1H), 3.76 (s, 3H), 3.40-3.51 (m, 5H), 3.29 (d, J=14.4 Hz 1H), 3.18-3.14 (m, 2H), 3.0 (d, J=14 Hz 1H), 2.39-2.37 (m, 1H), 2.24 (bs, 1H), 1.89 (s, 3H) 1.29 (t, J=7.2 Hz 3H).

To a nitrogen degassed solution of methyl (Z)-61-ethyl-16-fluoro-11,21,23-trimethyl-11H,21H,61H-10-oxa-4,8-dithia-1(7,3)-indola-2(4,5),6(3,5)-dipyrazola-9(3,1)-naphthalenacyclotridecaphane-12-carboxylate (15-8-peak-2, 54 mg, 0.079 mmol) in MeOH:THF:H₂O (1:1:1, 2 mL) was added LiOH.H₂O (49.7 mg, 1.18 mmol) at rt, and the mixture was stirred at 60° C. for 1 h. The solvent was evaporated. The mixture was diluted with water (1 mL), acidified to pH 2 using 1N aq. HCl and filtered to afford (Z)-6¹-ethyl-1⁶-fluoro-1¹,2¹,2³-trimethyl-1¹H,2¹H,6¹H-10-oxa-4,8-dithia-1(7,3)-indola-2(4,5),6(3,5)-dipyrazola-9(3,1)-naphthalenacyclotridecaphane-12-carboxylic acid (15B, 48.6 mg, 0.073 mmol, 91%) as an off white solid. MS (LCMS) m/z 670.60 [M+H]⁺. ¹H NMR (400 MHz, DMSO-d₆) δ 13.2 (br s, 1H), 8.12 (d, J=7.2 Hz, 1H), 7.80-7.78 (m, 1H), 7.74 (d, J=1.6 Hz, 1H), 7.50-7.47 (m, 2H), 7.36 (s, 1H), 6.78 (t, J=9.2 Hz, 1H), 6.67 (s, 1H), 4.72 (s, 1H), 4.20 (m, 2H), 4.07-3.96 (m, 3H), 3.90-3.87 (m, 1H), 3.75 (3, 3H), 3.5-3.42 (m, 5H), 3.29 (d, J=14.4 Hz, 1H), 3.17-3.10 (m, 2H), 3.0 (d, J=7.2 Hz 1H), 2.40-2.37 (m, 1H), 2.24 (bs, 1H), 1.89 (s, 3H),1.28 (t, J=7.2 Hz, 3H).

The absolute stereochemistry of compounds (15A) and (15B) is arbitrarily assigned.

### Example 16

### (Z)-1⁶-Fluoro-6¹-isopropyl-1¹,2¹,2³-trimethyl-1¹H,2¹H,6¹H-10-oxa-4,8-dithia-1(7,3)-indola-2(4,5),6(3,5)-dipyrazola-9(3,1)-naphthalenacyclotridecaphane-1²-carboxylic acid

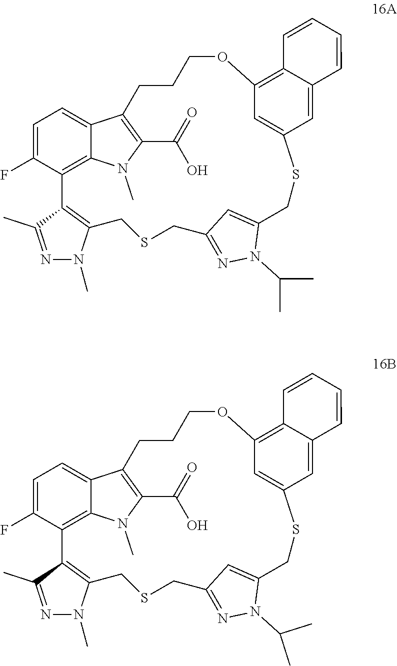

Following the same procedure of example 15, using intermediate 9 to obtain compounds 16A and 16 B.

16A: MS (LCMS) m/z 684.60 [M+H]⁺. ¹H NMR (400 MHz, DMSO-d₆) δ 13.2 (br s, 1H), 8.08 (d, J=7.6 Hz, 1H), 7.92 (dd, J=8.4, 5.2 Hz, 1H), 7.70 (d, J=7.6 Hz, 1H), 7.49-7.42 (m, 2H), 7.33 (s, 1H), 6.91 (t, J=9.2 Hz, 1H), 6.75 (s, 1H), 4.74 (s, 1H), 4.63-4.59 (m, 1H), 4.29 (s, 2H), 4.18-4.14 (m, 1H), 3.91-3.86 (m, 1H), 3.75 (s, 3H), 3.53 (s, 3H), 3.47-3.41 (m, 2H), 3.21-3.11 (m, 4H), 2.50-2.25 (m, 2H), 2.00 (s, 3H), 1.32 (q, J=6.4 Hz, 6H).

16B: MS (LCMS) m/z 684.60 [M+H]⁺. ¹H NMR (400 MHz, DMSO-d₆) δ 13.2 (br s, 1H), 8.08 (d, J=7.6 Hz, 1H), 7.92 (dd, J=8.4, 5.2 Hz, 1H), 7.70 (d, J=7.6 Hz, 1H), 7.49-7.42 (m, 2H), 7.33 (s, 1H), 6.91 (t, J=9.2 Hz, 1H), 6.75 (s, 1H), 4.74 (s, 1H), 4.63-4.59 (m, 1H), 4.29 (s, 2H), 4.18-4.14 (m, 1H), 3.91-3.86 (m, 1H), 3.75 (s, 3H), 3.53 (s, 3H), 3.47-3.41 (m, 2H), 3.21-3.11 (m, 4H), 2.50-2.25 (m, 2H), 2.00 (s, 3H), 1.32 (q, J=6.4 Hz 6H).

The absolute stereochemistry of compounds (16A) and (16B) is arbitrarily assigned.

### Example 17

### (Z)-1⁶-Fluoro-6¹-isobutyl-1¹,2¹,2³-trimethyl-1¹H,2¹H,6¹H-10-oxa-4,8-dithia-1(7,3)-indola-2(4,5),6(3,5)-dipyrazola-9(3,1)-naphthalenacyclotridecaphane-1²-carboxylic acid

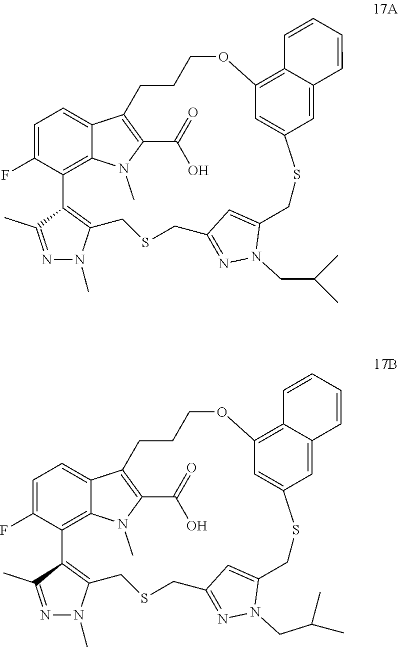

Following the same procedure of example 15, using intermediate 11 to obtain compounds 17A and 17 B.

17A: MS (LCMS) m/z 698.57 [M+H]⁺. ¹H NMR (400 MHz, DMSO-d₆) δ 13.2 (br s, 1H), 8.12 (d, J=7.6 Hz, 1H), 7.77-7.71 (m, 2H), 7.51-7.45 (m, 2H), 7.35 (m, 1H), 6.74 (t, J=9.2 Hz, 1H), 6.67 (s, 1H), 4.78 (s, 1H), 4.32-4.16 (m, 2H), 4.05-4.01 (m, 1H), 3.88-3.87 (m, 1H), 3.56 (brs, 5H), 3.50-3.45 (m, 5H), 3.19-3.02 (m, 4H), 2.36-2.08 (m, 3H), 1.88 (s, 3H), 0.80 (d, J=6.8 Hz, 6H).

17B: MS (LCMS) m/z 698.57 [M+H]⁺. ¹H NMR (400 MHz, DMSO-d₆) δ 13.2 (br s, 1H), 8.12 (d, J=7.6 Hz, 1H), 7.77-7.71 (m, 2H), 7.51-7.45 (m, 2H), 7.35 (m, 1H), 6.74 (t, J=9.2 Hz, 1H), 6.67 (s, 1H), 4.78 (s, 1H), 4.32-4.16 (m, 2H), 4.05-4.01 (m, 1H), 3.88-3.87 (m, 1H), 3.56 (brs, 5H), 3.50-3.45 (m, 5H), 3.19-3.02 (m, 4H), 2.36-2.08 (m, 3H), 1.88 (s, 3H), 0.80 (d, J=6.8 Hz, 6H).

The absolute stereochemistry of compounds (17A) and (17B) is arbitrarily assigned.

### Example 18

### (Z)-1⁶-Fluoro-6¹-isobutyl-1¹,2¹,2³-trimethyl-1¹H,2¹H,6¹H-10-oxa-4,8-dithia-1(7,3)-indola-2(4,5),6(3,5)-dipyrazola-9(3,1)-naphthalenacyclotridecaphane-1²-carboxylic acid

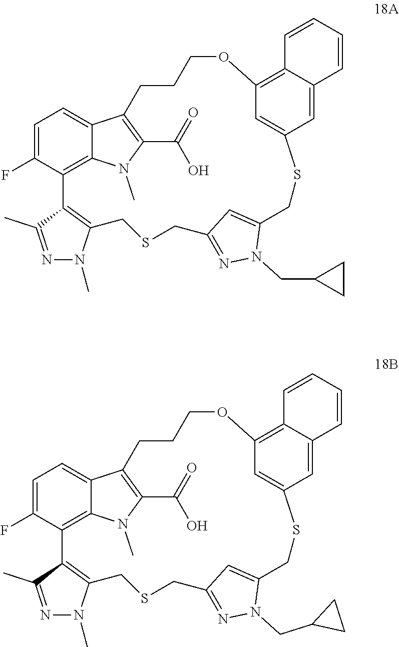

Following the same procedure of example 15, using intermediate 10 to obtain compounds 18A and 18 B.

18A: MS (ESI) m/z 696.40 [M+1]⁺. ¹H NMR (400 MHz, DMSO-d₆) δ 13.20 (br s, 1H), 8.12 (d, J=8.8 Hz, 1H), 7.80-7.70 (m, 2H), 7.55-7.45 (m, 2H), 7.36 (s, 1H), 6.75 (t, J=9.2 Hz, 1H), 6.67 (s, 1H), 4.77 (s, 1H), 4.30-4.15 (m, 2H), 4.05-4.00 (m, 1H), 3.95-3.80 (m, 3H), 3.75 (s, 3H), 3.55-3.40 (m, 5H), 3.20-3.10 (m, 3H), 3.03 (d, J=14.0 Hz, 1H), 2.40-2.30 (m, 1H), 2.30-2.20 (m, 1H), 1.90 (s, 3H), 1.30-1.20 (m, 1H), 0.50-0.45 (m, 2H), 0.35-0.30 (m, 2H); LCMS purity: 98.70%; HPLC purity: 98.14%; Chiral purity: 99.10%.

18B: MS (ESI) m/z 696.40 [M+1]⁺. ¹H NMR (400 MHz, DMSO-d₆) δ 13.20 (br s, 1H), 8.12 (d, J=8.8 Hz, 1H), 7.80-7.70 (m, 2H), 7.55-7.45 (m, 2H), 7.36 (s, 1H), 6.75 (t, J=9.2 Hz, 1H), 6.67 (s, 1H), 4.77 (s, 1H), 4.30-4.15 (m, 2H), 4.05-4.00 (m, 1H), 3.95-3.80 (m, 3H), 3.75 (s, 3H), 3.55-3.40 (m, 5H), 3.20-3.10 (m, 3H), 3.03 (d, J=14.0 Hz, 1H), 2.40-2.30 (m, 1H), 2.30-2.20 (m, 1H), 1.90 (s, 3H), 1.30-1.20 (m, 1H), 0.50-0.45 (m, 2H), 0.35-0.30 (m, 2H); LCMS purity: 99.07%; HPLC purity: 99.22%; Chiral purity: 99.95%.

The absolute stereochemistry of compounds (18A) and (18B) is arbitrarily assigned.

### Example 19

### (Z)-1⁶-Chloro-1¹,2¹,2³,6¹-tetramethyl-1¹H,2¹H,6¹H-10-oxa-4,8-dithia-1(7,3)-indola-2(4,5),6(3,5)-dipyrazola-9(3,1)-naphthalenacyclotridecaphane-1²-carboxylic acid

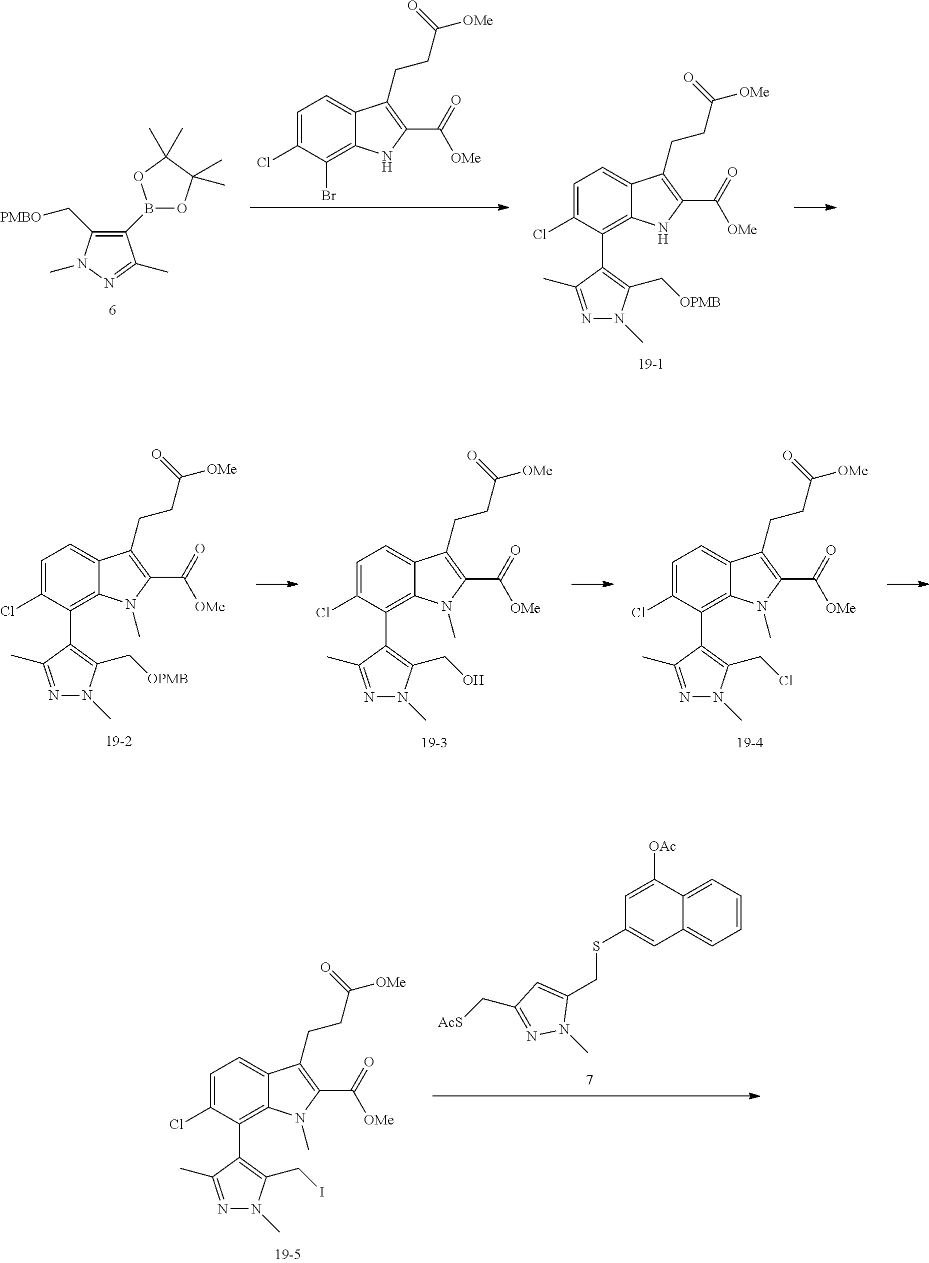

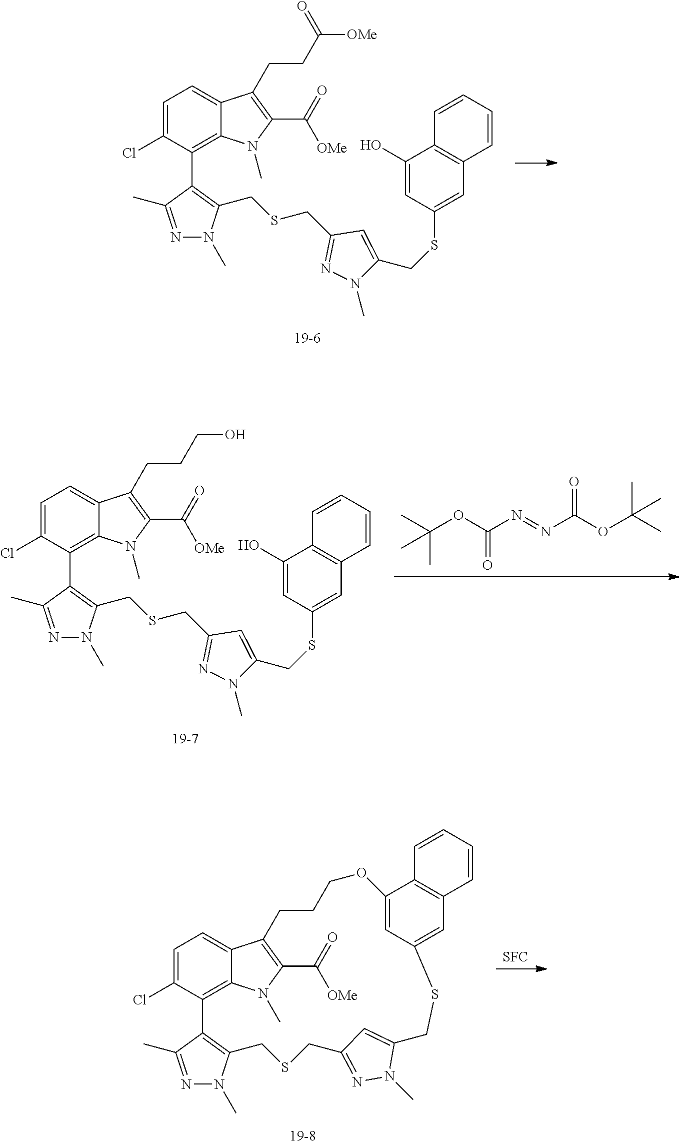

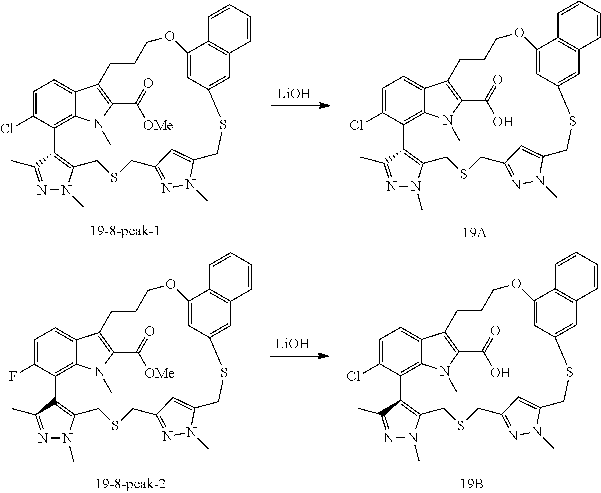

In a round bottom flask, to a argon degassed solution of methyl 7-bromo-6-chloro-3-(3-methoxy-3-oxopropyl)-1H-indole-2-carboxylate (from intermediate 2 synthesis) (5 g, 13.431 mmol) in dioxane (70 mL) were added 5-(((4-methoxybenzyl)oxy)methyl)-1,3-dimethyl-4-(4,4,5,5-tetramethyl-1,3,2-dioxaborolan-2-yl)-1H-pyrazole (6) (2×5 g, 13.431 mmol), Cs₂CO₃ (8.7 g, 26.862 mmol) and Pd(PPh3)₄ (1.55 g, 1.343 mmol). The mixture was heated at 100° C. for 16 h. The mixture was diluted with EtOAc (250 mL) and passed through a Celite pad that was washed with EtOAc (2×250 mL). The mixture was washed with water (3×150 mL), dried over Na₂SO₄, filtered and evaporated to afford semi pure 7 that was purified by silica gel (100-200) eluting with 70% EtOAc in PE to afford methyl 6-chloro-3-(3-methoxy-3-oxopropyl)-7-(5-(((4-methoxybenzyl)oxy)methyl)-1,3-dimethyl-1H-pyrazol-4-yl)-1H-indole-2-carboxylate (19-1, 8 g, 14.814 mmol, 58%) as a brown sticky liquid. MS (LCMS) m/z 540.76 [M+1]⁺.

To a stirred solution of methyl 6-chloro-3-(3-methoxy-3-oxopropyl)-7-(5-(((4-methoxybenzyl)oxy)methyl)-1,3-dimethyl-1H-pyrazol-4-yl)-1H-indole-2-carboxylate (19-1, 8 g, 14.814 mmol) in dry DMF (90 mL) were added Cs₂CO₃ (7.2 g, 22.221 mmol) and Mel (1.8 mL, 29.628 mmol) at 0° C., and the mixture was stirred at rt for 1 h. The reaction was quenched with water (300 mL) and extracted with EtOAc (2×300 mL). The organic layer was separated, dried over Na₂SO₄, filtered and evaporated to afford methyl 6-chloro-3-(3-methoxy-3-oxopropyl)-7-(5-(((4-methoxybenzyl)oxy)methyl)-1,3-dimethyl-1H-pyrazol-4-yl)-1-methyl-1H-indole-2-carboxylate (19-2, 8 g, 14.439 mmol,) as a brown viscous liquid. MS (LCMS) m/z 554.4 0 [M+1]⁺.

To a stirred solution of methyl 6-chloro-3-(3-methoxy-3-oxopropyl)-7-(5-(((4-methoxybenzyl)oxy)methyl)-1,3-dimethyl-1H-pyrazol-4-yl)-1-methyl-1H-indole-2-carboxylate (19-2, 8 g, 14.439 mmol) in DCM (80 mL) was added TFA (11.04 mL, 144.39 mmol) at 0° C., and the mixture was stirred at rt for 1 h. The solvent was evaporated. The reaction was quenched with a sat. aq. NaHCO₃ solution (200 mL) and extracted with DCM (3×400 mL). To the organic layer was added MeOH:TEA (1:1, 50 mL). The mixture was stirred at rt for 10 min and then evaporated. The mixture was washed with brine. The organic layer was separated, dried over Na₂SO₄, filtered and evaporated. The crude was purified by silica gel (100-200) using 10% MeOH in DCM to afford methyl 6-chloro-7-(5-(hydroxymethyl)-1,3-dimethyl-1H-pyrazol-4-yl)-3-(3-methoxy-3-oxopropyl)-1-methyl-1H-indole-2-carboxylate (19-3, 4 g, 9.218 mmol, 44% over three steps) as an off white solid. MS (LCMS) m/z 434.38 [M+H]⁺.

To a stirred solution of methyl 6-chloro-7-(5-(hydroxymethyl)-1,3-dimethyl-1H-pyrazol-4-yl)-3-(3-methoxy-3-oxopropyl)-1-methyl-1H-indole-2-carboxylate (19-3, 4 g, 9.218 mmol) in DCM (40 mL) was added SOCl₂ (0.8 mL, 11.062 mmol) at 0° C., and the mixture was stirred at rt for 30 min. The solvent was evaporated, diluted with DCM (200 mL) and washed with a sat. NaHCO₃ solution (50 mL). The organic layer was separated, dried over Na₂SO₄, filtered and evaporated to afford semi pure methyl 6-chloro-7-(5-(chloromethyl)-1,3-dimethyl-1H-pyrazol-4-yl)-3-(3-methoxy-3-oxopropyl)-1-methyl-1H-indole-2-carboxylate (19-4, 4 g, 8.843 mmol) as a brown sticky liquid that was used without further purification. MS (LCMS) m/z 452.32 [M+H]⁺.

To a stirred solution of semi pure methyl 6-chloro-7-(5-(chloromethyl)-1,3-dimethyl-1H-pyrazol-4-yl)-3-(3-methoxy-3-oxopropyl)-1-methyl-1H-indole-2-carboxylate (19-4, 4 g, 8.843 mmol) in dry MeCN (40 mL) was added NaI (2.37 g, 15.917 mmol) at rt, and the mixture was heated to 80° C. for 2.5 h. The solvent was evaporated. The mixture was diluted with water (150 mL) and extracted with EtOAc (2×2500 mL). The organic layer was separated, dried over Na₂SO₄, filtered and evaporated to afford semi pure methyl 6-chloro-7-(5-(iodomethyl)-1,3-dimethyl-1H-pyrazol-4-yl)-3-(3-methoxy-3-oxopropyl)-1-methyl-1H-indole-2-carboxylate (19-5, 5 g, 9.194 mmol) as a light brown sticky solid that was used without further purification. MS (LCMS) m/z 544.31 [M+H]⁺.

To a stirred solution of semi pure methyl 6-chloro-7-(5-(iodomethyl)-1,3-dimethyl-1H-pyrazol-4-yl)-3-(3-methoxy-3-oxopropyl)-1-methyl-1H-indole-2-carboxylate (19-5, 4.4 g, 8.091 mmol) in MeOH (40 mL), THF (5 mL) and K₂CO₃ (2.679 g, 19.418 mmol) were added. The solution was degassed with Ar for 10 min. In another round bottom flask, 3-(((3-((acetylthio)methyl)-1-methyl-1H-pyrazol-5-yl)methyl)thio)naphthalen-1-yl acetate (7, 3.560 g, 8.900 mmol) in MeOH (30 mL) and THF (5 mL) was degassed with Ar for 10 min, and this solution was added to previous mixture dropwise. The solution was stirred at rt for 16 h. The solvent was evaporated. The mixture was diluted with water (100 mL) and extracted with EtOAc (2×250 mL). The organic layer was separated, dried over Na₂SO₄, filtered and evaporated. The crude was purified by silica gel (100-200) using 80% EtOAc in PE to afford methyl 6-chloro-7-(5-((((5-(((4-hydroxynaphthalen-2-yl)thio)methyl)-1-methyl-1H-pyrazol-3-yl)methyl)thio)methyl)-1,3-dimethyl-1H-pyrazol-4-yl)-3-(3-methoxy-3-oxopropyl)-1-methyl-1H-indole-2-carboxylate (19-6, 2.4 g, 3.277 mmol, 41% for three steps) as an off white foamy solid. MS (LCMS) m/z 732.54 [M+H]⁺.

To a suspension of methyl 6-chloro-7-(5-((((5-(((4-hydroxynaphthalen-2-yl)thio)methyl)-1-methyl-1H-pyrazol-3-yl)methyl)thio)methyl)-1,3-dimethyl-1H-pyrazol-4-yl)-3-(3-methoxy-3-oxopropyl)-1-methyl-1H-indole-2-carboxylate (19-6, 2×1.2 g, 1.638 mmol) in dry THF (30 mL) was added BH₃.THF (1.0 M in THF, 9 mL, 9.012 mmol) dropwise at 0° C., and the mixture was stirred at rt for 16 h. The reaction was quenched with MeOH (15 mL) and 6N HCl (15 mL) at 0° C., and the mixture was stirred at rt for 20 min. The mixture was further diluted with water (50 mL) and extracted with 10% MeOH in DCM (2×150 mL). The organic layer was separated and evaporated. The crude was dissolved in 10% MeOH in DCM (200 mL), washed with sat. NaHCO₃ (2×100 mL), dried over Na₂SO₄, filtered and evaporated to give the semi pure compound that was purified by silica gel column (100-200) purification using 100% EtOAc to afford methyl 6-chloro-7-(5-((((5-(((4-hydroxynaphthalen-2-yl)thio)methyl)-1-methyl-1H-pyrazol-3-yl)methyl)thio)methyl)-1,3-dimethyl-1H-pyrazol-4-yl)-3-(3-hydroxypropyl)-1-methyl-1H-indole-2-carboxylate (19-7, 1.1 g, 1.561 mmol, 47%) as an off white foamy solid. MS (LCMS) m/z 704.48 [M+H]⁺. ¹H NMR (400 MHz, CDCl₃) δ 9.17 (bs, 1H), 8.25 (d, 1H), 7.69 (dd, 1H), 7.58 (dd, 1H), 7.45-7.55 (m, 2H), 7.39 (s, 1H), 7.15 (d, 1H), 6.75 (s, 1H), 5.31 (s, 1H), 3.85-4.00 (m, 8H), 3.8-3.7 (m, 2H), 3.65 (s, 3H), 3.4-3.5 (m, 6H), 3.19 (t, 2H), 2.10 (s, 3H), 1.95 (m, 2H), 1.2 (t, 1H).

To a stirred solution of TPP (371 mg, 1.419 mmol) in toluene (14 mL) was heated to 90° C. then added a solution of di-tert-butyl diazene-1,2-dicarboxylate (371 mg, 1.419 mmol), methyl 6-chloro-7-(5-((((5-(((4-hydroxynaphthalen-2-yl)thio)methyl)-1-methyl-1H-pyrazol-3-yl)methyl)thio)methyl)-1,3-dimethyl-1H-pyrazol-4-yl)-3-(3-hydroxypropyl)-1-methyl-1H-indole-2-carboxylate (19-7, 2×500 mg, 0.709 mmol) in THF (14 mL). The mixture was stirred at 90° C. for 2 h. The reaction was quenched with water (50 mL) and extracted with EtOAc (2×200 mL). The organic layer was dried over Na₂SO₄, filtered, concentrated and was purified by silica gel column (100-200) purification using 100% EtOAc to afford methyl (Z)-1⁶-chloro-1¹,2¹,2³,6¹-tetramethyl-1¹H,2¹H,6¹H-10-oxa-4,8-dithia-1(7,3)-indola-2(4,5),6(3,5)-dipyrazola-9(3,1)-naphthalenacyclotridecaphane-1²-carboxylate (19-8, (275 mg, 0.400 mmol, 28%) as an off white solid. MS (LCMS) m/z 686.08 [M+H]⁺.

Methyl (Z)-1⁶-chloro-1¹,2¹,2³,6¹-tetramethyl-1¹H,2¹H,6¹H-10-oxa-4,8-dithia-1(7,3)-indola-2(4,5),6(3,5)-dipyrazola-9(3,1)-naphthalenacyclotridecaphane-1²-carboxylate (19-8, 275 mg) was purified by SFC purification to afford 19-8-peak-1 (90 mg) as an off white solid with 99.96% of LCMS purity (chiral HPLC:99.19%) and 19-8-peak-2 (90 mg) as an off white solid with 98.54% of LCMS purity (chiral HPLC:99.69%). These two peaks were separately used for the next steps to get respective final compound.

19-8-peak-1: MS (LCMS) m/z 686.47 [M+H]⁺.

19-8-peak-2: MS (LCMS) m/z 686.83 [M+H]⁺.

To a nitrogen degassed solution of methyl (Z)-16-chloro-11,21,23,61-tetramethyl-11H,21H,61H-10-oxa-4,8-dithia-1(7,3)-indola-2(4,5),6(3,5)-dipyrazola-9(3,1)-naphthalenacyclotridecaphane-12-carboxylate (19-8-peak-1, 90 mg, 0.131 mmol) in MeOH:THF:H₂O (1:1:1, 1 mL) was added LiOH.H₂O (82 mg, 1.967 mmol) at rt, and the mixture was stirred at 80° C. for 3 h. The solvent was evaporated. The mixture was diluted with water (1 mL), acidified to pH 2 using 1N aq. HCl and filtered to afford (Z)-1⁶-chloro-1¹,2¹,2³,6¹-tetramethyl-1¹H,2¹H,6¹H-10-oxa-4,8-dithia-1(7,3)-indola-2(4,5),6(3,5)-dipyrazola-9(3,1)-naphthalenacyclotridecaphane-1²-carboxylic acid (19A, 65 mg, 0.096 mmol, 73%) as an off white solid. MS (LCMS) m/z 672.47 [M+H]⁺. ¹H NMR (400 MHz, DMSO-d₆) δ 13.2 (br s, 1H), 8.13 (d, 1H), 7.75 (d, 2H), 7.50 (m, 2H), 7.40 (s, 1H), 7.0 (d, 1H), 6.58 (s, 1H), 4.72 (s, 1H), 4.15-4.30 (m, 2H), 3.95 (m, 1H), 3.85 (m, 1H), 3.65 (s, 3H), 3.55 (s, 3H), 3.40-3.51 (m, 5H), 3.20-3.30 (m, 1H), 3.1-3.20 (m, 2H), 2.90 (d, 1H), 2.30-2.40 (m, 1H), 2.15-2.25 (m, 1H), 1.85 (s, 3H).

To a nitrogen degassed solution of methyl (Z)-1⁶-chloro-1¹,2¹,2³,6¹-tetramethyl-1¹H,2¹H,6¹H-10-oxa-4,8-dithia-1(7,3)-indola-2(4,5),6(3,5)-dipyrazola-9(3,1)-naphthalenacyclotridecaphane-1²-carboxylate (19-8-peak-2, 90 mg, 0.131 mmol) in MeOH:THF:H₂O (1:1:1, 1 mL) was added LiOH.H₂O (82 mg, 1.967 mmol) at rt, and the mixture was stirred at 80° C. for 3 h. The solvent was evaporated. The mixture was diluted with water (1 mL), acidified to pH 2 using 1N aq. HCl and filtered to afford (Z)-1⁶-chloro-1¹,2¹,2³,6¹-tetramethyl-1¹H,2¹H,6¹H-10-oxa-4,8-dithia-1(7,3)-indola-2(4,5),6(3,5)-dipyrazola-9(3,1)-naphthalenacyclotridecaphane-1²-carboxylic acid (19B, 63 mg, 0.093 mmol, 71%) as an off white solid. MS (LCMS) m/z 672.47 [M+H]⁺. ¹H NMR (400 MHz, DMSO-d₆) δ 13.3 (br s, 1H), 8.13 (d, 1H), 7.75 (d, 2H), 7.50 (m, 2H), 7.40 (s, 1H), 7.0 (d, 1H), 6.58 (s, 1H), 4.72 (s, 1H), 4.15-4.30 (m, 2H), 3.95 (m, 1H), 3.85 (m, 1H), 3.65 (s, 3H), 3.55 (s, 3H), 3.40-3.51 (m, 5H), 3.20-3.30 (m, 1H), 3.1-3.20 (m, 2H), 2.90 (d, 1H), 2.30-2.40 (m, 1H), 2.15-2.25 (m, 1H), 1.87 (s, 3H).

The absolute stereochemistry of compounds (19A) and (19B) is arbitrarily assigned.

### Example 20

### (Z)-2¹-(Bicyclo[1.1.1]pentan-1-yl)-1⁶-chloro-1¹,2³,6¹-trimethyl-1¹H,2¹H,6¹H-10-oxa-4,8-dithia-1(7,3)-indola-2(4,5),6(3,5)-dipyrazola-9(3,1)-naphthalenacyclotridecaphane-1²-carboxylic acid

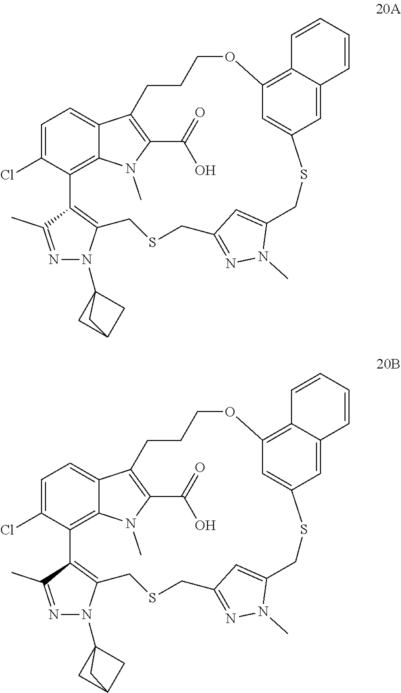

Following the same procedure of example 19, using intermediate 13 to obtain compounds 20A and 20 B.

20A: MS (LCMS) m/z 724.28 [M+H]⁺. ¹H NMR (400 MHz, DMSO-d₆) δ 8.16-8.12 (m, 1H), 7.78-7.72 (m, 2H), 7.54-7.45 (m, 3H), 7.05 (d, J=8.8 Hz, 1H), 6.49 (s, 1H), 4.75 (s, 1H), 4.30-4.10 (m, 2H), 3.95-3.86 (m, 1H), 3.85-3.80 (m, 1H), 3.66 (s, 3H), 3.58 (d, J=14.8 Hz, 1H), 3.51 (s, 3H), 3.45-3.40 (m, 3H), 3.22-3.17 (m, 1H), 3.15-3.02 (m, 2H), 2.80 (d, J=14.0 Hz, 1H), 2.60 (s, 1H), 2.40-2.30 (m, 6H), 2.38-2.25 (m 2H), 1.88 (s, 3H); LCMS purity: 98.12%; HPLC purity: 96.79%; Chiral: 98.56%.

20B: MS (LCMS) m/z 724.28 [M+H]⁺. ¹H NMR (400 MHz, DMSO-d₆) δ 8.16-8.12 (m, 1H), 7.78-7.72 (m, 2H), 7.54-7.45 (m, 3H), 7.05 (d, J=9.2 Hz, 1H), 6.48 (s, 1H), 4.74 (s, 1H), 4.28-4.10 (m, 2H), 3.95-3.86 (m, 1H), 3.85-3.75 (m, 1H), 3.66 (s, 3H), 3.58 (d, J=14.0 Hz, 1H), 3.51 (s, 3H), 3.45-3.40 (m, 3H), 3.22-3.17 (m, 1H), 3.15-3.02 (m, 2H), 2.80 (d, J=14.0 Hz, 1H), 2.59 (s, 1H), 2.40-2.30 (m, 6H), 2.38-2.25 (m 2H), 1.88 (s, 3H); LCMS purity: 99.44%; HPLC purity: 97.94%; Chiral: 96.19%.

The absolute stereochemistry of compounds (20A) and (20B) is arbitrarily assigned.

### Example 21

### (Z)-2¹-(Bicyclo[1.1.1]pentan-1-yl)-1⁶-chloro-1¹,2³-dimethyl-1¹H,2¹H,6¹H-10-oxa-4,8-dithia-1(7,3)-indola-2(4,5),6(3,5)-dipyrazola-9(3,1)-naphthalenacyclotridecaphane-1²-carboxylic acid

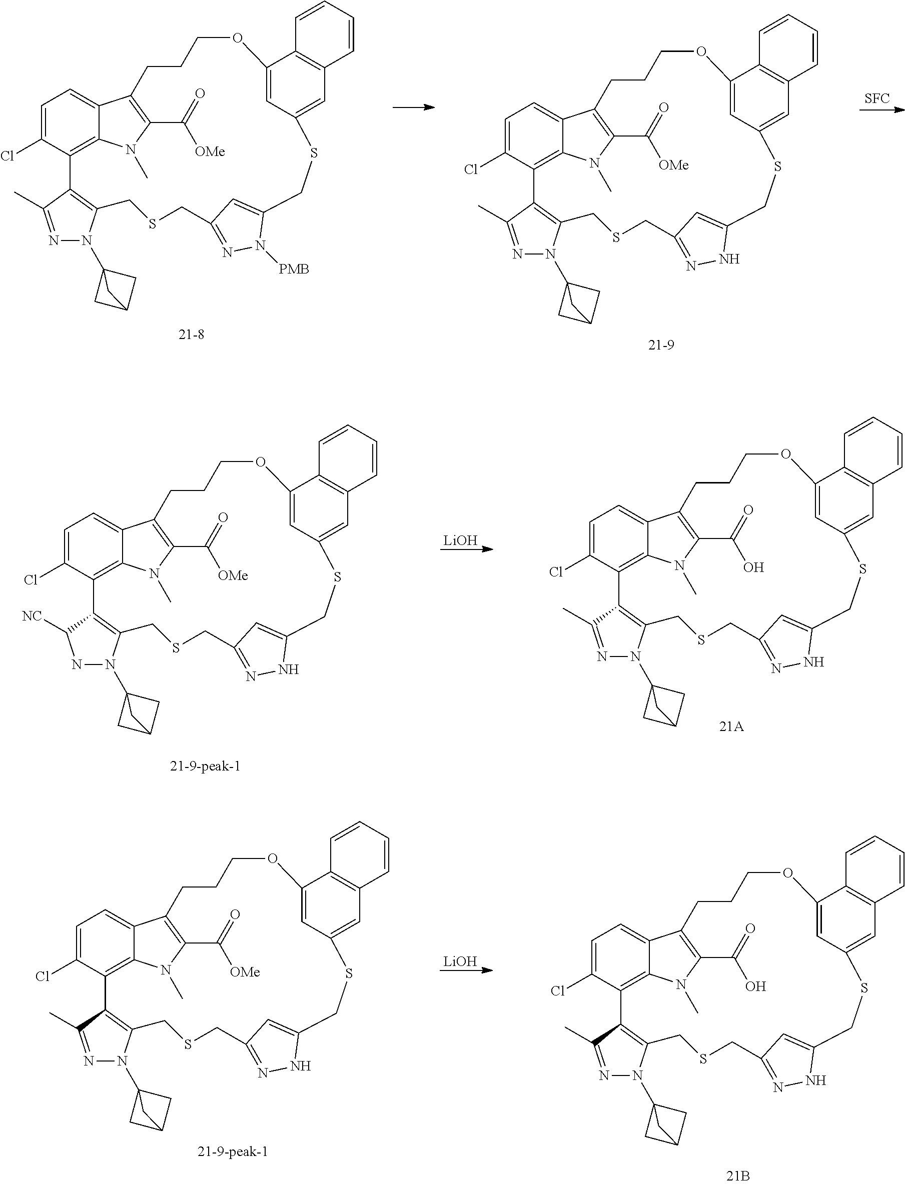

Following the same procedure of example 19, using corresponding intermediates 13 and 12 to obtain 21-8 as an off white solid. MS (ESI) m/z 844.53 [M+1]⁺.

In a pressure tube, to a stirred solution of methyl (Z)-2¹-(bicyclo[1.1.1]pentan-1-yl)-1⁶-chloro-6¹-(4-methoxybenzyl)-1¹,2³-dimethyl-1¹H,2¹H,6¹H-10-oxa-4,8-dithia-1(7,3)-indola-2(4,5),6(3,5)-dipyrazola-9(3,1)-naphthalenacyclotridecaphane-1²-carboxylate (21-8, 680 mg, 0.806 mmol) in TFA (16 mL) was added anisole (522 mg, 4.836 mmol) at rt, and the mixture was stirred at 90° C. for 16 h. After completion of reaction, the reaction was concentrated under reduced pressure. The crude was dissolved in CH₂Cl₂ (50 mL) washed with aq NaHCO₃ solution (50 mL), water (50 mL) and brine (50 mL). The separated organic layer was dried over anhydrous Na₂SO₄, filtered and concentrated under reduced pressure. The crude was purified by silica gel column and eluted with 60% EtOAc in PE to afford methyl (Z)-2¹-(bicyclo[1.1.1]pentan-1-yl)-1⁶-chloro-1¹,2³-dimethyl-1¹H,2¹H,6¹H-10-oxa-4,8-dithia-1(7,3)-indola-2(4,5),6(3,5)-dipyrazola-9(3,1)-naphthalenacyclotridecaphane-1²-carboxylate (21-9, 350 mg, 0.484 mmol, 45% over 2 steps) as an off-white solid. MS (ESI) m/z 724.40 [M+1]⁺.

Racemic methyl (Z)-2¹-(bicyclo[1.1.1]pentan-1-yl)-1⁶-chloro-1¹,2³-dimethyl-1¹H,2¹H,6¹H-10-oxa-4,8-dithia-1(7,3)-indola-2(4,5),6(3,5)-dipyrazola-9(3,1)-naphthalenacyclotridecaphane-1²-carboxylate (21-9, 350 mg) was purified by SFC purification to afford 21-9-peak-1 (130 mg) as an off white solid with 98% of LCMS purity (chiral HPLC: 99.60%) and 21-9-peak-2 (140 mg) as an off white solid with 95% of LCMS purity (chiral HPLC: 99.42%). These two peaks were separately used for the next steps to get respective final compound.

21-9-peak-1: MS (ESI) m/z 724.54 [M+1]⁺. ¹H NMR (400 MHz, CDCl₃) δ 8.31 (d, J=9.6 Hz, 1H), 7.72 (d, J=9.2 Hz, 1H), 7.55-7.48 (m, 5H), 7.03 (d, J=8.8 Hz, 1H), 6.27 (s, 1H), 3.96-3.92 (m, 2H), 3.80 (s, 3H), 3.69-3.49 (m, 9H), 3.23-3.13 (m, 3H), 2.90-2.80 (m, 1H), 2.61 (s, 1H), 2.41 (s, 6H), 2.30-2.20 (m, 1H), 2.05 (s 3H).

21-9-peak-2: MS (ESI) m/z 724.49 [M+1]⁺. ¹H NMR (400 MHz, CDCl₃) δ 8.31 (d, J=9.6 Hz, 1H), 7.72 (d, J=9.2 Hz, 1H), 7.55-7.48 (m, 5H), 7.03 (d, J=8.8 Hz, 1H), 6.27 (s, 1H), 3.96-3.92 (m, 2H), 3.80 (s, 3H), 3.69-3.49 (m, 9H), 3.23-3.13 (m, 3H), 2.90-2.80 (m, 1H), 2.61 (s, 1H), 2.41 (s, 6H), 2.30-2.20 (m, 1H), 2.05 (s 3H).

To a stirred solution of methyl (Z)-2¹-(bicyclo[1.1.1]pentan-1-yl)-1⁶-chloro-1¹,2³-dimethyl-1¹H,2¹H,6¹H-10-oxa-4,8-dithia-1(7,3)-indola-2(4,5),6(3,5)-dipyrazola-9(3,1)-naphthalenacyclotridecaphane-1²-carboxylate (21-9-peak-1, 130 mg, 0.179 mmol) in degassed MeOH:THF:H₂O (1:1:1, 6 mL) was added LiOH.H₂O (112 mg, 2.685 mmol) at rt, and the mixture was heated at 60° C. for 1 h. After completion of reaction, the mixture was concentrated under reduced pressure. The residue was acidified to pH-3 using 2 N aq. HCl. The solid was filtered and washed with water and pentane. The obtained compound was dissolved in MeCN:H₂O (1:1, 4 mL) and lyophilized to afford (Z)-2¹-(bicyclo[1.1.1]pentan-1-yl)-1⁶-chloro-1¹,2³-dimethyl-1¹H,2¹H,6¹H-10-oxa-4,8-dithia-1(7,3)-indola-2(4,5),6(3,5)-dipyrazola-9(3,1)-naphthalenacyclotridecaphane-1²-carboxylic acid (21A, 110 mg, 0.141 mmol, 86%) as an off-white solid. MS (ESI) m/z 710.53 [M+1]⁺. ¹H NMR (400 MHz, DMSO-d₆) δ 12.90 (br s, 1H), 8.15 (d, J=7.6 Hz, 1H), 7.75 (d, J=7.6 Hz, 1H), 7.68 (d, J=7.6 Hz, 1H), 7.52-7.45 (m, 2H), 7.40 (br s, 1H), 6.98 (d, J=8.4 Hz, 1H), 6.57 (s, 1H), 4.68 (s, 1H), 4.09-3.81 (m, 4H), 3.63-3.60 (m, 1H), 3.49 (s, 3H), 3.42-3.10 (m, 4H), 2.90-2.87 (m, 1H), 2.67 (s, 1H), 2.35-2.30 (m, 7H), 2.19-2.18 (m, 1H), 1.87 (s 3H).

To a stirred solution of methyl (Z)-2¹-(bicyclo[1.1.1]pentan-1-yl)-1⁶-chloro-1¹,2³-dimethyl-1¹H,2¹H,6¹H-10-oxa-4,8-dithia-1(7,3)-indola-2(4,5),6(3,5)-dipyrazola-9(3,1)-naphthalenacyclotridecaphane-1²-carboxylate (21-9-peak-2, 140 mg, 0.193 mmol) in degassed MeOH:THF:H₂O (1:1:1, 6 mL) was added LiOH.H₂O (121 mg, 2.895 mmol) at rt, and the mixture was heated at 60° C. for 1 h. After completion of reaction, the mixture was concentrated under reduced pressure. The residue was acidified to pH-3 using 2 N aq. HCl. The solid was filtered and washed with water and pentane. The obtained compound was dissolved in MeCN:H₂O (1:1, 4 mL) and lyophilized to afford (Z)-2¹-(bicyclo[1.1.1]pentan-1-yl)-1⁶-chloro-1¹,2³-dimethyl-1¹H,2¹H,6¹H-10-oxa-4,8-dithia-1(7,3)-indola-2(4,5),6(3,5)-dipyrazola-9(3,1)-naphthalenacyclotridecaphane-1²-carboxylic acid (21B, 110 mg, 0.141 mmol, 86%) as an off-white solid. MS (ESI) m/z 710.41 [M+1]⁺. ¹H NMR (400 MHz, DMSO-d₆) δ 12.90 (br s, 1H), 8.15 (d, J=8.0 Hz, 1H), 7.75 (d, J=8.0 Hz, 1H), 7.67 (d, J=8.0 Hz, 1H), 7.52-7.45 (m, 2H), 7.40 (br s, 1H), 6.98 (d, J=8.0 Hz, 1H), 6.56 (s, 1H), 4.70 (s, 1H), 4.10-3.81 (m, 4H), 3.64-3.60 (m, 1H), 3.49 (s, 3H), 3.42-3.12 (m, 4H), 2.91-2.87 (m, 1H), 2.69 (s, 1H), 2.49-2.33 (m, 7H), 2.19-2.18 (m, 1H), 1.87 (s 3H).

The absolute stereochemistry of compounds (21A) and (21B) is arbitrarily assigned.

### Example 22

### (Z)-2⁵-(tert-Butoxycarbonyl)-1⁶-chloro-1¹,6¹-dimethyl-2⁴,2⁵,2⁶,2⁷-tetrahydro-1¹H,6¹H-10-oxa-4,8-dithia-2(3,2)-pyrazolo[1,5-a]pyrazina-1(7,3)-indola-6(3,5)-pyrazola-9(3,1)-naphthalenacyclotridecaphane-1²-carboxylic acid

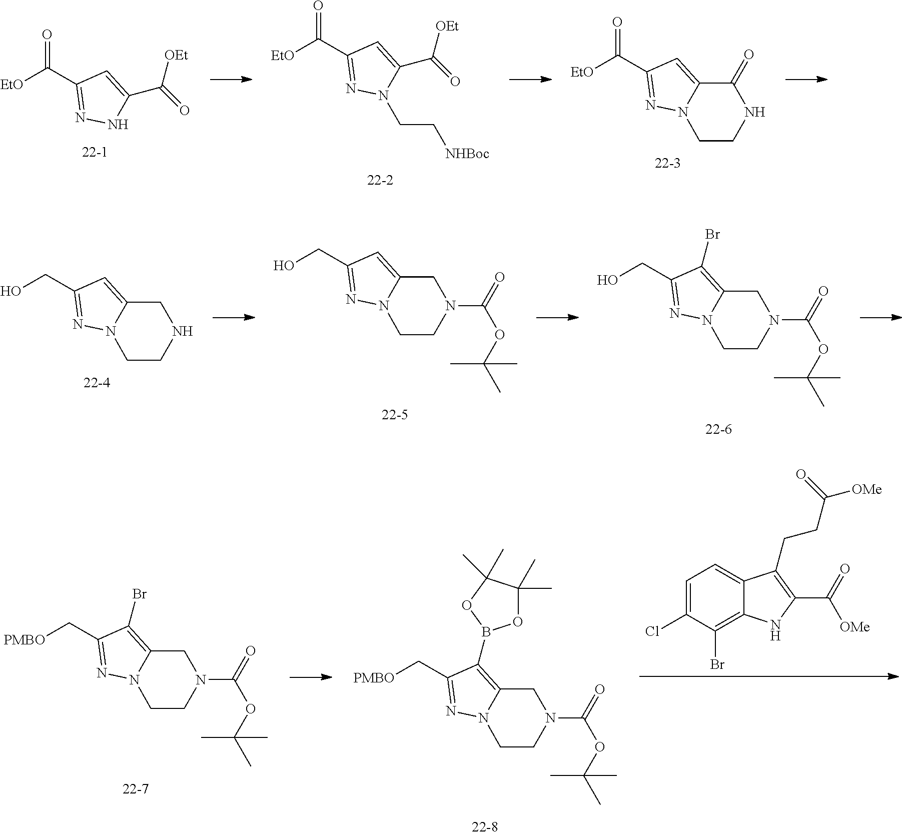

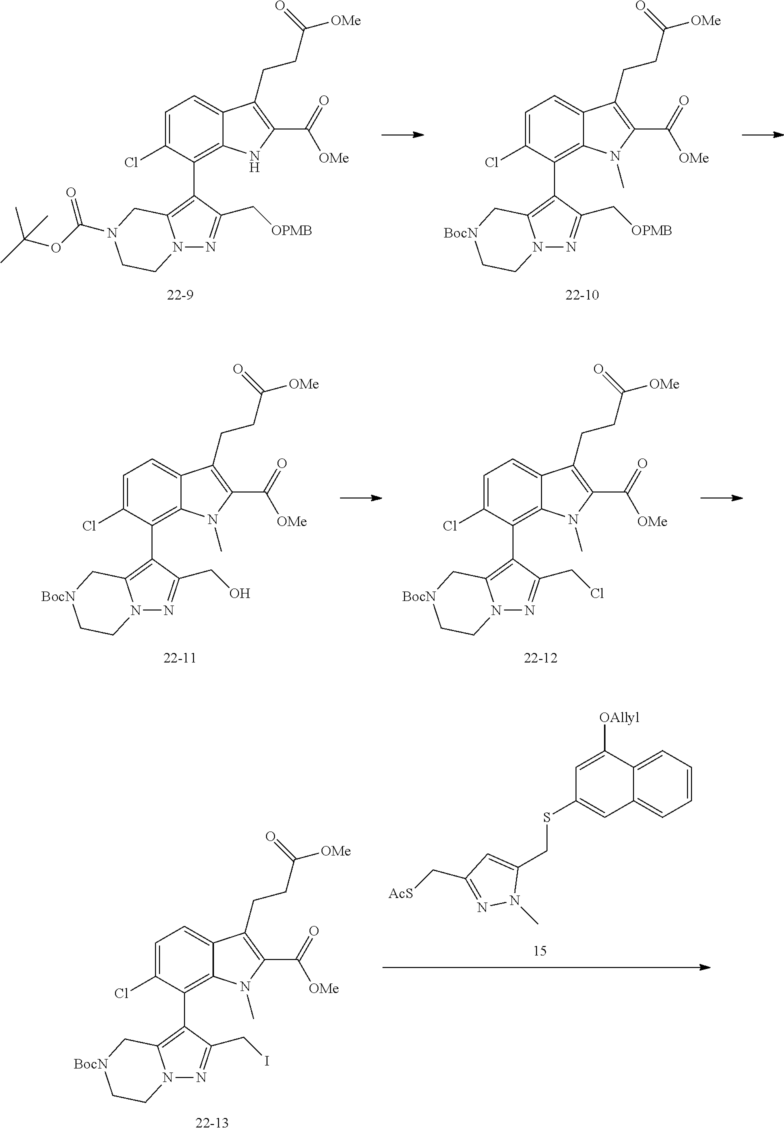

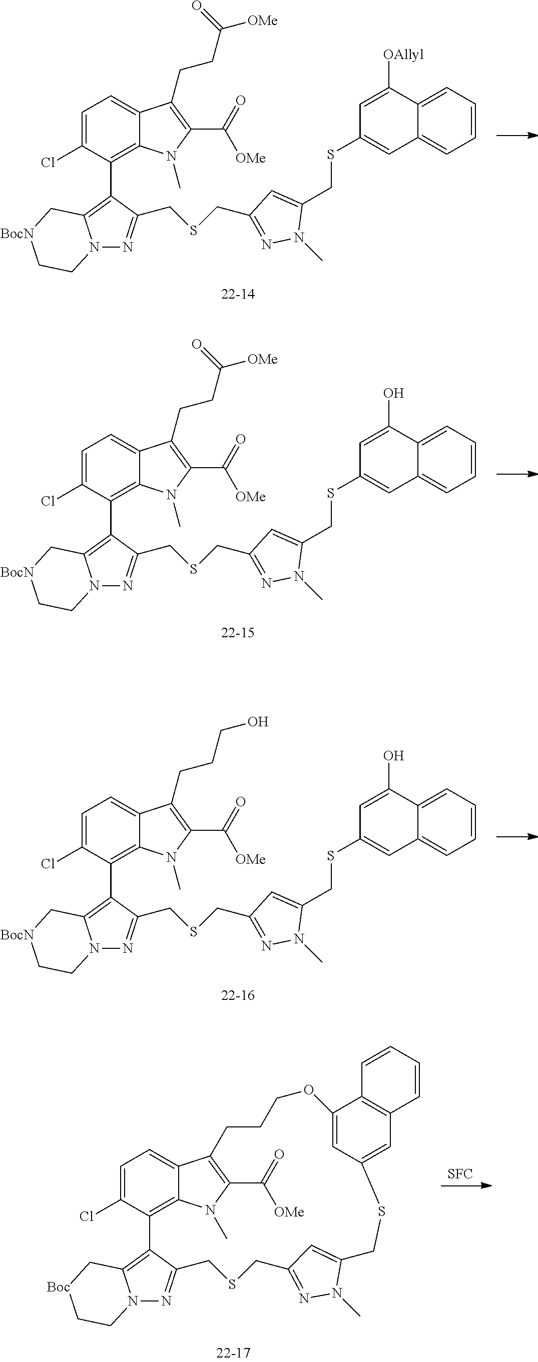

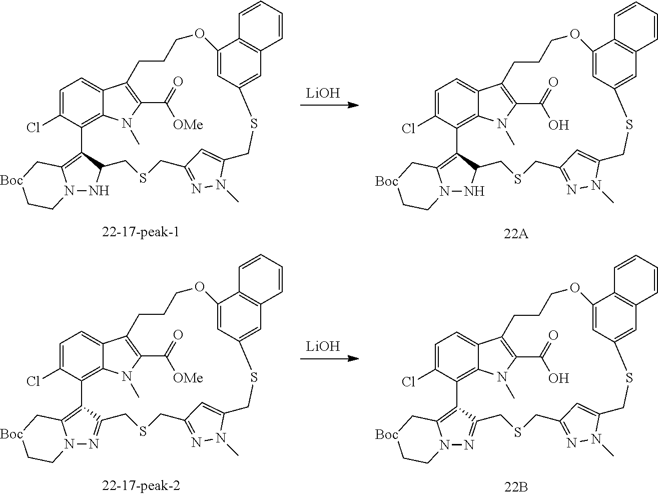

To a stirred solution of diethyl 1H-pyrazole-3,5-dicarboxylate (22-1, 25 g, 117.9 mmol) in DMF (1 L) were added Cs₂CO₃ (46.1 g, 141.5 mmol) and tert-butyl (2-bromoethyl)carbamate (32.87 g, 147.4 mmol) at 0° C., and the mixture was stirred at rt for 16 h. The mixture was diluted with water (2 L). The obtained white solid was filtered, washed with water (2×1 L), dried under high vacuum and triturated pentane to afford diethyl 1-(2-((tert-butoxycarbonyl)amino)ethyl)-1H-pyrazole-3,5-dicarboxylate (22-2, 38 g, 106.99 mmol, 91%) as a white solid that was used without further purification. MS (LCMS) m/z 356.14 [M+H]⁺. ¹H NMR (400 MHz, CDCl₃) δ 7.35 (s, 1H), 4.82 (br s, 1H), 4.76 (t, J=5.6 Hz, 2H), 4.44-4.33 (m, 4H), 2.59-2.68 (m, 2H), 1.42-1.37 (m, 15H).

Diethyl 1-(2-((tert-butoxycarbonyl)amino)ethyl)-1H-pyrazole-3,5-dicarboxylate (22-2, 28 g, 78.84 mmol) was added to 4N HCl in dioxane (280 mL) at 0° C., and the mixture was stirred at rt for 4 h. The mixture was evaporated and diluted with aq. Sat. NaHCO₃ (300 mL). The mixture was stirred at rt for 1 h and then extracted with DCM (3×500 mL). The organic layer was separated, dried over Na₂SO₄, filtered and evaporated to afford 3 that was further triturated with pentane and dried to give ethyl 4-oxo-4,5,6,7-tetrahydropyrazolo[1,5-a]pyrazine-2-carboxylate (22-3, 13.8 g, 66.0 mmol, 83%) as a white solid that was used without further purification. MS (LCMS) m/z 210.04 [M+H]⁺. ¹H NMR (400 MHz, CDCl₃) δ 7.38 (s, 1H), 6.69 (br s, 1H), 4.50 (t, J=1.2 Hz, 2H), 4.43 (q, J=7.2 Hz, 2H), 3.85-3.81 (m, 2H), 1.41 (t, J=6.8 Hz, 3H).

To a stirred solution of ethyl 4-oxo-4,5,6,7-tetrahydropyrazolo[1,5-a]pyrazine-2-carboxylate (22-3, 40.0 g, 191.31 mmol) in THF (800 mL) was added LiAlH₄ (2.4 M in THF, 319 mL, 765.6 mmol) at 0° C., and the mixture was stirred at 70° C. for 6 h. The mixture was cooled to 0° C. and diluted with EtOAc (1 L). Water (29.2 mL) was added dropwise, followed by 15% aq. NaOH solution (29.2 mL) and water (87.6 mL). The mixture was warmed to rt and then stirred for 15 min. Anhydrous sodium sulphate was added, and the mixture was stirred for 15 min. The mixture was filtered through Celite that washed several times with EtOAc. The mixture was filtered. The filtrate was dried over Na₂SO₄, filtered and evaporated to afford (4,5,6,7-tetrahydropyrazolo[1,5-a]pyrazin-2-yl)methanol (22-4, 52.0 g, 339.67 mmol, 83%, combined 40 g+45 g) as a clear viscous liquid that was used without further purification. MS (LCMS) m/z 154.03 [M+H]⁺. ¹H NMR (400 MHz, CDCl₃) δ 5.98 (s, 1H), 4.65 (s, 2H), 4.11-4.04 (m, 4H), 3.49 (d, J=5.2 Hz, 2H), 3.29 (t, J=5.6 Hz, 2H), 2.11 (br s, 1H), 1.60 (br s, 1H).

To a stirred solution of (4,5,6,7-tetrahydropyrazolo[1,5-a]pyrazin-2-yl)methanol (22-4, 52.00 g, 339.67 mmol) in DCM (500 mL) was added di-tert-butyl dicarbonate (81.50 g, 373.6 mmol) at 0° C., and the mixture was allowed to stir at rt for 1 h. The mixture was diluted with water (500 mL) and extracted with DCM (3×500 mL). The combined organic layer was washed with brine (1 L), dried over Na₂SO₄, filtered and evaporated to afford the semi pure compound that was purified by silica gel column purification eluting with 10-20% EtOAc:PE to afford tert-butyl 2-(hydroxymethyl)-6,7-dihydropyrazolo[1,5-a]pyrazine-5(4H)-carboxylate (22-5, 60.0 g, 237.02 mmol, 70%) as a white solid. MS (LCMS) m/z 254.63 [M+H]⁺. ¹H NMR (400 MHz, CDCl₃) δ 6.06 (s, 1H), 4.64 (s, 2H), 4.63 (s, 2H), 4.13 (t, J=5.6 Hz, 2H), 3.86 (t, J=5.6 Hz, 2H), 3.14 (br s, 1H), 1.49 (s, 9H).

To a stirred solution of tert-butyl 2-(hydroxymethyl)-6,7-dihydropyrazolo[1,5-a]pyrazine-5(4H)-carboxylate (22-5, 3×20 g, 79.00 mmol) in DCM (200 mL) was added NBS (15.46 g, 86.91 mmol) at 0° C., and the mixture was stirred at rt for 1 h. The mixture was diluted with an aq. sat. NaHCO₃ solution (250 mL) and extracted with DCM (3×250 mL). The organic layer was separated, dried over Na₂SO₄, filtered and evaporated to give semi pure compound that was purified by silica gel column chromatography eluting with 20-30% EtOAc:PE to afford tert-butyl 3-bromo-2-(hydroxymethyl)-6,7-dihydropyrazolo[1,5-a]pyrazine-5(4H)-carboxylate (22-6, 53.0 g, 160.10 mmol, 67%) as a light brown solid. MS (LCMS) m/z 332.03 [M+H, M+3H]⁺. ¹H NMR (400 MHz, CDCl₃) δ 4.66 (s, 2H), 4.53 (s, 2H), 4.13 (t, J=5.2 Hz, 2H), 3.88 (t, J=5.2 Hz, 2H), 2.00 (br s, 1H), 1.51 (s, 9H).

To a stirred solution of tert-butyl 3-bromo-2-(hydroxymethyl)-6,7-dihydropyrazolo[1,5-a]pyrazine-5(4H)-carboxylate (22-6, 3×14 g, 42.29 mmol) in DMF (140 mL) was added NaH (60% in oil, 2.54 g, 63.43 mmol) at 0° C., and the mixture was stirred at rt for 20 min. 1-(Chloromethyl)-4-methoxybenzene (9.90 g, 63.43 mmol) and KI (712 mg, 4.29 mmol) were added, and the mixture was stirred at rt for 6 h. After completion of reaction, the reaction was quenched with a sat. aq. NH₄Cl solution (250 mL). The mixture was extracted with EtOAc (4×250 mL). The combined organic layer was washed with water (2×250 mL) and brine (250 mL), dried over Na₂SO₄ and concentrated under reduced pressure to give the semi pure compound that was purified by silica gel column chromatography eluting with 20-30% EtOAc:PE to afford the desired compound (35 g) that was mixed with the previous 75% LCMS purity (6.5 g). The mixture was stirred in pentane (200 mL) for 15 min, filtered and dried to give tert-butyl 3-bromo-2-(((4-methoxybenzyl)oxy)methyl)-6,7-dihydropyrazolo[1,5-a]pyrazine-5(4H)-carboxylate (22-7, 29.0 g, 64.29 mmol, 40% for aver all 53 g of used compound 22-6) as a white solid. MS (LCMS) m/z 454.05 [M+H, M+3H]⁺. ¹H NMR (400 MHz, CDCl₃) δ 7.31 (d, J=8.4 Hz, 2H), 6.87 (d, J=8.8 Hz, 2H), 4.56-4.50 (m, 4H), 4.48 (s, 2H), 4.14 (t, J=5.2 Hz, 2H), 3.86 (t, J=5.2 Hz, 2H), 3.80 (s, 3H), 1.50 (s, 9H).

To a stirred solution of tert-butyl 3-bromo-2-(((4-methoxybenzyl)oxy)methyl)-6,7-dihydropyrazolo[1,5-a]pyrazine-5(4H)-carboxylate (22-7, 6×5 g, 11.08 mmol) in THF (150 mL) was added n-BuLi (1.6 M in hexanes, 8.3 mL, 13.30 mmol) at −78° C., and the mixture was stirred at −78° C. for 50 min. 2-isopropoxy-4,4,5,5-tetramethyl-1,3,2-dioxaborolane (10.31 g, 55.4 mmol) was added at −78° C., and the mixture was stirred at −78° C. for 1 h. The solvents were evaporated. The mixture was diluted with EtOAc (200 mL), filtered through a Celite pad. The filtrate was evaporated to give the semi pure compound that was purified by silica gel column chromatography eluting with 10-20% EtOAc:PE to afford tert-butyl 2-(((4-methoxybenzyl)oxy)methyl)-3-(4,4,5,5-tetramethyl-1,3,2-dioxaborolan-2-yl)-6,7-dihydropyrazolo[1,5-a]pyrazine-5(4H)-carboxylate (22-8, 36 g, 72.10 mmol, 81%) as a clear viscous liquid. MS (LCMS) m/z 500.23 [M+H]⁺. ¹H NMR (400 MHz, CDCl₃) δ 7.32 (d, J=8.8 Hz, 2H), 6.85 (d, J=8.8 Hz, 2H), 4.76 (s, 2H), 4.63 (s, 2H), 4.58 (s, 2H), 4.18-4.12 (m, 2H), 3.84 (t, J=5.2 Hz, 2H), 3.79 (s, 3H), 1.49 (s, 9H), 1.27 (s, 12H).

In a seal tube, to a stirred solution of tert-butyl 2-(((4-methoxybenzyl)oxy)methyl)-3-(4,4,5,5-tetramethyl-1,3,2-dioxaborolan-2-yl)-6,7-dihydropyrazolo[1,5-a]pyrazine-5(4H)-carboxylate (22-8, 2.0 g, 4.02 mmol) in 1,4-dioxane (20 mL) were added methyl 7-bromo-6-chloro-3-(3-methoxy-3-oxopropyl)-1H-indole-2-carboxylate (18×1.00 g, 2.68 mmol) and Cs₂CO₃ (1.75 g, 5.36 mmol). The solution was degassed with Ar and dichloro[1 1′-bis(di-tert-butylphosphino)ferrocene] palladium(II) (105 mg, 0.161 mmol) was added. The solution was degassed again for 5 mins. The mixture was heated at 120° C. for 16 h. The mixture was diluted with 10% MeOH:DCM (50 mL) and passed through a Celite pad that was washed with 10% MeOH:DCM (2×50 mL). The solvent was evaporated to give the semi pure compound that was purified by silica gel column chromatography eluting with 10-30% EtOAc in PE to afford tert-butyl 3-(6-chloro-3-(3-methoxy-3-oxopropyl)-2-(methoxycarbonyl)-1H-indol-7-yl)-2-(((4-methoxybenzyl)oxy)methyl)-6,7-dihydropyrazolo[1,5-a]pyrazine-5(4H)-carboxylate (22-9, 18.0 g, 27.02 mmol, 40% for two steps) as a brown viscous liquid. MS (LCMS) m/z 667.17 [M+H]⁺. ¹H NMR (400 MHz, CDCl₃) δ 9.21 (br s, 1H), 7.66 (d, J=8.8 Hz, 1H), 7.31-7.22 (m, 1H), 7.01 (d, J=8.8 Hz, 2H), 6.75 (d, J=8.4 Hz, 2H), 4.41-4.39 (m, 4H), 4.31-4.28 (m, 2H), 4.18-4.11 (m, 2H), 3.80 (s, 2H), 3.77 (s, 3H), 3.75 (s, 3H), 3.67 (s, 3H), 3.48-3.38 (m, 2H), 2.72 (t, J=7.6 Hz, 2H), 1.40 (s, 9H).

To a stirred solution of methyl tert-butyl 3-(6-chloro-3-(3-methoxy-3-oxopropyl)-2-(methoxycarbonyl)-1H-indol-7-yl)-2-(((4-methoxybenzyl)oxy)methyl)-6,7-dihydropyrazolo[1,5-a]pyrazine-5(4H)-carboxylate (22-9, 2×8.0 g, 12.01 mmol) in dry DMF (80 mL) were added Cs₂CO₃ (5.87 g, 18.01 mmol) and Mel (3.41 g, 24.02 mmol), and the mixture was stirred at rt for 2 h. The reaction was quenched with water (200 mL) and extracted with EtOAc (3×200 mL). The organic layer was separated, dried over Na₂SO₄, filtered and evaporated to afford the semi pure compound that was purified by silica gel column chromatography eluting with another 5 g batch with 10-20% EtOAc in PE to afford tert-butyl 3-(6-chloro-3-(3-methoxy-3-oxopropyl)-2-(methoxycarbonyl)-1-methyl-1H-indol-7-yl)-2-(((4-methoxybenzyl)oxy)methyl)-6,7-dihydropyrazolo[1,5-a]pyrazine-5(4H)-carboxylate (22-10, 15.0 g, 3.63 mmol, 70% for 21 g batch altogether) as a dark brown viscous liquid. MS (LCMS) m/z 681.21 [M+H]⁺. ¹H NMR (400 MHz, CDCl₃) δ 7.64 (d, J=8.4 Hz, 1H), 7.29 (d, J=8.8 Hz, 1H), 6.88-6.85 (m, 2H), 6.69-6.65 (m, 2H), 4.51-4.22 (m, 8H), 3.97-3.84 (m, 5H), 3.78 (s, 3H), 3.68 (s, 3H), 3.51 (s, 3H), 3.40-3.29 (m, 2H), 2.67 (t, J=8.0 Hz, 2H), 1.44 (s, 9H).

To a stirred solution of tert-butyl 3-(6-chloro-3-(3-methoxy-3-oxopropyl)-2-(methoxycarbonyl)-1-methyl-1H-indol-7-yl)-2-(((4-methoxybenzyl)oxy)methyl)-6,7-dihydropyrazolo[1,5-a]pyrazine-5(4H)-carboxylate (22-10, 2×4.5 g, 6.62 mmol) in DCM (85.5 mL) and water (4.5 mL) was added 2,3-dichloro-5,6-dicyano-1,4-benzoquinone (DDQ) (1.80 mL, 7.95 mmol) at 0° C., and the mixture was stirred at rt for 3 h. The reaction was quenched with a sat. aq. NaHCO₃ solution (100 mL) and extracted with DCM (3×100 mL). The organic layer was separated and washed with water (3×150 mL) and brine (150 mL) dried over Na₂SO₄, filtered and evaporated to give the semi pure compound that was purified by silica gel column chromatography with another 6 g batch eluting with 20-50% EtOAc in PE to afford tert-butyl 3-(6-chloro-3-(3-methoxy-3-oxopropyl)-2-(methoxycarbonyl)-1-methyl-1H-indol-7-yl)-2-(hydroxymethyl)-6,7-dihydropyrazolo[1,5-a]pyrazine-5(4H)-carboxylate (22-11, 11.0 g, 19.64 mmol, 89% for 15 g batch altogether) as a dark brown viscous liquid. MS (LCMS) m/z 561.17 [M+H]⁺. ¹H NMR (400 MHz, CDCl₃) δ 7.65 (d, J=8.8 Hz, 1H), 7.27-7.24 (m, 1H), 4.55-4.45 (m, 2H), 4.40-4.31 (m, 2H), 4.28 (t, J=5.2 Hz, 2H), 3.92 (s, 3H), 3.92-3.85 (m, 2H), 3.68 (s, 3H), 3.55 (s, 3H), 3.34 (t, J=7.6 Hz, 2H), 2.66 (t, J=8.0 Hz, 2H), 1.81 (br s, 1H), 1.44 (s, 9H).

To a stirred solution of tert-butyl 3-(6-chloro-3-(3-methoxy-3-oxopropyl)-2-(methoxycarbonyl)-1-methyl-1H-indol-7-yl)-2-(hydroxymethyl)-6,7-dihydropyrazolo[1,5-a]pyrazine-5(4H)-carboxylate (22-11, 2×3.5 g, 6.25 mmol) in DCM (35 mL) under Ar was added SOCl₂ (884 mg, 7.50 mmol) at 0° C., and the mixture was stirred at rt for 30 min. The mixture was diluted with DCM (100 mL) and washed with a sat. NaHCO₃ solution (3×75 mL). The organic layer was separated, dried over Na₂SO₄, filtered and evaporated to afford semi pure tert-butyl 3-(6-chloro-3-(3-methoxy-3-oxopropyl)-2-(methoxycarbonyl)-1-methyl-1H-indol-7-yl)-2-(chloromethyl)-6,7-dihydropyrazolo[1,5-a]pyrazine-5(4H)-carboxylate (22-12, 7 g, 12.11 mmol, for two batches) as a dark brown viscous liquid that was used without further purification. MS (LCMS) m/z 579.13 [M+H]⁺. ¹H NMR (400 MHz, CDCl₃) δ 7.67 (d, J=8.8 Hz, 1H), 7.27-7.23 (m, 1H), 4.51-4.41 (m, 2H), 4.36-4.28 (m, 4H), 3.95 (s, 3H), 3.95-3.89 (m, 2H), 3.68 (s, 3H), 3.57 (s, 3H), 3.40-3.29 (m, 2H), 2.67 (t, J=8.0 Hz, 2H), 1.44 (s, 9H).

To a stirred solution of semi pure tert-butyl 3-(6-chloro-3-(3-methoxy-3-oxopropyl)-2-(methoxycarbonyl)-1-methyl-1H-indol-7-yl)-2-(chloromethyl)-6,7-dihydropyrazolo[1,5-a]pyrazine-5(4H)-carboxylate (22-12, 2×3.5 g, 6.05 mmol) in MeCN (35 mL) was added NaI (1.63 g, 10.90 mmol) at rt, and the mixture was heated to 80° C. for 6 h. The solvent was evaporated. The mixture was diluted with water (100 mL) and extracted with EtOAc (3×100 mL). The organic layer was separated, dried over Na₂SO₄, filtered and evaporated to afford semi pure tert-butyl 3-(6-chloro-3-(3-methoxy-3-oxopropyl)-2-(methoxycarbonyl)-1-methyl-1H-indol-7-yl)-2-(iodomethyl)-6,7-dihydropyrazolo[1,5-a]pyrazine-5(4H)-carboxylate (22-13, 8.0 g, 11.94 mmol, for two batches) as a dark brown viscous liquid that was used without further purification. MS (LCMS) m/z 671.06 [M+H]⁺. ¹H NMR (400 MHz, CDCl₃) δ 7.68 (d, J=8.8 Hz, 1H), 7.27 (d, J=8.4 Hz, 1H), 4.38-4.31 (m, 2H), 4.30-4.20 (m, 4H), 3.93 (s, 3H), 3.93-3.35 (m, 2H), 3.68 (s, 3H), 3.60 (s, 3H), 3.40-3.30 (m, 2H), 2.68 (t, J=8.0 Hz, 2H), 1.44 (s, 9H).

To a stirred solution of semi pure tert-butyl 3-(6-chloro-3-(3-methoxy-3-oxopropyl)-2-(methoxycarbonyl)-1-methyl-1H-indol-7-yl)-2-(iodomethyl)-6,7-dihydropyrazolo[1,5-a]pyrazine-5(4H)-carboxylate (22-13, 2×4.0 g, 5.97 mmol) in MeOH (20 mL), THF (8 mL) and K₂CO₃ (4.12 g, 29.85 mmol) were added. The solution was degassed with Ar for 10 min. In another round bottom flask, S-((5-(((4-(allyloxy)naphthalen-2-yl)thio)methyl)-1-methyl-1H-pyrazol-3-yl)methyl) ethanethioate (15) (2.377 g, 5.97 mmol) in MeOH (20 mL) and THF (8 mL) was degassed with Ar for 10 min, and this solution was added to previous mixture dropwise. The mixture was stirred at rt for 16 h. The solvent was evaporated. The mixture was diluted with water (200 mL) and extracted with EtOAc (3×250 mL). The organic layer was separated, dried over Na₂SO₄, filtered and evaporated to afford semi pure compound that was purified along with another 3 g batch by normal phase GRACE purification using 50-70% EtOAc in PE (3×) to afford tert-butyl 2-((((5-(((4-(allyloxy)naphthalen-2-yl)thio)methyl)-1-methyl-1H-pyrazol-3-yl)methyl)thio)methyl)-3-(6-chloro-3-(3-methoxy-3-oxopropyl)-2-(methoxycarbonyl)-1-methyl-1H-indol-7-yl)-6,7-dihydropyrazolo[1,5-a]pyrazine-5(4H)-carboxylate (22-14, 5.8 g, 6.46 mmol, 36% for three steps collective for 10 g batch) as a dark brown viscous liquid. MS (LCMS) m/z 899.27 [M+H]⁺. ¹H NMR (400 MHz, CDCl₃) δ 8.24-8.21 (m, 1H), 7.68-7.65 (m, 1H), 7.60 (d, J=8.8 Hz, 1H), 7.50-7.41 (m, 2H), 7.37 (s, 1H), 7.21 (d, J=8.4 Hz, 1H), 6.65 (d, J=1.2 Hz, 1H), 6.20-6.10 (m, 1H), 5.91 (s, 1H), 5.51-5.29 (m, 2H), 4.64-4.62 (m, 2H), 4.36-4.20 (m, 4H), 4.05 (s, 2H), 3.91 (s, 3H), 3.91-3.85 (m, 2H), 3.70 (s, 3H), 3.67 (s, 3H), 3.60-3.49 (m, 7H), 3.40-3.25 (m, 2H), 2.65 (t, J=8.0 Hz, 2H), 1.43 (s, 9H).

To a stirred solution of tert-butyl 2-((((5-(((4-(allyloxy)naphthalen-2-yl)thio)methyl)-1-methyl-1H-pyrazol-3-yl)methyl)thio)methyl)-3-(6-chloro-3-(3-methoxy-3-oxopropyl)-2-(methoxycarbonyl)-1-methyl-1H-indol-7-yl)-6,7-dihydropyrazolo[1,5-a]pyrazine-5(4H)-carboxylate (22-14, 4.8 g, 5.34 mmol) in DCM (50 mL) were added Pd(PPh₃)₄(124 mg, 0.107 mmol) and PhSiH₃ (692 mg, 6.41 mmol) at rt, and the mixture was stirred for 2 h. The volatiles were evaporated to give the semi pure compound that was purified along with another 1 g batch by normal phase GRACE purification using 40-70% EtOAc in PE to afford tert-butyl 3-(6-chloro-3-(3-methoxy-3-oxopropyl)-2-(methoxycarbonyl)-1-methyl-1H-indol-7-yl)-2-((((5-(((4-hydroxynaphthalen-2-yl)thio)methyl)-1-methyl-1H-pyrazol-3-yl)methyl)thio)methyl)-6,7-dihydropyrazolo[1,5-a]pyrazine-5(4H)-carboxylate (22-15, 5.5 g, 6.4 mmol, 99% altogether for 5.8 g batch) as a viscous brown liquid. MS (LCMS) m/z 859.20 [M+H]⁺. ¹H NMR (400 MHz, CDCl₃) δ 9.50 (br s, 1H), 8.28-8.21 (m, 1H), 7.79-7.75 (m, 1H), 7.70-7.61 (m, 2H), 7.51-7.43 (m, 3H), 6.57 (d, J=1.6 Hz, 1H), 5.97 (s, 1H), 4.40-4.28 (m, 4H), 3.95 (s, 3H), 3.92 (s, 2H), 3.92-3.81 (m, 2H), 3.70 (s, 3H), 3.65-3.55 (m, 5H), 3.55-3.49 (m, 5H), 3.40-3.30 (m, 2H), 2.69 (t, J=8.4 Hz, 2H), 1.44 (s, 9H).

To a suspension of tert-butyl 3-(6-chloro-3-(3-methoxy-3-oxopropyl)-2-(methoxycarbonyl)-1-methyl-1H-indol-7-yl)-2-((((5-(((4-hydroxynaphthalen-2-yl)thio)methyl)-1-methyl-1H-pyrazol-3-yl)methyl)thio)methyl)-6,7-dihydropyrazolo[1,5-a]pyrazine-5(4H)-carboxylate (22-15, 1.0 g, 1.16 mmol) in dry THF (6 mL) was added BH₃.THF (1.0 M in THF, 14.0 mL, 14.0 mmol) dropwise at 0° C. The temperature was raised to rt and stirred for 16 h. The reaction was quenched with MeOH (5 mL) and 6N HCl (5 mL) at 0° C. The mixture was stirred for 10 min and then at rt for 20 min. The mixture was further diluted with water (25 mL) and extracted with 10% MeOH in DCM (3×30 mL). The organic layer was separated and further washed with a sat. NaHCO₃ solution (3×25 mL). The organic layer was separated, dried over Na₂SO₄, filtered and evaporated to give the semi pure compound that was purified by normal phase GRACE using 50-70% EtOAc in PE to afford tert-butyl 3-(6-chloro-3-(3-hydroxypropyl)-2-(methoxycarbonyl)-1-methyl-1H-indol-7-yl)-2-((((5-(((4-hydroxynaphthalen-2-yl)thio)methyl)-1-methyl-1H-pyrazol-3-yl)methyl)thio)methyl)-6,7-dihydropyrazolo[1,5-a]pyrazine-5(4H)-carboxylate (22-16, 480 mg, 0.57 mmol, 49%) as a clear viscous liquid. MS (LCMS) m/z 831.24 [M+H]⁺. ¹H NMR (400 MHz, CDCl₃) δ 9.51 (br s, 1H), 8.27-8.23 (m, 1H), 7.77-7.73 (m, 1H), 7.66-7.62 (m, 2H), 7.53-7.45 (m, 2H), 7.26-7.22 (m, 1H), 6.56 (s, 1H), 5.96 (s, 1H), 4.44-4.28 (m, 4H), 3.95 (s, 3H), 3.92 (s, 2H), 3.92-3.85 (m, 2H), 3.71-3.58 (m, 4H), 3.56 (m, 3H), 3.52-3.45 (m, 5H), 3.17 (t, J=7.2 Hz, 2H), 2.21 (br s, 1H), 2.05-1.90 (m, 2H), 1.44 (s, 9H).

To a stirred solution of TPP (442 mg, 1.686 mmol) in toluene (10 mL) was added a solution of di-tert-butyl diazene-1,2-dicarboxylate (388 mg, 1.686 mmol), tert-butyl 3-(6-chloro-3-(3-hydroxypropyl)-2-(methoxycarbonyl)-1-methyl-1H-indol-7-yl)-2-((((5-(((4-hydroxynaphthalen-2-yl)thio)methyl)-1-methyl-1H-pyrazol-3-yl)methyl)thio)methyl)-6,7-dihydropyrazolo[1,5-a]pyrazine-5(4H)-carboxylate (22-16, 3×700 mg, 0.843 mmol) in toluene (11 mL) and THF (1.4 mL), and the mixture was stirred at rt for 2 h. The reaction was quenched with water (20 mL) and extracted with EtOAc (3×25 mL). The organic layer was dried over Na₂SO₄, filtered, concentrated to give the semi pure compound that was purified by RP prep HPLC. The fractions were evaporated to afford 2⁵-(tert-butyl) 1²-methyl (Z)-1⁶-chloro-1¹,6¹-dimethyl-2⁴,2⁵,2⁶,2⁷-tetrahydro-1¹H,6¹H-10-oxa-4,8-dithia-2(3,2)-pyrazolo[1,5-a]pyrazina-1(7,3)-indola-6(3,5)-pyrazola-9(3,1)-naphthalenacyclotridecaphane-1²,2⁵-dicarboxylate (22-17, 700 mg, 0.86 mmol, 34% for three batches) as an off white solid. MS (LCMS) m/z 813.27 [M+H]⁺. ¹H NMR (400 MHz, CDCl₃) δ 8.33-8.30 (m, 1H), 7.74-7.71 (m, 1H), 7.56-7.49 (m, 4H), 6.94 (d, J=8.4 Hz, 1H), 6.24 (d, J=1.6 Hz, 1H), 4.90 (s, 1H), 4.40-4.30 (m, 2H), 4.28-4.19 (m, 2H), 3.96-3.85 (m, 7H), 3.85-3.75 (m, 1H), 3.73 (s, 3H), 3.68 (s, 3H), 3.67-3.60 (m, 1H), 3.58-3.45 (m, 2H), 3.31-3.20 (m, 2H), 3.10-3.04 (m, 1H), 3.70-3.61 (m, 1H), 2.50-2.38 (m, 1H), 2.27-2.15 (m, 1H), 1.43 (s, 9H).

2⁵-(tert-butyl) 1²-methyl (Z)-1⁶-chloro-1¹,6¹-dimethyl-2⁴,2⁵,2⁶,2⁷-tetrahydro-1¹H,6¹H-10-oxa-4,8-dithia-2(3,2)-pyrazolo[1,5-a]pyrazina-1(7,3)-indola-6(3,5)-pyrazola-9(3,1)-naphthalenacyclotridecaphane-1²,2⁵-dicarboxylate (22-17, 350 mg, LCMS 90%) was purified by SFC purification to afford 22-17-peak-1 (120 mg) as an off white solid with 94.27% of LCMS purity (chiral HPLC:99.94%) and 22-17-peak-2 (120 mg) as an off white solid with 84.08% of LCMS purity (chiral HPLC:99.60%). These two peaks were separately used for the next steps to get respective final compound.

22-17-peak-1: MS (LCMS) m/z 813.24 [M+H]⁺. ¹H NMR (400 MHz, CDCl₃) δ 8.33-8.30 (m, 1H), 7.74-7.71 (m, 1H), 7.55-7.49 (m, 4H), 6.94 (d, J=8.4 Hz, 1H), 6.24 (d, J=1.2 Hz, 1H), 4.90 (s, 1H), 4.40-4.30 (m, 2H), 4.28-4.19 (m, 2H), 3.97-3.85 (m, 7H), 3.85-3.75 (m, 1H), 3.73 (s, 3H), 3.68 (s, 3H), 3.67-3.60 (m, 1H), 3.58-3.45 (m, 2H), 3.31-3.20 (m, 2H), 3.10-3.01 (m, 1H), 3.70-3.62 (m, 1H), 2.50-2.39 (m, 1H), 2.26-2.15 (m, 1H), 1.43 (s, 9H).

22-17-peak-2: MS (LCMS) m/z 813.27 [M+H]⁺. ¹H NMR (400 MHz, CDCl₃) δ 8.33-8.30 (m, 1H), 7.74-7.71 (m, 1H), 7.56-7.49 (m, 4H), 6.94 (d, J=8.8 Hz, 1H), 6.24 (d, J=1.2 Hz, 1H), 4.90 (s, 1H), 4.40-4.30 (m, 2H), 4.28-4.19 (m, 2H), 3.96-3.85 (m, 7H), 3.85-3.75 (m, 1H), 3.73 (s, 3H), 3.68 (s, 3H), 3.67-3.60 (m, 1H), 3.58-3.45 (m, 2H), 3.31-3.20 (m, 2H), 3.10-3.02 (m, 1H), 3.70-3.61 (m, 1H), 2.50-2.38 (m, 1H), 2.27-2.15 (m, 1H), 1.43 (s, 9H).

To a stirred solution of methyl 2⁵-(tert-butyl) 1²-methyl (Z)-1⁶-chloro-1¹,6¹-dimethyl-2⁴,2⁵,2⁶,2⁷-tetrahydro-1¹H,6¹H-10-oxa-4,8-dithia-2(3,2)-pyrazolo[1,5-a]pyrazina-1(7,3)-indola-6(3,5)-pyrazola-9(3,1)-naphthalenacyclotridecaphane-1²,2⁵-dicarboxylate (22-17-peak-1, 120 mg, 0.15 mmol, LCMS 94%) in MeOH:THF:H₂O (10:10:3, 5 mL) was added LiOH.H₂O (63 mg, 1.5 mmol) at 0° C., and the mixture was stirred at rt for 2 h. Two portions of LiOH.H₂O (31.5 mg, 0.75 mmol) was added at 0° C., and the mixture was stirred at rt for 1 h. The solvent was evaporated. The mixture was diluted with water (2 mL), and the aqueous layer was acidified to pH 2 using 1N aq. HCl. The mixture was stirred for 1 min, and solid precipitation was observed. The mixture was filtered, washed with water and dried to afford (Z)-2⁵-(tert-butoxycarbonyl)-1⁶-chloro-1¹,6¹-dimethyl-2⁴,2⁵,2⁶,2⁷-tetrahydro-1¹H,6¹H-10-oxa-4,8-dithia-2(3,2)-pyrazolo[1,5-a]pyrazina-1(7,3)-indola-6(3,5)-pyrazola-9(3,1)-naphthalenacyclotridecaphane-1²-carboxylic acid (22A, 100 mg, 0.125 mmol, 83%) as an off white solid. MS (LCMS) m/z 799.24 [M+H]⁺. ¹H NMR (400 MHz, DMSO-d₆) δ 13.39 (br s, 1H), 8.12-8.08 (m, 1H), 7.90 (d, J=8.4 Hz, 1H), 7.74-7.71 (m, 1H), 7.52-7.43 (m, 2H), 7.38 (s, 1H), 7.16 (d, J=8.4 Hz, 1H), 6.67 (s, 1H), 4.76 (s, 1H), 4.30-4.08 (m, 7H), 3.90-3.79 (m, 3H), 3.71 (s, 3H), 3.53 (s, 3H), 3.50-3.47 (m, 1H), 3.25-3.20 (m, 1H), 3.15-3.06 (m, 2H), 2.91 (d, J=14.0 Hz, 1H), 2.44-2.15 (m, 2H), 1.36 (s, 9H).

To a stirred solution of methyl 2⁵-(tert-butyl) 1²-methyl (Z)-1⁶-chloro-1¹,6¹-dimethyl-2⁴,2⁵,2⁶,2⁷-tetrahydro-1¹H,6¹H-10-oxa-4,8-dithia-2(3,2)-pyrazolo[1,5-a]pyrazina-1(7,3)-indola-6(3,5)-pyrazola-9(3,1)-naphthalenacyclotridecaphane-1²,2⁵-dicarboxylate (22-17-peak-2, 120 mg, 0.15 mmol, LCMS 84%) in MeOH:THF:H₂O (10:10:3, 5 mL) was added LiOH.H₂O (63 mg, 1.5 mmol) at 0° C., and the mixture was stirred at rt for 2 h. Two portions of LiOH.H₂O (31.5 mg, 0.75 mmol) was added at 0° C., and the mixture was stirred at rt for 1 h. The solvent was evaporated, and the mixture was diluted with water (2 mL). The aqueous layer was acidified to pH 2 using 1N aq. HCl, and the mixture was stirred for 1 min. A solid precipitation was observed. The mixture was filtered, washed with water and dried to give (Z)-2⁵-(tert-butoxycarbonyl)-1⁶-chloro-1¹,6¹-dimethyl-2⁴,2⁵,2⁶,2⁷-tetrahydro-1¹H,6¹H-10-oxa-4,8-dithia-2(3,2)-pyrazolo[1,5-a]pyrazina-1(7,3)-indola-6(3,5)-pyrazola-9(3,1)-naphthalenacyclotridecaphane-1²-carboxylic acid (22B, 100 mg, 81% LCMS purity). The compound was further purified by silica gel prep TLC using 5% MeOH:DCM to afford (Z)-2⁵-(tert-butoxycarbonyl)-1⁶-chloro-1¹,6¹-dimethyl-2⁴,2⁵,2⁶,2⁷-tetrahydro-1¹H,6¹H-10-oxa-4,8-dithia-2(3,2)-pyrazolo[1,5-a]pyrazina-1(7,3)-indola-6(3,5)-pyrazola-9(3,1)-naphthalenacyclotridecaphane-1²-carboxylic acid (22B, 60 mg, 0.075 mmol, 50%) as an off white solid. MS (LCMS) m/z 799.24 [M+H]⁺. ¹H NMR (400 MHz, DMSO-d₆) δ 13.40 (br s, 1H), 8.09 (d, J=7.6 Hz, 1H), 7.84 (d, J=8.4 Hz, 1H), 7.71 (d, J=7.2 Hz, 1H), 7.51-7.43 (m, 2H), 7.37 (s, 1H), 7.13 (d, J=8.8 Hz, 1H), 6.70 (s, 1H), 4.82 (s, 1H), 4.40-4.05 (m, 7H), 3.91-3.75 (m, 3H), 3.71 (s, 3H), 3.52 (s, 3H), 3.50-3.40 (m, 1H), 3.25-3.20 (m, 1H), 3.20-3.00 (m, 2H), 2.92 (d, J=14.0 Hz, 1H), 2.44-2.15 (m, 2H), 1.36 (s, 9H).

The absolute stereochemistry of compounds (22A) and (22B) is arbitrarily assigned.

### Example 23

### (Z)-1⁶-Chloro-1¹,6¹-dimethyl-2⁴,2⁵,2⁶,2⁷-tetrahydro-1¹H,6¹H-10-oxa-4,8-dithia-2(3,2)-pyrazolo[1,5-a]pyrazina-1(7,3)-indola-6(3,5)-pyrazola-9(3,1)-naphthalenacyclotridecaphane-1²-carboxylic acid

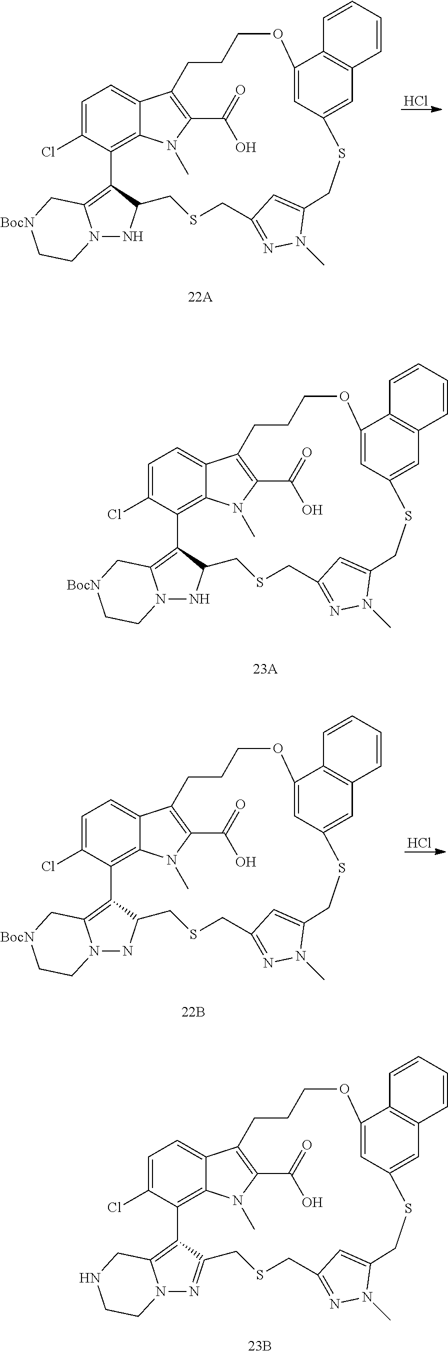

Compounds 23A and 23B were prepared from the corresponding 22A and 22B as following.

To a stirred solution of (Z)-2⁵-(tert-butoxycarbonyl)-1⁶-chloro-1¹,6¹-dimethyl-2⁴,2⁵,2⁶,2⁷-tetrahydro-1¹H,6¹H-10-oxa-4,8-dithia-2(3,2)-pyrazolo[1,5-a]pyrazina-1(7,3)-indola-6(3,5)-pyrazola-9(3,1)-naphthalenacyclotridecaphane-1²-carboxylic acid (22A, 65 mg, 0.081 mmol) in DCM (0.65 mL) was added 4N HCl in dioxane (0.65 mL) at 0° C., and the mixture was stirred at rt for 4 h. The solvent was evaporated, triturated with pentane:ether and lyophilized with MeCN:H₂O to give (Z)-1⁶-chloro-1¹,6¹-dimethyl-2⁴,2⁵,2⁶,2⁷-tetrahydro-1¹H,6¹H-10-oxa-4,8-dithia-2(3,2)-pyrazolo[1,5-a]pyrazina-1(7,3)-indola-6(3,5)-pyrazola-9(3,1)-naphthalenacyclotridecaphane-1²-carboxylic acid as the HCl salt (23A, 65 mg, quantitative) as an off white solid. MS (LCMS) m/z 699.17 [M+H]⁺. ¹H NMR (400 MHz, DMSO-d₆) δ 13.40 (br s, 1H), 9.67 (br s, 1H), 9.51 (br s, 1H), 8.11 (d, J=8.0 Hz, 1H), 7.89 (d, J=8.4 Hz, 1H), 7.73 (d, J=7.2 Hz, 1H), 7.52-7.44 (m, 2H), 7.40 (s, 1H), 7.13 (d, J=8.4 Hz, 1H), 6.64 (s, 1H), 4.79 (s, 1H), 4.40-4.31 (m, 2H), 4.25 (s, 2H), 4.20-4.00 (m, 3H), 3.91-3.80 (m, 1H), 3.80-3.70 (m, 2H), 3.69 (s, 3H), 3.56 (s, 3H), 3.50-3.42 (m, 2H), 3.25 (d, J=12.8 Hz, 1H), 3.15-3.06 (m, 2H), 2.90 (d, J=14.0 Hz, 1H), 2.42-2.14 (m, 2H).

To a stirred solution of (Z)-2⁵-(tert-butoxycarbonyl)-1⁶-chloro-1¹,6¹-dimethyl-2⁴,2⁵,2⁶,2⁷-tetrahydro-1¹H,6¹H-10-oxa-4,8-dithia-2(3,2)-pyrazolo[1,5-a]pyrazina-1(7,3)-indola-6(3,5)-pyrazola-9(3,1)-naphthalenacyclotridecaphane-1²-carboxylic acid (22B, 35 mg, 0.044 mmol) in DCM (0.35 mL) was added 4N HCl in dioxane (0.35 mL) at 0° C., and the mixture was stirred at rt for 4 h. The solvent was evaporated, triturated with pentane:ether and lyophilized with MeCN:H₂O to give (Z)-1⁶-chloro-1¹,6¹-dimethyl-2⁴,2⁵,2⁶,2⁷-tetrahydro-1¹H,6¹H-10-oxa-4,8-dithia-2(3,2)-pyrazolo[1,5-a]pyrazina-1(7,3)-indola-6(3,5)-pyrazola-9(3,1)-naphthalenacyclotridecaphane-1²-carboxylic acid as the HCl salt (23B, 35 mg, quantitative) as an off white solid. MS (LCMS) m/z 699.17 [M+H]⁺. ¹H NMR (400 MHz, DMSO-d₆) δ 13.40 (br s, 1H), 9.92 (br s, 1H), 9.67 (br s, 1H), 8.11 (d, J=8.8 Hz, 1H), 7.89 (d, J=8.4 Hz, 1H), 7.73 (d, J=7.6 Hz, 1H), 7.52-7.44 (m, 2H), 7.40 (s, 1H), 7.13 (d, J=8.4 Hz, 1H), 6.64 (d, J=1.2 Hz, 1H), 4.79 (s, 1H), 4.40-4.32 (m, 2H), 4.25 (s, 2H), 4.19-4.00 (m, 3H), 3.91-3.80 (m, 1H), 3.80-3.70 (m, 2H), 3.70 (s, 3H), 3.56 (s, 3H), 3.50-3.42 (m, 2H), 3.25 (d, J=13.2 Hz, 1H), 3.18-3.07 (m, 2H), 2.90 (d, J=14.0 Hz, 1H), 2.42-2.16 (m, 2H).

The absolute stereochemistry of compounds (23A) and (23B) is arbitrarily assigned.

### Example 24

### (Z)-2⁵-Acetyl-1⁶-chloro-1¹,6¹-dimethyl-2⁴,2⁵,2⁶,2⁷-tetrahydro-1¹H,6¹H-10-oxa-4,8-dithia-2(3,2)-pyrazolo[1,5-a]pyrazina-1(7,3)-indola-6(3,5)-pyrazola-9(3,1)-naphthalenacyclotridecaphane-1²-carboxylic acid

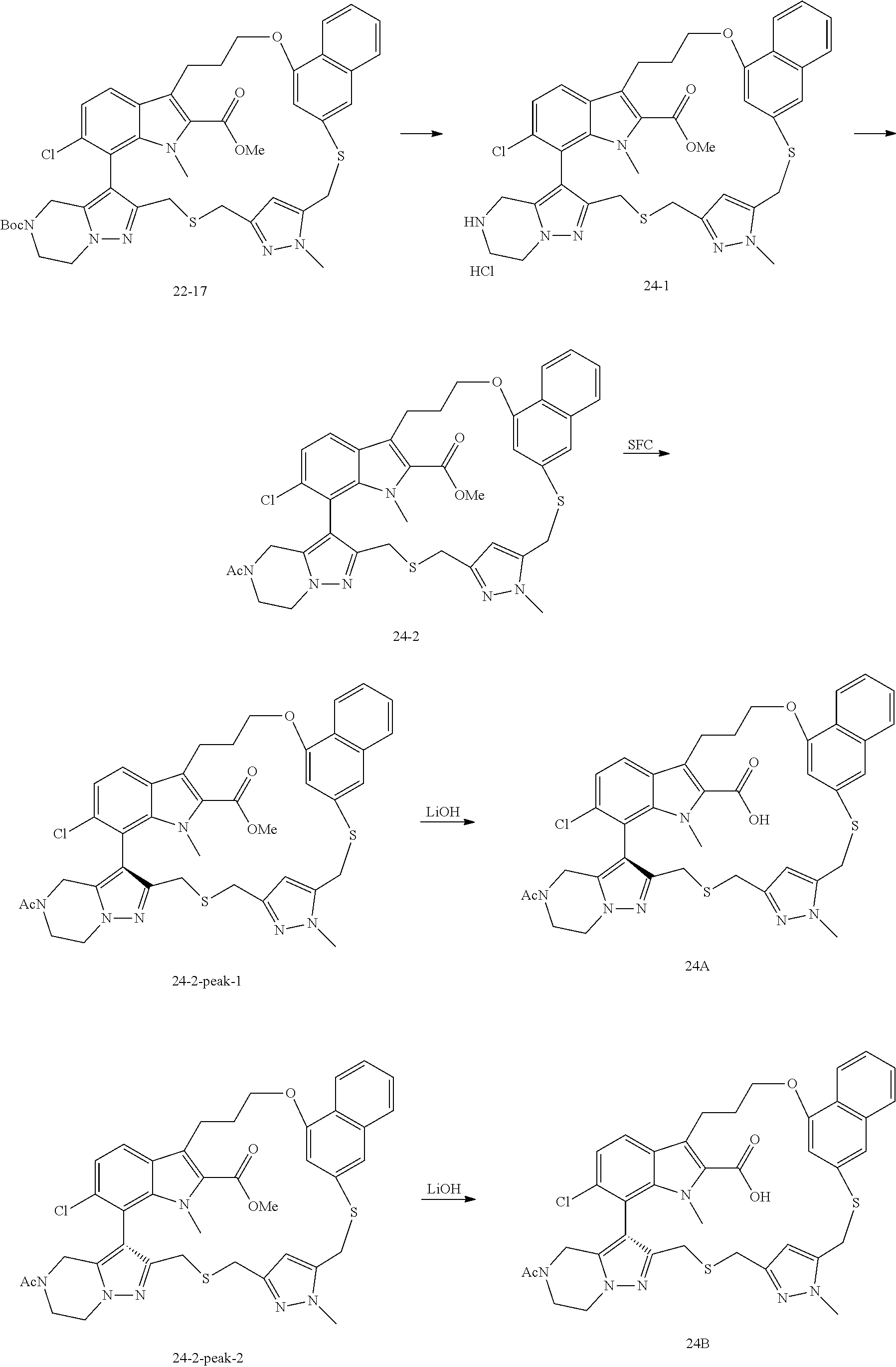

Compounds 24A and 24B were prepared by following procedure starting from 22-17 (example 22).

To a stirred solution of 2⁵-(tert-butyl) 1²-methyl (Z)-1⁶-chloro-1¹,6¹-dimethyl-2⁴,2⁵,2⁶,2⁷-tetrahydro-1¹H,6¹H-10-oxa-4,8-dithia-2(3,2)-pyrazolo[1,5-a]pyrazina-1(7,3)-indola-6(3,5)-pyrazola-9(3,1)-naphthalenacyclotridecaphane-1²,2⁵-dicarboxylate (22-17, 350 mg, 0.431 mmol) in DCM (3.5 mL) was added 4N HCl in dioxane (3.5 mL) at 0° C., and the mixture was stirred at rt for 4 h. The solvent was evaporated, triturated with pentane:ether to give methyl (Z)-1⁶-chloro-1¹,6¹-dimethyl-2⁴,2⁵,2⁶,2⁷-tetrahydro-1¹H,6¹H-10-oxa-4,8-dithia-2(3,2)-pyrazolo[1,5-a]pyrazina-1(7,3)-indola-6(3,5)-pyrazola-9(3,1)-naphthalenacyclotridecaphane-1²-carboxylate as the HCl salt (24-1, 350 mg, quantitative) as an off white solid. MS (LCMS) m/z 713.17 [M+H]⁺. ¹H NMR (400 MHz, DMSO-d₆) δ 10.28 (br s, 1H), 9.93 (br s, 1H), 8.13-8.10 (m, 1H), 7.92 (d, J=8.8 Hz, 1H), 7.75-7.72 (m, 1H), 7.53-7.45 (m, 2H), 7.42 (s, 1H), 7.17 (d, J=8.8 Hz, 1H), 6.61 (d, J=1.2 Hz, 1H), 4.84 (s, 1H), 4.43-4.30 (m, 2H), 4.30-4.20 (m, 2H), 4.19-3.99 (m, 3H), 3.84 (s, 3H), 3.84-3.70 (m, 2H), 3.67 (s, 3H), 3.65-3.60 (m, 1H), 3.57 (s, 3H), 3.50-3.38 (m, 2H), 3.24 (d, J=13.2 Hz, 1H), 3.19-3.10 (m, 1H), 3.02 (d, J=14.0 Hz, 1H), 2.91 (d, J=14.0 Hz, 1H), 2.40-2.30 (m, 1H), 2.29-2.15 (m, 1H).

To a stirred solution of methyl (Z)-1⁶-chloro-1¹,6¹-dimethyl-2⁴,2⁵,2⁶,2⁷-tetrahydro-1¹H,6¹H-10-oxa-4,8-dithia-2(3,2)-pyrazolo[1,5-a]pyrazina-1(7,3)-indola-6(3,5)-pyrazola-9(3,1)-naphthalenacyclotridecaphane-1²-carboxylate as HCl salt (24-1, 350 mg, 0.49 mmol) in DCM (3.5 mL) were added Et₃N (149 mg, 1.47 mmol), DMAP (3 mg, 0.0245 mmol) and Ac₂O (152 mg, 1.47 mmol) at 0° C., and the mixture was stirred at rt for 4 h. The mixture was diluted with DCM (20 mL), and washed with water (2×20 mL), 1N HCl (2×20 mL) and brine (20 mL). The organic layer was separated, dried over Na₂SO₄, filtered and evaporated to give the semi pure compound that was purified triturated with pentane and ether to get methyl (Z)-2⁵-acetyl-1⁶-chloro-1¹,6¹-dimethyl-2⁴,2⁵,2⁶,2⁷-tetrahydro-1¹H,6¹H-10-oxa-4,8-dithia-2(3,2)-pyrazolo[1,5-a]pyrazina-1(7,3)-indola-6(3,5)-pyrazola-9(3,1)-naphthalenacyclotridecaphane-1²-carboxylate (24-2, 320 mg, 0.424 mmol, 86%) a white solid that was submitted for SFC separation of the enantiomers. MS (LCMS) m/z 755.21 [M+H]⁺. ¹H NMR (400 MHz, CDCl₃) δ 8.33-8.30 (m, 1H), 7.76-7.50 (m, 1H), 7.60-7.49 (m, 4H), 6.95 (dd, J=20.8, 8.4 Hz, 1H), 6.24 (s, 1H), 4.92 (d, J=21.6 Hz, 1H), 4.60-4.20 (m, 4H), 3.99-3.75 (m, 8H), 3.73 (s, 3H), 3.67 (s, 3H), 3.70-3.60 (m, 1H), 3.55-3.45 (m, 2H), 3.32-3.20 (m, 2H), 3.10-3.00 (m, 1H), 2.67 (d, J=13.6 Hz, 1H), 2.50-2.39 (m, 1H), 2.28-2.15 (m, 1H), 2.20 & 2.05 (two singles, 3H).

Methyl (Z)-2⁵-acetyl-1⁶-chloro-1¹,6¹-dimethyl-2⁴,2⁵,2⁶,2⁷-tetrahydro-1¹H,6¹H-10-oxa-4,8-dithia-2(3,2)-pyrazolo[1,5-a]pyrazina-1(7,3)-indola-6(3,5)-pyrazola-9(3,1)-naphthalenacyclotridecaphane-1²-carboxylate (24-2, 320 mg, LCMS 93%) was purified by SFC purification to afford 24-2-peak-1 (120 mg) as an off white solid with 99.44% of LCMS purity (chiral HPLC:99.78%) and 24-2-peak-2 (120 mg) as an off white solid with 99.68% of LCMS purity (chiral HPLC:99.92%). These two peaks were separately used for the next steps to get respective final compound.

24-2-peak-1: MS (LCMS) m/z 755.17 [M+H]⁺. ¹H NMR (400 MHz, CDCl₃) δ 8.33-8.30 (m, 1H), 7.76-7.50 (m, 1H), 7.60-7.49 (m, 4H), 6.95 (dd, J=20.8, 8.4 Hz, 1H), 6.24 (s, 1H), 4.92 (d, J=22.0 Hz, 1H), 4.55-4.20 (m, 4H), 3.99-3.75 (m, 8H), 3.73 (s, 3H), 3.71 (s, 3H), 3.70-3.60 (m, 1H), 3.55-3.45 (m, 2H), 3.32-3.20 (m, 2H), 3.10-2.99 (m, 1H), 2.67 (d, J=13.6 Hz, 1H), 2.50-2.39 (m, 1H), 2.28-2.15 (m, 1H), 2.20 & 2.05 (two singles, 3H).

24-2-peak-2: MS (LCMS) m/z 755.17 [M+H]⁺. ¹H NMR (400 MHz, CDCl₃) δ 8.33-8.30 (m, 1H), 7.76-7.50 (m, 1H), 7.60-7.49 (m, 4H), 6.95 (dd, J=20.8, 8.4 Hz, 1H), 6.24 (s, 1H), 4.92 (d, J=21.6 Hz, 1H), 4.55-4.20 (m, 4H), 3.99-3.75 (m, 8H), 3.73 (s, 3H), 3.71 (s, 3H), 3.70-3.60 (m, 1H), 3.55-3.45 (m, 2H), 3.32-3.20 (m, 2H), 3.10-2.99 (m, 1H), 2.67 (d, J=13.6 Hz, 1H), 2.50-2.39 (m, 1H), 2.28-2.15 (m, 1H), 2.20 & 2.05 (two singles, 3H).

To a stirred solution of methyl (Z)-2⁵-acetyl-1⁶-chloro-1¹,6¹-dimethyl-2⁴,2⁵,2⁶,2⁷-tetrahydro-1¹H,6¹H-10-oxa-4,8-dithia-2(3,2)-pyrazolo[1,5-a]pyrazina-1(7,3)-indola-6(3,5)-pyrazola-9(3,1)-naphthalenacyclotridecaphane-1²-carboxylate (24-2-peak-1, 120 mg, 0.16 mmol, LCMS 99%) in MeOH:THF:H₂O (2:2:1, 2 mL) was added LiOH.H₂O (33.6 mg, 0.8 mmol) at 0° C., and the mixture was stirred at rt for 3 h. Another portion of LiOH.H₂O (16.8 mg, 0.4 mmol) was added at 0° C. and stirred at RT for 3 h. The solvent was evaporated, and the mixture was diluted with water (2 mL). The aqueous layer was acidified to pH 2 using 1N aq. HCl, and the mixture was stirred for 1 min. A solid precipitation was observed. The mixture was filtered, washed with water and dried. The compound purified by silica gel prep-TLC (5% MeOH/DCM) to get (Z)-2⁵-acetyl-1⁶-chloro-1¹,6¹-dimethyl-2⁴,2⁵,2⁶,2⁷-tetrahydro-1¹H,6¹H-10-oxa-4,8-dithia-2(3,2)-pyrazolo[1,5-a]pyrazina-1(7,3)-indola-6(3,5)-pyrazola-9(3,1)-naphthalenacyclotridecaphane-1²-carboxylic acid (24A, 70 mg, 0.095 mmol, 59%) as an off white solid. MS (LCMS) m/z 741.11 [M+H]⁺. ¹H NMR (400 MHz, DMSO-d₆) δ 13.30 (br s, 1H), 8.09 (d, J=7.6 Hz, 1H), 7.89-7.80 (m, 1H), 7.71 (d, J=8.4 Hz, 1H), 7.50-7.43 (m, 2H), 7.37 (d, J=4.0 Hz, 1H), 7.13 (d, J=8.4 Hz, 1H), 6.70 (s, 1H), 4.86 & 4.80 (two singles, 1H), 4.45-3.82 (m, 10H), 3.71 (s, 3H), 3.55-3.40 (m, 5H), 3.28-3.20 (m, 1H), 3.18-3.02 (m, 2H), 2.96-2.90 (m, 1H), 2.40-2.20 (m, 2H), 2.11 & 1.95 (two singles, 3H).

To a stirred solution of methyl (Z)-2⁵-acetyl-1⁶-chloro-1¹,6¹-dimethyl-2⁴,2⁵,2⁶,2⁷-tetrahydro-1¹H,6¹H-10-oxa-4,8-dithia-2(3,2)-pyrazolo[1,5-a]pyrazina-1(7,3)-indola-6(3,5)-pyrazola-9(3,1)-naphthalenacyclotridecaphane-1²-carboxylate (24-2-peak-2, 120 mg, 0.16 mmol, LCMS 99%) in MeOH:THF:H₂O (2:2:1, 2 mL) was added LiOH.H₂O (101 mg, 2.4 mmol) at 0° C., and the mixture was stirred at rt for 6 h. The solvent was evaporated, and the mixture was diluted with water (2 mL). The aqueous layer was acidified to pH 2 using 1N aq. HCl, and the mixture was stirred for 1 min. A solid precipitation was observed. The mixture was filtered, washed with water and dried. The compound purified by silica gel prep-TLC (5% MeOH/DCM) to get (Z)-2⁵-acetyl-1⁶-chloro-1¹,6¹-dimethyl-2⁴,2⁵,2⁶,2⁷-tetrahydro-1¹H,6¹H-10-oxa-4,8-dithia-2(3,2)-pyrazolo[1,5-a]pyrazina-1(7,3)-indola-6(3,5)-pyrazola-9(3,1)-naphthalenacyclotridecaphane-1²-carboxylic acid (24B, 40 mg, 0.054 mmol, 34%) as an off white solid. MS (LCMS) m/z 741.08 [M+H]⁺. ¹H NMR (400 MHz, DMSO-d₆) δ 13.30 (br s, 1H), 8.08 (d, J=7.6 Hz, 1H), 7.85-7.72 (m, 1H), 7.71 (d, J=8.4 Hz, 1H), 7.50-7.41 (m, 2H), 7.35 (s, 1H), 7.10 (d, J=8.4 Hz, 1H), 6.73 (s, 1H), 4.86 & 4.80 (two singles, 1H), 4.45-3.82 (m, 10H), 3.72 (s, 3H), 3.52-3.40 (m, 5H), 3.28-3.20 (m, 1H), 3.18-3.05 (m, 2H), 2.96-2.90 (m, 1H), 2.35-2.20 (m, 2H), 2.11 & 1.96 (two singles, 3H).

The absolute stereochemistry of compounds (24A) and (24B) is arbitrarily assigned.

### Example 25

### (Z)-1⁶-Chloro-1¹,6¹-dimethyl-2⁵,2⁶-dihydro-1¹H,2⁴H,6¹H-10-oxa-4-aza-1(7,3)-indola-2(3,2)-pyrrolo[1,2-b]pyrazola-6(3,5)-pyrazola-9(3,1)-naphthalenacyclotridecaphane-1²-carboxylic acid

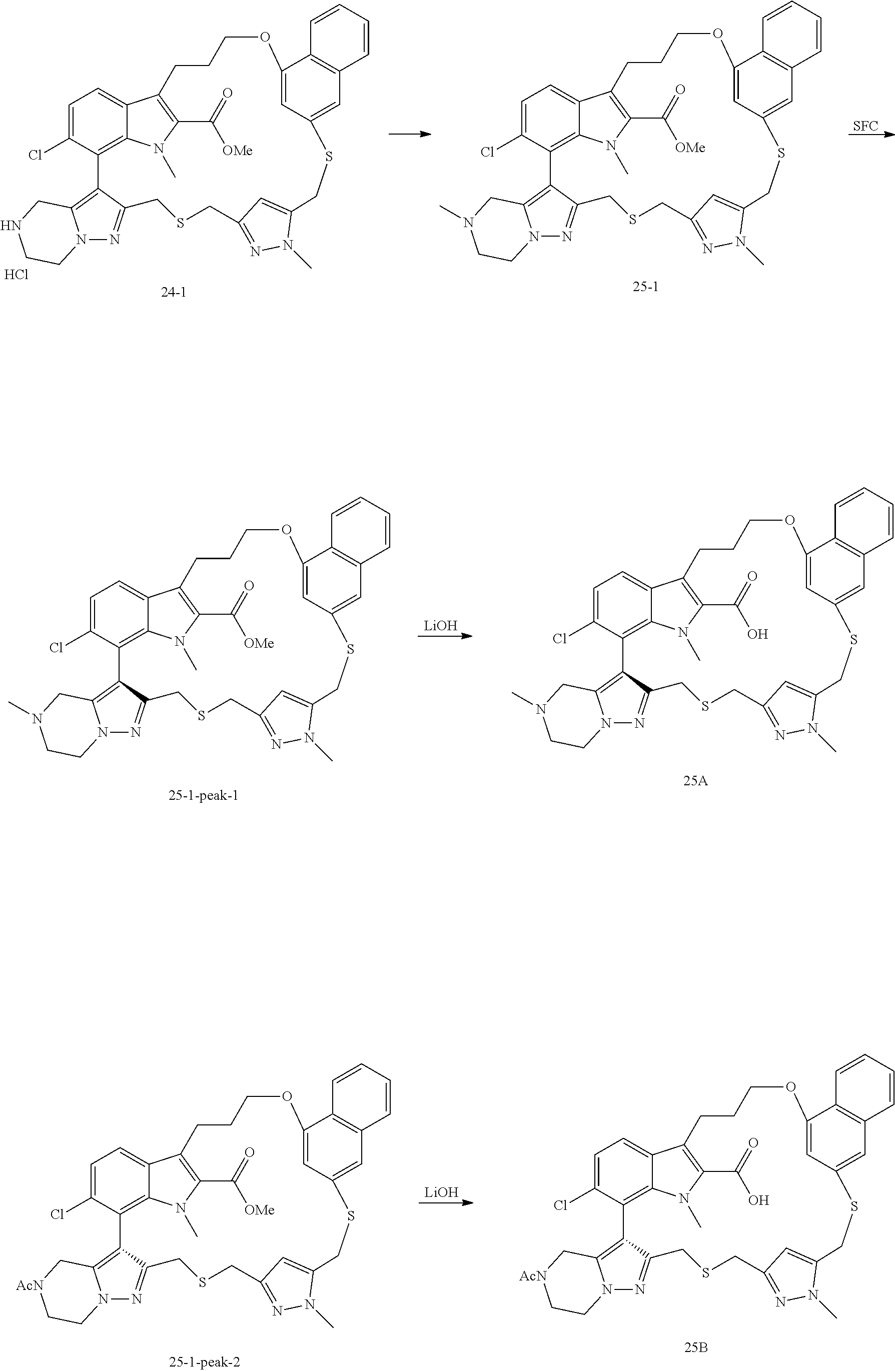

To a stirred solution of methyl (Z)-1⁶-chloro-1¹,6¹-dimethyl-2⁴,2⁵,2⁶,2⁷-tetrahydro-1¹H,6¹H-10-oxa-4,8-dithia-2(3,2)-pyrazolo[1,5-a]pyrazina-1(7,3)-indola-6(3,5)-pyrazola-9(3,1)-naphthalenacyclotridecaphane-1²-carboxylate as the HCl salt (24-1) (280 mg, 0.374 mmol) in MeOH (1.2 mL) were added THF (0.2 mL), TEA (0.26 mL, 1.871 mmol), paraformaldehyde (90 mg, 2.994 mmol) and NaCNBH₃ (118 mg, 1.871 mmol) at rt, and the mixture was stirred at rt for 16 h. The volatiles was evaporated. The reaction was quenched with water (20 mL) and extracted with 3% MeOH/DCM (2×20 mL). The organic layer was separated, dried over Na₂SO₄, filtered and evaporated to get methyl (Z)-1⁶-chloro-1¹2⁵,6¹-trimethyl-2⁴,2⁵,2⁶,2⁷-tetrahydro-1¹H,6¹H-10-oxa-4,8-dithia-2(3,2)-pyrazolo[1,5-a]pyrazina-1(7,3)-indola-6(3,5)-pyrazola-9(3,1)-naphthalenacyclotridecaphane-1²-carboxylate (25-1, 150 mg, 0.206 mmol, 55%) a white solid that was submitted for SFC for separation of enantiomers. MS (LCMS) m/z 727.19 [M+H]⁺. ¹H NMR (400 MHz, CDCl₃) δ 8.32-8.30 (m, 1H), 7.74-7.71 (m, 1H), 7.53-7.50 (m, 4H), 6.93 (d, J=8.4 Hz, 1H), 6.24 (d, J=1.6 Hz, 1H), 4.90 (s, 1H), 4.25-4.13 (m, 2H), 3.89-3.79 (m, 6H), 3.73-3.64 (m, 8H), 3.50-3.46 (m, 2H), 3.34-3.26 (m, 4H), 3.06 (d, J=13.6 Hz, 1H), 2.91-2.89 (m, 2H), 2.65 (d, J=14 Hz, 1H), 2.51-2.40 (m, 2H), 2.24-2.15 (m, 2H).

Methyl (Z)-1⁶-chloro-1¹,2⁵,6¹-trimethyl-2⁴,2⁵,2⁶,2⁷-tetrahydro-1¹H,6¹H-10-oxa-4,8-dithia-2(3,2)-pyrazolo[1,5-a]pyrazina-1(7,3)-indola-6(3,5)-pyrazola-9(3,1)-naphthalenacyclotridecaphane-1²-carboxylate (25-1, 150 mg, LCMS 90%) was purified by SFC purification to afford 25-1-peak-1(45 mg) as an off white solid with 98% of LCMS purity (chiral HPLC:99%) and 25-peak-2 (53 mg) as an off white solid with 97% of LCMS purity (chiral HPLC:99%). These two peaks were separately used for the next steps to get respective final compound.

25-1-peak-1: MS (LCMS) m/z 727.30 [M+H]⁺.

25-1-peak-2: MS (LCMS) m/z 727.19 [M+H]⁺.

To a stirred solution of methyl (Z)-1⁶-chloro-1¹,2⁵,6¹-trimethyl-2⁴ 2⁵,2⁶,2⁷-tetrahydro-1¹H,6¹H-10-oxa-4,8-dithia-2(3,2)-pyrazolo[1,5-a]pyrazina-1(7,3)-indola-6(3,5)-pyrazola-9(3,1)-naphthalenacyclotridecaphane-1²-carboxylate (25-1-peak-1, 45 mg, 0.0619 mmol) in MeOH:THF:H₂O (10:10:3, 5 mL) was added LiOH.H₂O (26 mg, 0.619 mmol) at 0° C., and the mixture stirred at rt for 2 h. Another portion of LiOH.H₂O (13 mg, 0.309 mmol) was added at 0° C., and the mixture was stirred at rt for 1 h. The solvent was evaporated, and the mixture was diluted with water (2 mL). The aqueous layer was acidified to pH 2 using 1N aq. HCl, and the mixture was stirred for 1 min. A solid precipitation was observed. The mixture was filtered, washed with water and dried to give (Z)-1⁶-chloro-1¹,2⁵,6¹-trimethyl-2⁴,2⁵,2⁶,2⁷-tetrahydro-1¹H,6¹H-10-oxa-4,8-dithia-2(3,2)-pyrazolo[1,5-a]pyrazina-1(7,3)-indola-6(3,5)-pyrazola-9(3,1)-naphthalenacyclotridecaphane-1²-carboxylic acid (25A, 38.8 mg, 0.054 mmol, 88%) as an off white solid. MS (LCMS) m/z 713.32 [M+H]⁺. ¹H NMR (400 MHz, DMSO-d₆) δ 13.4 (br s, 1H), 8.10 (d, J=7.6 Hz, 1H), 7.89 (br s, 1H), 7.73 (d, J=8.8 Hz, 1H), 7.51-7.44 (m, 2H), 7.39 (m, 1H), 7.13 (br s, 1H), 6.62 (s, 1H), 4.76 (br s, 1H), 4.63-4.01 (m, 7H), 3.69 (br s, 2H), 3.69 (s, 3H), 3.58 (s, 3H), 3.47-3.43 (m, 3H), 3.23 (d, J=13.2 Hz, 1H), 3.11-3.09 (m, 2H), 2.89 (d, J=14 Hz, 4H), 3.36-3.32 (m, 1H), 3.32-3.21 (m, 1H). HPLC: 96.12%, Chiral purity: 98.94%.

To a stirred solution of methyl (Z)-1⁶-chloro-1¹,2⁵,6¹-trimethyl-2⁴,2⁵,2⁶,2⁷-tetrahydro-1¹H,6¹H-10-oxa-4,8-dithia-2(3,2)-pyrazolo[1,5-a]pyrazina-1(7,3)-indola-6(3,5)-pyrazola-9(3,1)-naphthalenacyclotridecaphane-1²-carboxylate (25-1-peak-2, 50 mg, 0.0688 mmol) in MeOH:THF:H₂O (10:10:3, 5 mL) was added LiOH.H₂O (29 mg, 0.688 mmol) at 0° C., and the mixture was stirred at rt for 2 h. Another portion of LiOH.H₂O (15 mg, 0.344 mmol) was added at 0° C., and the mixture was stirred at rt for 1 h. The solvent was evaporated, and the mixture was diluted with water (2 mL). The aqueous layer was acidified to pH 2 using 1N aq. HCl, and the mixture was stirred for 1 min. A solid precipitation was observed. The mixture was filtered, washed with water and dried to give (Z)-1⁶-chloro-1¹,2⁵,6¹-trimethyl-2⁴,2⁵,2⁶,2⁷-tetrahydro-1¹H,6¹H-10-oxa-4,8-dithia-2(3,2)-pyrazolo[1,5-a]pyrazina-1(7,3)-indola-6(3,5)-pyrazola-9(3,1)-naphthalenacyclotridecaphane-1²-carboxylic acid (25B, 32.9 mg, 0.046 mmol, 67%) as an off white solid. MS (LCMS) m/z 713.32 [M+H]⁺. ¹H NMR (400 MHz, DMSO-d₆) δ 13.4 (br s, 1H), 8.09 (d, J=8 Hz, 1H), 7.88 (d, J=8.4 Hz, 1H), 7.72 (d, J=8.8 Hz, 1H), 7.51-7.44 (m, 2H), 7.39 (m, 1H), 7.13 (d, J=8.4 Hz, 1H), 6.65 (s, 1H), 4.74 (s, 1H), 4.25 (s, 2H), 4.13-4.08 (m, 3H), 3.85-3.84 (m, 1H), 3.70 (s, 3H), 3.54 (s, 3H), 3.45 (d, J=12.4 Hz, 2H), 3.32 (s, 3H), 3.21 (d, J=12.8 Hz, 1H), 3.13-3.06 (m, 2H), 2.89 (d, J=14.4 Hz, 2H), 2.35-2.23 (m, 4H). HPLC: 95.50%, Chiral purity: 99.69%.

The absolute stereochemistry of compounds (25A) and (25B) is arbitrarily assigned.

### Example 26

### (Z)-1⁶-Chloro-1¹,6¹-dimethyl-2⁵,2⁶-dihydro-1¹H,2⁴H,6¹H-10-oxa-8-thia-4-aza-1(7,3)-indola-2(3,2)-pyrrolo[1,2-b]pyrazola-6(3,5)-pyrazola-9(3,1)-naphthalenacyclotridecaphane-1²-carboxylic acid

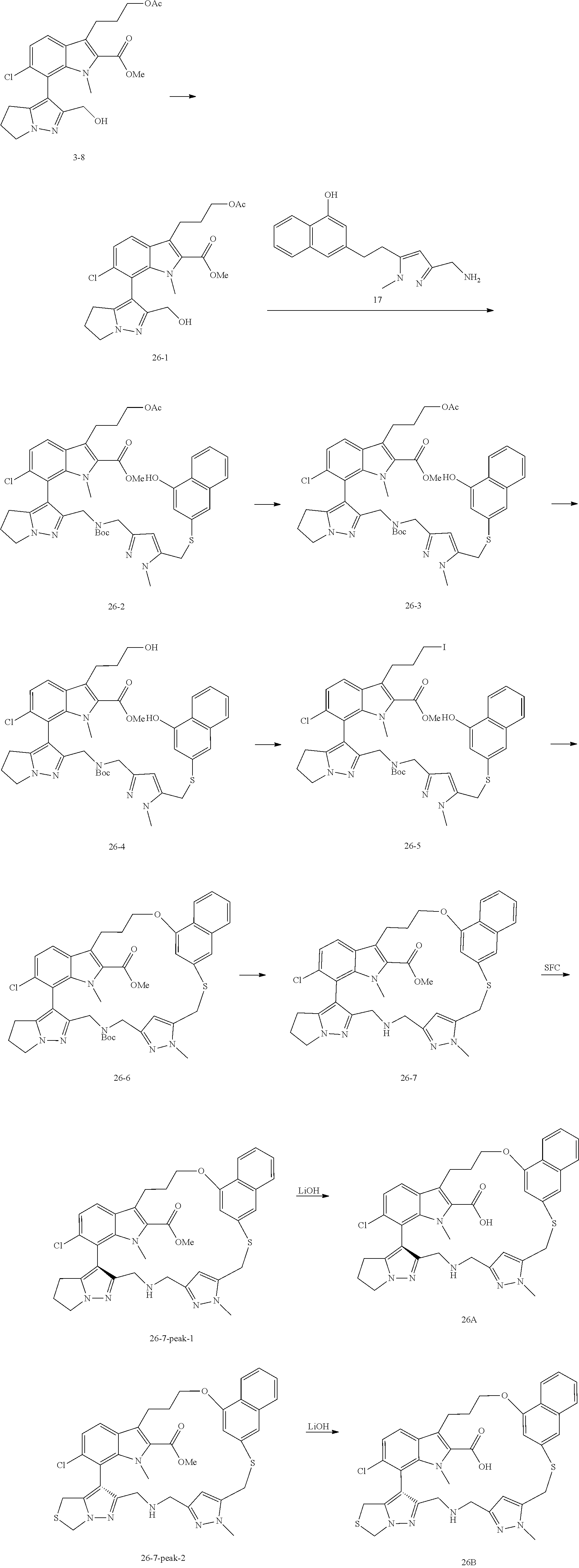

To a stirred solution of methyl 3-(3-acetoxypropyl)-6-chloro-7-(2-(hydroxymethyl)-5,6-dihydro-4H-pyrrolo[1,2-b]pyrazol-3-yl)-1-methyl-1H-indole-2-carboxylate (3-8, 6.6 g, 14.37 mmol) in CH₂Cl₂ (70 mL) was added Dess-Martin periodinane (6.702 g, 15.80 mmol) and NaHCO₃ (5.431 g, 64.66 mmol) at 0° C., and the mixture was allowed to warmed to rt. The mixture was stirred for 1 h. The mixture was diluted with DCM (200 mL) and washed with water (100 mL) and brine (100 mL), dried over anhydrous Na₂SO₄, filtered and concentrated under reduced pressure. The crude was purified by silica gel column to afford methyl 3-(3-acetoxypropyl)-6-chloro-7-(2-formyl-5,6-dihydro-4H-pyrrolo[1,2-b]pyrazol-3-yl)-1-methyl-1H-indole-2-carboxylate (26-1, 4.5 g, 9.85 mmol, 68%) as a light yellow gummy liquid. MS (ESI) m/z 458.31 [M+H]⁺. ¹H NMR (400 MHz, DMSO-d₆) δ 9.82 (s, 1H), 7.74 (d, J=8.4 Hz, 1H), 7.26 (d, J=8.4 Hz, 1H), 4.34 (t, J=7.2 Hz, 2H), 4.03 (t, J=6.4 Hz, 2H), 3.85 (s, 3H), 3.38 (s, 3H), 3.04 (t, J=7.2 Hz, 2H), 2.82-2.70 (m, 2H), 2.70-2.55 (m, 2H), 2.01 (s, 3H), 1.95-1.85 (m, 2H).

To a stirred solution of methyl 3-(3-acetoxypropyl)-6-chloro-7-(2-formyl-5,6-dihydro-4H-pyrrolo[1,2-b]pyrazol-3-yl)-1-methyl-1H-indole-2-carboxylate (26-1, 6 g, 13.12 mmol) in 1,2-DCE (90 mL) was added 3-(2-(3-(aminomethyl)-1-methyl-1H-pyrazol-5-yl)ethyl)naphthalen-1-ol (17, 3.686 g, 13.12 mmol). The mixture was stirred for 10 mins, and then sodium triacetoxy borohydride (4.172 g, 19.68 mmol) was added portionwise at 0° C. The mixture was allowed to warm to rt and then stirred for 16 h. The mixture was diluted with DCM (200 mL) and washed with water (100 mL) and brine (100 mL), dried over anhydrous Na₂SO₄, filtered and concentrated under reduced pressure. The crude was purified by silica gel column and eluted at 5% MeOH in DCM to afford methyl 3-(3-acetoxypropyl)-6-chloro-7-(2-((((5-(2-(4-hydroxynaphthalen-2-yl)ethyl)-1-methyl-1H-pyrazol-3-yl)methyl)amino)methyl)-5,6-dihydro-4H-pyrrolo[1,2-b]pyrazol-3-yl)-1-methyl-1H-indole-2-carboxylate (26-2, 4.5 g, 6.23 mmol, 47%) as a grey colored solid. MS (ESI) m/z 723.58 [M+H]⁺. ¹H NMR (400 MHz, DMSO-d₆) δ 10.04 (s, 1H), 8.06 (d, J=8.4 Hz, 1H), 7.75-7.65 (m, 2H), 7.45-7.35 (m, 2H), 7.28 (d, J=8.8 Hz, 1H), 7.14 (s, 1H), 6.77 (s, 1H), 5.84 (br s, 1H), 4.25-4.15 (m, 2H), 4.00 (t, J=6.4 Hz, 2H), 3.84 (s, 3H), 3.75-3.60 (m, 3H), 3.56 (s, 3H), 3.47 (s, 3H), 3.10-2.90 (m, 3H), 2.86 (s, 3H), 2.75-2.50 (m, 4H), 1.97 (s, 3H), 1.95-1.80 (m, 2H).

To a stirred solution of methyl 3-(3-acetoxypropyl)-6-chloro-7-(2-((((5-(2-(4-hydroxynaphthalen-2-yl)ethyl)-1-methyl-1H-pyrazol-3-yl)methyl)amino)methyl)-5,6-dihydro-4H-pyrrolo[1,2-b]pyrazol-3-yl)-1-methyl-1H-indole-2-carboxylate (26-2, 4.2 g, 5.817 mmol) in DCM (50 mL) was added Boc-anhydride (2.536 g, 11.63 mmol) at rt, and the mixture was stirred for 16 h. The mixture was diluted with DCM (100 mL) and washed with water (100 mL) and brine (100 mL), dried over anhydrous Na₂SO₄, filtered and concentrated under reduced pressure. The crude was purified by silica gel column and eluted at 30% EtOAc in PE to afford methyl 3-(3-acetoxypropyl)-7-(2-(((tert-butoxycarbonyl)((5-(2-(4-hydroxynaphthalen-2-yl)ethyl)-1-methyl-1H-pyrazol-3-yl)methyl)amino)methyl)-5,6-dihydro-4H-pyrrolo[1,2-b]pyrazol-3-yl)-6-chloro-1-methyl-1H-indole-2-carboxylate (26-3, 3.0 g, 3.65 mmol, 62%) as a pale yellow solid. MS (ESI) m/z 823.64 [M+H]⁺. ¹H NMR (400 MHz, CDCl₃) δ 9.15-9.0 (m, 1H), 8.20 (d, J=8.0 Hz, 1H), 7.71 (d, J=8.0 Hz, 1H), 7.55-7.50 (m, 1H), 7.45-7.35 (m, 2H), 7.26-7.15 (m, 2H), 6.10-6.00 (m, 1H), 5.88 (s, 1H), 4.70-4.40 (m, 3H), 4.40-4.25 (m, 3H), 4.14 (t, J=6.4 Hz, 3H), 3.92 (s, 3H), 3.65-3.55 (m, 3H), 3.15-3.05 (m, 3H), 3.00-2.60 (m, 8H), 2.10 (s, 3H), 2.05-1.95 (m, 2H), 1.20-0.90 (m, 9H).

To a stirred solution of methyl 3-(3-acetoxypropyl)-7-(2-(((tert-butoxycarbonyl)((5-(2-(4-hydroxynaphthalen-2-yl)ethyl)-1-methyl-1H-pyrazol-3-yl)methyl)amino)methyl)-5,6-dihydro-4H-pyrrolo[1,2-b]pyrazol-3-yl)-6-chloro-1-methyl-1H-indole-2-carboxylate (26-3, 1.5 g, 1.824 mmol) in MeOH (25 mL) was added NaHCO₃ (1.072 g, 12.76 mmol), and the mixture was heated at 60° C. for 3 h. The mixture was diluted with DCM (200 mL), filtered through a Celite pad. The pad was washed with DCM (100 mL). The filtrate was concentrated under reduced pressure to afford methyl 7-(2-(((tert-butoxycarbonyl)((5-(2-(4-hydroxynaphthalen-2-yl)ethyl)-1-methyl-1H-pyrazol-3-yl)methyl)amino)methyl)-5,6-dihydro-4H-pyrrolo[1,2-b]pyrazol-3-yl)-6-chloro-3-(3-hydroxypropyl)-1-methyl-1H-indole-2-carboxylate (26-4, 1.5 g, 1.92 mmol, crude) as a pink colored solid. The crude compound used without further purification. MS (ESI) m/z 781.58 [M+H]⁺.

To a stirred solution methyl 7-(2-(((tert-butoxycarbonyl)((5-(2-(4-hydroxynaphthalen-2-yl)ethyl)-1-methyl-1H-pyrazol-3-yl)methyl)amino)methyl)-5,6-dihydro-4H-pyrrolo[1,2-b]pyrazol-3-yl)-6-chloro-3-(3-hydroxypropyl)-1-methyl-1H-indole-2-carboxylate (26-4, 1.5 g, 1.923 mmol) in DMF (20 mL) was added methyltriphenoxyphosphonium iodide (2.607 g, 5.769 mmol) at 0° C., and the mixture was at the same temperature for 20 min. The mixture was diluted with EtOAc (50 mL), washed with water (3×50 mL) and brine (2×50 mL), dried over anhydrous Na₂SO₄, filtered and concentrated under reduced pressure. The crude was purified by silica gel column and eluted at 30% to 40% EtOAc in PE to afford methyl 7-(2-(((tert-butoxycarbonyl)((5-(2-(4-hydroxynaphthalen-2-yl)ethyl)-1-methyl-1H-pyrazol-3-yl)methyl)amino)methyl)-5,6-dihydro-4H-pyrrolo[1,2-b]pyrazol-3-yl)-6-chloro-3-(3-iodopropyl)-1-methyl-1H-indole-2-carboxylate (26-5, 1.35 g, 1.52 mmol, 615 over 2-steps) as a pale yellow solid. MS (ESI) m/z 891.63 [M+H]⁺.

To a stirred solution of methyl 7-(2-(((tert-butoxycarbonyl)((5-(2-(4-hydroxynaphthalen-2-yl)ethyl)-1-methyl-1H-pyrazol-3-yl)methyl)amino)methyl)-5,6-dihydro-4H-pyrrolo[1,2-b]pyrazol-3-yl)-6-chloro-3-(3-iodopropyl)-1-methyl-1H-indole-2-carboxylate (26-5, 1.35 g, 1.516 mmol) in acetonitrile (420 mL) was added potassium carbonate (0.627 g, 4.548 mmol), and the mixture was heated at 60° C. for 16 h. The mixture was filtered and concentrated under reduced pressure. The compound was purified by silica gel column and eluted at 30-40% EtOAc in PE to afford to afford 4-(tert-butyl) 1²-methyl (Z)-1⁶-chloro-1¹,6¹-dimethyl-2⁵,2⁶-dihydro-1¹H,2⁴H,6¹H-10-oxa-4-aza-1(7,3)-indola-2(3,2)-pyrrolo[1,2-b]pyrazola-6(3,5)-pyrazola-9(3,1)-naphthalenacyclotridecaphane-1²,4 dicarboxylate (26-6, 0.470 g, 0.62 mmol, 40%) as an off-white solid. MS (ESI) m/z 763.52 [M+H]⁺.

To a stirred solution of 4-(tert-butyl) 1²-methyl (Z)-1⁶-chloro-1¹,6¹-dimethyl-2⁵,2⁶-dihydro-1¹H,2⁴H,6¹H-10-oxa-4-aza-1(7,3)-indola-2(3,2)-pyrrolo[1,2-b]pyrazola-6(3,5)-pyrazola-9(3,1)-naphthalenacyclotridecaphane-1²,4-dicarboxylate (26-6, 470 mg, 0.616 mmol) in 1,4-dioxane (4 mL) was added triethylsilane (286 mg, 2.467 mmol) and 4M HCl in 1,4-dioxane (4 mL) at 0° C. The mixture was allowed to warmed to rt and then stirred for 1 h. The mixture was concentrated under reduced pressure, and the residue was dissolved in 10% MeOH in DCM (50 mL). The mixture was washed with a sat. aq. NaHCO₃ solution (20 mL), water (50 mL) and brine (50 mL). The organic layer was dried over anhydrous Na₂SO₄, filtered and concentrated under reduced pressure to afford methyl (Z)-1⁶-chloro-1¹,6¹-dimethyl-2⁵,2⁶-dihydro-1¹H,2⁴H,6¹H-10-oxa-4-aza-1(7,3)-indola-2(3,2)-pyrrolo[1,2-b]pyrazola-6(3,5)-pyrazola-9(3,1)-naphthalenacyclotridecaphane-1²-carboxylate (26-7, 340 mg, 0.513 mmol, 83%) as an off-white solid. MS (ESI) m/z 663.55 [M+H]⁺. ¹H NMR (400 MHz, DMSO-d₆) δ 8.07 (d, J=8.0 Hz, 1H), 7.79 (d, J=8.4 Hz, 1H), 7.70 (d, J=7.6 Hz, 1H), 7.46-7.36 (m, 2H), 7.20 (s, 1H), 7.13 (d, J=8.4 Hz, 1H), 6.43 (s, 1H), 4.88 (s, 1H), 4.12 (d, J=6.8 Hz, 1H), 4.00-3.95 (m, 1H), 3.82 (s, 3H), 3.80-3.70 (m, 1H), 3.57 (s, 3H), 3.53 (s, 3H), 3.50 (s, 2H), 3.45-3.35 (m, 3H), 3.15-3.10 (m, 3H), 3.05-2.95 (m, 3H), 2.70-2.60 (m, 2H), 2.60-2.50 (m, 2H), 2.35-2.10 (m, 3H),

Racemic methyl (Z)-1⁶-chloro-1¹,6¹-dimethyl-2⁵,2⁶-dihydro-1¹H,2⁴H,6¹H-10-oxa-4-aza-1(7,3)-indola-2(3,2)-pyrrolo[1,2-b]pyrazola-6(3,5)-pyrazola-9(3,1)-naphthalenacyclotridecaphane-1²-carboxylate (26-7, 340 mg) was purified by chiral SFC purification and separated 26-7-peak-1 (140 mg) and 26-7-peak-2 (130 mg) as an off-white solid.

26-7-peak-1: MS (LCMS) m/z 663.68 [M+H]⁺. LCMS purity: 95.93%; Chiral HPLC purity: 99.90%.

26-7-peak-1: MS (LCMS) m/z 663.47 [M+H]⁺. LCMS purity: 98.18%; Chiral HPLC purity: 98.88%.

To a stirred solution of methyl (Z)-1⁶-chloro-1¹,6¹-dimethyl-2⁵,2⁶-dihydro-1¹H,2⁴H,6¹H-10-oxa-4-aza-1(7,3)-indola-2(3,2)-pyrrolo[1,2-b]pyrazola-6(3,5)-pyrazola-9(3,1)-naphthalenacyclotridecaphane-1²-carboxylate (26-7-peak-1, 130 mg, 0.196 mmol) in MeOH:THF:H₂O (1:1:1, 6 mL) was added LiOH.H₂O (123 mg, 2.945 mmol), and the mixture was heated at 60° C. for 1 h. The solvent was evaporated, and the aqueous layer was acidified to pH 2 using 2 N aq. HCl. The solid was filtered off, washed with water (5 mL), dried under vacuum to afford (Z)-1⁶-chloro-1¹,6¹-dimethyl-2⁵,2⁶-dihydro-1¹H,2⁴H,6¹H-10-oxa-4-aza-1(7,3)-indola-2(3,2)-pyrrolo[1,2-b]pyrazola-6(3,5)-pyrazola-9(3,1)-naphthalenacyclotridecaphane-1²-carboxylic acid (26A, 100 mg, 78%, 0.154 mmol) as an off-white solid. MS (ESI) m/z 649.48 [M+H]⁺. ¹H NMR (400 MHz, DMSO-d₆) δ 13.40 (br s, 1H), 9.30 (br s, 1H), 8.90 (br s, 1H), 8.08 (d, J=8.0 Hz, 1H), 7.81 (d, J=8.8 Hz, 1H), 7.50-7.40 (m, 2H), 7.72 (d, J=7.6 Hz, 1H), 7.22 (s, 1H), 7.10 (d, J=8.8 Hz, 1H), 6.29 (s, 1H), 5.20 (s, 1H), 4.22 (t, J=7.4 Hz, 2H), 3.90-3.82 (m, 1H), 3.80-3.72 (m, 2H), 3.70-3.58 (m, 2H), 3.55 (s, 3H), 3.50-3.40 (m, 5H), 3.15-3.05 (m, 2H), 2.90-2.55 (m, 6H), 2.40-2.30 (m, 1H), 2.30-2.20 (m, 1H). LCMS purity: 96.76%; HPLC purity: 96.68%; Chiral HPLC purity: 99.32%.

To a stirred solution of methyl (Z)-1⁶-chloro-1¹,6¹-dimethyl-2⁵,2⁶-dihydro-1¹H,2⁴H,6¹H-10-oxa-4-aza-1(7,3)-indola-2(3,2)-pyrrolo[1,2-b]pyrazola-6(3,5)-pyrazola-9(3,1)-naphthalenacyclotridecaphane-1²-carboxylate (26-7-peak-2, 130 mg, 0.196 mmol) in MeOH:THF:H₂O (1:1:1, 6 mL) was added LiOH.H₂O (123 mg, 2.945 mmol), and the mixture was heated at 60° C. for 1 h. The solvent was evaporated, and the aqueous layer was acidified to pH 2 using 2 N aq. HCl. The solid was filtered off, washed with water (5 mL), dried under vacuum to afford (Z)-1⁶-chloro-1¹,6¹-dimethyl-2⁵,2⁶-dihydro-1¹H,2⁴H,6¹H-10-oxa-4-aza-1(7,3)-indola-2(3,2)-pyrrolo[1,2-b]pyrazola-6(3,5)-pyrazola-9(3,1)-naphthalenacyclotridecaphane-1²-carboxylic acid (26B, 105 mg) as an off-white solid. MS (ESI) m/z 649.48 [M+H]⁺. ¹H NMR (400 MHz, DMSO-d₆) δ 13.40 (br s, 1H), 9.30 (br s, 1H), 8.90 (br s, 1H), 8.08 (d, J=8.0 Hz, 1H), 7.72 (d, J=7.6 Hz, 1H), 7.81 (d, J=8.8 Hz, 1H), 7.50-7.40 (m, 2H), 7.22 (s, 1H), 7.10 (d, J=8.8 Hz, 1H), 6.29 (s, 1H), 5.20 (s, 1H), 4.22 (t, J=7.4 Hz, 2H), 3.90-3.82 (m, 1H), 3.80-3.72 (m, 2H), 3.70-3.58 (m, 2H), 3.55 (s, 3H), 3.50-3.40 (m, 5H), 3.15-3.05 (m, 2H), 2.90-2.55 (m, 6H), 2.40-2.30 (m, 1H), 2.30-2.20 (m, 1H). LCMS purity: 99.27%; HPLC purity: 98.43%; Chiral HPLC purity: 98.24%.

The absolute stereochemistry of compounds (26A) and (26B) is arbitrarily assigned.

### Example 27

### (Z)-1⁶-Chloro-1¹,6¹-dimethyl-2⁴,2⁵,2⁶,2⁷-tetrahydro-1¹H,6¹H-10-oxa-4-aza-2(3,2)-pyrazolo[1,5-a]pyridina-1(7,3)-indola-6(3,5)-pyrazola-9(3,1)-naphthalenacyclotridecaphane-1²-carboxylic acid

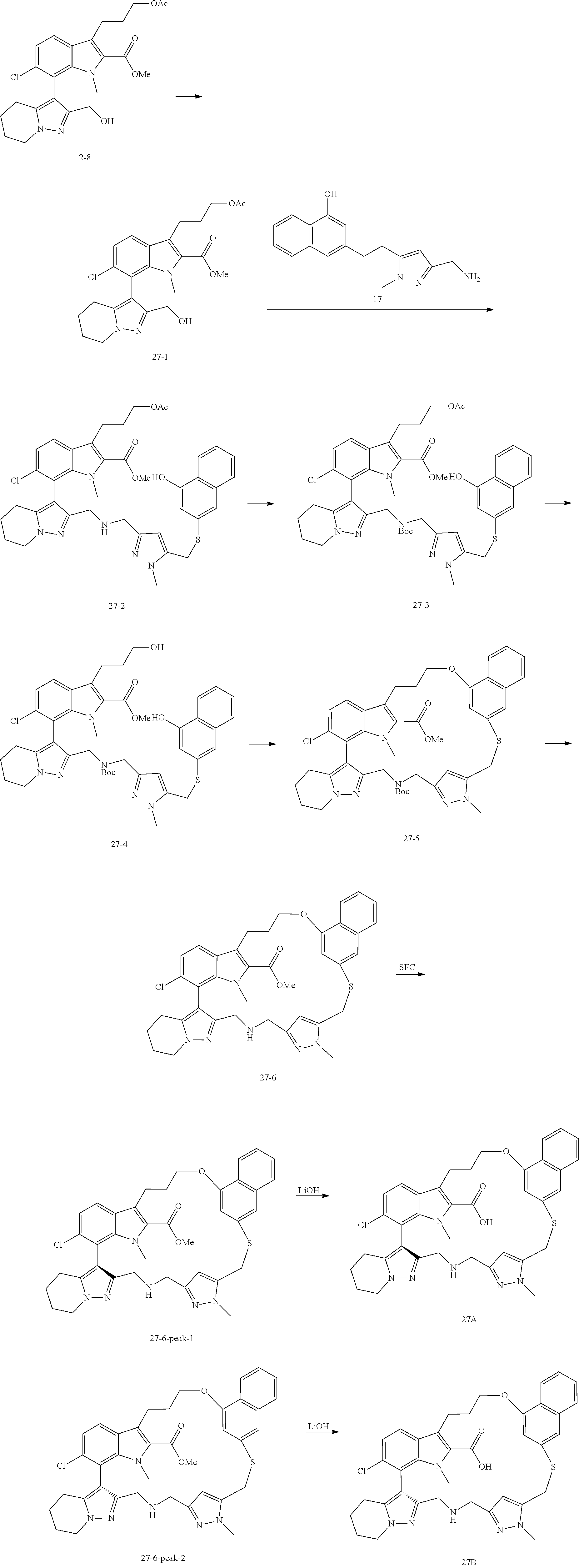

To a stirred solution of methyl 3-(3-acetoxypropyl)-6-chloro-7-(2-(hydroxymethyl)-4,5,6,7-tetrahydropyrazolo[1,5-a]pyridin-3-yl)-1-methyl-1H-indole-2-carboxylate (2-8, 2.5 g, 5.27 mmol) in CH₂Cl₂ (25 mL) was added Dess-Martin periodinane (2.68 g, 6.3 mmol) and NaHCO₃ (2.62 g, 3.12 mmol) at 0° C. The mixture was allowed to warm at rt and then stirred for 1 h. The mixture was diluted with DCM (100 mL), washed with water (100 mL) and brine (100 mL), dried over anhydrous Na₂SO₄, filtered and concentrated under reduced pressure. The crude was purified by silica gel column to afford methyl 3-(3-acetoxypropyl)-6-chloro-7-(2-formyl-4,5,6,7-tetrahydropyrazolo[1,5-a]pyridin-3-yl)-1-methyl-1H-indole-2-carboxylate (27-1, 2.2 g, 4.6 mmol, 67%) as a light yellow gummy liquid. MS (ESI) m/z 472.34 [M+H]⁺.

To a stirred solution of methyl 3-(3-acetoxypropyl)-6-chloro-7-(2-formyl-4,5,6,7-tetrahydropyrazolo[1,5-a]pyridin-3-yl)-1-methyl-1H-indole-2-carboxylate (27-1, 500 mg, 1.06 mmol) in methanol (10 mL) were added 3-(2-(3-(aminomethyl)-1-methyl-1H-pyrazol-5-yl)ethyl)naphthalen-1-ol (17) (298 mg, 1.06 mmol), 4 A° molecular sieves (200 mg) and triethyl amine (0.2 mL, 1.59 mmol). The mixture was stirred for 16 h at 65° C., cooled to rt. Sodium borohydride (80 mg, 212 mmol) was added portionwise at 0° C. The mixture was allowed to warm to rt and then stirred for 1 h. The mixture was diluted with DCM (20 mL), washed with water (10 mL) and brine (10 mL), dried over anhydrous Na₂SO₄, filtered and concentrated under reduced pressure. The crude was purified by silica gel column and eluted at 5% MeOH in DCM to afford methyl 3-(3-acetoxypropyl)-6-chloro-7-(2-((((5-(2-(4-hydroxynaphthalen-2-yl)ethyl)-1-methyl-1H-pyrazol-3-yl)methyl)amino)methyl)-4,5,6,7-tetrahydropyrazolo[1,5-a]pyridin-3-yl)-1-methyl-1H-indole-2-carboxylate (27-2, 250 mg, 0.339 mmol, 32%) as an off white solid. MS (ESI) m/z 737.72 [M+H]⁺. ¹H NMR (400 MHz, CDCl₃) δ 8.20 (d, J=8.4 Hz, 1H), 7.71 (d, J=7.6 Hz, 1H), 7.57 (d, J=8.8 Hz, 1H), 7.43-7.38 (m, 2H), 7.25 (d, J=4.8 Hz, 1H), 7.20 (s, 1H), 6.31 (s, 1H), 6.03 (s, 1H), 4.27 (t, J=6 Hz, 2H), 4.20 (t, J=6.4 Hz, 3H), 3.91 (s, 3H), 3.79-3.60 (m, 4H), 3.56 (s, 3H), 3.20 (s, 3H), 3.12-3.08 (m, 2H), 2.97-2.88 (m, 4H), 2.53-2.45 (m, 2H), 2.09-1.98 (m, 5H), 1.84 (s, 2H).

To a stirred solution of methyl 3-(3-acetoxypropyl)-6-chloro-7-(2-((((5-(2-(4-hydroxynaphthalen-2-yl)ethyl)-1-methyl-1H-pyrazol-3-yl)methyl)amino)methyl)-4,5,6,7-tetrahydropyrazolo[1,5-a]pyridin-3-yl)-1-methyl-1H-indole-2-carboxylate (27-2, 500 mg, 0.678 mmol) in DCM (10 mL) was added Boc-anhydride (0.2 g, 0.814 mmol) at rt, and the mixture was stirred for 16 h. The mixture was diluted with DCM (20 mL), washed with water (10 mL) and brine (10 mL), dried over anhydrous Na₂SO₄, filtered and concentrated under reduced pressure. The crude was purified by silica gel column and eluted at 0.2% Methanol in DCM to afford methyl 3-(3-acetoxypropyl)-7-(2-(((tert-butoxycarbonyl)((5-(2-(4-hydroxynaphthalen-2-yl)ethyl)-1-methyl-1H-pyrazol-3-yl)methyl)amino)methyl)-4,5,6,7-tetrahydropyrazolo[1,5-a]pyridin-3-yl)-6-chloro-1-methyl-1H-indole-2-carboxylate (27-3, 450 mg, 0.538 mmol, 83%) as a pale yellow solid. MS (ESI) m/z 835.63 [M+H]⁺. ¹H NMR (400 MHz, CDCl₃) δ 9.1-9.0 (m, 1H), 8.20 (d, J=8.0 Hz, 1H), 7.71 (d, J=7.6 Hz, 1H), 7.55-7.50 (m, 1H), 7.43-7.39 (m, 2H), 7.12 (s, 1H), 6.05-6.00 (m, 1H), 5.90 (s, 1H), 4.58-4.46 (m, 3H), 4.32-4.29 (m, 3H), 4.14 (t, J=6.4 Hz, 3H), 3.92 (s, 3H), 3.58-3.56 (m, 3H), 3.1-3.06 (m, 5H), 2.96-2.90 (m, 3H), 2.49-2.42 (m, 2H), 2.17-1.8 (m, 9H), 1.14-0.97 (m, 9H).

To a stirred solution of methyl 3-(3-acetoxypropyl)-7-(2-(((tert-butoxycarbonyl)((5-(2-(4-hydroxynaphthalen-2-yl)ethyl)-1-methyl-1H-pyrazol-3-yl)methyl)amino)methyl)-4,5,6,7-tetrahydropyrazolo[1,5-a]pyridin-3-yl)-6-chloro-1-methyl-1H-indole-2-carboxylate (27-3, 750 mg, 0.897 mmol) in MeOH (10 mL) was added NaHCO₃ (452 mg, 5.38 mmol), and the mixture was heated at 60° C. for 3 h. The mixture was diluted with DCM (50 mL) and filtered through a Celite pad. The pad was washed with DCM (10 mL). The filtrate was concentrated under reduced pressure to afford methyl 7-(2-(((tert-butoxycarbonyl)((5-(2-(4-hydroxynaphthalen-2-yl)ethyl)-1-methyl-1H-pyrazol-3-yl)methyl)amino)methyl)-4,5,6,7-tetrahydropyrazolo[1,5-a]pyridin-3-yl)-6-chloro-3-(3-hydroxypropyl)-1-methyl-1H-indole-2-carboxylate (27-4, 600 mg, 0.755 mmol, 84%) as an off white solid. MS (ESI) m/z 793.10 [M+H]⁺.

To a stirred solution of TPP (528 mg, 2.01 mmol) in toluene (10 mL) was added a solution of di-tert-butyl diazene-1,2-dicarboxylate (462 mg, 2.01 mmol) and methyl 7-(2-(((tert-butoxycarbonyl)((5-(2-(4-hydroxynaphthalen-2-yl)ethyl)-1-methyl-1H-pyrazol-3-yl)methyl)amino)methyl)-4,5,6,7-tetrahydropyrazolo[1,5-a]pyridin-3-yl)-6-chloro-3-(3-hydroxypropyl)-1-methyl-1H-indole-2-carboxylate (27-4, 400 mg, 0.5 mmol) in toluene (5 mL) and THF (2 mL) at 90° C., and the mixture was stirred at 90° C. for 4 h. The reaction mixture was quenched with water (20 mL) and extracted with EtOAc (2×50 mL). The organic layer was dried over Na₂SO₄, filtered and concentrated to give the semi pure compound that was purified by silica gel column chromatography using 50% EtOAc in PE to afford 27-5. The compound was submitted for prep HPLC for further purification to afford 4-(tert-butyl) 1²-methyl (Z)-1⁶-chloro-1¹,6¹-dimethyl-2⁴,2⁵,2⁶,2⁷-tetrahydro-1¹H,6¹H-10-oxa-4-aza-2(3,2)-pyrazolo[1,5-a]pyridina-1(7,3)-indola-6(3,5)-pyrazola-9(3,1)-naphthalenacyclotridecaphane-12,4-dicarboxylate (27-5, 60 mg, 0.07 mmol, 15%) as a pale yellow solid. MS (ESI) m/z 777.65 [M+H]⁺.

To a stirred solution of 4-(tert-butyl) 1²-methyl (Z)-1⁶-chloro-1¹,6¹-dimethyl-2⁴,2⁵,2⁶,2⁷-tetrahydro-1¹H,6¹H-10-oxa-4-aza-2(3,2)-pyrazolo[1,5-a]pyridina-1(7,3)-indola-6(3,5)-pyrazola-9(3,1)-naphthalenacyclotridecaphane-12,4-dicarboxylate (27-5, 100 mg, 0.128 mmol) in 1,4-dioxane (1 mL) were added triethylsilane (0.6 mL, 2.68 mmol) and 4M HCl in 1,4-dioxane (1 mL) at 0° C. The mixture was allowed to warm to rt and then stirred for 1 h. The mixture was concentrated under reduced pressure. The residue was dissolved in 10% MeOH in DCM (10 mL), washed with a sat. aq.NaHCO₃ solution (5 mL), water (5 mL) and brine (5 mL). The organic layer was dried over anhydrous Na₂SO₄, filtered and concentrated under reduced pressure to afford methyl (Z)-1⁶-chloro-1¹,6¹-dimethyl-2⁴,2⁵,2⁶,2⁷-tetrahydro-1¹H,6¹H-10-oxa-4-aza-2(3,2)-pyrazolo[1,5-a]pyridina-1(7,3)-indola-6(3,5)-pyrazola-9(3,1)-naphthalenacyclotridecaphane-12-carboxylate (27-6, 95 mg, 0.14 mmol, 99%) as an off-white solid. MS (ESI) m/z 677.55 [M+H]⁺.

Racemic methyl (Z)-1⁶-chloro-1¹,6¹-dimethyl-2⁴,2⁵,2⁶,2⁷-tetrahydro-1¹H,6¹H-10-oxa-4-aza-2(3,2)-pyrazolo[1,5-a]pyridina-1(7,3)-indola-6(3,5)-pyrazola-9(3,1)-naphthalenacyclotridecaphane-12-carboxylate (27-6, 95 mg) was purified by chiral SFC purification and separated 27-6-peak-1 (30 mg) and 27-6-peak-2 (28 mg) as an off-white solid.

27-6-peak-1: Chiral HPLC purity: 99.96%.

27-6-peak-2: Chiral HPLC purity: 99.20%.

To a stirred solution of methyl (Z)-1⁶-chloro-1¹,6¹-dimethyl-2⁴,2⁵,2⁶,2⁷-tetrahydro-1¹H,6¹H-10-oxa-4-aza-2(3,2)-pyrazolo[1,5-a]pyridina-1(7,3)-indola-6(3,5)-pyrazola-9(3,1)-naphthalenacyclotridecaphane-12-carboxylate (27-6-peak-1, 30 mg, 0.044 mmol) in MeOH:THF:H₂O (1:1:1, 3 mL) was added LiOH.H₂O (28 mg, 0.665 mmol), and the mixture was heated at 60° C. for 1 h. The solvent was evaporated, and the aqueous layer was acidified to pH 2 using 2N aq. HCl. The solid was filtered off, washed with water (5 mL), dried under vacuum to afford (Z)-1⁶-chloro-1¹,6¹-dimethyl-2⁴,2⁵,2⁶,2⁷-tetrahydro-1¹H,6¹H-10-oxa-4-aza-2(3,2)-pyrazolo[1,5-a]pyridina-1(7,3)-indola-6(3,5)-pyrazola-9(3,1)-naphthalenacyclotridecaphane-12-carboxylic acid (27A, 21.5 mg, 73%, 0.04 mmol) as an off-white solid. MS (ESI) m/z 663.11 [M+H]⁺. ¹H NMR (400 MHz, DMSO-d₆) δ 13.40 (br s, 1H), 9.30 (br s, 1H), 8.90 (br s, 1H), 8.08 (d, J=8.0 Hz, 1H), 7.82 (d, J=8.4 Hz, 1H), 7.71 (d, J=7.6 Hz, 1H), 7.47-7.39 (m, 2H), 7.20 (s, 1H), 7.11 (d, J=8.4 Hz, 1H), 6.28 (s, 1H), 5.17 (s, 1H), 4.19 (t, J=6 Hz, 2H), 3.90-3.88 (m, 1H), 3.77-3.63 (m, 4H), 3.55-3.51 (m, 9H), 3.10-3.08 (s, 2H), 3.07-3.0 (m, 2H), 2.98-2.97 (m, 1H), 2.49-2.33 (m, 2H), 2.32-2.30 (m, 1H), 2.03-2.01 (m, 2H), 1.81-1.78 (m, 2H). LCMS purity: 98.51%; HPLC purity: 98.55%; Chiral HPLC purity: 99.89%.

To a stirred solution of methyl (Z)-1⁶-chloro-1¹,6¹-dimethyl-2⁴,2⁵,2⁶,2⁷-tetrahydro-1¹H,6¹H-10-oxa-4-aza-2(3,2)-pyrazolo[1,5-a]pyridina-1(7,3)-indola-6(3,5)-pyrazola-9(3,1)-naphthalenacyclotridecaphane-12-carboxylate (27-6-peak-2, 28 mg, 0.0414 mmol) in MeOH:THF:H₂O (1:1:1, 3 mL) was added LiOH.H₂O (26 mg, 0.62 mmol), and the mixture was heated at 60° C. for 1 h. The solvent was evaporated, and the aqueous layer was acidified to pH 2 using 2 N aq. HCl. The solid was filtered off, washed with water (5 mL), dried under vacuum to afford (Z)-1⁶-chloro-1¹,6¹-dimethyl-2⁴,2⁵,2⁶,2⁷-tetrahydro-1¹H,6¹H-10-oxa-4-aza-2(3,2)-pyrazolo[1,5-a]pyridina-1(7,3)-indola-6(3,5)-pyrazola-9(3,1)-naphthalenacyclotridecaphane-12-carboxylic acid (27B, 16.5 mg, 61%, 0.025 mmol) as an off-white solid. MS (ESI) m/z 663.15 [M+H]⁺. ¹H NMR (400 MHz, DMSO-d₆) δ 1340 (br s, 1H), 9.30 (br s, 1H), 8.90 (br s, 1H), 8.08 (d, J=8.0 Hz, 1H), 7.82 (d, J=8.4 Hz, 1H), 7.71 (d, J=7.6 Hz, 1H), 7.47-7.39 (m, 2H), 7.19 (s, 1H), 7.11 (d, J=8.4 Hz, 1H), 6.29 (s, 1H), 5.16 (s, 1H), 4.18 (t, J=6 Hz, 2H), 3.91-3.89 (m, 1H), 3.76-3.63 (m, 4H), 3.63-3.51 (m, 9H), 3.33-3.08 (m, 2H), 3.04 (t, 2H), 2.97-2.90 (m, 1H), 2.43-2.33 (m, 2H), 2.32-2.03 (m, 1H), 2.03-2.0 (m, 2H), 1.81-1.78 (m, 2H). LCMS purity: 97.60%; HPLC purity: 97.05%; Chiral HPLC purity: 97.50%.

The absolute stereochemistry of compounds (27A) and (27B) is arbitrarily assigned.

### Example A

### Mcl-1 Homogeneous Time Resolved Fluorescence (HTRF) Assay

Binding to Bcl-2 proteins Mcl-1 was assessed using an HTRF assay. Background: FAM-Bak/Bad binds to surface pocket of the Bcl-2 protein family. This binding can be monitored by HTRF signals between anti-GST-Tb and FAM-peptide using GST-tagged Bcl proteins. Assay conditions: 4 nM Mcl-1, 100 nM FAM-Bak peptide, in 20 mM K Phosphate, pH 7.5, 50 mM NaCl, 1 mM EDTA, 0.005% Triton X-100 and 1% DMSO (final). Assay procedure: Compounds were tested in 10-dose IC₅₀ mode, in singlicate, with 3-fold serial dilution starting at 10 μM or 1 μM. Compound stock solutions were added to protein solution using Acoustic technology. The compounds were then incubated with protein for 10 min at rt. The respective FAM labeled peptide was added and incubated for another 10 min. Anti-GST-Tb was added. After 60 min at rt, the HTRF fluorescence signal ratio was measured. Curve fits were performed in GraphPad Prism 4 with “sigmoidal dose-response (variable slope)”; 4 parameters with Hill Slope. The results are shown in Table 1.

### Example B

### NCI-H929 Cell Proliferation Assay

Cell proliferation was measured using the CellTiter-Glo® Luminescent Cell Viability Assay. The assay involved the addition of a single reagent (CellTiter-Glo® Reagent) directly to cells cultured in serum-supplemented medium. NCI-H929 (ATCC CRL-9068) cells were cultured according to ATCC recommendations and were seeded at 3,000 cells per well.

Each compound evaluated was prepared as a DMSO stock solution (10 mM). Compounds were tested in duplicate on each plate, with a 10-point serial dilution curve (1:3 dilution). Compound treatment (1.0 μL) was added from the compound dilution plate to the cell plate. The highest compound concentration was 10 μM (final), with a 0.1% final DMSO concentration. Plates were then incubated at 37° C., 5% CO₂. After 72 h of compound treatment, cell plates were equilibrated at rt for approximately 30 mins. An equi-volume amount of CellTiter-Glo® Reagent (40 μL) was added to each well. Plates were mixed for 2 mins on an orbital shaker to induce cell lysis and then incubated at rt for 10 mins to stabilize the luminescent signal. Luminescence was recorded using an Envision plate reader according to CellTiter-Glo protocol. IC₅₀ of each compound was calculated using GraphPad Prism by nonlinear regression analysis. IC₅₀ values are provided in Table 1.

TABLE 1

| Examples | Mcl-1 IC₅₀ (nM) | H929 IC₅₀ (nM) |
|---|---|---|
| 1A | A | B |
| 1B | C | C |
| 2A | A | A |
| 2B | B | C |
| 3A | A | A |
| 3B | B | C |
| 4A | A | B |
| 4B | B | C |
| 5A | A | B |
| 5B | C | C |
| 6A | A | A |
| 6B | B | C |
| 7B | A | B |
| 7B | ND | C |
| 8A | ND | C |
| 8B | ND | B |
| 9A | A | B |
| 9B | ND | C |
| 10A | A | A |
| 10B | ND | ND |
| 11A | ND | C |
| 11B | A | A |
| 12A | ND | B |
| 12B | ND | ND |
| 13A | ND | C |
| 13B | ND | C |
| 14A | A | A |
| 14B | ND | C |
| 15A | C | C |
| 15B | ND | C |
| 16A | A | A |
| 16B | ND | C |
| 17A | C | C |
| 17B | ND | B |
| 18A | C | C |
| 18B | ND | B |
| 19A | ND | C |
| 19B | ND | A |
| 20A | B | C |
| 20B | A | A |
| 21A | ND | C |
| 21B | A | A |
| 22A | ND | B |
| 22B | ND | C |
| 23A | ND | B |
| 23B | X | B |
| 24A | ND | B |
| 24B | ND | ND |
| 25A | ND | B |
| 25B | ND | ND |
| 26A | ND | B |
| 26B | ND | C |
| 27A | ND | B |
| 27B | ND | C |
| AMG176 | A | B |
| AZD5991 | A | A |
| S64315 | A | A |

Mcl-1 Binding Assay (IC₅₀): A = a single IC₅₀ ≤10 nM; B = a single IC₅₀ >10 nM and <100 nM; C = a single IC₅₀ ≥100 nM.
For H929 CTG IC₅₀: A = a single IC₅₀ ≤100 nM; B = a single IC₅₀ >100 nM and <1000 nM; C = a single IC₅₀ ≥1000 nM.

Furthermore, although the foregoing has been described in some detail by way of illustrations and examples for purposes of clarity and understanding, it will be understood by those of skill in the art that numerous and various modifications can be made without departing from the spirit of the present disclosure. Therefore, it should be clearly understood that the forms disclosed herein are illustrative only and are not intended to limit the scope of the present disclosure, but rather to also cover all modification and alternatives coming with the true scope and spirit of the invention.

1. A compound of Formula (I), or a pharmaceutically salt thereof, wherein the compound has the structure: 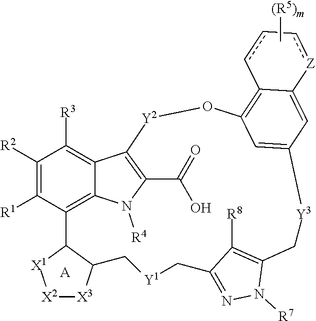
R¹, R², R³ and R⁶ are each independently hydrogen, halogen, an unsubstituted C₁₋₄ alkyl or an unsubstituted C₁₋₄ haloalkyl;
R⁴ and R⁷ are each independently hydrogen, an optionally substituted C₁₋₄ alkyl, an optionally substituted C₃₋₆ monocyclic cycloalkyl or an unsubstituted C₁₋₄ haloalkyl;
X¹, X² and X³ are each independently NR⁸ or CR⁹; and wherein Ring A is an aromatic ring;
R⁸ and R⁹ are each independently absent, hydrogen, halogen, cyano, an optionally substituted C₁₋₄ alkyl, an optionally substituted C₁₋₄ alkoxy, an optionally substituted C₃₋₆ monocyclic cycloalkyl, an optionally substituted C₃₋₆ bicyclic cycloalkyl, a monosubstituted amine, a disubstituted amine or a nitrogen protecting group; or
the substituent attached to X¹ and the substituted attached to X² are taken together to form Ring B fused to Ring A; X³ is NR⁸ or CR⁹, wherein R⁸ and R⁹ are each independently absent, hydrogen, halogen, cyano, an optionally substituted C₁₋₄ alkyl, an optionally substituted C₁₋₄ alkoxy, an optionally substituted C₃₋₆ monocyclic cycloalkyl, an optionally substituted C₃₋₆ bicyclic cycloalkyl, a monosubstituted amine, a disubstituted amine or a nitrogen protecting group; and wherein Ring A and Ring B together form an optionally substituted bicyclic heteroaryl or an optionally substituted bicyclic heterocyclyl; or
the substituent attached to X² and the substituted attached to X³ are taken together to form Ring C fused to Ring A; X¹ is NR⁸ or CR⁹, wherein R⁸ and R⁹ are each independently absent, hydrogen, halogen, cyano, an optionally substituted C₁₋₄ alkyl, an optionally substituted C₁₋₄ alkoxy, an optionally substituted C₃₋₆ monocyclic cycloalkyl, an optionally substituted C₃₋₆ bicyclic cycloalkyl, a monosubstituted amine, a disubstituted amine or a nitrogen protecting group; and wherein Ring A and Ring C together form an optionally substituted bicyclic heteroaryl or an optionally substituted bicyclic heterocyclyl;
Y¹ is O, S, SO, SO₂, CH₂, CF₂ or NR^{10A};
Y² is an optionally substituted C₁₋₄ alkylene, and when Y² is substituted, each substituent is independently halogen or an unsubstituted C₁₋₄ alkyl;
Y³ is O, S, SO, SO₂, CH₂, CF₂ or NR^{10B};
R^{10A} and R^{10B} are independently hydrogen or an optionally substituted C₁₋₄ alkyl;
Z is CH₂, CH or NH, wherein when Z is CH₂, then each [symbol] is a single bond; wherein when Z is CH, then each [symbol] is a double bond; and wherein when Z is NH, then each [symbol] is a single bond;
m is 0, 1 or 2; and
each R⁵ is independently halogen or an optionally substituted C₁₋₄ alkyl; and
provided that
when Y¹, Y² and Y³ are:
(1) Y¹ and Y³ are each S and Y² is —(CH₂)₃—;
(2) Y¹ is S, Y² is —(CH₂)₃— and Y³ is —(CH₂)—;
(3) Y¹ is NH, NCH₃ or NCH₂CH₃, Y² is —(CH₂)₃— and Y³ is S; or
(4) Y¹ is NH, NCH₃ or NCH₂CH₃, Y² is —(CH₂)₃— and Y³ is —(CH₂)—;
R¹ is chloro;
R², R³ and R⁶ are each hydrogen;
R⁴ and R⁷ are each methyl;
Z is CH and each [symbol] is a double bond; and m is 0;
then X¹, X² and X³ are not the following: X¹ is CR⁸, wherein R⁸ is methyl, X² is N and X³ is N(CH₃); and
provided that the compound is not 17-chloro-5,13,14,22-tetramethyl-28-oxa-2,9-dithia-5,6,12,13,22-pentaazaheptocyclo[27.7.1.^{14,7}.0^{11,15}.0^{16,21}.0^{20,24}.0^{30,35}]octatriaconta-1(37),4(38),6,11,14,16,18,20,23,29,31,33,35-tridecaene-23-carboxylic acid, or a pharmaceutically acceptable salt thereof.

2. (canceled)

3. The compound of claim 1, wherein R¹ is halogen; R² is hydrogen; R³ is hydrogen; and R⁴ is an unsubstituted C₁₋₄ alkyl or an unsubstituted C₃₋₆ monocyclic cycloalkyl.

4.-32. (canceled)

33. The compound of claim 1, wherein R⁶ is hydrogen; and R⁷ is an unsubstituted C₁₋₄ alkyl.

34.-43. (canceled)

44. The compound of claim 1, wherein X¹, X² and X³ are each independently NR⁸ or CR⁹; Ring A is a monocyclic aromatic ring; and R⁸ and R⁹ are each independently absent, hydrogen, halogen, cyano, an optionally substituted C₁₋₄ alkyl, an optionally substituted C₁₋₄ alkoxy, an optionally substituted C₃₋₆ monocyclic cycloalkyl, an optionally substituted C₃₋₆ bicyclic cycloalkyl, a monosubstituted amine, a disubstituted amine or a nitrogen protecting group.

45.-48. (canceled)

49. The compound of claim 1, wherein Ring A is

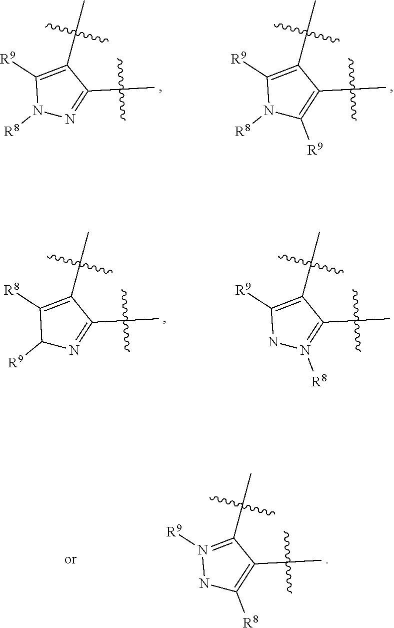

50. (canceled)

51. The compound of claim 1, wherein X¹ and X² are each independently NR⁸ or CR⁹; the substituent attached to X¹ and the substituted attached to X² are taken together to form Ring B fused to Ring A; X³ is NR⁸ or CR⁹; Ring A and Ring B form an optionally substituted heteroaryl or an optionally substituted heterocyclyl; and R⁸ and R⁹ are each independently absent, hydrogen, halogen, cyano, an optionally substituted C₁₋₄ alkyl, an optionally substituted C₁₋₄ alkoxy, an optionally substituted C₃₋₆ monocyclic cycloalkyl, an optionally substituted C₃₋₆ bicyclic cycloalkyl, a monosubstituted amine, a disubstituted amine or a nitrogen protecting group; or wherein X² and X³ are each independently NR⁸ or CR⁹; the substituent attached to X² and the substituted attached to X³ are taken together to form Ring C fused to Ring A: X¹ is NR⁸ or CR⁹; Ring A and Ring C form an optionally substituted heteroaryl or an optionally substituted heterocyclyl; and R⁸ and R⁹ are each independently absent, hydrogen, halogen, cyano, an optionally substituted C₁₋₄ alkyl, an optionally substituted C₁₋₄ alkoxy, an optionally substituted C₃₋₆ monocyclic cycloalkyl, an optionally substituted C₃₋₆ bicyclic cycloalkyl, a monosubstituted amine, a disubstituted amine or a nitrogen protecting group.

52.-57. (canceled)

58. The compound of claim 51, wherein Ring A fused to Ring B are 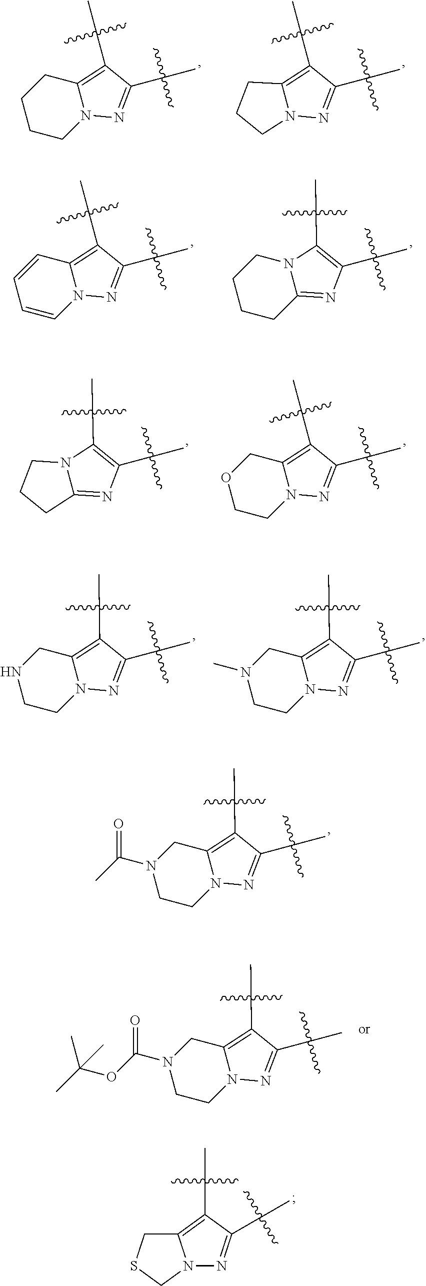
and wherein Ring A fused to Ring C

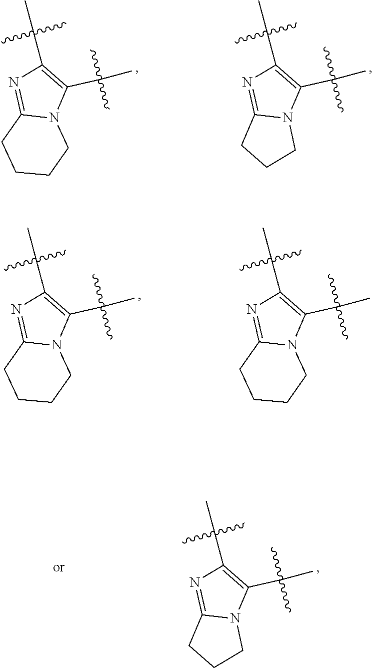 [SMILES: *c1nc2n(c1*)CCCC2 ; *c1nc2n(c1*)CCC2 ; *c1nc2n(c1*)CCCC2 ; *c1nc2n(c1*)CCCC2 ; *c1nc2n(c1*)CCC2]

59.-66. (canceled)

67. The compound of claim 1, wherein Z is CH₂; and each [symbol] are a single bond.

68. The compound of claim 1, wherein Z is CH; and each [symbol] are a double bond.

69. The compound of claim 1, wherein Z is NH; and each [symbol] are a single bond.

70.-79. (canceled)

80. The compound of claim 1, wherein Y¹ is S.

81.-83. (canceled)

84. The compound of claim 1, wherein Y¹ is O, CF₂, or NR^{10A}, wherein R^{10A} is hydrogen or an optionally substituted C₁₋₄ alkyl.

85. (canceled)

86. (canceled)

87. The compound of claim 1, wherein Y² is an unsubstituted C₁₋₄ alkylene.

88.-90. (canceled)

91. The compound of claim 1, wherein Y³ is S.

92.-98. (canceled)

99. The compound of claim 1, wherein the compound is selected from the group consisting of: 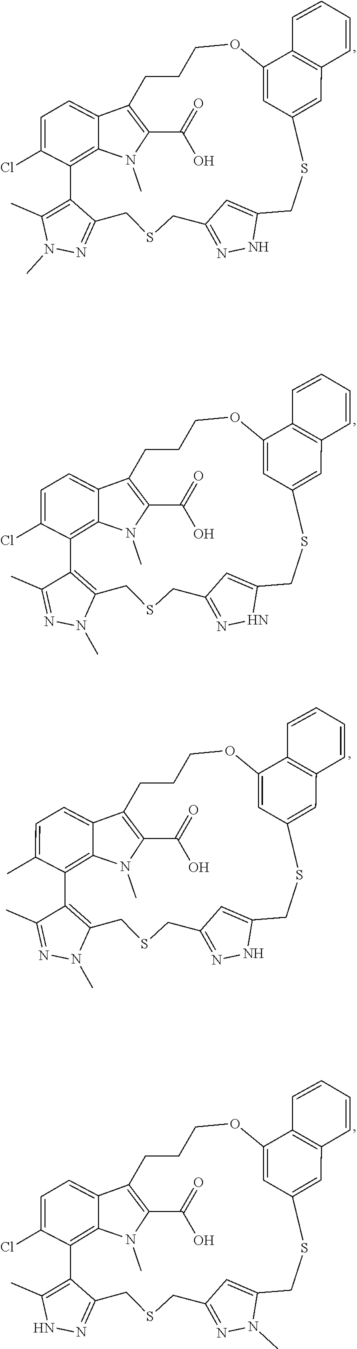 [SMILES: Cc1c2c(nn1C)CSCc1cc([nH]n1)CSc1cc(c3ccccc3c1)OCCCc1c(C(=O)O)n(C)c3c-2c(Cl)ccc13 ; Cc1nn(C)c2c1-c1c(Cl)ccc3c(c(C(=O)O)n(C)c13)CCCOc1cc(cc3ccccc13)SCc1cc(n[nH]1)CSC2 ; Cc1ccc2c3c(C(=O)O)n(C)c2c1-c1c(C)nn(C)c1CSCc1cc([nH]n1)CSc1cc(c2ccccc2c1)OCCC3 ; Cc1[nH]nc2c1-c1c(Cl)ccc3c(c(C(=O)O)n(C)c13)CCCOc1cc(cc3ccccc13)SCc1cc(nn1C)CSC2] 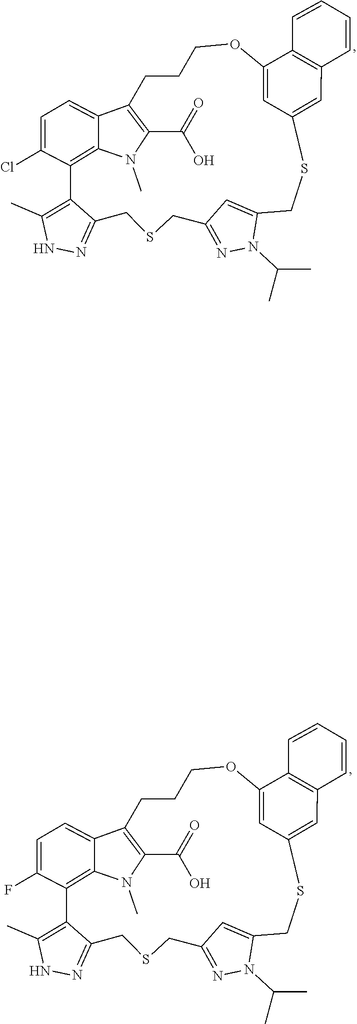 [SMILES: Cc1[nH]nc2c1-c1c(Cl)ccc3c(c(C(=O)O)n(C)c13)CCCOc1cc(cc3ccccc13)SCc1cc(nn1C(C)C)CSC2 ; Cc1[nH]nc2c1-c1c(F)ccc3c(c(C(=O)O)n(C)c13)CCCOc1cc(cc3ccccc13)SCc1cc(nn1C(C)C)CSC2] 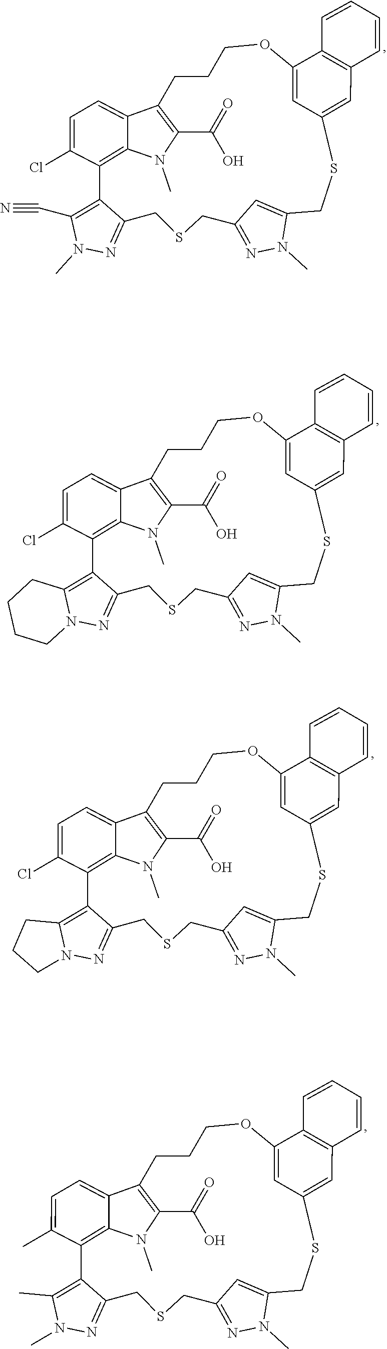 [SMILES: Cn1nc2cc1CSc1cc(c3ccccc3c1)OCCCc1c(C(=O)O)n(C)c3c(c(Cl)ccc13)-c1c(nn(C)c1C#N)CSC2 ; Cn1nc2cc1CSc1cc(c3ccccc3c1)OCCCc1c(C(=O)O)n(C)c3c(c(Cl)ccc13)-c1c(nn3c1CCCC3)CSC2 ; Cn1nc2cc1CSc1cc(c3ccccc3c1)OCCCc1c(C(=O)O)n(C)c3c(c(Cl)ccc13)-c1c(nn3c1CCC3)CSC2 ; Cc1ccc2c3c(C(=O)O)n(C)c2c1-c1c(nn(C)c1C)CSCc1cc(n(C)n1)CSc1cc(c2ccccc2c1)OCCC3] 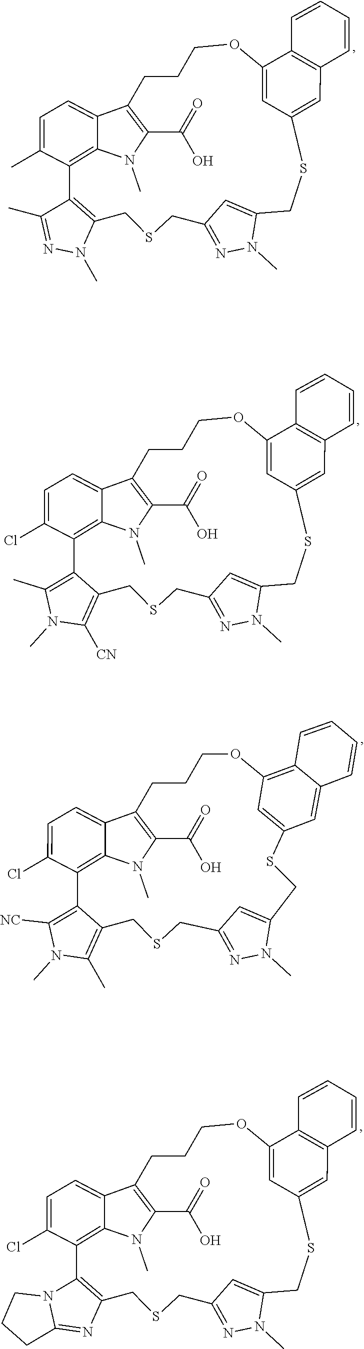 [SMILES: Cc1ccc2c3c(C(=O)O)n(C)c2c1-c1c(C)nn(C)c1CSCc1cc(n(C)n1)CSc1cc(c2ccccc2c1)OCCC3 ; Cc1c2c(c(C#N)n1C)CSCc1cc(n(C)n1)CSc1cc(c3ccccc3c1)OCCCc1c(C(=O)O)n(C)c3c-2c(Cl)ccc13 ; Cc1c2c(c(C#N)n1C)-c1c(Cl)ccc3c(c(C(=O)O)n(C)c13)CCCOc1cc(cc3ccccc13)SCc1cc(nn1C)CSC2 ; Cn1nc2cc1CSc1cc(c3ccccc3c1)OCCCc1c(C(=O)O)n(C)c3c(c(Cl)ccc13)-c1c(nc3n1CCC3)CSC2] 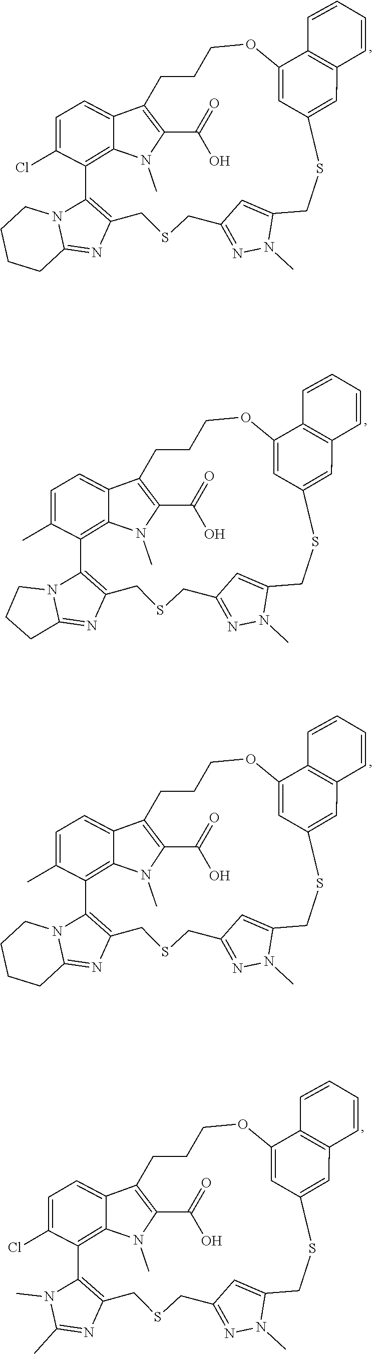 [SMILES: Cn1nc2cc1CSc1cc(c3ccccc3c1)OCCCc1c(C(=O)O)n(C)c3c(c(Cl)ccc13)-c1c(nc3n1CCCC3)CSC2 ; Cc1ccc2c3c(C(=O)O)n(C)c2c1-c1c(nc2n1CCC2)CSCc1cc(n(C)n1)CSc1cc(c2ccccc2c1)OCCC3 ; Cc1ccc2c3c(C(=O)O)n(C)c2c1-c1c(nc2n1CCCC2)CSCc1cc(n(C)n1)CSc1cc(c2ccccc2c1)OCCC3 ; Cc1nc2c(n1C)-c1c(Cl)ccc3c(c(C(=O)O)n(C)c13)CCCOc1cc(cc3ccccc13)SCc1cc(nn1C)CSC2] 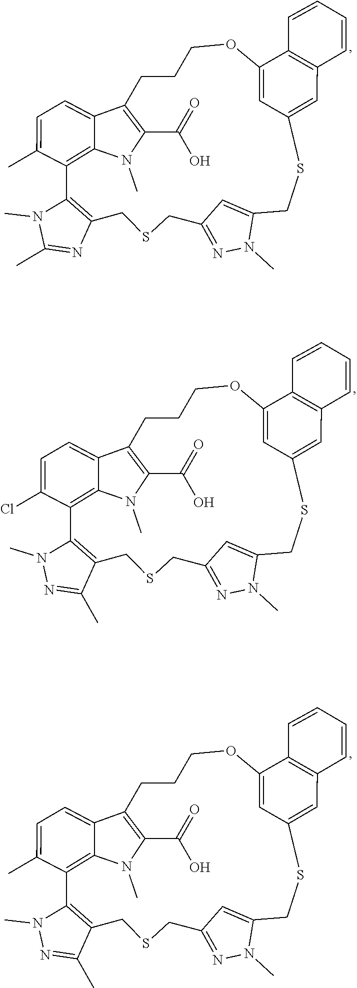 [SMILES: Cc1ccc2c3c(C(=O)O)n(C)c2c1-c1c(nc(C)n1C)CSCc1cc(n(C)n1)CSc1cc(c2ccccc2c1)OCCC3 ; Cc1nn(C)c2c1CSCc1cc(n(C)n1)CSc1cc(c3ccccc3c1)OCCCc1c(C(=O)O)n(C)c3c-2c(Cl)ccc13 ; Cc1ccc2c3c(C(=O)O)n(C)c2c1-c1c(c(C)nn1C)CSCc1cc(n(C)n1)CSc1cc(c2ccccc2c1)OCCC3] 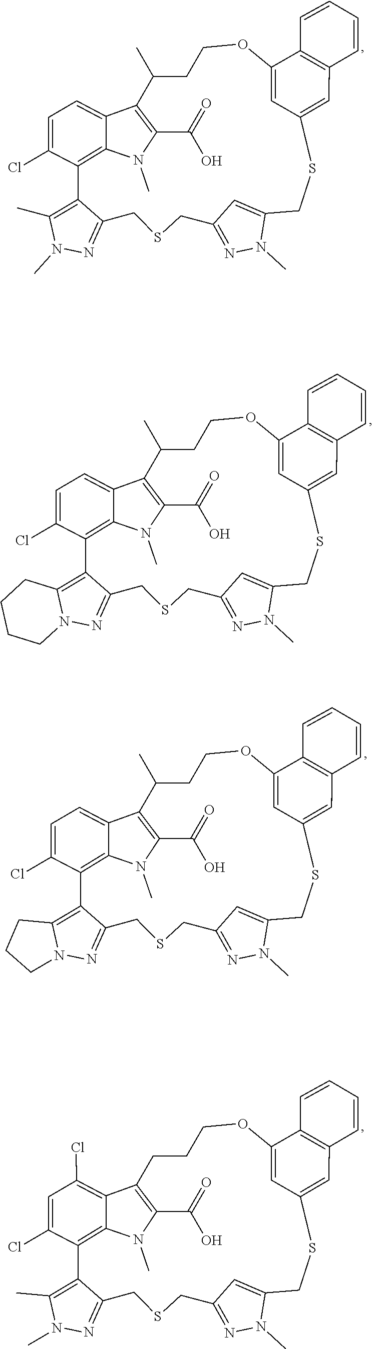 [SMILES: Cc1c2c(nn1C)CSCc1cc(n(C)n1)CSc1cc(c3ccccc3c1)OCCC(C)c1c(C(=O)O)n(C)c3c-2c(Cl)ccc13 ; CC1CCOc2cc(cc3ccccc23)SCc2cc(nn2C)CSCc2nn3c(c2-c2c(Cl)ccc4c1c(C(=O)O)n(C)c24)CCCC3 ; CC1CCOc2cc(cc3ccccc23)SCc2cc(nn2C)CSCc2nn3c(c2-c2c(Cl)ccc4c1c(C(=O)O)n(C)c24)CCC3 ; Cc1c2c(nn1C)CSCc1cc(n(C)n1)CSc1cc(c3ccccc3c1)OCCCc1c(C(=O)O)n(C)c3c-2c(Cl)cc(Cl)c13] 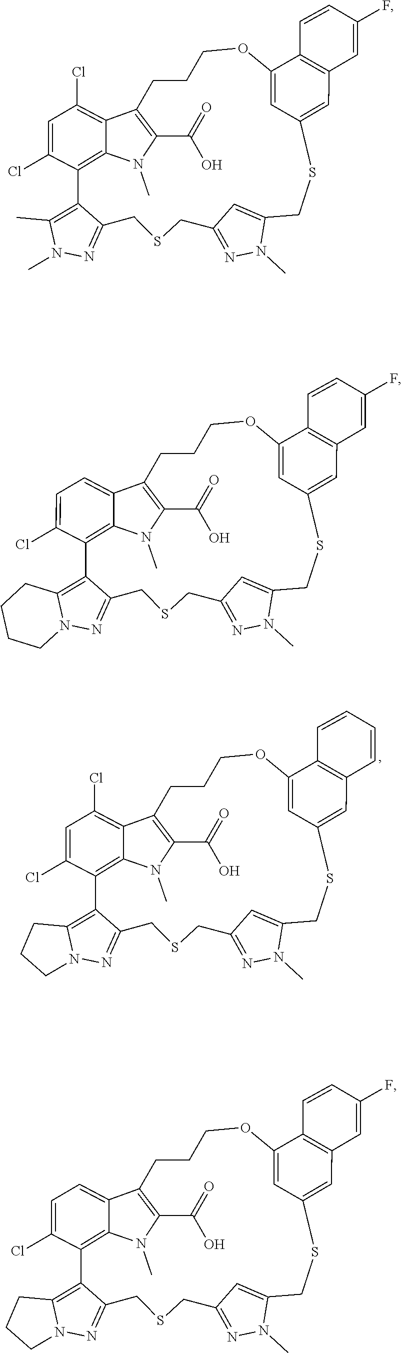 [SMILES: Cc1c2c(nn1C)CSCc1cc(n(C)n1)CSc1cc(c3ccc(F)cc3c1)OCCCc1c(C(=O)O)n(C)c3c-2c(Cl)cc(Cl)c13 ; Cn1nc2cc1CSc1cc(c3ccc(F)cc3c1)OCCCc1c(C(=O)O)n(C)c3c(c(Cl)ccc13)-c1c(nn3c1CCCC3)CSC2 ; Cn1nc2cc1CSc1cc(c3ccccc3c1)OCCCc1c(C(=O)O)n(C)c3c(c(Cl)cc(Cl)c13)-c1c(nn3c1CCC3)CSC2 ; Cn1nc2cc1CSc1cc(c3ccc(F)cc3c1)OCCCc1c(C(=O)O)n(C)c3c(c(Cl)ccc13)-c1c(nn3c1CCC3)CSC2] 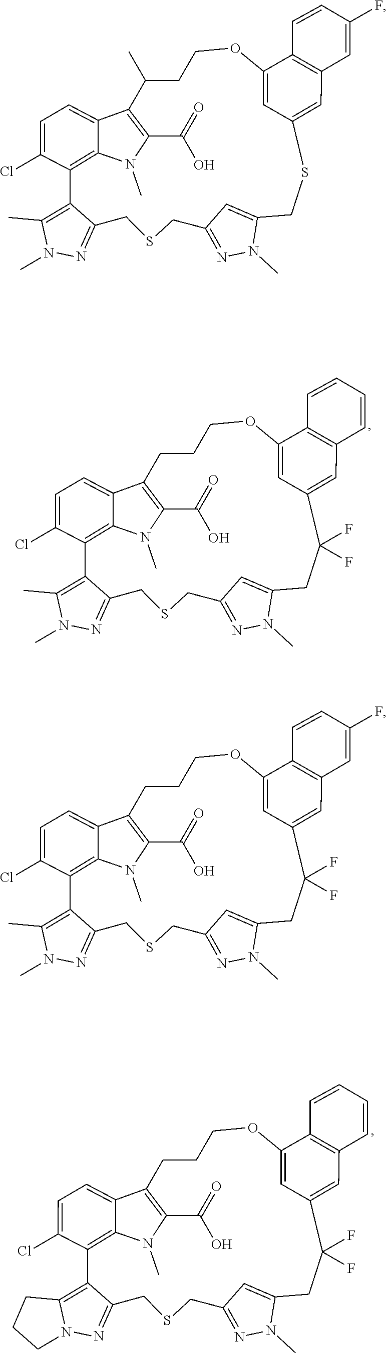 [SMILES: Cc1c2c(nn1C)CSCc1cc(n(C)n1)CSc1cc(c3ccc(F)cc3c1)OCCC(C)c1c(C(=O)O)n(C)c3c-2c(Cl)ccc13 ; Cc1c2c(nn1C)CSCc1cc(n(C)n1)CC(F)(F)c1cc(c3ccccc3c1)OCCCc1c(C(=O)O)n(C)c3c-2c(Cl)ccc13 ; Cc1c2c(nn1C)CSCc1cc(n(C)n1)CC(F)(F)c1cc(c3ccc(F)cc3c1)OCCCc1c(C(=O)O)n(C)c3c-2c(Cl)ccc13 ; Cn1nc2cc1CC(F)(F)c1cc(c3ccccc3c1)OCCCc1c(C(=O)O)n(C)c3c(c(Cl)ccc13)-c1c(nn3c1CCC3)CSC2] 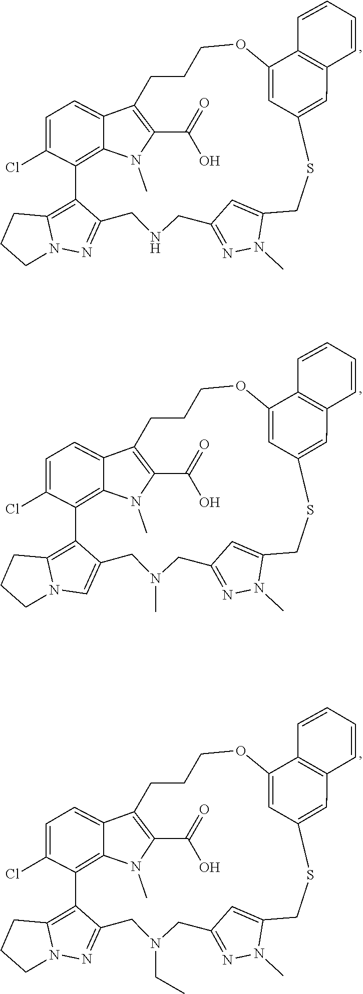 [SMILES: Cn1nc2cc1CSc1cc(c3ccccc3c1)OCCCc1c(C(=O)O)n(C)c3c(c(Cl)ccc13)-c1c(nn3c1CCC3)CNC2 ; CN1Cc2cc(n(C)n2)CSc2cc(c3ccccc3c2)OCCCc2c(C(=O)O)n(C)c3c(c(Cl)ccc23)-c2c(cn3c2CCC3)C1 ; CCN1Cc2cc(n(C)n2)CSc2cc(c3ccccc3c2)OCCCc2c(C(=O)O)n(C)c3c(c(Cl)ccc23)-c2c(nn3c2CCC3)C1] 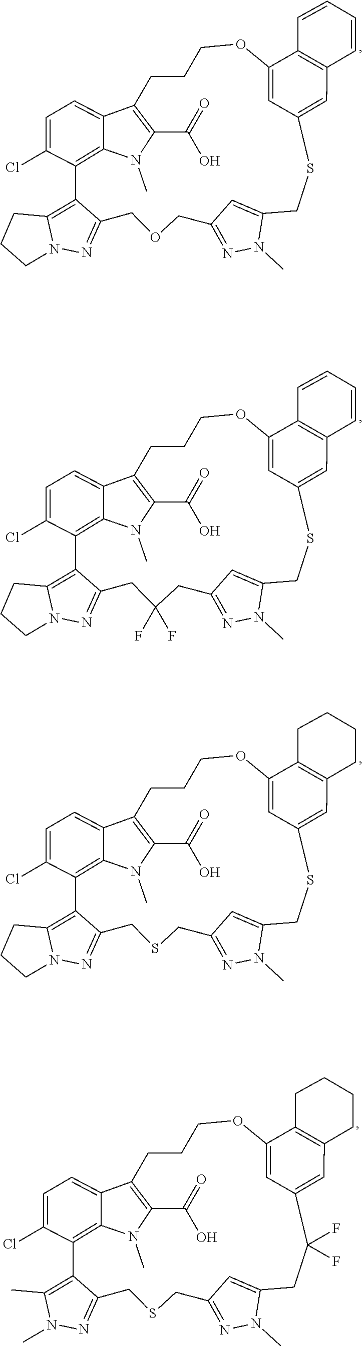 [SMILES: Cn1nc2cc1CSc1cc(c3ccccc3c1)OCCCc1c(C(=O)O)n(C)c3c(c(Cl)ccc13)-c1c(nn3c1CCC3)COC2 ; Cn1nc2cc1CSc1cc(c3ccccc3c1)OCCCc1c(C(=O)O)n(C)c3c(c(Cl)ccc13)-c1c(nn3c1CCC3)CC(F)(F)C2 ; Cn1nc2cc1CSc1cc3c(c(c1)OCCCc1c(C(=O)O)n(C)c4c(c(Cl)ccc14)-c1c(nn4c1CCC4)CSC2)CCCC3 ; Cc1c2c(nn1C)CSCc1cc(n(C)n1)CC(F)(F)c1cc3c(c(c1)OCCCc1c(C(=O)O)n(C)c4c-2c(Cl)ccc14)CCCC3] 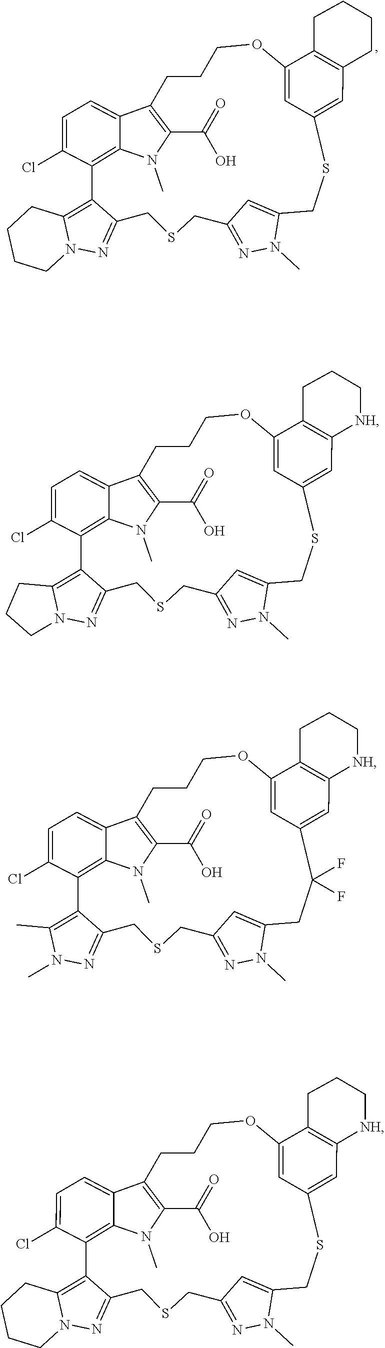 [SMILES: Cn1nc2cc1CSc1cc3c(c(c1)OCCCc1c(C(=O)O)n(C)c4c(c(Cl)ccc14)-c1c(nn4c1CCCC4)CSC2)CCCC3 ; Cn1nc2cc1CSc1cc3c(c(c1)OCCCc1c(C(=O)O)n(C)c4c(c(Cl)ccc14)-c1c(nn4c1CCC4)CSC2)CCCN3 ; Cc1c2c(nn1C)CSCc1cc(n(C)n1)CC(F)(F)c1cc3c(c(c1)OCCCc1c(C(=O)O)n(C)c4c-2c(Cl)ccc14)CCCN3 ; Cn1nc2cc1CSc1cc3c(c(c1)OCCCc1c(C(=O)O)n(C)c4c(c(Cl)ccc14)-c1c(nn4c1CCCC4)CSC2)CCCN3] 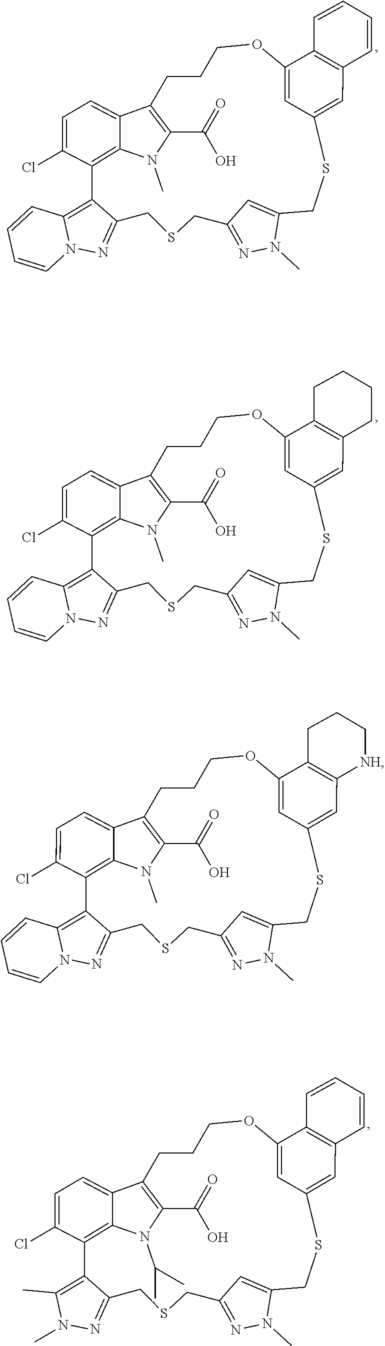 [SMILES: Cn1nc2cc1CSc1cc(c3ccccc3c1)OCCCc1c(C(=O)O)n(C)c3c(c(Cl)ccc13)-c1c(nn3ccccc13)CSC2 ; Cn1nc2cc1CSc1cc3c(c(c1)OCCCc1c(C(=O)O)n(C)c4c(c(Cl)ccc14)-c1c(nn4ccccc14)CSC2)CCCC3 ; Cn1nc2cc1CSc1cc3c(c(c1)OCCCc1c(C(=O)O)n(C)c4c(c(Cl)ccc14)-c1c(nn4ccccc14)CSC2)CCCN3 ; Cc1c2c(nn1C)CSCc1cc(n(C)n1)CSc1cc(c3ccccc3c1)OCCCc1c(C(=O)O)n(C(C)C)c3c-2c(Cl)ccc13] 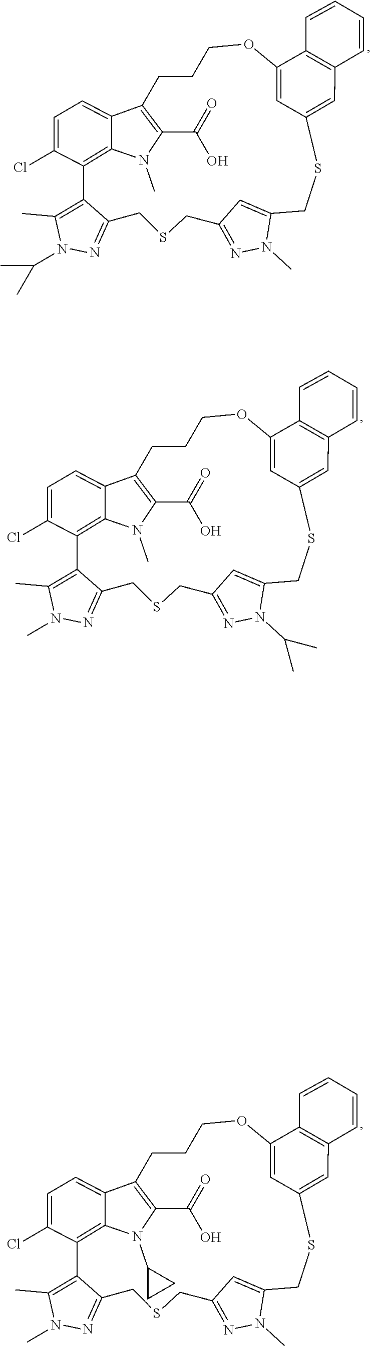 [SMILES: Cc1c2c(nn1C(C)C)CSCc1cc(n(C)n1)CSc1cc(c3ccccc3c1)OCCCc1c(C(=O)O)n(C)c3c-2c(Cl)ccc13 ; Cc1c2c(nn1C)CSCc1cc(n(C(C)C)n1)CSc1cc(c3ccccc3c1)OCCCc1c(C(=O)O)n(C)c3c-2c(Cl)ccc13 ; Cc1c2c(nn1C)CSCc1cc(n(C)n1)CSc1cc(c3ccccc3c1)OCCCc1c(C(=O)O)n(C3CC3)c3c-2c(Cl)ccc13] 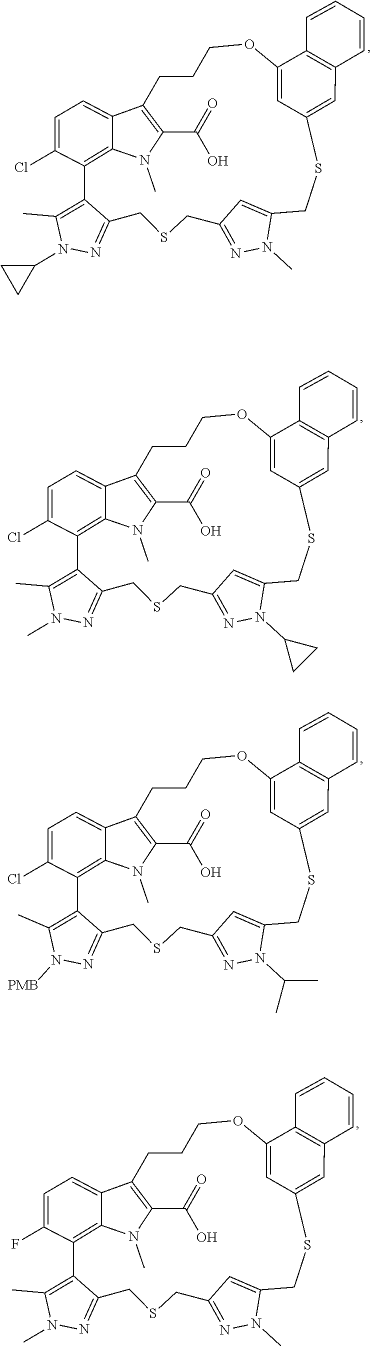 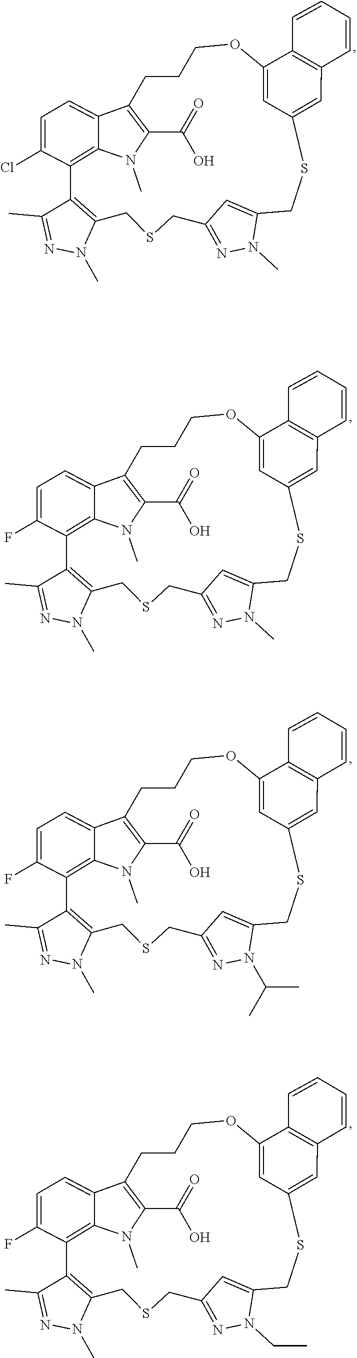 [SMILES: Cc1nn(C)c2c1-c1c(Cl)ccc3c(c(C(=O)O)n(C)c13)CCCOc1cc(cc3ccccc13)SCc1cc(nn1C)CSC2 ; Cc1nn(C)c2c1-c1c(F)ccc3c(c(C(=O)O)n(C)c13)CCCOc1cc(cc3ccccc13)SCc1cc(nn1C)CSC2 ; Cc1nn(C)c2c1-c1c(F)ccc3c(c(C(=O)O)n(C)c13)CCCOc1cc(cc3ccccc13)SCc1cc(nn1C(C)C)CSC2 ; CCn1nc2cc1CSc1cc(c3ccccc3c1)OCCCc1c(C(=O)O)n(C)c3c(c(F)ccc13)-c1c(C)nn(C)c1CSC2] 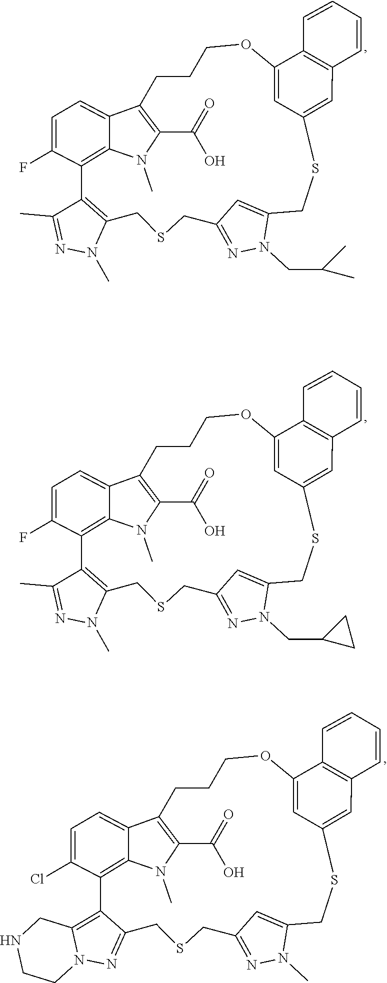 [SMILES: Cc1nn(C)c2c1-c1c(F)ccc3c(c(C(=O)O)n(C)c13)CCCOc1cc(cc3ccccc13)SCc1cc(nn1CC(C)C)CSC2 ; Cc1nn(C)c2c1-c1c(F)ccc3c(c(C(=O)O)n(C)c13)CCCOc1cc(cc3ccccc13)SCc1cc(nn1CC1CC1)CSC2 ; Cn1nc2cc1CSc1cc(c3ccccc3c1)OCCCc1c(C(=O)O)n(C)c3c(c(Cl)ccc13)-c1c(nn3c1CNCC3)CSC2] 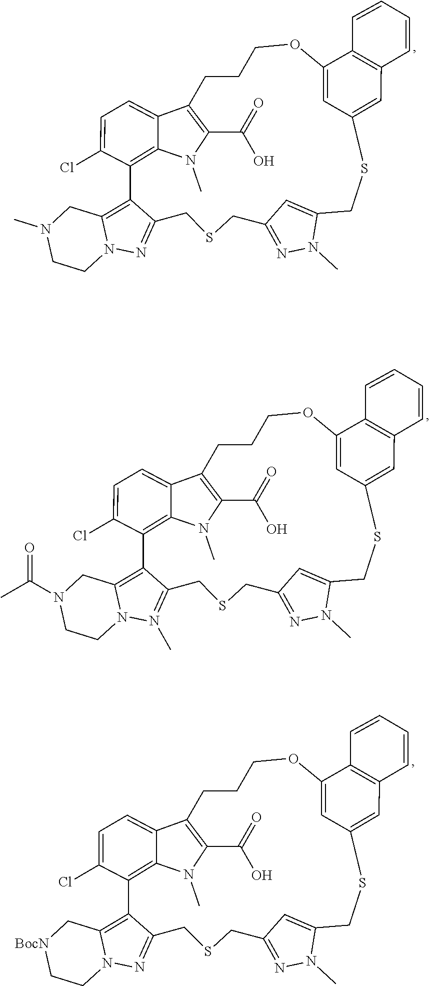 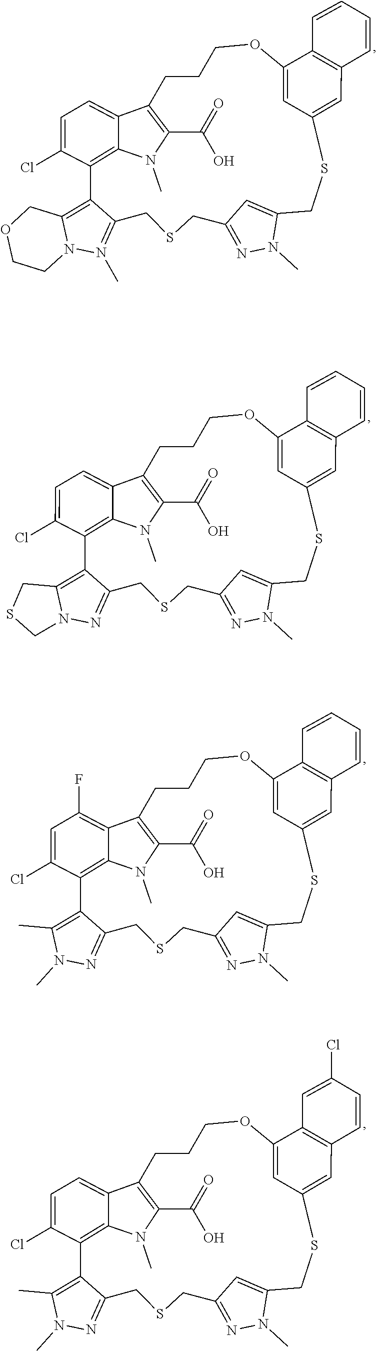 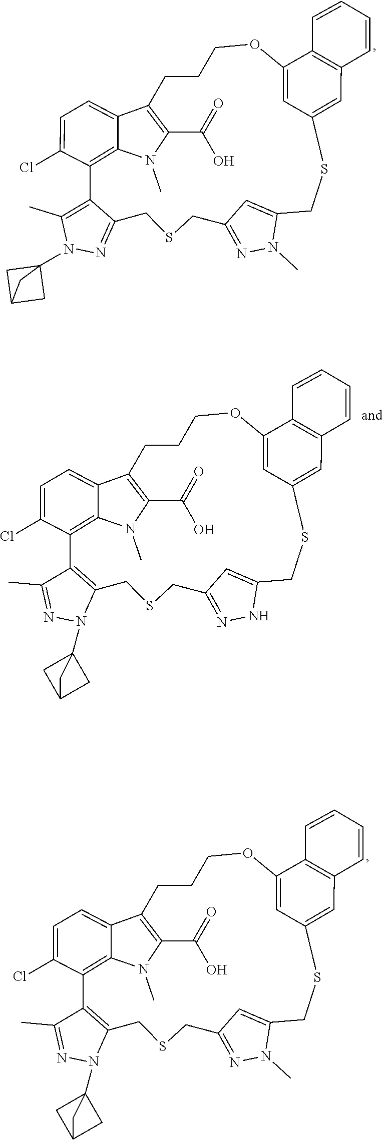 [SMILES: Cc1c2c(nn1C13CC(C1)C3)CSCc1cc(n(C)n1)CSc1cc(c3ccccc3c1)OCCCc1c(C(=O)O)n(C)c3c-2c(Cl)ccc13 ; Cc1nn(C23CC(C2)C3)c2c1-c1c(Cl)ccc3c(c(C(=O)O)n(C)c13)CCCOc1cc(cc3ccccc13)SCc1cc(n[nH]1)CSC2 ; Cc1nn(C23CC(C2)C3)c2c1-c1c(Cl)ccc3c(c(C(=O)O)n(C)c13)CCCOc1cc(cc3ccccc13)SCc1cc(nn1C)CSC2]
or a pharmaceutically acceptable salt of any of the foregoing.

100. The compound of claim 99, wherein the compound is selected from the group consisting of: 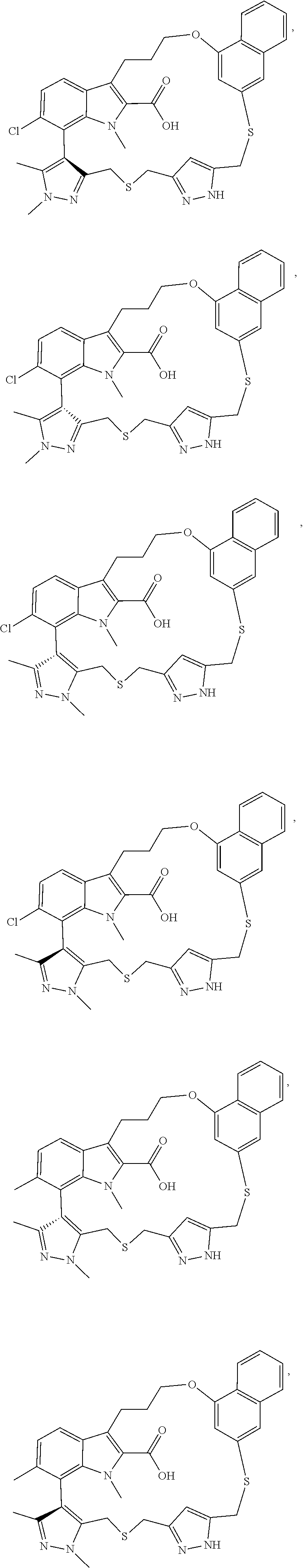 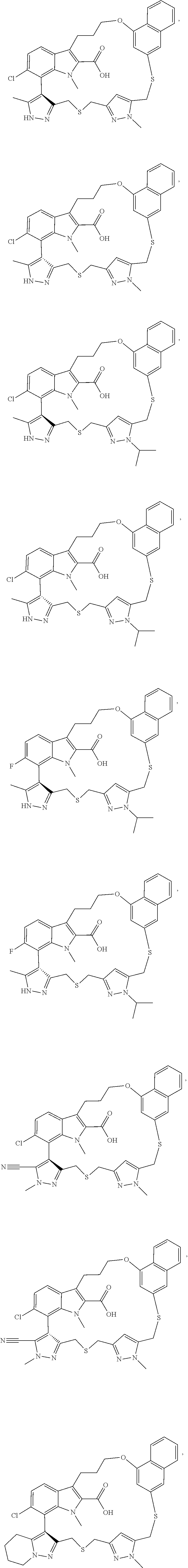 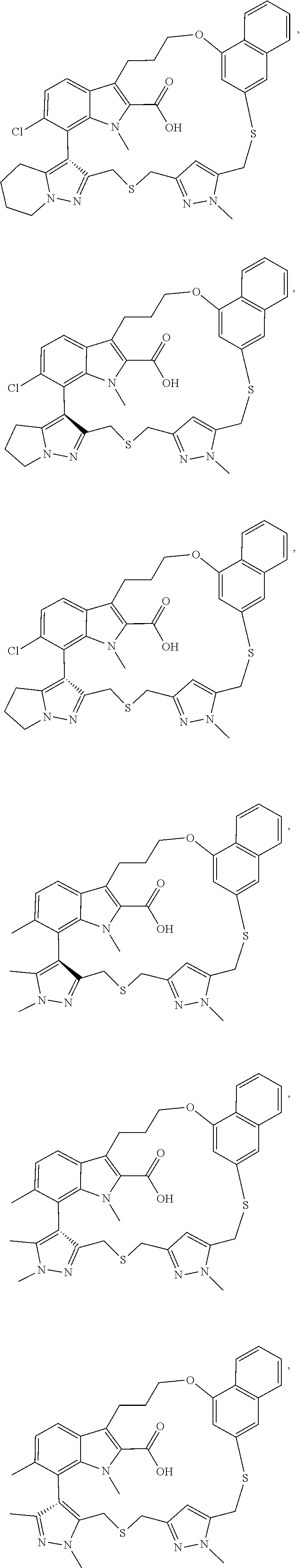 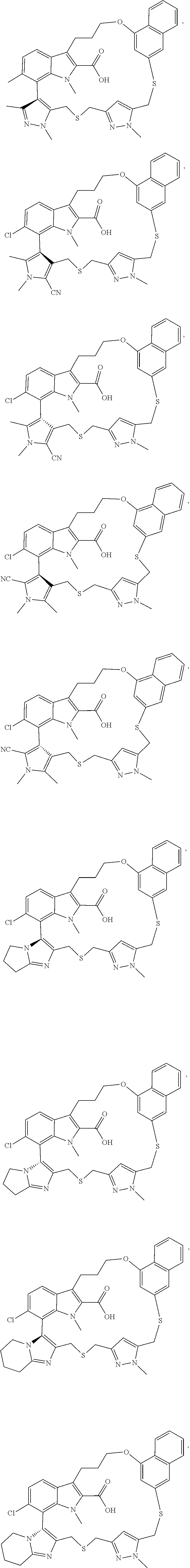 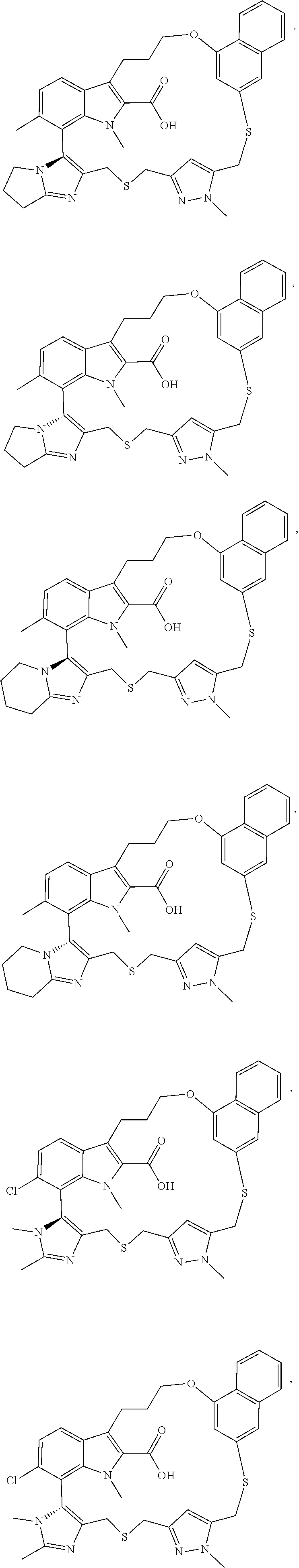 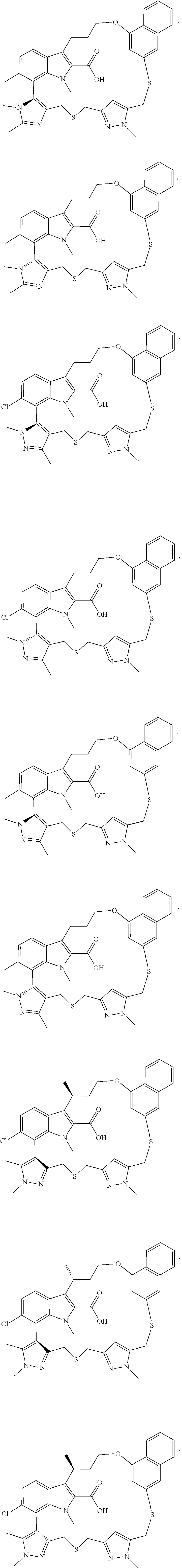 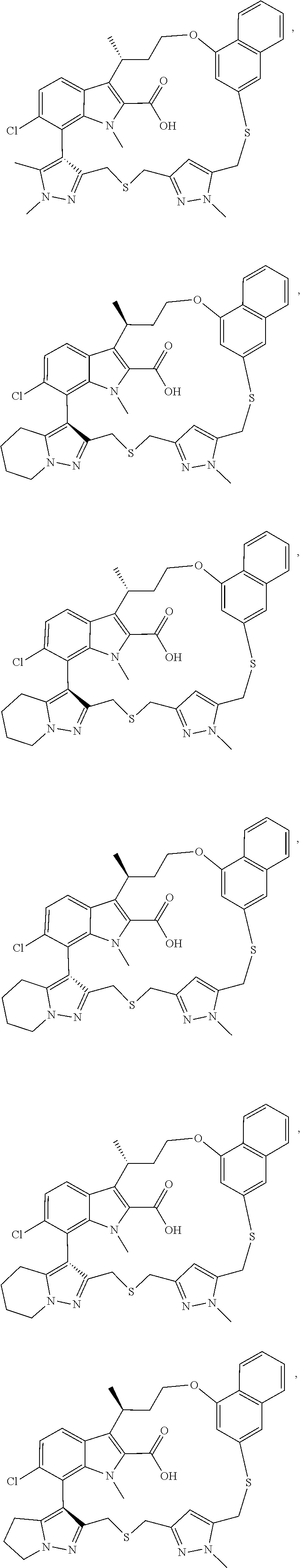 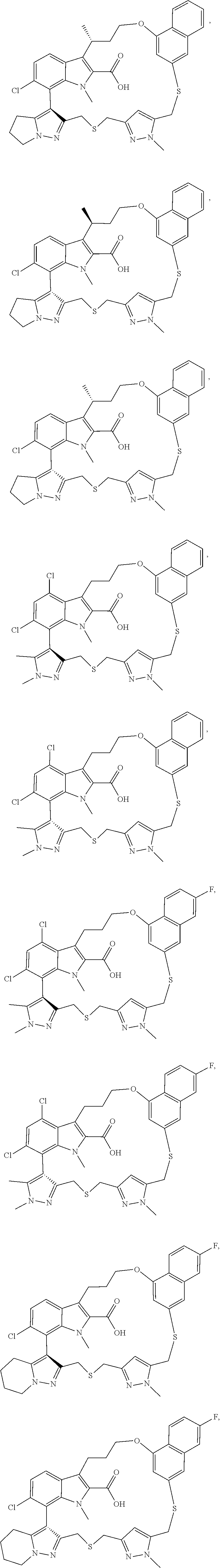 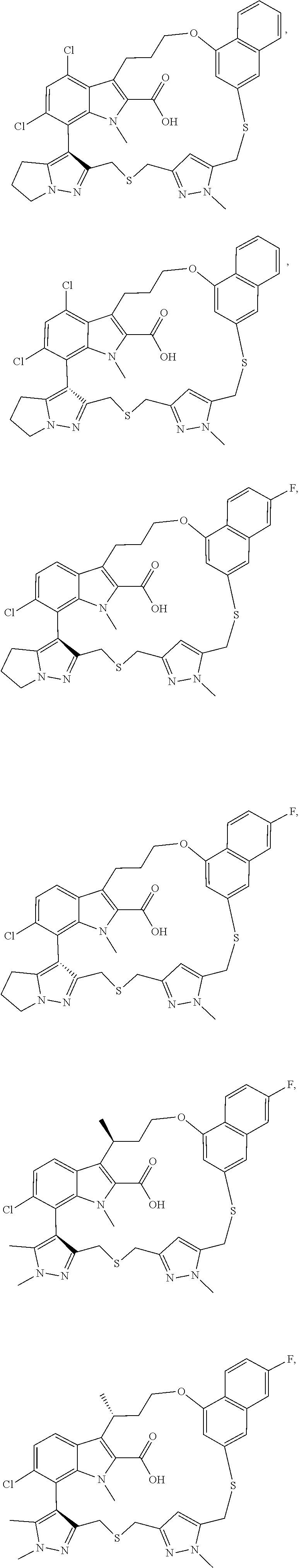 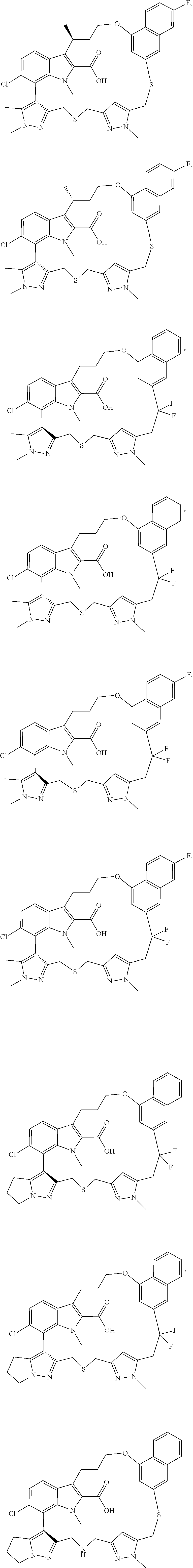 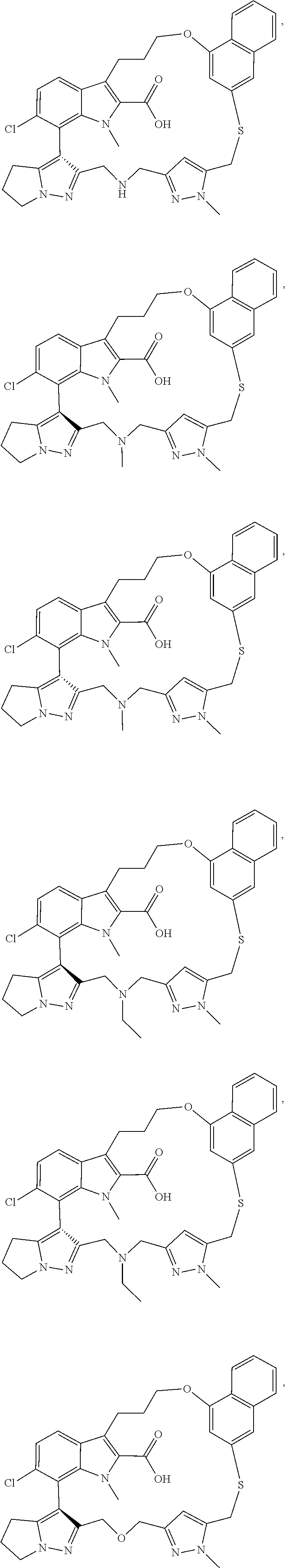 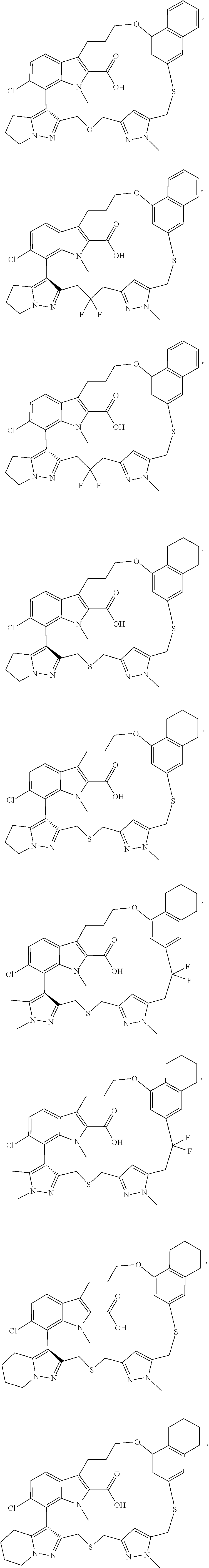 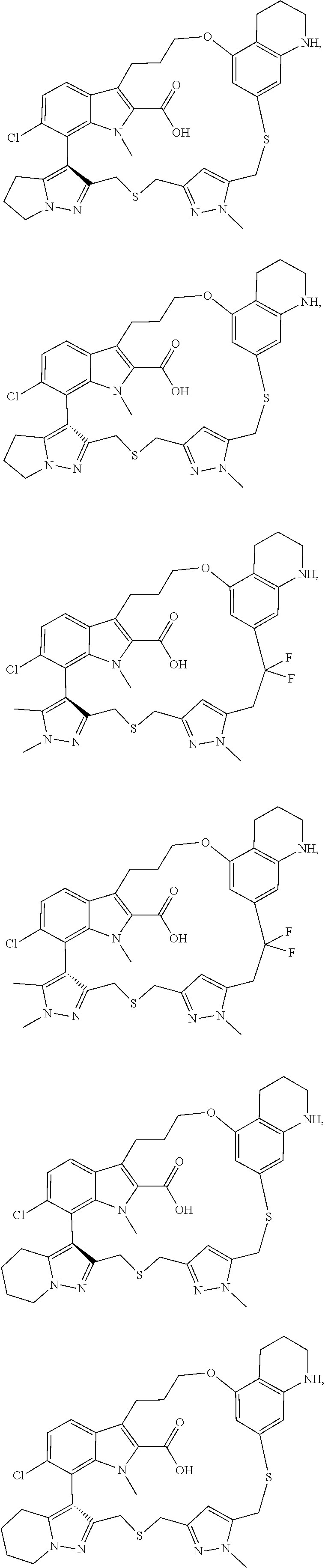 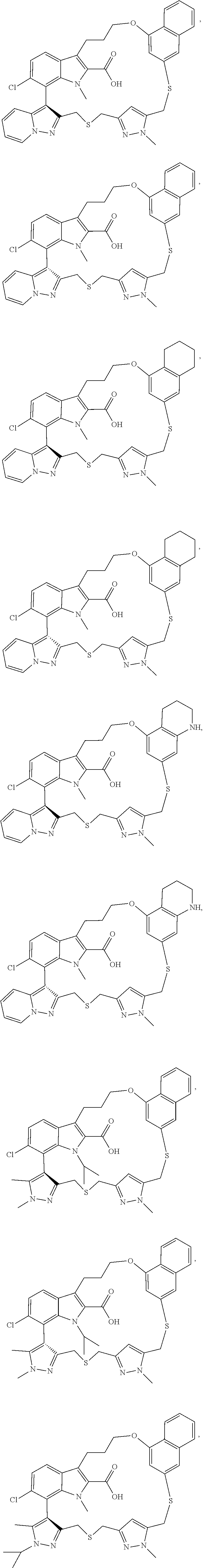 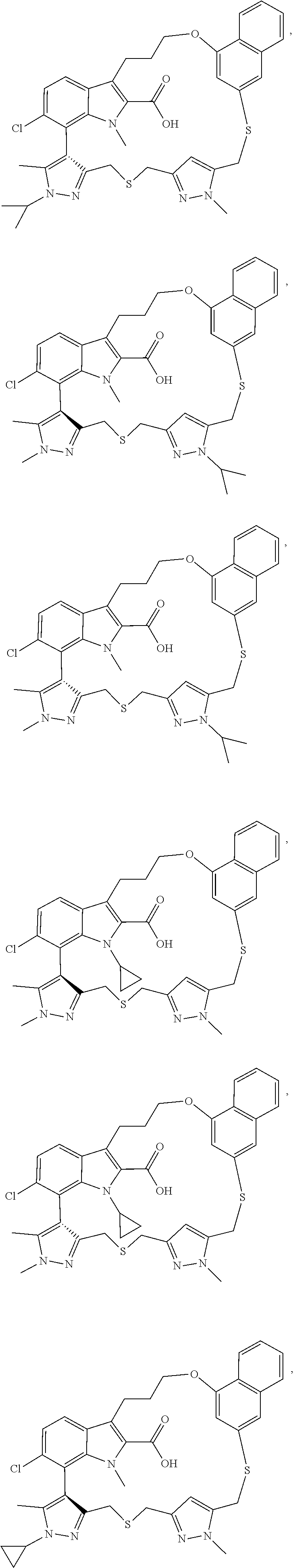 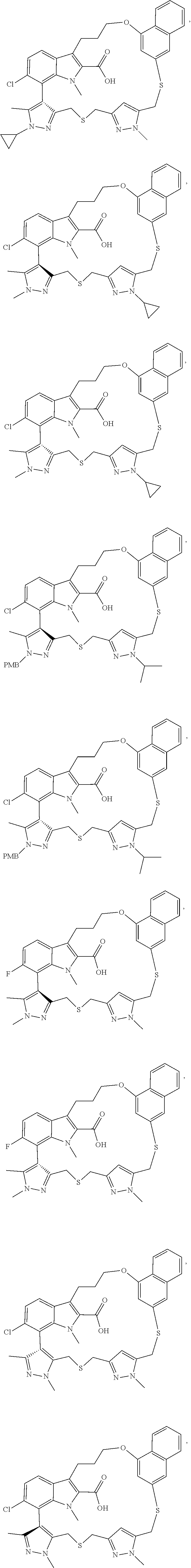 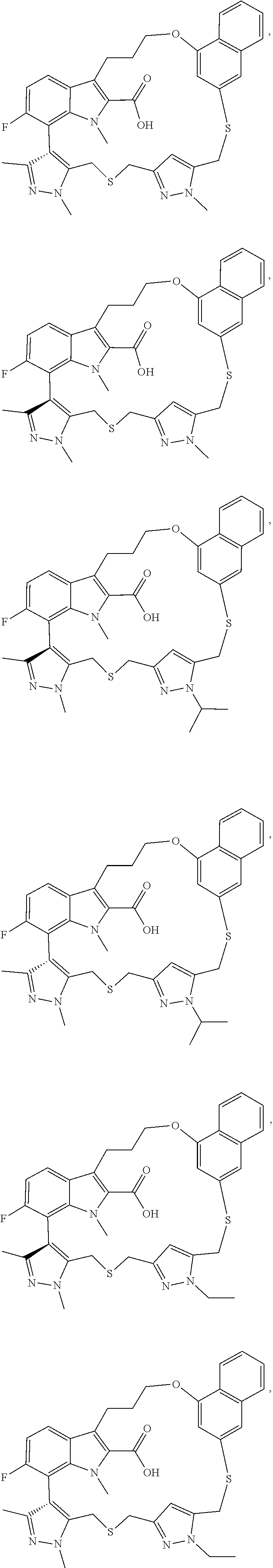 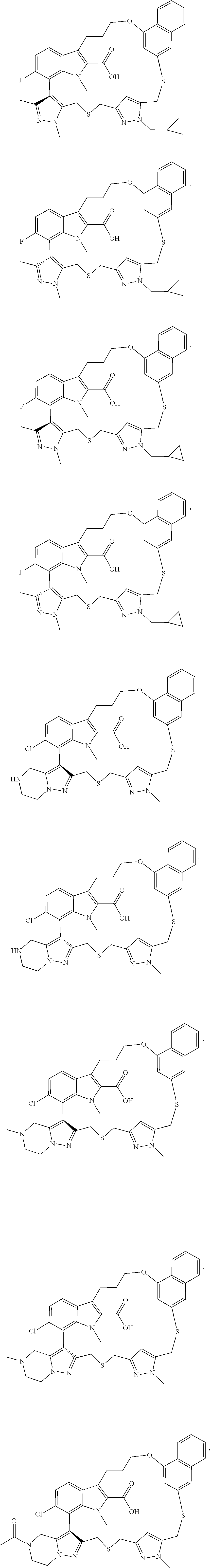 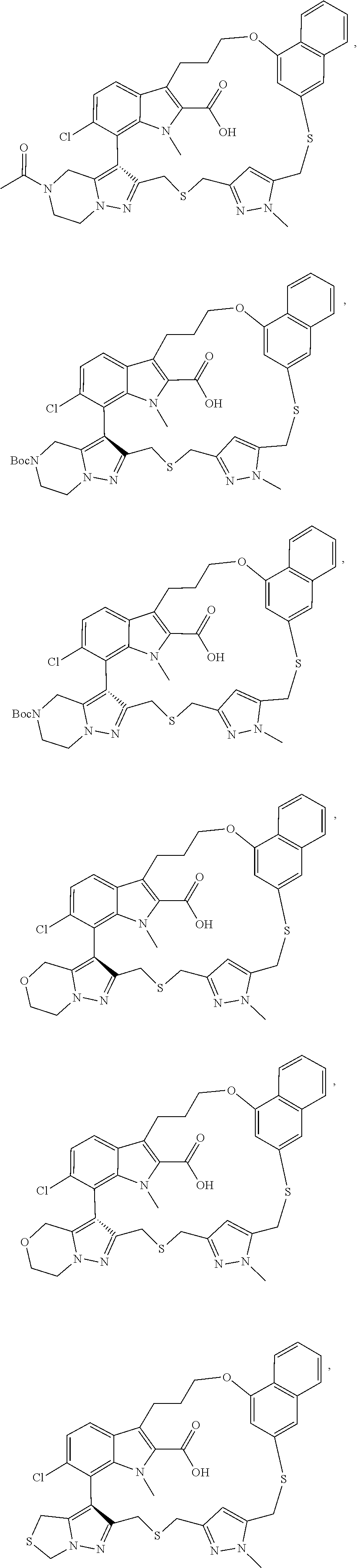 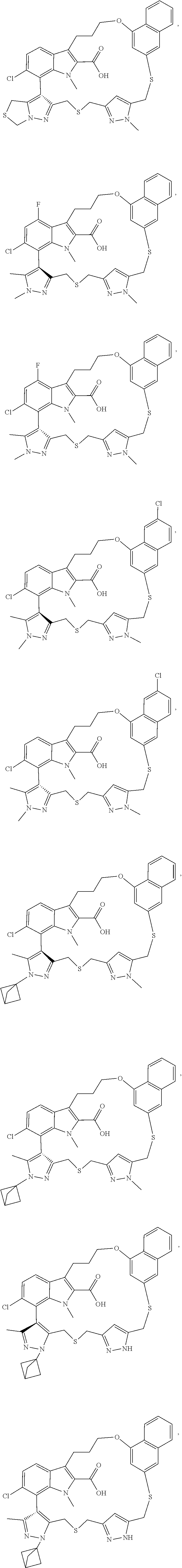 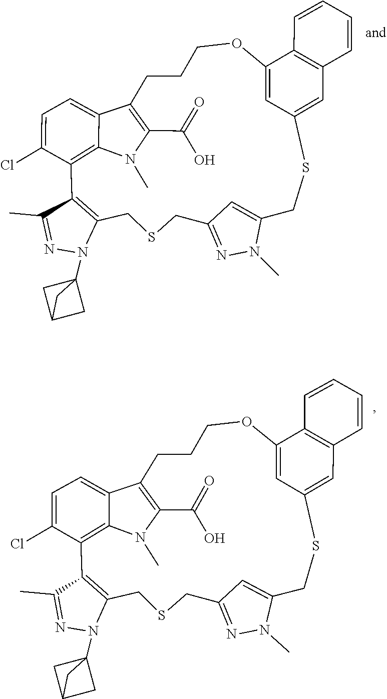
or a pharmaceutically acceptable salt of any of the foregoing.

101. A pharmaceutical composition comprising an effective amount of the compound of claim 1, or a pharmaceutically acceptable salt thereof, and a pharmaceutically acceptable carrier, diluent, excipient or combination thereof.

102. A method for ameliorating or treating a cancer comprising administering an effective amount of a compound of claim 1, or a pharmaceutically acceptable salt thereof, to a subject having the cancer, wherein the cancer is selected from the group consisting of a brain cancer, a cervicocerebral cancer, an esophageal cancer, a thyroid cancer, a small cell cancer, a non-small cell cancer, a breast cancer, a lung cancer, a stomach cancer, a gallbladder/bile duct cancer, a liver cancer, a pancreatic cancer, a colon cancer, a rectal cancer, an ovarian cancer, a choriocarcinoma, an uterus body cancer, an uterocervical cancer, a renal pelvis/ureter cancer, a bladder cancer, a prostate cancer, a penis cancer, a testicular cancer, a fetal cancer, Wilms' cancer, a skin cancer, malignant melanoma, a neuroblastoma, an osteosarcoma, an Ewing's tumor, a soft part sarcoma, an acute leukemia, a chronic lymphatic leukemia, a chronic myelocytic leukemia, polycythemia vera, a malignant lymphoma, multiple myeloma, a Hodgkin's lymphoma, and a non-Hodgkin's lymphoma.

103. A method for inhibiting replication of a malignant growth or a tumor comprising contacting the growth or the tumor with an effective amount of a compound of claim 1, or a pharmaceutically acceptable salt thereof, wherein the malignant growth or tumor is due to a cancer selected from the group consisting of a brain cancer, a cervicocerebral cancer, an esophageal cancer, a thyroid cancer, a small cell cancer, a non-small cell cancer, a breast cancer, a lung cancer, a stomach cancer, a gallbladder/bile duct cancer, a liver cancer, a pancreatic cancer, a colon cancer, a rectal cancer, an ovarian cancer, a choriocarcinoma, an uterus body cancer, an uterocervical cancer, a renal pelvis/ureter cancer, a bladder cancer, a prostate cancer, a penis cancer, a testicular cancer, a fetal cancer, Wilms' cancer, a skin cancer, malignant melanoma, a neuroblastoma, an osteosarcoma, an Ewing's tumor, a soft part sarcoma, an acute leukemia, a chronic lymphatic leukemia, a chronic myelocytic leukemia, polycythemia vera, a malignant lymphoma, multiple myeloma, a Hodgkin's lymphoma, and a non-Hodgkin's lymphoma.

104.-108. (canceled)

109. A method for inhibiting the activity of Mcl-1 comprising providing an effective amount of a compound of claim 1, or a pharmaceutically acceptable salt thereof, to a cancer cell, wherein the cancer cell is from a cancer selected from the group consisting of a brain cancer, a cervicocerebral cancer, an esophageal cancer, a thyroid cancer, a small cell cancer, a non-small cell cancer, a breast cancer, a lung cancer, a stomach cancer, a gallbladder/bile duct cancer, a liver cancer, a pancreatic cancer, a colon cancer, a rectal cancer, an ovarian cancer, a choriocarcinoma, an uterus body cancer, an uterocervical cancer, a renal pelvis/ureter cancer, a bladder cancer, a prostate cancer, a penis cancer, a testicular cancer, a fetal cancer, Wilms' cancer, a skin cancer, malignant melanoma, a neuroblastoma, an osteosarcoma, an Ewing's tumor, a soft part sarcoma, an acute leukemia, a chronic lymphatic leukemia, a chronic myelocytic leukemia, polycythemia vera, a malignant lymphoma, multiple myeloma, a Hodgkin's lymphoma, and a non-Hodgkin's lymphoma.
